(19) United States
(12) Patent Application Publication
(10) Pub. No.: US 2023/0071228 A1
(43) Pub. Date: Mar. 9, 2023

(54) 2,5-DIOXOPIPERAZINE LIPIDS WITH INTERCALATED ESTER, THIOESTER, DISULFIDE AND ANHYDRIDE MOIEITIES

(71) Applicant: Translate Bio, Inc., Lexington, MA (US)

(72) Inventors: Yi Zhang, Lexington, MA (US); Frank DeRosa, Lexington, MA (US); Shrirang Karve, Lexington, MA (US); Michael Heartlein, Lexington, MA (US); Saswata Karmakar, Lexington, MA (US); Ryan Landis, Lexington, MA (US)

(21) Appl. No.: 17/291,937

(22) PCT Filed: Nov. 7, 2019
(86) PCT No.: PCT/US19/60344

§ 371 Date: May 6, 2021

Related U.S. Application Data
(60) Provisional application No. 62/758,179, filed on Nov. 9, 2018.
(60) Provisional application No. 62/871,510, filed on Jul. 8, 2019.

(51) Int. Cl. C07D 241/04; A61K 47/24; A61K 31/7105; A61K 9/127

(52) U.S. Cl. CPC C07D 241/04; A61K 9/1272; A61K 31/7105; A61K 47/24

(57) ABSTRACT

The present invention provides, in part, cyclic amino acid lipid compounds of formula (A′), and sub-formulas thereof or a pharmaceutically acceptable salt thereof. The compounds provided herein can be useful for delivery and expression of mRNA and encoded protein, e.g., as a component of liposomal delivery vehicle, and accordingly can be useful for treating various diseases, disorders and conditions, such as those associated with deficiency of one or more proteins.

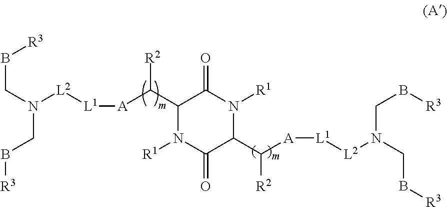

### CROSS-REFERENCE TO RELATED APPLICATIONS

The present application claims benefit of U.S. Provisional Application Nos. 62/758,179, filed Nov. 9, 2018, and 62/871,510, filed Jul. 8, 2019, each of which is incorporated by reference in its entirety.

### BACKGROUND

Delivery of nucleic acids has been explored extensively as a potential therapeutic option for certain disease states. In particular, messenger RNA (mRNA) therapy has become an increasingly important option for treatment of various diseases, including for those associated with deficiency of one or more proteins.

### SUMMARY

The present invention provides, among other things, a novel class of cyclic amino acid lipid compounds for improved in vivo delivery of therapeutic agents, such as nucleic acids. In particular, the compounds provided by the present invention are biodegradable in nature and are particularly useful for delivery of mRNA and other nucleic acids for therapeutic uses. It is contemplated that the compounds provided herein are capable of highly effective in vivo delivery while maintaining favorable toxicity profile due to the biodegradable nature.

In one aspect, the invention features a cationic lipid having a structure according to Formula (A′),

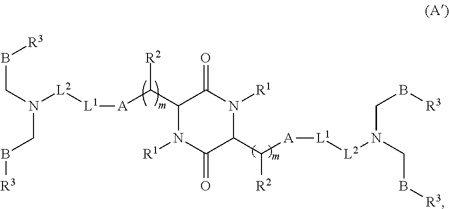

or a pharmaceutically acceptable salt thereof, wherein
each R¹ and R² is independently H or C₁-C₆ aliphatic;
each m is independently an integer having a value of 1 to 4;
each A is independently a covalent bond or arylene;
each L¹ is independently an ester, thioester, disulfide, or anhydride group;
each L² is independently C₂-C₁₀ aliphatic;
each B is independently —CHX¹— or —CH₂CO₂—;
each X¹ is independently H or OH; and
each R³ is independently C₆-C₃₀ aliphatic.

In some embodiments of Formula (A′), each R³ is independently C₆-C₂₀ aliphatic.

In embodiments, provided herein are cationic lipids having a structure according to Formula (A),

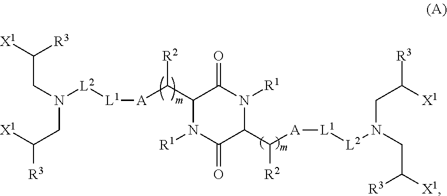

or a pharmaceutically acceptable salt thereof, wherein
each R¹ and R² is independently H or C₁-C₆ aliphatic;
each m is independently an integer having a value of 1 to 4;
each A is independently a covalent bond or arylene;
each L¹ is independently an ester, thioester, disulfide, or anhydride group;
each L² is independently C₂-C₁₀ aliphatic;
each X¹ is independently H or OH; and
each R³ is independently C₆-C₃₀ aliphatic.

In some embodiments of Formula (A), each R³ is independently C₆-C₃₀ aliphatic. In some embodiments of Formula (A), each R³ is independently C₆-C₂₀ aliphatic.

In embodiments, the cationic lipid has a structure according to Formula (A), wherein each A is independently a covalent bond or phenylene.

In embodiments, the cationic lipid has a structure according to Formula (I),

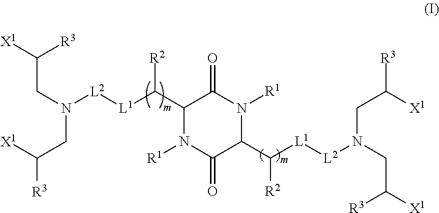

or a pharmaceutically acceptable salt thereof.

In some embodiments of Formula (I), each R³ is independently C₆-C₃₀ aliphatic. In some embodiments of Formula (I), each R³ is independently C₆-C₂₀ aliphatic.

In embodiments, the cationic lipid has a structure according to Formula (A′), (A), or (I), wherein each R¹ is H.

In embodiments, the cationic lipid has a structure according to Formula (A′), (A), or (I), wherein each R² is independently H or C₁-C₆ alkyl.

In embodiments, the cationic lipid has a structure according to Formula (A′), (A), or (I), wherein each L² is independently C₂-C₁₀ alkylene.

In embodiments, the cationic lipid has a structure according to Formula (A′), (A), or (I), wherein each R³ is independently C₆-C₂₀ alkyl, C₆-C₂₀ alkenyl, or C₆-C₂₀ alkynyl.

In embodiments, the cationic lipid has a structure according to Formula (A′), (A), or (I), wherein each X¹ is OH.

In embodiments, the cationic lipid has a structure according to Formula (A′), (A), or (I), wherein each m is 1.

In embodiments, the cationic lipid has a structure according to Formula (A′), (A), or (I), wherein each m is 2.

In embodiments, the cationic lipid has a structure according to Formula (A′), (A), or (I), wherein each m is 3.

In embodiments, the cationic lipid has a structure according to Formula (A′), (A), or (I), wherein each m is 4.

In embodiments, the cationic lipid has a structure according to Formula (I-a),

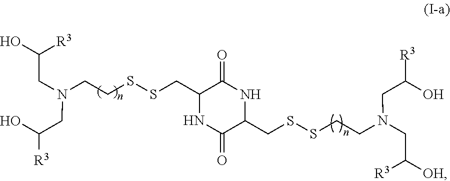

or a pharmaceutically acceptable salt thereof, wherein each n is independently an integer having a value from 1 to 9.

In some embodiments of Formula (I-a), each R³ is independently C₆-C₃₀ aliphatic. In some embodiments of Formula (I-a), each R³ is independently C₆-C₂₀ aliphatic.

In embodiments, the cationic lipid has a structure according to Formula (I-a′),

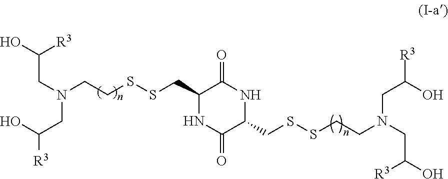

or a pharmaceutically acceptable salt thereof.

In some embodiments of Formula (I-a′), each R³ is independently C₆-C₃₀ aliphatic. In some embodiments of Formula (I-a′), each R³ is independently C₆-C₂₀ aliphatic.

In embodiments, the cationic lipid has a structure according to Formula (I-a) or (I-a′), wherein each n is 1. In embodiments, the cationic lipid has a structure according to Formula (I-a) or (I-a′), wherein each n is 2. In embodiments, the cationic lipid has a structure according to Formula (I-a) or (I-a′), wherein each n is 3.

In embodiments, the cationic lipid has a structure according to Formula (I-b),

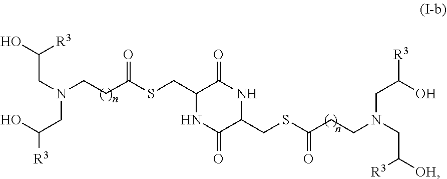

or a pharmaceutically acceptable salt thereof, wherein each n is an integer having a value of 1 to 9.

In some embodiments of Formula (I-b), each R³ is independently C₆-C₃₀ aliphatic. In some embodiments of Formula (I-b), each R³ is independently C₆-C₂₀ aliphatic.

In embodiments, the cationic lipid has a structure according to Formula (I-b′),

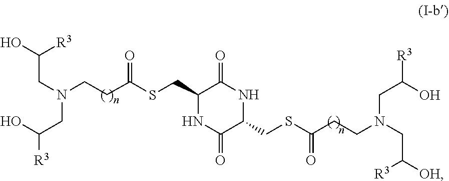

or a pharmaceutically acceptable salt thereof.

In some embodiments of Formula (I-b′), each R³ is independently C₆-C₃₀ aliphatic. In some embodiments of Formula (I-b′), each R³ is independently C₆-C₂₀ aliphatic.

In embodiments, the cationic lipid has a structure according to Formula (I-b) or (I-b′), wherein each n is 1. In embodiments, the cationic lipid has a structure according to Formula (I-b) or (I-b′), wherein each n is 2. In embodiments, the cationic lipid has a structure according to Formula (I-b) or (I-b′), wherein each n is 3.

In embodiments, the cationic lipid has a structure according to Formula (I-c),

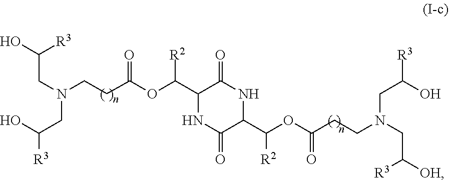

or a pharmaceutically acceptable salt thereof, wherein
each n is an integer having a value of 1 to 9; and
each R² is independently H or CH₃.

In some embodiments of Formula (I-c), each R³ is independently C₆-C₃₀ aliphatic. In some embodiments of Formula (I-c), each R³ is independently C₆-C₂₀ aliphatic.

In embodiments, the cationic lipid has a structure according to Formula (I-c′),

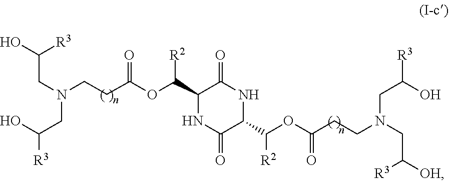

or a pharmaceutically acceptable salt thereof.

In some embodiments of Formula (I-c′), each R³ is independently C₆-C₃₀ aliphatic. In some embodiments of Formula (I-c′), each R³ is independently C₆-C₂₀ aliphatic.

In embodiments, the cationic lipid has a structure according to Formula (I-c) or (I-c′), wherein each n is 1. In embodiments, the cationic lipid has a structure according to Formula (I-c) or (I-c′), wherein each n is 2. In embodiments, the cationic lipid has a structure according to Formula (I-c) or (I-c′), wherein each n is 3.

In embodiments, the cationic lipid has a structure according to Formula (I-c) or (I-c′), wherein each R² is H.

In embodiments, the cationic lipid has a structure according to Formula (I-c-1),

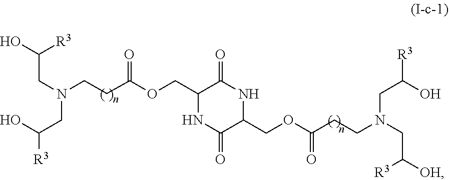

or a pharmaceutically acceptable salt thereof.

In some embodiments of Formula (I-c-1), each R³ is independently C₆-C₃₀ aliphatic. In some embodiments of Formula (I-c-1), each R³ is independently C₆-C₂₀ aliphatic.

In embodiments, the cationic lipid has a structure according to Formula (I-c′-1),

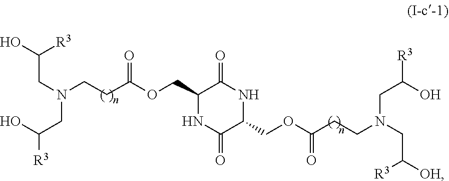

or a pharmaceutically acceptable salt thereof.

In some embodiments of Formula (I-c′-1), each R³ is independently C₆-C₃₀ aliphatic. In some embodiments of Formula (I-c′-1), each R³ is independently C₆-C₂₀ aliphatic.

In embodiments, the cationic lipid has a structure according to Formula (I-c-1) or (I-c′-1), wherein each n is 1. In embodiments, the cationic lipid has a structure according to Formula (I-c-1) or (I-c′-1), wherein each n is 2. In embodiments, the cationic lipid has a structure according to Formula (I-c-1) or (I-c′-1), wherein each n is 3

In embodiments, the cationic lipid has a structure according to Formula (I-c) or (I-c′), wherein each R² is CH₃.

In embodiments, the cationic lipid has a structure according to Formula (I-c-2),

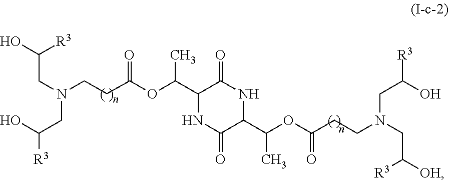

or a pharmaceutically acceptable salt thereof.

In some embodiments of Formula (I-c-2), each R³ is independently C₆-C₃₀ aliphatic. In some embodiments of Formula (I-c-2), each R³ is independently C₆-C₂₀ aliphatic.

In embodiments, the cationic lipid has a structure according to Formula (I-c′-2),

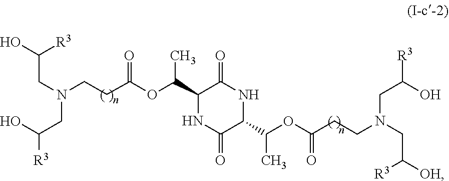

or a pharmaceutically acceptable salt thereof.

In some embodiments of Formula (I-c′-2), each R³ is independently C₆-C₃₀ aliphatic. In some embodiments of Formula (I-c′-2), each R³ is independently C₆-C₂₀ aliphatic.

In embodiments, the cationic lipid has a structure according to Formula (I-c-2) or (I-c′-2), wherein each n is 1. In embodiments, the cationic lipid has a structure according to Formula (I-c-2) or (I-c′-2), wherein each n is 2. In embodiments, the cationic lipid has a structure according to Formula (I-c-2) or (I-c′-2), wherein each n is 3

In embodiments, the cationic lipid has a structure according to Formula (I-d),

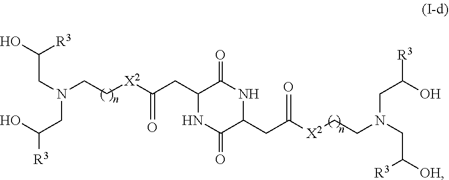

or a pharmaceutically acceptable salt thereof, wherein
each n is independently an integer having a value of 1 to 9; and
each X² is independently O or S.

In some embodiments of Formula (I-d), each R³ is independently C₆-C₃₀ aliphatic. In some embodiments of Formula (I-d), each R³ is independently C₆-C₂₀ aliphatic.

In embodiments, the cationic lipid has a structure according to Formula (I-d′),

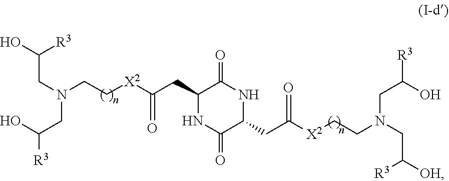

or a pharmaceutically acceptable salt thereof

In some embodiments of Formula (I-d′), each R³ is independently C₆-C₃₀ aliphatic. In some embodiments of Formula (I-d′), each R³ is independently C₆-C₂₀ aliphatic.

In embodiments, the cationic lipid has a structure according to Formula (I-d) or (I-d′), wherein each n is 1. In embodiments, the cationic lipid has a structure according to Formula (I-d) or (I-d′), wherein each n is 2. In embodiments, the cationic lipid has a structure according to Formula (I-d) or (I-d′), wherein each n is 3

In embodiments, the cationic lipid has a structure according to Formula (I-d) or (I-d′), wherein each X² is S.

In embodiments, the cationic lipid has a structure according to Formula (I-d-1),

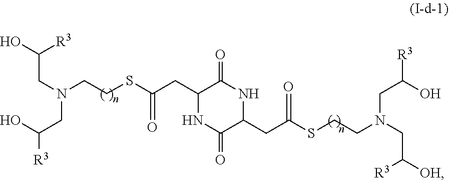

or a pharmaceutically acceptable salt thereof.

In some embodiments of Formula (I-d-1), each R³ is independently C₆-C₃₀ aliphatic. In some embodiments of Formula (I-d-1), each R³ is independently C₆-C₂₀ aliphatic.

In embodiments, the cationic lipid has a structure according to Formula (I-d) or (I-d′), wherein each X² is O.

In embodiments, the cationic lipid has a structure according to Formula (I-d-2),

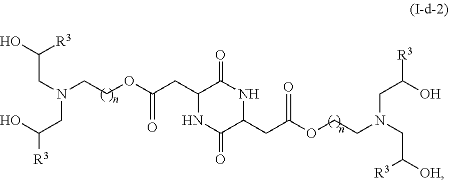

or a pharmaceutically acceptable salt thereof.

In some embodiments of Formula (I-d-2), each R³ is independently C₆-C₃₀ aliphatic. In some embodiments of Formula (I-d-2), each R³ is independently C₆-C₂₀ aliphatic.

In embodiments, the cationic lipid has a structure according to Formula (I-d) or (I-d′) (e.g., a compound of Formula (I-d-1) or (I-d-2)), wherein each n is 1.

In embodiments, the cationic lipid has a structure according to Formula (I-d) or (I-d′) (e.g., a compound of Formula (I-d-1) or (I-d-2)), wherein each n is 2.

In embodiments, the cationic lipid has a structure according to Formula (I-d) or (I-d′) (e.g., a compound of Formula (I-d-1) or (I-d-2)), wherein each n is 3.

In embodiments, the cationic lipid has a structure according to Formula (I-e),

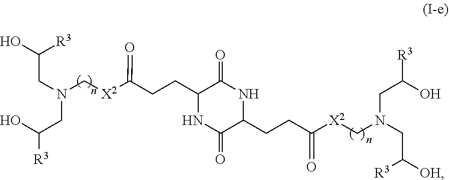

or a pharmaceutically acceptable salt thereof, wherein
each n is independently an integer of having a value of 2 to 10; and
each X² is independently 0 or S.

In some embodiments of Formula (I-e), each R³ is independently C₆-C₃₀ aliphatic. In some embodiments of Formula (I-e), each R³ is independently C₆-C₂₀ aliphatic.

In embodiments, the cationic lipid has a structure according to Formula (I-e′),

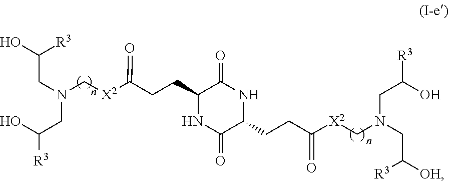

or a pharmaceutically acceptable salt thereof.

In some embodiments of Formula (I-e′), each R³ is independently C₆-C₃₀ aliphatic. In some embodiments of Formula (I-e′), each R³ is independently C₆-C₂₀ aliphatic.

In embodiments, the cationic lipid has a structure according to Formula (I-e) or (I-e′), wherein each X² is S.

In embodiments, the cationic lipid has a structure according to Formula (I-e-1),

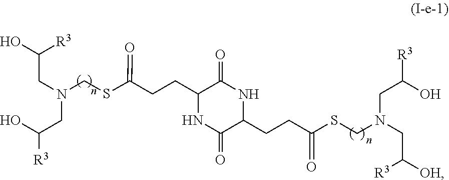

or a pharmaceutically acceptable salt thereof.

In some embodiments of Formula (I-e-1), each R³ is independently C₆-C₃₀ aliphatic. In some embodiments of Formula (I-e-1), each R³ is independently C₆-C₂₀ aliphatic.

In embodiments, the cationic lipid has a structure according to Formula (I-e) or (I-e′), wherein each X² is O.

In embodiments, the cationic lipid has a structure according to Formula (I-e-2),

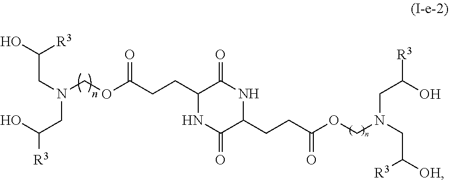

or a pharmaceutically acceptable salt thereof.

In some embodiments of Formula (I-e-2), each R³ is independently C₆-C₃₀ aliphatic. In some embodiments of Formula (I-e-2), each R³ is independently C₆-C₂₀ aliphatic.

In embodiments, the cationic lipid has a structure according to Formula (I-e) or (I-e′) (e.g., a compound of Formula (I-e-1) or (I-e-2)), wherein each n is 2.

In embodiments, the cationic lipid has a structure according to Formula (I-e) or (I-e′) (e.g., a compound of Formula (I-e-1) or (I-e-2)), wherein each n is 3.

In embodiments, the cationic lipid has a structure according to Formula (I-e) or (I-e′) (e.g., a compound of Formula (I-e-1) or (I-e-2)), wherein each n is 4.

In embodiments, the cationic lipid has a structure according to Formula (I-f),

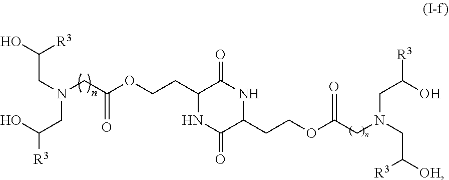

or a pharmaceutically acceptable salt thereof, wherein

each n is independently an integer of having a value of 2 to 10.

In some embodiments of Formula (I-f), each R³ is independently C₆-C₃₀ aliphatic. In some embodiments of Formula (I-f), each R³ is independently C₆-C₂₀ aliphatic.

In embodiments, the cationic lipid has a structure according to Formula (I-f′),

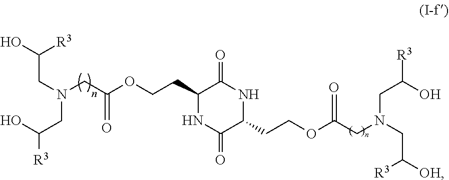

or a pharmaceutically acceptable salt thereof.

In some embodiments of Formula (I-f′), each R³ is independently C₆-C₃₀ aliphatic. In some embodiments of Formula (I-f′), each R³ is independently C₆-C₂₀ aliphatic.

In embodiments, the cationic lipid has a structure according to Formula (I-f) or (I-f′), wherein each n is 2.

In embodiments, the cationic lipid has a structure according to Formula (I-f) or (I-f′), wherein each n is 3.

In embodiments, the cationic lipid has a structure according to Formula (I-f) or (I-f′), wherein each n is 4.

In embodiments, the cationic lipid is any one of Compounds 1-552, or a pharmaceutically acceptable salt thereof.

In embodiments, the cationic lipid has a structure according to Formula (II),

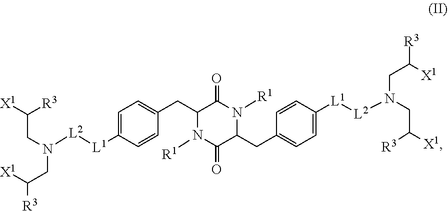

or a pharmaceutically acceptable salt thereof, wherein
each R¹ is independently H or C₁-C₆ aliphatic;
each L¹ is independently an ester, thioester, disulfide, or anhydride group;
each L² is independently C₂-C₁₀ aliphatic;
each X¹ is independently H or OH; and
each R³ is independently C₆-C₃₀ aliphatic.

In some embodiments of Formula (II), each R³ is independently C₆-C₂₀ aliphatic. In some embodiments of Formula (II), each R³ is independently C₈-C₂₀ aliphatic.

In embodiments, the cationic lipid has a structure according to Formula (II), wherein each R¹ is independently H or C₁-C₆ alkyl.

In embodiments, the cationic lipid has a structure according to Formula (II), wherein each R¹ is H.

In embodiments, the cationic lipid has a structure according to Formula (II), wherein each X¹ is OH.

In embodiments, the cationic lipid has a structure according to Formula (II-a),

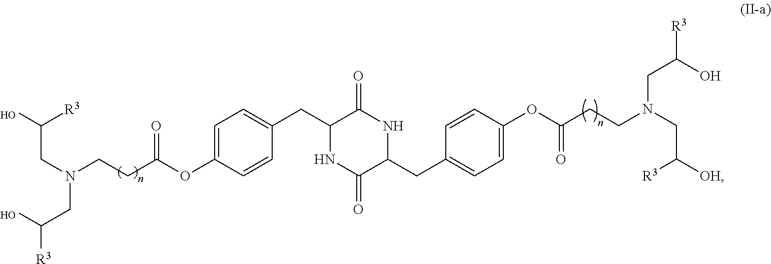

or a pharmaceutically acceptable salt thereof, wherein each n is an integer having a value of 1 to 9.

In some embodiments of Formula (II-a), each R³ is independently C₆-C₃₀ aliphatic. In some embodiments of Formula (II-a), each R³ is independently C₆-C₂₀ aliphatic. In some embodiments of Formula (II-a), each R³ is independently C₈-C₂₀ aliphatic.

In embodiments, the cationic lipid has a structure according to Formula (II-a′),

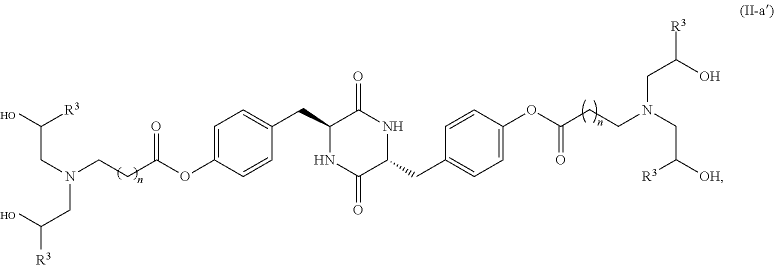

or a pharmaceutically acceptable salt thereof.

In some embodiments of Formula (II-a′), each R³ is independently C₆-C₃₀ aliphatic. In some embodiments of Formula (II-a′), each R³ is independently C₆-C₂₀ aliphatic. In some embodiments of Formula (II-a′), each R³ is independently C₈-C₂₀ aliphatic.

In embodiments, the cationic lipid has a structure according to Formula (II-a) or (II-a′), wherein each n is 1. In embodiments, the cationic lipid has a structure according to Formula (II-a) or (II-a′), wherein each n is 2. In embodiments, the cationic lipid has a structure according to Formula (II-a) or (II-a′), wherein each n is 3.

In embodiments, the cationic lipid of Formula (A′) has a structure according to Formula (III):

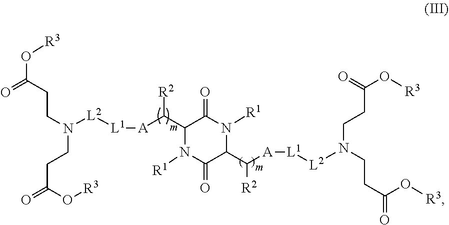

or a pharmaceutically acceptable salt thereof, wherein
each R¹ and R² is independently H or C₁-C₆ aliphatic;
each m is independently an integer having a value of 1 to 4;
each A is independently a covalent bond or arylene;
each L¹ is independently an ester, thioester, disulfide, or anhydride group;
each L² is independently C₂-C₁₀ aliphatic;
each R³ is independently C₆-C₃₀ aliphatic.

In some embodiments of Formula (III), each R³ is independently C₆-C₂₀ aliphatic.

In embodiments of Formula (III), each A is independently a covalent bond or phenylene.

In embodiments, the cationic lipid of Formula (III) has the following structure,

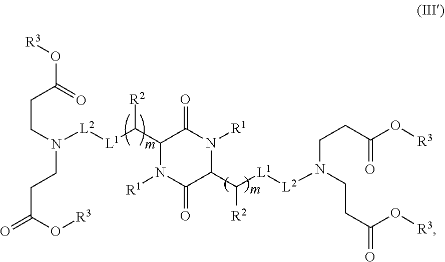

or a pharmaceutically acceptable salt thereof.

In some embodiments of Formula (III′), each R³ is independently C₆-C₃₀ aliphatic. In some embodiments of Formula (III′), each R³ is independently C₆-C₂₀ aliphatic.

In embodiments of Formula (III) or Formula (III′), each R¹ is H.

In embodiments of Formula (III) or Formula (III′), each R² is independently H or C₁-C₆ alkyl.

In embodiments of Formula (III) or Formula (III′), each L² is independently C₂-C₁₀ alkylene.

In embodiments of Formula (III) or Formula (III′), each R³ is independently C₆-C₂₀ alkyl, C₆-C₂₀ alkenyl, or C₆-C₂₀ alkynyl. In embodiments of Formula (III) or Formula (III′), R³ comprises a substituent that is —O—C(O)R′ or —C(O)—OR′, wherein R′ is C₁-C₁₆ alkyl.

In embodiments of Formula (III) or Formula (III′), each m is 1. In embodiments of Formula (III) or Formula (III′), each m is 2. In embodiments of Formula (III) or Formula (III′), each m is 3. In embodiments of Formula (III) or Formula (III′), each m is 4.

In embodiments, the cationic lipid of Formula (III) has the following structure:

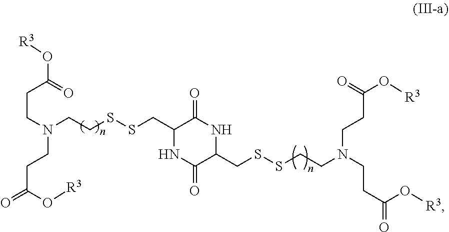

or a pharmaceutically acceptable salt thereof, wherein each n is independently an integer having a value from 1 to

In some embodiments of Formula (III-a), each R³ is independently C₆-C₃₀ aliphatic. In some embodiments of Formula (III-a), each R³ is independently C₆-C₂₀ aliphatic.

In embodiments, the cationic lipid of Formula (III) or (III-a) has the following structure:

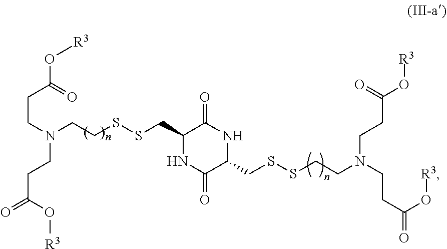

or a pharmaceutically acceptable salt thereof.

In some embodiments of Formula (III-a′), each R³ is independently C₆-C₃₀ aliphatic. In some embodiments of Formula (III-a′), each R³ is independently C₆-C₂₀ aliphatic.

In embodiments, the cationic lipid has a structure according to Formula (III-a) or (III-a′), wherein each n is 1. In embodiments, the cationic lipid has a structure according to Formula (III-a) or (III-a′), wherein each n is 2. In embodiments, the cationic lipid has a structure according to Formula (III-a) or (III-a′), wherein each n is 3.

In embodiments, the cationic lipid of Formula (III) has the following structure:

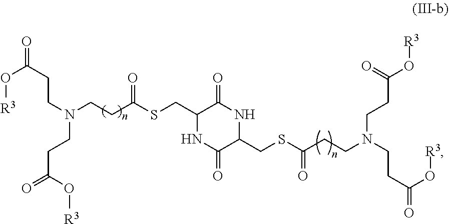

or a pharmaceutically acceptable salt thereof, wherein each n is an integer having a value of 1 to 9.

In some embodiments of Formula (III-b), each R³ is independently C₆-C₃₀ aliphatic. In some embodiments of Formula (III-b), each R³ is independently C₆-C₂₀ aliphatic.

In embodiments, the cationic lipid of Formula (III) or (III-b) has the following structure:

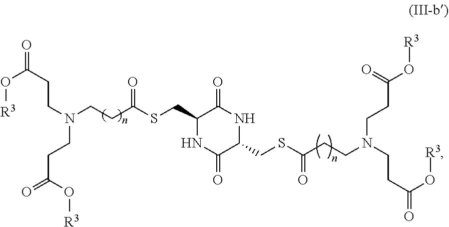

or a pharmaceutically acceptable salt thereof.

In some embodiments of Formula (III-b′), each R³ is independently C₆-C₃₀ aliphatic. In some embodiments of Formula (III-b′), each R³ is independently C₆-C₂₀ aliphatic.

In embodiments, the cationic lipid has a structure according to Formula (III-b) or (III-b′), wherein each n is 1. In embodiments, the cationic lipid has a structure according to Formula (III-b) or (III-b′), wherein each n is 2. In embodiments, the cationic lipid has a structure according to Formula (III-b) or (III-b′), wherein each n is 3.

In embodiments, the cationic lipid of Formula (III) has the following structure:

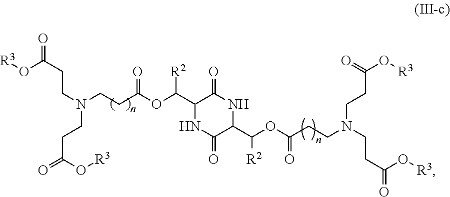

or a pharmaceutically acceptable salt thereof, wherein each n is an integer having a value of 1 to 9; and each R² is independently H or CH₃.

In some embodiments of Formula (III-c), each R³ is independently C₆-C₃₀ aliphatic. In some embodiments of Formula (III-c), each R³ is independently C₆-C₂₀ aliphatic.

In embodiments, the cationic lipid of Formula (III) or Formula (III-c) has the following structure:

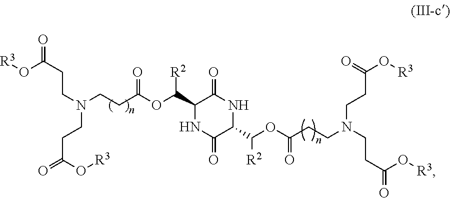

or a pharmaceutically acceptable salt thereof.

In some embodiments of Formula (III-c′), each R³ is independently C₆-C₃₀ aliphatic. In some embodiments of Formula (III-c′), each R³ is independently C₆-C₂₀ aliphatic.

In embodiments of Formula (III-c) or Formula (III-c′), each R² is H.

In embodiments, the cationic lipid of Formula (III) or Formula (III-c) has the following structure:

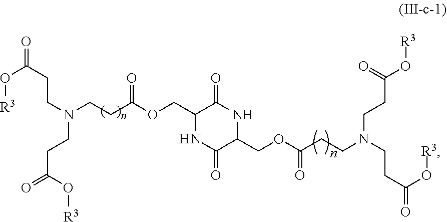

or a pharmaceutically acceptable salt thereof.

In some embodiments of Formula (III-c-1), each R³ is independently C₆-C₃₀ aliphatic. In some embodiments of Formula (III-c-1), each R³ is independently C₆-C₂₀ aliphatic.

In embodiments, the cationic lipid of Formula (III), Formula (III-c), Formula (III-c′) or Formula (III-c-1) has the following structure:

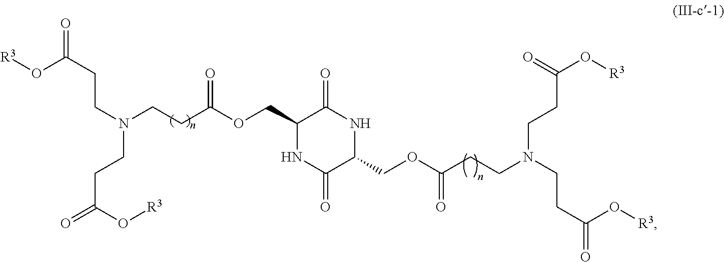

or a pharmaceutically acceptable salt thereof.

In some embodiments of Formula (III-c′-1), each R³ is independently C₆-C₃₀ aliphatic. In some embodiments of Formula (III-c′-1), each R³ is independently C₆-C₂₀ aliphatic.

In embodiments of Formula (III-c) or Formula (III-c′), each R² is CH₃.

In embodiments, the cationic lipid of Formula (III) or Formula (III-c) has the following structure:

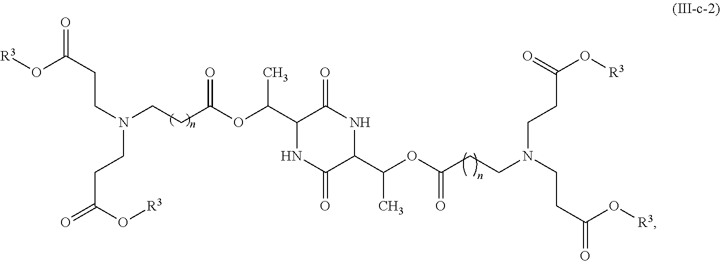

or a pharmaceutically acceptable salt thereof.

In some embodiments of Formula (III-c-2), each R³ is independently C₆-C₃₀ aliphatic. In some embodiments of Formula (III-c-2), each R³ is independently C₆-C₂₀ aliphatic.

In embodiments, the cationic lipid of Formula (III), Formula (III-c), Formula (III-c′) or Formula (III-c-2) has the following structure:

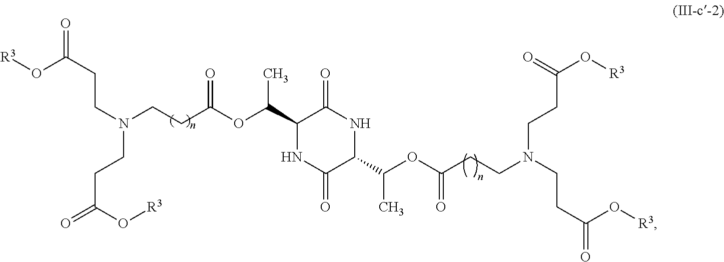

or a pharmaceutically acceptable salt thereof.

In some embodiments of Formula (III-c′-2), each R³ is independently C₆-C₃₀ aliphatic. In some embodiments of Formula (III-c′-2), each R³ is independently C₆-C₂₀ aliphatic.

In embodiments, the cationic lipid has a structure according to Formula (III-c), (III-e), (III-c-1), (III-c′-1), (III-c-2) or (III-c′-2) wherein each n is 1. In embodiments, the cationic lipid has a structure according to Formula (III-c), (III-c′), (III-c-1), (III-c′-1), (III-c-2) or (III-c′-2), wherein each n is 2. In embodiments, the cationic lipid has a structure according to Formula (III-c), (III-c′), (III-c-1), (III-c′-1), (III-c-2) or (III-c′-2), wherein each n is 3.

In embodiments, the cationic lipid of Formula (III) has the following structure:

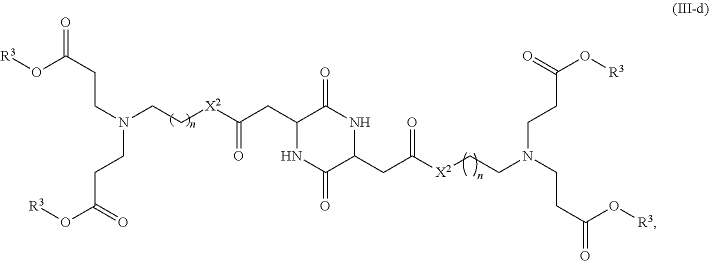

or a pharmaceutically acceptable salt thereof, wherein each n is independently an integer having a value of 1 to 9; and each X² is independently 0 or S.

In some embodiments of Formula (III-d), each R³ is independently C₆-C₃₀ aliphatic. In some embodiments of Formula (III-d), each R³ is independently C₆-C₂₀ aliphatic.

In embodiments, the cationic lipid of Formula (III) or Formula (III-d) has the following structure:

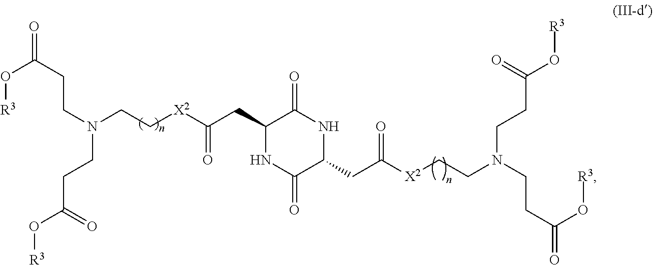

or a pharmaceutically acceptable salt thereof.

In some embodiments of Formula (III-d′), each R³ is independently C₆-C₃₀ aliphatic. In some embodiments of Formula (III-d′), each R³ is independently C₆-C₂₀ aliphatic.

In embodiments, the cationic lipid has a structure according to Formula (III-d) or (III-d′), wherein each n is 1. In embodiments, the cationic lipid has a structure according to Formula (III-d) or (III-d′), wherein each n is 2. In embodiments, the cationic lipid has a structure according to Formula (III-d) or (III-d′), wherein each n is 3.

In embodiments of Formula (III-d) or Formula (III-d′), each X² is S.

In embodiments, the cationic lipid of Formula (III) or Formula (III-d) has the following structure:

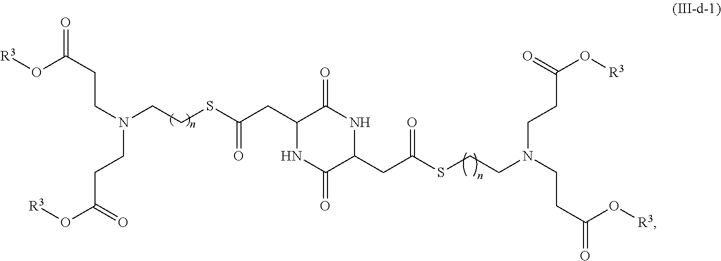

or a pharmaceutically acceptable salt thereof.

In some embodiments of Formula (III-d-1), each R³ is independently C₆-C₃₀ aliphatic. In some embodiments of Formula (III-d-1), each R³ is independently C₆-C₂₀ aliphatic.

In embodiments of Formula (III-d) or Formula (III-d′), each X² is O.

In embodiments, the cationic lipid of Formula (III) or Formula (III-d) has the following structure:

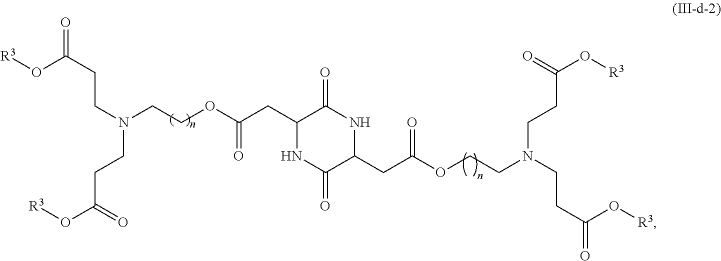

or a pharmaceutically acceptable salt thereof.

In some embodiments of Formula (III-d-2), each R³ is independently C₆-C₃₀ aliphatic. In some embodiments of Formula (III-d-2), each R³ is independently C₆-C₂₀ aliphatic.

In embodiments, the cationic lipid has a structure according to Formula (III-d-1) or (III-d-2), wherein each n is 1. In embodiments, the cationic lipid has a structure according to Formula (III-d-1) or (III-d-2), wherein each n is 2. In embodiments, the cationic lipid has a structure according to Formula (III-d-1) or (III-d-2), wherein each n is 3.

In embodiments, the cationic lipid of Formula (III) has the following structure:

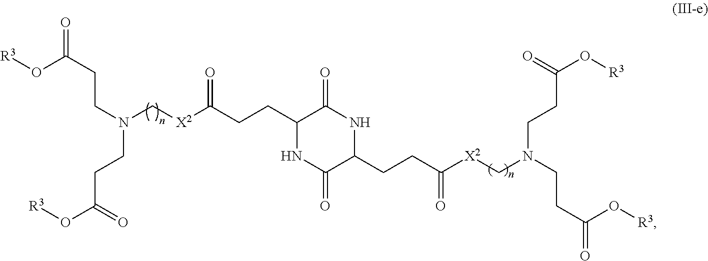

or a pharmaceutically acceptable salt thereof, wherein each n is independently an integer of having a value of 2 to 10; and each X² is independently O or S.

In some embodiments of Formula (III-e), each R³ is independently C₆-C₃₀ aliphatic. In some embodiments of Formula (III-e), each R³ is independently C₆-C₂₀ aliphatic.

In embodiments, the cationic lipid of Formula (III) or Formula (III-e) has the following structure:

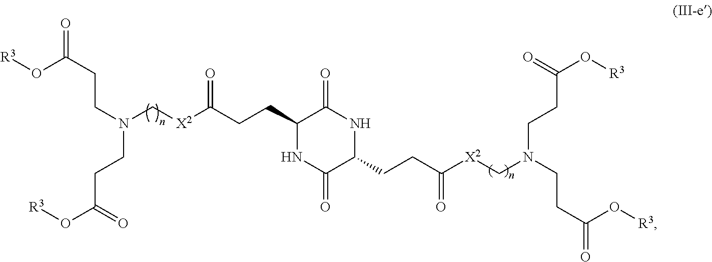

or a pharmaceutically acceptable salt thereof.

In some embodiments of Formula (III-e′), each R³ is independently C₆-C₃₀ aliphatic. In some embodiments of Formula (III-e′), each R³ is independently C₆-C₂₀ aliphatic.

In embodiments of Formula (III-e) or Formula (III-e′), each n is 2. In embodiments of Formula (III-e) or Formula (III-e′), each n is 3. In embodiments of Formula (III-e) or Formula (III-e′), each n is 4.

In embodiments of Formula (III-e) or Formula (III-e′), each X² is S.

In embodiments, the cationic lipid of Formula (III) or Formula (III-e) has the following structure:

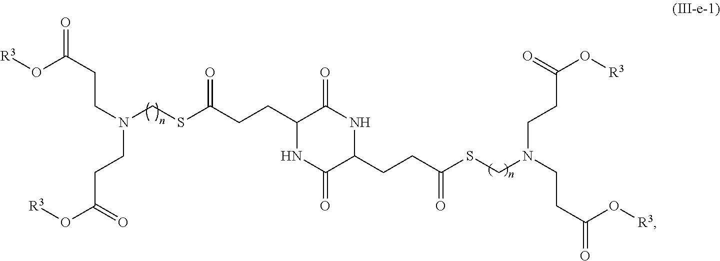

or a pharmaceutically acceptable salt thereof.

In some embodiments of Formula (III-e-1), each R³ is independently C₆-C₃₀ aliphatic. In some embodiments of Formula (III-e-1), each R³ is independently C₆-C₂₀ aliphatic.

In embodiments of Formula (III-e) or Formula (III-e′), each X² is O.

In embodiments, the cationic lipid of Formula (III) or Formula (III-e) has the following structure:

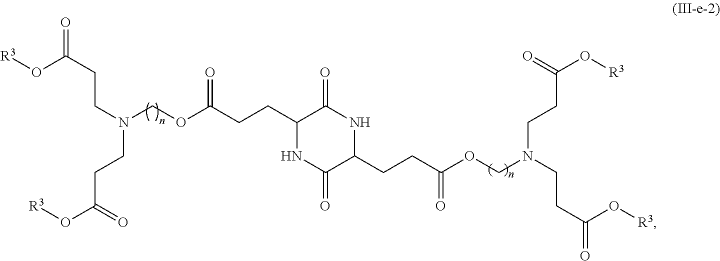

or a pharmaceutically acceptable salt thereof.

In some embodiments of Formula (III-e-2), each R³ is independently C₆-C₃₀ aliphatic. In some embodiments of Formula (III-e-2), each R³ is independently C₆-C₂₀ aliphatic.

In embodiments, the cationic lipid has a structure according to Formula (III-e-1) or (III-e-2), wherein each n is 2. In embodiments, the cationic lipid has a structure according to Formula (III-e-1) or (III-e-2), wherein each n is 3. In embodiments, the cationic lipid has a structure according to Formula (III-e-1) or (III-e-2), wherein each n is 4.

In embodiments, the cationic lipid of Formula (III) has the following structure:

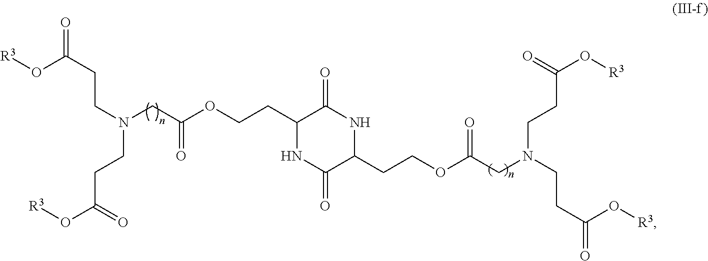

or a pharmaceutically acceptable salt thereof, wherein each n is independently an integer of having a value of 2 to 10.

In some embodiments of Formula (III-f), each R³ is independently C₆-C₃₀ aliphatic. In some embodiments of Formula (III-f), each R³ is independently C₆-C₂₀ aliphatic.

In embodiments, the cationic lipid of Formula (III) or Formula (III-f) has the following structure:

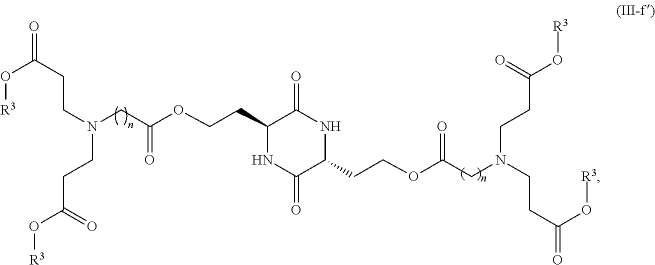

or a pharmaceutically acceptable salt thereof.

In some embodiments of Formula (III-f′), each R³ is independently C₆-C₃₀ aliphatic. In some embodiments of Formula (III-f′), each R³ is independently C₆-C₂₀ aliphatic.

In embodiments of Formula (III-f′) or Formula (III-f′), each n is 2. In embodiments of Formula (III-f) or Formula (III-f′), each n is 3. In embodiments of Formula (III-f) or Formula (III-f′), each n is 4.

In embodiments, the cationic lipid of Formula (A′) has the following structure:

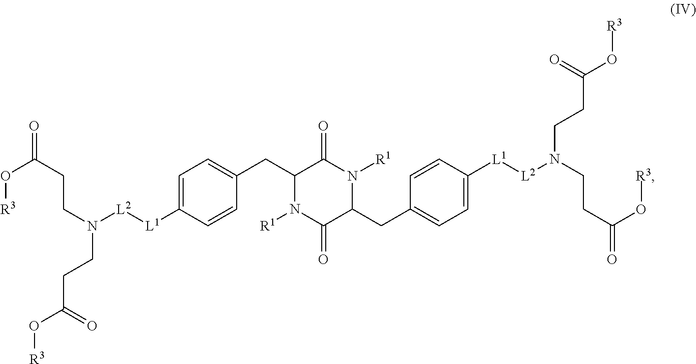

or a pharmaceutically acceptable salt thereof, wherein
each R¹ is independently H or C₁-C₆ aliphatic;
each L¹ is independently an ester, thioester, disulfide, or anhydride group;
each L² is independently C₂-C₁₀ aliphatic;
each R³ is independently C₆-C₃₀ aliphatic.

In some embodiments of Formula (IV), each R³ is independently C₆-C₂₀ aliphatic. In some embodiments of Formula (IV), each R³ is independently C₈-C₂₀ aliphatic.

In embodiments of Formula (IV), each R¹ is independently H or C₁-C₆ alkyl. In embodiments of Formula (IV), each R¹ is H.

In embodiments, the cationic lipid of Formula (IV) has the following structure:

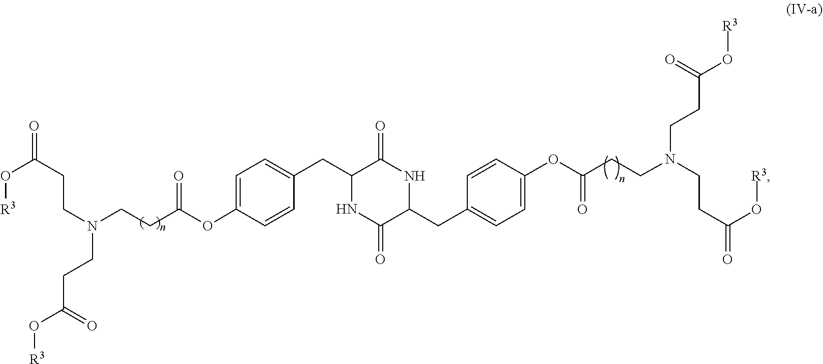

or a pharmaceutically acceptable salt thereof, wherein each n is an integer having a value of 1 to 9.

In some embodiments of Formula (IV-a), each R³ is independently C₆-C₃₀ aliphatic. In some embodiments of Formula (IV-a), each R³ is independently C₆-C₂₀ aliphatic. In some embodiments of Formula (IV-a), each R³ is independently C₈-C₂₀ aliphatic.

In embodiments, the cationic lipid of Formula (IV) or Formula (IV-a) has the following structure:

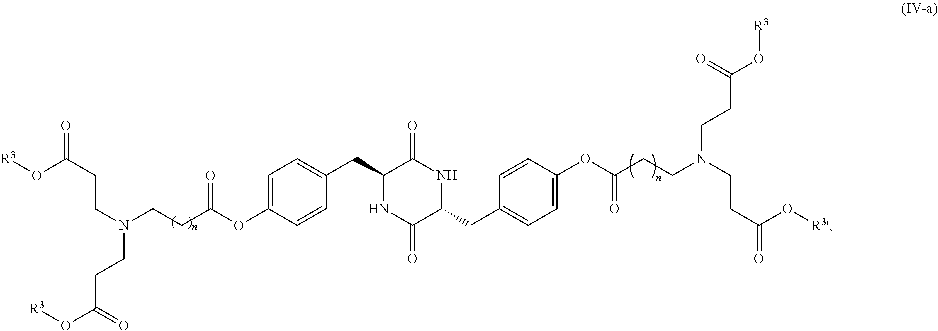

or a pharmaceutically acceptable salt thereof.

In some embodiments of Formula (IV-a′), each R³ is independently C₆-C₃₀ aliphatic. In some embodiments of Formula (IV-a′), each R³ is independently C₆-C₂₀ aliphatic. In some embodiments of Formula (IV-a′), each R³ is independently C₈-C₂₀ aliphatic.

In embodiments, the cationic lipid has a structure according to Formula (IV-a) or (IV-a′), wherein each n is 1. In embodiments, the cationic lipid has a structure according to Formula (IV-a) or (IV-a′), wherein each n is 2. In embodiments, the cationic lipid has a structure according to Formula (IV-a) or (IV-a′), wherein each n is 3.

In embodiments of any formula described herein (e.g., any of Formula (A′), (A), (I), (I-a), (I-a′), (I-b), (I-b′), (I-c), (I-c′), (I-c-1), (I-c′-1), (I-c-2), (I-c′-2), (I-d), (I-d′), (I-d-1), (I-d-2), (I-e), (I-e′), (I-e-1), (I-e-2), (I-f), (I-f′), (II), (II-a), (II-a′), (III), (III′), (III-a), (III-a′), (III-b), (III-b′), (III-c), (III-c-1), (III-c′-1), (III-c-2), (III-c′-2), (III-c′), (III-d), (III-d′), (III-d-1), (III-d-2), (III-e), (III-e′), (III-e-1), (III-e-2), (III-f), (III-f′), (IV), (IV-a), or (IV-al), each R³ is unsubstituted C₆-C₂₀ alkyl (e.g., each R³ is C₆H₁₃, C₈₁H₁₇, C₁₀H₂₁, C₁₂H₂₅, C₁₄H₂₉, C₁₆H₃₃, or C₁₈H₃₇). In embodiments, each R³ is C₁₀H₂₁.

In embodiments of any formula described herein (e.g., any of Formula (A′), (A), (I), (I-a), (I-a′), (I-b), (I-b′), (I-c), (I-c′), (I-c-1), (I-c′-1), (I-c-2), (I-c′-2), (I-d), (I-d′), (I-d-1), (I-d-2), (I-e), (I-e′), (I-e-1), (I-e-2), (I-f), (IT), (II), (II-a), (II-a′), (III), (III′), (III-a), (III-a′), (III-b), (III-b′), (III-c), (III-c-1), (III-c′-1), (III-c-2), (III-c′-2), (III-c′), (III-d), (III-d′), (III-d-1), (III-d-2), (III-e), (III-e′), (III-e-1), (III-e-2), (III-f), (III-f′), (IV), (IV-a), or (IV-al), each R³ is substituted C₆-C₂₀ alkyl. In embodiments, R³ comprises a substituent that is —O—C(O)R′ or —C(O)—OR′, wherein R′ is C₁-C₆ alkyl. In embodiments, R³ is C₆-C₁₀ alkyl substituted by —O—C(O)C₇H₁₅ or —C(O)—O—(CH₂)₂CH(C₅H₁₁)₂. In embodiments, each R³ is —(CH₂)₉—O—C(O)C₇H₁₅ or —(CH₂)₈C(O)—O—(CH₂)₂CH(C₅H₁₁)₂.

In embodiments of any formula described herein (e.g., any of Formula (A′), (A), (I), (I-a), (I-a′), (I-b), (I-b′), (I-c), (I-c′), (I-c-1), (I-c′-1), (I-c-2), (I-c′-2), (I-d), (I-d′), (I-d-1), (I-d-2), (I-e), (I-e′), (I-e-1), (I-e-2), (I-f), (I-f′), (II), (II-a), (II-a′), (III), (III′), (III-a), (III-a′), (III-b), (III-b′), (III-c), (III-c-1), (III-c′-1), (III-c-2), (III-c′-2), (III-c′), (III-d), (III-d′), (III-d-1), (III-d-2), (III-e), (III-e′), (III-e-1), (III-e-2), (III-f), (III-f′), (IV), (IV-a), or (IV-al), each R³ is unsubstituted C₆-C₂₀ alkenyl (e.g., each R³ is C₁₆-C₃₁ or C₁₆H₂₉). In embodiments, each R³ is unsubstituted monoalkenyl, unsubstituted dienyl, or unsubstituted trienyl. In embodiments, each R³ is —(CH₂)ₒR′, wherein o is 6, 7, 8, 9, or 10, and R′ is

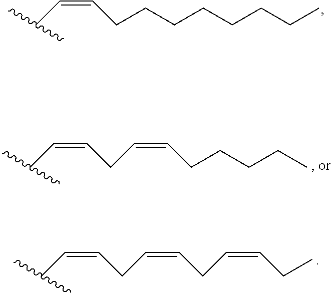 [SMILES: */C=C\CCCCCCCC ; */C=C\C/C=C\CCCCC ; */C=C\C/C=C\C/C=C\CC]

In embodiments, o is 6. In embodiments, o is 7. In embodiments, o is 8. In embodiments, o is 9. In embodiments, o is 10. In embodiments, R′ is

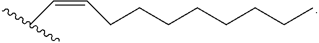 [SMILES: */C=C\CCCCCCCC]

In embodiments, R′ is

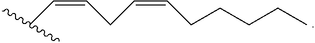 [SMILES: */C=C\C/C=C\CCCCC]

In embodiments, R′ is

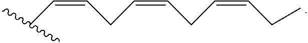 [SMILES: */C=C\C/C=C\C/C=C\CC]

In embodiments of any formula described herein (e.g., any of Formula (A′), (A), (I), (I-a), (I-a′), (I-b), (I-b′), (I-c), (I-c′), (I-c-1), (I-c′-1), (I-c-2), (I-c′-2), (I-d), (I-d′), (I-d-1), (I-d-2), (I-e), (I-e′), (I-e-1), (I-e-2), (I-f), (I-f′), (II), (II-a), (II-a′), (III), (III′), (III-a), (III-a′), (III-b), (III-b′), (III-c), (III-c-1), (III-c′-1), (III-c-2), (III-c′-2), (III-c′), (III-d), (III-d′), (III-d-1), (III-d-2), (III-e), (III-e′), (III-e-1), (III-e-2), (III-f), (III-f′), (IV), (IV-a), or (IV-a′)), each R³ is unsubstituted C₆-C₂₀ alkynyl.

In embodiments, the cationic lipid is any one of Compounds 1-552, or a pharmaceutically acceptable salt thereof.

In another aspect, the invention features a composition comprising any liposome (e.g., a liposome encapsulating an mRNA encoding a protein) described herein.

In embodiments, the mRNA encodes for cystic fibrosis transmembrane conductance regulator (CFTR) protein.

In embodiments, the mRNA encodes for ornithine transcarbamylase (OTC) protein.

In another aspect, the invention features a composition comprising a nucleic acid encapsulated within a liposome as described herein.

In embodiments, the composition further comprises one more lipids selected from the group consisting of one or more cationic lipids, one or more non-cationic lipids, and one or more PEG-modified lipids. In embodiments, the composition comprises a helper lipid that is dioleoylphosphatidylethanolamine (DOPE). In embodiments, the composition comprises a helper lipid that is 1,2-dierucoyl-sn-glycero-3-phosphoethanolamine (DEPE).

In embodiments, the nucleic acid is an mRNA encoding a peptide or protein.

In embodiments, the mRNA encodes a peptide or protein for use in the delivery to or treatment of the lung of a subject or a lung cell.

In embodiments, the mRNA encodes for cystic fibrosis transmembrane conductance regulator (CFTR) protein.

In embodiments, the mRNA encodes a peptide or protein for use in the delivery to or treatment of the liver of a subject or a liver cell.

In embodiments, the mRNA encodes for ornithine transcarbamylase (OTC) protein.

In embodiments, the mRNA encodes a peptide or protein for use in vaccine.

In embodiments, the mRNA encodes an antigen.

In some aspects, the present invention provides methods of treating a disease in a subject comprising administering to the subject a composition as described herein.

### BRIEF DESCRIPTION OF DRAWINGS

FIG. 1 relates to intravenous (IV) administration of lipid nanoparticle formulations comprising exemplary cyclic amino acid cationic lipids described herein and ornithine transcarbamylase (hOTC) mRNA. These exemplary compositions were effected for delivering mRNA in vivo and resulted in expression of hOTC in CD1 mice.

FIG. 2 relates to intratracheal aerosol administration of lipid nanoparticle formulations comprising exemplary cyclic amino acid cationic lipids described herein and firefly lucerifase (FFL) mRNA. These exemplary compositions were effected for delivering mRNA to the lung based on positive lucerifase activity.

### DETAILED DESCRIPTION OF CERTAIN EMBODIMENTS

### Definitions

In order for the present invention to be more readily understood, certain terms are first defined below. Additional definitions for the following terms and other terms are set forth throughout the specification. The publications and other reference materials referenced herein to describe the background of the invention and to provide additional detail regarding its practice are hereby incorporated by reference.

Amino acid: As used herein, the term “amino acid,” in its broadest sense, refers to any compound and/or substance that can be incorporated into a polypeptide chain. In some embodiments, an amino acid has the general structure H₂N—C(H)(R)—COOH. In some embodiments, an amino acid is a naturally occurring amino acid. In some embodiments, an amino acid is a nonstandard amino acid. In some embodiments, an amino acid is a synthetic amino acid; in some embodiments, an amino acid is a d-amino acid; in some embodiments, an amino acid is an I-amino acid. “Standard amino acid” refers to any of the twenty standard I-amino acids commonly found in naturally occurring peptides. “Nonstandard amino acid” refers to any amino acid, other than the standard amino acids, regardless of whether it is prepared synthetically or obtained from a natural source. As used herein, “synthetic amino acid” encompasses chemically modified amino acids, including but not limited to salts, amino acid derivatives (such as amides), and/or substitutions. Amino acids, including carboxy- and/or amino-terminal amino acids in peptides, can be modified by methylation, amidation, acetylation, protecting groups, and/or substitution with other chemical groups that can change the peptide's circulating half-life without adversely affecting their activity. Amino acids may participate in a disulfide bond. Amino acids may comprise one or posttranslational modifications, such as association with one or more chemical entities (e.g., methyl groups, acetate groups, acetyl groups, phosphate groups, formyl moieties, isoprenoid groups, sulfate groups, polyethylene glycol moieties, lipid moieties, carbohydrate moieties, biotin moieties, etc.). The term “amino acid” is used interchangeably with “amino acid residue,” and may refer to a free amino acid and/or to an amino acid residue of a peptide. It will be apparent from the context in which the term is used whether it refers to a free amino acid or a residue of a peptide.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically-engineered animal, and/or a clone.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Biologically active: As used herein, the term “biologically active” refers to a characteristic of any agent that has activity in a biological system, and particularly in an organism. For instance, an agent that, when administered to an organism, has a biological effect on that organism, is considered to be biologically active.

Delivery: As used herein, the term “delivery” encompasses both local and systemic delivery. For example, delivery of mRNA encompasses situations in which an mRNA is delivered to a target tissue and the encoded protein is expressed and retained within the target tissue (also referred to as “local distribution” or “local delivery”), and situations in which an mRNA is delivered to a target tissue and the encoded protein is expressed and secreted into patient's circulation system (e.g., serum) and systematically distributed and taken up by other tissues (also referred to as “systemic distribution” or “systemic delivery”).

Expression: As used herein, “expression” of a nucleic acid sequence refers to translation of an mRNA into a polypeptide, assemble multiple polypeptides into an intact protein (e.g., enzyme) and/or post-translational modification of a polypeptide or fully assembled protein (e.g., enzyme). In this application, the terms “expression” and “production,” and grammatical equivalent, are used inter-changeably.

Functional: As used herein, a “functional” biological molecule is a biological molecule in a form in which it exhibits a property and/or activity by which it is characterized.

Half-life: As used herein, the term “half-life” is the time required for a quantity such as nucleic acid or protein concentration or activity to fall to half of its value as measured at the beginning of a time period.

Helper lipid: The term “helper lipid” as used herein refers to any neutral or zwitterionic lipid material including cholesterol. Without wishing to be held to a particular theory, helper lipids may add stability, rigidity, and/or fluidity within lipid bilayers/nanoparticles.

Improve, increase, or reduce: As used herein, the terms “improve,” “increase” or “reduce,” or grammatical equivalents, indicate values that are relative to a baseline measurement, such as a measurement in the same individual prior to initiation of the treatment described herein, or a measurement in a control subject (or multiple control subject) in the absence of the treatment described herein. A “control subject” is a subject afflicted with the same form of disease as the subject being treated, who is about the same age as the subject being treated.

In Vitro: As used herein, the term “in vitro” refers to events that occur in an artificial environment, e.g., in a test tube or reaction vessel, in cell culture, etc., rather than within a multi-cellular organism.

In Vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

Isolated: As used herein, the term “isolated” refers to a substance and/or entity that has been (1) separated from at least some of the components with which it was associated when initially produced (whether in nature and/or in an experimental setting), and/or (2) produced, prepared, and/or manufactured by the hand of man. isolated substances and/or entities may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of the other components with which they were initially associated. In some embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. As used herein, calculation of percent purity of isolated substances and/or entities should not include excipients (e.g., buffer, solvent, water, etc.).

Liposome: As used herein, the term “liposome” refers to any lamellar, multilamellar, or solid nanoparticle vesicle. Typically, a liposome as used herein can be formed by mixing one or more lipids or by mixing one or more lipids and polymer(s). In some embodiments, a liposome suitable for the present invention contains a cationic lipids(s) and optionally non-cationic lipid(s), optionally cholesterol-based lipid(s), and/or optionally PEG-modified lipid(s).

messenger RNA (mRNA): As used herein, the term “messenger RNA (mRNA)” or “mRNA” refers to a polynucleotide that encodes at least one polypeptide. mRNA as used herein encompasses both modified and unmodified RNA. The term “modified mRNA” related to mRNA comprising at least one chemically modified nucleotide. mRNA may contain one or more coding and non-coding regions. mRNA can be purified from natural sources, produced using recombinant expression systems and optionally purified, chemically synthesized, etc. Where appropriate, e.g., in the case of chemically synthesized molecules, mRNA can comprise nucleoside analogs such as analogs having chemically modified bases or sugars, backbone modifications, etc. An mRNA sequence is presented in the 5′ to 3′ direction unless otherwise indicated. In some embodiments, an mRNA is or comprises natural nucleosides (e.g., adenosine, guanosine, cytidine, uridine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

Nucleic acid: As used herein, the term “nucleic acid,” in its broadest sense, refers to any compound and/or substance that is or can be incorporated into a polynucleotide chain. In some embodiments, a nucleic acid is a compound and/or substance that is or can be incorporated into a polynucleotide chain via a phosphodiester linkage. In some embodiments, “nucleic acid” refers to individual nucleic acid residues (e.g., nucleotides and/or nucleosides). In some embodiments, “nucleic acid” refers to a polynucleotide chain comprising individual nucleic acid residues. In some embodiments, “nucleic acid” encompasses RNA as well as single and/or double-stranded DNA and/or cDNA. In some embodiments, “nucleic acid” encompasses ribonucleic acids (RNA), including but not limited to any one or more of interference RNAs (RNAi), small interfering RNA (siRNA), short hairpin RNA (shRNA), antisense RNA (aRNA), messenger RNA (mRNA), modified messenger RNA (mmRNA), long non-coding RNA (IncRNA), micro-RNA (miRNA) multimeric coding nucleic acid (MCNA), polymeric coding nucleic acid (PCNA), guide RNA (gRNA) and CRISPR RNA (crRNA). In some embodiments, “nucleic acid” encompasses deoxyribonucleic acid (DNA), including but not limited to any one or more of single-stranded DNA (ssDNA), double-stranded DNA (dsDNA) and complementary DNA (cDNA). In some embodiments, “nucleic acid” encompasses both RNA and DNA. In embodiments, DNA may be in the form of antisense DNA, plasmid DNA, parts of a plasmid DNA, pre-condensed DNA, a product of a polymerase chain reaction (PCR), vectors (e.g., P1, PAC, BAC, YAC, artificial chromosomes), expression cassettes, chimeric sequences, chromosomal DNA, or derivatives of these groups. In embodiments, RNA may be in the form of messenger RNA (mRNA), ribosomal RNA (rRNA), signal recognition particle RNA (7 SL RNA or SRP RNA), transfer RNA (tRNA), transfer-messenger RNA (tmRNA), small nuclear RNA (snRNA), small nucleolar RNA (snoRNA), SmY RNA, small Cajal body-specific RNA (scaRNA), guide RNA (gRNA), ribonuclease P (RNase P), Y RNA, telomerase RNA component (TERC), spliced leader RNA (SL RNA), antisense RNA (aRNA or asRNA), cis-natural antisense transcript (cis-NAT), CRISPR RNA (crRNA), long noncoding RNA (lncRNA), micro-RNA (miRNA), piwi-interacting RNA (piRNA), small interfering RNA (siRNA), transacting siRNA (tasiRNA), repeat associated siRNA (rasiRNA), 73K RNA, retrotransposons, a viral genome, a viroid, satellite RNA, or derivatives of these groups. In some embodiments, a nucleic acid is a mRNA encoding a protein such as an enzyme.

Patient: As used herein, the term “patient” or “subject” refers to any organism to which a provided composition may be administered, e.g., for experimental, diagnostic, prophylactic, cosmetic, and/or therapeutic purposes. Typical patients include animals (e.g., mammals such as mice, rats, rabbits, non-human primates, and/or humans). In some embodiments, a patient is a human. A human includes pre- and post-natal forms.

Pharmaceutically acceptable: The term “pharmaceutically acceptable” as used herein, refers to substances that, within the scope of sound medical judgment, are suitable for use in contact with the tissues of human beings and animals without excessive toxicity, irritation, allergic response, or other problem or complication, commensurate with a reasonable benefit/risk ratio.

Pharmaceutically acceptable salt: Pharmaceutically acceptable salts are well known in the art. For example, S. M. Berge et al. describes pharmaceutically acceptable salts in detail in J. Pharmaceutical Sciences (1977) 66:1-19. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or rnalonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, sulfonate and aryl sulfonate. Further pharmaceutically acceptable salts include salts formed from the quarternization of an amine using an appropriate electrophile, e.g., an alkyl halide, to form a quarternized alkylated amino salt.

Systemic distribution or delivery: As used herein, the terms “systemic distribution,” “systemic delivery,” or grammatical equivalent, refer to a delivery or distribution mechanism or approach that affect the entire body or an entire organism. Typically, systemic distribution or delivery is accomplished via body's circulation system, e.g., blood stream. Compared to the definition of “local distribution or delivery.”

Subject: As used herein, the term “subject” refers to a human or any non-human animal (e.g., mouse, rat, rabbit, dog, cat, cattle, swine, sheep, horse or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Target tissues: As used herein, the term “target tissues” refers to any tissue that is affected by a disease to be treated. In some embodiments, target tissues include those tissues that display disease-associated pathology, symptom, or feature.

Therapeutically effective amount: As used herein, the term “therapeutically effective amount” of a therapeutic agent means an amount that is sufficient, when administered to a subject suffering from or susceptible to a disease, disorder, and/or condition, to treat, diagnose, prevent, and/or delay the onset of the symptom(s) of the disease, disorder, and/or condition. It will be appreciated by those of ordinary skill in the art that a therapeutically effective amount is typically administered via a dosing regimen comprising at least one unit dose.

Treating: As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

Aliphatic: As used herein, the term aliphatic refers to C₁-C₄₀ hydrocarbons and includes both saturated and unsaturated hydrocarbons. An aliphatic may be linear, branched, or cyclic. For example, C₁-C₂₀ aliphatics can include C₁-C₂₀ alkyls (e.g., linear or branched C₁-C₂₀ saturated alkyls), C₂-C₂₀ alkenyls (e.g., linear or branched C₄-C₂₀ dienyls, linear or branched C₆-C₂₀ trienyls, and the like), and C₂-C₂₀ alkynyls (e.g., linear or branched C₂-C₂₀ alkynyls). C₁-C₂₀ aliphatics can include C₃-C₂₀ cyclic aliphatics (e.g., C₃-C₂₀ cycloalkyls, C₄-C₂₀ cycloalkenyls, or C₈-C₂₀ cycloalkynyls). In certain embodiments, the aliphatic may comprise one or more cyclic aliphatic and/or one or more heteroatoms such as oxygen, nitrogen, or sulfur and may optionally be substituted with one or more substituents such as alkyl, halo, alkoxyl, hydroxy, amino, aryl, ether, ester or amide. An aliphatic group is unsubstituted or substituted with one or more substituent groups as described herein. For example, an aliphatic may be substituted with one or more (e.g., 1, 2, 3, 4, 5, or 6 independently selected substituents) of halogen, —COR′, —CO₂H, —CO₂R′, —CN, —OH, —OR′, —OCOR′, —OCO₂R′, —NH₂, —NHR′, —N(R′)₂, —SR′ or —SO₂R′, wherein each instance of R′ independently is C₁-C₂₀ aliphatic (e.g., C₁-C₂₀ alkyl, C₁-C₁₅ alkyl, C₁-C₁₀ alkyl, or C₁-C₃ alkyl). In embodiments, R′ independently is an unsubstituted alkyl (e.g., unsubstituted C₁-C₂₀ alkyl, C₁-C₁₅ alkyl, C₁-C₁₀ alkyl, or C₁-C₃ alkyl). In embodiments, R′ independently is unsubstituted C₁-C₃ alkyl. In embodiments, the aliphatic is unsubstituted. In embodiments, the aliphatic does not include any heteroatoms.

Alkyl: As used herein, the term “alkyl” means acyclic linear and branched hydrocarbon groups, e.g. “C₁-C₂₀ alkyl” refers to alkyl groups having 1-20 carbons. An alkyl group may be linear or branched. Examples of alkyl groups include, but are not limited to, methyl, ethyl, n-propyl, isopropyl, butyl, isobutyl, sec-butyl, tert-butyl, pentyl, isopentyl tert-pentylhexyl, Isohexyl etc. Other alkyl groups will be readily apparent to those of skill in the art given the benefit of the present disclosure. An alkyl group may be unsubstituted or substituted with one or more substituent groups as described herein. For example, an alkyl group may be substituted with one or more (e.g., 1, 2, 3, 4, 5, or 6 independently selected substituents) of halogen, —COR′, —CO₂H, —CO₂R′, —CN, —OH, —OR′, —OCOR′, —OCO₂R′, —NH₂, —NHR′, —N(R′)₂, —SR′ or —SO₂R′, wherein each instance of R′ independently is C₁-C₂₀ aliphatic (e.g., C₁-C₂₀ alkyl, C₁-C₁₅ alkyl, C₁-C₁₀ alkyl, or C₁-C₃ alkyl). In embodiments, R′ independently is an unsubstituted alkyl (e.g., unsubstituted C₁-C₂₀ alkyl, C₁-C₁₅ alkyl, C₁-C₁₀ alkyl, or C₁-C₃ alkyl). In embodiments, R′ independently is unsubstituted C₁-C₃ alkyl. In embodiments, the alkyl is substituted (e.g., with 1, 2, 3, 4, 5, or 6 substituent groups as described herein). In embodiments, an alkyl group is substituted with a —OH group and may also be referred to herein as a “hydroxyalkyl” group, where the prefix denotes the —OH group and “alkyl” is as described herein.

Affixing the suffix “-ene” to a group indicates the group is a divalent moiety, e.g., arylene is the divalent moiety of aryl, and heteroarylene is the divalent moiety of heteroaryl.

Alkylene: The term “alkylene,” as used herein, represents a saturated divalent straight or branched chain hydrocarbon group and is exemplified by methylene, ethylene, isopropylene and the like. Likewise, the term “alkenylene” as used herein represents an unsaturated divalent straight or branched chain hydrocarbon group having one or more unsaturated carbon-carbon double bonds that may occur in any stable point along the chain, and the term “alkynylene” herein represents an unsaturated divalent straight or branched chain hydrocarbon group having one or more unsaturated carbon-carbon triple bonds that may occur in any stable point along the chain. In certain embodiments, an alkylene, alkenylene, or alkynylene group may comprise one or more cyclic aliphatic and/or one or more heteroatoms such as oxygen, nitrogen, or sulfur and may optionally be substituted with one or more substituents such as alkyl, halo, alkoxyl, hydroxy, amino, aryl, ether, ester or amide. For example, an alkylene, alkenylene, or alkynylene may be substituted with one or more (e.g., 1, 2, 3, 4, 5, or 6 independently selected substituents) of halogen, —COR′, —CO₂H, —CO₂R′, —CN, —OH, —OR′, —OCOR′, —OCO₂R′, —NH₂, —NHR′, —N(R′)₂, —SR′ or —SO₂R′, wherein each instance of R′ independently is C₁-C₂₀ aliphatic (e.g., C₁-C₂₀ alkyl, C₁-C₁₅ alkyl, C₁-C₁₀ alkyl, or C₁-C₃ alkyl). In embodiments, R′ independently is an unsubstituted alkyl (e.g., unsubstituted C₁-C₂₀ alkyl, C₁-C₁₅ alkyl, C₁-C₁₀ alkyl, or C₁-C₃ alkyl). In embodiments, R′ independently is unsubstituted C₁-C₃ alkyl. In certain embodiments, an alkylene, alkenylene, or alkynylene is unsubstituted. In certain embodiments, an alkylene, alkenylene, or alkynylene does not include any heteroatoms.

Alkenyl: As used herein, “alkenyl” means any linear or branched hydrocarbon chains having one or more unsaturated carbon-carbon double bonds that may occur in any stable point along the chain, e.g. “C₂-C₂₀ alkenyl” refers to an alkenyl group having 2-20 carbons. For example, an alkenyl group includes prop-2-enyl, but-2-enyl, but-3-enyl, 2-methylprop-2-enyl, hex-2-enyl, hex-5-enyl, 2,3-dimethylbut-2-enyl, and the like. In embodiments, the alkenyl comprises 1, 2, or 3 carbon-carbon double bond. In embodiments, the alkenyl comprises a single carbon-carbon double bond. In embodiments, multiple double bonds (e.g., 2 or 3) are conjugated. An alkenyl group may be unsubstituted or substituted with one or more substituent groups as described herein. For example, an alkenyl group may be substituted with one or more (e.g., 1, 2, 3, 4, 5, or 6 independently selected substituents) of halogen, —COR′, —CO₂H, —CO₂R′, —CN, —OH, —OR′, —OCOR′, —OCO₂R′, —NH₂, —NHR′, —N(R′)₂, —SR′ or —SO₂R′, wherein each instance of R′ independently is C₁-C₂₀ aliphatic (e.g., C₁-C₂₀ alkyl, C₁-C₁₅ alkyl, C₁-C₁₀ alkyl, or C₁-C₃ alkyl). In embodiments, R′ independently is an unsubstituted alkyl (e.g., unsubstituted C₁-C₂₀ alkyl, C₁-C₁₅ alkyl, C₁-C₁₀ alkyl, or C₁-C₃ alkyl). In embodiments, R′ independently is unsubstituted C₁-C₃ alkyl. In embodiments, the alkenyl is unsubstituted. In embodiments, the alkenyl is substituted (e.g., with 1, 2, 3, 4, 5, or 6 substituent groups as described herein). In embodiments, an alkenyl group is substituted with a —OH group and may also be referred to herein as a “hydroxyalkenyl” group, where the prefix denotes the —OH group and “alkenyl” is as described herein.

Alkynyl: As used herein, “alkynyl” means any hydrocarbon chain of either linear or branched configuration, having one or more carbon-carbon triple bonds occurring in any stable point along the chain, e.g. “C₂-C₂₀ alkynyl” refers to an alkynyl group having 2-20 carbons. Examples of an alkynyl group include prop-2-ynyl, but-2-ynyl, but-3-ynyl, pent-2-ynyl, 3-methylpent-4-ynyl, hex-2-ynyl, hex-5-ynyl, etc. In embodiments, an alkynyl comprises one carbon-carbon triple bond. An alkynyl group may be unsubstituted or substituted with one or more substituent groups as described herein. For example, an alkynyl group may be substituted with one or more (e.g., 1, 2, 3, 4, 5, or 6 independently selected substituents) of halogen, —COR′, —CO₂H, —CO₂R′, —CN, —OH, —OR′, —OCOR′, —OCO₂R′, —NH₂, —NHR′, —N(R′)₂, —SR′ or —SO₂R′, wherein each instance of R′ independently is C₁-C₂₀ aliphatic (e.g., C₁-C₂₀ alkyl, C₁-C₁₅ alkyl, C₁-C₁₀ alkyl, or C₁-C₃ alkyl). In embodiments, R′ independently is an unsubstituted alkyl (e.g., unsubstituted C₁-C₂₀ alkyl, C₁-C₁₅ alkyl, C₁-C₁₀ alkyl, or C₁-C₃ alkyl). In embodiments, R′ independently is unsubstituted C₁-C₃ alkyl. In embodiments, the alkynyl is unsubstituted. In embodiments, the alkynyl is substituted (e.g., with 1, 2, 3, 4, 5, or 6 substituent groups as described herein).

Aryl: The term “aryl” used alone or as part of a larger moiety as in “aralkyl,” refers to a monocyclic, bicyclic, or tricyclic carbocyclic ring system having a total of six to fourteen ring members, wherein said ring system has a single point of attachment to the rest of the molecule, at least one ring in the system is aromatic and wherein each ring in the system contains 4 to 7 ring members. In embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Exemplary aryls include phenyl, naphthyl, and anthracene.

Arylene: The term “arylene” as used herein refers to an aryl group that is divalent (that is, having two points of attachment to the molecule). Exemplary arylenes include phenylene (e.g., unsubstituted phenylene or substituted phenylene).

Halogen: As used herein, the term “halogen” means fluorine, chlorine, bromine, or iodine.

Heteroalkyl: The term “heteroalkyl” is meant a branched or unbranched alkyl, alkenyl, or alkynyl group having from 1 to 14 carbon atoms in addition to 1, 2, 3 or 4 heteroatoms independently selected from the group consisting of N, O, S, and P. Heteroalkyls include tertiary amines, secondary amines, ethers, thioethers, amides, thioamides, carbamates, thiocarbamates, hydrazones, imines, phosphodiesters, phosphoramidates, sulfonamides, and disulfides. A heteroalkyl group may optionally include monocyclic, bicyclic, or tricyclic rings, in which each ring desirably has three to six members. Examples of heteroalkyls include polyethers, such as methoxymethyl and ethoxyethyl.

Heteroalkylene: The term “heteroalkylene,” as used herein, represents a divalent form of a heteroalkyl group as described herein.

### Compounds of the Invention

Liposomal-based vehicles are considered an attractive carrier for therapeutic agents and remain subject to continued development efforts. While liposomal-based vehicles that comprise certain lipid components have shown promising results with regards to encapsulation, stability and site localization, there remains a great need for improvement of liposomal-based delivery systems. For example, a significant drawback of liposomal delivery systems relates to the construction of liposomes that have sufficient cell culture or in vivo stability to reach desired target cells and/or intracellular compartments, and the ability of such liposomal delivery systems to efficiently release their encapsulated materials to such target cells.

In particular, there remains a need for improved lipids compounds that demonstrate improved pharmacokinetic properties and which are capable of delivering macromolecules, such as nucleic acids to a wide variety cell types and tissues with enhanced efficiency. Importantly, there also remains a particular need for novel lipid compounds that are characterized as having reduced toxicity and are capable of efficiently delivering encapsulated nucleic acids and polynucleotides to targeted cells, tissues and organs.

Described herein a novel class of cyclic amino acid lipid compounds for improved in vivo delivery of therapeutic agents, such as nucleic acids. In particular, a biodegradable compound described herein may be used to as a cationic lipid, together with other non-cationic lipids, to formulate a lipid-based nanoparticle (e.g., liposome) for encapsulating therapeutic agents, such as nucleic acids (e.g., DNA, siRNA, mRNA, microRNA) for therapeutic use.

In embodiments, compounds described herein can provide one or more desired characteristics or properties. That is, in certain embodiments, compounds described herein can be characterized as having one or more properties that afford such compounds advantages relative to other similarly classified lipids. For example, compounds disclosed herein can allow for the control and tailoring of the properties of liposomal compositions (e.g., lipid nanoparticles) of which they are a component. In particular, compounds disclosed herein can be characterized by enhanced transfection efficiencies and their ability to provoke specific biological outcomes. Such outcomes can include, for example enhanced cellular uptake, endosomal/lysosomal disruption capabilities and/or promoting the release of encapsulated materials (e.g., polynucleotides) intracellularly. Additionally, the compounds disclosed herein have advantageous pharmacokinetic properties, biodistribution, and efficiency (e.g., due to the different disassociate rates of the polymer group used).

Compounds of Formula (A′)

Provided herein are compounds which are cationic lipids.

In one aspect, the invention features a cationic lipid having a structure according to Formula (A′),

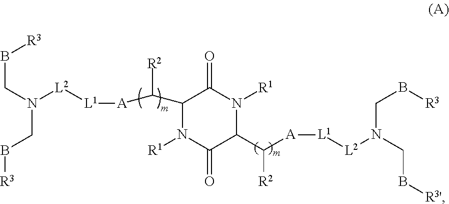

or a pharmaceutically acceptable salt thereof, wherein
each R¹ and R² is independently H or C₁-C₆ aliphatic;
each m is independently an integer having a value of 1 to 4;
each A is independently a covalent bond or arylene;
each L¹ is independently an ester, thioester, disulfide, or anhydride group;
each L² is independently C₂-C₁₀ aliphatic;
each B is independently —CHX¹— or —CH₂CO₂—;
each X¹ is independently H or OH; and
each R³ is independently C₆-C₃₀ aliphatic.

In some embodiments of Formula (A′), each R³ is independently C₆-C₂₀ aliphatic.

Compounds of Formula (A)

In embodiments, the cationic lipids of the present invention include compounds having a structure according to Formula (A),

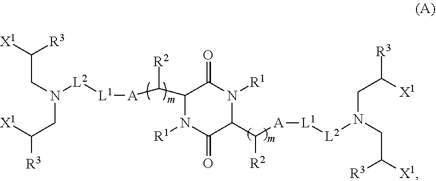

or a pharmaceutically acceptable salt thereof, wherein

each R¹ and R² is independently H or C₁-C₆ aliphatic;

each m is independently an integer having a value of 1 to 4;

each A is independently a covalent bond or arylene;

each L¹ is independently an ester, thioester, disulfide, or anhydride group;

each L² is independently C₂-C₁₀ aliphatic;

each X¹ is independently H or OH; and

each R³ is independently C₆-C₃₀ aliphatic.

In some embodiments of Formula (A), each R³ is independently C₆-C₃₀ aliphatic. In some embodiments of Formula (A), each R³ is independently C₆-C₂₀ aliphatic.

In embodiments of Formula (A′) or Formula (A), R¹ is independently H. In embodiments, R¹ is independently C₁-C₆ aliphatic (e.g., methyl).

In embodiments of Formula (A′) or Formula (A), R² is independently H. In embodiments, R² is independently C₁-C₆ aliphatic (e.g., methyl).

In embodiments of Formula (A′) or Formula (A), m is 1. In embodiments, m is 2. In embodiments, m is 3. In embodiments, m is 4. In embodiments, each m is 1. In embodiments, each m is 2. In embodiments, each m is 3. In embodiments, each m is 4.

In embodiments of Formula (A′) or Formula (A), each A is a covalent bond. In embodiments, each A is arylene.

In embodiments of Formula (A′) or Formula (A), L¹ is independently an ester. In embodiments, L¹ is independently a thioester. In embodiments, L¹ is independently a disulfide. In embodiments, L¹ is independently an anhydride group. In embodiments, each Cis an ester. In embodiments, each L¹ is a thioester. In embodiments, each L¹ is a disulfide. In embodiments, each L¹ is an anhydride group.

In embodiments of Formula (A′) or Formula (A), each L² is C₂ aliphatic (e.g., C₂ alkylene). In embodiments, each L² is C₃ aliphatic (e.g., C₃ alkylene). In embodiments, each L² is C₄ aliphatic (e.g., C₄ alkylene). In embodiments, each L² is C₅ aliphatic (e.g., C₅ alkylene). In embodiments, each L² is C₆ aliphatic (e.g., C₆ alkylene). In embodiments, each L² is C₇ aliphatic (e.g., C₇ alkylene). In embodiments, each L² is C₈ aliphatic (e.g., C₈ alkylene). In embodiments, each L² is C₉ aliphatic (e.g., C₉ alkylene). In embodiments, each L² is C₁₀ aliphatic (e.g., C₁₀ alkylene).

In embodiments of Formula (A′) or Formula (A), X¹ is independently H. In embodiments, X¹ is independently OH. In embodiments, each X¹ is H. In embodiments, each X¹ is OH.

In embodiments of Formula (A′) or Formula (A), each R³ is C₆ aliphatic (e.g., C₆ alkyl or C₆ alkenyl). In embodiments, each R³ is C₇ aliphatic (e.g., C₇ alkyl or C₇ alkenyl). In embodiments, each R³ is C₈ aliphatic (e.g., C₈ alkyl or C₈ alkenyl). In embodiments, each R³ is C₉ aliphatic (e.g., C₉ alkyl or C₉ alkenyl). In embodiments, each R³ is C₁₀ aliphatic (e.g., C₁₀ alkyl or C₁₀ alkenyl). In embodiments, each R³ is C₁₁ aliphatic (e.g., C₁₁ alkyl or C₁₁ alkenyl). In embodiments, each R³ is C₁₂ aliphatic (e.g., C₁₂ alkyl or C₁₂ alkenyl). In embodiments, each R³ is C₁₃ aliphatic (e.g., C₁₃ alkyl or C₁₃ alkenyl). In embodiments, each R³ is C₁₄ aliphatic (e.g., C₁₄ alkyl or C₁₄ alkenyl). In embodiments, each R³ is C₁₅ aliphatic (e.g., C₁₅ alkyl or C₁₅ alkenyl). In embodiments, each R³ is C₁₆ aliphatic (e.g., C₁₆ alkyl or C₁₆ alkenyl). In embodiments, each R³ is C₁₇ aliphatic (e.g., C₁₇ alkyl or C₁₇ alkenyl). In embodiments, each R³ is C₁₈ aliphatic (e.g., C₁₈ alkyl or C₁₈ alkenyl). In embodiments, each R³ is C₁₉ aliphatic (e.g., C₁₉ alkyl or C₁₉ alkenyl). In embodiments, each R³ is C₂₀ aliphatic (e.g., C₂₀ alkyl or C₂₀ alkenyl). In embodiments, R³ is unsubstituted.

In embodiments of Formula (A′) or Formula (A), each R³ is C₂₁ aliphatic (e.g., C₂₁ alkyl or C₂₁ alkenyl). In embodiments, each R³ is C₂₂ aliphatic (e.g., C₂₂ alkyl or C₂₂ alkenyl). In embodiments, each R³ is C₂₃ aliphatic (e.g., C₂₃ alkyl or C₂₃ alkenyl). In embodiments, each R³ is C₂₄ aliphatic (e.g., C₂₄ alkyl or C₂₄ alkenyl). In embodiments, each R³ is C₂₈ aliphatic (e.g., C₂₅ alkyl or C₂₅ alkenyl). In embodiments, each R³ is C₂₆ aliphatic (e.g., C₂₆ alkyl or C₂₆ alkenyl). In embodiments, each R³ is C₂₇ aliphatic (e.g., C₂₇ alkyl or C₂₇ alkenyl). In embodiments, each R³ is C₂₈ aliphatic (e.g., C₂₈ alkyl or C₂₈ alkenyl). In embodiments, each R³ is C₂₉ aliphatic (e.g., C₂₉ alkyl or C₂₉ alkenyl). In embodiments, each R³ is C₃₀ aliphatic (e.g., C₃₀ alkyl or C₃₀ alkenyl).

Compounds of Formula (I)

In embodiments, the cationic lipid of Formula (A) has a structure according to Formula (I),

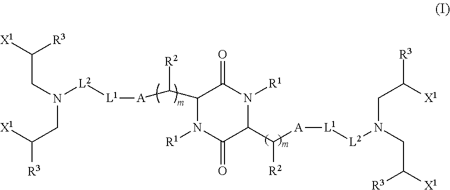

or a pharmaceutically acceptable salt thereof.

In some embodiments of Formula (I), each R³ is independently C₆-C₃₀ aliphatic. In some embodiments of Formula (I), each R³ is independently C₆-C₂₀ aliphatic.

In Formula (I), R¹, R², R³, X¹, L¹, L², and m can be according to any permitted group or value described herein for Formula (A′) or Formula (A). In some embodiments of Formula (I), R¹, R², R³, X¹, L¹, L², and m can be according to any permitted group or value described herein for Formula (A).

In embodiments, the cationic lipid has a structure according to Formula (A′), (A), or (I), wherein each R¹ is H.

In embodiments, the cationic lipid has a structure according to Formula (A′), (A), or (I), wherein each R² is independently H or C₁-C₆ alkyl.

In embodiments, the cationic lipid has a structure according to Formula (A′), (A), or (I), wherein each L² is independently C₂-C₁₀ alkylene.

In embodiments, the cationic lipid has a structure according to Formula (A′), (A), or (I), wherein each R³ is independently C₆-C₂₀ alkyl, C₆-C₂₀ alkenyl, or C₆-C₂₀ alkynyl.

In embodiments, the cationic lipid has a structure according to Formula (A′), (A), or (I), wherein each X¹ is OH.

In embodiments, the cationic lipid has a structure according to Formula (A′), (A), or (I), wherein each m is 1.

In embodiments, the cationic lipid has a structure according to Formula (A′), (A), or (I), wherein each m is 2.

In embodiments, the cationic lipid has a structure according to Formula (A′), (A), or (I), wherein each m is 3.

In embodiments, the cationic lipid has a structure according to Formula (A′), (A), or (I), wherein each m is 4.

Compounds of Formula (I-a)

In embodiments, the cationic lipid of Formula (I) has a structure according to Formula (I-a),

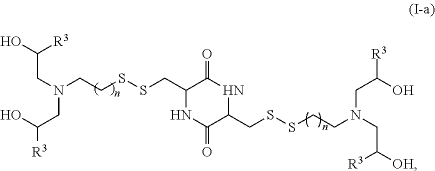

or a pharmaceutically acceptable salt thereof, wherein each n is independently an integer having a value from 1 to 9.

In some embodiments of Formula (I-a), each R³ is independently C₆-C₃₀ aliphatic. In some embodiments of Formula (I-a), each R³ is independently C₆-C₂₀ aliphatic.

In embodiments, the cationic lipid of Formula (I-a) has a structure according to Formula (I-a′),

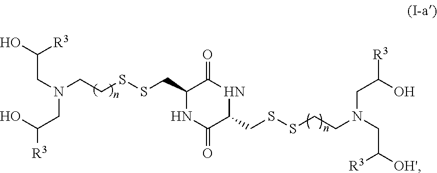

or a pharmaceutically acceptable salt thereof.

In some embodiments of Formula (I-a′), each R³ is independently C₆-C₃₀ aliphatic. In some embodiments of Formula (I-a′), each R³ is independently C₆-C₂₀ aliphatic.

In Formula (I-a) and (I-a′), R³ can be according to any permitted group described herein (e.g., as described for Formula (A′), Formula (A) or Formula (I)). In some embodiments of Formula (I-a) and (I-a′), R³ can be according to any permitted group described for Formula (A) or Formula (I).

In embodiments, the cationic lipid has a structure according to Formula (I-a) or (I-a′), wherein each n is 3. In embodiments, each n is 1. In embodiments, each n is 2. In embodiments, each n is 4. In embodiments, each n is 5. In embodiments, each n is 6. In embodiments, each n is 7. In embodiments, each n is 8. In embodiments, each n is 9.

Compounds of Formula (I-b)

In embodiments, the cationic lipid of Formula (I) has a structure according to Formula (I-b),

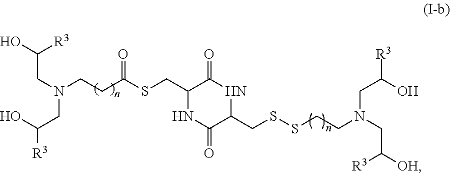

or a pharmaceutically acceptable salt thereof, wherein each n is an integer having a value of 1 to 9.

In some embodiments of Formula (I-b), each R³ is independently C₆-C₃₀ aliphatic. In some embodiments of Formula (I-b), each R³ is independently C₆-C₂₀ aliphatic.

In embodiments, the cationic lipid of Formula (I-b) has a structure according to Formula (I-b′),

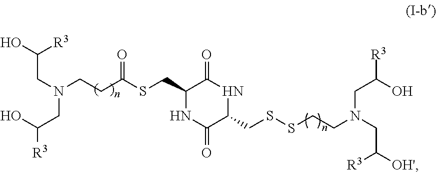

or a pharmaceutically acceptable salt thereof.

In some embodiments of Formula (I-b′), each R³ is independently C₆-C₃₀ aliphatic. In some embodiments of Formula (I-b′), each R³ is independently C₆-C₂₀ aliphatic.

In Formula (I-b) and (I-b′), R³ can be according to any permitted group described herein (e.g., as described for Formula (A′), Formula (A) or Formula (I)). In some embodiments of Formula (I-b) and (I-b′), R³ can be according to any permitted group described for Formula (A) or Formula (I).

In embodiments, the cationic lipid has a structure according to Formula (I-b) or (I-b′), wherein each n is 2. In embodiments, each n is 1. In embodiments, each n is 3. In embodiments, each n is 4. In embodiments, each n is 5. In embodiments, each n is 6. In embodiments, each n is 7. In embodiments, each n is 8. In embodiments, each n is 9.

Compounds of Formula (I-c)

In embodiments, the cationic lipid of Formula (I) has a structure according to Formula (I-c),

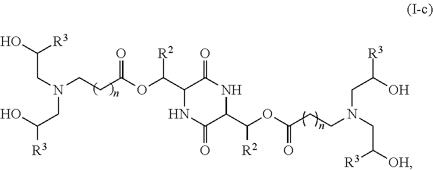

or a pharmaceutically acceptable salt thereof, wherein
each n is an integer having a value of 1 to 9; and
each R² is independently H or CH₃.

In some embodiments of Formula (I-c), each R³ is independently C₆-C₃₀ aliphatic. In some embodiments of Formula (I-c), each R³ is independently C₆-C₂₀ aliphatic.

In embodiments, the cationic lipid of Formula (I-c) has a structure according to Formula (I-c′),

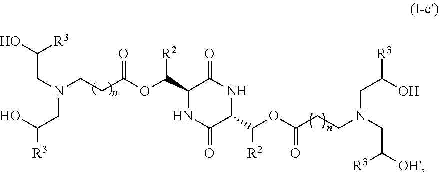

or a pharmaceutically acceptable salt thereof

In some embodiments of Formula (I-c′), each R³ is independently C₆-C₃₀ aliphatic. In some embodiments of Formula (I-c′), each R³ is independently C₆-C₂₀ aliphatic.

In Formula (I-c) and (I-c′), each of R² and R³ can independently be according to any permitted group described herein (e.g., as described for Formula (A′), Formula (A), or Formula (I)). In some embodiments of Formula (I-c) and (I-c′), each of R² and R³ can be according to any permitted group described for Formula (A) or Formula (I).

In embodiments, the cationic lipid has a structure according to Formula (I-c-1),

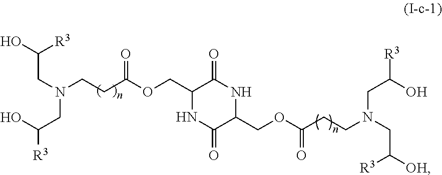

or a pharmaceutically acceptable salt thereof.

In some embodiments of Formula (I-c-1), each R³ is independently C₆-C₃₀ aliphatic. In some embodiments of Formula (I-c-1), each R³ is independently C₆-C₂₀ aliphatic.

In Formula (I-c-1), each R³ can independently be according to any permitted group described herein (e.g., as described for Formula (A′), Formula (A) or Formula (I)). In some embodiments of Formula (I-c-1), each R³ can independently be according to any permitted group described herein for Formula (A) or Formula (I).

In embodiments, the cationic lipid has a structure according to Formula (I-c′-1),

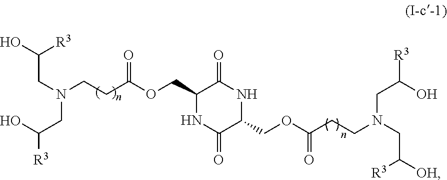

or a pharmaceutically acceptable salt thereof.

In some embodiments of Formula (I-c′-1), each R³ is independently C₆-C₃₀ aliphatic. In some embodiments of Formula (I-c′-1), each R³ is independently C₆-C₂₀ aliphatic.

In Formula (I-c′-1), each R³ can independently be according to any permitted group described herein (e.g., as described for Formula (A′), Formula (A) or Formula (I)). In some embodiments of Formula (I-c′-1), each R³ can independently be according to any permitted group described herein for Formula (A) or Formula (I).

In embodiments, the cationic lipid has a structure according to Formula (I-c) or (I-c′), wherein each R² is CH₃.

In embodiments, the cationic lipid has a structure according to Formula (I-c-2),

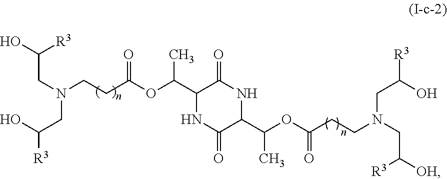

or a pharmaceutically acceptable salt thereof.

In some embodiments of Formula (I-c-2), each R³ is independently C₆-C₃₀ aliphatic. In some embodiments of Formula (I-c-2), each R³ is independently C₆-C₂₀ aliphatic.

In Formula (I-c-2), each R³ can independently be according to any permitted group described herein (e.g., as described for Formula (A′), Formula (A), or Formula (I)). In some embodiments of Formula (I-c-2), each R³ can independently be according to any permitted group described herein for Formula (A) or Formula (I).

In embodiments, the cationic lipid has a structure according to Formula (I-c′-2),

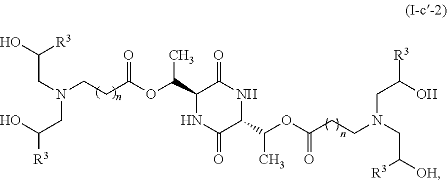

or a pharmaceutically acceptable salt thereof.

In some embodiments of Formula (I-c′-2), each R³ is independently C₆-C₃₀ aliphatic. In some embodiments of Formula (I-c′-2), each R³ is independently C₆-C₂₀ aliphatic.

In Formula (I-c′-2), each R³ can independently be according to any permitted group described herein (e.g., as described for Formula (A′), Formula (A), or Formula (I)). In some embodiments of Formula (I-c′-2), each R³ can independently be according to any permitted group described herein for Formula (A) or Formula (I).

In embodiments, the cationic lipid has a structure according to Formula (I-c) or (I-c′) (e.g., according to Formula (I-c-1), (I-c′-1), (I-c-2), or (I-c′-2)), wherein each n is 2. In embodiments, each n is 1. In embodiments, each n is 3. In embodiments, each n is 4. In embodiments, each n is 5. In embodiments, each n is 6. In embodiments, each n is 7. In embodiments, each n is 8. In embodiments, each n is 9.

In embodiments, the cationic lipid has a structure according to Formula (I-c) or (I-c′) (e.g., according to Formula (I-c-1), (I-c′-1), (I-c-2), or (I-c′-2)), wherein each R² is H.

Compounds of Formula (I-d)

In embodiments, the cationic lipid of Formula (I) has a structure according to Formula (I-d),

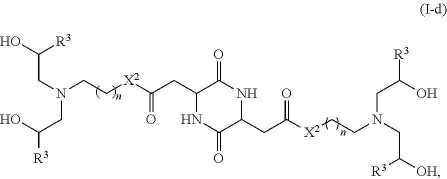

or a pharmaceutically acceptable salt thereof, wherein
each n is independently an integer having a value of 1 to 9; and
each X² is independently 0 or S.

In some embodiments of Formula (I-d), each R³ is independently C₆-C₃₀ aliphatic. In some embodiments of Formula (I-d), each R³ is independently C₆-C₂₀ aliphatic.

In embodiments, the cationic lipid of Formula (I-d) has a structure according to Formula (I-d′),

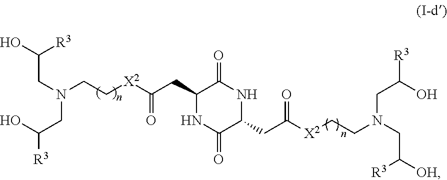

or a pharmaceutically acceptable salt thereof

In some embodiments of Formula (I-d′), each R³ is independently C₆-C₃₀ aliphatic. In some embodiments of Formula (I-d′), each R³ is independently C₆-C₂₀ aliphatic.

In embodiments, the cationic lipid has a structure according to Formula (I-d) or (I-d′), wherein each X² is S.

In embodiments, the cationic lipid has a structure according to Formula (I-d-1),

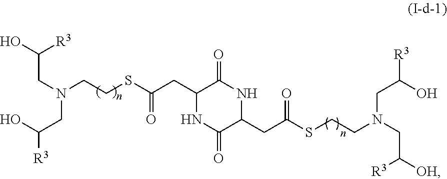

or a pharmaceutically acceptable salt thereof.

In some embodiments of Formula (I-d-1), each R³ is independently C₆-C₃₀ aliphatic. In some embodiments of Formula (I-d-1), each R³ is independently C₆-C₂₀ aliphatic.

In embodiments, the cationic lipid has a structure according to Formula (I-d) or (I-d′), wherein each X² is O.

In embodiments, the cationic lipid has a structure according to Formula (I-d-2),

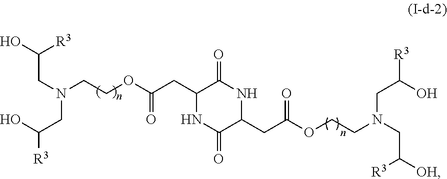

or a pharmaceutically acceptable salt thereof.

In some embodiments of Formula (I-d-2), each R³ is independently C₆-C₃₀ aliphatic. In some embodiments of Formula (I-d-2), each R³ is independently C₆-C₂₀ aliphatic.

In any of Formulas (I-d), (I-d′), (I-d-1), and (I-d-2), R³ can be according to any permitted group described herein (e.g., as described for Formula (A′), Formula (A) or Formula (I)). In some embodiments of any of Formulas (I-d), (I-d′), (I-d-1), and (I-d-2), R³ can be according to any permitted group described herein for Formula (A) or Formula (I).

In embodiments, the cationic lipid has a structure according to any of Formulas (I-d), (I-d′), (I-d-1), and (I-d-2), wherein each n is 3. In embodiments, each n is 1. In embodiments, each n is 2. In embodiments, each n is 4. In embodiments, each n is 5. In embodiments, each n is 6. In embodiments, each n is 7. In embodiments, each n is 8. In embodiments, each n is 9.

Compounds of Formula (I-e)

In embodiments, the cationic lipid of Formula (I) has a structure according to Formula (I-e),

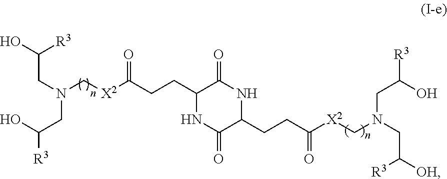

or a pharmaceutically acceptable salt thereof, wherein
each n is independently an integer of having a value of 2 to 10; and
each X² is independently 0 or S.

In some embodiments of Formula (I-e), each R³ is independently C₆-C₃₀ aliphatic. In some embodiments of Formula (I-e), each R³ is independently C₆-C₂₀ aliphatic.

In embodiments, the cationic lipid of Formula (I-e) has a structure according to Formula (I-e′),

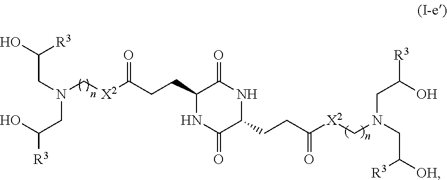

or a pharmaceutically acceptable salt thereof.

In some embodiments of Formula (I-e′), each R³ is independently C₆-C₃₀ aliphatic. In some embodiments of Formula (I-e′), each R³ is independently C₆-C₂₀ aliphatic.

In embodiments, the cationic lipid has a structure according to Formula (I-e) or (I-e′), wherein each X² is S.

In embodiments, the cationic lipid has a structure according to Formula (I-e-1),

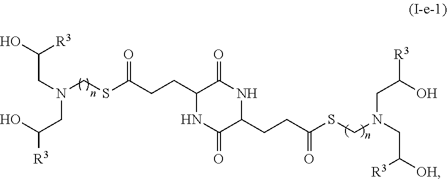

or a pharmaceutically acceptable salt thereof.

In some embodiments of Formula (I-e-1), each R³ is independently C₆-C₃₀ aliphatic. In some embodiments of Formula (I-e-1), each R³ is independently C₆-C₂₀ aliphatic.

In embodiments, the cationic lipid has a structure according to Formula (I-e) or (I-e′), wherein each X² is O.

In embodiments, the cationic lipid has a structure according to Formula (I-e-2),

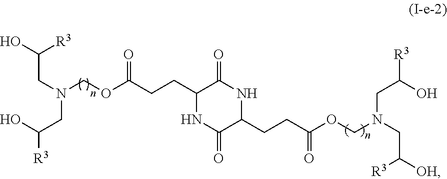

or a pharmaceutically acceptable salt thereof.

In some embodiments of Formula (I-e-2), each R³ is independently C₆-C₃₀ aliphatic. In some embodiments of Formula (I-e-2), each R³ is independently C₆-C₂₀ aliphatic.

In any of Formulas (I-e), (I-e′), (I-e-1), and (I-e-2), R³ can be according to any permitted group described herein (e.g., as described for Formula (A′), Formula (A) or Formula (I)). In some embodiments of any of Formulas (I-e), (I-e′), (I-e-1), and (I-e-2), R³ can be according to any permitted group described herein for Formula (A) or Formula (I).

In embodiments, the cationic lipid has a structure according to any of Formulas (I-e), (I-e′), (I-e-1), and (I-e-2), wherein each n is 4. In embodiments, each n is 2. In embodiments, each n is 3. In embodiments, each n is 5. In embodiments, each n is 6. In embodiments, each n is 7. In embodiments, each n is 8. In embodiments, each n is 9. In embodiments, each n is 10.

Compounds of Formula (I-f)

In embodiments, the cationic lipid has a structure according to Formula (I-f),

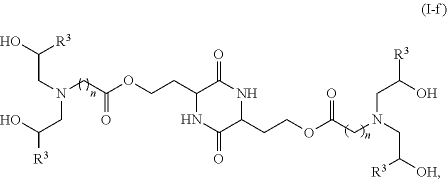

or a pharmaceutically acceptable salt thereof, wherein

each n is independently an integer of having a value of 2 to 10.

In some embodiments of Formula (I-f), each R³ is independently C₆-C₃₀ aliphatic. In some embodiments of Formula (I-f), each R³ is independently C₆-C₂₀ aliphatic.

In embodiments, the cationic lipid has a structure according to Formula (I-f′),

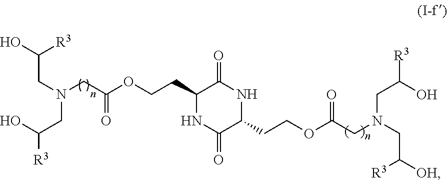

or a pharmaceutically acceptable salt thereof.

In some embodiments of Formula (I-f′), each R³ is independently C₆-C₃₀ aliphatic. In some embodiments of Formula (I-f′), each R³ is independently C₆-C₂₀ aliphatic.

In Formula (I-f) and (I-f′), R³ can be according to any permitted group described herein (e.g., as described for Formula (A′), Formula (A) or Formula (I)). In some embodiments of Formula (I-f) and (I-f′), R³ can be according to any permitted group described herein for Formula (A) or Formula (I).

In embodiments, the cationic lipid has a structure according to Formula (I-f) or (I-f′), wherein each n is 3. In embodiments, each n is 2. In embodiments, each n is 4. In embodiments, each n is 5. In embodiments, each n is 6. In embodiments, each n is 7. In embodiments, each n is 8. In embodiments, each n is 9. In embodiments, each n is 10.

Compounds of Formula (II)

In embodiments, the cationic lipid of Formula (A) has a structure according to Formula (II),

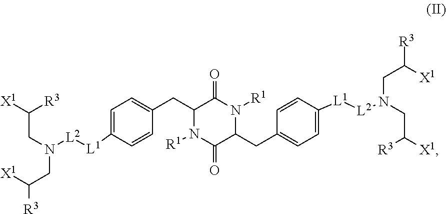

or a pharmaceutically acceptable salt thereof, wherein
each R¹ is independently H or C₁-C₆ aliphatic;
each L¹ is independently an ester, thioester, disulfide, or anhydride group;
each L² is independently C₂-C₁₀ aliphatic;
each X¹ is independently H or OH; and
each R³ is independently C₆-C₃₀ aliphatic.

In some embodiments of Formula (II), each R³ is independently C₆-C₂₀ aliphatic. In some embodiments of Formula (II), each R³ is independently C₈-C₂₀ aliphatic.

In embodiments, the cationic lipid has a structure according to Formula (II), wherein R¹ is independently H. In embodiments, the cationic lipid has a structure according to Formula (II), wherein R¹ is independently C₁-C₆ aliphatic (e.g., methyl). In embodiments, the cationic lipid has a structure according to Formula (II), wherein each R¹ is independently H or C₁-C₆ alkyl. In embodiments, the cationic lipid has a structure according to Formula (II), wherein each R¹ is H.

In embodiments, the cationic lipid has a structure according to Formula (II), wherein L¹ is independently an ester. In embodiments, the cationic lipid has a structure according to Formula (II), wherein L¹ is independently a thioester. In embodiments, the cationic lipid has a structure according to Formula (II), wherein L¹ is independently a disulfide. In embodiments, the cationic lipid has a structure according to Formula (II), wherein L¹ is independently an anhydride group. In embodiments, the cationic lipid has a structure according to Formula (II), wherein each L¹ is an ester. In embodiments, each L¹ is a thioester. In embodiments, the cationic lipid has a structure according to Formula (II), wherein each L¹ is a disulfide. In embodiments, the cationic lipid has a structure according to Formula (II), wherein each L¹ is an anhydride group.

In embodiments, the cationic lipid has a structure according to Formula (II), wherein each L² is C₂ aliphatic (e.g., C₂ alkylene). In embodiments, the cationic lipid has a structure according to Formula (II), wherein each L² is C₃ aliphatic (e.g., C₃ alkylene). In embodiments, the cationic lipid has a structure according to Formula (II), wherein each L² is C₄ aliphatic (e.g., C₄ alkylene). In embodiments, the cationic lipid has a structure according to Formula (II), wherein each L² is C₅ aliphatic (e.g., C₅ alkylene). In embodiments, the cationic lipid has a structure according to Formula (II), wherein each L² is C₆ aliphatic (e.g., C₆ alkylene). In embodiments, the cationic lipid has a structure according to Formula (II), wherein each L² is C₇ aliphatic (e.g., C₇ alkylene). In embodiments, the cationic lipid has a structure according to Formula (II), wherein each L² is C₈ aliphatic (e.g., C₈ alkylene). In embodiments, the cationic lipid has a structure according to Formula (II), wherein each L² is C₉ aliphatic (e.g., C₉ alkylene). In embodiments, each L² is C₁₀ aliphatic (e.g., C₁₀ alkylene).

In embodiments, the cationic lipid has a structure according to Formula (II), wherein X¹ is independently H. In embodiments, the cationic lipid has a structure according to Formula (II), wherein X¹ is independently OH. In embodiments, the cationic lipid has a structure according to Formula (II), wherein each X¹ is H. In embodiments, the cationic lipid has a structure according to Formula (II), wherein each X¹ is OH.

In embodiments, the cationic lipid has a structure according to Formula (II), wherein each R³ is C₈ aliphatic (e.g., C₈ alkyl or C₈ alkenyl). In embodiments, the cationic lipid has a structure according to Formula (II), wherein each R³ is C₉ aliphatic (e.g., C₉ alkyl or C₉ alkenyl). In embodiments, the cationic lipid has a structure according to Formula (II), wherein each R³ is C₁₀ aliphatic (e.g., C₁₀ alkyl or C₁₀ alkenyl). In embodiments, the cationic lipid has a structure according to Formula (II), wherein each R³ is C₁₁ aliphatic (e.g., C₁₁ alkyl or C₁₁ alkenyl). In embodiments, the cationic lipid has a structure according to Formula (II), wherein each R³ is C₁₂ aliphatic (e.g., C₁₂ alkyl or C₁₂ alkenyl). In embodiments, the cationic lipid has a structure according to Formula (II), wherein each R³ is C₁₃ aliphatic (e.g., C₁₃ alkyl or C₁₃ alkenyl). In embodiments, the cationic lipid has a structure according to Formula (II), wherein each R³ is C₁₄ aliphatic (e.g., C₁₄ alkyl or C₁₄ alkenyl). In embodiments, the cationic lipid has a structure according to Formula (II), wherein each R³ is C₁₅ aliphatic (e.g., C₁₅ alkyl or C₁₅ alkenyl). In embodiments, the cationic lipid has a structure according to Formula (II), wherein each R³ is C₁₆ aliphatic (e.g., C₁₆ alkyl or C₁₆ alkenyl). In embodiments, the cationic lipid has a structure according to Formula (II), wherein each R³ is C₁₇ aliphatic (e.g., C₁₇ alkyl or C₁₇ alkenyl). In embodiments, the cationic lipid has a structure according to Formula (II), wherein each R³ is C₁₈ aliphatic (e.g., C₁₈ alkyl or C₁₈ alkenyl). In embodiments, the cationic lipid has a structure according to Formula (II), wherein each R³ is C₁₉ aliphatic (e.g., C₁₉ alkyl or C₁₉ alkenyl). In embodiments, the cationic lipid has a structure according to Formula (II), wherein each R³ is C₂₀ aliphatic (e.g., C₂₀ alkyl or C₂₀ alkenyl). In embodiments, the cationic lipid has a structure according to Formula (II), wherein R³ is unsubstituted.

In embodiments of Formula (II), each R³ is C₂₁ aliphatic (e.g., C₂₁ alkyl or C₂₁ alkenyl). In embodiments, each R³ is C₂₂ aliphatic (e.g., C₂₂ alkyl or C₂₂ alkenyl). In embodiments, each R³ is C₂₃ aliphatic (e.g., C₂₃ alkyl or C₂₃ alkenyl). In embodiments, each R³ is C₂₄ aliphatic (e.g., C₂₄ alkyl or C₂₄ alkenyl). In embodiments, each R³ is C₂₅ aliphatic (e.g., C₂₅ alkyl or C₂₅ alkenyl). In embodiments, each R³ is C₂₆ aliphatic (e.g., C₂₆ alkyl or C₂₆ alkenyl). In embodiments, each R³ is C₂₇ aliphatic (e.g., C₂₇ alkyl or C₂₇ alkenyl). In embodiments, each R³ is C₂₈ aliphatic (e.g., C₂₈ alkyl or C₂₈ alkenyl). In embodiments, each R³ is C₂₉ aliphatic (e.g., C₂₉ alkyl or C₂₉ alkenyl). In embodiments, each R³ is C₃₀ aliphatic (e.g., C₃₀ alkyl or C₃₀ alkenyl).

Compounds of Formula (II-a)

In embodiments, the cationic lipid of Formula (II) has a structure according to Formula (II-a),

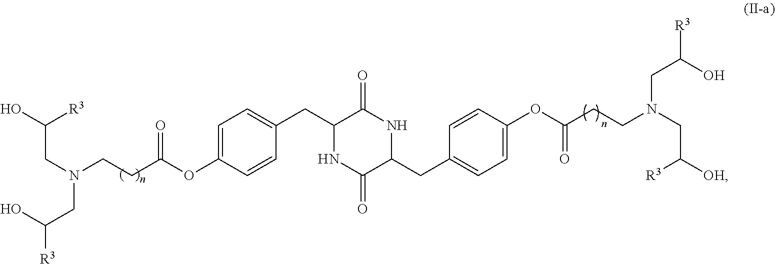

or a pharmaceutically acceptable salt thereof, wherein each n is an integer having a value of 1 to 9.

In some embodiments of Formula (II-a), each R³ is independently C₆-C₃₀ aliphatic. In some embodiments of Formula (II-a), each R³ is independently C₆-C₂₀ aliphatic. In some embodiments of Formula (II-a), each R³ is independently C₈-C₂₀ aliphatic.

In embodiments, the cationic lipid of Formula (II-a) has a structure according to Formula (II-a′),

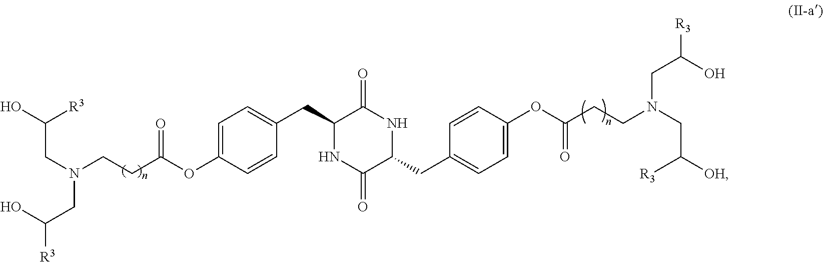

or a pharmaceutically acceptable salt thereof.

In some embodiments of Formula (II-a′), each R³ is independently C₆-C₃₀ aliphatic. In some embodiments of Formula (II-a′), each R³ is independently C₆-C₂₀ aliphatic. In some embodiments of Formula (II-a′), each R³ is independently C₈-C₂₀ aliphatic.

In Formula (II-a) and (II-a′), R³ can be according to any permitted group described herein for Formula (A′), Formula (A) or Formula (II). In some embodiments of Formula (II-a) and (II-a′), R³ can be according to any permitted group described herein for Formula (A) or Formula (II).

In embodiments, the cationic lipid has a structure according to Formula (II-a) or (II-a′), wherein each n is 2. In embodiments, each n is 1. In embodiments, each n is 3. In embodiments, each n is 4. In embodiments, each n is 5. In embodiments, each n is 6. In embodiments, each n is 7. In embodiments, each n is 8. In embodiments, each n is 9.

Compounds of Formulas (III) and (III′)

In embodiments, the cationic lipid of Formula (A′) has a structure according to Formula (III),

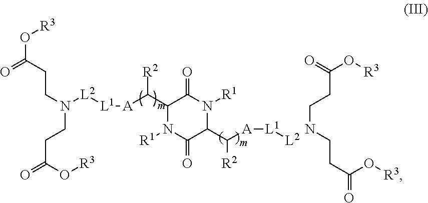

or a pharmaceutically acceptable salt thereof, wherein
each R¹ and R² is independently H or C₁-C₆ aliphatic;
each m is independently an integer having a value of 1 to 4;
each A is independently a covalent bond or arylene;
each L¹ is independently an ester, thioester, disulfide, or anhydride group;
each L² is independently C₂-C₁₀ aliphatic;
each R³ is independently C₆-C₃₀ aliphatic.

In some embodiments of Formula (III), each R³ is independently C₆-C₂₀ aliphatic.

In Formula (III), each R¹, R², m, A, L¹, L², and R³ can independently be according to any permitted group recited in any aspect or embodiment described herein (e.g., as described for Formula (A′), (A), or Formula (I)).

In embodiments, each A is independently a covalent bond or phenylene.

In embodiments, the cationic lipid of Formula (III) has the following structure,

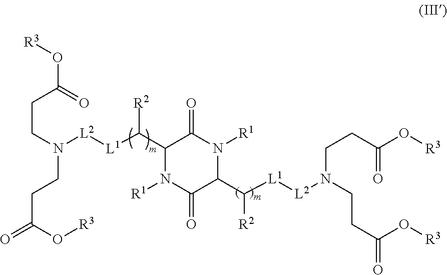

or a pharmaceutically acceptable salt thereof.

In some embodiments of Formula (III′), each R³ is independently C₆-C₃₀ aliphatic. In some embodiments of Formula (III′), each R³ is independently C₆-C₂₀ aliphatic.

In Formula (III′), each R¹, R², m, L¹, L², and R³ can independently be according to any permitted group recited in any aspect or embodiment described herein (e.g., as described for Formula (A′), (A), or Formula (III)).

In embodiments of Formula (III) or (III′), each R¹ is H.

In embodiments of Formula (III) or (III′), each R² is independently H or C₁-C₆ alkyl.

In embodiments of Formula (III) or (III′), each L² is independently C₂-C₁₀ alkylene.

In embodiments of Formula (III) or (III′), each R³ is independently C₆-C₂₀ alkyl, C₆-C₂₀ alkenyl, or C₆-C₂₀ alkynyl. In embodiments, R³ comprises a substituent that is —O—C(O)R′ or —C(O)—OR′, wherein R′ is C₁-C₁₆ alkyl.

In embodiments of Formula (III) or (III′), each m is 1. In embodiments, each m is 2. In embodiments, each m is 3. In embodiments, each m is 4.

Compounds of Formula (III-a)

In embodiments, the cationic lipid of Formula (III) has the following structure:

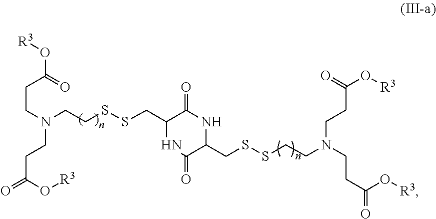

or a pharmaceutically acceptable salt thereof, wherein each n is independently an integer having a value from 1 to 9.

In some embodiments of Formula (III-a), each R³ is independently C₆-C₃₀ aliphatic. In some embodiments of Formula (III-a), each R³ is independently C₆-C₂₀ aliphatic.

In embodiments, the cationic lipid of Formula (III) or (III-a) has the following structure:

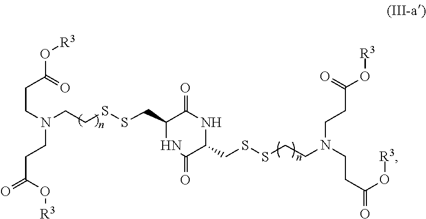

or a pharmaceutically acceptable salt thereof.

In some embodiments of Formula (III-a′), each R³ is independently C₆-C₃₀ aliphatic. In some embodiments of Formula (III-a′), each R³ is independently C₆-C₂₀ aliphatic.

In Formula (III-a) or (III-a′), each R³ can independently be according to any permitted group described herein (e.g., as described for Formula (A′), Formula (A) or Formula (III)).

In embodiments of Formula (III-a) or (III-a′), each n is 3. In embodiments, each n is 1. In embodiments, each n is 2. In embodiments, each n is 4. In embodiments, each n is 5. In embodiments, each n is 6. In embodiments, each n is 7. In embodiments, each n is 8. In embodiments, each n is 9.

Compounds of Formula (III-b)

In embodiments, the cationic lipid of Formula (III) has the following structure:

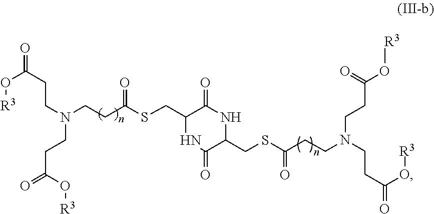

or a pharmaceutically acceptable salt thereof, wherein each n is an integer having a value of 1 to 9.

In some embodiments of Formula (III-b), each R³ is independently C₆-C₃₀ aliphatic. In some embodiments of Formula (III-b), each R³ is independently C₆-C₂₀ aliphatic.

In embodiments, the cationic lipid of Formula (III) or (III-b) has the following structure:

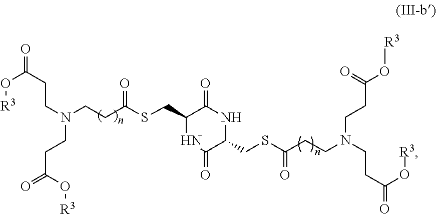

or a pharmaceutically acceptable salt thereof.

In some embodiments of Formula (III-b′), each R³ is independently C₆-C₃₀ aliphatic. In some embodiments of Formula (III-b′), each R³ is independently C₆-C₂₀ aliphatic.

In Formula (III-b) or (III-b′), each R³ can independently be according to any permitted group described herein (e.g., as described for Formula (A′), Formula (A) or Formula (III)).

In embodiments of Formula (III-b) or (III-b′), each n is 2. In embodiments, each n is 1. In embodiments, each n is 3. In embodiments, each n is 4. In embodiments, each n is 5. In embodiments, each n is 6. In embodiments, each n is 7. In embodiments, each n is 8. In embodiments, each n is 9.

Compounds of Formula (III-c)

In embodiments, the cationic lipid of Formula (III) has the following structure:

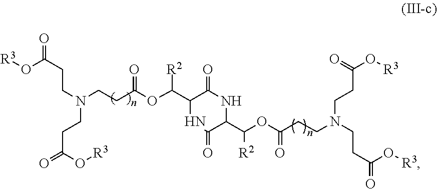

or a pharmaceutically acceptable salt thereof, wherein each n is an integer having a value of 1 to 9; and each R² is independently H or CH₃.

In some embodiments of Formula (III-c), each R³ is independently C₆-C₃₀ aliphatic. In some embodiments of Formula (III-c), each R³ is independently C₆-C₂₀ aliphatic.

In embodiments, the cationic lipid of Formula (III) or Formula (III-c) has the following structure:

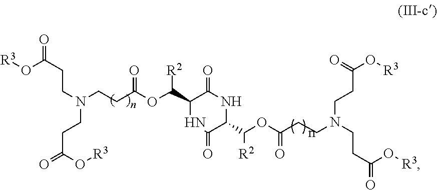

or a pharmaceutically acceptable salt thereof.

In some embodiments of Formula (III-c′), each R³ is independently C₆-C₃₀ aliphatic. In some embodiments of Formula (III-c′), each R³ is independently C₆-C₂₀ aliphatic.

In embodiments of Formula (III-c) or Formula (III-c′), each R² is H.

In embodiments, the cationic lipid of Formula (III) or Formula (III-c) has the following structure:

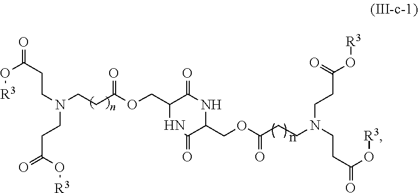

or a pharmaceutically acceptable salt thereof.

In some embodiments of Formula (III-c-1), each R³ is independently C₆-C₃₀ aliphatic. In some embodiments of Formula (III-c-1), each R³ is independently C₆-C₂₀ aliphatic.

In embodiments, the cationic lipid of Formula (III), Formula (III-c), Formula (III-c′) or Formula (III-c-1) has, a cationic lipid has the following structure:

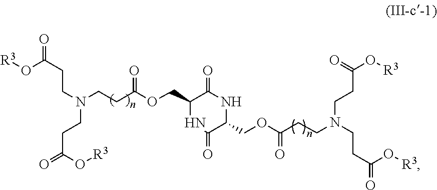

or a pharmaceutically acceptable salt thereof.

In some embodiments of Formula (III-c′-1), each R³ is independently C₆-C₃₀ aliphatic. In some embodiments of Formula (III-c′-1), each R³ is independently C₆-C₂₀ aliphatic.

In embodiments of Formula (III-c) or Formula (III-c′), each R² is CH₃.

In embodiments, the cationic lipid of Formula (III) or Formula (III-c) has the following structure:

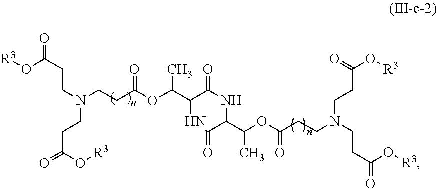

or a pharmaceutically acceptable salt thereof.

In some embodiments of Formula (III-c-2), each R³ is independently C₆-C₃₀ aliphatic. In some embodiments of Formula (III-c-2), each R³ is independently C₆-C₂₀ aliphatic.

In embodiments, the cationic lipid of Formula (III), Formula (III-c), Formula (III-c′) or Formula (III-c-2) has the following structure:

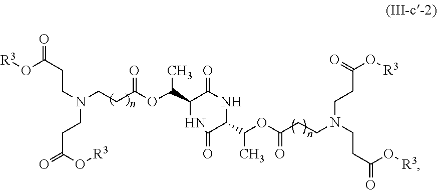

or a pharmaceutically acceptable salt thereof.

In some embodiments of Formula (III-c′-2), each R³ is independently C₆-C₃₀ aliphatic. In some embodiments of Formula (III-c′-2), each R³ is independently C₆-C₂₀ aliphatic.

In embodiments, the cationic lipid has a structure according to Formula (III-c), (III-c′), (III-c-1), (III-c′-1), (III-c-2) or (III-c′-2) wherein each n is 1. In embodiments, the cationic lipid has a structure according to Formula (III-c), (III-c′), (III-c-1), (III-c′-1), (III-c-2) or (III-c′-2), wherein each n is 2. In embodiments, the cationic lipid has a structure according to Formula (III-c), (III-c′), (III-c-1), (III-c′-1), (III-c-2) or (III-c′-2), wherein each n is 3.

In Formula (III-c), (III-c′), (III-c-1), (III-c′-1), (III-c-2), or (III-c′-2), each R³ can independently be according to any permitted group described herein (e.g., as described for Formula (A′), Formula (A) or Formula (III)).

In embodiments of Formula (III-c), (III-c′), (III-c-1), (III-c′-1), (III-c-2), or (III-c′-2), each n is 1. In embodiments, each n is 2. In embodiments, each n is 3. In embodiments, each n is 4. In embodiments, each n is 5. In embodiments, each n is 6. In embodiments, each n is 7. In embodiments, each n is 8. In embodiments, each n is 9.

Compounds of Formula (III-d)

In embodiments, the cationic lipid of Formula (III) has the following structure:

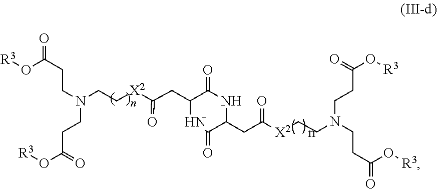

or a pharmaceutically acceptable salt thereof, wherein each n is independently an integer having a value of 1 to 9; and each X² is independently 0 or S.

In some embodiments of Formula (III-d), each R³ is independently C₆-C₃₀ aliphatic. In some embodiments of Formula (III-d), each R³ is independently C₆-C₂₀ aliphatic.

In embodiments, the cationic lipid of Formula (III) or Formula (III-d) has the following structure:

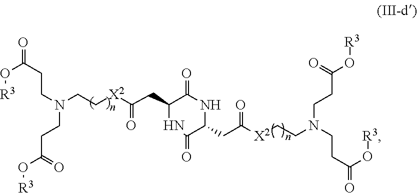

or a pharmaceutically acceptable salt thereof.

In some embodiments of Formula (III-d′), each R³ is independently C₆-C₃₀ aliphatic. In some embodiments of Formula (III-d′), each R³ is independently C₆-C₂₀ aliphatic.

In embodiments, the cationic lipid has a structure according to Formula (III-d) or (III-d′), wherein each n is 1. In embodiments, the cationic lipid has a structure according to Formula (III-d) or (III-d′), wherein each n is 2. In embodiments, the cationic lipid has a structure according to Formula (III-d) or (III-d′), wherein each n is 3. In embodiments, n is 4. In embodiments, n is 5. In embodiments, n is 6. In embodiments, n is 7. In embodiments, n is 8. In embodiments, n is 9.

In embodiments of Formula (III-d) or Formula (III-d′), each X² is S.

In embodiments, the cationic lipid of Formula (III) or Formula (III-d) has the following structure:

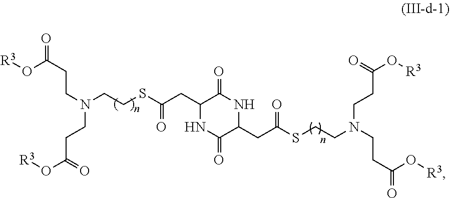

or a pharmaceutically acceptable salt thereof.

In some embodiments of Formula (III-d-1), each R³ is independently C₆-C₃₀ aliphatic. In some embodiments of Formula (III-d-1), each R³ is independently C₆-C₂₀ aliphatic.

In embodiments of Formula (III-d) or Formula (III-d′), each X² is O.

In embodiments, the cationic lipid of Formula (III) or Formula (III-d) has the following structure:

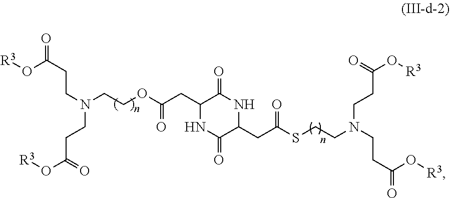

or a pharmaceutically acceptable salt thereof.

In some embodiments of Formula (III-d-2), each R³ is independently C₆-C₃₀ aliphatic. In some embodiments of Formula (III-d-2), each R³ is independently C₆-C₂₀ aliphatic.

In Formula (III-d), (III-d′), (III-d-1), or (III-d-2), each R³ can independently be according to any permitted group described herein (e.g., as described for Formula (A′), Formula (A) or Formula (III)).

In embodiments of Formula (III-d), (III-d′), (III-d-1), or (III-d-2), each n is 1. In embodiments, each n is 2. In embodiments, each n is 3. In embodiments, each n is 4. In embodiments, each n is 5. In embodiments, each n is 6. In embodiments, each n is 7. In embodiments, each n is 8. In embodiments, each n is 9.

Compounds of Formula (III-e)

In embodiments, the cationic lipid of Formula (III) has the following structure:

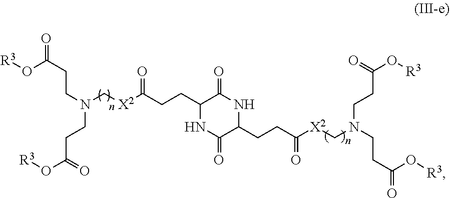

or a pharmaceutically acceptable salt thereof, wherein each n is independently an integer of having a value of 2 to 10; and each X² is independently 0 or S.

In some embodiments of Formula (III-e), each R³ is independently C₆-C₃₀ aliphatic. In some embodiments of Formula (III-e), each R³ is independently C₆-C₂₀ aliphatic.

In embodiments, the cationic lipid of Formula (III) or Formula (III-e) has the following structure:

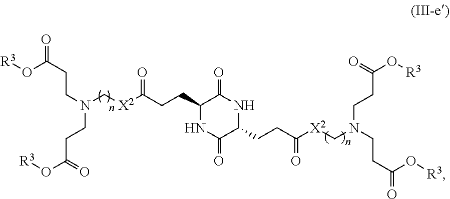

or a pharmaceutically acceptable salt thereof.

In some embodiments of Formula (III-e′), each R³ is independently C₆-C₃₀ aliphatic. In some embodiments of Formula (III-e′), each R³ is independently C₆-C₂₀ aliphatic

In embodiments of Formula (III-e) or Formula (III-e′), each n is 2. In embodiments, each n is 3. In embodiments, each n is 4. In embodiments, each n is 5. In embodiments, each n is 6. In embodiments, each n is 7. In embodiments, each n is 8. In embodiments, each n is 9. In embodiments, each n is 10.

In embodiments of Formula (III-e) or Formula (III-e′), each X² is S.

In embodiments, the cationic lipid of Formula (III) or Formula (III-e) has the following structure:

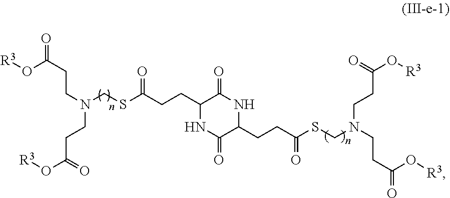

or a pharmaceutically acceptable salt thereof.

In some embodiments of Formula (III-e-1), each R³ is independently C₆-C₃₀ aliphatic. In some embodiments of Formula (III-e-1), each R³ is independently C₆-C₂₀ aliphatic.

In embodiments of Formula (III-e) or Formula (III-e′), each X² is O.

In embodiments, the cationic lipid of Formula (III) or Formula (III-e) has the following structure:

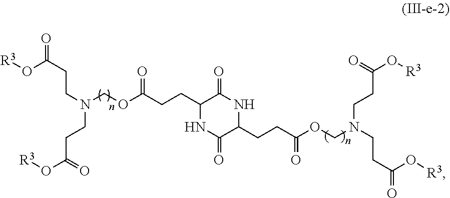

or a pharmaceutically acceptable salt thereof.

In some embodiments of Formula (III-e-2), each R³ is independently C₆-C₃₀ aliphatic. In some embodiments of Formula (III-e-2), each R³ is independently C₆-C₂₀ aliphatic.

In Formula (III-e), (III-e′), (III-e-1), or (III-e-2), each R³ can independently be according to any permitted group described herein (e.g., as described for Formula (A′), Formula (A) or Formula (III)).

In embodiments of Formula (III-e), (III-e′), (III-e-1), or (III-e-2), each n is 2. In embodiments, each n is 3. In embodiments, each n is 4. In embodiments, each n is 5. In embodiments, each n is 6. In embodiments, each n is 7. In embodiments, each n is 8. In embodiments, each n is 9. In embodiments, each n is 10.

Compounds of Formula (III-f)

In embodiments, the cationic lipid of Formula (III) has the following structure:

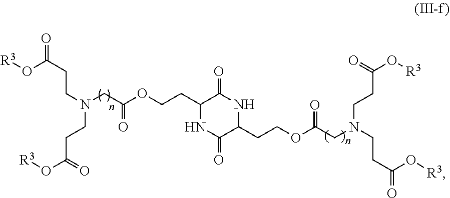

or a pharmaceutically acceptable salt thereof, wherein each n is independently an integer of having a value of 2 to 10.

In some embodiments of Formula (III-f), each R³ is independently C₆-C₃₀ aliphatic. In some embodiments of Formula (III-f), each R³ is independently C₆-C₂₀ aliphatic.

In embodiments, the cationic lipid of Formula (III) or Formula (III-f) has the following structure:

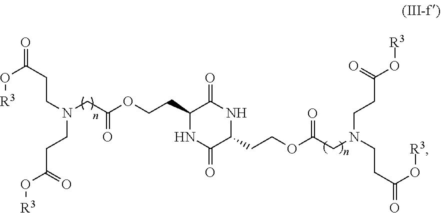

or a pharmaceutically acceptable salt thereof.

In some embodiments of Formula (III-f′), each R³ is independently C₆-C₃₀ aliphatic. In some embodiments of Formula (III-f′), each R³ is independently C₆-C₂₀ aliphatic.

In embodiments, each n is 2. In embodiments, each n is 3. In embodiments, each n is 4.

In Formula (III-f) or (III-f′), each R³ can independently be according to any permitted group described herein (e.g., as described for Formula (A′), Formula (A) or Formula (III)).

In embodiments of Formula (III-f) or (III-f′), each n is 3. In embodiments, each n is 2. In embodiments, each n is 4. In embodiments, each n is 5. In embodiments, each n is 6. In embodiments, each n is 7. In embodiments, each n is 8. In embodiments, each n is 9. In embodiments, each n is 10.

Compounds of Formula (IV)

In embodiments, the cationic lipid of Formula (A′) has the following structure:

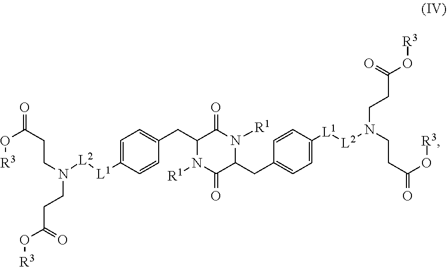

or a pharmaceutically acceptable salt thereof, wherein
each R¹ is independently H or C₁-C₆ aliphatic;
each L¹ is independently an ester, thioester, disulfide, or anhydride group;
each L² is independently C₂-C₁₀ aliphatic;
each R³ is independently C₆-C₃₀ aliphatic.

In some embodiments of Formula (IV), each R³ is independently C₆-C₂₀ aliphatic. In some embodiments of Formula (IV), each R³ is independently C₈-C₂₀ aliphatic.

In Formula (IV), each R¹, L¹, L², and R³ can independently be according to any permitted group recited in any aspect or embodiment described herein (e.g., as described for Formula (A′), (A), or Formula (I)).

In embodiments of Formula (IV), each R¹ is independently H or C₁-C₆ alkyl. In embodiments of Formula (IV), each R¹ is H.

Compounds of Formula (IV-a)

In embodiments, the cationic lipid of Formula (IV) has the following structure:

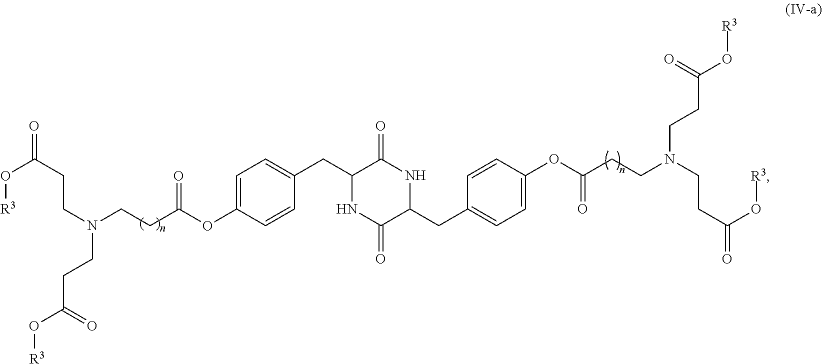

or a pharmaceutically acceptable salt thereof, wherein each n is an integer having a value of 1 to 9.

In some embodiments of Formula (IV-a), each R³ is independently C₆-C₃₀ aliphatic. In some embodiments of Formula (IV-a), each R³ is independently C₆-C₂₀ aliphatic. In some embodiments of Formula (IV-a), each R³ is independently C₈-C₂₀ aliphatic.

In embodiments, the cationic lipid of Formula (IV) or Formula (IV-a) has the following structure:

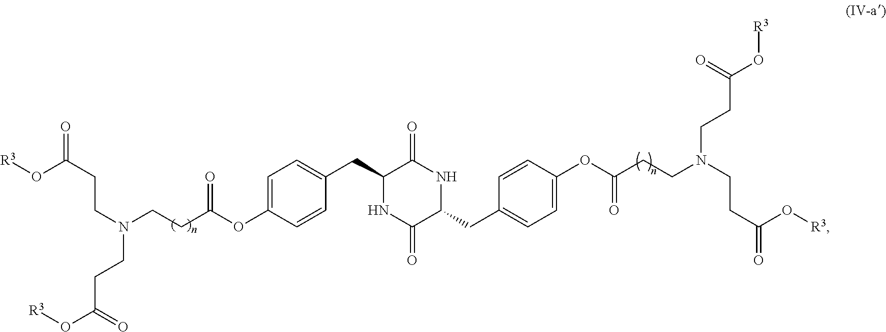

or a pharmaceutically acceptable salt thereof.

In some embodiments of Formula (IV-a′), each R³ is independently C₆-C₃₀ aliphatic. In some embodiments of Formula (IV-a′), each R³ is independently C₆-C₂₀ aliphatic. In some embodiments of Formula (IV-a′), each R³ is independently C₈-C₂₀ aliphatic.

In embodiments, each n is 2.

In Formula (IV-a) or (IV-a′), each R³ can independently be according to any permitted group described herein (e.g., as described for Formula (A′), Formula (A) or Formula (III)).

In embodiments of Formula (IV-a) or (IV-a′), each n is 2. In embodiments, each n is 1. In embodiments, each n is 3. In embodiments, each n is 4. In embodiments, each n is 5. In embodiments, each n is 6. In embodiments, each n is 7. In embodiments, each n is 8. In embodiments, each n is 9.

In embodiments of any formula described herein (e.g., any of Formula (A′), (A), (I), (I-a), (I-al, (I-b), (I-b′), (I-c), (I-c′), (I-c-1), (I-c′-1), (I-c-2), (I-c′-2), (I-d), (I-d′), (I-d-1), (I-d-2), (I-e), (I-e′), (I-e-1), (I-e-2), (I-f), (I-f′), (II), (II-a), (II-al, (III), (III′), (III-a), (III-a′), (III-b), (III-b′), (III-c), (III-c-1), (III-c′-1), (III-c-2), (III-c′-2), (III-c′), (III-d), (III-d′), (III-d-1), (III-d-2), (III-e), (III-e′), (III-e-1), (III-e-2), (III-f), (III-f′), (IV), (IV-a), or (IV-al), the cationic lipid has a structure according to each R³ is unsubstituted C₆-C₂₀ alkyl (e.g., each R³ is C₆H₁₃, C₈H₁₇, C₁₀H₂₁, C₁₂H₂₅, C₁₄H₂₉, C₁₆H₃₃, or C₁₈H₃₇). In embodiments, each R³ is unsubstituted C₈-C₂₀ alkyl. In embodiments, each R³ is C₆H₁₃. In embodiments, each R³ is C₈H₁₇. In embodiments, each R³ is C₁₀H₂₁. In embodiments, each R³ is C₁₂H₂₅. In embodiments, each R³ is C₁₄H₂₉. In embodiments, each R³ is C₁₆H₃₃. In embodiments, each R³ is C₁₈H₃₇.

In embodiments of any formula described herein (e.g., any of Formula (A′), (A), (I), (I-a), (I-a′), (I-b), (I-b′), (I-c), (I-c′), (I-c-1), (I-c′-1), (I-c-2), (I-c′-2), (I-d), (I-d′), (I-d-1), (I-d-2), (I-e), (I-e′), (I-e-1), (I-e-2), (I-f), (I-f′), (II), (II-a), (II-a′), (III), (III′), (III-a), (III-a′), (III-b), (III-b′), (III-c), (III-c-1), (III-c′-1), (III-c-2), (III-c′-2), (III-c′), (III-d), (III-d′), (III-d-1), (III-d-2), (III-e), (III-e′), (III-e-1), (III-e-2), (III-f), (III-f′), (IV), (IV-a), or (IV-al), each R³ is substituted C₆-C₂₀ alkyl. In embodiments, R³ comprises a substituent that is —O—C(O)R′ or —C(O)—OR′, wherein R′ is C₁-C₁₆ alkyl. In embodiments, R³ is C₆-C₁₀ alkyl substituted by —O—C(O)C₇H₁₅ or —C(O)—O—(CH₂)₂CH(C₅H₁₁)₂. In embodiments, R³ is C₆ alkyl substituted by —O—C(O)R′ or —C(O)—OR′, wherein R′ is unsubstituted C₅-C₁₆ alkyl that is linear or branched such as —O—C(O)C₇H₁₅ or —C(O)—O—(CH₂)₂CH(C₅H₁₁)₂). In embodiments, R³ is C₇ alkyl substituted by —O—C(O)R′ or —C(O)—OR′, wherein R′ is unsubstituted C₅-C₁₆ alkyl that is linear or branched such as —O—C(O)C₇H₁₅ or —C(O)—O—(CH₂)₂CH(C₅H₁₁)₂). In embodiments, R³ is C₈ alkyl substituted by —O—C(O)R′ or —C(O)—OR′, wherein R′ is unsubstituted C₅-C₁₆ alkyl that is linear or branched such as —O—C(O)C₇H₁₅ or —C(O)—O—(CH₂)₂CH(C₅H₁₁)₂). In embodiments, R³ is C₉ alkyl substituted by —O—C(O)R′ or —C(O)—OR′, wherein R′ is unsubstituted C₅-C₁₆ alkyl that is linear or branched such as —O—C(O)C₇H₁₅ or —C(O)—O—(CH₂)₂CH(C₅H₁₁)₂). In embodiments, R³ is C₁₀ alkyl substituted by —O—C(O)R′ or —C(O)—OR′, wherein R′ is unsubstituted C₅-C₁₆ alkyl that is linear or branched such as —O—C(O)C₇H₁₅ or —C(O)—O—(CH₂)₂CH(C₅H₁₁)₂). In embodiments, each R³ is —(CH₂)₉—O—C(O)C₇H₁₅ or —(CH₂)₈C(O)—O—(CH₂)₂CH(C₅H₁₁)₂.

In embodiments of any formula described herein (e.g., any of Formula (A′), (A), (I), (I-a), (I-a′), (I-b), (I-b′), (I-c), (I-c′), (I-c-1), (I-c′-1), (I-c-2), (I-c′-2), (I-d), (I-d′), (I-d-1), (I-d-2), (I-e), (I-e′), (I-e-1), (I-e-2), (I-f), (I-f′), (II), (II-a), (II-a′), (III), (III′), (III-a), (III-a′), (III-b), (III-b′), (III-c), (III-c-1), (III-c′-1), (III-c-2), (III-c′-2), (III-c′), (III-d), (III-d′), (III-d-1), (III-d-2), (III-e), (III-e′), (III-e-1), (III-e-2), (III-f), (III-f′), (IV), (IV-a), or (IV-a′), each R³ is unsubstituted C₆-C₂₀ alkenyl (e.g., each R³ is C₁₆H₃₁ or C₁₆H₂₉). In embodiments, each R³ is unsubstituted C₈-C₂₀ alkenyl. In embodiments, each R³ is unsubstituted C₁₀-C₂₀ alkenyl. In embodiments, each R³ is unsubstituted monoalkenyl, unsubstituted dienyl, or unsubstituted trienyl. In embodiments, each R³ is unsubstituted C₆-C₂₀ monoalkenyl. In embodiments, each R³ is unsubstituted C₆-C₂₀ unsubstituted dienyl. In embodiments, each R³ is unsubstituted C₆-C₂₀ unsubstituted trienyl. In embodiments, each R³ is —(CH₂)ₒR′, wherein o is 6, 7, 8, 9, or 10, and R′ is

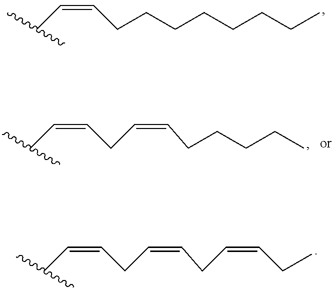 [SMILES: */C=C\CCCCCCCC ; */C=C\C/C=C\CCCCC ; */C=C\C/C=C\C/C=C\CC]

In embodiments, o is 6. In embodiments, o is 7. In embodiments, o is 8. In embodiments, o is 9. In embodiments, o is 10. In embodiments, R′ is

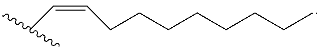 [SMILES: */C=C\CCCCCCCC]

In embodiments, R′ is

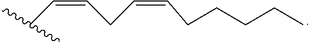 [SMILES: */C=C\C/C=C\CCCCC]

In embodiments, R′ is

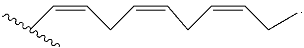 [SMILES: */C=C\C/C=C\C/C=C\CC]

In embodiments, each R³ is C₁₆H₃₁. In embodiments, each R³ is C₁₆H₂₉.

In embodiments of any formula described herein (e.g., any of Formula (A′), (A), (I), (I-a), (I-a′), (I-b), (I-b′), (I-c), (I-c′), (I-c-1), (I-c′-1), (I-c-2), (I-c′-2), (I-d), (I-d′), (I-d-1), (I-d-2), (I-e), (I-e′), (I-e-1), (I-e-2), (I-f), (I-f′), (II), (II-a), (II-a′), (III), (III′), (III-a), (III-a′), (III-b), (III-b′), (III-c), (III-c-1), (III-c′-1), (III-c-2), (III-C′-2), (III-c′), (III-d), (III-d′), (III-d-1), (III-d-2), (III-e), (III-e′), (III-e-1), (III-e-2), (III-f), (III-f), (IV), (IV-a), or (IV-a′)), each R³ is unsubstituted C₆-C₂₀ alkynyl. In embodiments, each R³ is unsubstituted C₈-C₂₀ alkynyl. In embodiments, each R³ is unsubstituted C₁₀-C₂₀ alkynyl.

In embodiments of any formula described herein (e.g., any of Formula (A′), (A), (I), (I-a), (I-a′), (I-b), (I-b′), (I-c), (I-c′), (I-c-1), (I-C′-1), (I-c-2), (I-C′-2), (I-d), (I-d′), (I-d-1), (I-d-2), (I-e), (I-e′), (I-e-1), (I-e-2), (I-f), (I-f′), (II), (II-a), (II-a′), (III), (III′), (III-a), (III-a′), (III-b), (III-b′), (III-c), (III-c-1), (III-c′-1), (III-c-2), (III-C′-2), (III-c′), (III-d), (III-d′), (III-d-1), (III-d-2), (III-e), (III-e′), (III-e-1), (III-e-2), (III-f), (III-f), (IV), (IV-a), or (IV-a′)), each R³ is unsubstituted C₆-C₃₀ alkynyl. In embodiments, each R³ is unsubstituted C₈-C₃₀ alkynyl. In embodiments, each R³ is unsubstituted C₁₀-C₃₀ alkynyl.

### Exemplary Compounds of the Invention

Exemplary compounds include any of those described in Tables A-P.

In these tables, substructure a=—(CH₂)₉—O—C(O)—C₇H₁₅, and substructureb=—(CH₂)₈—C(O)—O—CH₂CH₂CH(C₅H₁₁)₂.

TABLE A
cDD Thioesters

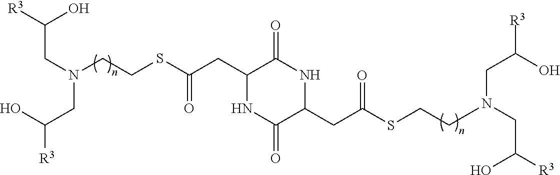

| No. | n | R³ | Structure |
|---|---|---|---|
| 1 | 1 | C₈H₁₇ | 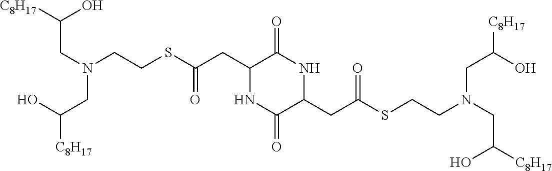 |
| 2 | 2 | C₈H₁₇ | 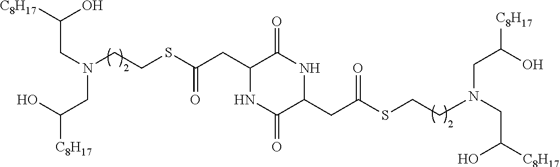 |
| 3 | 3 | C₈H₁₇ | 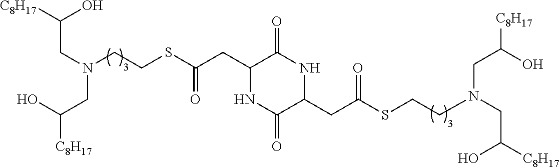 |
| 4 | 1 | C₁₀H₂₁ | 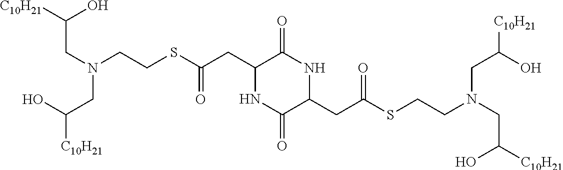 |
| 5 | 2 | C₁₀H₂₁ | 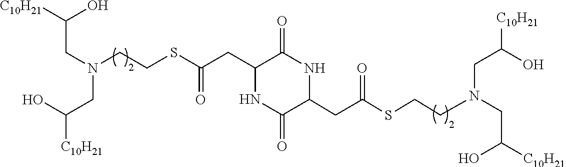 |
| 6 | 3 | C₁₀H₂₁ | 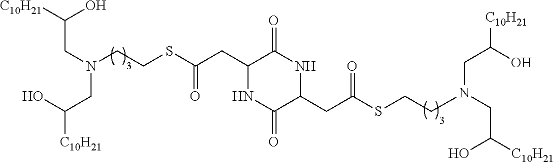 |
| 7 | 1 | C₁₂H₂₅ | 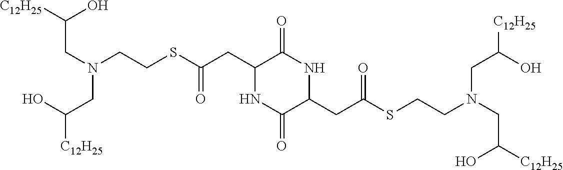 |
| 8 | 2 | C₁₂H₂₅ | 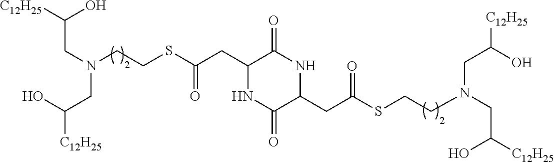 |
| 9 | 3 | C₁₂H₂₅ | 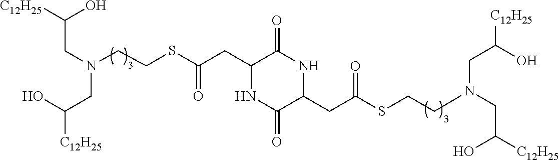 |
| 10 | 1 | C₁₄H₂₉ | 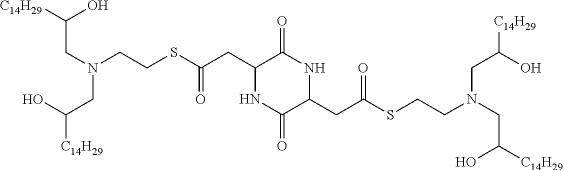 |
| 11 | 2 | C₁₄H₂₉ | 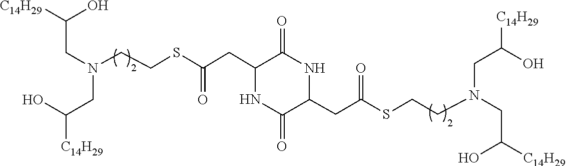 |
| 12 | 3 | C₁₄H₂₉ | 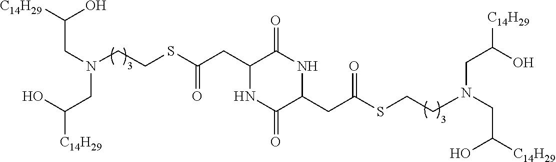 |
| 13 | 1 | C₁₆H₃₃ | 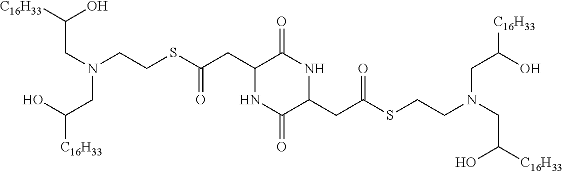 |
| 14 | 2 | C₁₆H₃₃ | 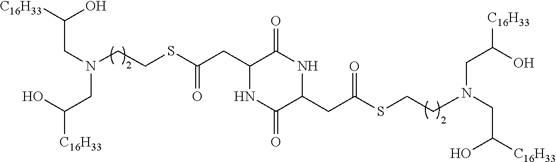 |
| 15 | 3 | C₁₆H₃₃ | 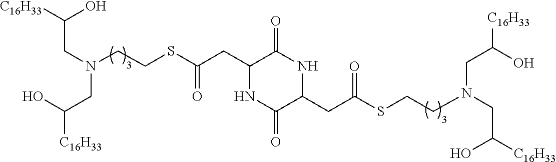 |
| 16 | 1 | C₁₆H₃₁ | 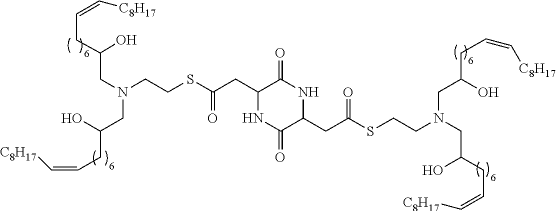 |
| 17 | 2 | C₁₆H₃₁ | 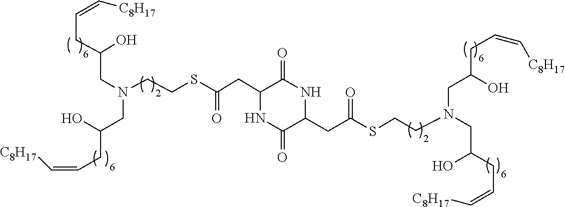 |
| 18 | 3 | C₁₆H₃₁ | 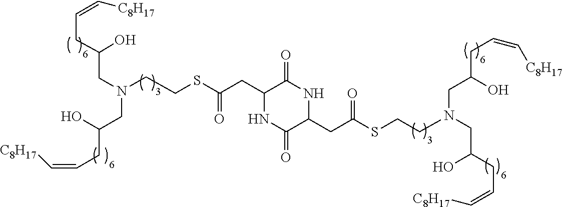 |
| 19 | 1 | C₁₆H₂₉ | 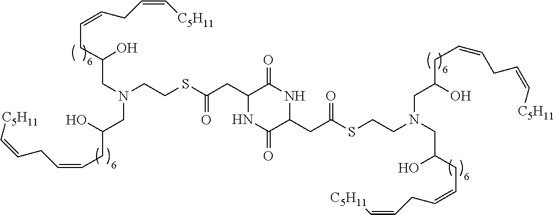 |
| 20 | 2 | C₁₆H₂₉ | 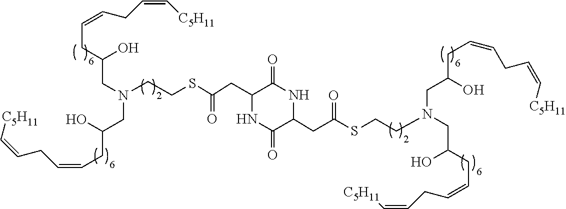 |
| 21 | 3 | C₁₆H₂₉ | 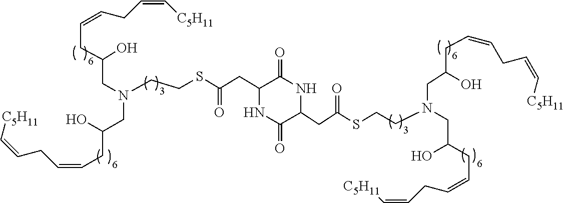 |
| 22 | 1 | C₆H₁₃ | 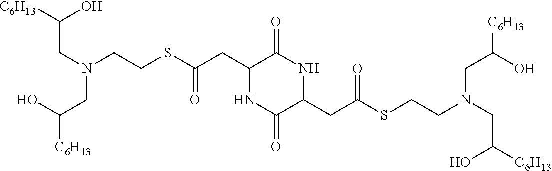 |
| 23 | 2 | C₆H₁₃ | 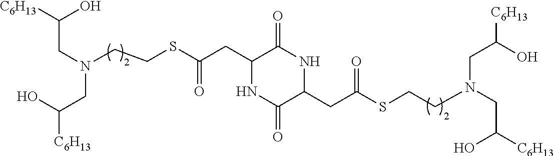 |
| 24 | 3 | C₆H₁₃ | 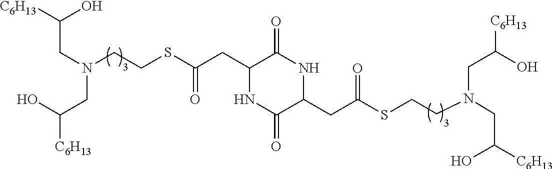 |
| 25 | 1 | substructure a | 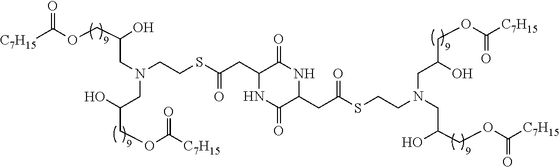 |
| 26 | 2 | substructure a | 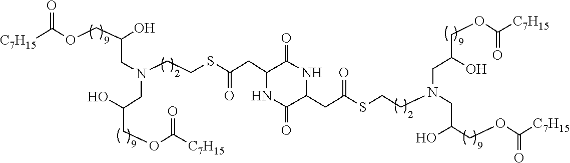 |
| 27 | 3 | substructure a | 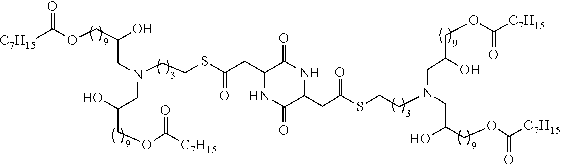 |
| 28 | 1 | substructure b | 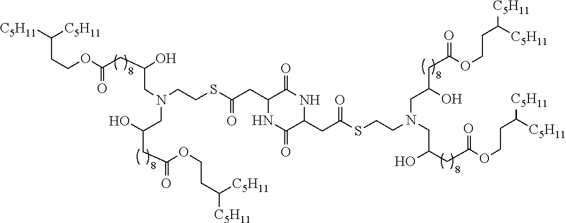 |
| 29 | 2 | substructure b | 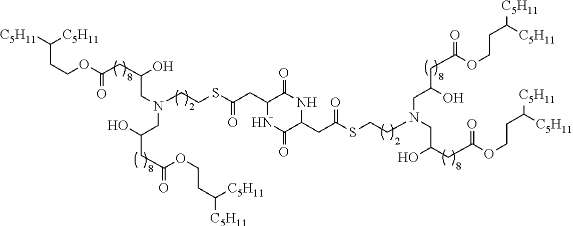 |
| 30 | 3 | substructure b | 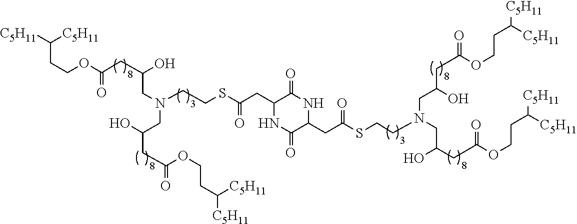 |

In embodiments, a cationic lipid is Compound 1. In embodiments, a cationic lipid is Compound 2. In embodiments, a cationic lipid is Compound 3. In embodiments, a cationic lipid is Compound 4. In embodiments, a cationic lipid is Compound 5. In embodiments, a cationic lipid is Compound 6. In embodiments, a cationic lipid is Compound 7. In embodiments, a cationic lipid is Compound 8. In embodiments, a cationic lipid is Compound 9. In embodiments, a cationic lipid is Compound 10. In embodiments, a cationic lipid is Compound 11. In embodiments, a cationic lipid is Compound 12. In embodiments, a cationic lipid is Compound 13. In embodiments, a cationic lipid is Compound 14. In embodiments, a cationic lipid is Compound 15. In embodiments, a cationic lipid is Compound 16. In embodiments, a cationic lipid is Compound 17. In embodiments, a cationic lipid is Compound 18. In embodiments, a cationic lipid is Compound 19. In embodiments, a cationic lipid is Compound 20. In embodiments, a cationic lipid is Compound 21. In embodiments, a cationic lipid is Compound 22. In embodiments, a cationic lipid is Compound 23. In embodiments, a cationic lipid is Compound 24. In embodiments, a cationic lipid is Compound 25. In embodiments, a cationic lipid is Compound 26. In embodiments, a cationic lipid is Compound 27. In embodiments, a cationic lipid is Compound 28. In embodiments, a cationic lipid is Compound 29. In embodiments, a cationic lipid is Compound 30.

TABLE B
cDD Esters

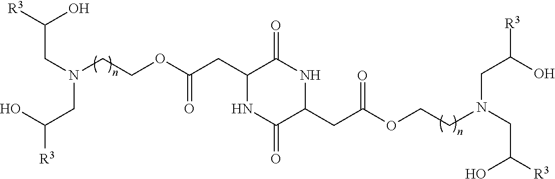

| No. | n | R³ | Structure |
|---|---|---|---|
| 31 | 1 | C₈H₁₇ | 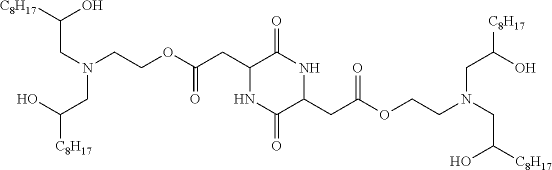 |
| 32 | 2 | C₈H₁₇ | 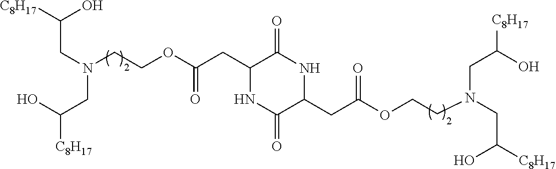 |
| 33 | 3 | C₈H₁₇ | 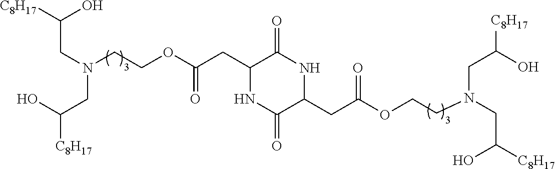 |
| 34 | 1 | C₁₀H₂₁ | 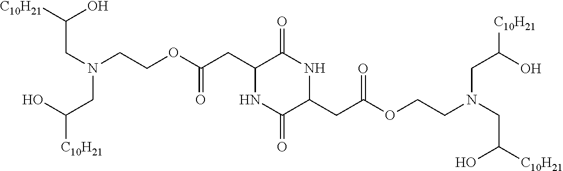 |
| 35 | 2 | C₁₀H₂₁ | 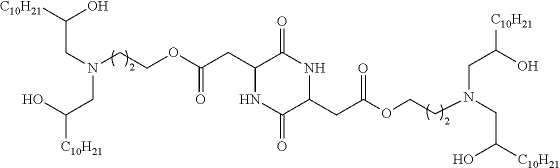 |
| 36 | 3 | C₁₀H₂₁ | 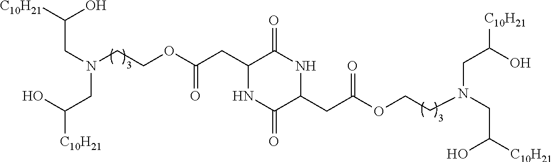 |
| 37 | 1 | C₁₂H₂₅ | 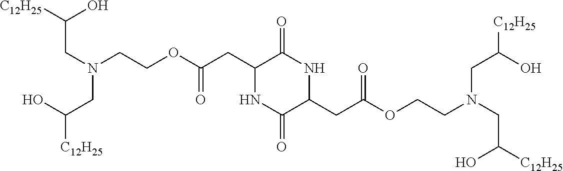 |
| 38 | 2 | C₁₂H₂₅ | 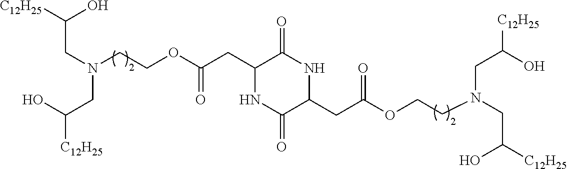 |
| 39 | 3 | C₁₂H₂₅ | 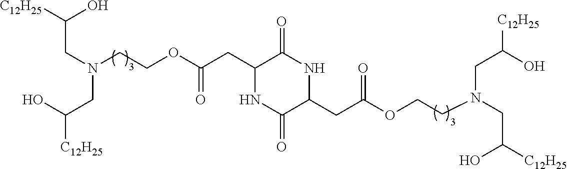 |
| 40 | 1 | C₁₄H₂₉ | 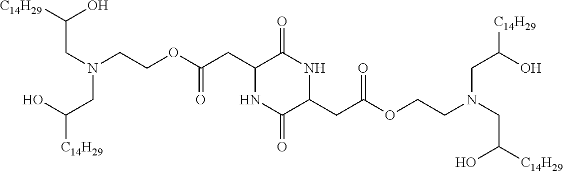 |
| 41 | 2 | C₁₄H₂₉ | 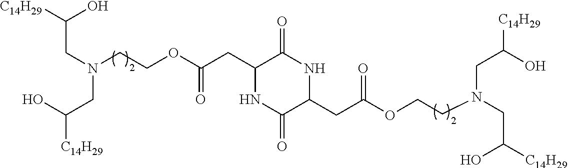 |
| 42 | 3 | C₁₄H₂₉ | 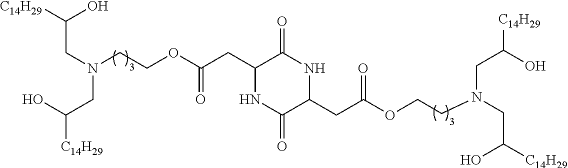 |
| 43 | 1 | C₁₆H₃₃ | 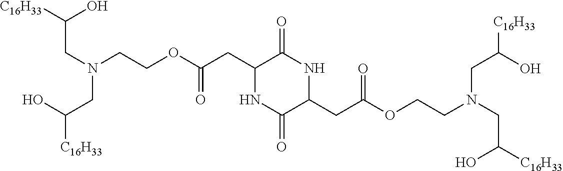 |
| 44 | 2 | C₁₆H₃₃ | 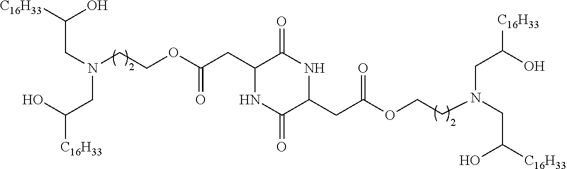 |
| 45 | 3 | C₁₆H₃₃ | 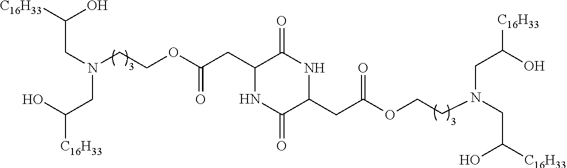 |
| 46 | 1 | C₁₆H₃₁ | 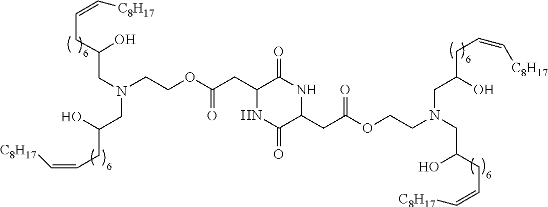 |
| 47 | 2 | C₁₆H₃₁ | 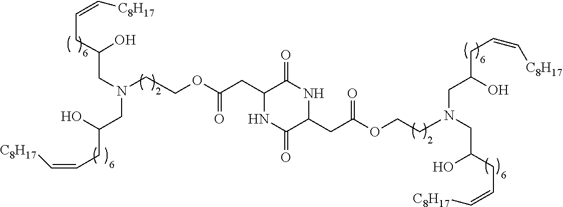 |
| 48 | 3 | C₁₆H₃₁ | 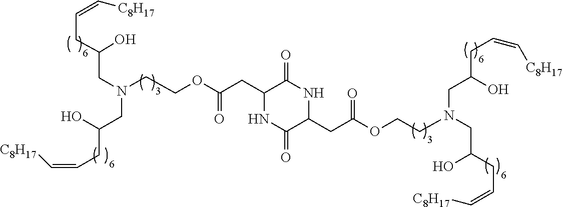 |
| 49 | 1 | C₁₆H₂₉ | 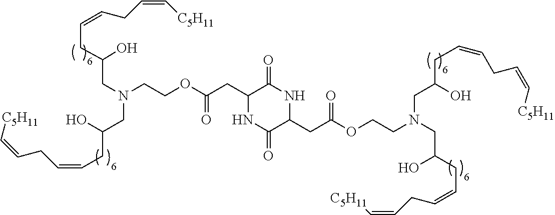 |
| 50 | 2 | C₁₆H₂₉ | 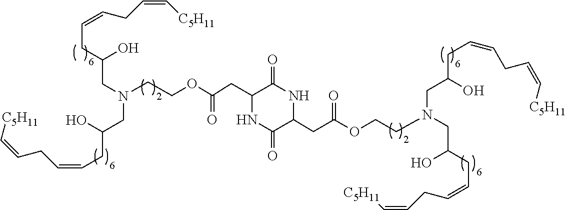 |
| 51 | 3 | C₁₆H₂₉ | 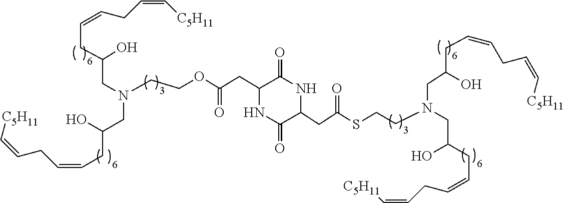 |
| 52 | 1 | C₆H₁₃ | 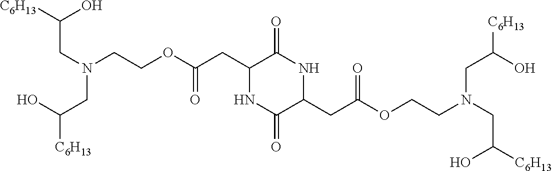 |
| 53 | 2 | C₆H₁₃ | 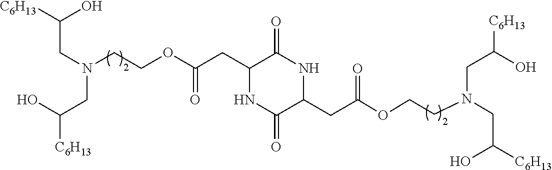 |
| 54 | 3 | C₆H₁₃ | 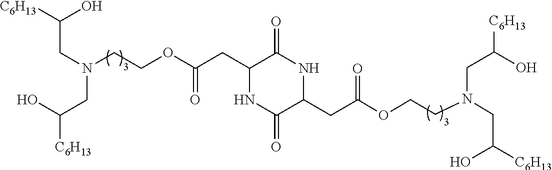 |
| 55 | 1 | substructure a | 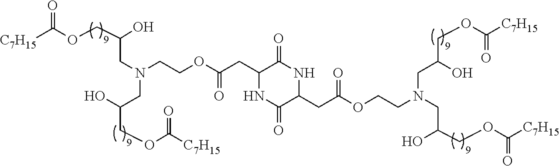 |
| 56 | 2 | substructure a | 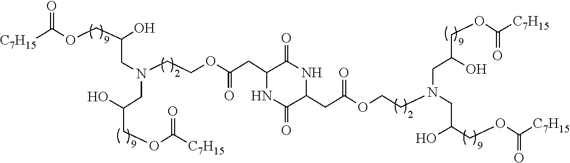 |
| 57 | 3 | substructure a | 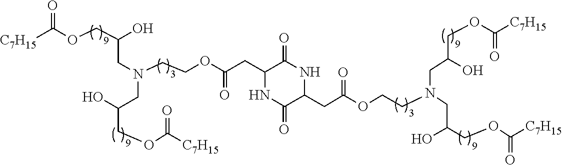 |
| 58 | 1 | substructure b | 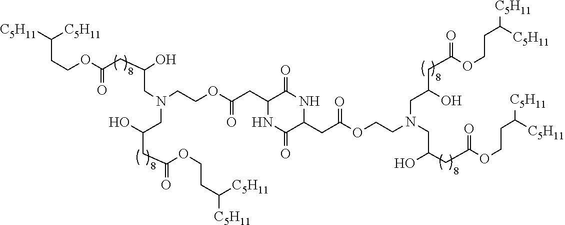 |
| 59 | 2 | substructure b | 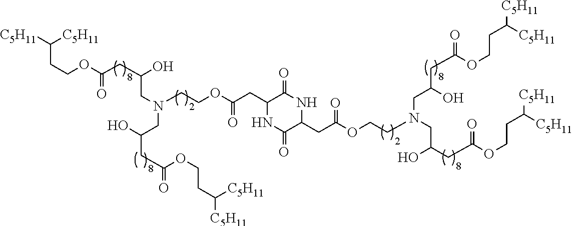 |
| 60 | 3 | substructure b | 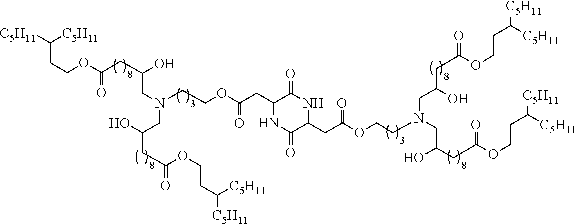 |

In embodiments, a cationic lipid is Compound 31. In embodiments, a cationic lipid is Compound 32. In embodiments, a cationic lipid is Compound 33. In embodiments, a cationic lipid is Compound 34. In embodiments, a cationic lipid is Compound 35. In embodiments, a cationic lipid is Compound 36. In embodiments, a cationic lipid is Compound 37. In embodiments, a cationic lipid is Compound 38. In embodiments, a cationic lipid is Compound 39. In embodiments, a cationic lipid is Compound 40. In embodiments, a cationic lipid is Compound 41. In embodiments, a cationic lipid is Compound 42. In embodiments, a cationic lipid is Compound 43. In embodiments, a cationic lipid is Compound 44. In embodiments, a cationic lipid is Compound 45. In embodiments, a cationic lipid is Compound 46. In embodiments, a cationic lipid is Compound 47. In embodiments, a cationic lipid is Compound 48. In embodiments, a cationic lipid is Compound 49. In embodiments, a cationic lipid is Compound 50. In embodiments, a cationic lipid is Compound 51. In embodiments, a cationic lipid is Compound 52. In embodiments, a cationic lipid is Compound 53. In embodiments, a cationic lipid is Compound 54. In embodiments, a cationic lipid is Compound 55. In embodiments, a cationic lipid is Compound 56. In embodiments, a cationic lipid is Compound 57. In embodiments, a cationic lipid is Compound 58. In embodiments, a cationic lipid is Compound 59. In embodiments, a cationic lipid is Compound 60.

TABLE C
cEE Thioesters

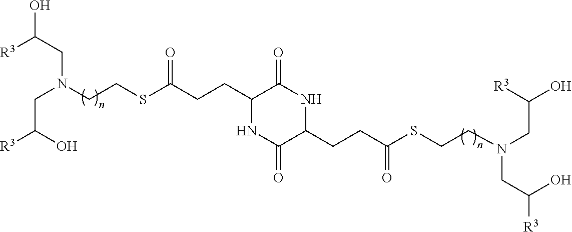

| No. | n | R³ | Structure |
|---|---|---|---|
| 61 | 1 | C₈H₁₇ | 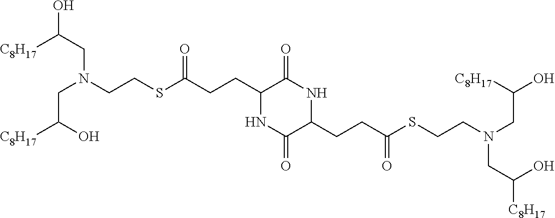 |
| 62 | 2 | C₈H₁₇ | 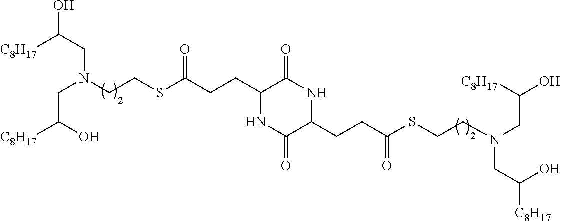 |
| 63 | 3 | C₈H₁₇ | 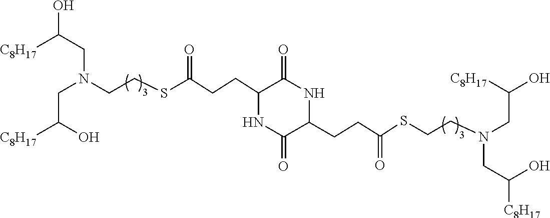 |
| 64 | 1 | C₁₀H₂₁ | 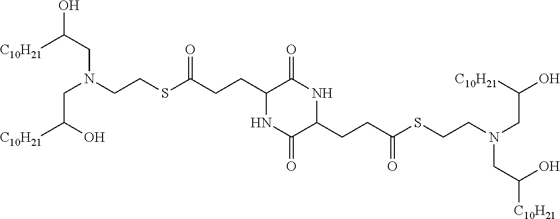 |
| 65 | 2 | C₁₀H₂₁ | 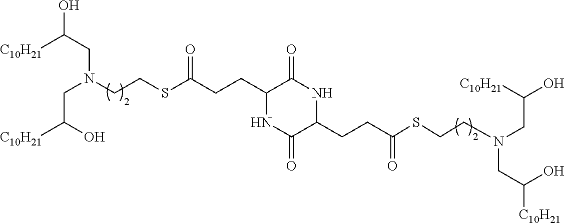 |
| 66 | 3 | C₁₀H₂₁ | 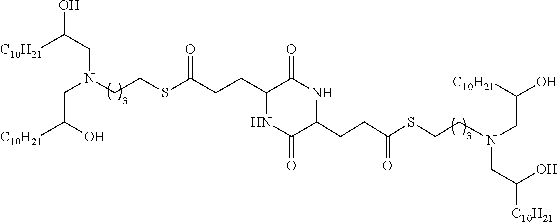 |
| 67 | 1 | C₁₂H₂₅ | 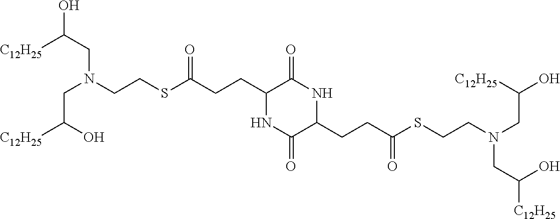 |
| 68 | 2 | C₁₂H₂₅ | 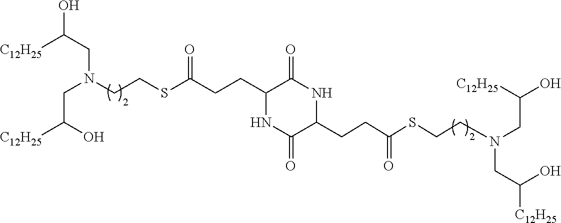 |
| 69 | 3 | C₁₂H₂₅ | 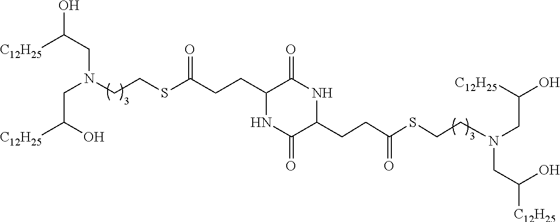 |
| 70 | 1 | C₁₄H₂₉ | 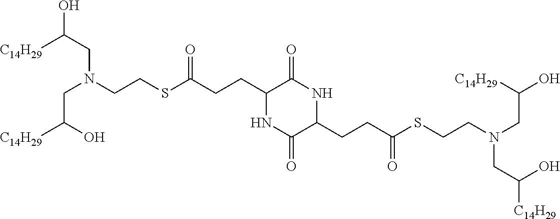 |
| 71 | 2 | C₁₄H₂₉ | 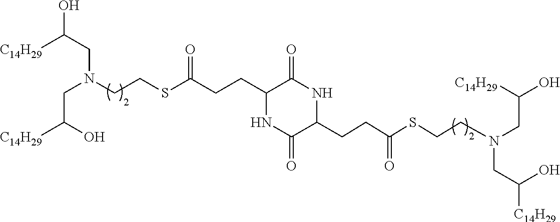 |
| 72 | 3 | C₁₄H₂₉ | 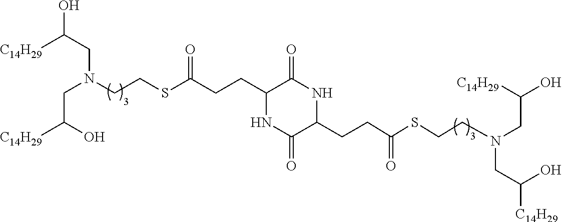 |
| 73 | 1 | C₁₆H₃₃ | 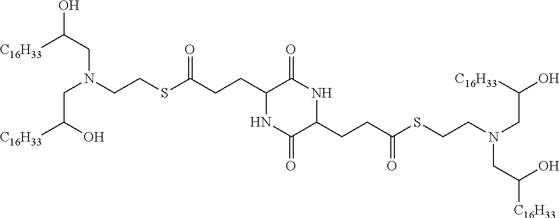 |
| 74 | 2 | C₁₆H₃₃ | 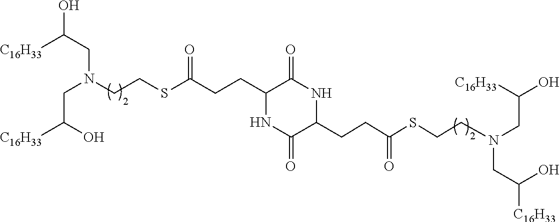 |
| 75 | 3 | C₁₆H₃₃ | 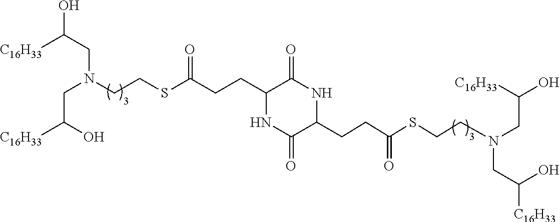 |
| 76 | 1 | C₁₆H₃₁ | 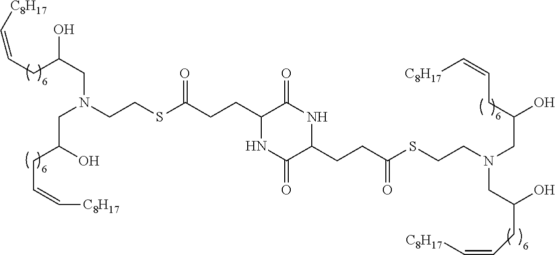 |
| 77 | 2 | C₁₆H₃₁ | 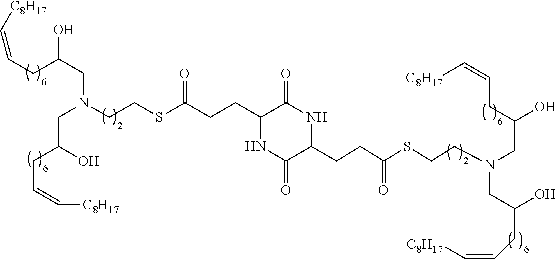 |
| 78 | 3 | C₁₆H₃₁ | 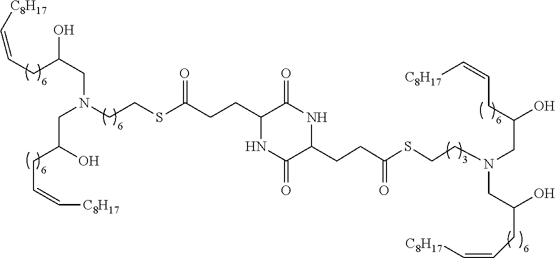 |
| 79 | 1 | C₁₆H₂₉ | 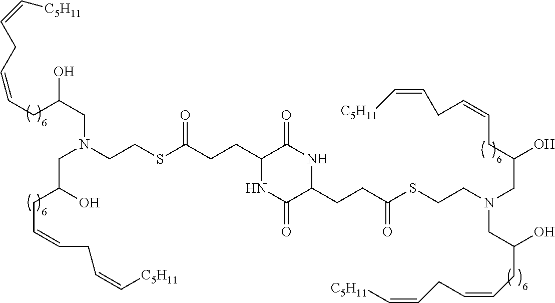 |
| 80 | 2 | C₁₆H₂₉ | 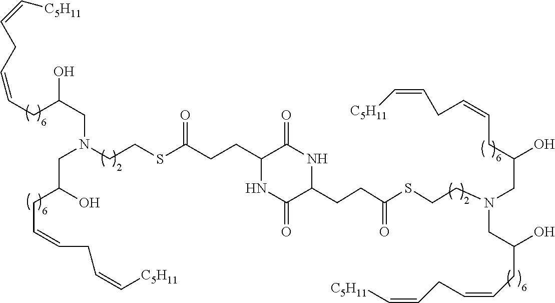 |
| 81 | 3 | C₁₆H₂₉ | 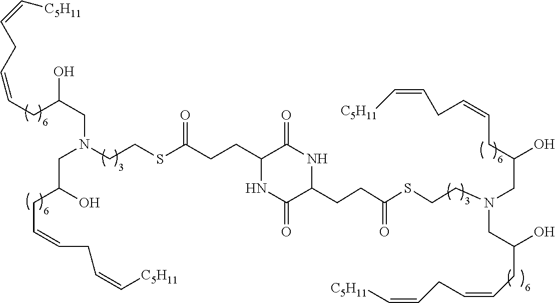 |
| 82 | 1 | C₆H₁₃ | 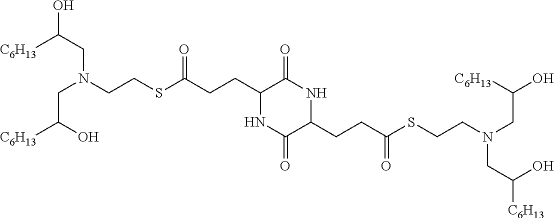 |
| 83 | 2 | C₆H₁₃ | 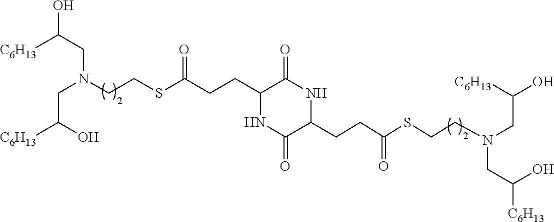 |
| 84 | 3 | C₆H₁₃ | 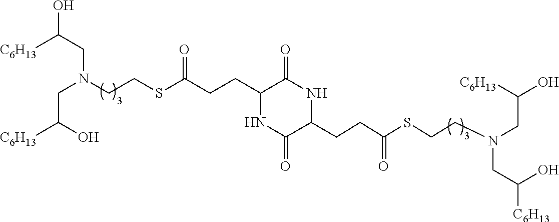 |
| 85 | 1 | substructure a | 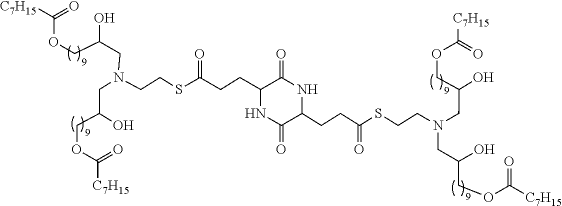 |
| 86 | 2 | substructure a | 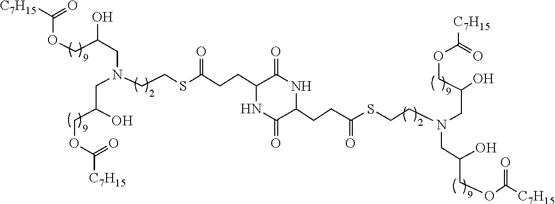 |
| 87 | 3 | substructure a | 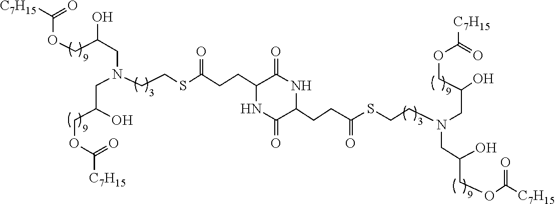 |
| 88 | 1 | substructure b | 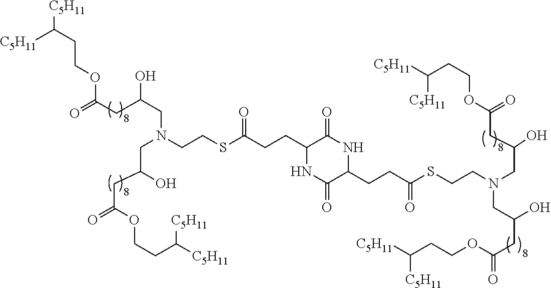 |
| 89 | 2 | substructure b | 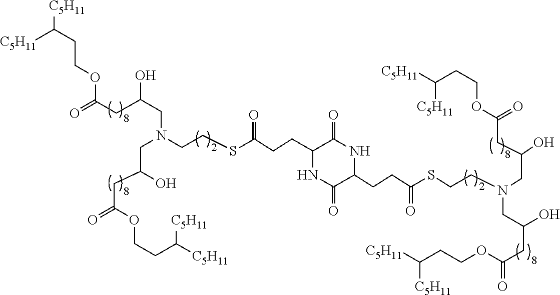 |
| 90 | 3 | substructure b | 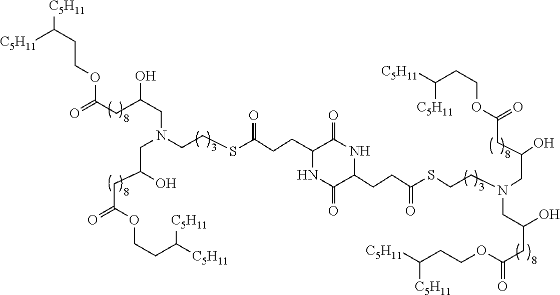 |

In embodiments, a cationic lipid is Compound 61. In embodiments, a cationic lipid is Compound 62. In embodiments, a cationic lipid is Compound 63. In embodiments, a cationic lipid is Compound 64. In embodiments, a cationic lipid is Compound 65. In embodiments, a cationic lipid is Compound 66. In embodiments, a cationic lipid is Compound 67. In embodiments, a cationic lipid is Compound 68. In embodiments, a cationic lipid is Compound 69. In embodiments, a cationic lipid is Compound 70. In embodiments, a cationic lipid is Compound 71. In embodiments, a cationic lipid is Compound 72. In embodiments, a cationic lipid is Compound 73. In embodiments, a cationic lipid is Compound 74. In embodiments, a cationic lipid is Compound 75. In embodiments, a cationic lipid is Compound 76. In embodiments, a cationic lipid is Compound 77. In embodiments, a cationic lipid is Compound 78. In embodiments, a cationic lipid is Compound 79. In embodiments, a cationic lipid is Compound 80. In embodiments, a cationic lipid is Compound 81. In embodiments, a cationic lipid is Compound 82. In embodiments, a cationic lipid is Compound 83. In embodiments, a cationic lipid is Compound 84. In embodiments, a cationic lipid is Compound 85. In embodiments, a cationic lipid is Compound 86. In embodiments, a cationic lipid is Compound 87. In embodiments, a cationic lipid is Compound 88. In embodiments, a cationic lipid is Compound 89. In embodiments, a cationic lipid is Compound 90.

TABLE D
cEE Esters

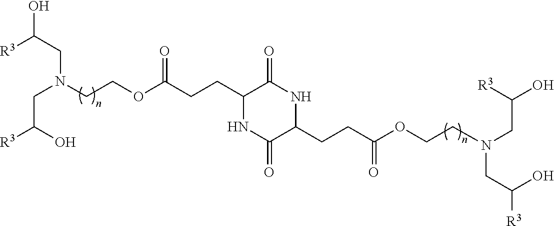

| No. | n | R³ | Structure |
|---|---|---|---|
| 91 | 1 | C₈H₁₇ | 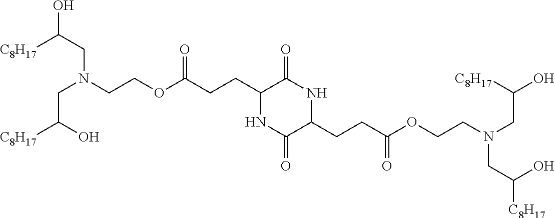 |
| 92 | 2 | C₈H₁₇ | 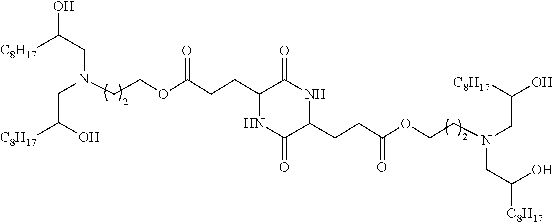 |
| 93 | 3 | C₈H₁₇ | 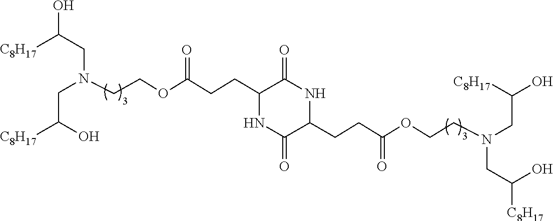 |
| 94 | 1 | C₁₀H₂₁ | 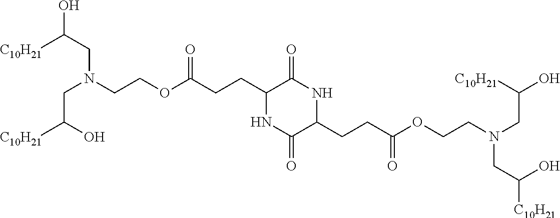 |
| 95 | 2 | C₁₀H₂₁ | 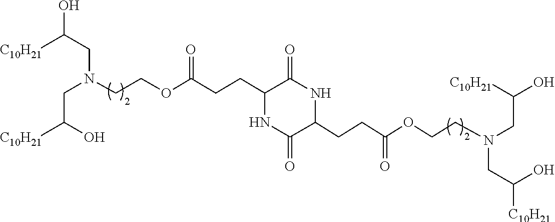 |
| 96 | 3 | C₁₀H₂₁ | 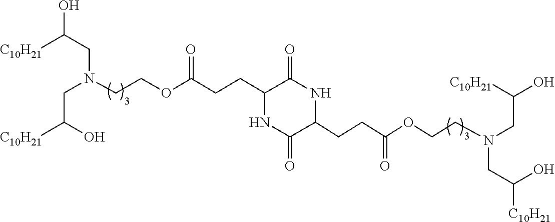 |
| 97 | 1 | C₁₂H₂₅ | 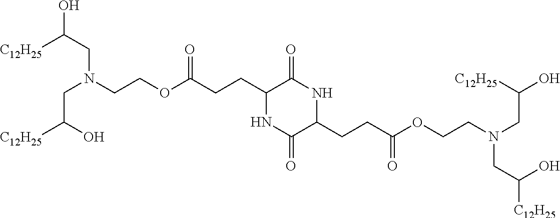 |
| 98 | 2 | C₁₂H₂₅ | 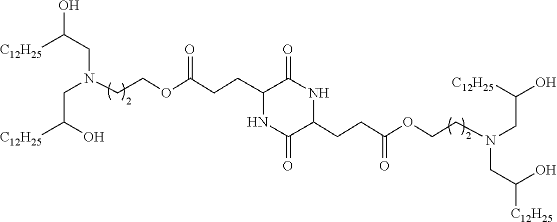 |
| 99 | 3 | C₁₂H₂₅ | 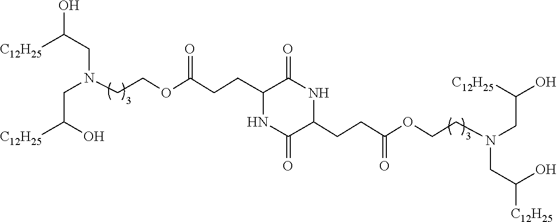 |
| 100 | 1 | C₁₄H₂₉ | 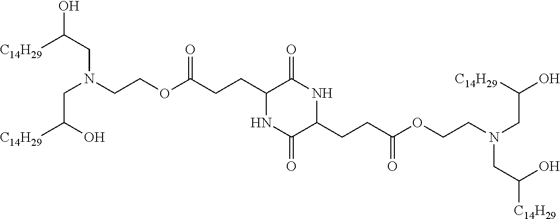 |
| 101 | 2 | C₁₄H₂₉ | 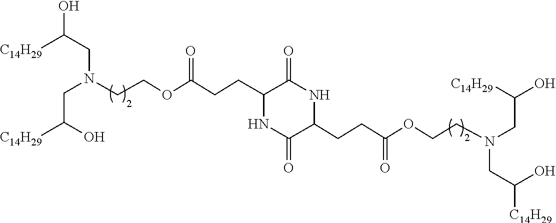 |
| 102 | 3 | C₁₄H₂₉ | 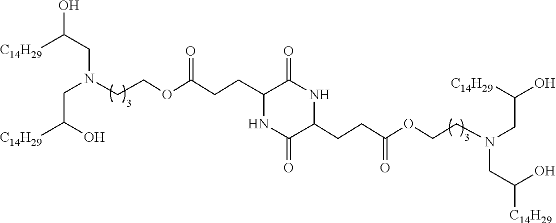 |
| 103 | 1 | C₁₆H₃₃ | 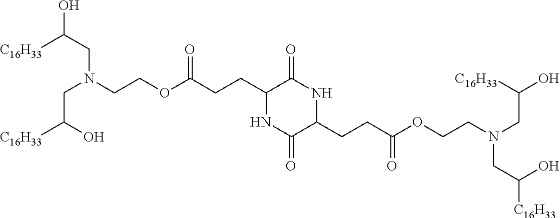 |
| 104 | 2 | C₁₆H₃₃ | 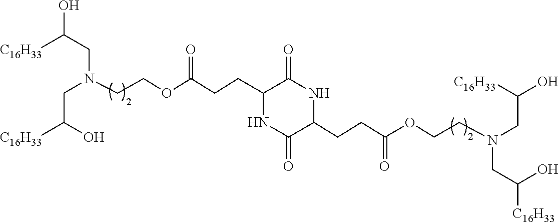 |
| 105 | 3 | C₁₆H₃₃ | 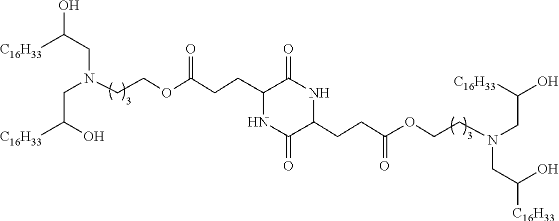 |
| 106 | 1 | C₁₆H₃₁ | 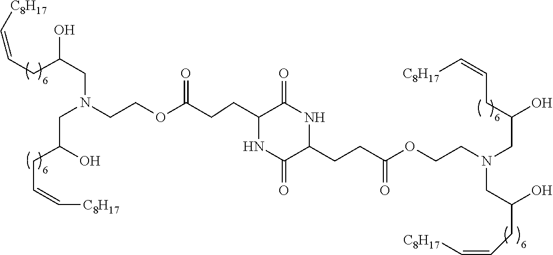 |
| 107 | 2 | C₁₆H₃₁ | 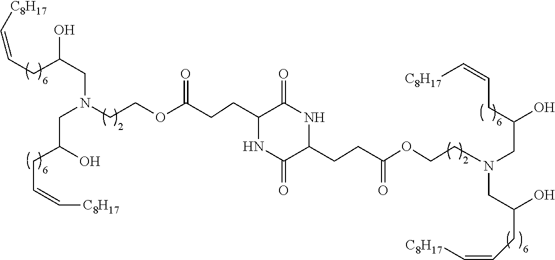 |
| 108 | 3 | C₁₆H₃₁ | 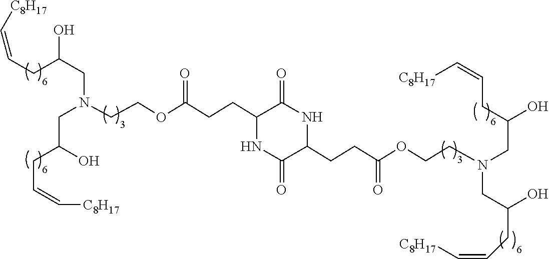 |
| 109 | 1 | C₁₆H₂₉ | 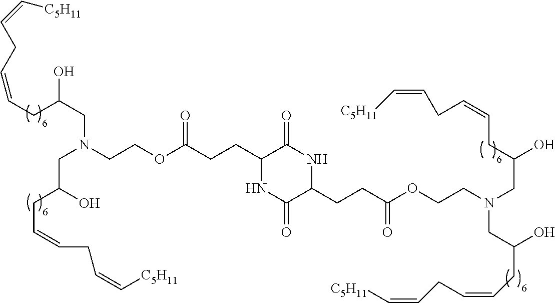 |
| 110 | 2 | C₁₆H₂₉ | 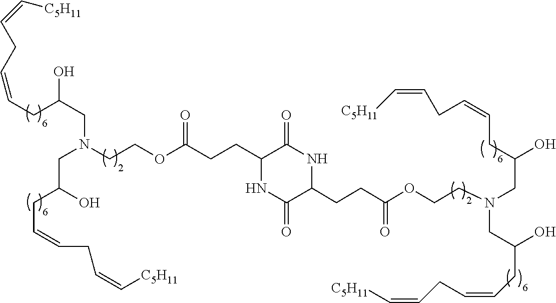 |
| 111 | 3 | C₁₆H₂₉ | 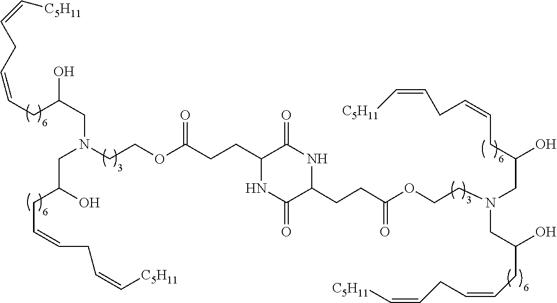 |
| 112 | 1 | C₆H₁₃ | 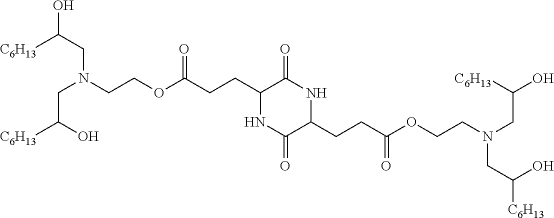 |
| 113 | 2 | C₆H₁₃ | 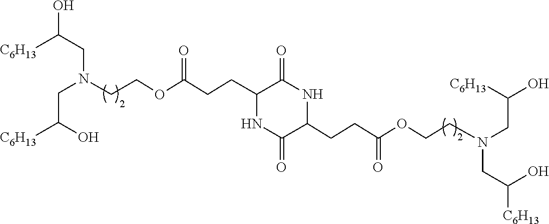 |
| 114 | 3 | C₆H₁₃ | 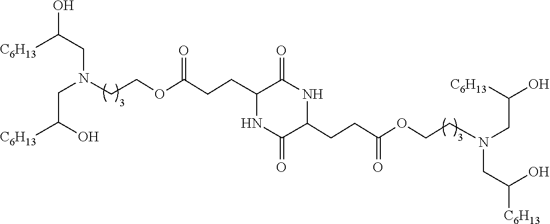 |
| 115 | 1 | substructure a | 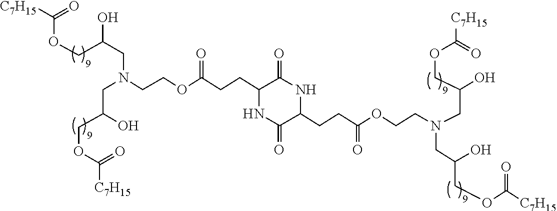 |
| 116 | 2 | substructure a | 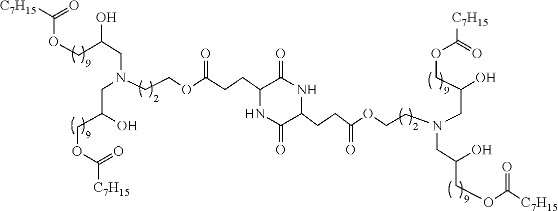 |
| 117 | 3 | substructure a | 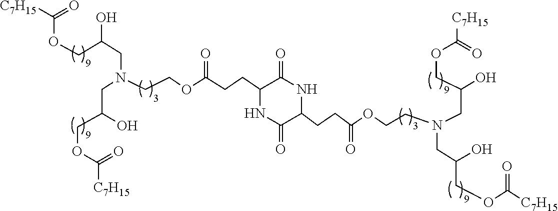 |
| 118 | 1 | substructure b | 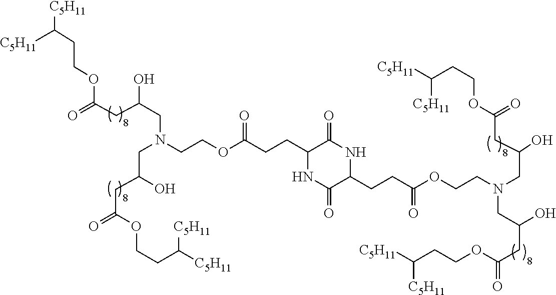 |
| 119 | 2 | substructure b | 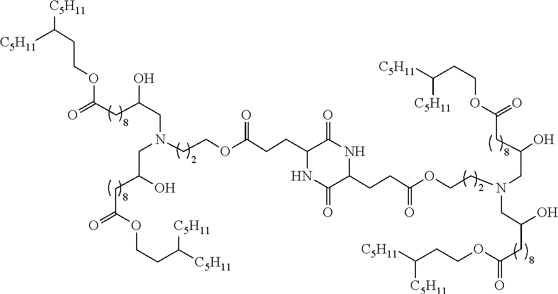 |
| 120 | 3 | substructure b | 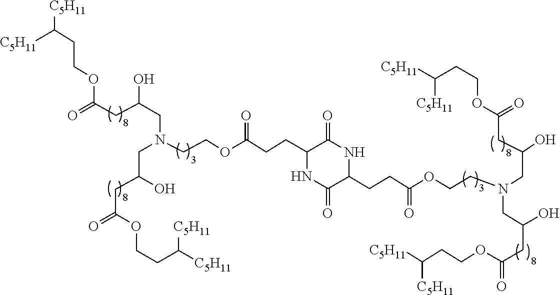 |

In embodiments, a cationic lipid is Compound 91. In embodiments, a cationic lipid is Compound 92. In embodiments, a cationic lipid is Compound 93. In embodiments, a cationic lipid is Compound 94. In embodiments, a cationic lipid is Compound 95. In embodiments, a cationic lipid is Compound 96. In embodiments, a cationic lipid is Compound 97. In embodiments, a cationic lipid is Compound 98. In embodiments, a cationic lipid is Compound 99. In embodiments, a cationic lipid is Compound 100. In embodiments, a cationic lipid is Compound 101. In embodiments, a cationic lipid is Compound 102. In embodiments, a cationic lipid is Compound 103. In embodiments, a cationic lipid is Compound 104. In embodiments, a cationic lipid is Compound 105. In embodiments, a cationic lipid is Compound 106. In embodiments, a cationic lipid is Compound 107. In embodiments, a cationic lipid is Compound 108. In embodiments, a cationic lipid is Compound 109. In embodiments, a cationic lipid is Compound 110. In embodiments, a cationic lipid is Compound 111. In embodiments, a cationic lipid is Compound 112. In embodiments, a cationic lipid is Compound 113. In embodiments, a cationic lipid is Compound 114. In embodiments, a cationic lipid is Compound 115. In embodiments, a cationic lipid is Compound 116. In embodiments, a cationic lipid is Compound 117. In embodiments, a cationic lipid is Compound 118. In embodiments, a cationic lipid is Compound 119. In embodiments, a cationic lipid is Compound 120.

TABLE E
Homoserine (cHse) Lipids

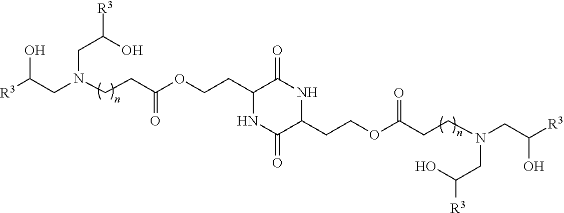

| No. | n | R³ | Structure |
|---|---|---|---|
| 121 | 1 | C₈H₁₇ | 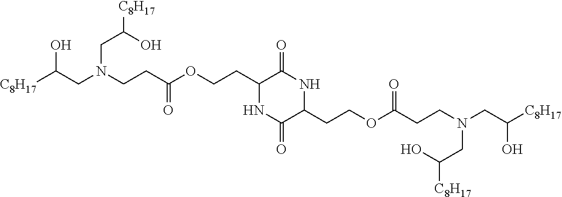 |
| 122 | 2 | C₈H₁₇ | 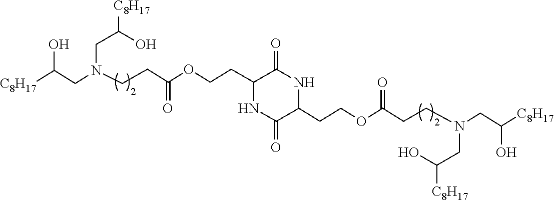 |
| 123 | 3 | C₈H₁₇ | 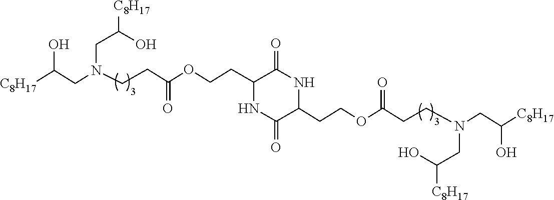 |
| 124 | 1 | C₁₀H₂₁ | 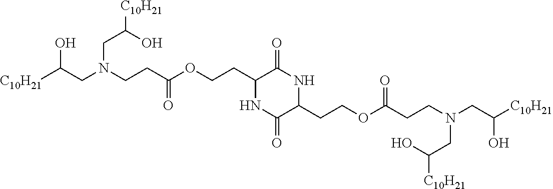 |
| 125 | 2 | C₁₀H₂₁ | 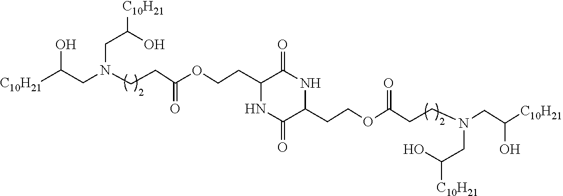 |
| 126 | 3 | C₁₀H₂₁ | 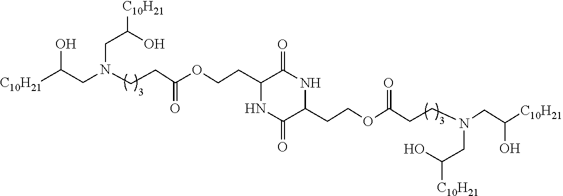 |
| 127 | 1 | C₁₂H₂₅ | 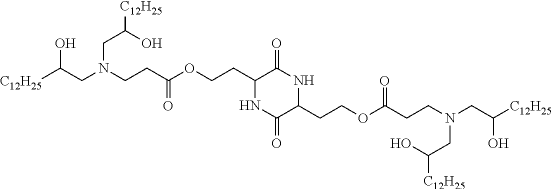 |
| 128 | 2 | C₁₂H₂₅ | 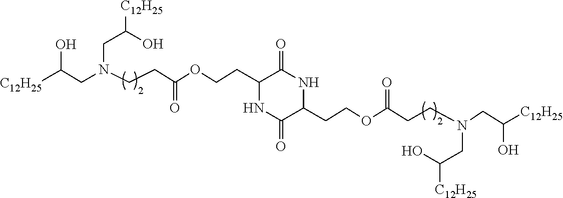 |
| 129 | 3 | C₁₂H₂₅ | 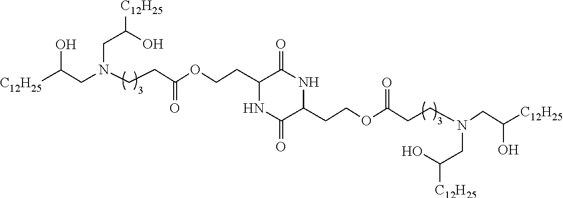 |
| 130 | 1 | C₁₄H₂₉ | 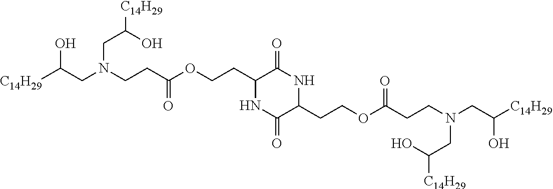 |
| 131 | 2 | C₁₄H₂₉ | 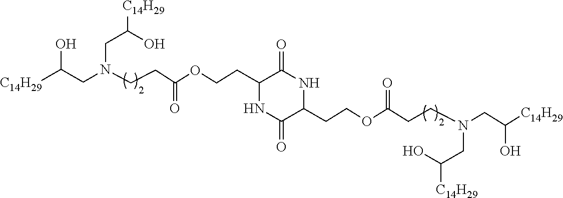 |
| 132 | 3 | C₁₄H₂₉ | 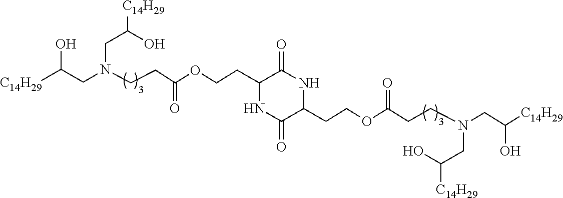 |
| 133 | 1 | C₁₆H₃₃ | 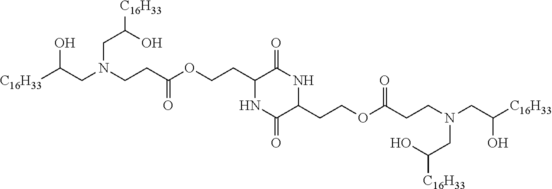 |
| 134 | 2 | C₁₆H₃₃ | 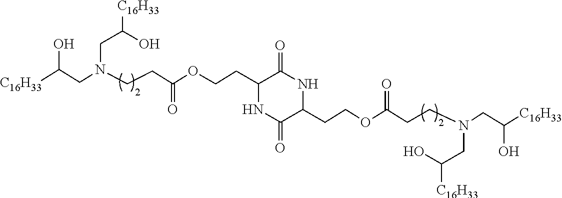 |
| 135 | 3 | C₁₆H₃₃ | 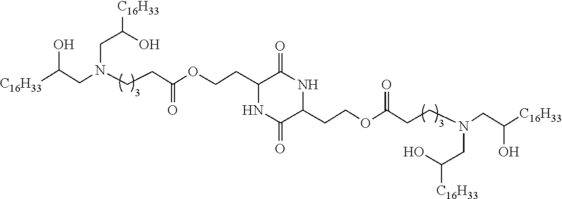 |
| 136 | 1 | C₁₆H₃₁ | 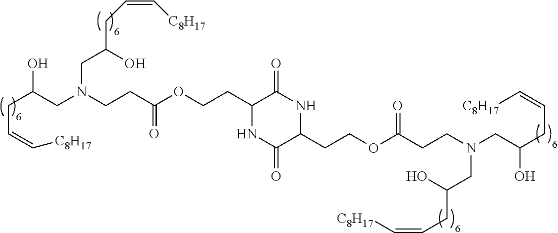 |
| 137 | 2 | C₁₆H₃₁ | 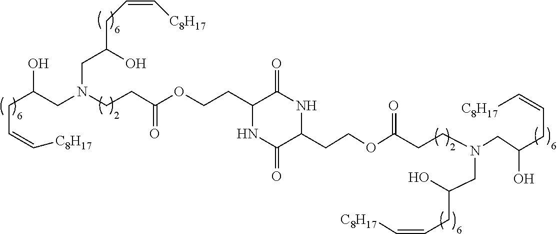 |
| 138 | 3 | C₁₆H₃₁ | 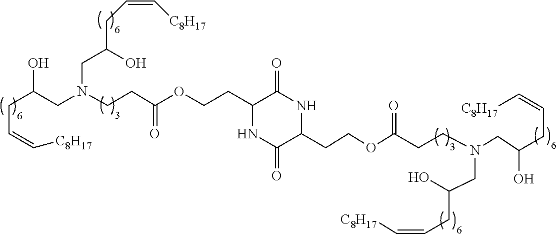 |
| 139 | 1 | C₁₆H₂₉ | 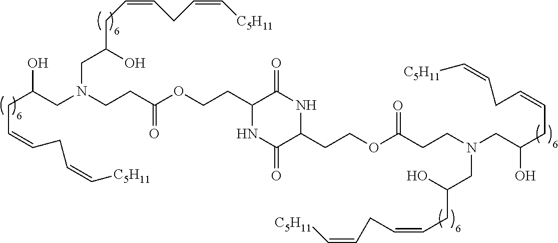 |
| 140 | 2 | C₁₆H₂₉ | 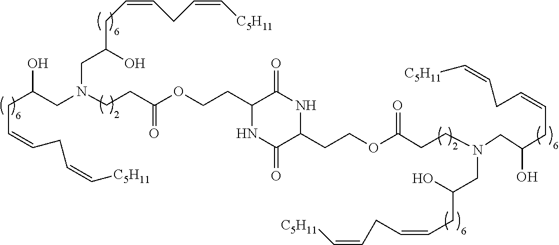 |
| 141 | 3 | C₁₆H₂₉ | 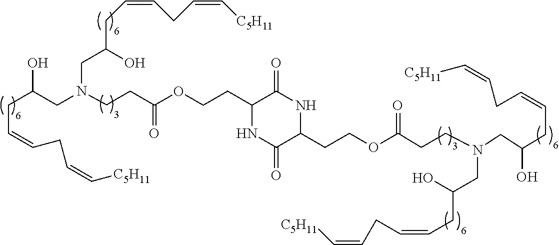 |
| 142 | 1 | C₆H₁₃ | 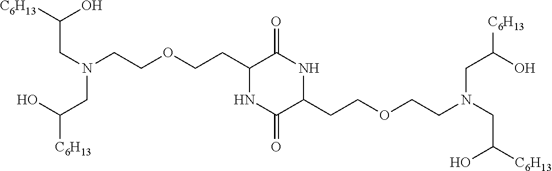 |
| 143 | 2 | C₆H₁₃ | 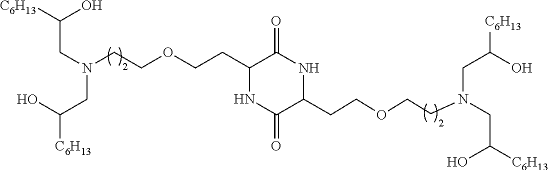 |
| 144 | 3 | C₆H₁₃ | 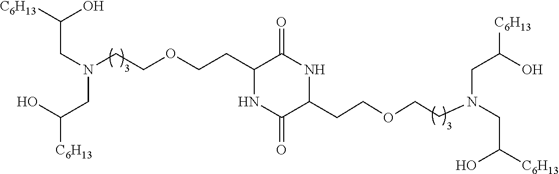 |
| 145 | 1 | substructure a | 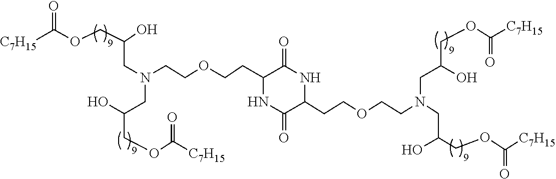 |
| 146 | 2 | substructure a | 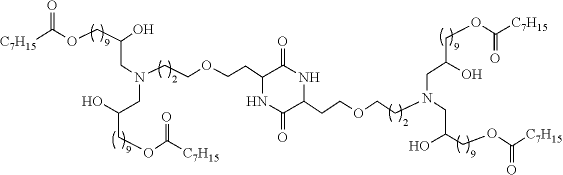 |
| 147 | 3 | substructure a | 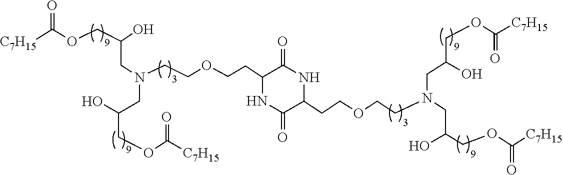 |
| 148 | 1 | substructure b | 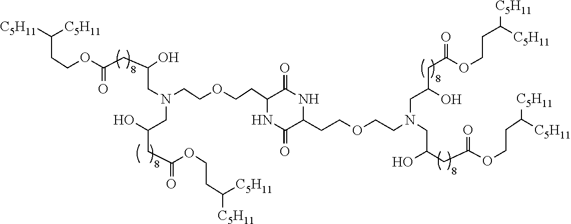 |
| 149 | 2 | substructure b | 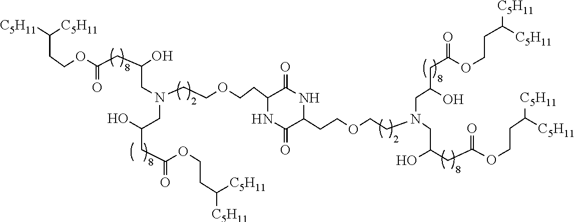 |
| 150 | 3 | substructure b | 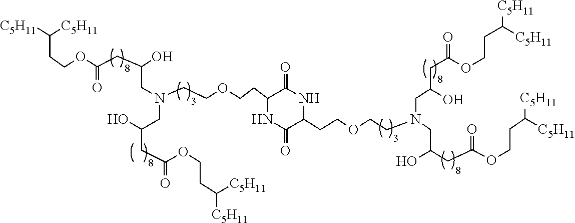 |

In embodiments, a cationic lipid is Compound 121. In embodiments, a cationic lipid is Compound 122. In embodiments, a cationic lipid is Compound 123. In embodiments, a cationic lipid is Compound 124. In embodiments, a cationic lipid is Compound 125. In embodiments, a cationic lipid is Compound 126. In embodiments, a cationic lipid is Compound 127. In embodiments, a cationic lipid is Compound 128. In embodiments, a cationic lipid is Compound 129. In embodiments, a cationic lipid is Compound 130. In embodiments, a cationic lipid is Compound 131. In embodiments, a cationic lipid is Compound 132. In embodiments, a cationic lipid is Compound 133. In embodiments, a cationic lipid is Compound 134. In embodiments, a cationic lipid is Compound 135. In embodiments, a cationic lipid is Compound 136. In embodiments, a cationic lipid is Compound 137. In embodiments, a cationic lipid is Compound 138. In embodiments, a cationic lipid is Compound 139. In embodiments, a cationic lipid is Compound 140. In embodiments, a cationic lipid is Compound 141. In embodiments, a cationic lipid is Compound 142. In embodiments, a cationic lipid is Compound 143. In embodiments, a cationic lipid is Compound 144. In embodiments, a cationic lipid is Compound 145. In embodiments, a cationic lipid is Compound 146. In embodiments, a cationic lipid is Compound 147. In embodiments, a cationic lipid is Compound 148. In embodiments, a cationic lipid is Compound 149. In embodiments, a cationic lipid is Compound 150.

TABLE F
cCC Disulfides

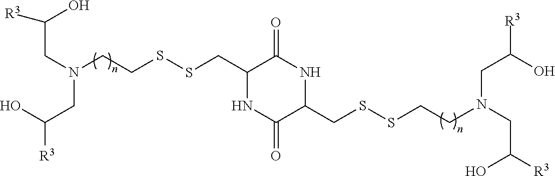

| No. | n | R³ | Structure |
|---|---|---|---|
| 151 | 1 | C₈H₁₇ | 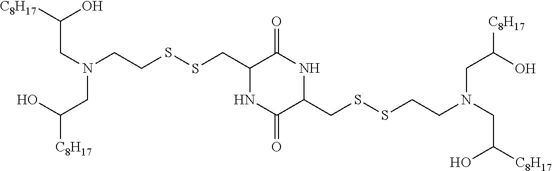 |
| 152 | 2 | C₈H₁₇ | 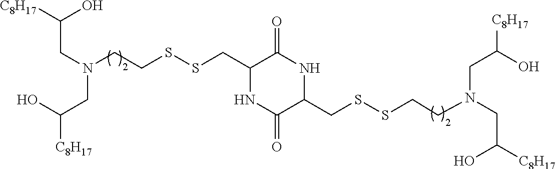 |
| 153 | 3 | C₈H₁₇ | 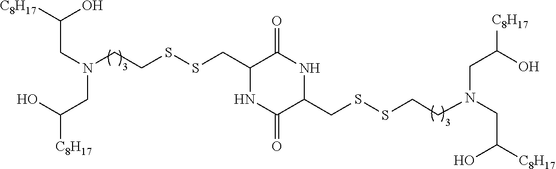 |
| 154 | 1 | C₁₀H₂₁ | 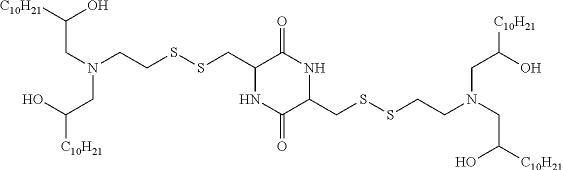 |
| 155 | 2 | C₁₀H₂₁ | 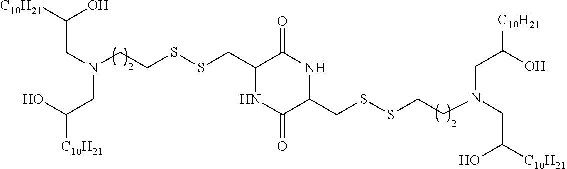 |
| 156 | 3 | C₁₀H₂₁ | 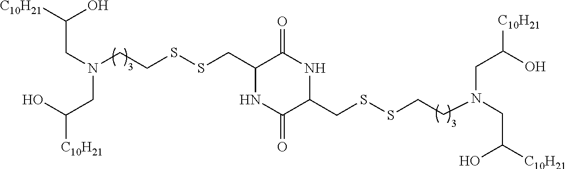 |
| 157 | 1 | C₁₂H₂₅ | 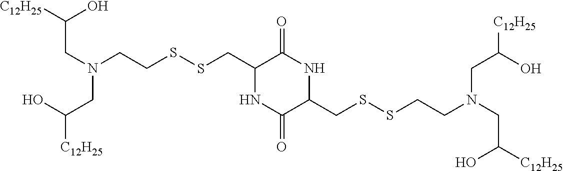 |
| 158 | 2 | C₁₂H₂₅ | 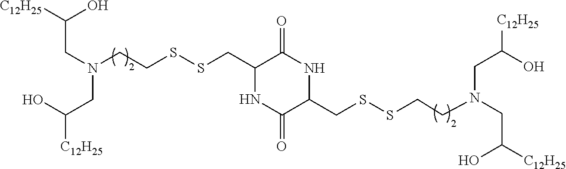 |
| 159 | 3 | C₁₂H₂₅ | 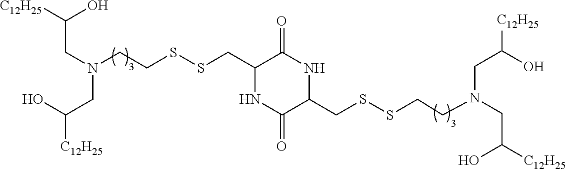 |
| 160 | 1 | C₁₄H₂₉ | 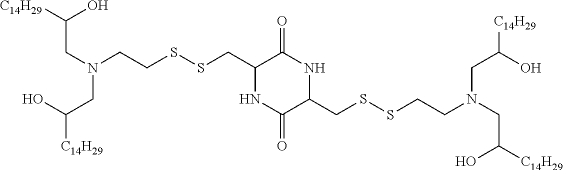 |
| 161 | 2 | C₁₄H₂₉ | 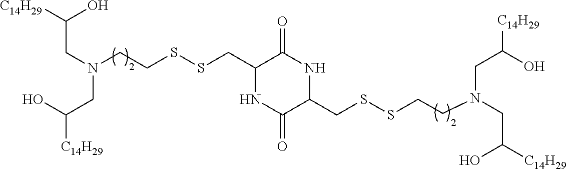 |
| 162 | 3 | C₁₄H₂₉ | 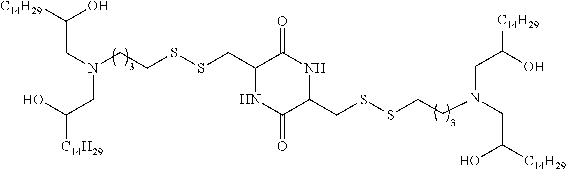 |
| 163 | 1 | C₁₆H₃₃ | 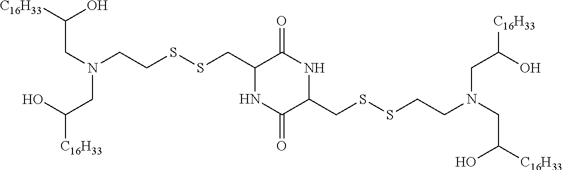 |
| 164 | 2 | C₁₆H₃₃ | 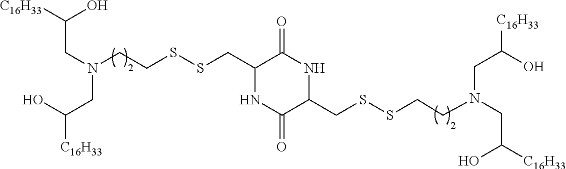 |
| 165 | 3 | C₁₆H₃₃ | 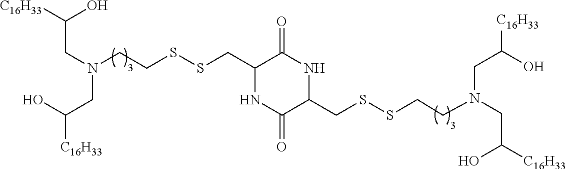 |
| 166 | 1 | C₁₆H₃₁ | 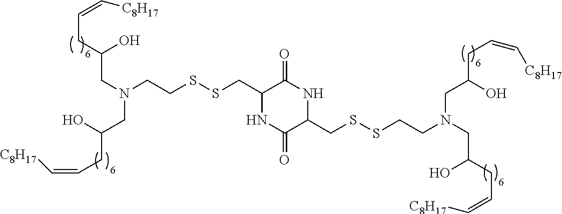 |
| 167 | 2 | C₁₆H₃₁ | 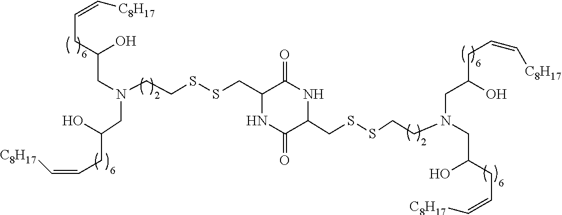 |
| 168 | 3 | C₁₆H₃₁ | 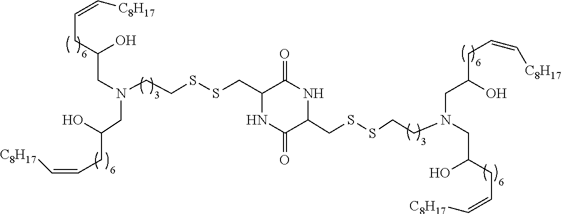 |
| 169 | 1 | C₁₆H₂₉ | 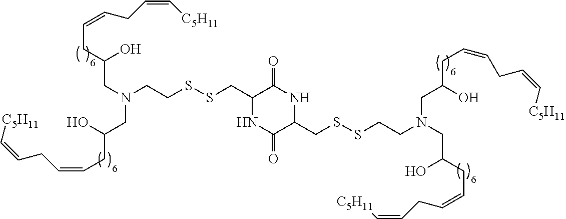 |
| 170 | 2 | C₁₆H₂₉ | 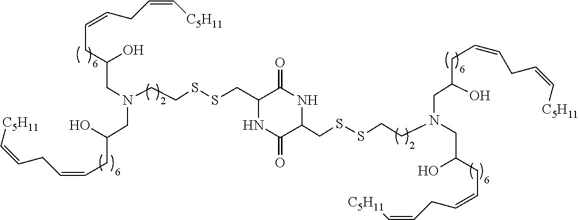 |
| 171 | 3 | C₁₆H₂₉ | 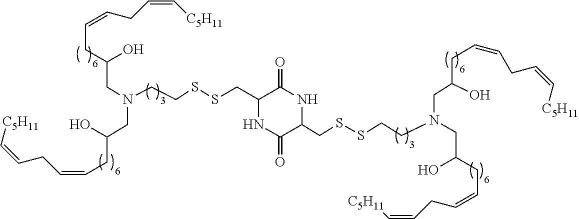 |
| 172 | 1 | C₆H₁₃ | 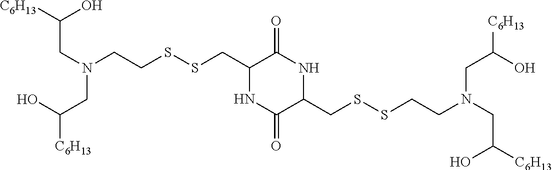 |
| 173 | 2 | C₆H₁₃ | 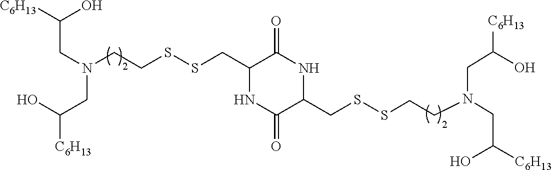 |
| 174 | 3 | C₆H₁₃ | 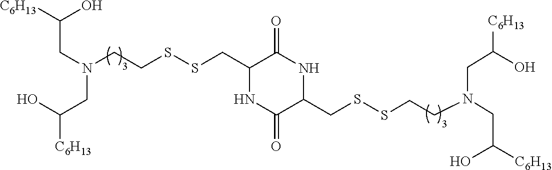 |
| 175 | 1 | substructure a | 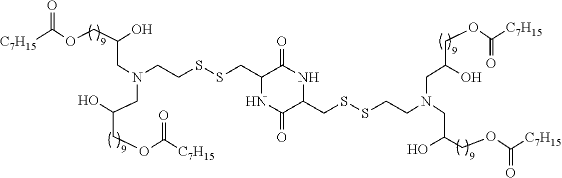 |
| 176 | 2 | substructure a | 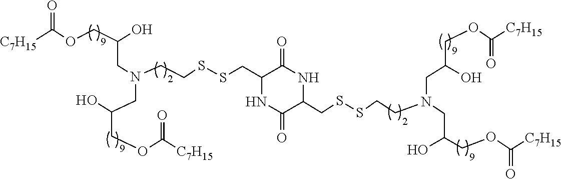 |
| 177 | 3 | substructure a | 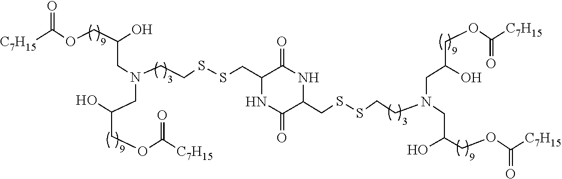 |
| 178 | 1 | substructure b | 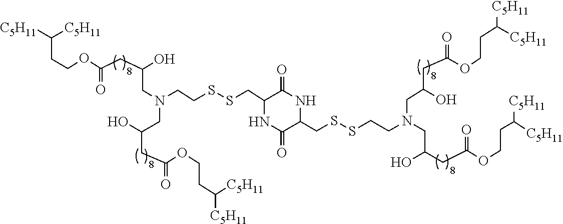 |
| 179 | 2 | substructure b | 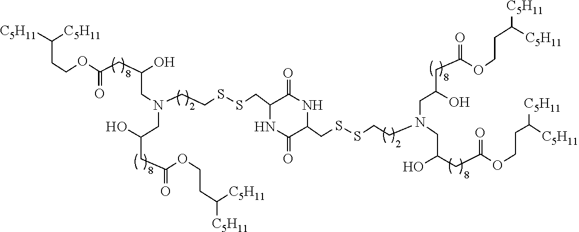 |
| 180 | 3 | substructure b | 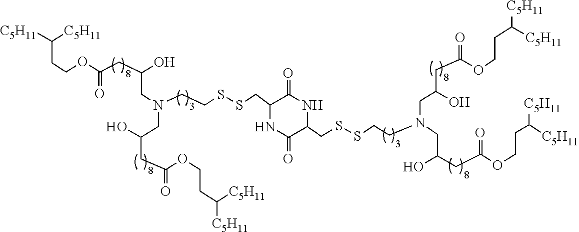 |

In embodiments, a cationic lipid is Compound 151. In embodiments, a cationic lipid is Compound 152. In embodiments, a cationic lipid is Compound 153. In embodiments, a cationic lipid is Compound 154. In embodiments, a cationic lipid is Compound 155. In embodiments, a cationic lipid is Compound 156. In embodiments, a cationic lipid is Compound 157. In embodiments, a cationic lipid is Compound 158. In embodiments, a cationic lipid is Compound 159. In embodiments, a cationic lipid is Compound 160. In embodiments, a cationic lipid is Compound 161. In embodiments, a cationic lipid is Compound 162. In embodiments, a cationic lipid is Compound 163. In embodiments, a cationic lipid is Compound 164. In embodiments, a cationic lipid is Compound 165. In embodiments, a cationic lipid is Compound 166. In embodiments, a cationic lipid is Compound 167. In embodiments, a cationic lipid is Compound 168. In embodiments, a cationic lipid is Compound 169. In embodiments, a cationic lipid is Compound 170. In embodiments, a cationic lipid is Compound 171. In embodiments, a cationic lipid is Compound 172. In embodiments, a cationic lipid is Compound 173. In embodiments, a cationic lipid is Compound 174. In embodiments, a cationic lipid is Compound 175. In embodiments, a cationic lipid is Compound 176. In embodiments, a cationic lipid is Compound 177. In embodiments, a cationic lipid is Compound 178. In embodiments, a cationic lipid is Compound 179. In embodiments, a cationic lipid is Compound 180.

TABLE G
cCC Thioesters

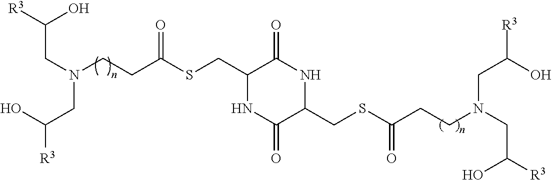

| No. | n | R³ | Structure |
|---|---|---|---|
| 181 | 1 | C₈H₁₇ | 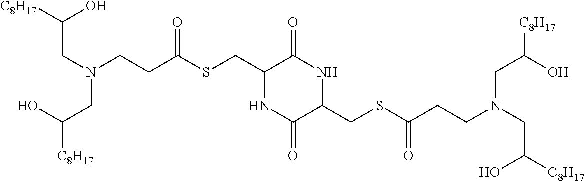 |
| 182 | 2 | C₈H₁₇ | 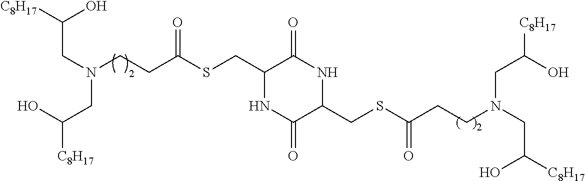 |
| 183 | 3 | C₈H₁₇ | 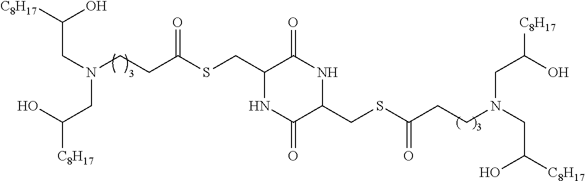 |
| 184 | 1 | C₁₀H₂₁ | 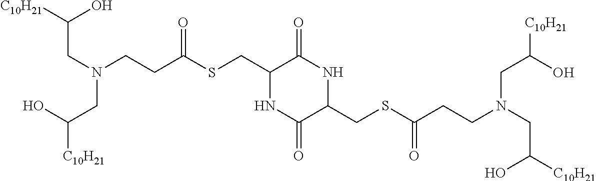 |
| 185 | 2 | C₁₀ H₂₁ | 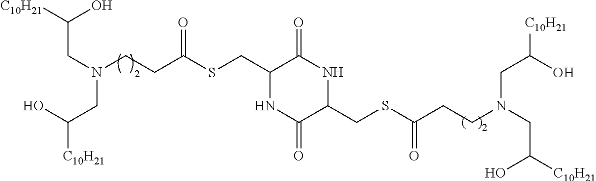 |
| 186 | 3 | C₁₀H₂₁ | 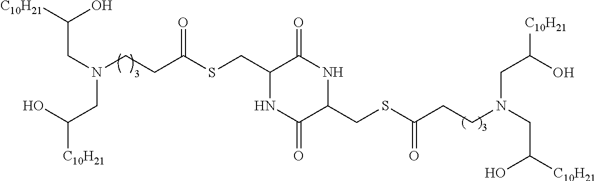 |
| 187 | 1 | C₈H₁₇ | 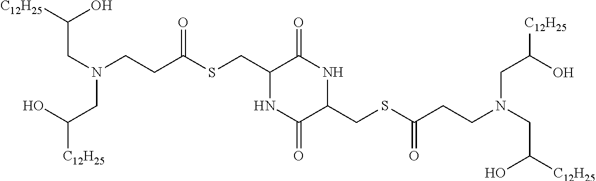 |
| 188 | 2 | C₁₂H₂₅ | 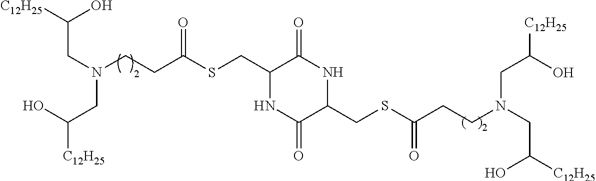 |
| 189 | 3 | C₁₂H₂₅ | 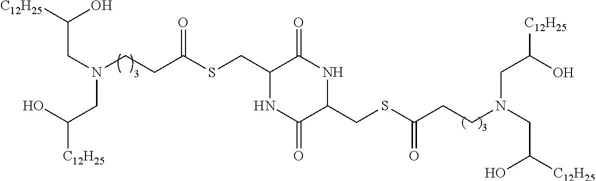 |
| 190 | 1 | C₁₄H₂₉ | 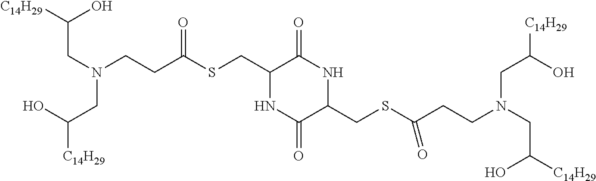 |
| 191 | 2 | C₁₄H₂₉ | 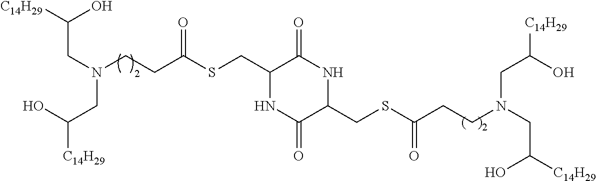 |
| 192 | 3 | C₁₄H₂₉ | 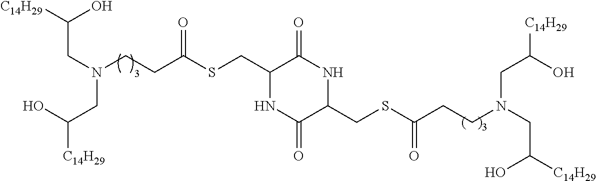 |
| 193 | 1 | C₁₆H₃₃ | 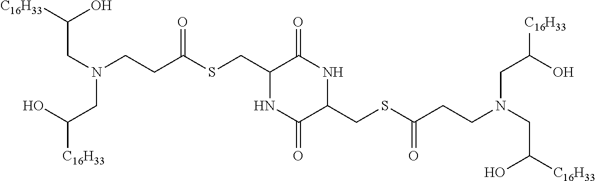 |
| 194 | 2 | C₁₆H₃₃ | 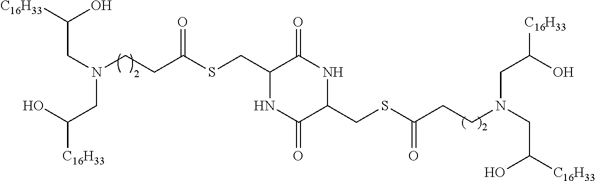 |
| 195 | 3 | C₁₆H₃₃ | 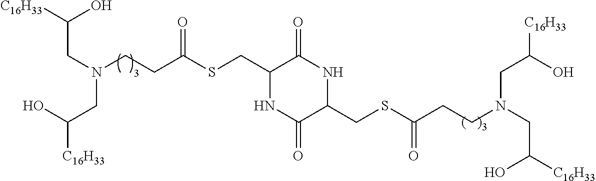 |
| 196 | 1 | C₁₆H₃₁ | 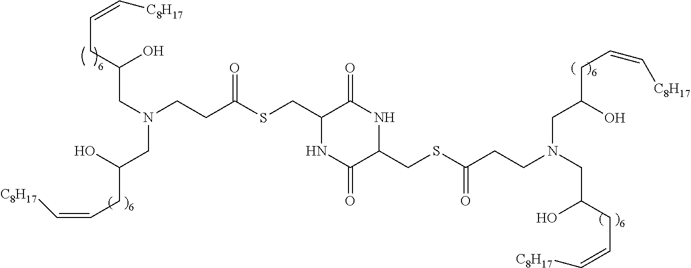 |
| 197 | 2 | C₁₆H₃₁ | 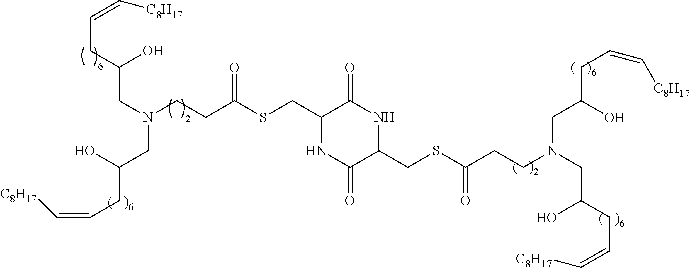 |
| 198 | 3 | C₁₆H₃₁ | 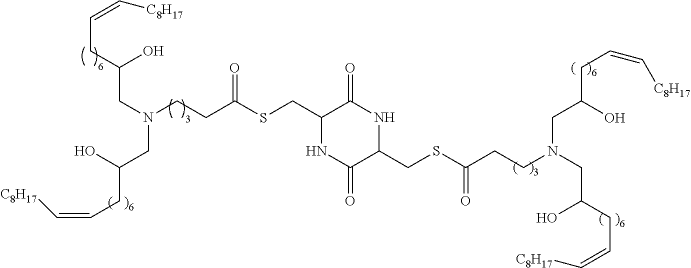 |
| 199 | 1 | C₁₆H₂₉ | 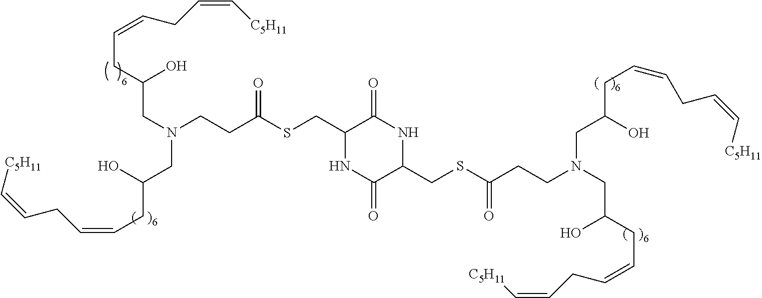 |
| 200 | 2 | C₁₆H₂₉ | 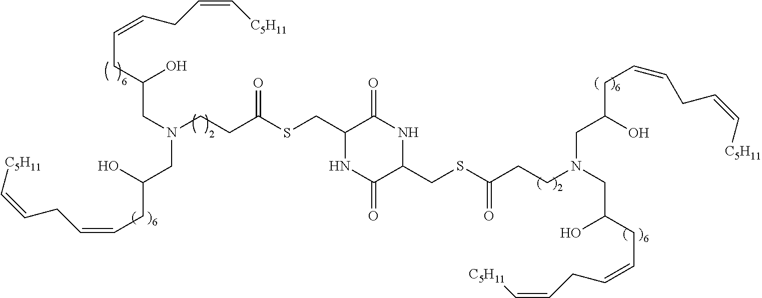 |
| 201 | 3 | C₁₆H₂₉ | 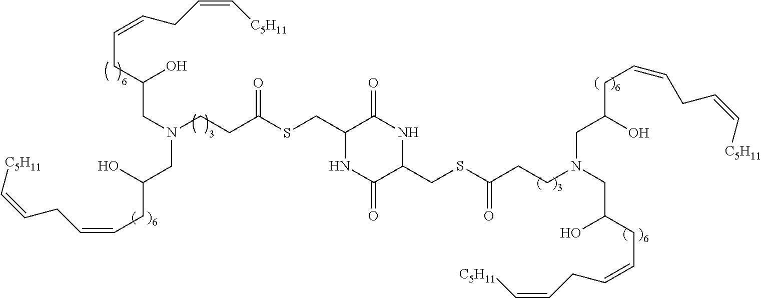 |
| 202 | 1 | C₆H₁₃ | 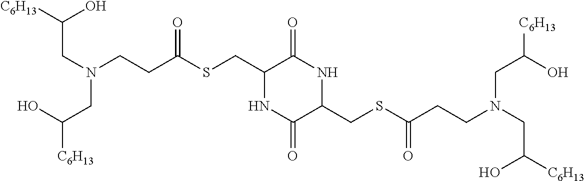 |
| 203 | 2 | C₆H₁₃ | 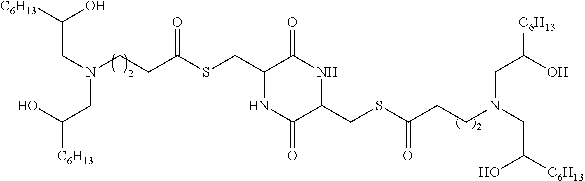 |
| 204 | 3 | C₆H₁₃ | 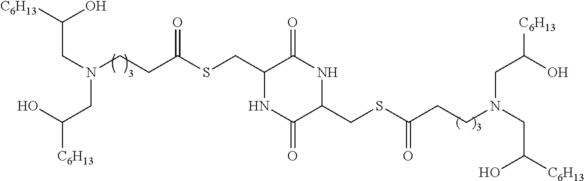 |
| 205 | 1 | substructure a | 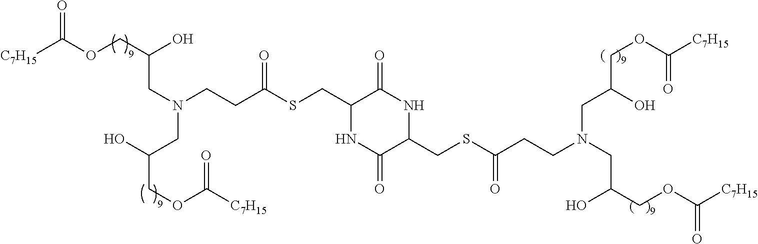 |
| 206 | 2 | substructure a | 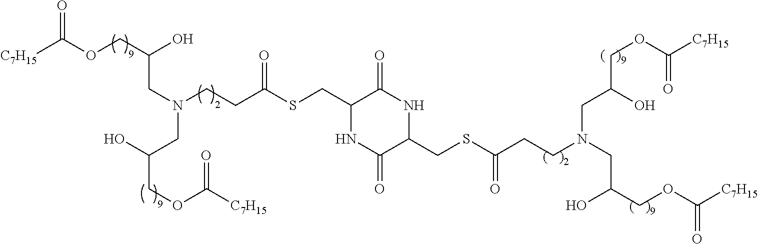 |
| 207 | 3 | substructure a | 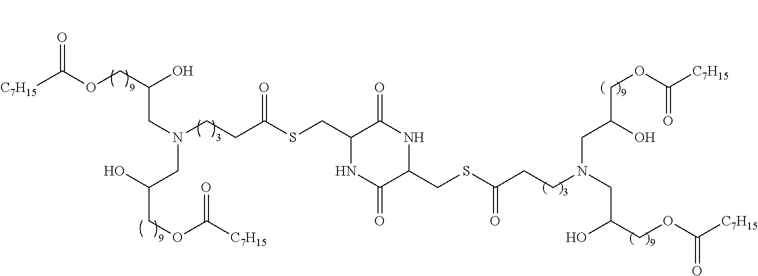 |
| 208 | 1 | substructure b | 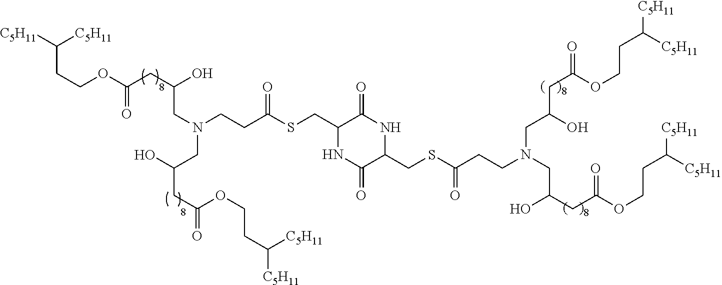 |
| 209 | 2 | substructure b | 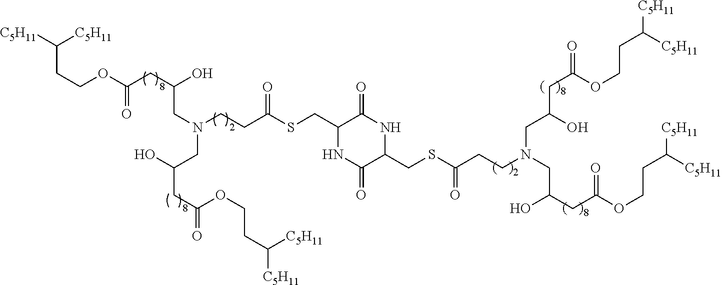 |
| 210 | 3 | substructure b | 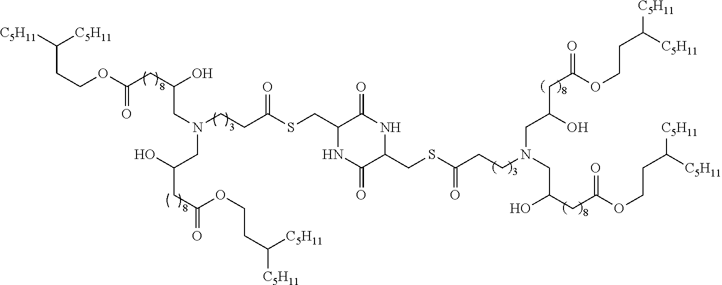 |

In embodiments, a cationic lipid is Compound 181. In embodiments, a cationic lipid is Compound 182. In embodiments, a cationic lipid is Compound 183. In embodiments, a cationic lipid is Compound 184. In embodiments, a cationic lipid is Compound 185. In embodiments, a cationic lipid is Compound 186. In embodiments, a cationic lipid is Compound 187. In embodiments, a cationic lipid is Compound 188. In embodiments, a cationic lipid is Compound 189. In embodiments, a cationic lipid is Compound 190. In embodiments, a cationic lipid is Compound 191. In embodiments, a cationic lipid is Compound 192. In embodiments, a cationic lipid is Compound 193. In embodiments, a cationic lipid is Compound 194. In embodiments, a cationic lipid is Compound 195. In embodiments, a cationic lipid is Compound 196. In embodiments, a cationic lipid is Compound 197. In embodiments, a cationic lipid is Compound 198. In embodiments, a cationic lipid is Compound 199. In embodiments, a cationic lipid is Compound 200. In embodiments, a cationic lipid is Compound 201. In embodiments, a cationic lipid is Compound 202. In embodiments, a cationic lipid is Compound 203. In embodiments, a cationic lipid is Compound 204. In embodiments, a cationic lipid is Compound 205. In embodiments, a cationic lipid is Compound 206. In embodiments, a cationic lipid is Compound 207. In embodiments, a cationic lipid is Compound 208. In embodiments, a cationic lipid is Compound 209. In embodiments, a cationic lipid is Compound 210.

Table H
cSS Esters

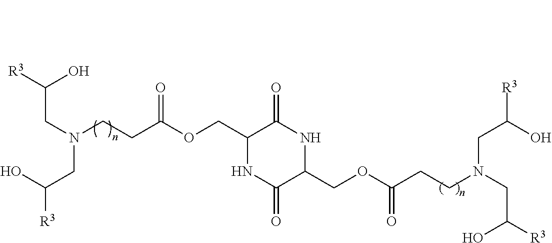

| No. | n | R³ | Structure |
|---|---|---|---|
| 211 | 1 | C₈H₁₇ | 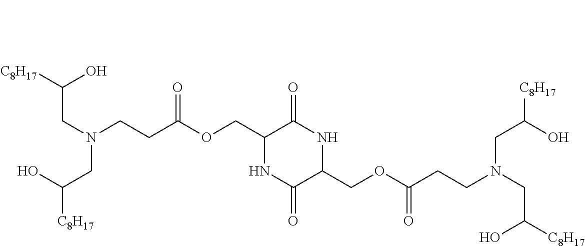 |
| 212 | 2 | C₈H₁₇ | 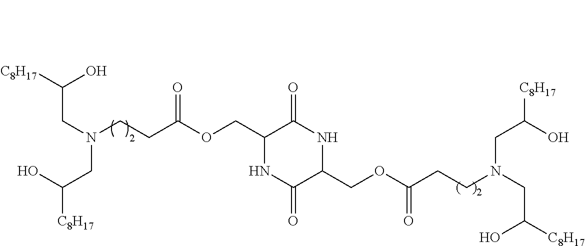 |
| 213 | 3 | C₈H₁₇ | 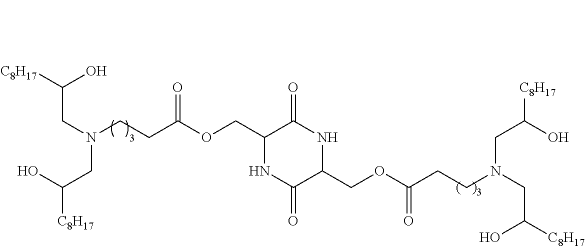 |
| 214 | 1 | C₁₀H₂₁ | 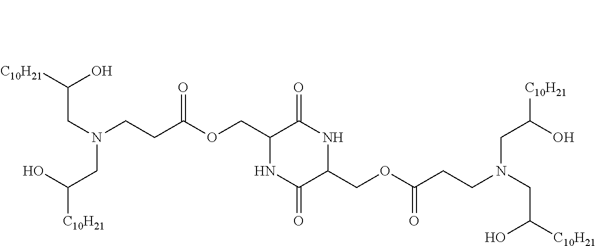 |
| 215 | 2 | C₁₀H₂₁ | 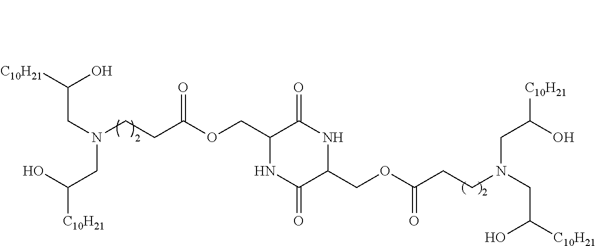 |
| 216 | 3 | C₁₀H₂₁ | 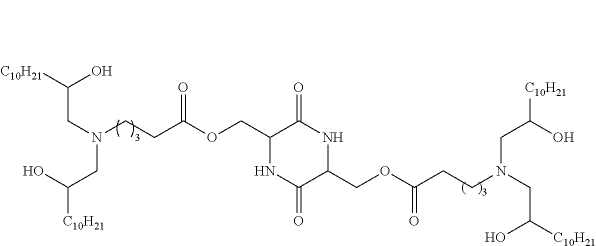 |
| 217 | 1 | C₁₂H₂₅ | 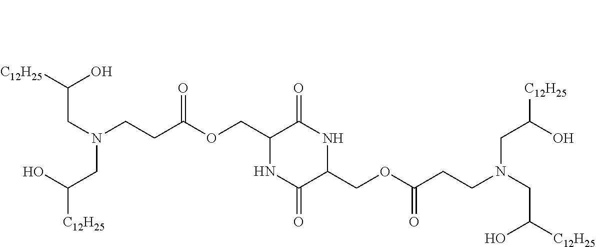 |
| 218 | 2 | C₁₂H₂₅ | 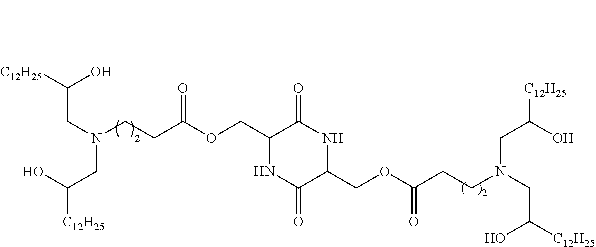 |
| 219 | 3 | C₁₂H₂₅ | 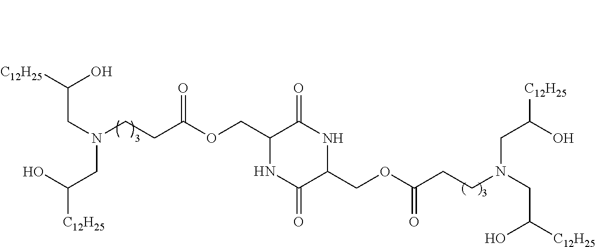 |
| 220 | 1 | C₁₄H₂₉ | 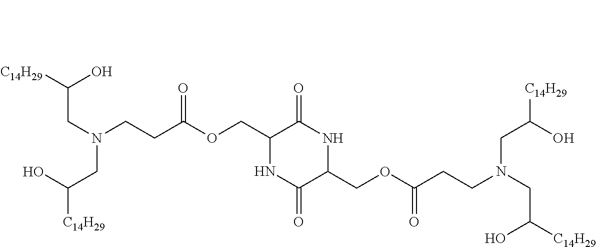 |
| 221 | 2 | C₁₄H₂₉ | 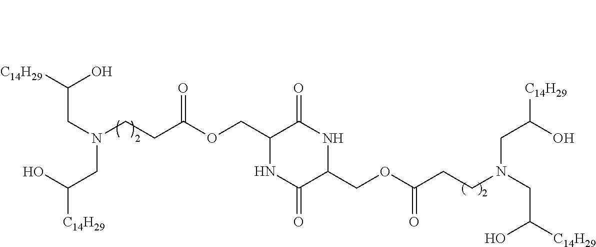 |
| 222 | 3 | C₁₄H₂₉ | 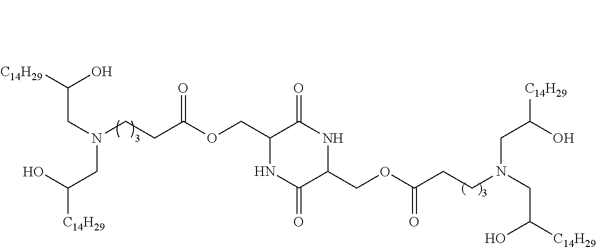 |
| 223 | 1 | C₁₆H₃₃ | 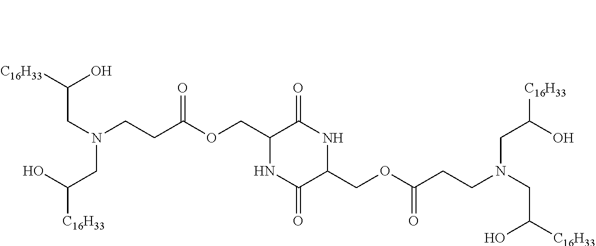 |
| 224 | 2 | C₁₆H₃₃ | 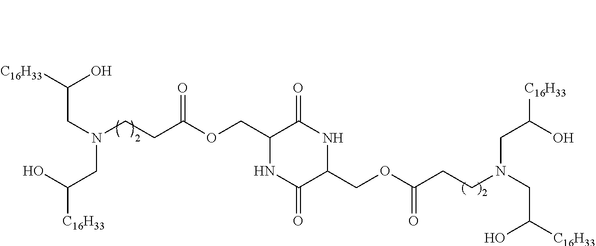 |
| 225 | 3 | C₁₆H₃₃ | 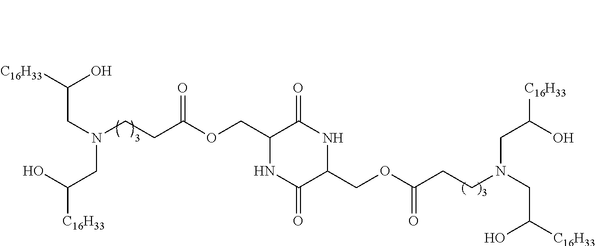 |
| 226 | 1 | C₁₆H₃₃ | 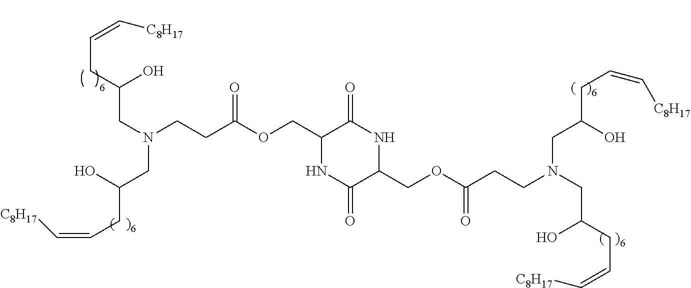 |
| 227 | 2 | C₁₆H₃₁ | 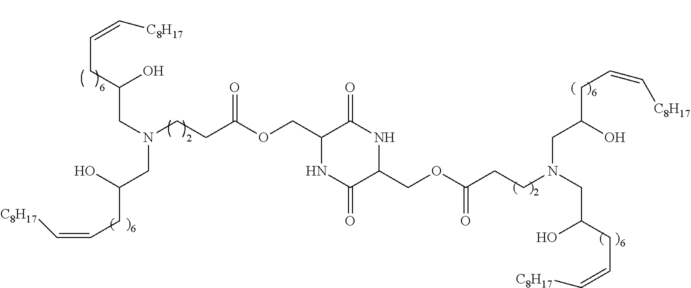 |
| 228 | 3 | C₁₆H₃₁ | 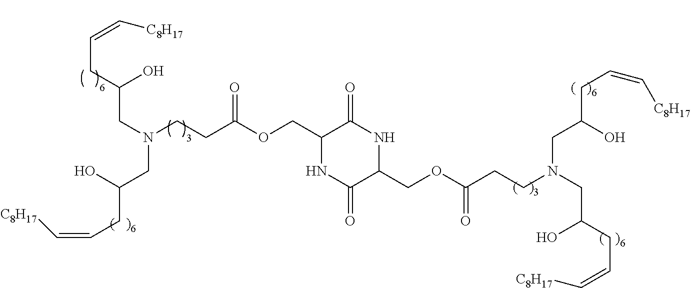 |
| 229 | 1 | C₁₆H₂₉ | 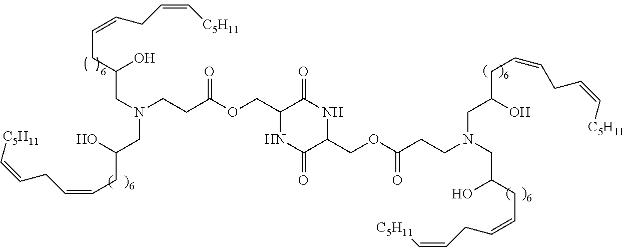 |
| 230 | 2 | C₁₆H₃₃ | 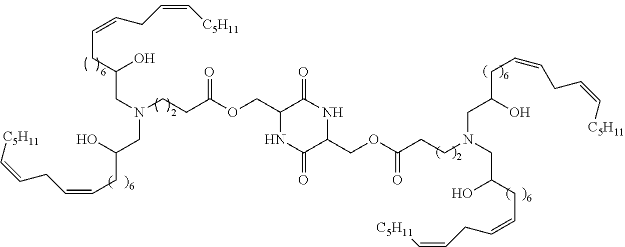 |
| 231 | 3 | C₁₆H₂₉ | 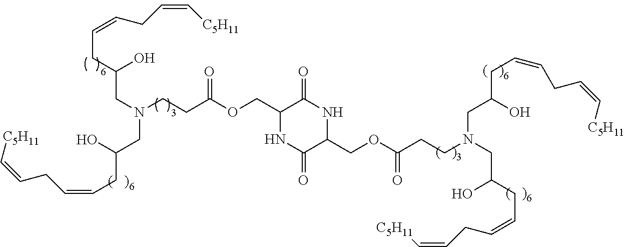 |
| 232 | 1 | C₆H₁₃ | 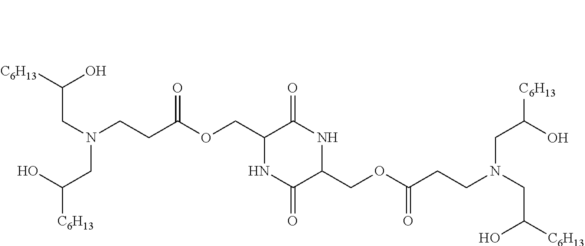 |
| 233 | 2 | C₆H₁₃ | 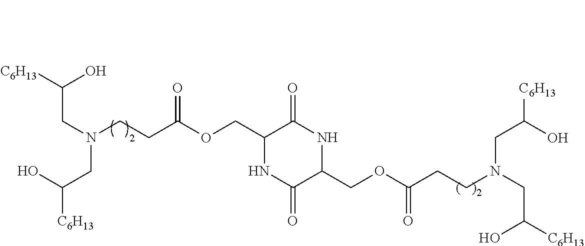 |
| 234 | 3 | C₆H₁₃ | 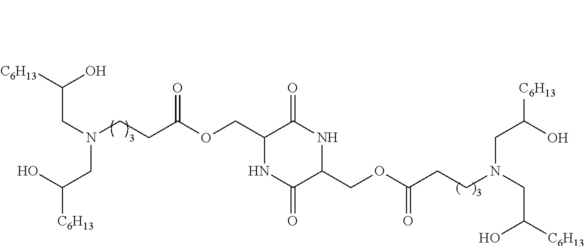 |
| 235 | 1 | substructure a | 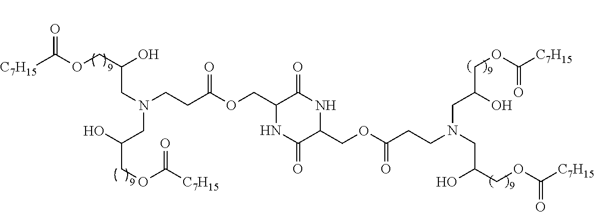 |
| 236 | 2 | substructure a | 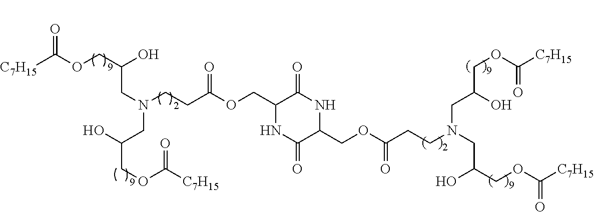 |
| 237 | 3 | substructure a | 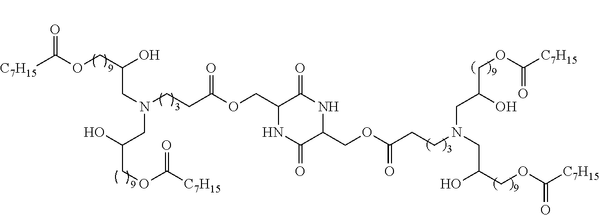 |
| 238 | 1 | substructure b | 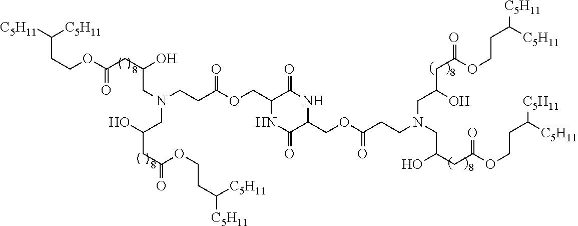 |
| 239 | 2 | substructure b | 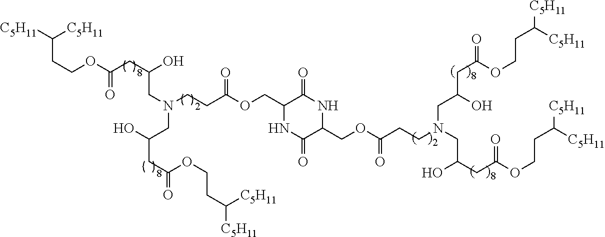 |
| 240 | 3 | substructure b | 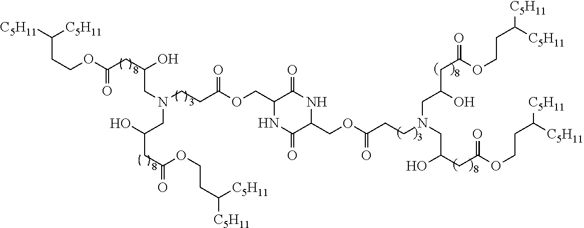 |

In embodiments, a cationic lipid is Compound 211. In embodiments, a cationic lipid is Compound 212. In embodiments, a cationic lipid is Compound 213. In embodiments, a cationic lipid is Compound 214. In embodiments, a cationic lipid is Compound 215. In embodiments, a cationic lipid is Compound 216. In embodiments, a cationic lipid is Compound 217. In embodiments, a cationic lipid is Compound 218. In embodiments, a cationic lipid is Compound 219. In embodiments, a cationic lipid is Compound 220. In embodiments, a cationic lipid is Compound 221. In embodiments, a cationic lipid is Compound 222. In embodiments, a cationic lipid is Compound 223. In embodiments, a cationic lipid is Compound 224. In embodiments, a cationic lipid is Compound 225. In embodiments, a cationic lipid is Compound 226. In embodiments, a cationic lipid is Compound 227. In embodiments, a cationic lipid is Compound 228. In embodiments, a cationic lipid is Compound 229. In embodiments, a cationic lipid is Compound 230. In embodiments, a cationic lipid is Compound 231. In embodiments, a cationic lipid is Compound 232. In embodiments, a cationic lipid is Compound 233. In embodiments, a cationic lipid is Compound 234. In embodiments, a cationic lipid is Compound 235. In embodiments, a cationic lipid is Compound 236. In embodiments, a cationic lipid is Compound 237. In embodiments, a cationic lipid is Compound 238. In embodiments, a cationic lipid is Compound 239. In embodiments, a cationic lipid is Compound 240.

TABLE I
cDD Thioesters - Biodegradable

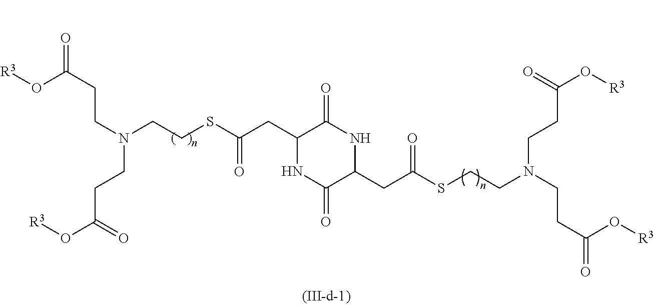

| No. | n | R³ | Structure |
|---|---|---|---|
| 241 | 1 | C₆H₁₃ | 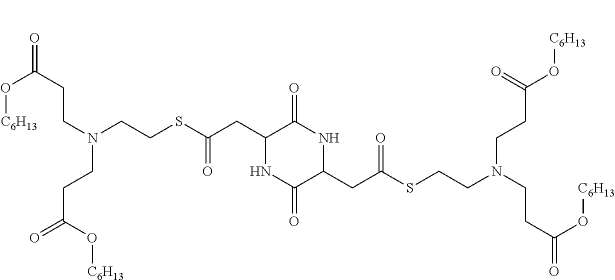 |
| 242 | 2 | C₆H₁₃ | 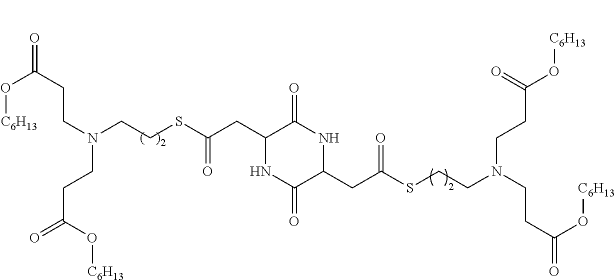 |
| 243 | 3 | C₆H₁₃ | 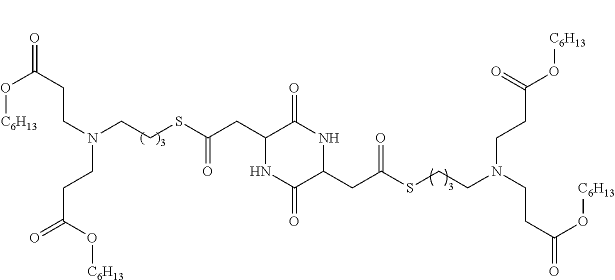 |
| 244 | 1 | C₈H₁₇ | 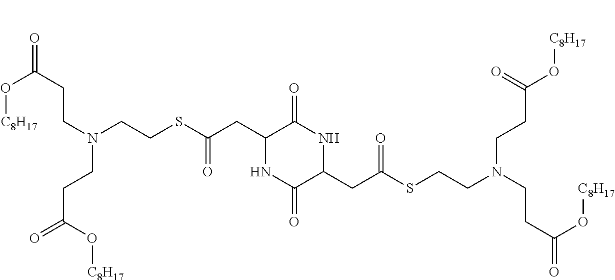 |
| 245 | 2 | C₁₆H₃₃ | 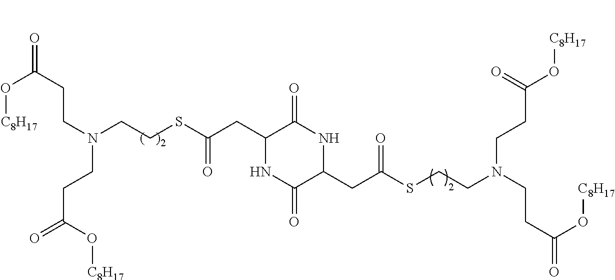 |
| 246 | 3 | C₈H₁₇ | 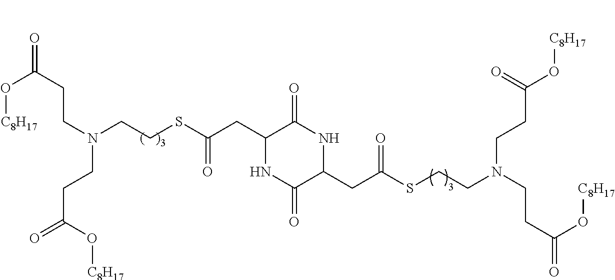 |
| 247 | 1 | C₁₀H₂₁ | 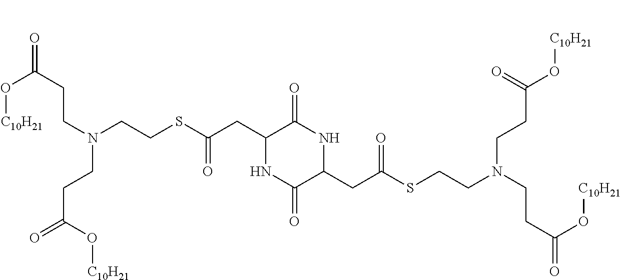 |
| 248 | 2 | C₁₀H₂₁ | 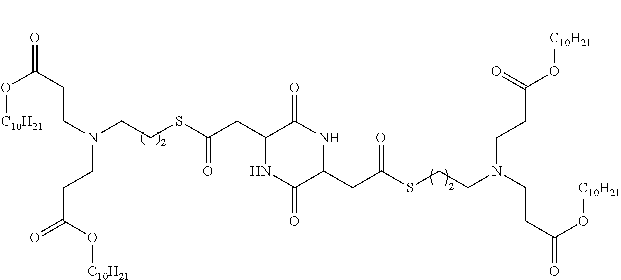 |
| 249 | 3 | C₁₀H₂₁ | 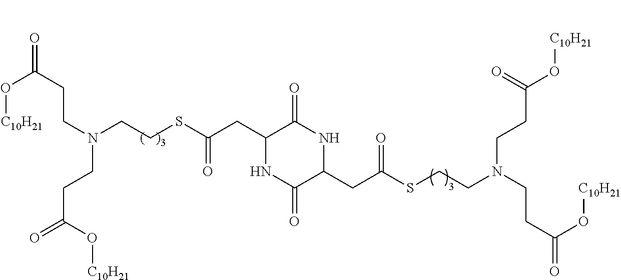 |
| 250 | 1 | C₁₂H₂₅ | 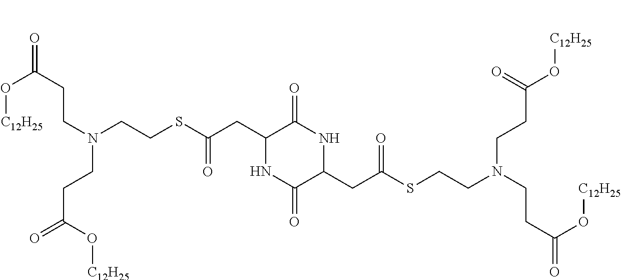 |
| 251 | 2 | C₁₂H₂₅ | 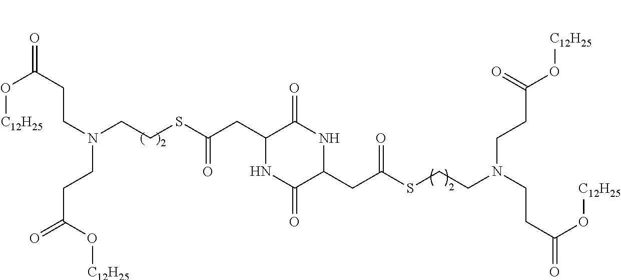 |
| 252 | 3 | C₁₂H₂₅ | 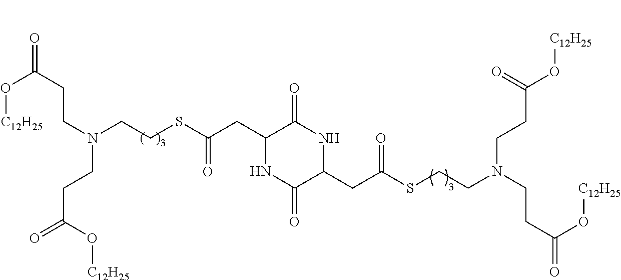 |
| 253 | 1 | C₁₄H₂₉ | 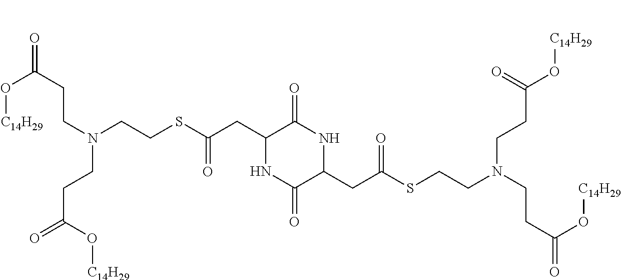 |
| 254 | 2 | C₁₄H₂₉ | 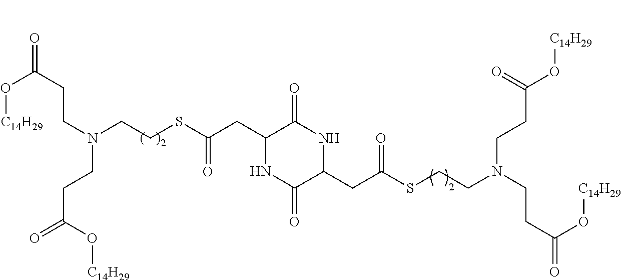 |
| 255 | 3 | C₁₄H₂₉ | 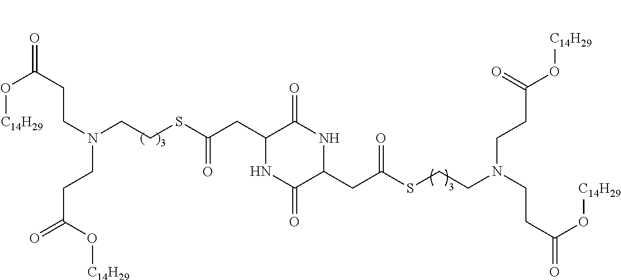 |
| 256 | 1 | C₁₆H₃₃ | 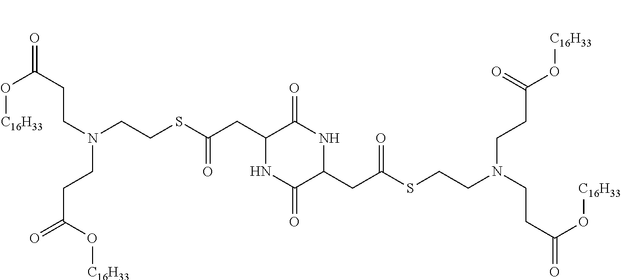 |
| 257 | 2 | C₁₆H₃₃ | 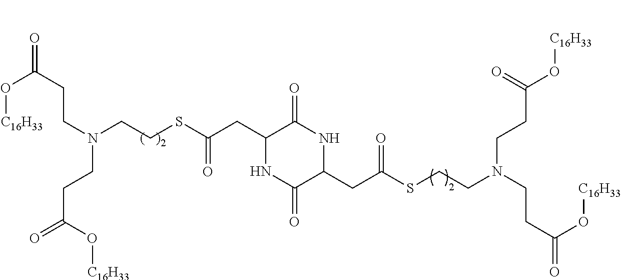 |
| 258 | 3 | C₁₆H₃₃ | 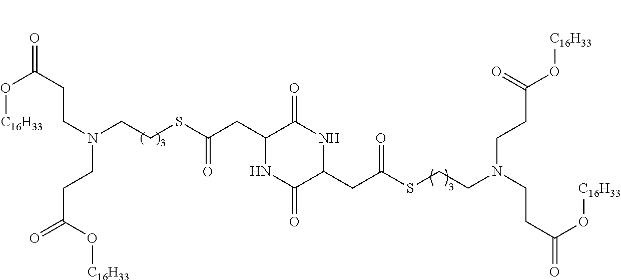 |
| 259 | 1 | C₁₈H₃₇ | 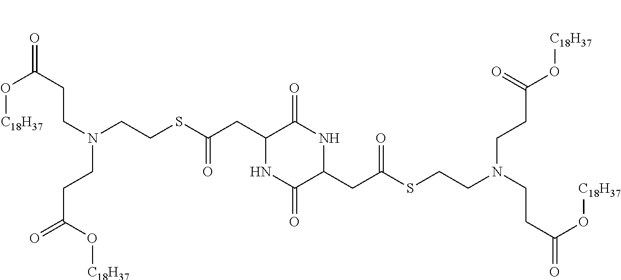 |
| 260 | 2 | C₁₈H₃₇ | 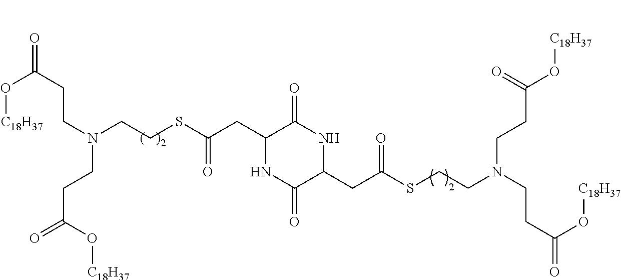 |
| 261 | 3 | C₁₈H₃₇ | 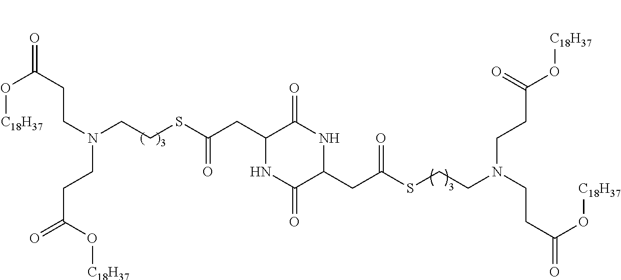 |
| 262 | 1 | C₁₈H₃₃ | 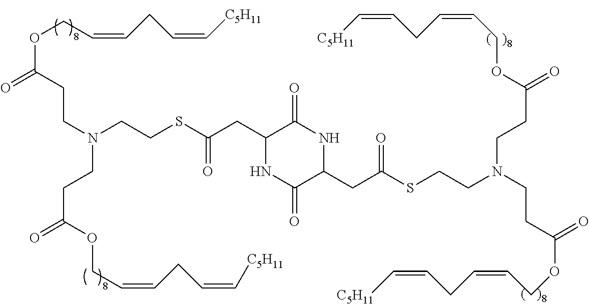 |
| 263 | 2 | C₁₈H₃₃ | 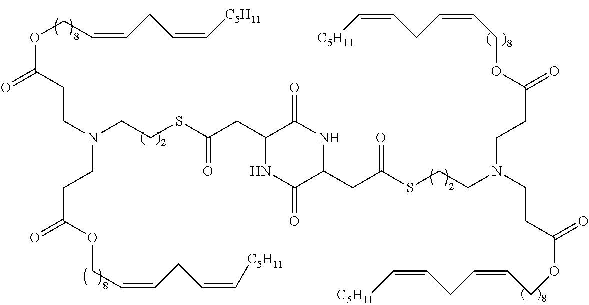 |
| 264 | 3 | C₁₈H₃₃ | 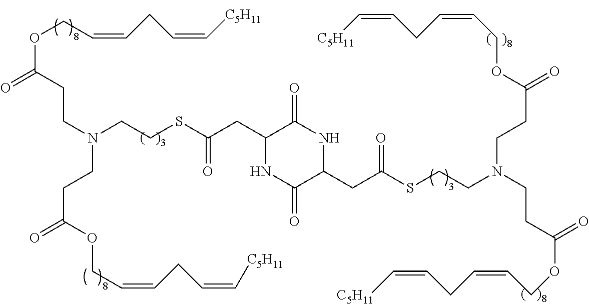 |
| 265 | 1 | C₁₈H₃₅ | 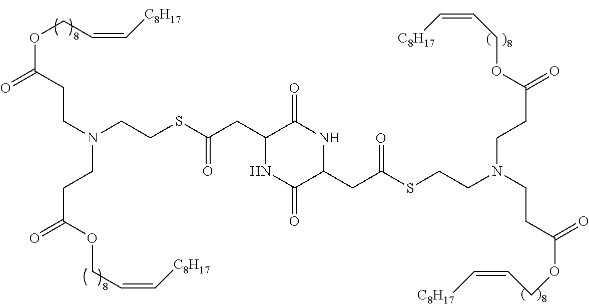 |
| 266 | 2 | C₁₈H₃₅ | 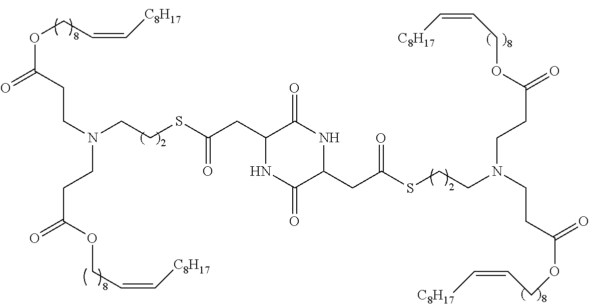 |
| 267 | 3 | C₁₈H₃₅ | 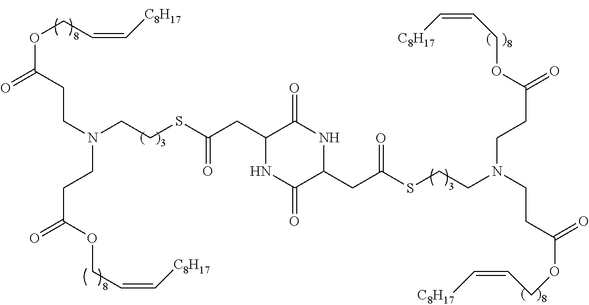 |
| 268 | 1 | C₁₈H₃₁ | 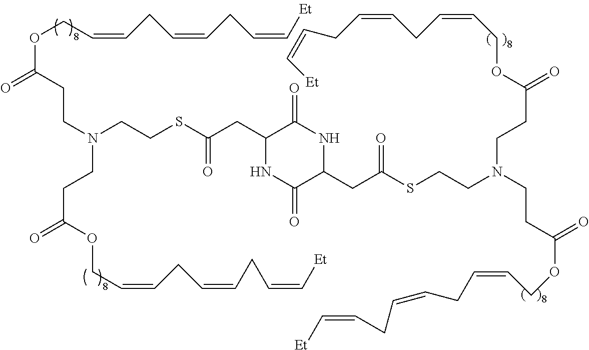 |
| 269 | 2 | C₁₈H₃₁ | 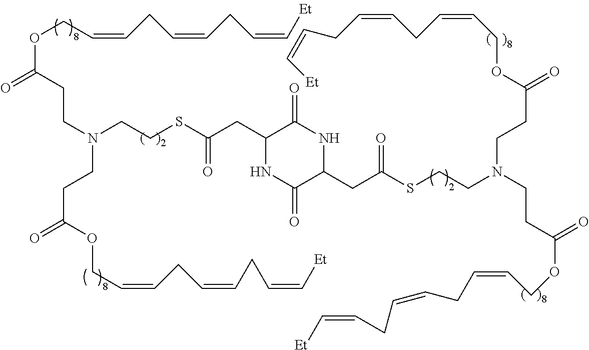 |
| 270 | 3 | C₁₈H₃₁ | 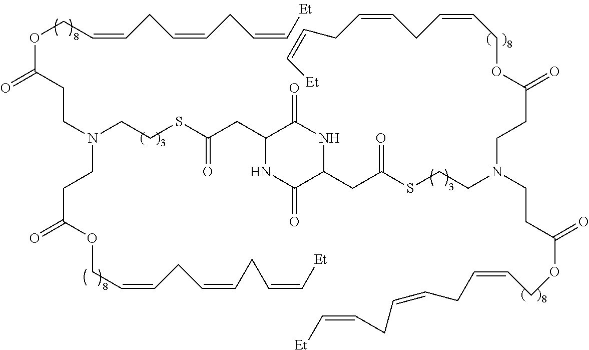 |
| 271 | 1 | substructure a | 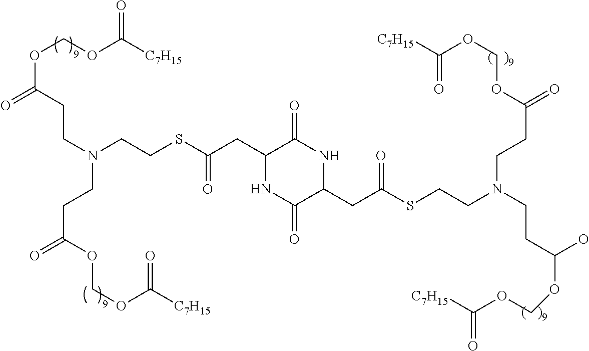 |
| 272 | 2 | substructure a | 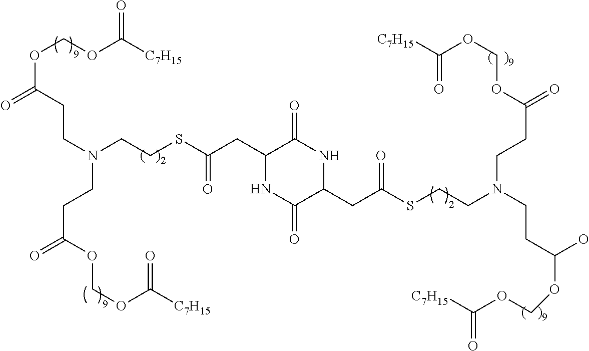 |
| 273 | 3 | substructure a | 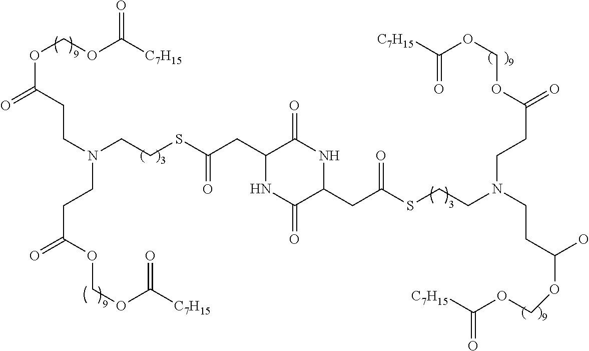 |
| 274 | 1 | substructure b | 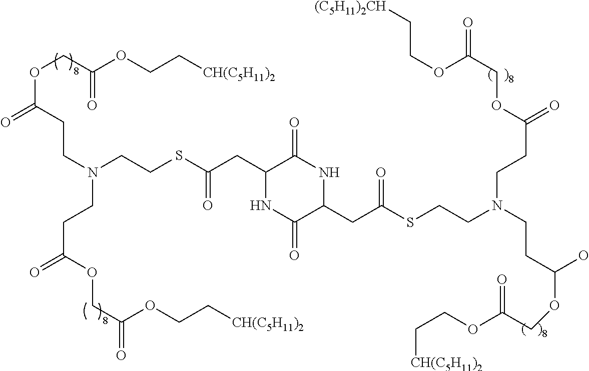 |
| 275 | 2 | substructure b | 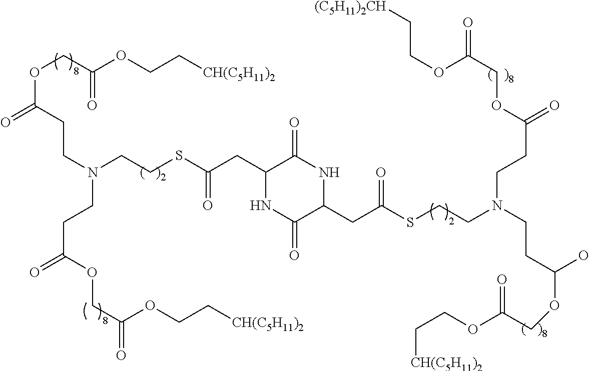 |
| 276 | 3 | substructure b | 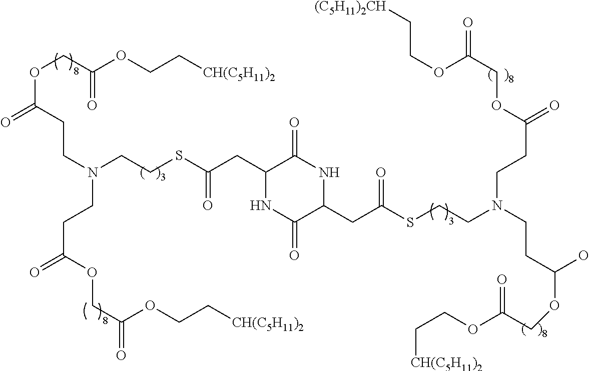 |
| 277 | 1 | —(C₇H₁₄)— CH(CH₃)₂ | 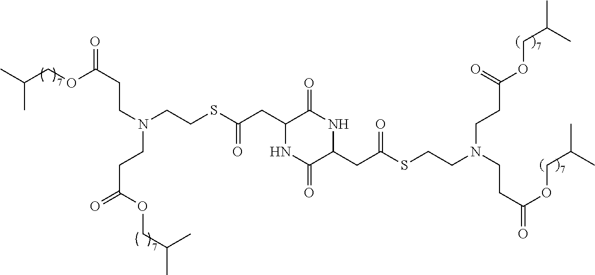 |
| 278 | 2 | —(C₇H₁₄)— CH(CH₃)₂ | 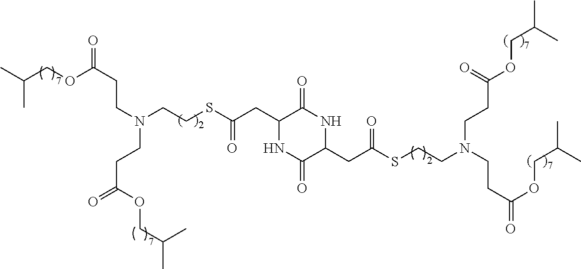 |
| 279 | 3 | —(C₇H₁₄)— CH(CH₃)₂ | 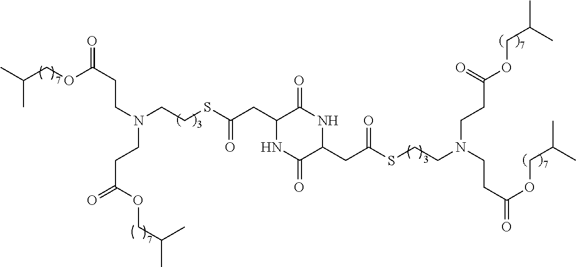 |

In embodiments, a cationic lipid is Compound 241. In embodiments, a cationic lipid is Compound 242. In embodiments, a cationic lipid is Compound 243. In embodiments, a cationic lipid is Compound 244. In embodiments, a cationic lipid is Compound 245. In embodiments, a cationic lipid is Compound 246. In embodiments, a cationic lipid is Compound 247. In embodiments, a cationic lipid is Compound 248. In embodiments, a cationic lipid is Compound 249. In embodiments, a cationic lipid is Compound 250. In embodiments, a cationic lipid is Compound 251. In embodiments, a cationic lipid is Compound 252. In embodiments, a cationic lipid is Compound 253. In embodiments, a cationic lipid is Compound 254. In embodiments, a cationic lipid is Compound 255. In embodiments, a cationic lipid is Compound 256. In embodiments, a cationic lipid is Compound 257. In embodiments, a cationic lipid is Compound 258. In embodiments, a cationic lipid is Compound 259. In embodiments, a cationic lipid is Compound 260. In embodiments, a cationic lipid is Compound 261. In embodiments, a cationic lipid is Compound 262. In embodiments, a cationic lipid is Compound 263. In embodiments, a cationic lipid is Compound 264. In embodiments, a cationic lipid is Compound 265. In embodiments, a cationic lipid is Compound 266. In embodiments, a cationic lipid is Compound 267. In embodiments, a cationic lipid is Compound 268. In embodiments, a cationic lipid is Compound 269. In embodiments, a cationic lipid is Compound 270. In embodiments, a cationic lipid is Compound 271. In embodiments, a cationic lipid is Compound 272. In embodiments, a cationic lipid is Compound 273. In embodiments, a cationic lipid is Compound 274. In embodiments, a cationic lipid is Compound 275. In embodiments, a cationic lipid is Compound 276. In embodiments, a cationic lipid is Compound 277. In embodiments, a cationic lipid is Compound 278. In embodiments, a cationic lipid is Compound 279.

TABLE J
cDD Esters- Biodegradable

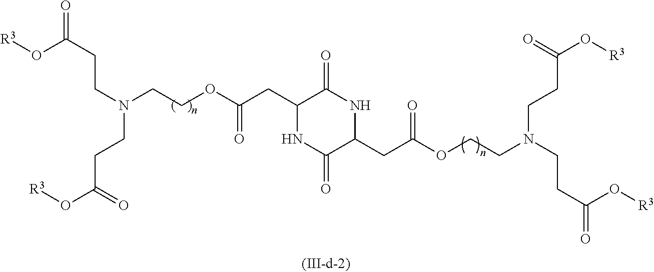

| No. | n | R³ | Structure |
|---|---|---|---|
| 280 | 1 | C₆H₁₃ | 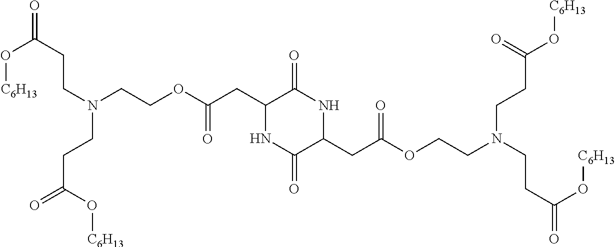 |
| 281 | 2 | C₆H₁₃ | 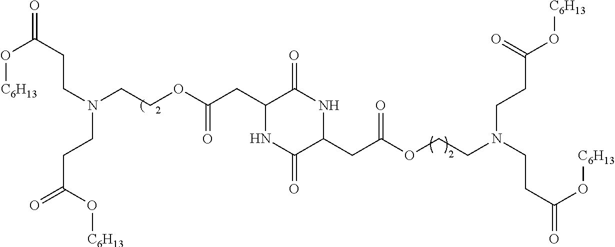 |
| 282 | 3 | C₆H₁₃ | 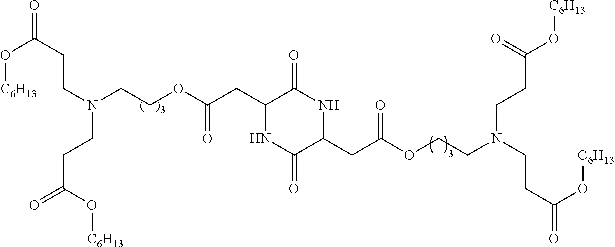 |
| 283 | 1 | C₈H₁₇ | 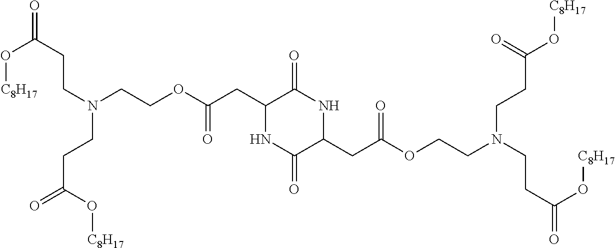 |
| 284 | 2 | C₈H₁₇ | 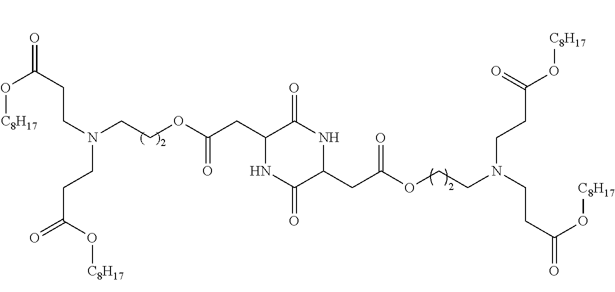 |
| 285 | 3 | C₈H₁₇ | 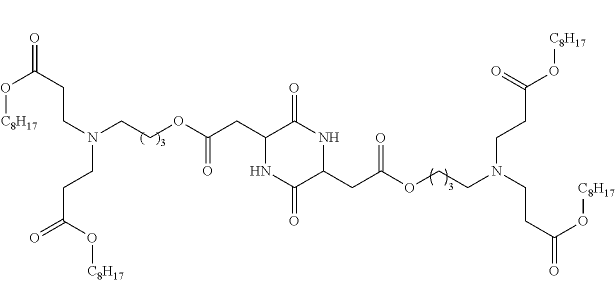 |
| 286 | 1 | C₁₀H₂₁ | 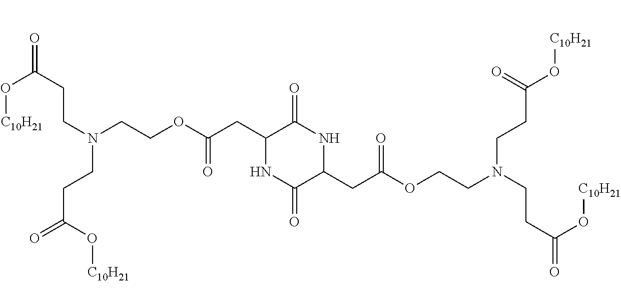 |
| 287 | 2 | C₁₀H₂₁ | 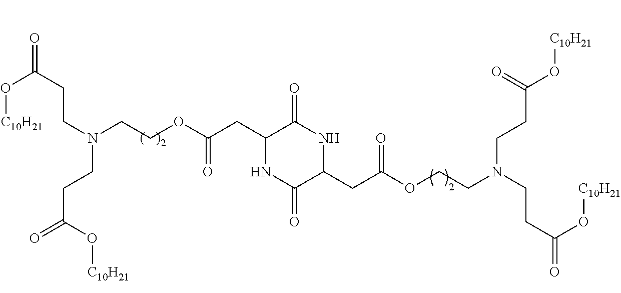 |
| 288 | 3 | C₁₀H₂₁ | 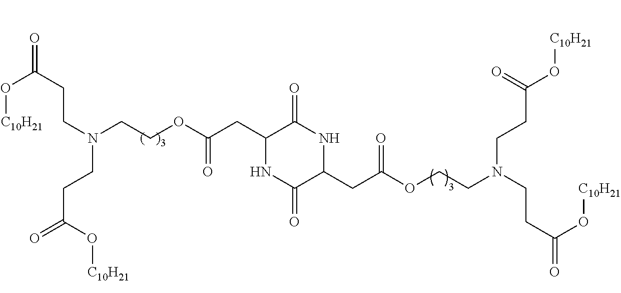 |
| 289 | 1 | C₁₂H₂₅ | 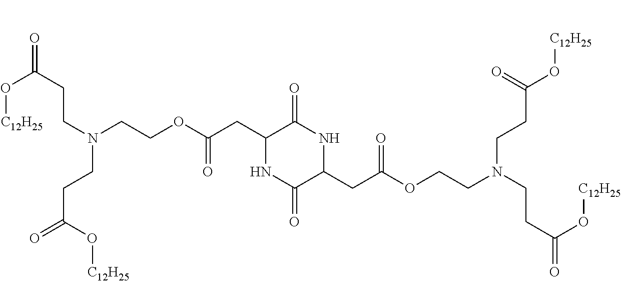 |
| 290 | 2 | C₁₂H₂₅ | 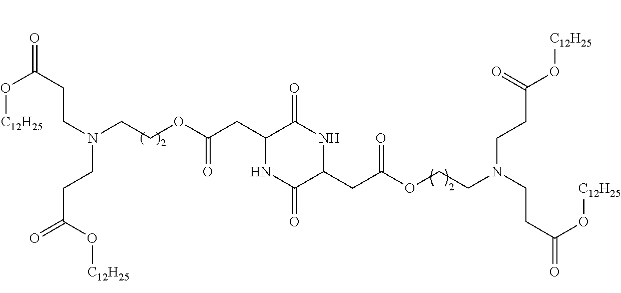 |
| 291 | 3 | C₁₂H₂₅ | 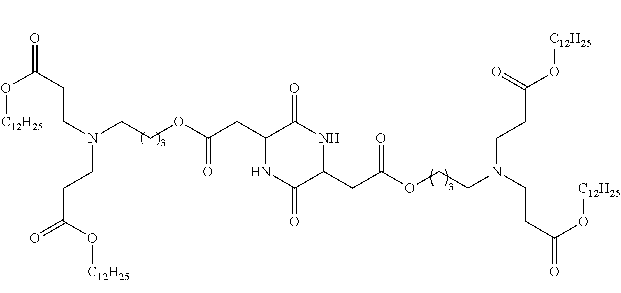 |
| 292 | 1 | C₁₄H₂₉ | 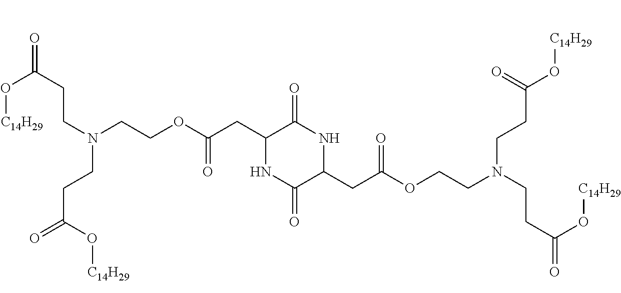 |
| 293 | 2 | C₁₄H₂₉ | 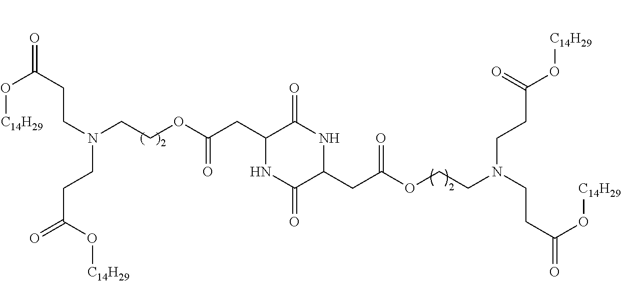 |
| 294 | 3 | C₁₄H₂₉ | 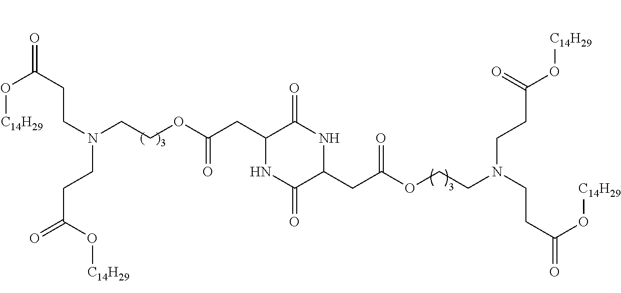 |
| 295 | 1 | C₁₆H₃₃ | 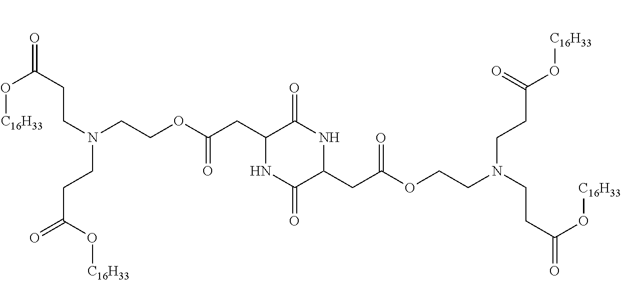 |
| 296 | 2 | C₁₆H₃₃ | 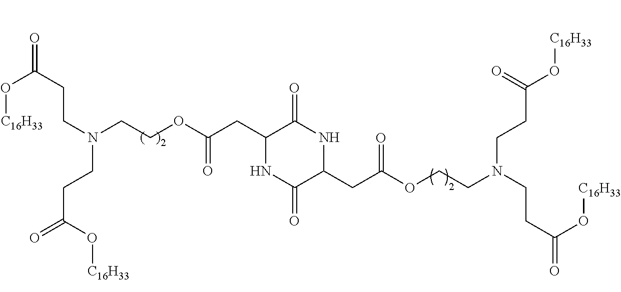 |
| 297 | 3 | C₁₆H₃₃ | 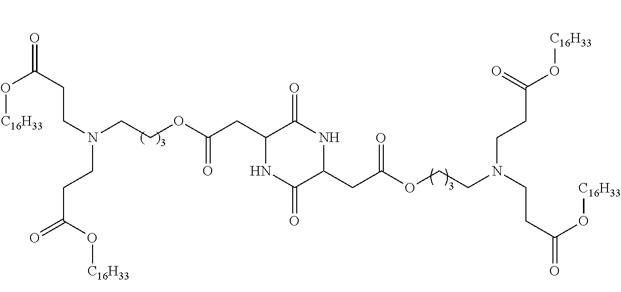 |
| 298 | 1 | C₁₈H₃₇ | 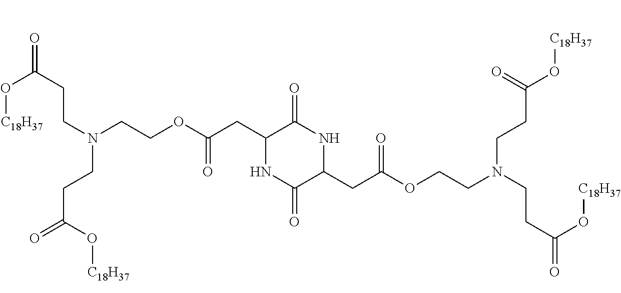 |
| 299 | 2 | C₁₈H₃₇ | 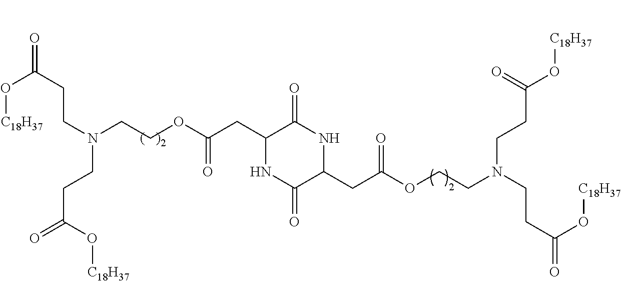 |
| 300 | 3 | C₁₈H₃₇ | 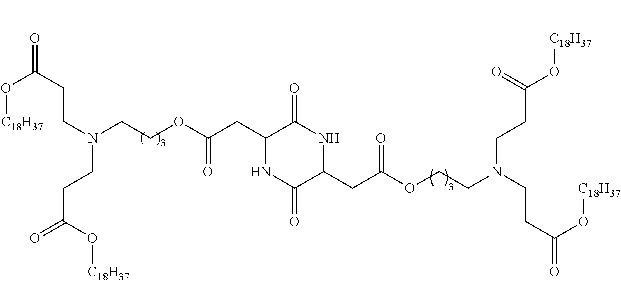 |
| 301 | 1 | C₁₈H₃₃ | 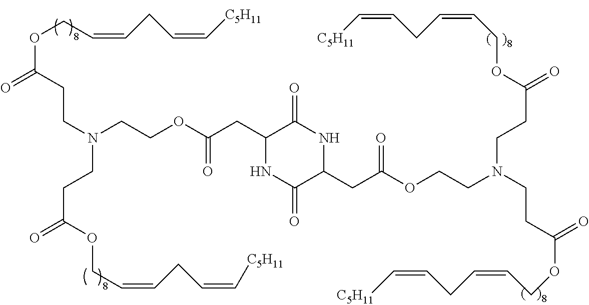 |
| 302 | 2 | C₁₈H₃₃ | 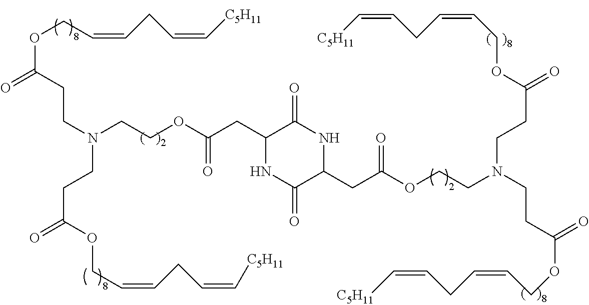 |
| 303 | 3 | C₁₈H₃₃ | 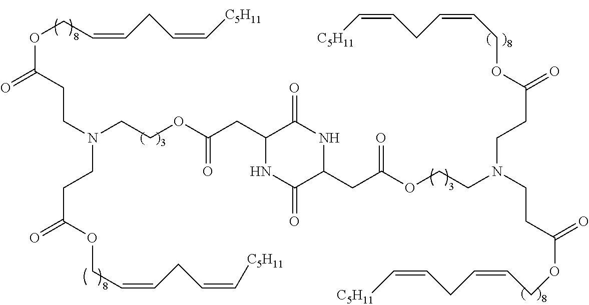 |
| 304 | 1 | C₁₈H₃₅ | 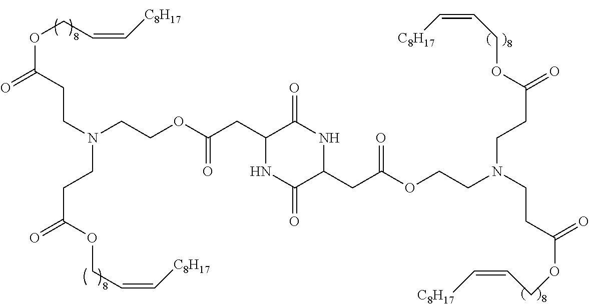 |
| 305 | 2 | C₁₈H₃₅ | 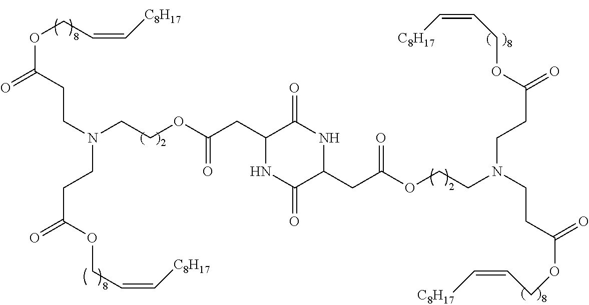 |
| 306 | 3 | C₆H₁₃ | 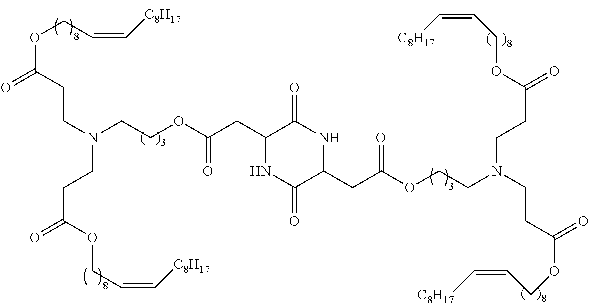 |
| 307 | 1 | C₁₈H₃₁ | 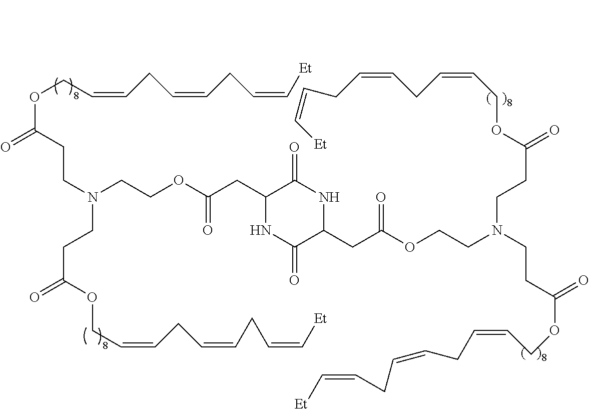 |
| 308 | 2 | C₁₈H₃₁ | 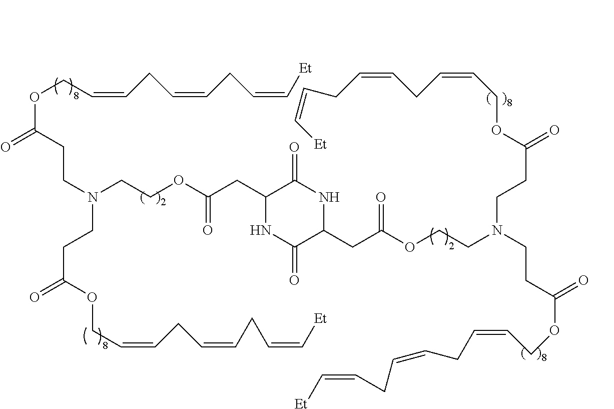 |
| 309 | 3 | C₁₈H₃₁ | 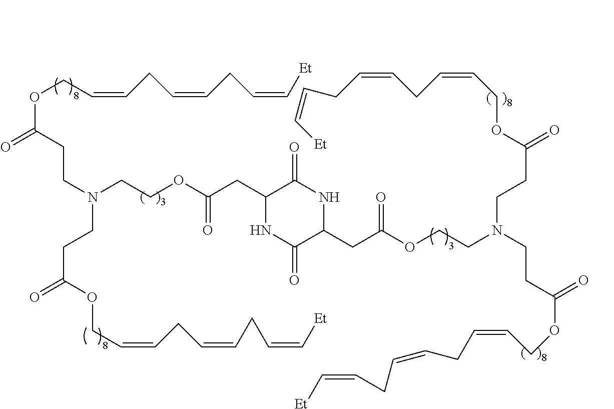 |
| 310 | 1 | substructure a | 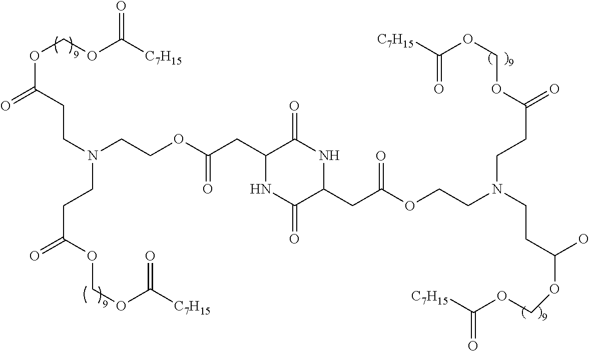 |
| 311 | 2 | substructure a | 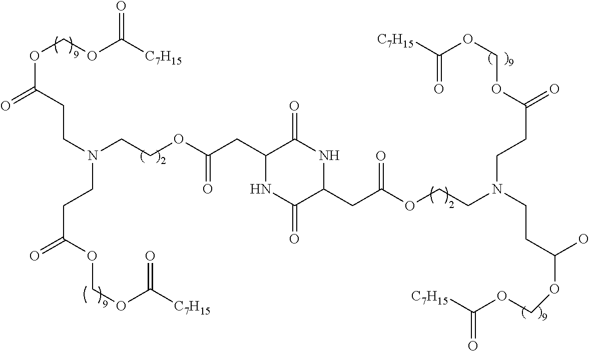 |
| 312 | 3 | substructure a | 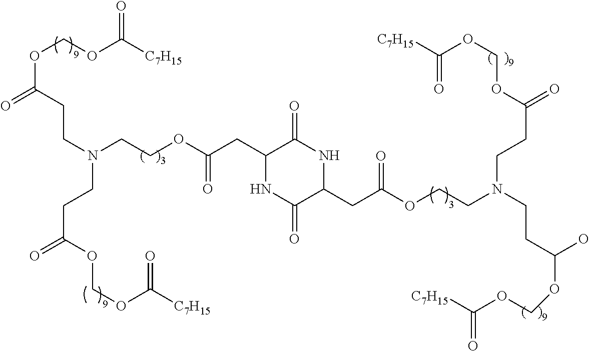 |
| 313 | 1 | substructure b | 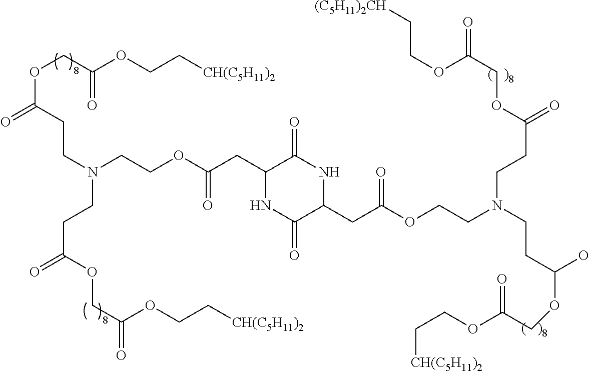 |
| 314 | 2 | substructure b | 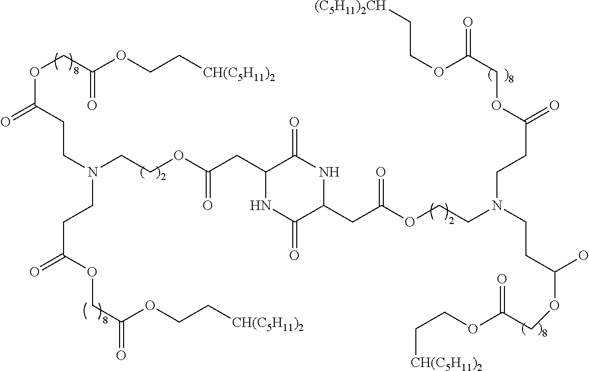 |
| 315 | 3 | substructure b | 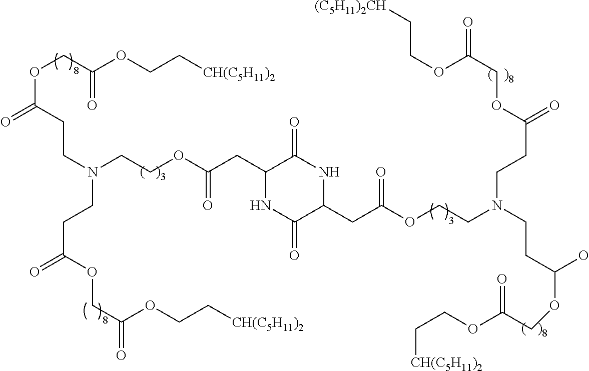 |
| 316 | 1 | —(C₇H₁₄)— CH(CH₃)₂ | 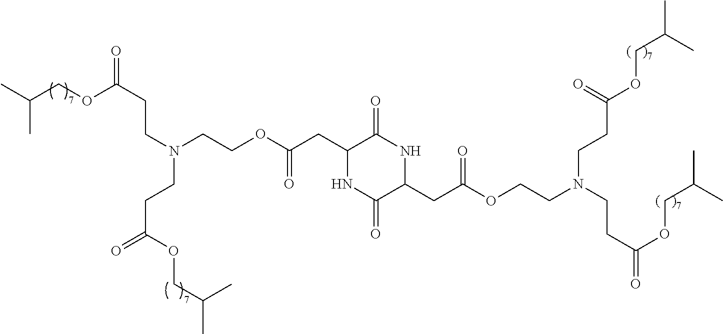 |
| 317 | 2 | —(C₇H₁₄)— CH(CH₃)₂ | 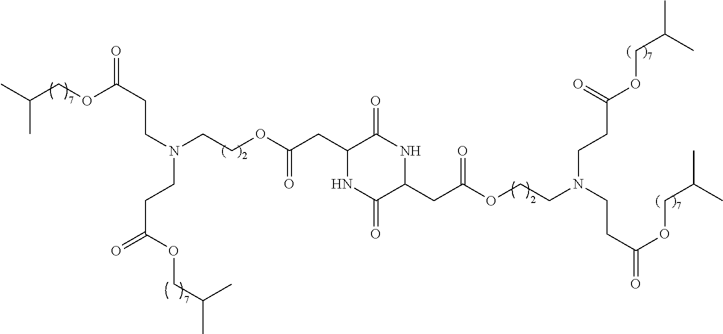 |
| 318 | 3 | —(C₇H₁₄)— CH(CH₃)₂ | 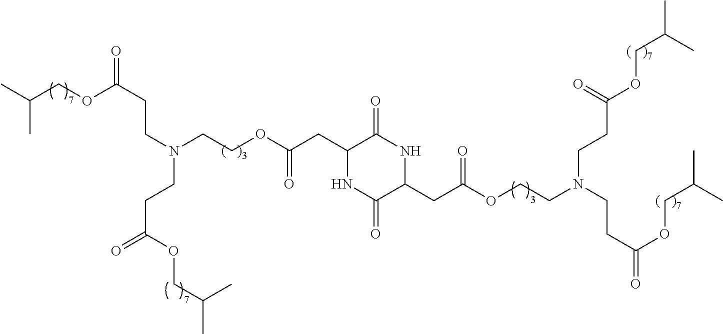 |

In embodiments, a cationic lipid is Compound 280. In embodiments, a cationic lipid is Compound 281. In embodiments, a cationic lipid is Compound 282. In embodiments, a cationic lipid is Compound 283. In embodiments, a cationic lipid is Compound 284. In embodiments, a cationic lipid is Compound 285. In embodiments, a cationic lipid is Compound 286. In embodiments, a cationic lipid is Compound 287. In embodiments, a cationic lipid is Compound 288. In embodiments, a cationic lipid is Compound 289. In embodiments, a cationic lipid is Compound 290. In embodiments, a cationic lipid is Compound 291. In embodiments, a cationic lipid is Compound 292. In embodiments, a cationic lipid is Compound 293. In embodiments, a cationic lipid is Compound 294. In embodiments, a cationic lipid is Compound 295. In embodiments, a cationic lipid is Compound 296. In embodiments, a cationic lipid is Compound 297. In embodiments, a cationic lipid is Compound 298. In embodiments, a cationic lipid is Compound 299. In embodiments, a cationic lipid is Compound 300. In embodiments, a cationic lipid is Compound 301. In embodiments, a cationic lipid is Compound 302. In embodiments, a cationic lipid is Compound 303. In embodiments, a cationic lipid is Compound 304. In embodiments, a cationic lipid is Compound 305. In embodiments, a cationic lipid is Compound 306. In embodiments, a cationic lipid is Compound 307. In embodiments, a cationic lipid is Compound 308. In embodiments, a cationic lipid is Compound 309. In embodiments, a cationic lipid is Compound 310. In embodiments, a cationic lipid is Compound 311. In embodiments, a cationic lipid is Compound 312. In embodiments, a cationic lipid is Compound 313. In embodiments, a cationic lipid is Compound 314. In embodiments, a cationic lipid is Compound 315. In embodiments, a cationic lipid is Compound 316. In embodiments, a cationic lipid is Compound 317. In embodiments, a cationic lipid is Compound 318.

Table K
cEE Thioesters- Biodegradable

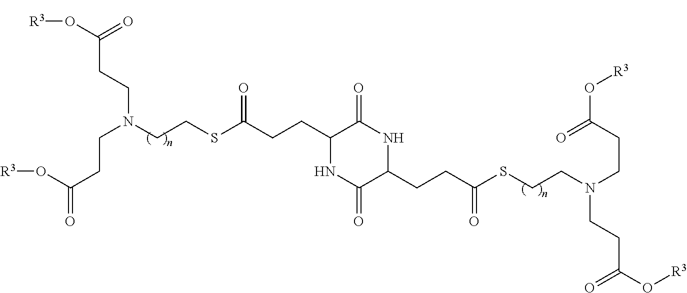

| No. | n | R³ | Structure |
|---|---|---|---|
| 319 | 1 | C₆H₁₃ | 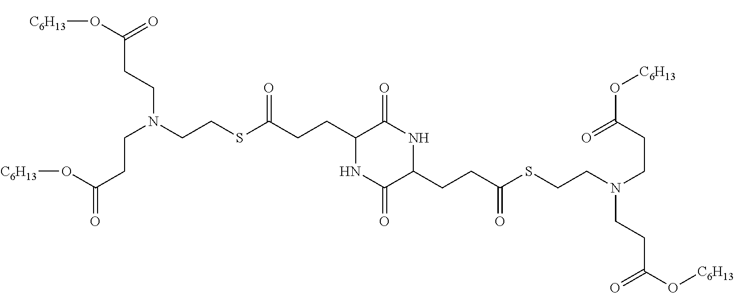 |
| 320 | 2 | C₆H₁₃ | 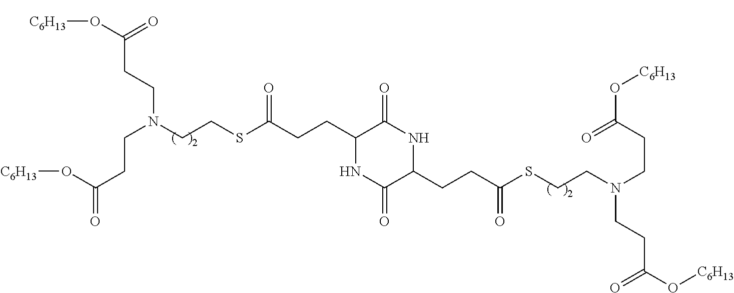 |
| 321 | 3 | C₆H₁₃ | 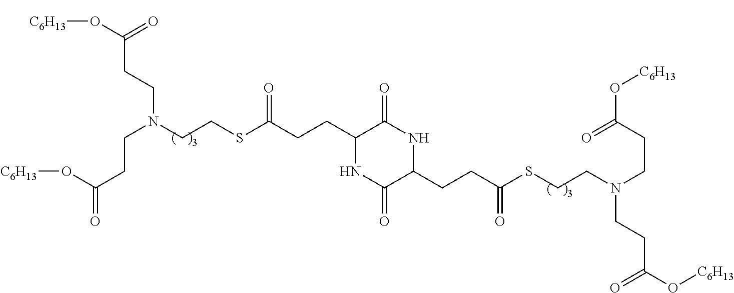 |
| 322 | 1 | C₈H₁₇ | 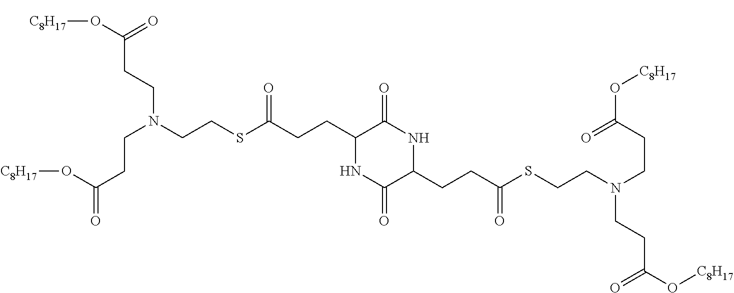 |
| 323 | 2 | C₈H₁₇ | 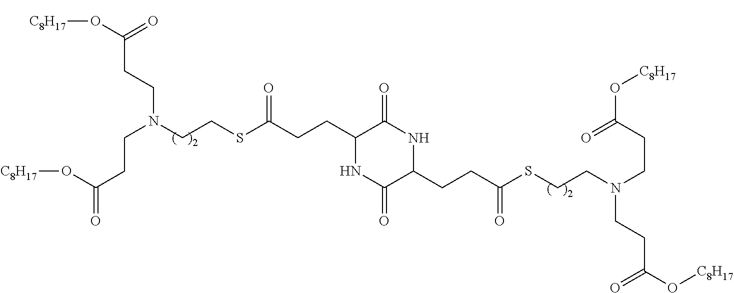 |
| 324 | 3 | C₈H₁₇ | 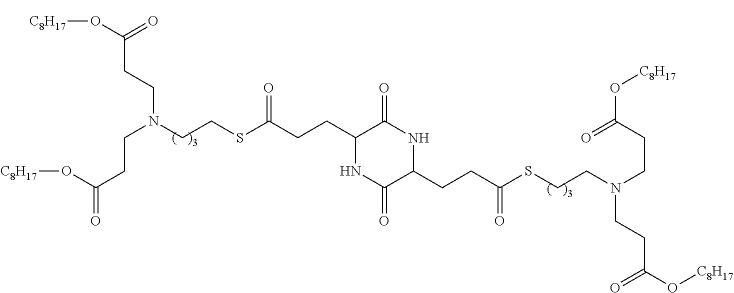 |
| 325 | 1 | C₁₀H₂₁ | 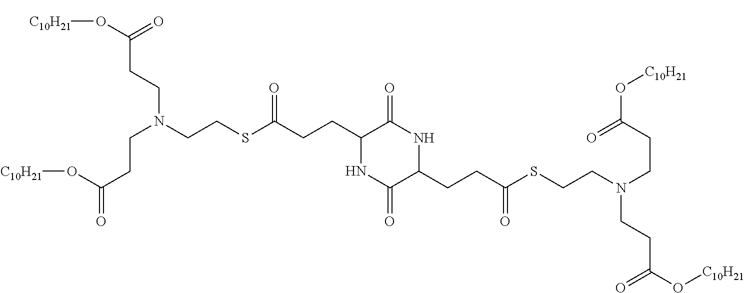 |
| 326 | 2 | C₁₀H₂₁ | 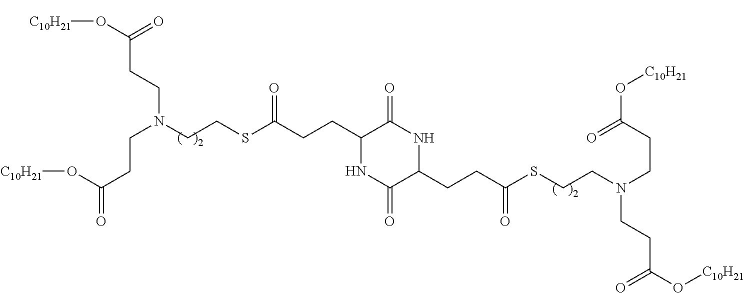 |
| 327 | 3 | C₁₀H₂₁ | 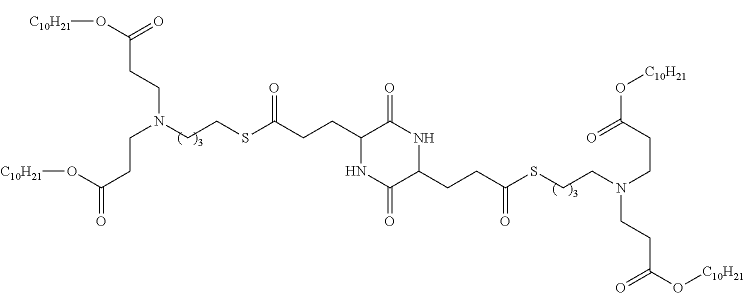 |
| 328 | 1 | C₁₂H₂₅ | 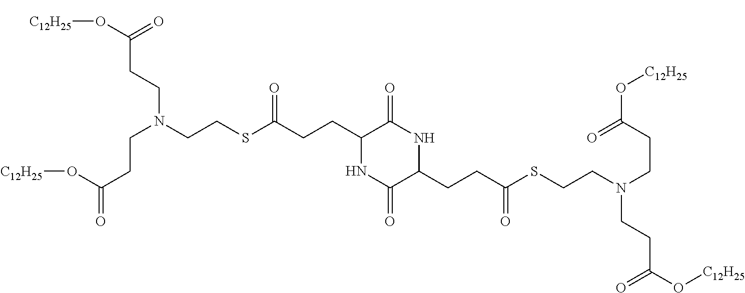 |
| 329 | 2 | C₁₂H₂₅ | 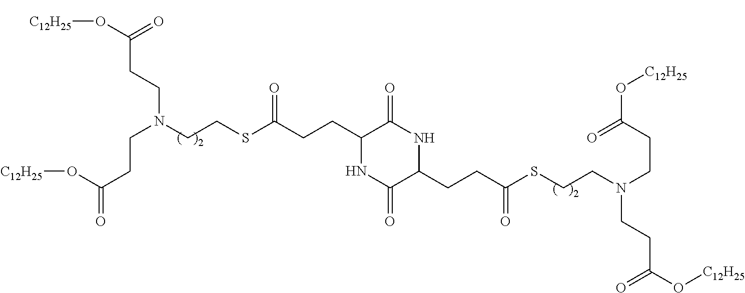 |
| 330 | 3 | C₁₂H₂₅ | 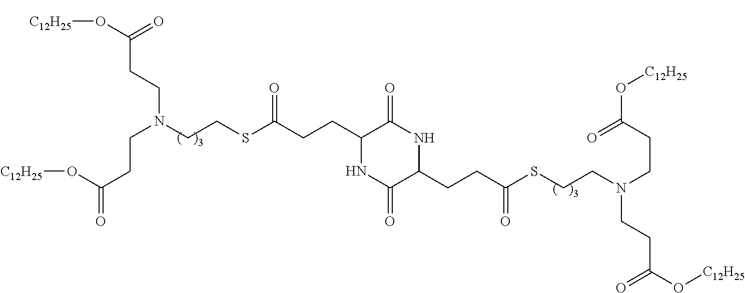 |
| 331 | 1 | C₁₄H₂₉ | 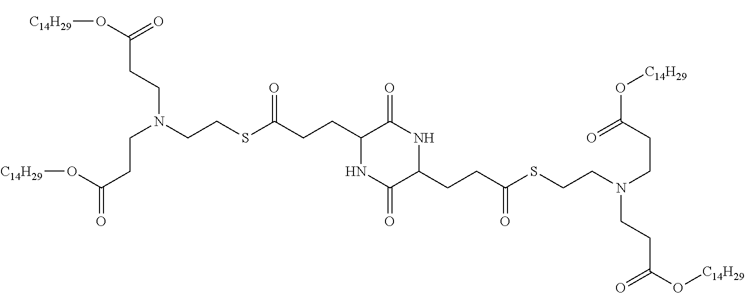 |
| 332 | 2 | C₁₄H₂₉ | 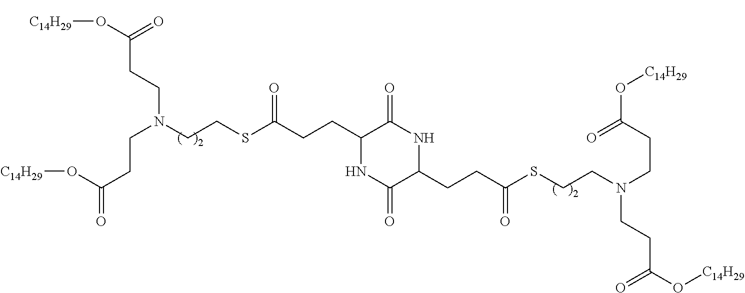 |
| 333 | 3 | C₁₄H₂₉ | 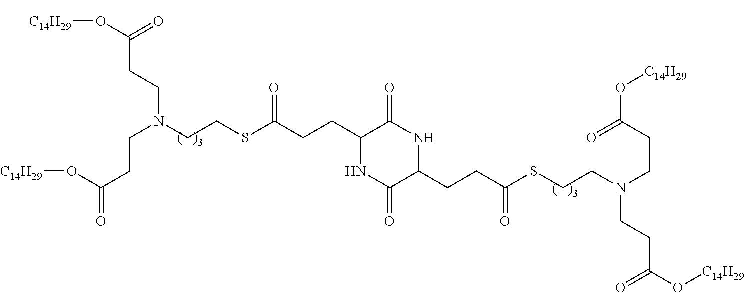 |
| 334 | 1 | C₁₆H₃₃ | 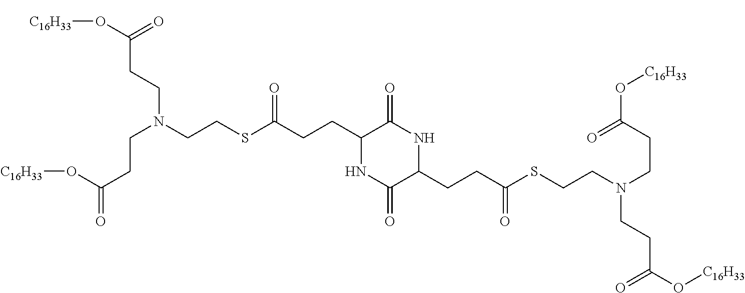 |
| 335 | 2 | C₁₆H₃₃ | 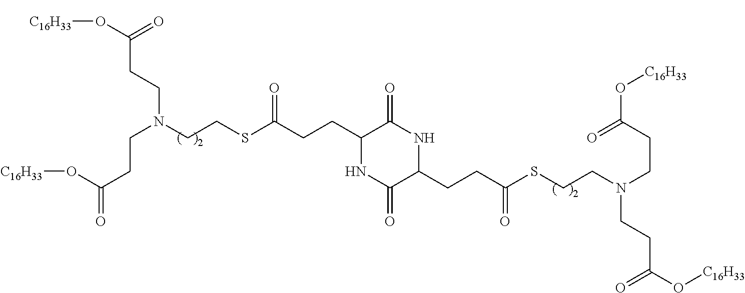 |
| 336 | 3 | C₁₆H₃₃ | 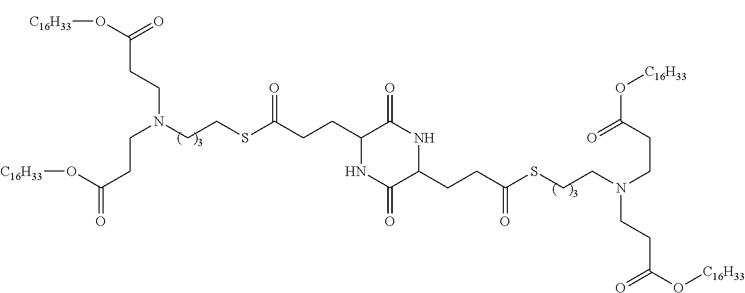 |
| 337 | 1 | C₁₈H₃₇ | 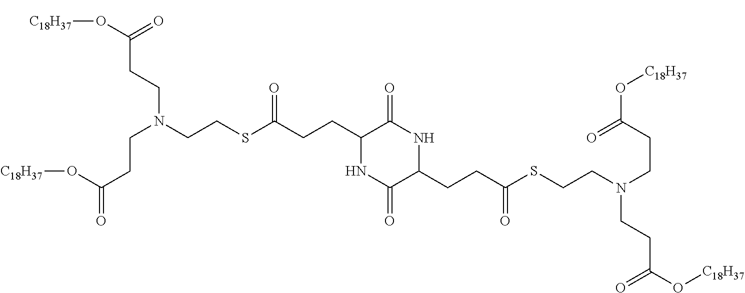 |
| 338 | 2 | C₁₈H₃₇ | 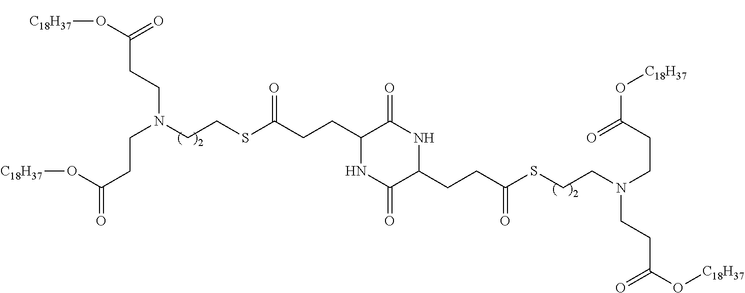 |
| 339 | 3 | C₁₈H₃₇ | 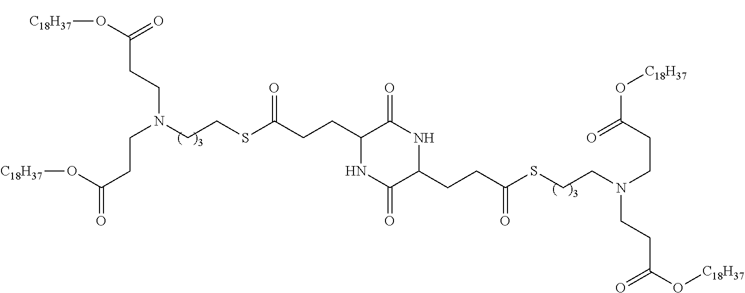 |
| 340 | 1 | C₁₈H₃₃ | 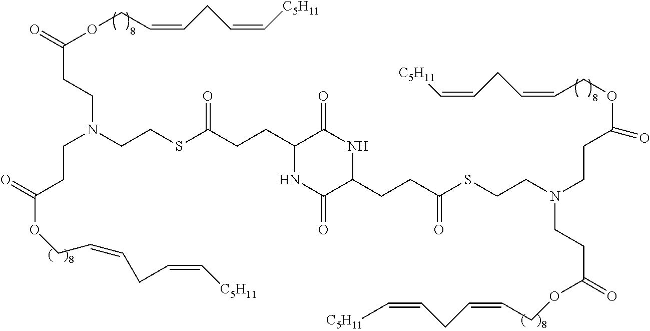 |
| 341 | 2 | C₁₈H₃₃ | 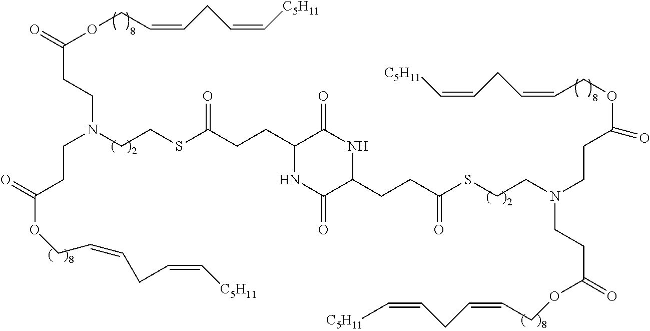 |
| 342 | 3 | C₁₈H₃₃ | 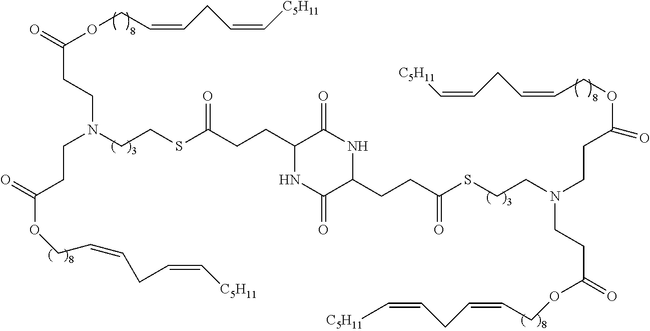 |
| 343 | 1 | C₁₈H₃₅ | 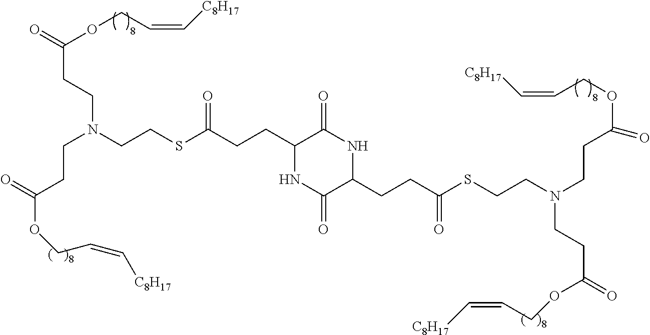 |
| 344 | 2 | C₁₈H₃₅ | 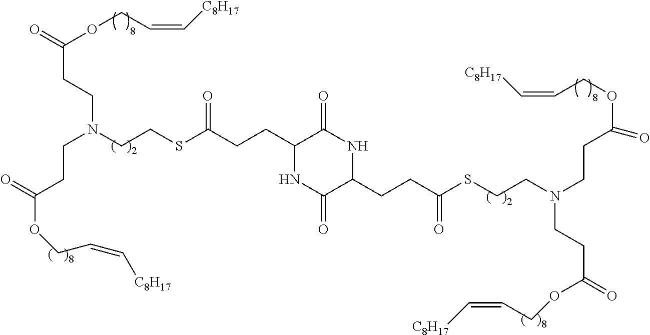 |
| 345 | 3 | C₁₈H₃₅ | 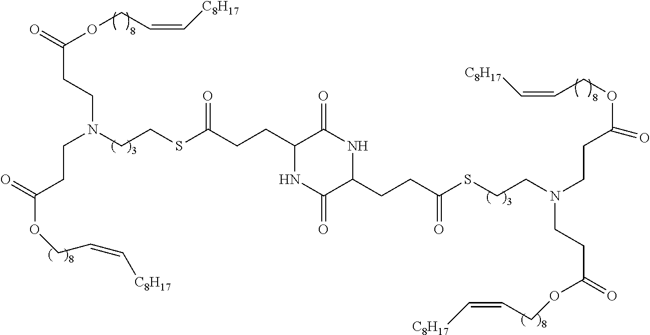 |
| 346 | 1 | C₁₈H₃₁ | 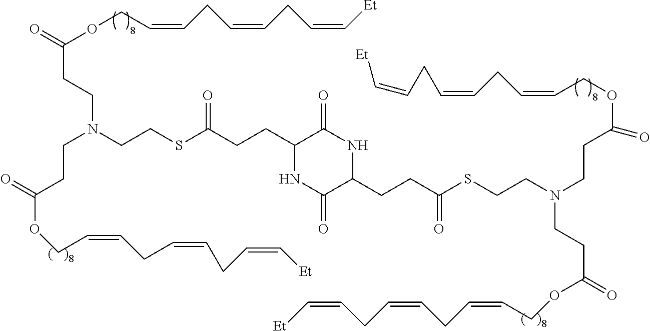 |
| 347 | 2 | C₁₈H₃₁ | 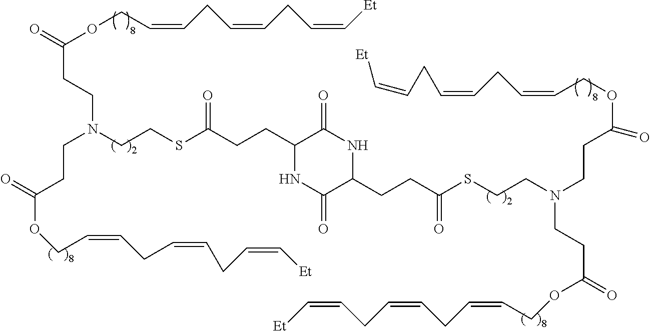 |
| 348 | 3 | C₁₈H₃₁ | 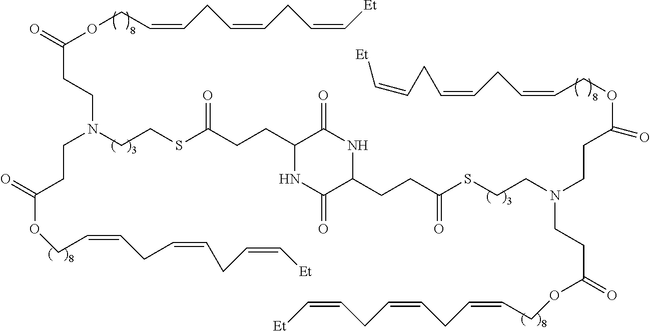 |
| 349 | 1 | substructure a | 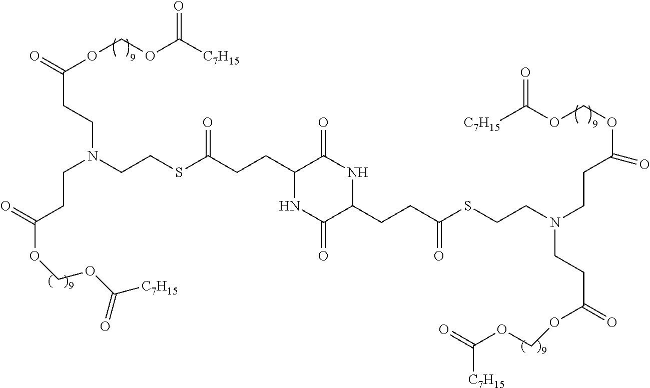 |
| 350 | 2 | substructure a | 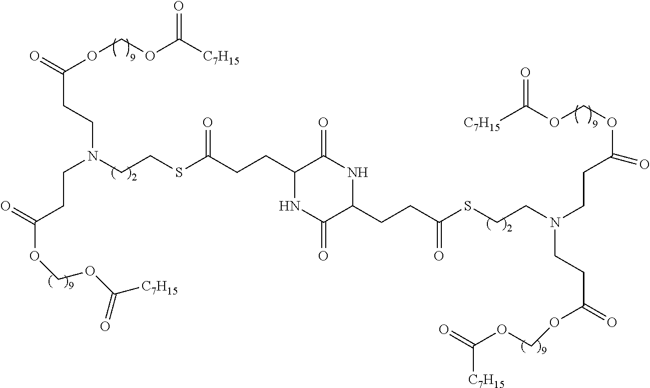 |
| 351 | 3 | substructure a | 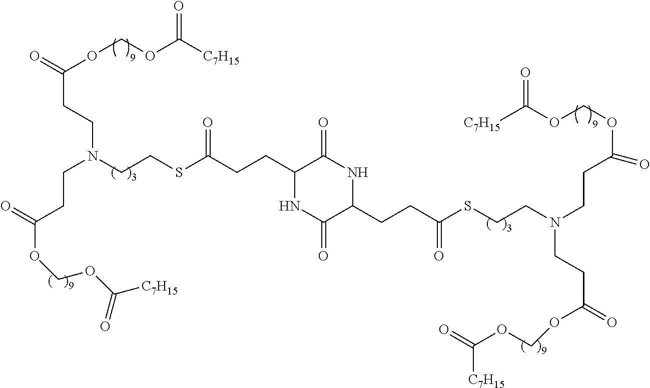 |
| 352 | 1 | substructure b | 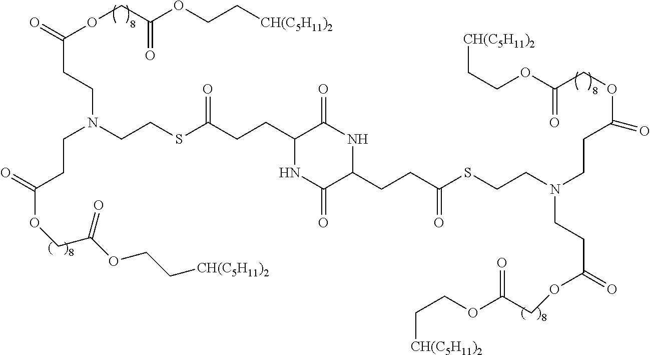 |
| 353 | 2 | substructure b | 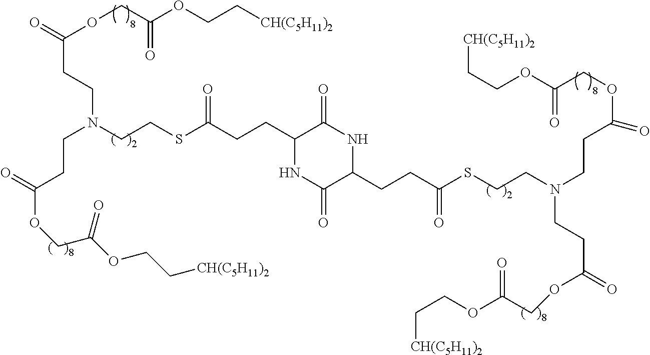 |
| 354 | 3 | substructure b | 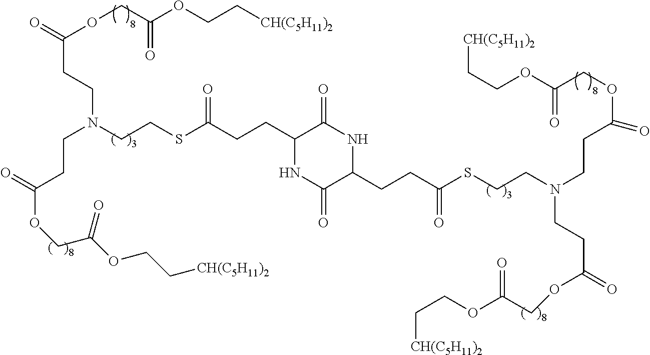 |
| 355 | 1 | —(C₇H₁₄)— CH(CH₃)₂ | 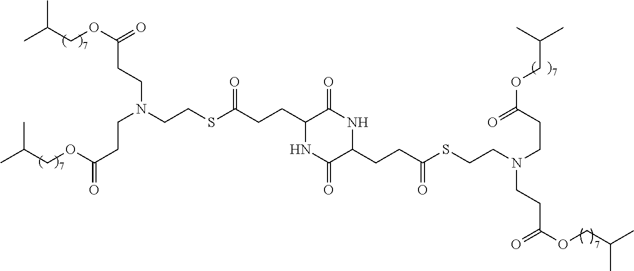 |
| 356 | 2 | —(C₇H₁₄)— CH(CH₃)₂ | 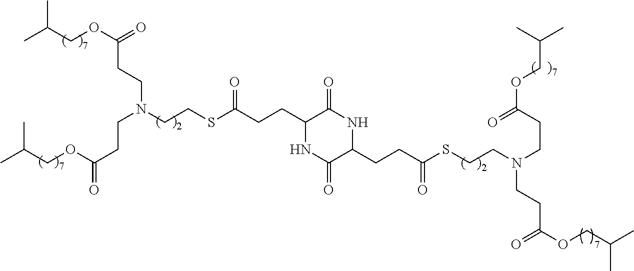 |
| 357 | 3 | —(C₇H₁₄)— CH(CH₃)₂ | 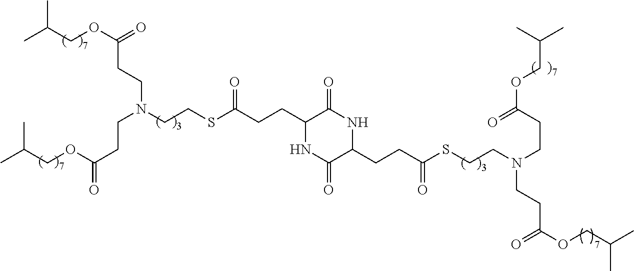 |

In embodiments, a cationic lipid is Compound 319. In embodiments, a cationic lipid is Compound 320. In embodiments, a cationic lipid is Compound 321. In embodiments, a cationic lipid is Compound 322. In embodiments, a cationic lipid is Compound 323. In embodiments, a cationic lipid is Compound 324. In embodiments, a cationic lipid is Compound 325. In embodiments, a cationic lipid is Compound 326. In embodiments, a cationic lipid is Compound 327. In embodiments, a cationic lipid is Compound 328. In embodiments, a cationic lipid is Compound 329. In embodiments, a cationic lipid is Compound 330. In embodiments, a cationic lipid is Compound 331. In embodiments, a cationic lipid is Compound 332. In embodiments, a cationic lipid is Compound 333. In embodiments, a cationic lipid is Compound 334. In embodiments, a cationic lipid is Compound 335. In embodiments, a cationic lipid is Compound 336. In embodiments, a cationic lipid is Compound 337. In embodiments, a cationic lipid is Compound 338. In embodiments, a cationic lipid is Compound 339. In embodiments, a cationic lipid is Compound 340. In embodiments, a cationic lipid is Compound 341. In embodiments, a cationic lipid is Compound 342. In embodiments, a cationic lipid is Compound 343. In embodiments, a cationic lipid is Compound 344. In embodiments, a cationic lipid is Compound 345. In embodiments, a cationic lipid is Compound 346. In embodiments, a cationic lipid is Compound 347. In embodiments, a cationic lipid is Compound 348. In embodiments, a cationic lipid is Compound 349. In embodiments, a cationic lipid is Compound 350. In embodiments, a cationic lipid is Compound 351. In embodiments, a cationic lipid is Compound 352. In embodiments, a cationic lipid is Compound 353. In embodiments, a cationic lipid is Compound 354. In embodiments, a cationic lipid is Compound 355. In embodiments, a cationic lipid is Compound 356. In embodiments, a cationic lipid is Compound 357.

TABLE L
cEE Esters-Biodegradable

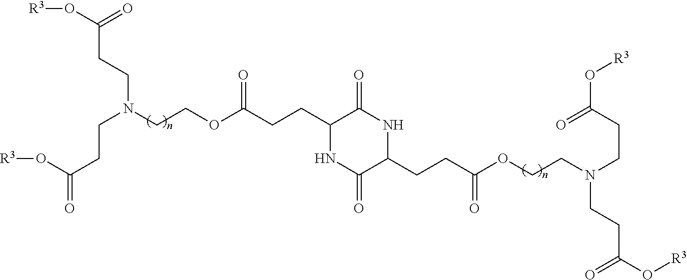

| No. | n | R³ | Structure |
|---|---|---|---|
| 358 | 1 | C₆H₁₃ | 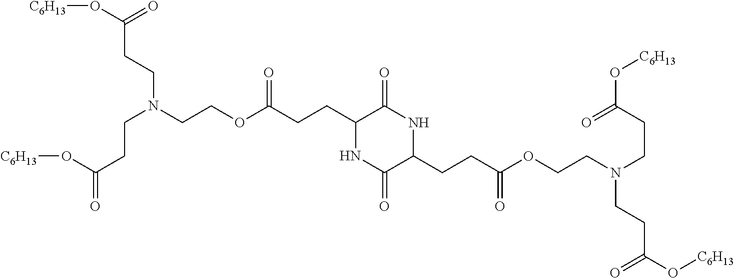 |
| 359 | 2 | C₆H₁₃ | 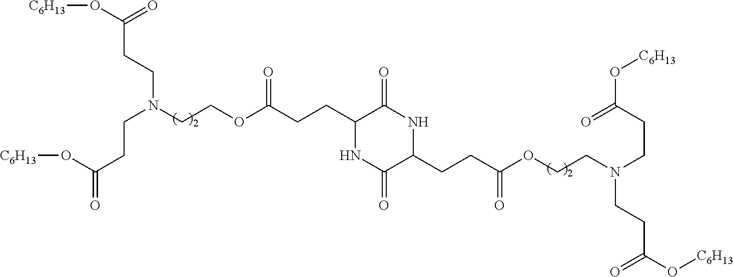 |
| 360 | 3 | C₆H₁₃ | 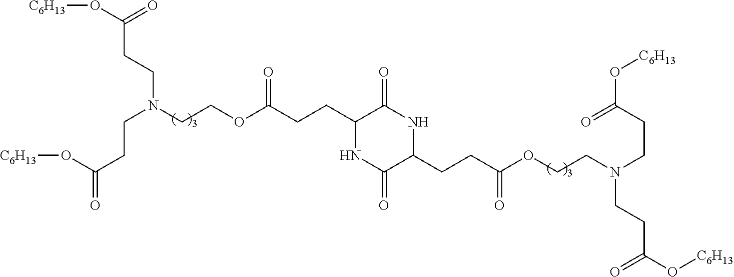 |
| 361 | 1 | C₈H₁₇ | 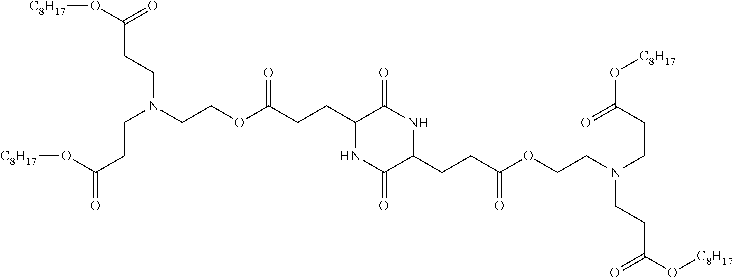 |
| 362 | 2 | C₈H₁₇ | 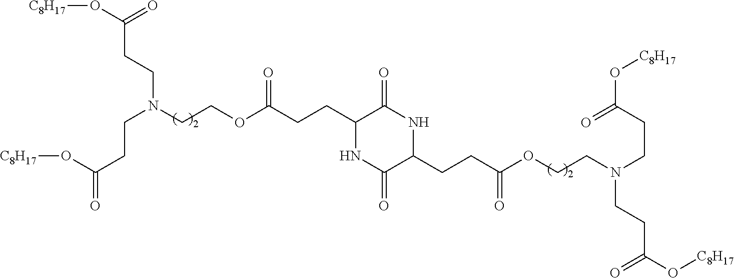 |
| 363 | 3 | C₈H₁₇ | 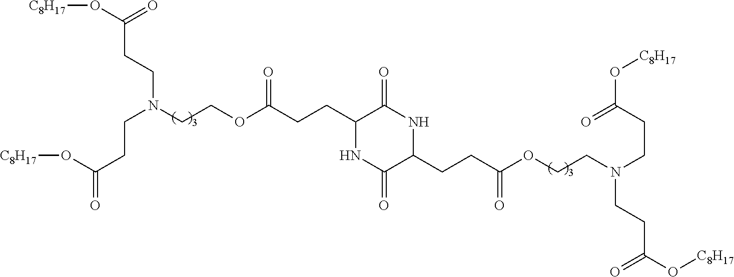 |
| 364 | 1 | C₁₀H₂₁ | 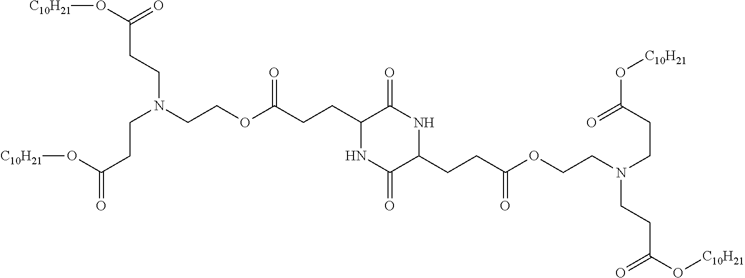 |
| 365 | 2 | C₁₀H₂₁ | 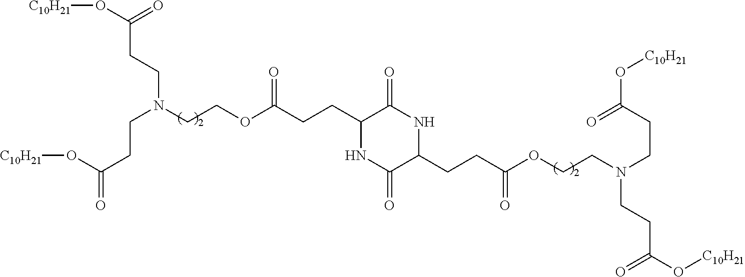 |
| 366 | 3 | C₁₀H₂₁ | 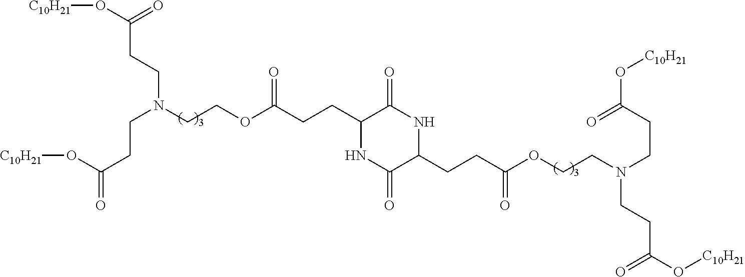 |
| 367 | 1 | C₁₂H₂₅ | 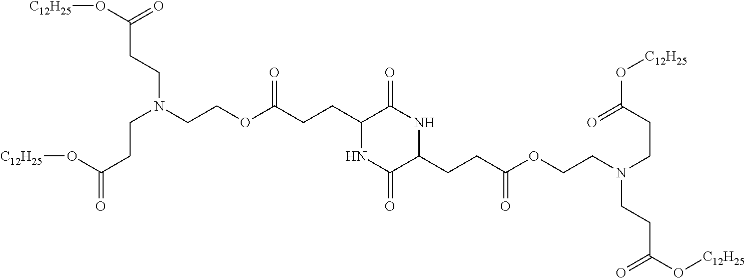 |
| 368 | 2 | C₁₂H₂₅ | 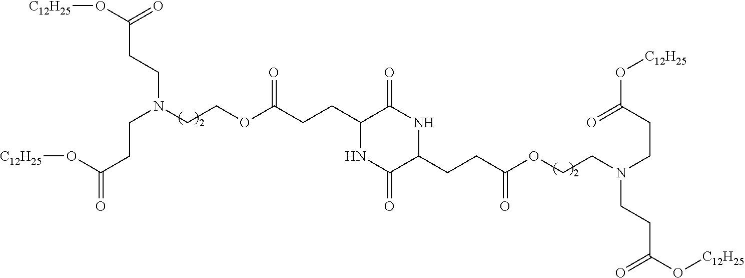 |
| 369 | 3 | C₁₂H₂₅ | 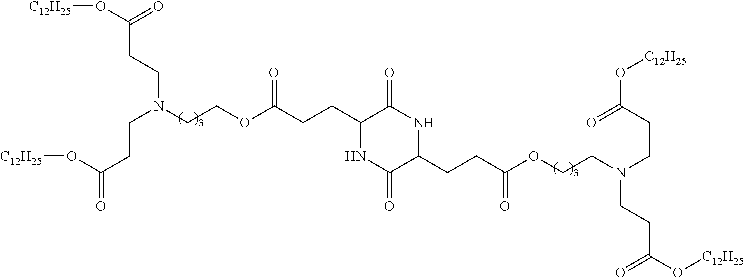 |
| 370 | 1 | C₁₄H₂₉ | 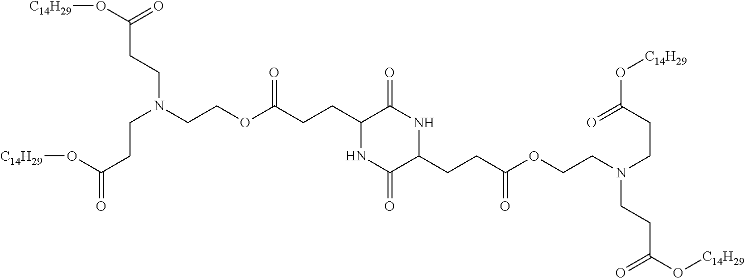 |
| 371 | 2 | C₁₄H₂₉ | 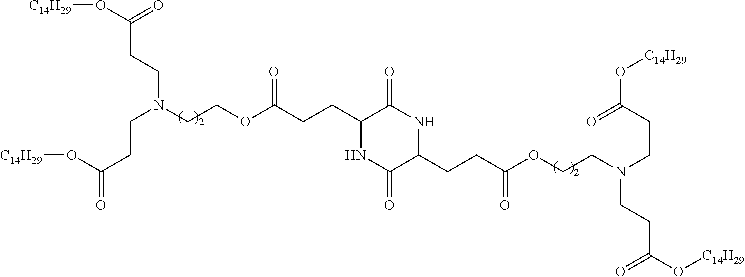 |
| 372 | 3 | C₁₄H₂₉ | 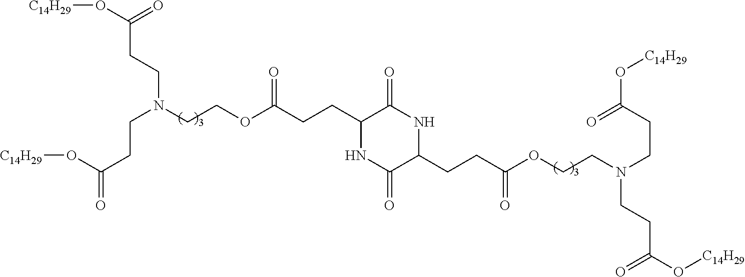 |
| 373 | 1 | C₁₆H₃₃ | 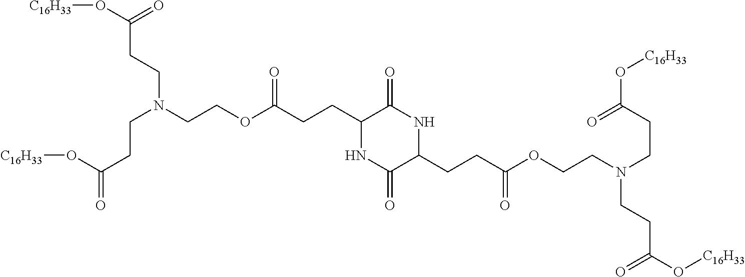 |
| 374 | 2 | C₁₆H₃₃ | 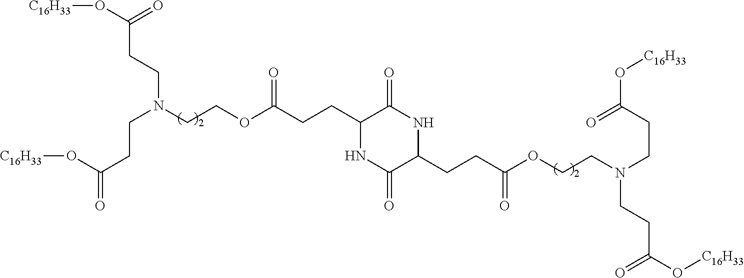 |
| 375 | 3 | C₁₆H₃₃ | 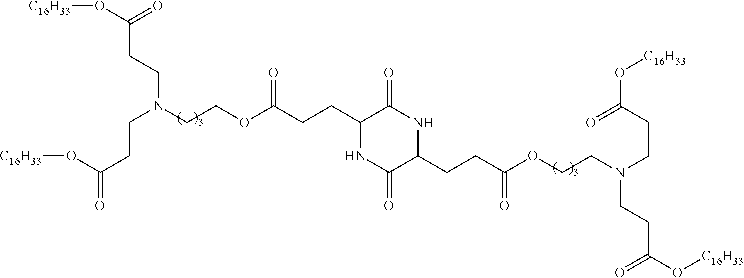 |
| 376 | 1 | C₁₈H₃₇ | 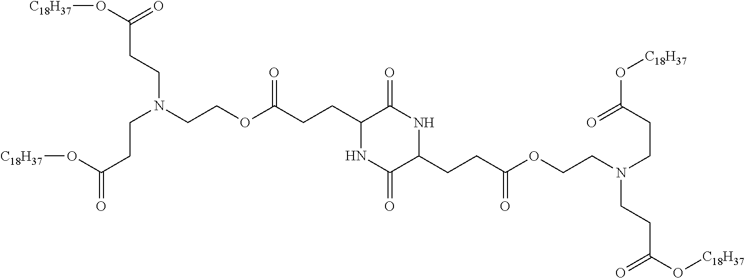 |
| 377 | 2 | C₁₈H₃₇ | 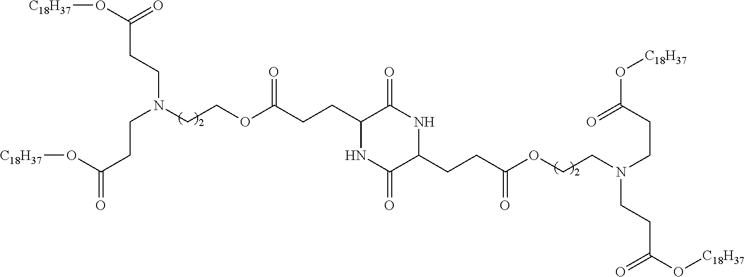 |
| 378 | 3 | C₁₈H₃₇ | 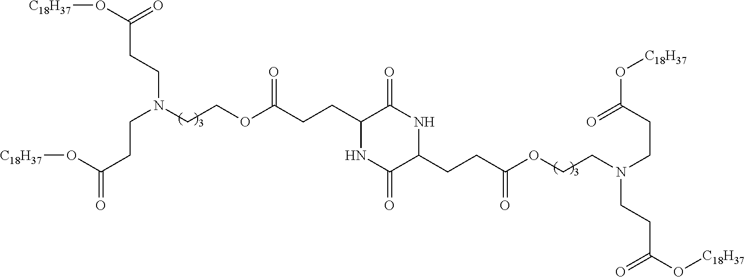 |
| 379 | 1 | C₁₈H₃₃ | 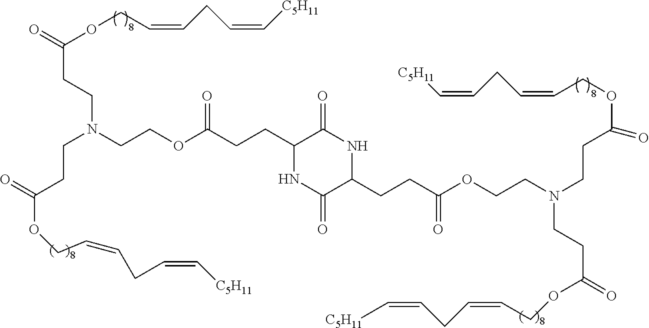 |
| 380 | 2 | C₁₈H₃₃ | 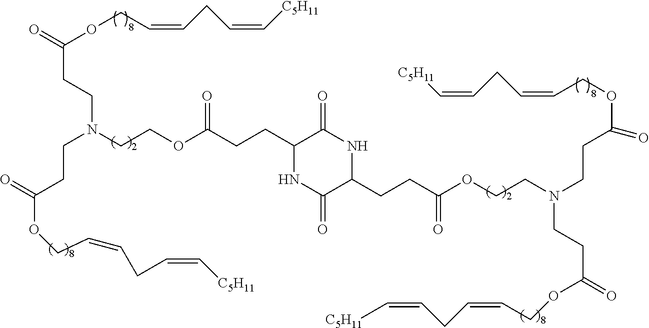 |
| 381 | 3 | C₁₈H₃₃ | 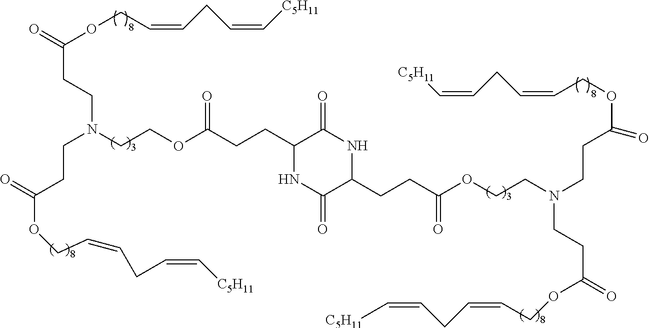 |
| 382 | 1 | C₁₈H₃₅ | 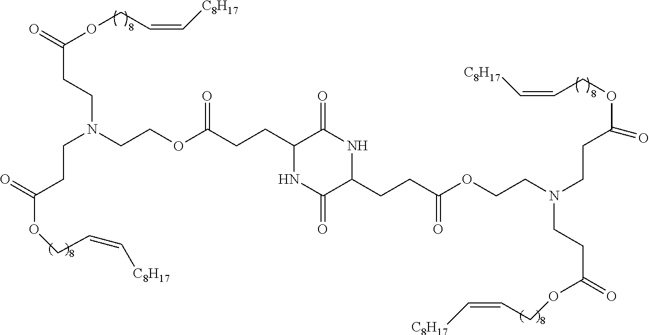 |
| 383 | 2 | C₁₈H₃₅ | 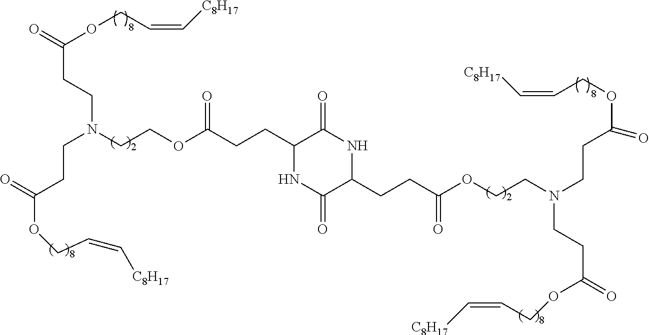 |
| 384 | 3 | C₁₈H₃₅ | 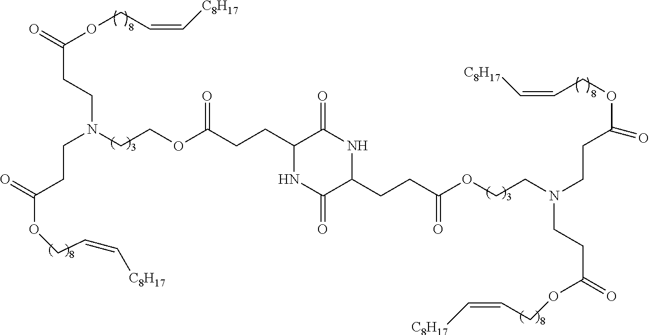 |
| 385 | 1 | C₁₈H₃₁ | 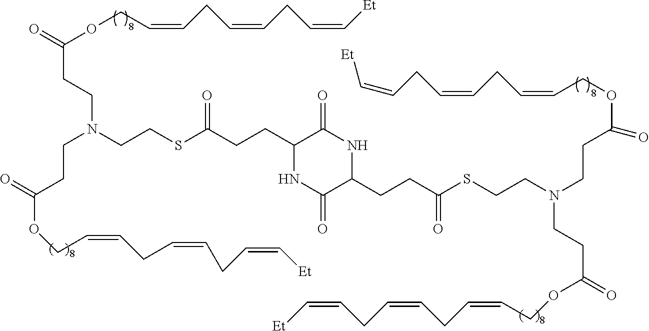 |
| 386 | 2 | C₁₈H₃₁ | 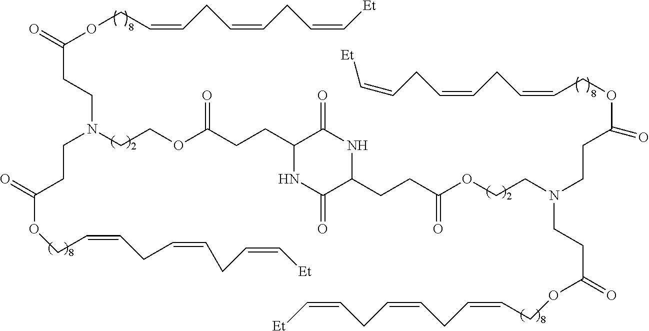 |
| 387 | 3 | C₁₈H₃₁ | 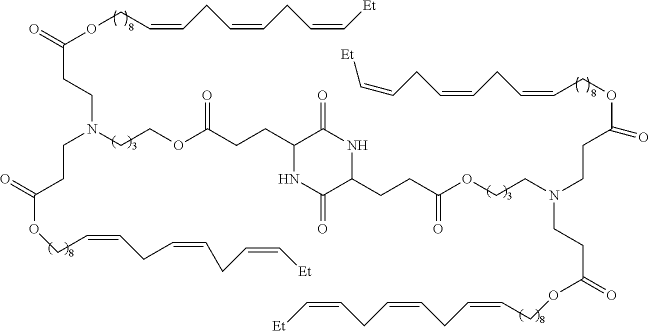 |
| 388 | 1 | substructure a | 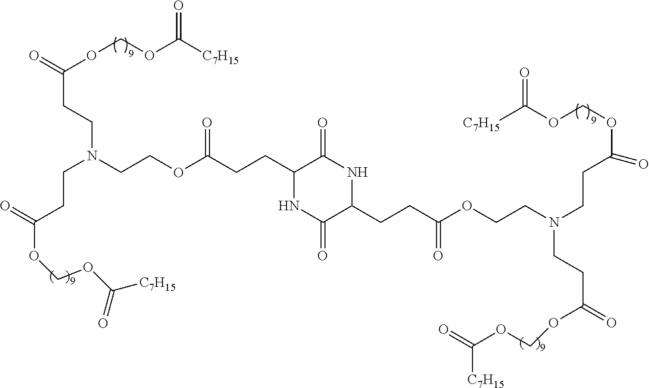 |
| 389 | 2 | substructure a | 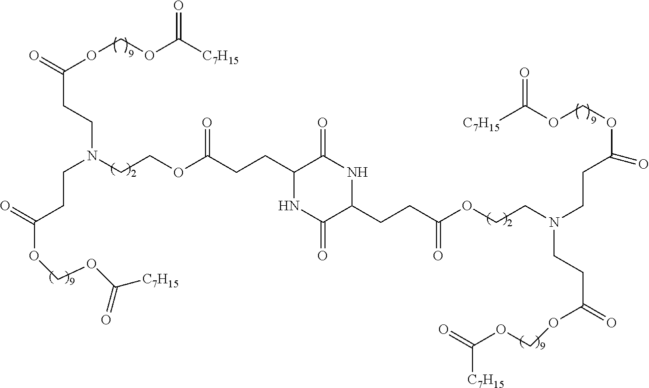 |
| 390 | 3 | substructure a | 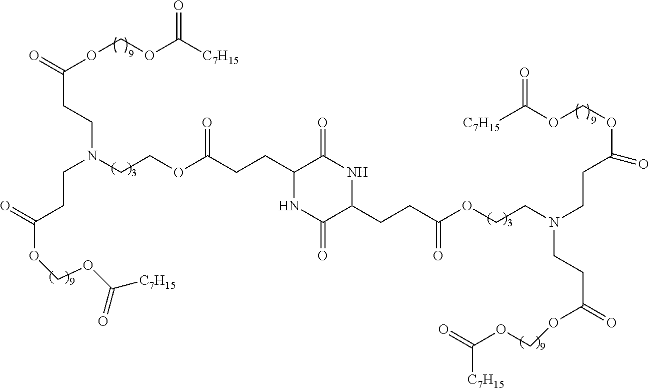 |
| 391 | 1 | substructure b | 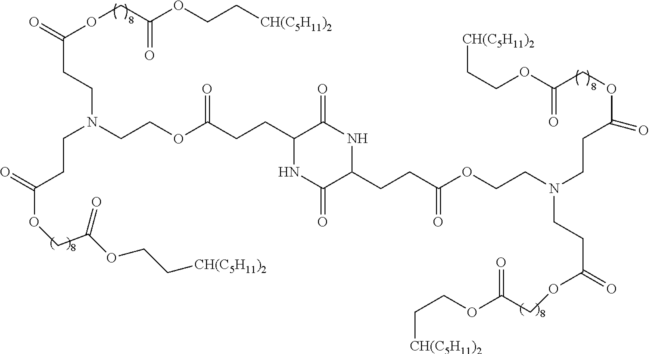 |
| 392 | 2 | substructure b | 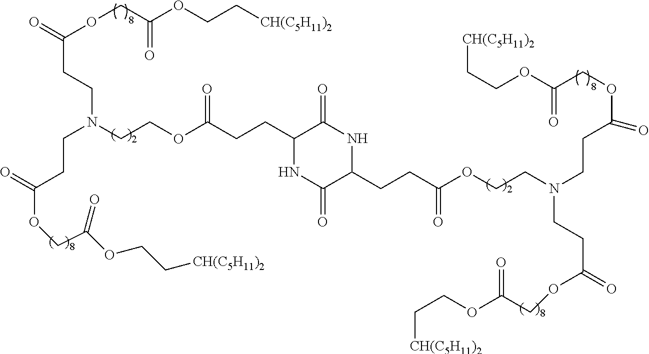 |
| 393 | 3 | substructure b | 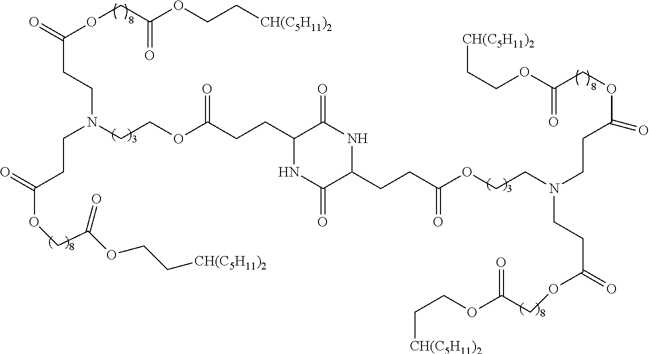 |
| 394 | 1 | —(C₇H₁₄)— CH(CH₃)₂ | 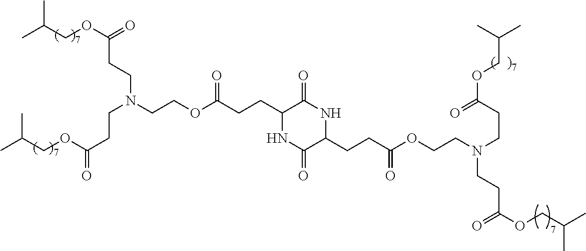 |
| 395 | 2 | —(C₇H₁₄)— CH(CH₃)₂ | 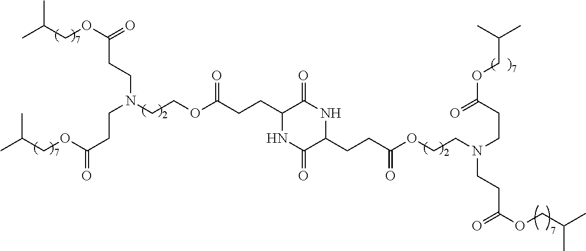 |
| 396 | 3 | —(C₇H₁₄)— CH(CH₃)₂ | 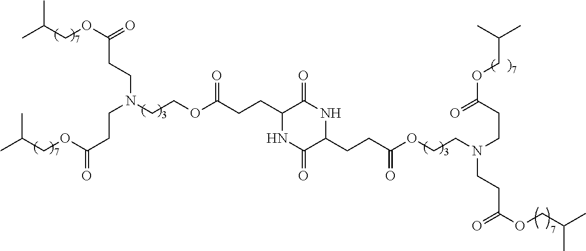 |

In embodiments, a cationic lipid is Compound 358. In embodiments, a cationic lipid is Compound 359. In embodiments, a cationic lipid is Compound 360. In embodiments, a cationic lipid is Compound 361. In embodiments, a cationic lipid is Compound 362. In embodiments, a cationic lipid is Compound 363. In embodiments, a cationic lipid is Compound 364. In embodiments, a cationic lipid is Compound 365. In embodiments, a cationic lipid is Compound 366. In embodiments, a cationic lipid is Compound 367. In embodiments, a cationic lipid is Compound 368. In embodiments, a cationic lipid is Compound 369. In embodiments, a cationic lipid is Compound 370. In embodiments, a cationic lipid is Compound 371. In embodiments, a cationic lipid is Compound 372. In embodiments, a cationic lipid is Compound 373. In embodiments, a cationic lipid is Compound 374. In embodiments, a cationic lipid is Compound 375. In embodiments, a cationic lipid is Compound 376. In embodiments, a cationic lipid is Compound 377. In embodiments, a cationic lipid is Compound 378. In embodiments, a cationic lipid is Compound 379. In embodiments, a cationic lipid is Compound 380. In embodiments, a cationic lipid is Compound 381. In embodiments, a cationic lipid is Compound 382. In embodiments, a cationic lipid is Compound 383. In embodiments, a cationic lipid is Compound 384. In embodiments, a cationic lipid is Compound 385. In embodiments, a cationic lipid is Compound 386. In embodiments, a cationic lipid is Compound 387. In embodiments, a cationic lipid is Compound 388. In embodiments, a cationic lipid is Compound 389. In embodiments, a cationic lipid is Compound 390. In embodiments, a cationic lipid is Compound 391. In embodiments, a cationic lipid is Compound 392. In embodiments, a cationic lipid is Compound 393. In embodiments, a cationic lipid is Compound 394. In embodiments, a cationic lipid is Compound 395. In embodiments, a cationic lipid is Compound 396.

TABLE M
Homoserine (cHse) Lipids-Biodegradable

| | |
|---|---|
| | (III-f) |
| 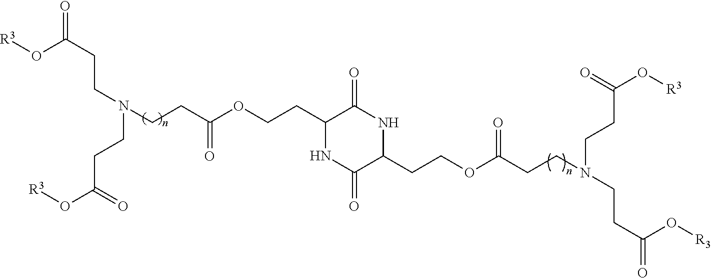 | |

| No. | n | R³ | Structure |
|---|---|---|---|
| 397 | 1 | C₆H₁₃ | 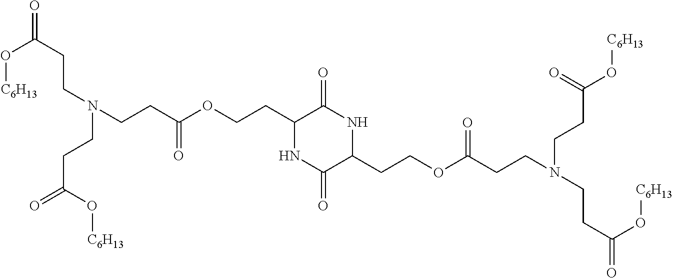 |
| 398 | 2 | C₆H₁₃ | 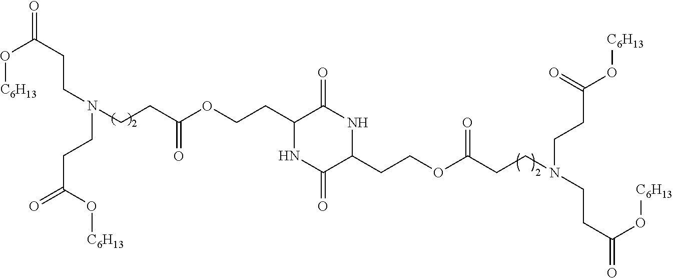 |
| 399 | 3 | C₆H₁₃ | 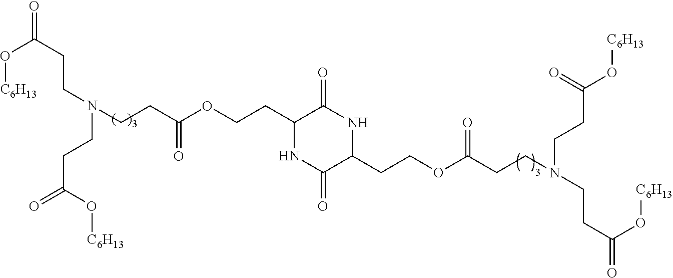 |
| 400 | 1 | C₈H₁₇ | 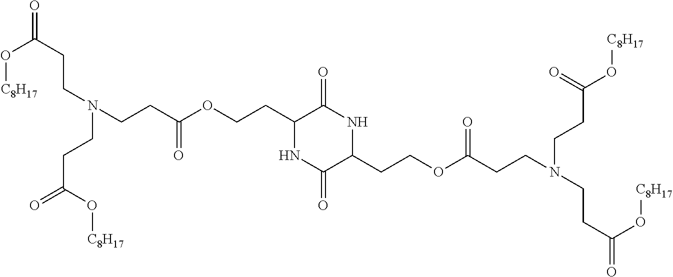 |
| 401 | 2 | C₈H₁₇ | 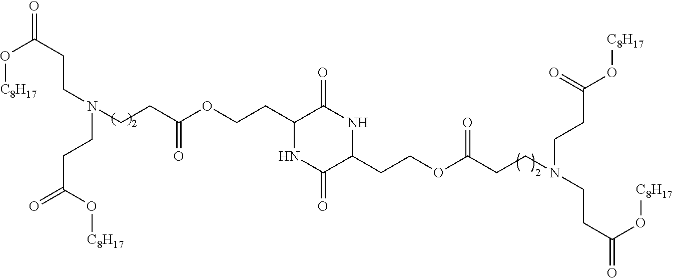 |
| 402 | 3 | C₈H₁₇ | 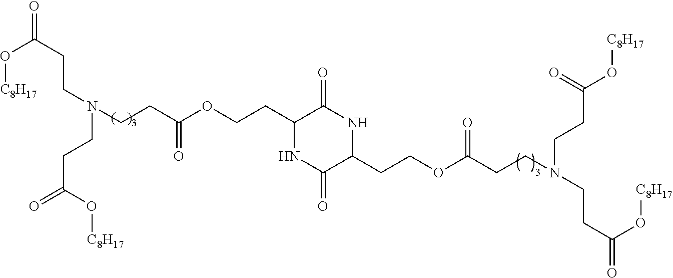 |
| 403 | 1 | C₁₀H₂₁ | 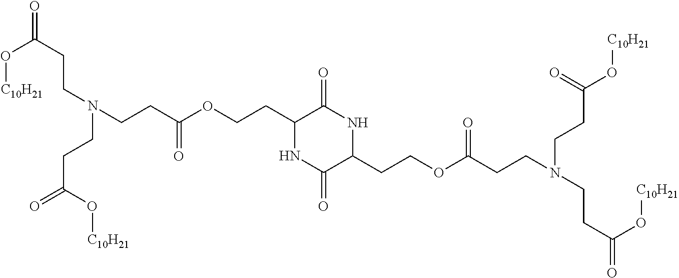 |
| 404 | 2 | C₁₀H₂₁ | 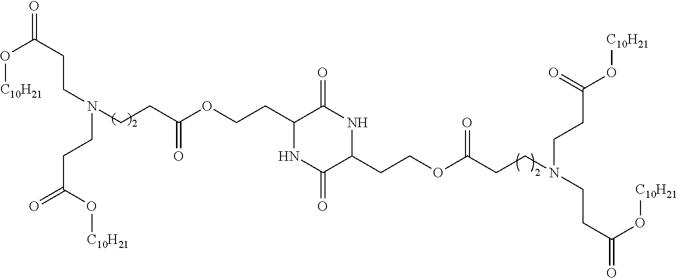 |
| 405 | 3 | C₁₀H₂₁ | 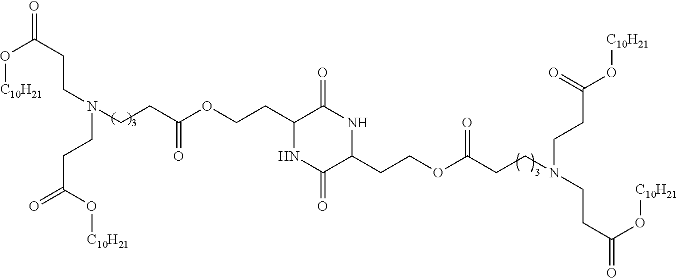 |
| 406 | 1 | C₁₂H₂₅ | 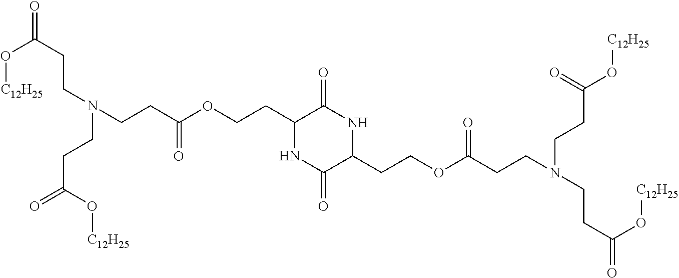 |
| 407 | 2 | C₁₂H₂₅ | 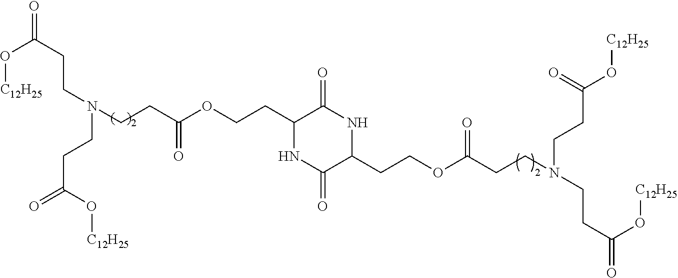 |
| 408 | 3 | C₁₂H₂₅ | 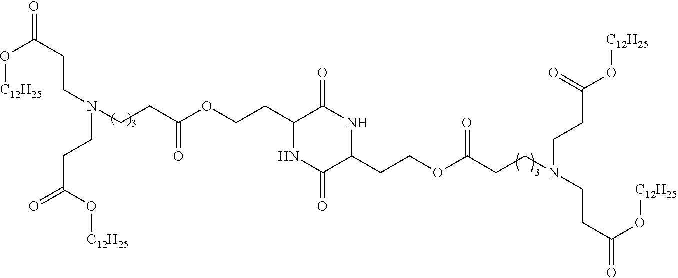 |
| 409 | 1 | C₁₄H₂₉ | 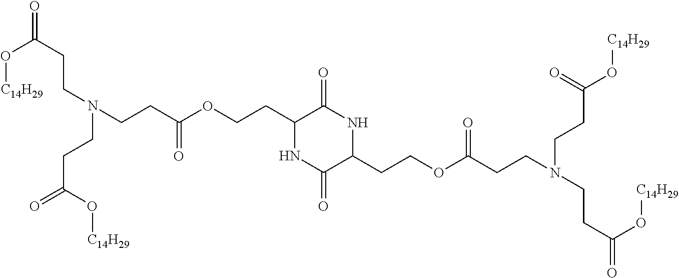 |
| 410 | 2 | C₁₄H₂₉ | 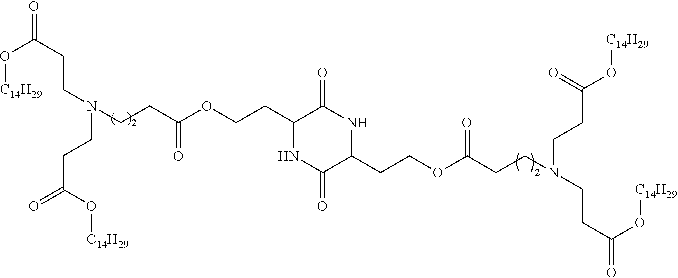 |
| 411 | 3 | C₁₄H₂₉ | 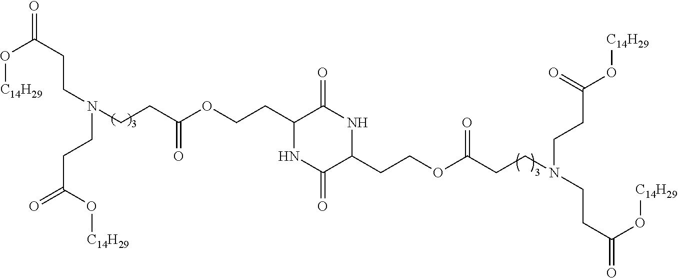 |
| 412 | 1 | C₁₆H₃₃ | 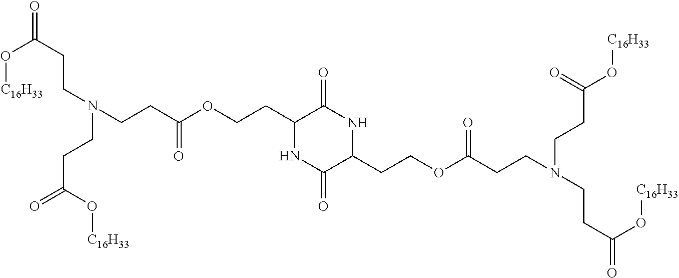 |
| 413 | 2 | C₁₆H₃₃ | 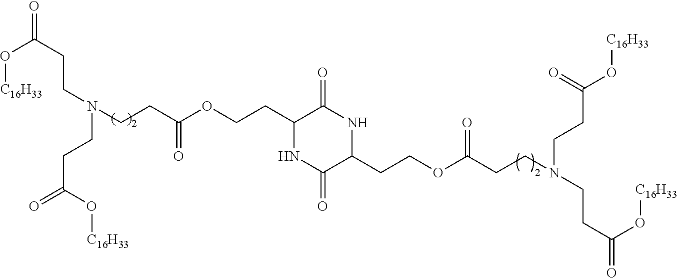 |
| 414 | 3 | C₁₆H₃₃ | 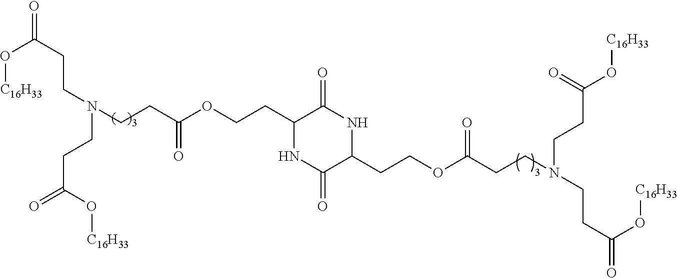 |
| 415 | 1 | C₁₈H₃₇ | 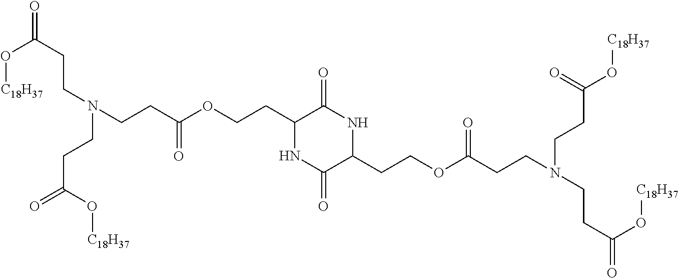 |
| 416 | 2 | C₁₈H₃₇ | 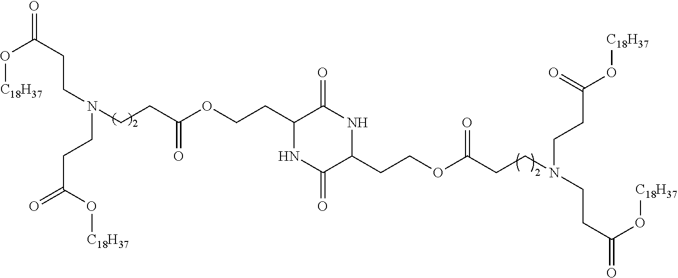 |
| 417 | 3 | C₁₈H₃₇ | 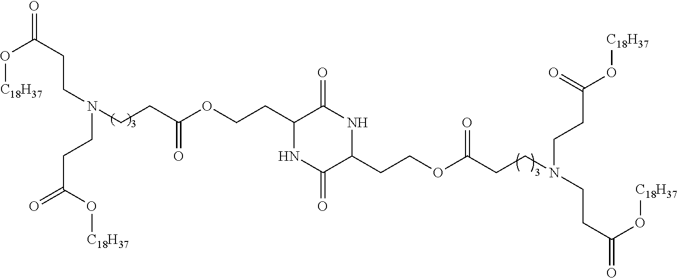 |
| 418 | 1 | C₁₈H₃₃ | 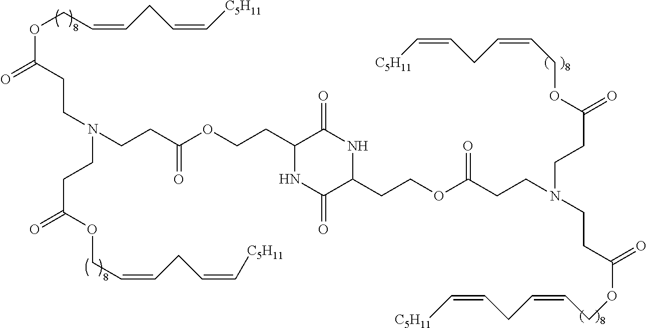 |
| 419 | 2 | C₁₈H₃₃ | 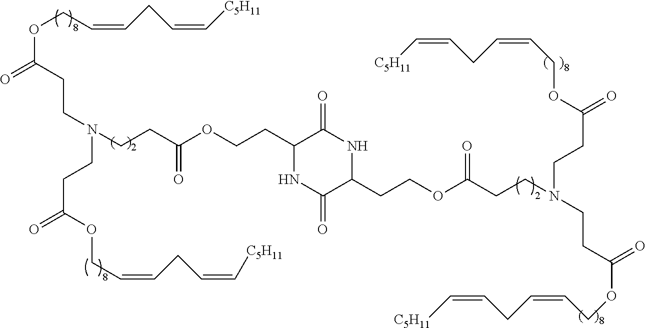 |
| 420 | 3 | C₁₈H₃₃ | 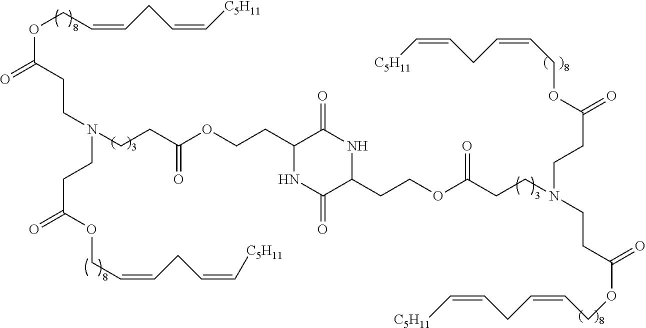 |
| 421 | 1 | C₁₈H₃₅ | 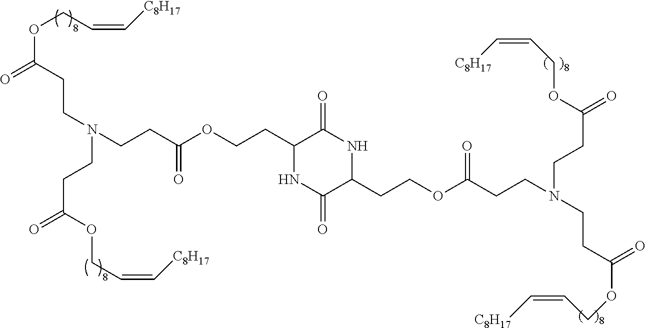 |
| 422 | 2 | C₁₈H₃₅ | 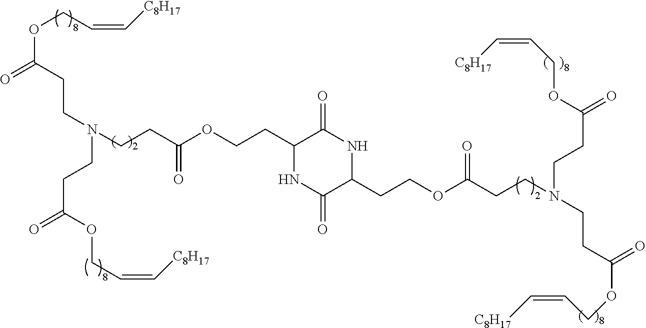 |
| 423 | 3 | C₁₈H₃₅ | 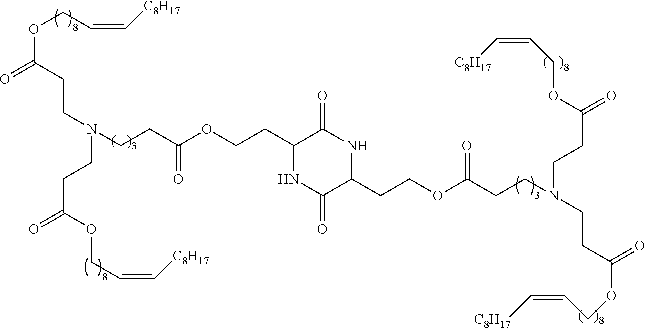 |
| 424 | 1 | C₁₈H₃₁ | 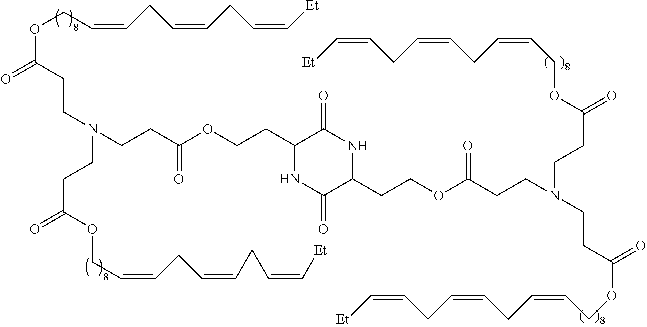 |
| 425 | 2 | C₁₈H₃₁ | 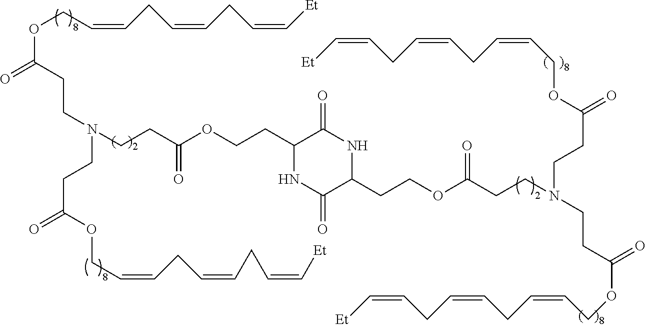 |
| 426 | 3 | C₁₈H₃₁ | 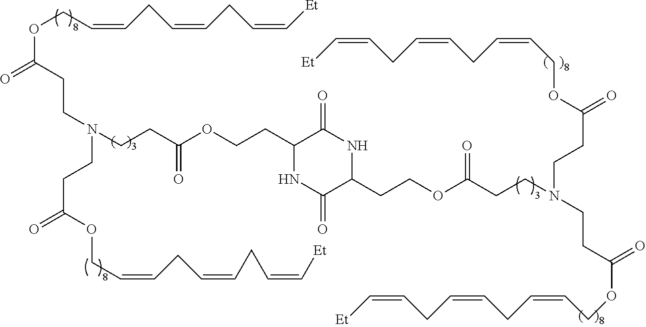 |
| 427 | 1 | substructure a | 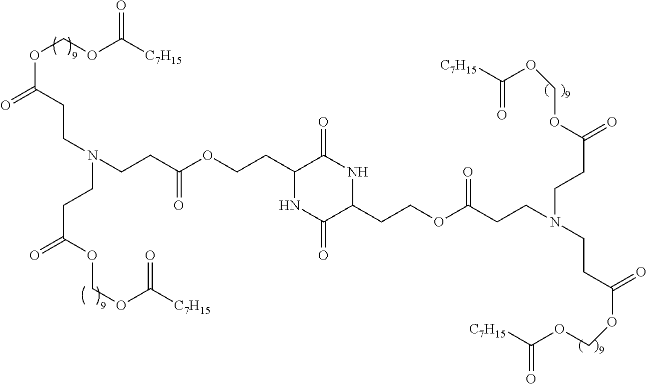 |
| 428 | 2 | substructure a | 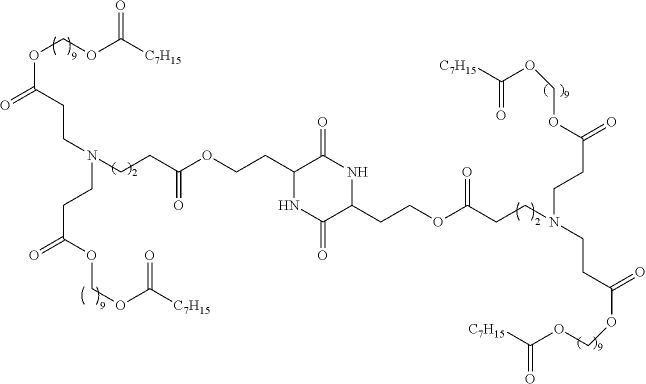 |
| 429 | 3 | substructure a | 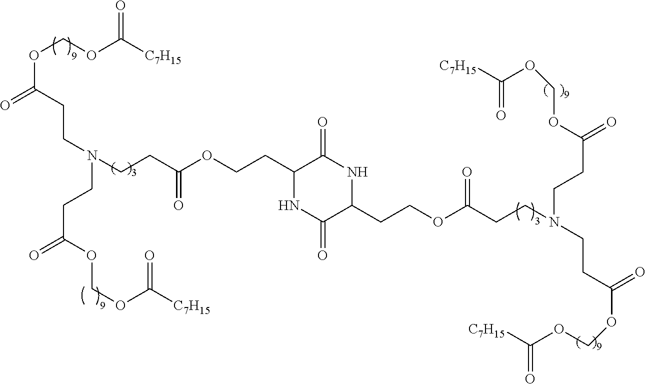 |
| 430 | 1 | substructure b | 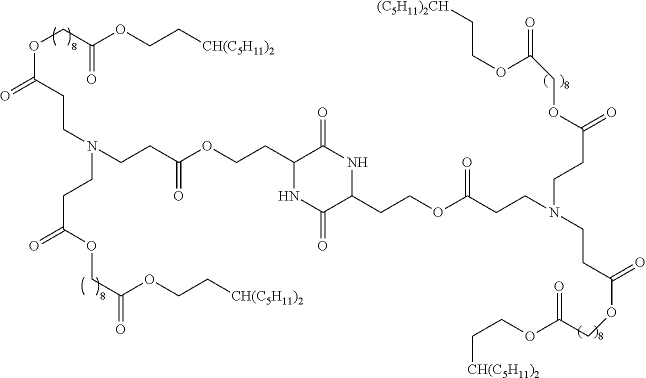 |
| 431 | 2 | substructure b | 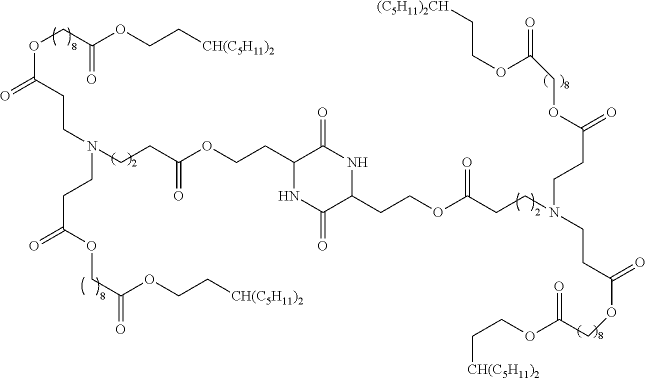 |
| 432 | 3 | substructure b | 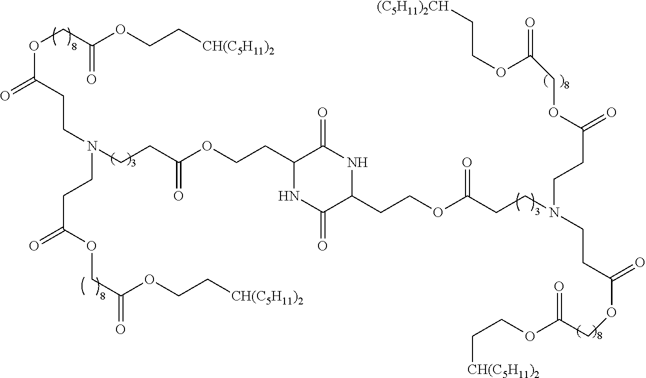 |
| 433 | 1 | —(C₇H₁₄)— CH(CH₃)₂ | 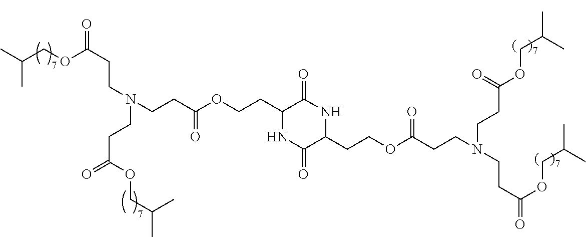 |
| 434 | 2 | —(C₇H₁₄)— CH(CH₃)₂ | 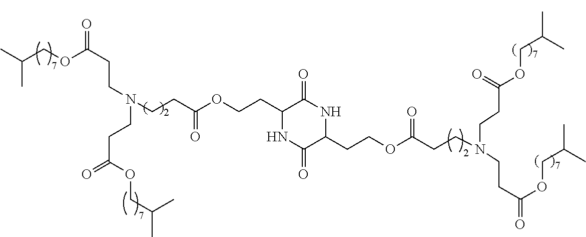 |
| 435 | 3 | —(C₇H₁₄)— CH(CH₃)₂ | 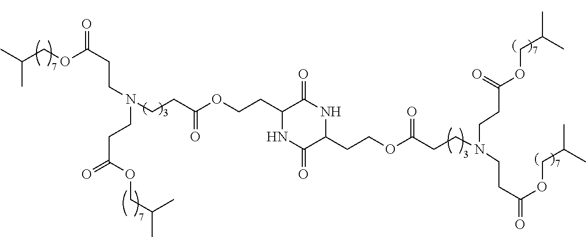 |

In embodiments, a cationic lipid is Compound 397. In embodiments, a cationic lipid is Compound 398. In embodiments, a cationic lipid is Compound 399. In embodiments, a cationic lipid is Compound 400. In embodiments, a cationic lipid is Compound 401. In embodiments, a cationic lipid is Compound 402. In embodiments, a cationic lipid is Compound 403. In embodiments, a cationic lipid is Compound 404. In embodiments, a cationic lipid is Compound 405. In embodiments, a cationic lipid is Compound 406. In embodiments, a cationic lipid is Compound 407. In embodiments, a cationic lipid is Compound 408. In embodiments, a cationic lipid is Compound 409. In embodiments, a cationic lipid is Compound 410. In embodiments, a cationic lipid is Compound 411. In embodiments, a cationic lipid is Compound 412. In embodiments, a cationic lipid is Compound 413. In embodiments, a cationic lipid is Compound 414. In embodiments, a cationic lipid is Compound 415. In embodiments, a cationic lipid is Compound 416. In embodiments, a cationic lipid is Compound 417. In embodiments, a cationic lipid is Compound 418. In embodiments, a cationic lipid is Compound 419. In embodiments, a cationic lipid is Compound 420. In embodiments, a cationic lipid is Compound 421. In embodiments, a cationic lipid is Compound 422. In embodiments, a cationic lipid is Compound 423. In embodiments, a cationic lipid is Compound 424. In embodiments, a cationic lipid is Compound 425. In embodiments, a cationic lipid is Compound 426. In embodiments, a cationic lipid is Compound 427. In embodiments, a cationic lipid is Compound 428. In embodiments, a cationic lipid is Compound 429. In embodiments, a cationic lipid is Compound 430. In embodiments, a cationic lipid is Compound 431. In embodiments, a cationic lipid is Compound 432. In embodiments, a cationic lipid is Compound 433. In embodiments, a cationic lipid is Compound 434. In embodiments, a cationic lipid is Compound 435.

TABLE N
cCC Disulfides-Biodegradable

| | |
|---|---|
| | (III-a) |
| 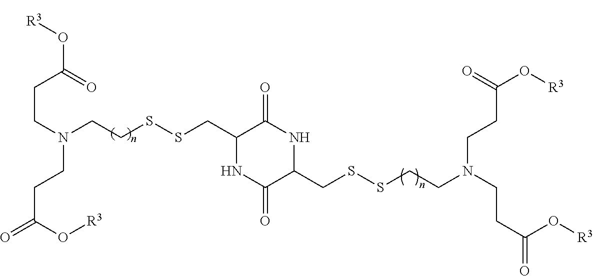 | |

| No. | n | R³ | Structure |
|---|---|---|---|
| 436 | 1 | C₆H₁₃ | 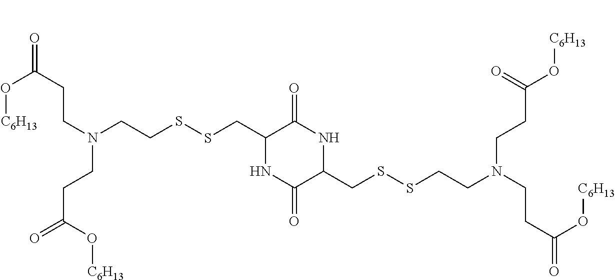 |
| 437 | 2 | C₆H₁₃ | 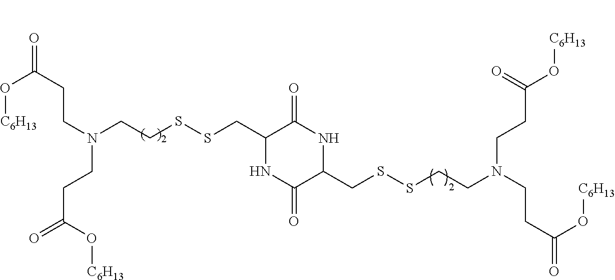 |
| 438 | 3 | C₆H₁₃ | 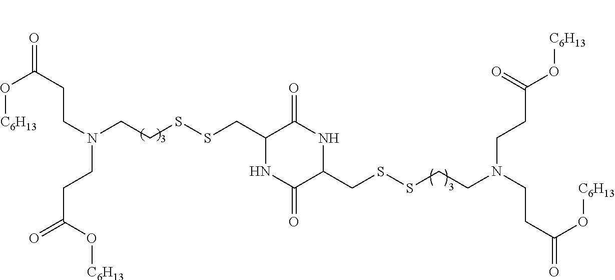 |
| 439 | 1 | C₈H₁₇ | 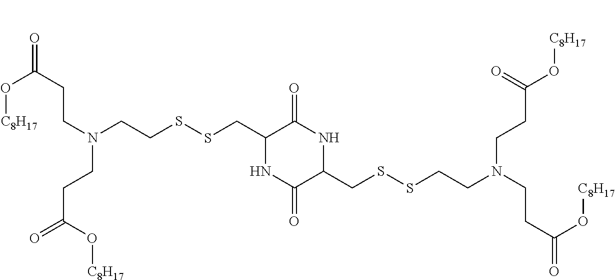 |
| 440 | 2 | C₈H₁₇ | 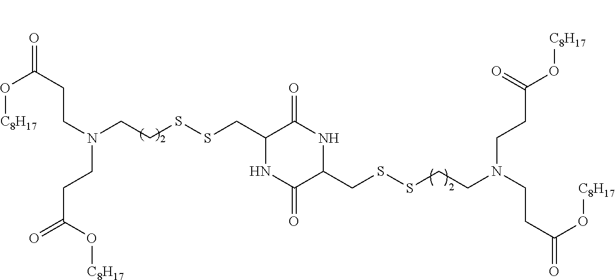 |
| 441 | 3 | C₈H₁₇ | 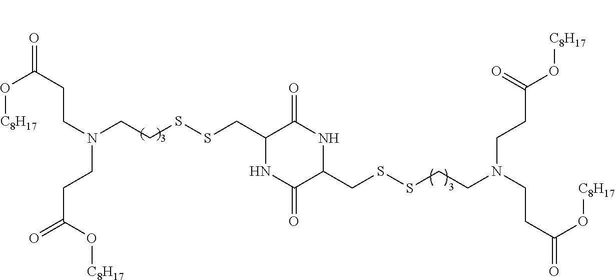 |
| 442 | 1 | C₁₀H₂₁ | 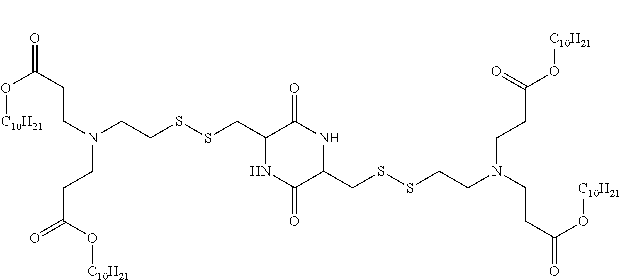 |
| 443 | 2 | C₁₀H₂₁ | 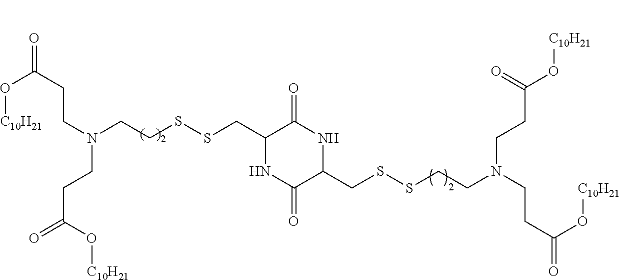 |
| 444 | 3 | C₁₀H₂₁ | 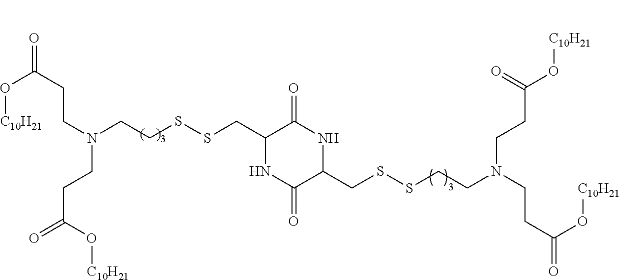 |
| 445 | 1 | C₁₂H₂₅ | 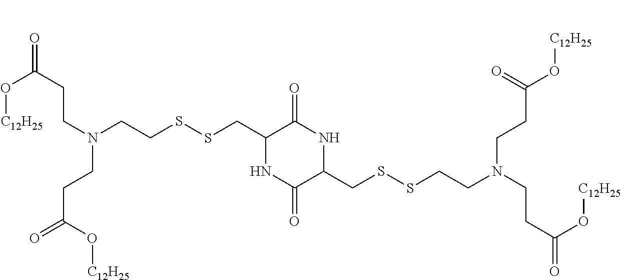 |
| 446 | 2 | C₁₂H₂₅ | 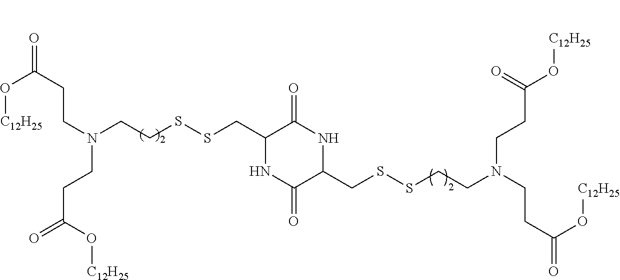 |
| 447 | 3 | C₁₂H₂₅ | 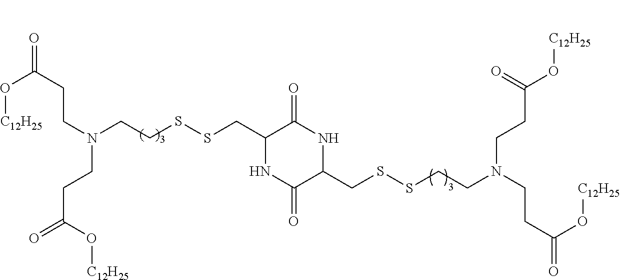 |
| 448 | 1 | C₁₄H₂₉ | 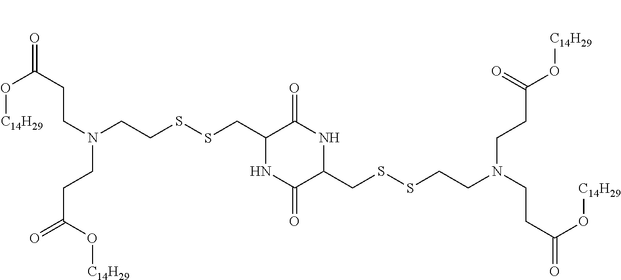 |
| 449 | 2 | C₁₄H₂₉ | 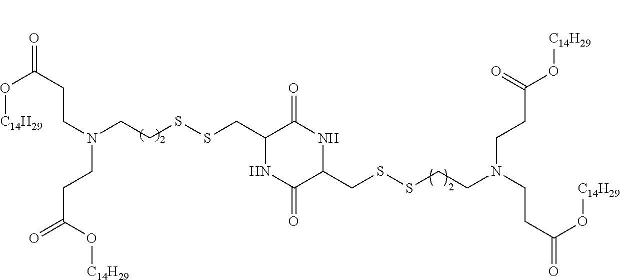 |
| 450 | 3 | C₁₄H₂₉ | 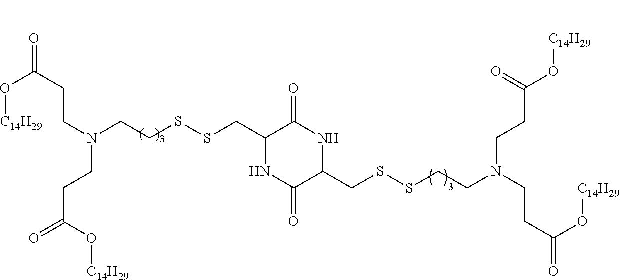 |
| 451 | 1 | C₁₆H₃₃ | 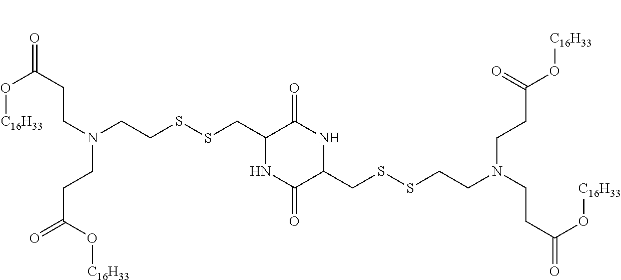 |
| 452 | 2 | C₁₆H₃₃ | 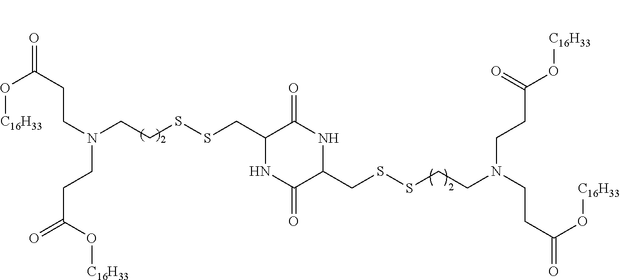 |
| 453 | 3 | C₁₆H₃₃ | 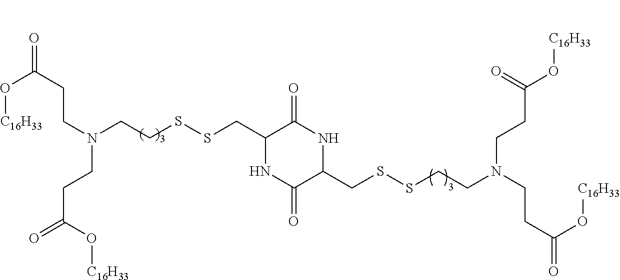 |
| 454 | 1 | C₁₈H₃₇ | 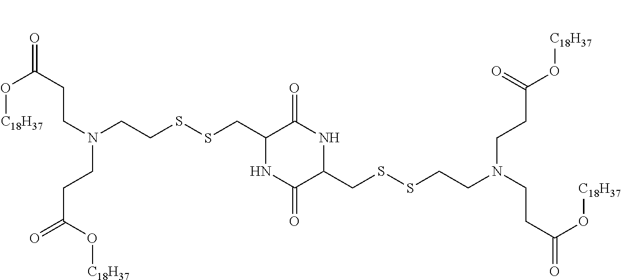 |
| 455 | 2 | C₁₈H₃₇ | 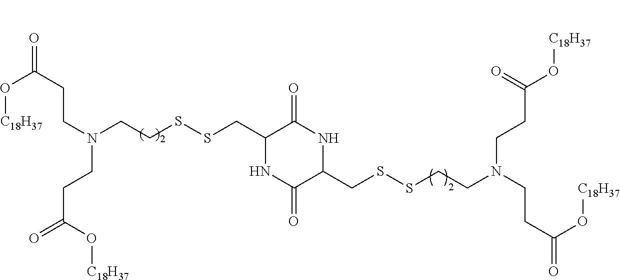 |
| 456 | 3 | C₁₈H₃₇ | 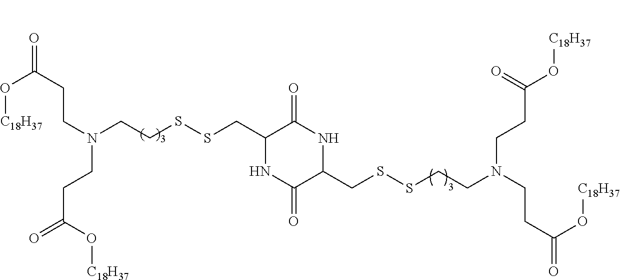 |
| 457 | 1 | C₁₈H₃₃ | 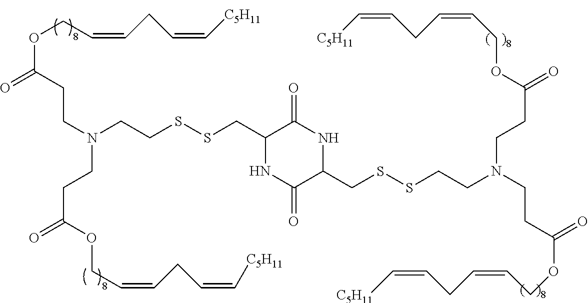 |
| 458 | 2 | C₁₈H₃₃ | 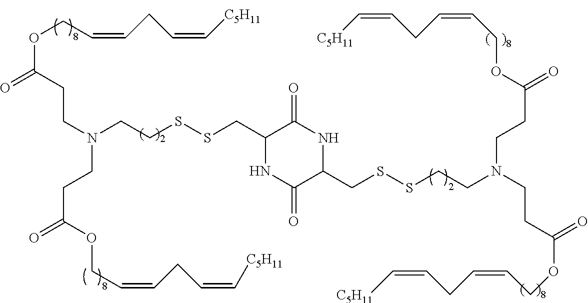 |
| 459 | 3 | C₁₈H₃₃ | 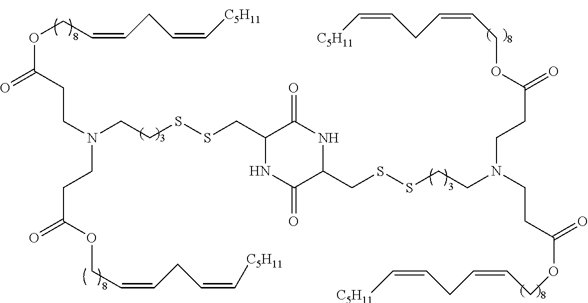 |
| 460 | 1 | C₁₈H₃₅ | 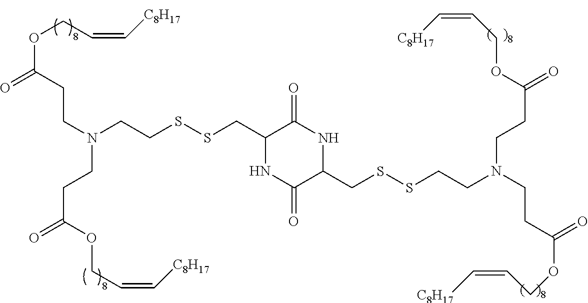 |
| 461 | 2 | C₁₈H₃₅ | 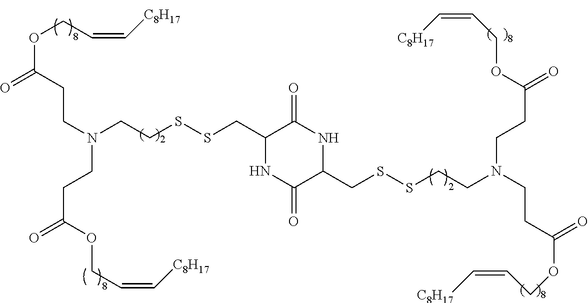 |
| 462 | 3 | C₁₈H₃₅ | 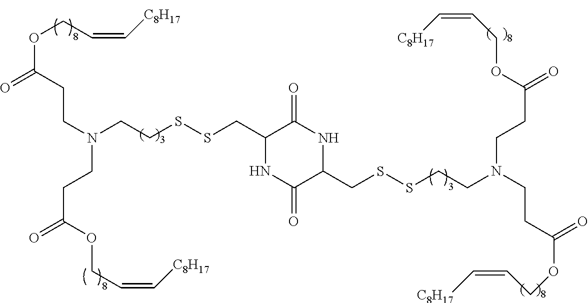 |
| 463 | 1 | C₁₈H₃₁ | 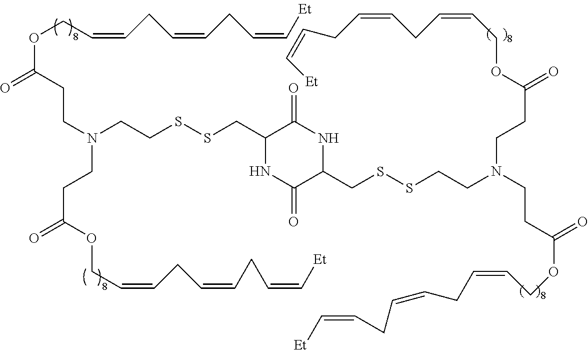 |
| 464 | 2 | C₁₈H₃₁ | 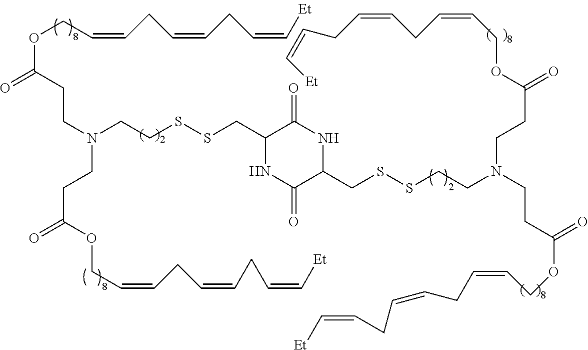 |
| 465 | 3 | C₁₈H₃₁ | 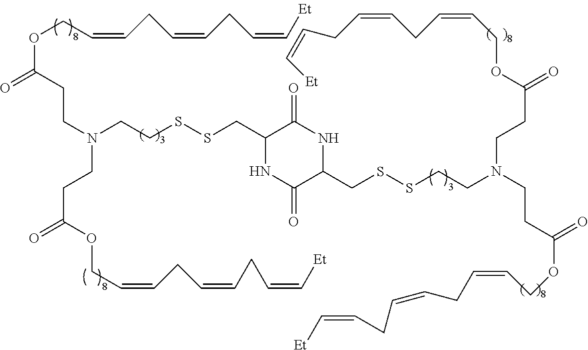 |
| 466 | 1 | substructure a | 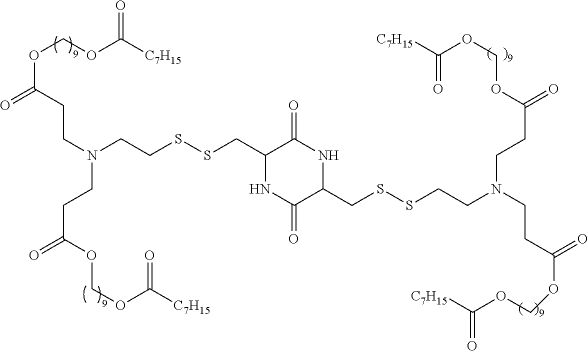 |
| 467 | 2 | substructure a | 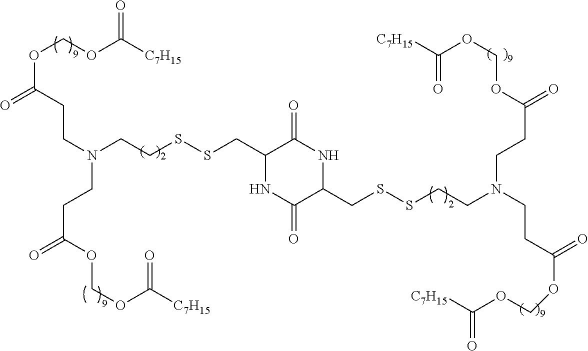 |
| 468 | 3 | substructure a | 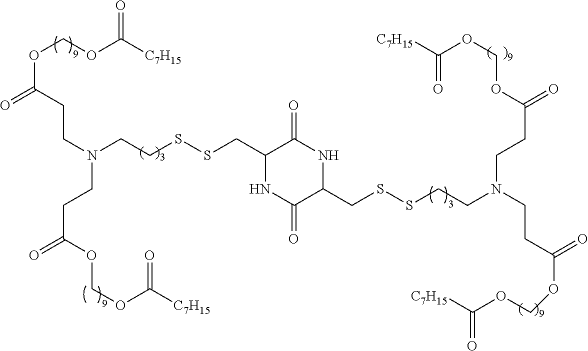 |
| 469 | 1 | substructure b | 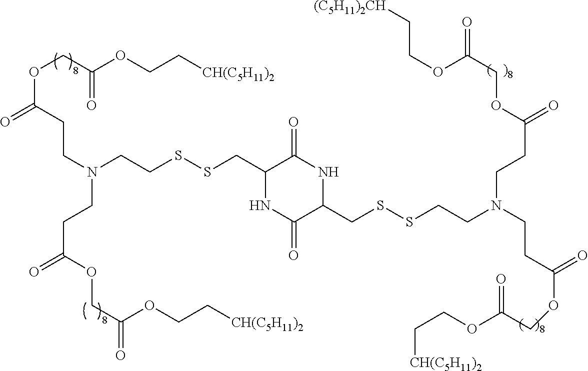 |
| 470 | 2 | substructure b | 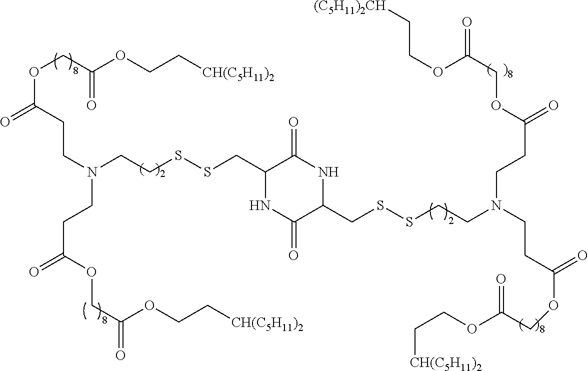 |
| 471 | 3 | substructure b | 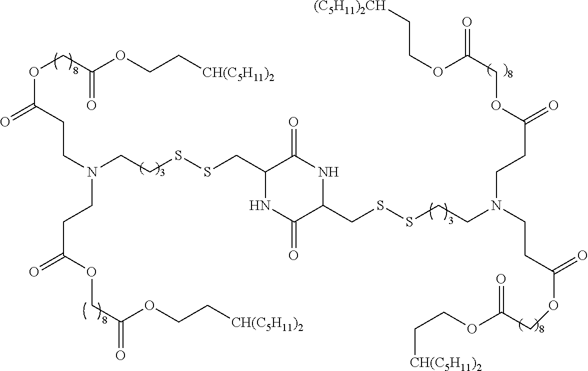 |
| 472 | 1 | —(C₇H₁₄)— CH(CH₃)₂ | 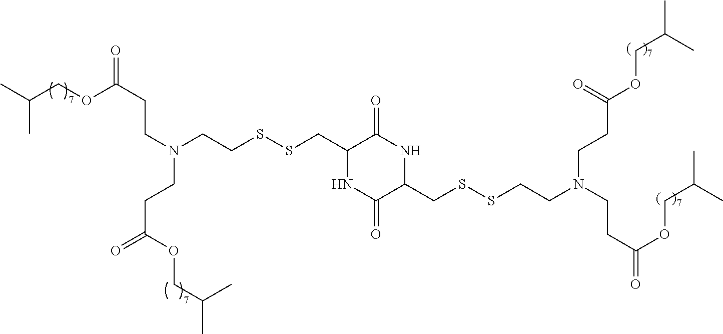 |
| 473 | 2 | —(C₇H₁₄)— CH(CH₃)₂ | 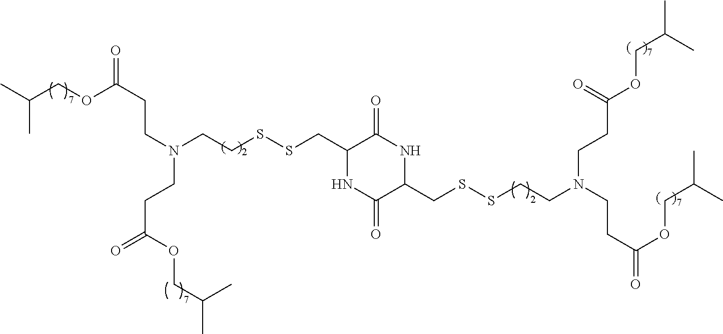 |
| 474 | 3 | —(C₇H₁₄)— CH(CH₃)₂ | 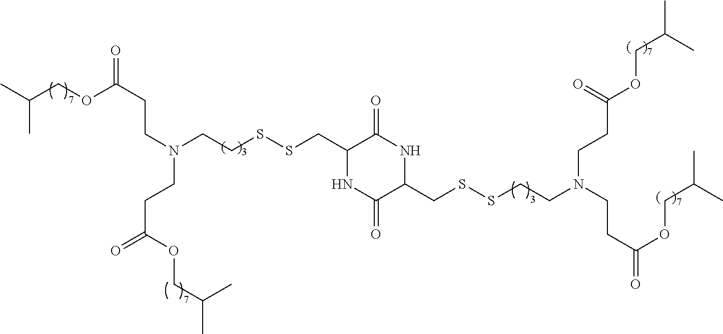 |

In embodiments, a cationic lipid is Compound 436. In embodiments, a cationic lipid is Compound 437. In embodiments, a cationic lipid is Compound 438. In embodiments, a cationic lipid is Compound 439. In embodiments, a cationic lipid is Compound 440. In embodiments, a cationic lipid is Compound 441. In embodiments, a cationic lipid is Compound 442. In embodiments, a cationic lipid is Compound 443. In embodiments, a cationic lipid is Compound 444. In embodiments, a cationic lipid is Compound 445. In embodiments, a cationic lipid is Compound 446. In embodiments, a cationic lipid is Compound 447. In embodiments, a cationic lipid is Compound 448. In embodiments, a cationic lipid is Compound 449. In embodiments, a cationic lipid is Compound 450. In embodiments, a cationic lipid is Compound 451. In embodiments, a cationic lipid is Compound 452. In embodiments, a cationic lipid is Compound 453. In embodiments, a cationic lipid is Compound 454. In embodiments, a cationic lipid is Compound 455. In embodiments, a cationic lipid is Compound 456. In embodiments, a cationic lipid is Compound 457. In embodiments, a cationic lipid is Compound 458. In embodiments, a cationic lipid is Compound 459. In embodiments, a cationic lipid is Compound 460. In embodiments, a cationic lipid is Compound 461. In embodiments, a cationic lipid is Compound 462. In embodiments, a cationic lipid is Compound 463. In embodiments, a cationic lipid is Compound 464. In embodiments, a cationic lipid is Compound 465. In embodiments, a cationic lipid is Compound 466. In embodiments, a cationic lipid is Compound 467. In embodiments, a cationic lipid is Compound 468. In embodiments, a cationic lipid is Compound 469. In embodiments, a cationic lipid is Compound 470. In embodiments, a cationic lipid is Compound 471. In embodiments, a cationic lipid is Compound 472. In embodiments, a cationic lipid is Compound 473. In embodiments, a cationic lipid is Compound 474.

TABLE O
cCC Thioesters-Biodegradable

| | |
|---|---|
| | (III-b) |
| 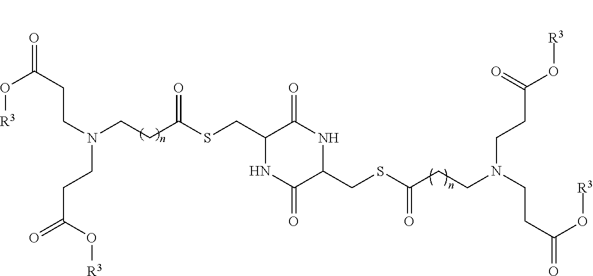 | |

| No. | n | R³ | Structure |
|---|---|---|---|
| 475 | 1 | C₆H₁₃ | 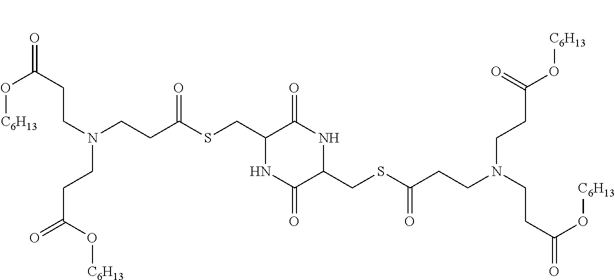 |
| 476 | 2 | C₆H₁₃ | 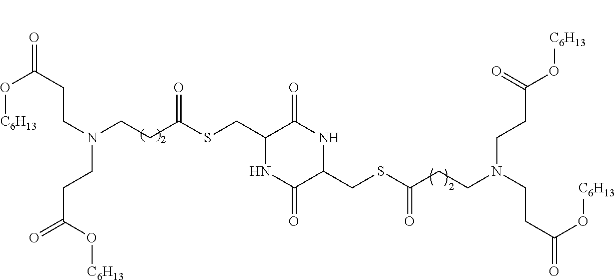 |
| 477 | 3 | C₆H₁₃ | 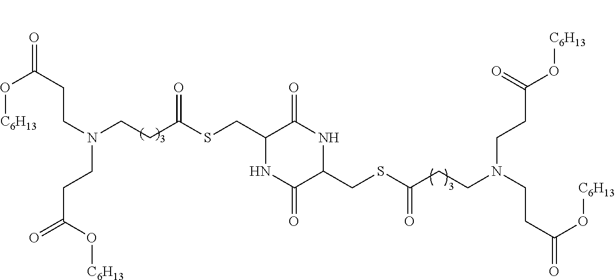 |
| 478 | 1 | C₈H₁₇ | 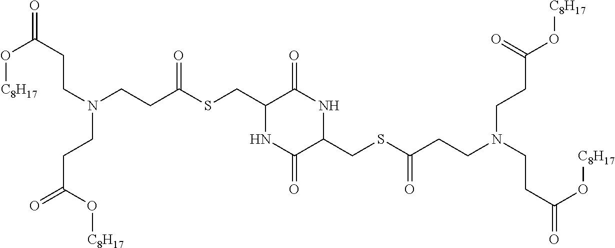 |
| 479 | 2 | C₈H₁₇ | 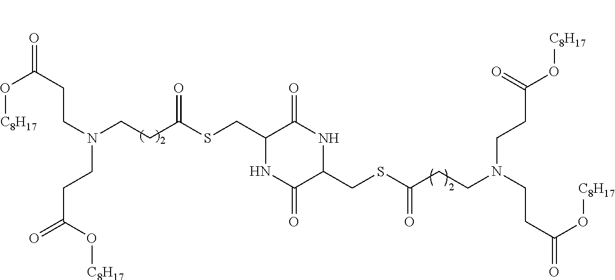 |
| 480 | 3 | C₈H₁₇ | 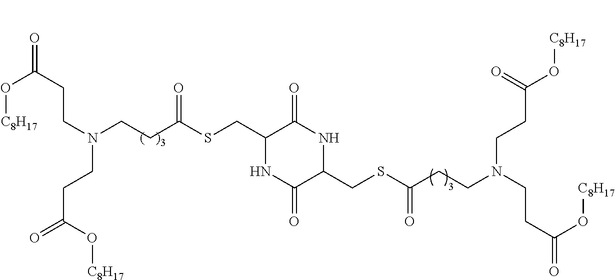 |
| 481 | 1 | C₁₀H₂₁ | 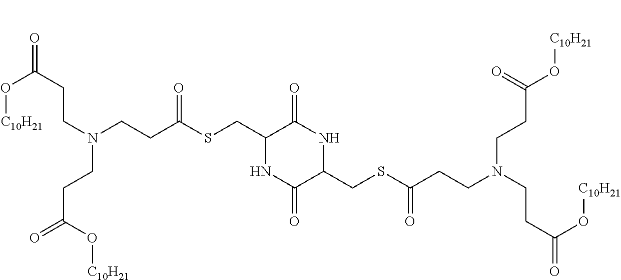 |
| 482 | 2 | C₁₀H₂₁ | 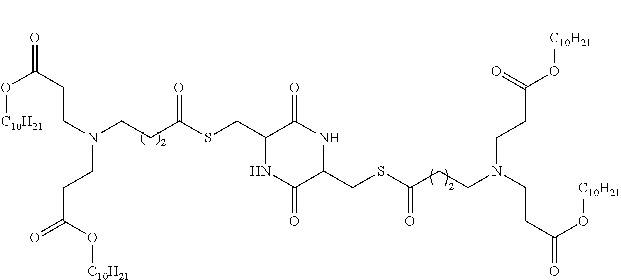 |
| 483 | 3 | C₁₀H₂₁ | 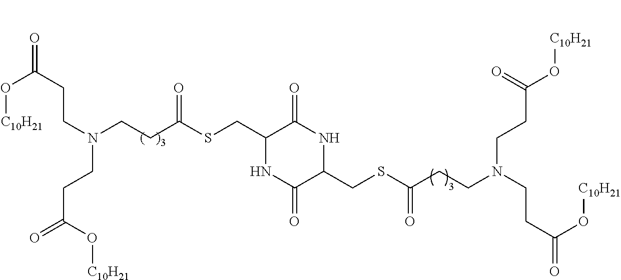 |
| 484 | 1 | C₁₂H₂₅ | 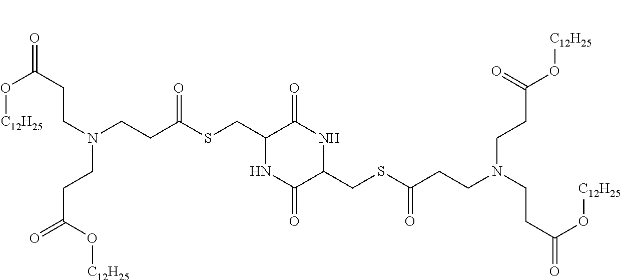 |
| 485 | 2 | C₁₂H₂₅ | 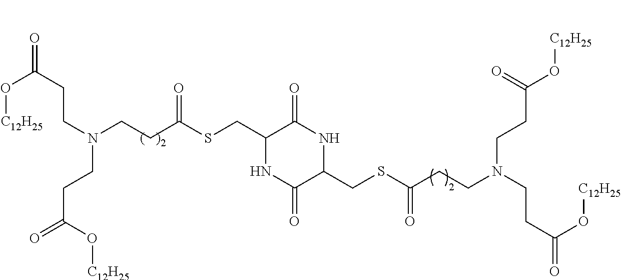 |
| 486 | 3 | C₁₂H₂₅ | 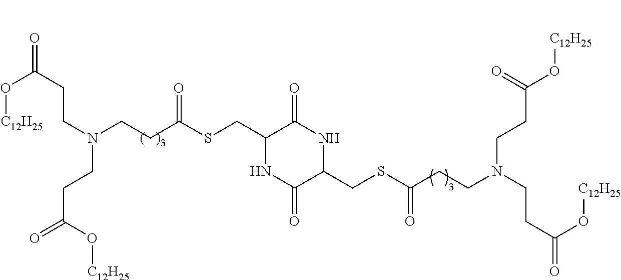 |
| 487 | 1 | C₁₄H₂₉ | 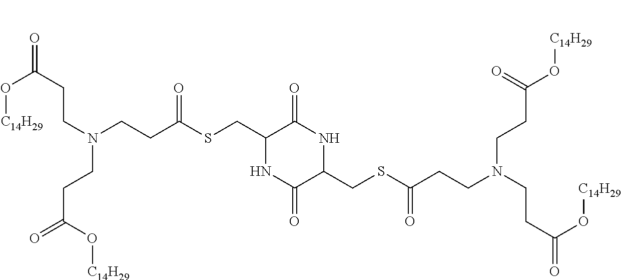 |
| 488 | 2 | C₁₄H₂₉ | 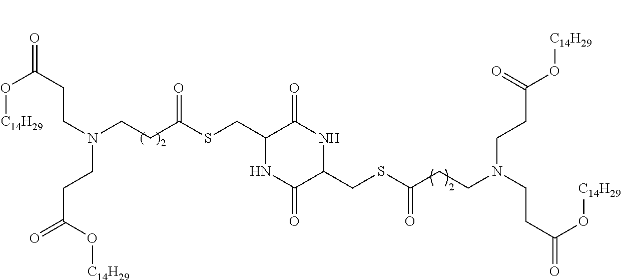 |
| 489 | 3 | C₁₄H₂₉ | 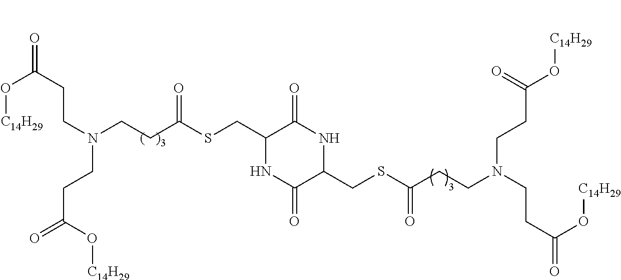 |
| 490 | 1 | C₁₆H₃₃ | 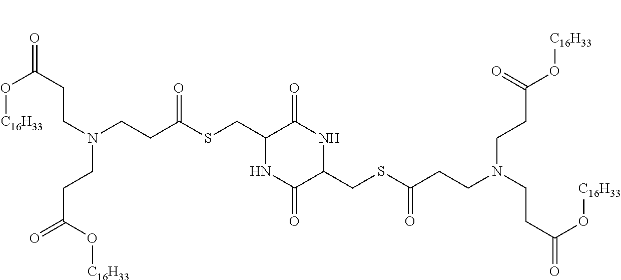 |
| 491 | 2 | C₁₆H₃₃ | 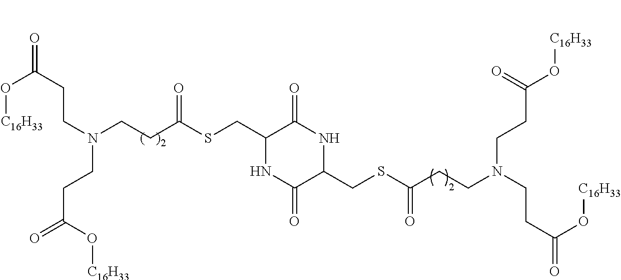 |
| 492 | 3 | C₁₆H₃₃ | 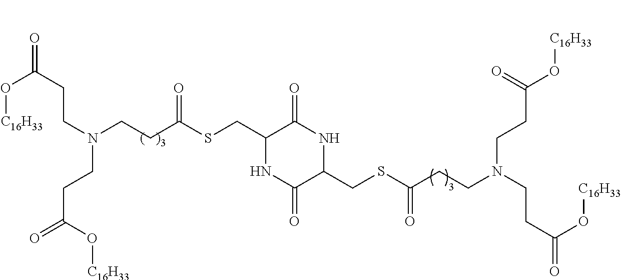 |
| 493 | 1 | C₁₈H₃₇ | 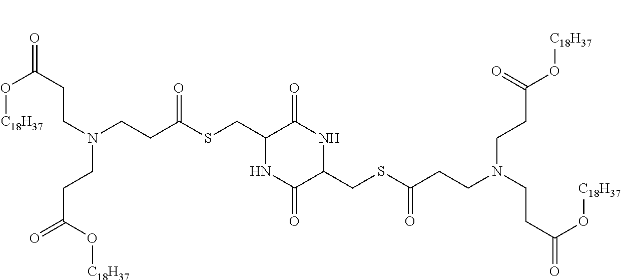 |
| 494 | 2 | C₁₈H₃₇ | 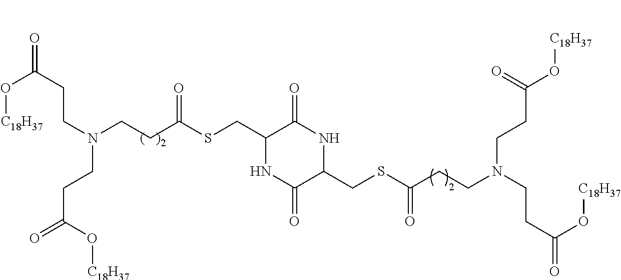 |
| 495 | 3 | C₁₈H₃₇ | 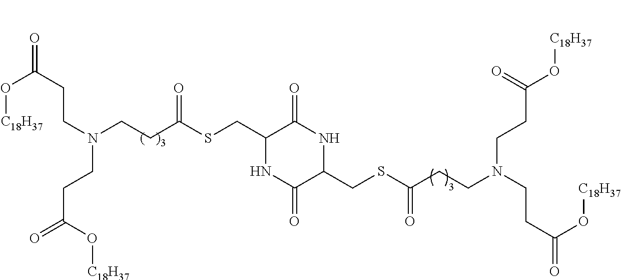 |
| 496 | 1 | C₁₈H₃₃ | 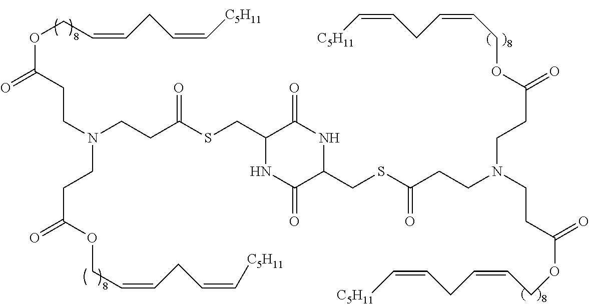 |
| 497 | 2 | C₁₈H₃₃ | 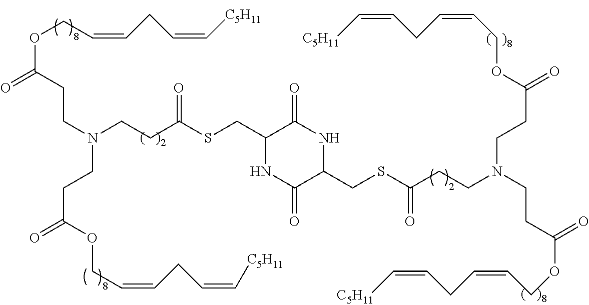 |
| 498 | 3 | C₁₈H₃₃ | 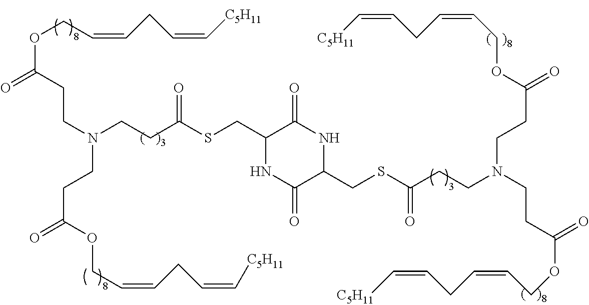 |
| 499 | 1 | C₁₈H₃₅ | 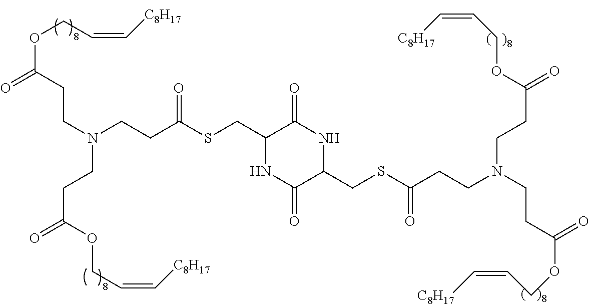 |
| 500 | 2 | C₁₈H₃₅ | 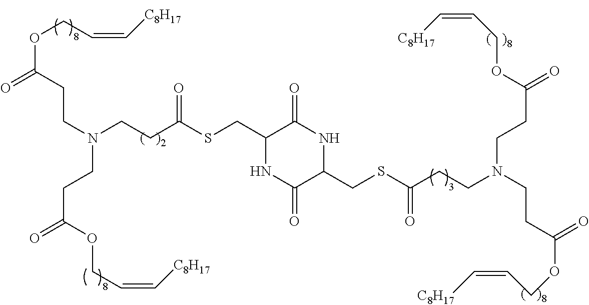 |
| 501 | 3 | C₁₈H₃₅ | 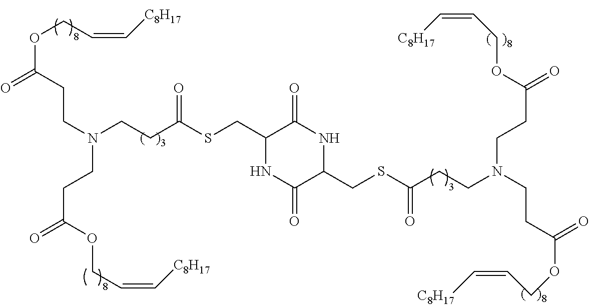 |
| 502 | 1 | C₁₈H₃₁ | 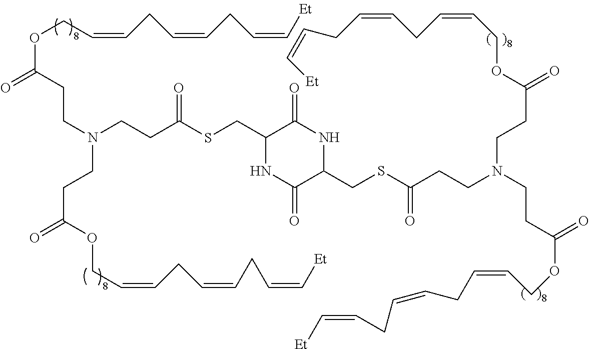 |
| 503 | 2 | C₁₈H₃₁ | 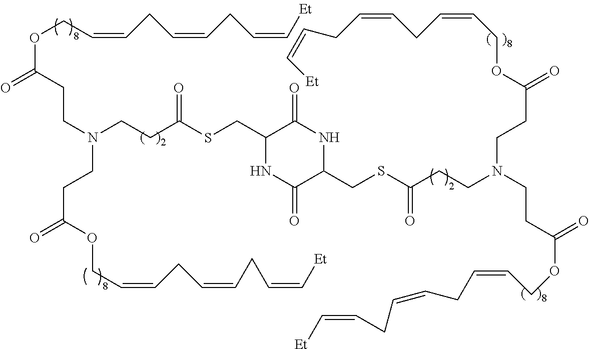 |
| 504 | 3 | C₁₈H₃₁ | 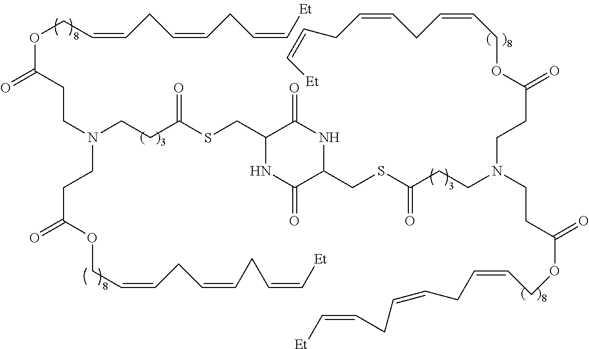 |
| 505 | 1 | substructure a | 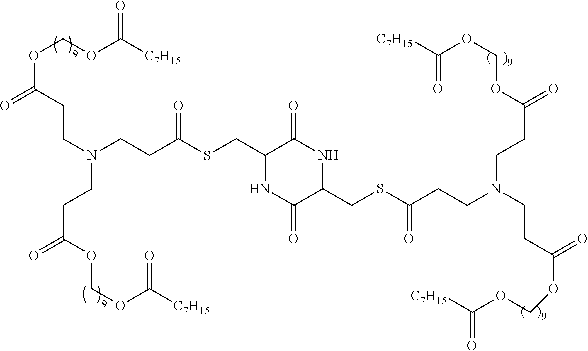 |
| 506 | 2 | substructure a | 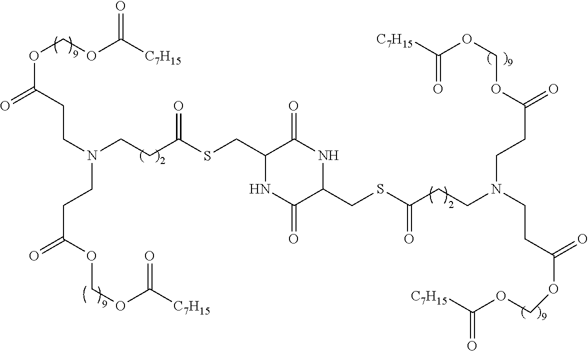 |
| 507 | 3 | substructure a | 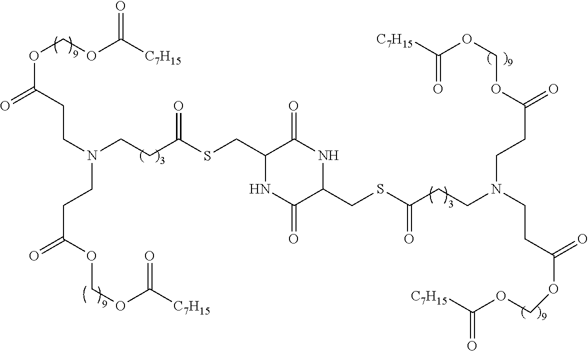 |
| 508 | 1 | substructure b | 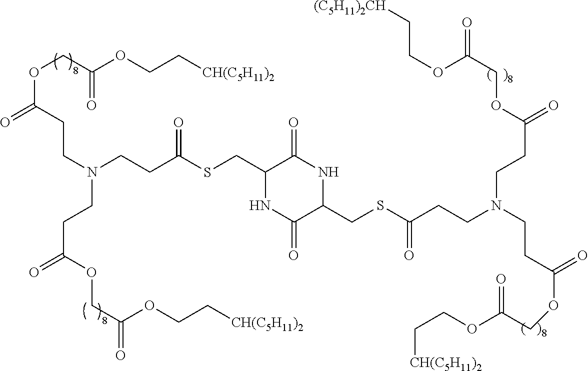 |
| 509 | 2 | substructure b | 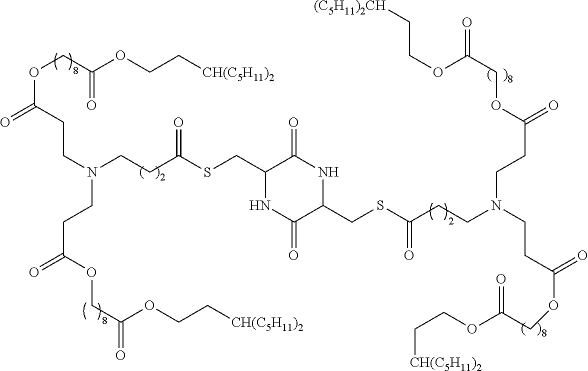 |
| 510 | 3 | substructure b | 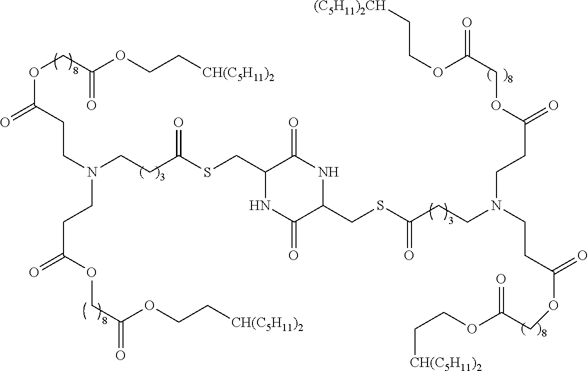 |
| 511 | 1 | —(C₇H₁₄)— CH(CH₃)₂ | 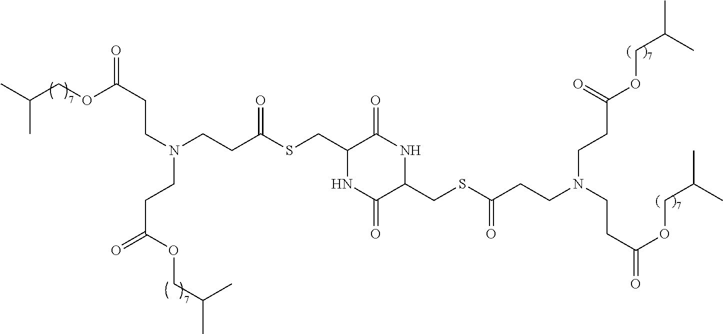 |
| 512 | 2 | —(C₇H₁₄)— CH(CH₃)₂ | 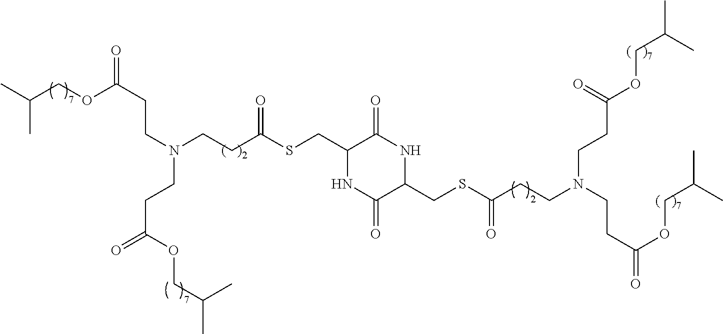 |
| 513 | 3 | —(C₇H₁₄)— CH(CH₃)₂ | 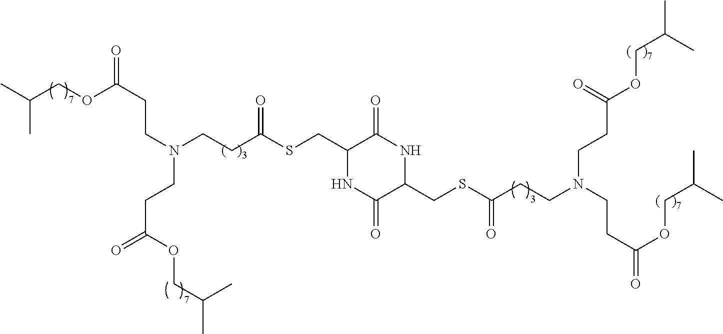 |

In embodiments, a cationic lipid is Compound 475. In embodiments, a cationic lipid is Compound 476. In embodiments, a cationic lipid is Compound 477. In embodiments, a cationic lipid is Compound 478. In embodiments, a cationic lipid is Compound 479. In embodiments, a cationic lipid is Compound 480. In embodiments, a cationic lipid is Compound 481. In embodiments, a cationic lipid is Compound 482. In embodiments, a cationic lipid is Compound 483. In embodiments, a cationic lipid is Compound 484. In embodiments, a cationic lipid is Compound 485. In embodiments, a cationic lipid is Compound 486. In embodiments, a cationic lipid is Compound 487. In embodiments, a cationic lipid is Compound 488. In embodiments, a cationic lipid is Compound 489. In embodiments, a cationic lipid is Compound 490. In embodiments, a cationic lipid is Compound 491. In embodiments, a cationic lipid is Compound 492. In embodiments, a cationic lipid is Compound 493. In embodiments, a cationic lipid is Compound 494. In embodiments, a cationic lipid is Compound 495. In embodiments, a cationic lipid is Compound 496. In embodiments, a cationic lipid is Compound 497. In embodiments, a cationic lipid is Compound 498. In embodiments, a cationic lipid is Compound 499. In embodiments, a cationic lipid is Compound 500. In embodiments, a cationic lipid is Compound 501. In embodiments, a cationic lipid is Compound 502. In embodiments, a cationic lipid is Compound 503. In embodiments, a cationic lipid is Compound 504. In embodiments, a cationic lipid is Compound 505. In embodiments, a cationic lipid is Compound 506. In embodiments, a cationic lipid is Compound 507. In embodiments, a cationic lipid is Compound 508. In embodiments, a cationic lipid is Compound 509. In embodiments, a cationic lipid is Compound 510. In embodiments, a cationic lipid is Compound 511. In embodiments, a cationic lipid is Compound 512. In embodiments, a cationic lipid is Compound 513.

TABLE P
cSS Esters-Biodegradable

| | |
|---|---|
| | (III-c-1) |
| 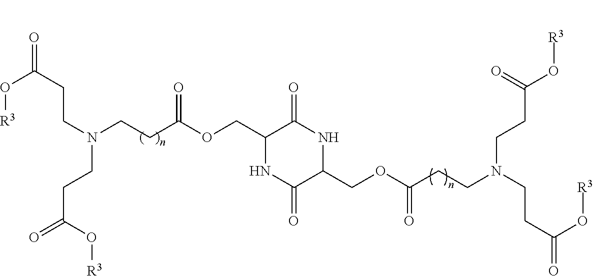 | |

| No. | n | R³ | Structure |
|---|---|---|---|
| 514 | 1 | C₆H₁₃ | 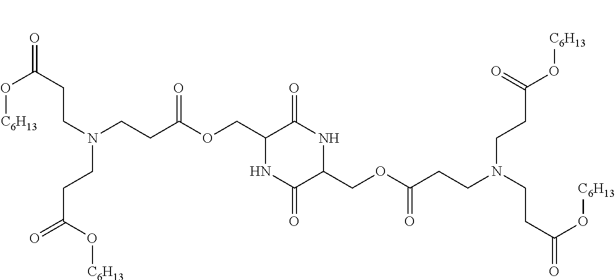 |
| 515 | 2 | C₆H₁₃ | 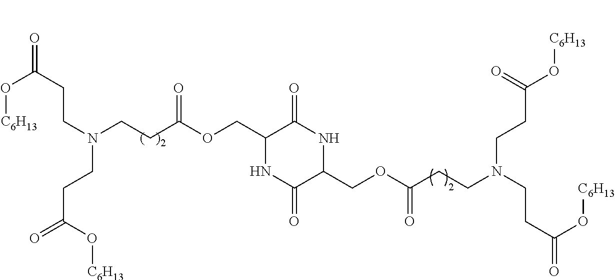 |
| 516 | 3 | C₆H₁₃ | 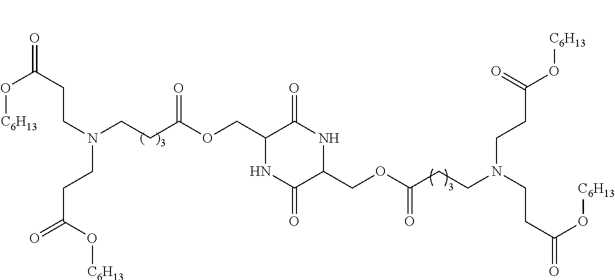 |
| 517 | 1 | C₈H₁₇ | 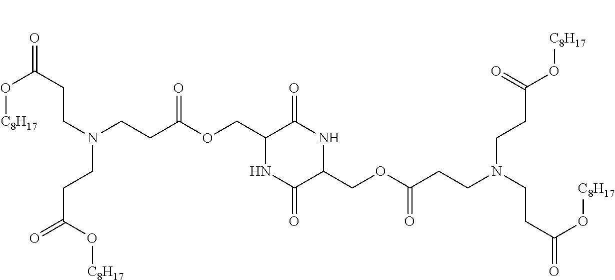 |
| 518 | 2 | C₈H₁₇ | 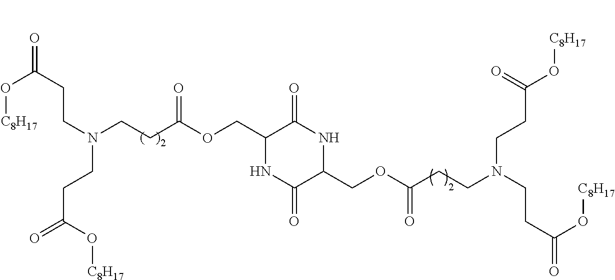 |
| 519 | 3 | C₈H₁₇ | 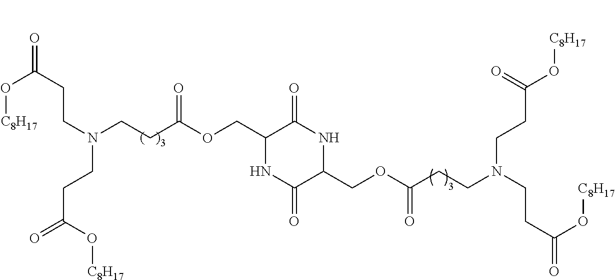 |
| 520 | 1 | C₁₀H₂₁ | 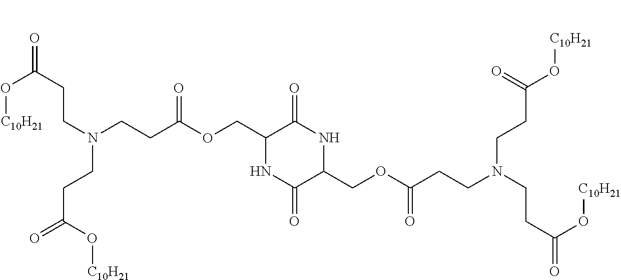 |
| 521 | 2 | C₁₀H₂₁ | 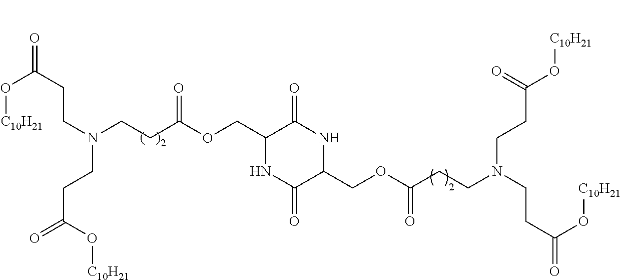 |
| 522 | 3 | C₁₀H₂₁ | 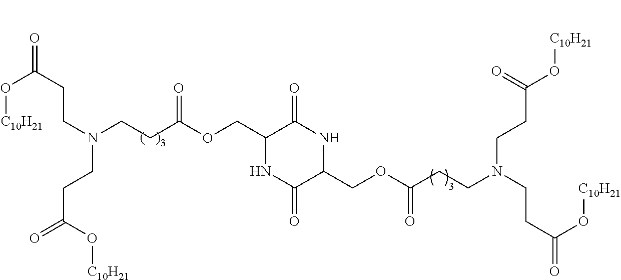 |
| 523 | 1 | C₁₂H₂₅ | 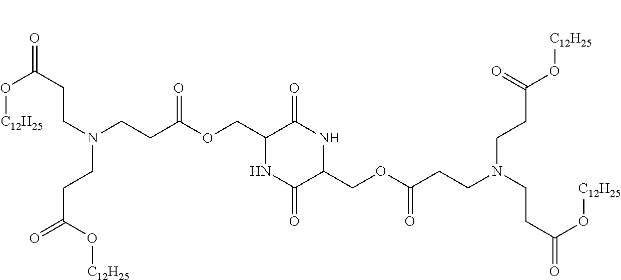 |
| 524 | 2 | C₁₂H₂₅ | 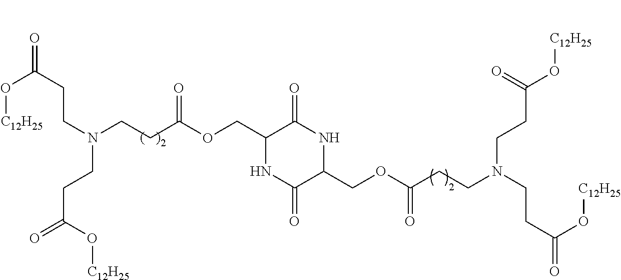 |
| 525 | 3 | C₁₂H₂₅ | 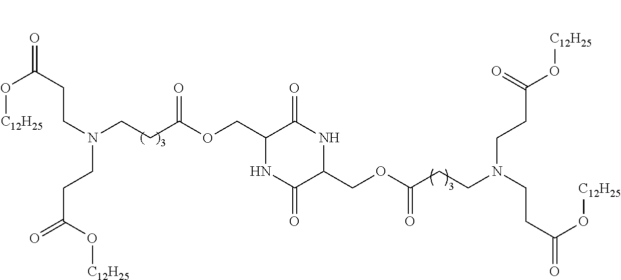 |
| 526 | 1 | C₁₄H₂₉ | 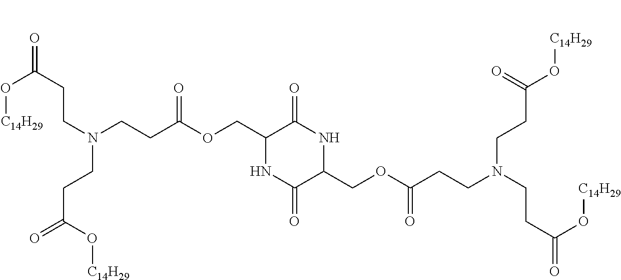 |
| 527 | 2 | C₁₄H₂₉ | 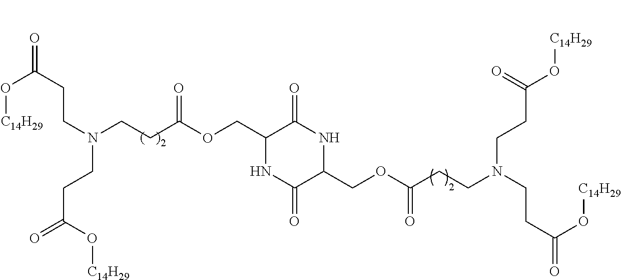 |
| 528 | 3 | C₁₄H₂₉ | 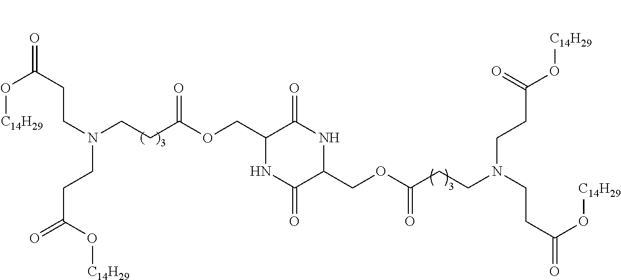 |
| 529 | 1 | C₁₆H₃₃ | 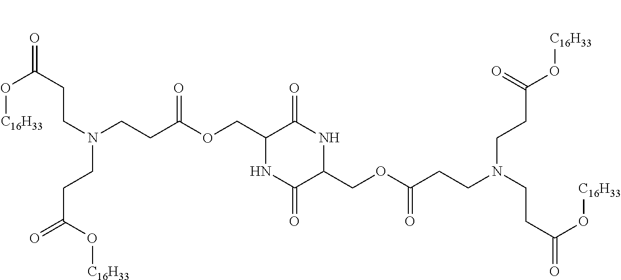 |
| 530 | 2 | C₁₆H₃₃ | 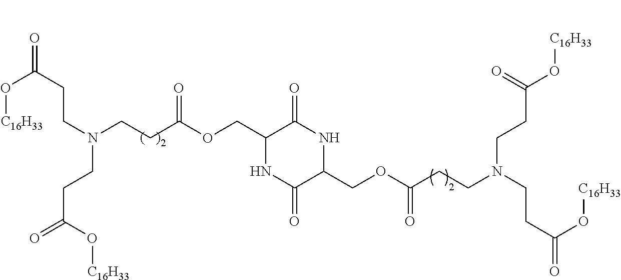 |
| 531 | 3 | C₁₆H₃₃ | 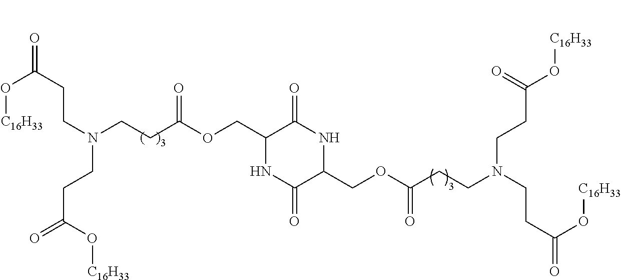 |
| 532 | 1 | C₁₈H₃₇ | 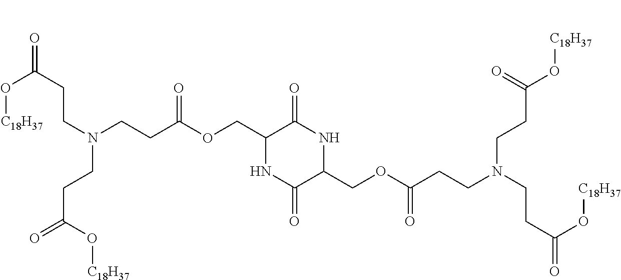 |
| 533 | 2 | C₁₈H₃₇ | 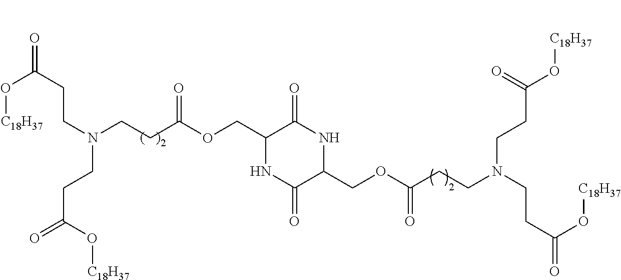 |
| 534 | 3 | C₁₈H₃₇ | 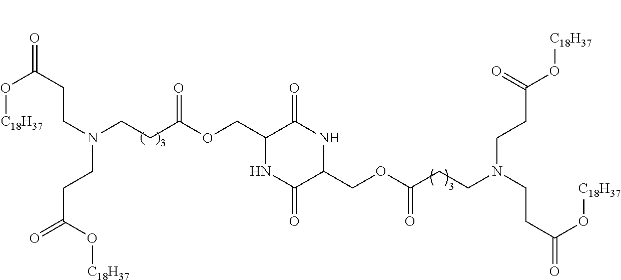 |
| 535 | 1 | C₁₈H₃₃ | 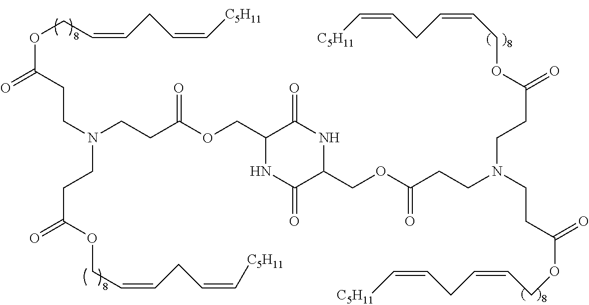 |
| 536 | 2 | C₁₈H₃₃ | 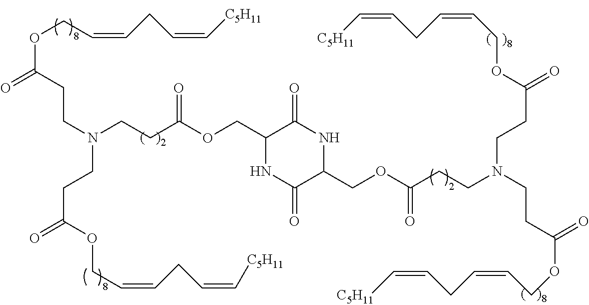 |
| 537 | 3 | C₁₈H₃₃ | 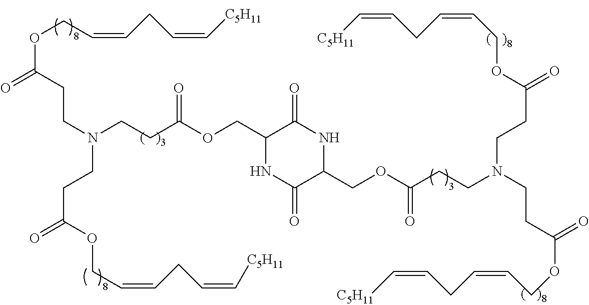 |
| 538 | 1 | C₁₈H₃₅ | 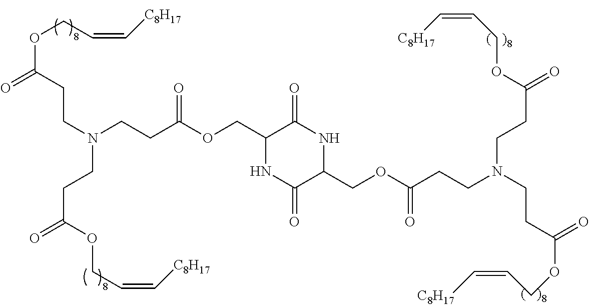 |
| 539 | 2 | C₁₈H₃₅ | 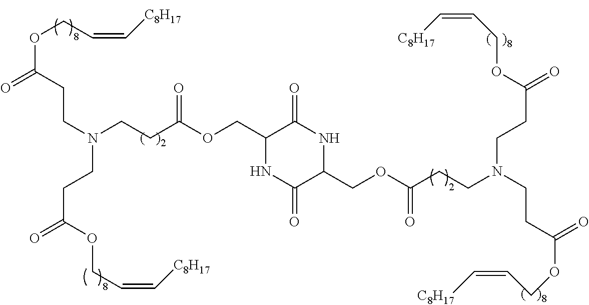 |
| 540 | 3 | C₁₈H₃₅ | 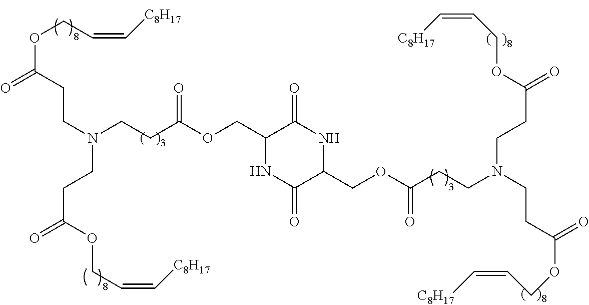 |
| 541 | 1 | C₁₈H₃₁ | 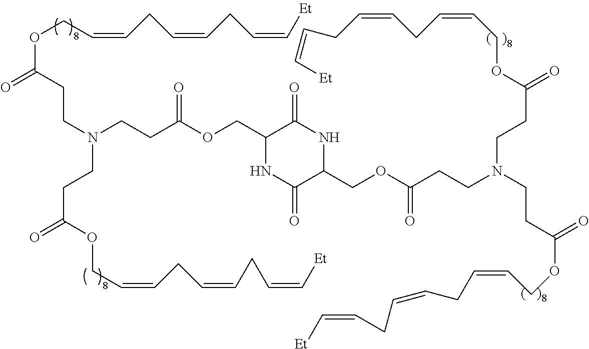 |
| 542 | 2 | C₁₈H₃₁ | 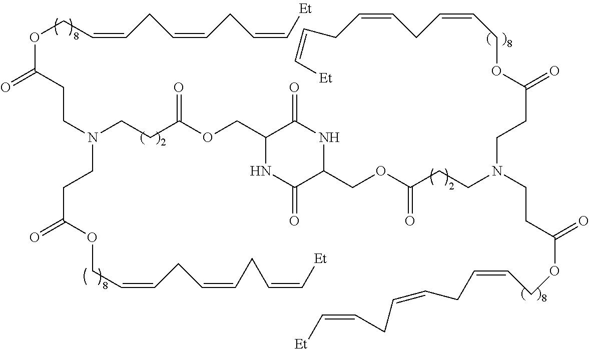 |
| 543 | 3 | C₁₈H₃₁ | 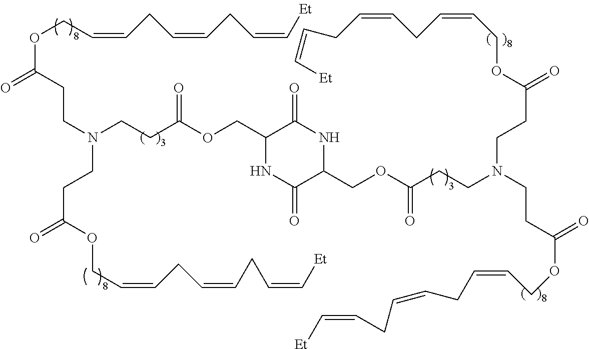 |
| 544 | 1 | substructure a | 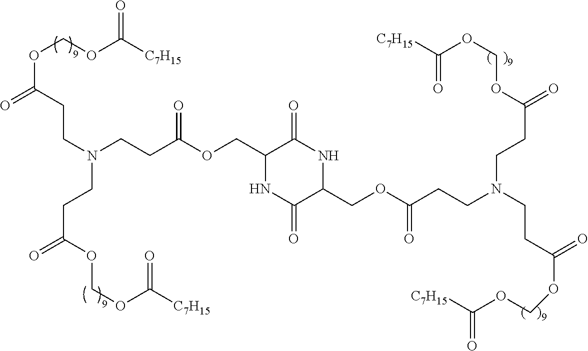 |
| 545 | 2 | substructure a | 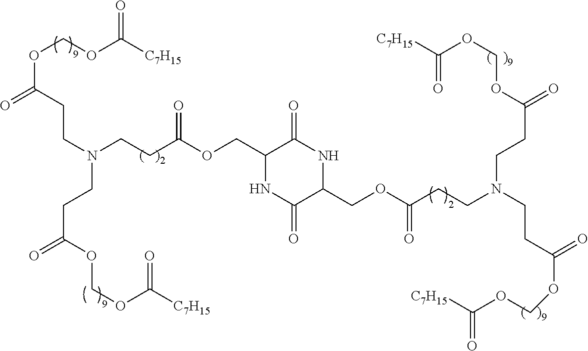 |
| 546 | 3 | substructure a | 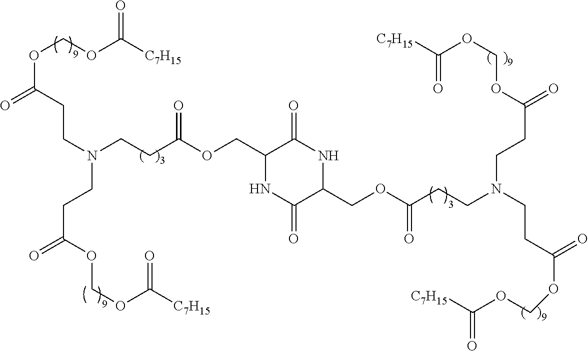 |
| 547 | 1 | substructure b | 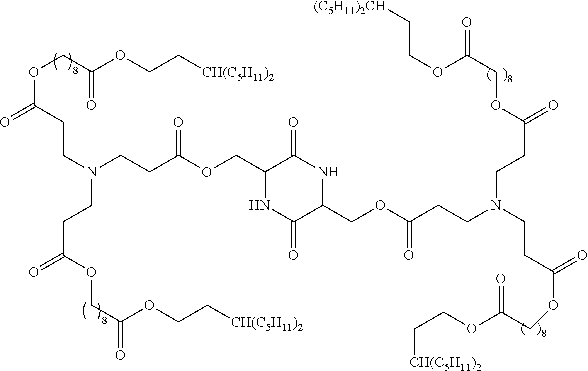 |
| 548 | 2 | substructure b | 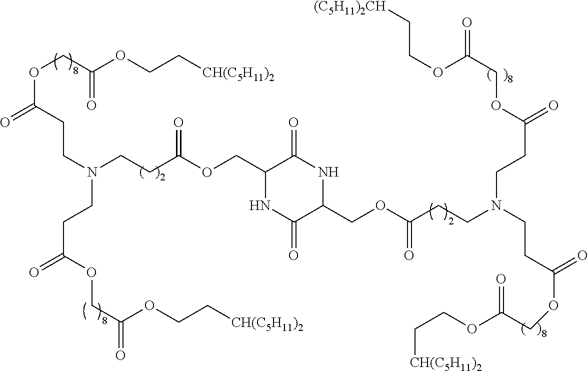 |
| 549 | 3 | substructure b | 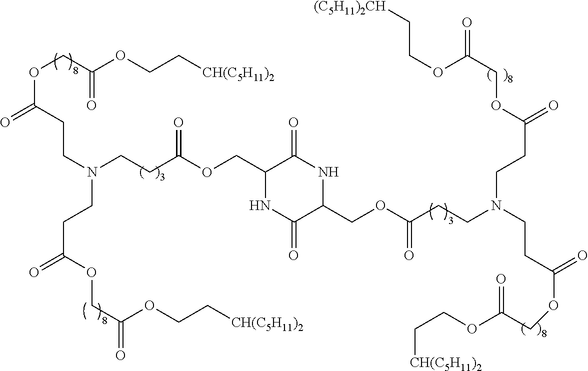 |
| 550 | 1 | —(C₇H₁₄)— CH(CH₃)₂ | 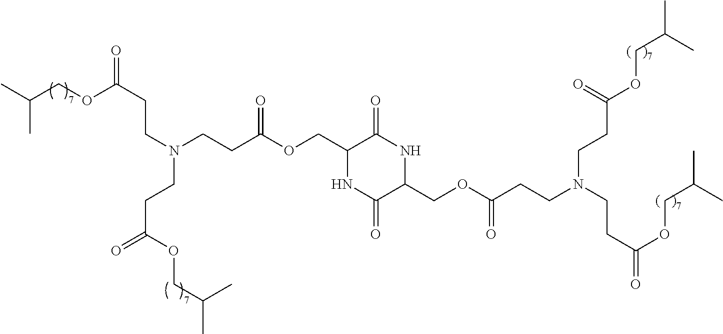 |
| 551 | 2 | —(C₇H₁₄)— CH(CH₃)₂ | 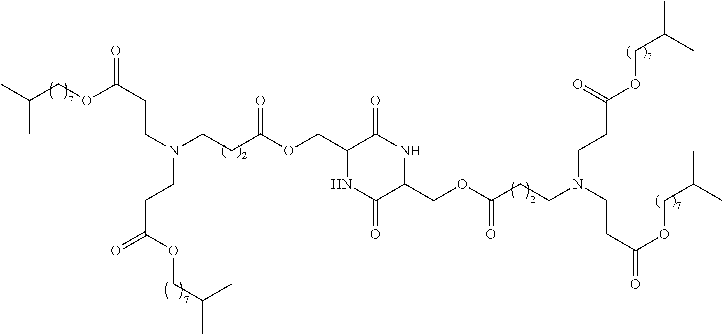 |
| 552 | 3 | —(C₇H₁₄)— CH(CH₃)₂ | 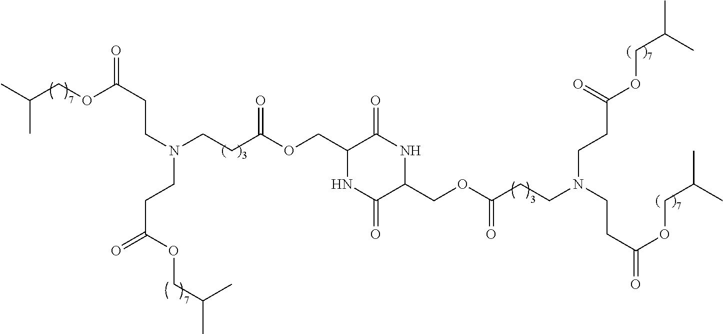 |

In embodiments, a cationic lipid is Compound 514. In embodiments, a cationic lipid is Compound 515. In embodiments, a cationic lipid is Compound 516. In embodiments, a cationic lipid is Compound 517. In embodiments, a cationic lipid is Compound 518. In embodiments, a cationic lipid is Compound 519. In embodiments, a cationic lipid is Compound 520. In embodiments, a cationic lipid is Compound 521. In embodiments, a cationic lipid is Compound 522. In embodiments, a cationic lipid is Compound 523. In embodiments, a cationic lipid is Compound 524. In embodiments, a cationic lipid is Compound 525. In embodiments, a cationic lipid is Compound 526. In embodiments, a cationic lipid is Compound 527. In embodiments, a cationic lipid is Compound 528. In embodiments, a cationic lipid is Compound 529. In embodiments, a cationic lipid is Compound 530. In embodiments, a cationic lipid is Compound 531. In embodiments, a cationic lipid is Compound 532. In embodiments, a cationic lipid is Compound 533. In embodiments, a cationic lipid is Compound 534. In embodiments, a cationic lipid is Compound 535. In embodiments, a cationic lipid is Compound 536. In embodiments, a cationic lipid is Compound 537. In embodiments, a cationic lipid is Compound 538. In embodiments, a cationic lipid is Compound 539. In embodiments, a cationic lipid is Compound 540. In embodiments, a cationic lipid is Compound 541. In embodiments, a cationic lipid is Compound 542. In embodiments, a cationic lipid is Compound 543. In embodiments, a cationic lipid is Compound 544. In embodiments, a cationic lipid is Compound 545. In embodiments, a cationic lipid is Compound 546. In embodiments, a cationic lipid is Compound 547. In embodiments, a cationic lipid is Compound 548. In embodiments, a cationic lipid is Compound 549. In embodiments, a cationic lipid is Compound 550. In embodiments, a cationic lipid is Compound 551. In embodiments, a cationic lipid is Compound 552.

Synthesis of Compounds of the Invention

The compounds described herein (e.g., a compound of Formula (A′), (A), (I), (I-a), (I-a′), (I-b), (I-b′), (I-c), (I-c′), (I-c-1), (I-c′-1), (I-c-2), (I-c′-2), (I-d), (I-d′), (I-d-1), (I-d-2), (I-e), (I-e′), (I-e-1), (I-e-2), (I-f), (I-f′), (II), (II-a), (II-a′), (III), (III′), (III-a), (III-a′), (III-b), (III-b′), (III-c), (III-c-1), (III-c′-1), (III-c-2), (III-c′-2), (III-c′), (III-d), (III-d′), (III-d-1), (III-d-2), (III-e), (III-e′), (III-e-1), (III-e-2), (III-f), (III-f′), (IV), (IV-a), or (IV-a′) such as any of Compounds 1-552) can be prepared according to methods known in the art, including the exemplary synthetic Scheme 1 provided herein.

For example, thioester compounds described herein (e.g., a compound as described in Table A or Table C) can be prepared as shown in Scheme A, where R³ and n can be any group or value as described herein. For example, a cyclic di-amino acid such as cyclic di(aspartic acid) (cDD) or cyclic di(glutamic acid) (cEE) with an appropriate thiol can provide the desired cationic lipid. Exemplary lipids prepared according to Scheme A are described in the Examples herein.

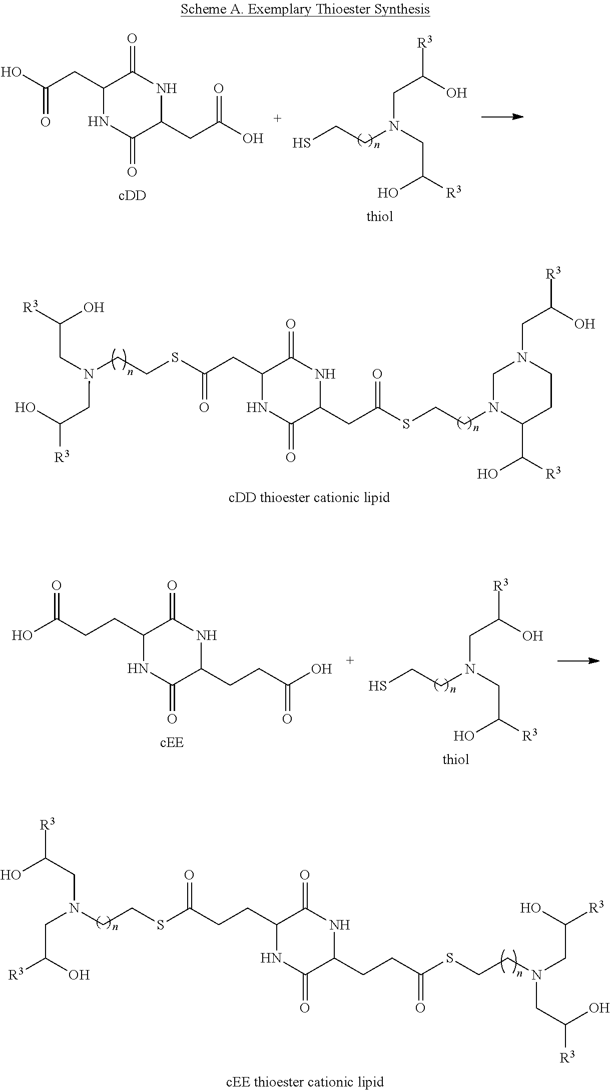

A further exemplary synthesis of thioester lipids described herein is shown in Scheme B, where R³ can be any group described herein. For example, starting di(amino acid) cEE can be activated using EDCl to form the succinimide ester cEE-OSu which can then be treated under basic conditions (e.g., Hunig's base or DMAP in DMF) to form the desired cationic lipid.

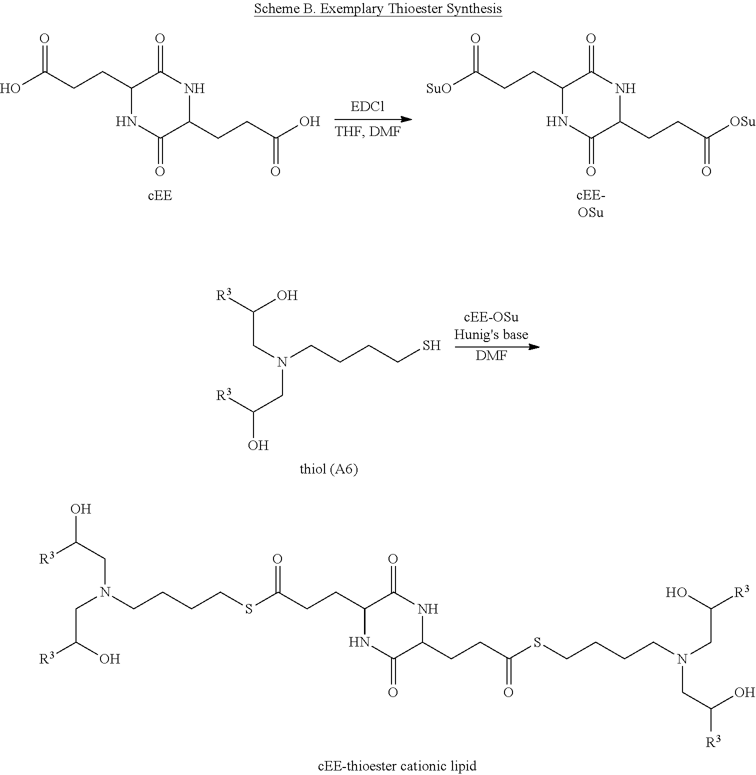

An exemplary synthesis of ester lipids described herein (e.g., a compound as described in Table B or Table D) is shown in Scheme C, where R³ and n can be any group or value as described herein. For example, a starting di(amino acid) cDD or cEE can be treated with a protected alcohol (e.g., a silylated alcohol such as alcohol A5) to form the protected form of the desired ester cationic lipid. Deprotection (e.g., of the silyl groups) can then afford the desired ester cationic lipid. This scheme also can be used to prepare thioesters as described herein by replacing the protected alcohol with a protected thiol (e.g., a silylated thiol)

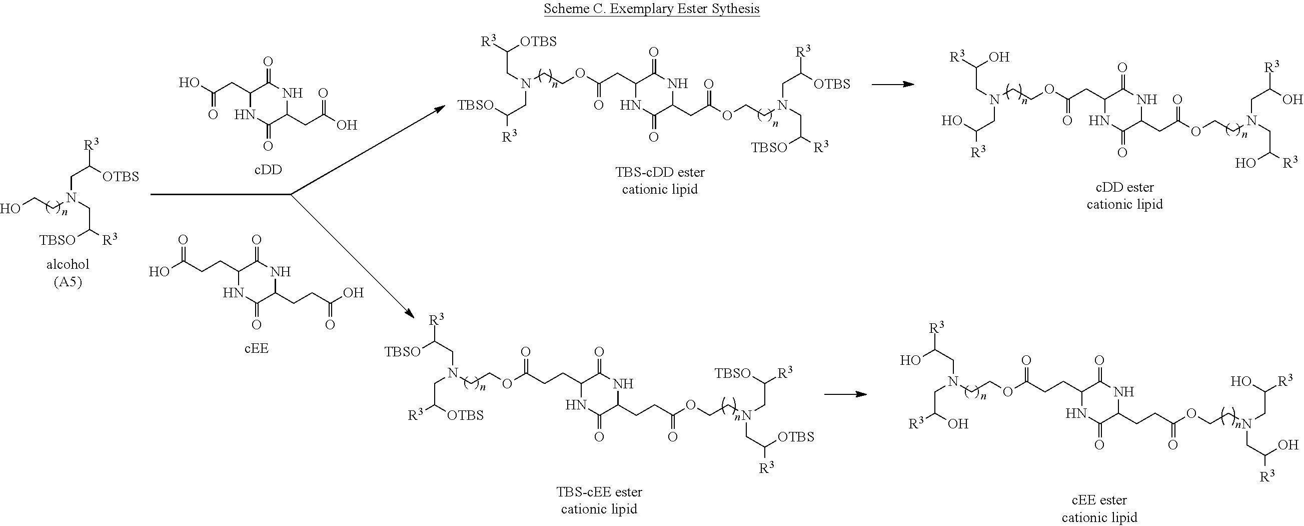

Homoserine-based lipids (e.g., a compound of Table E) can be prepared according to Scheme D, where R³ and n can be any group or value as described herein. For example, cyclic di-homoserine (cHse) can be esterified with a protected carboxylic acid to afford a silylated cHse cationic lipid intermediate. Deprotection of the silyl groups can then afford the desired cHse cationic lipid.

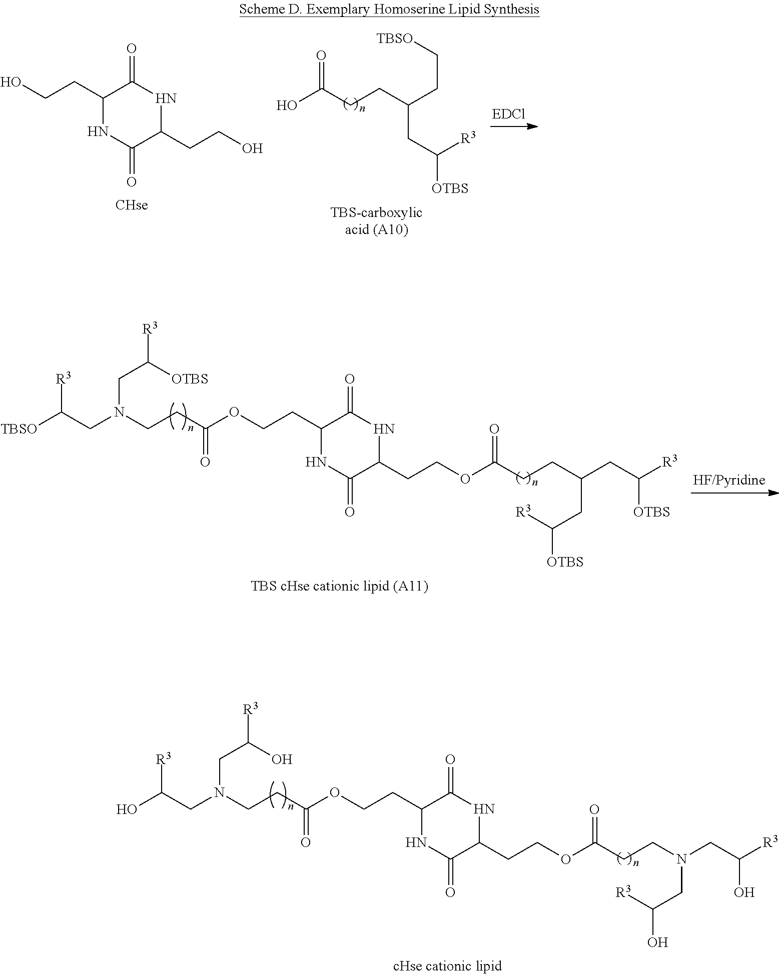

Nucleic Acids

The compounds described herein (e.g., a compound of Formula (A′), (A), (I), (I-a), (I-a′), (I-b), (I-b′), (I-c), (I-c′), (I-c-1), (I-c′-1), (I-c-2), (I-c′-2), (I-d), (I-d′), (I-d-1), (I-d-2), (I-e), (I-e′), (I-e-1), (I-e-2), (I-f), (I-f′), (II), (II-a), (II-a′), (III), (III′), (III-a), (III-a′), (III-b), (III-b′), (III-c), (III-c-1), (III-c′-1), (III-c-2), (III-c′-2), (III-c′), (III-d), (III-d′), (III-d-1), (III-d-2), (III-e), (III-e′), (III-e-1), (III-e-2), (III-f), (III-f′), (IV), (IV-a), or (IV-a′) such as any of Compounds 1-552) can be used to prepare compositions useful for the delivery of nucleic acids.

Synthesis of Nucleic Acids

Nucleic acids according to the present invention may be synthesized according to any known methods. For example, mRNAs according to the present invention may be synthesized via in vitro transcription (IVT). Briefly, IVT is typically performed with a linear or circular DNA template containing a promoter, a pool of ribonucleotide triphosphates, a buffer system that may include DTT and magnesium ions, and an appropriate RNA polymerase (e.g., T3, T7, mutated T7 or SP6 RNA polymerase), DNAse I, pyrophosphatase, and/or RNAse inhibitor. The exact conditions will vary according to the specific application.

In some embodiments, for the preparation of mRNA according to the invention, a DNA template is transcribed in vitro. A suitable DNA template typically has a promoter, for example a T3, T7, mutated T7 or SP6 promoter, for in vitro transcription, followed by desired nucleotide sequence for desired mRNA and a termination signal.

Desired mRNA sequence(s) according to the invention may be determined and incorporated into a DNA template using standard methods. For example, starting from a desired amino acid sequence (e.g., an enzyme sequence), a virtual reverse translation is carried out based on the degenerated genetic code. Optimization algorithms may then be used for selection of suitable codons. Typically, the G/C content can be optimized to achieve the highest possible G/C content on one hand, taking into the best possible account the frequency of the tRNAs according to codon usage on the other hand. The optimized RNA sequence can be established and displayed, for example, with the aid of an appropriate display device and compared with the original (wild-type) sequence. A secondary structure can also be analyzed to calculate stabilizing and destabilizing properties or, respectively, regions of the RNA.

As described above, the term “nucleic acid,” in its broadest sense, refers to any compound and/or substance that is or can be incorporated into a polynucleotide chain. DNA may be in the form of antisense DNA, plasmid DNA, parts of a plasmid DNA, pre-condensed DNA, a product of a polymerase chain reaction (PCR), vectors (e.g., P1, PAC, BAC, YAC, artificial chromosomes), expression cassettes, chimeric sequences, chromosomal DNA, or derivatives of these groups. RNA may be in the form of messenger RNA (mRNA), ribosomal RNA (rRNA), signal recognition particle RNA (7 SL RNA or SRP RNA), transfer RNA (tRNA), transfer-messenger RNA (tmRNA), small nuclear RNA (snRNA), small nucleolar RNA (snoRNA), SmY RNA, small Cajal body-specific RNA (scaRNA), guide RNA (gRNA), ribonuclease P (RNase P), Y RNA, telomerase RNA component (TERC), spliced leader RNA (SL RNA), antisense RNA (aRNA or asRNA), cis-natural antisense transcript (cis-NAT), CRISPR RNA (crRNA), long noncoding RNA (IncRNA), microRNA (miRNA), piwi-interacting RNA (piRNA), small interfering RNA (siRNA), transacting siRNA (tasiRNA), repeat associated siRNA (rasiRNA), 73K RNA, retrotransposons, a viral genome, a viroid, satellite RNA, or derivatives of these groups. In some embodiments, a nucleic acid is a mRNA encoding a protein.

Synthesis of mRNA

mRNAs according to the present invention may be synthesized according to any of a variety of known methods. For example, mRNAs according to the present invention may be synthesized via in vitro transcription (IVT). Briefly, IVT is typically performed with a linear or circular DNA template containing a promoter, a pool of ribonucleotide triphosphates, a buffer system that may include DTT and magnesium ions, and an appropriate RNA polymerase (e.g., T3, T7 or SP6 RNA polymerase), DNAse I, pyrophosphatase, and/or RNAse inhibitor. The exact conditions will vary according to the specific application. The exact conditions will vary according to the specific application. The presence of these reagents is undesirable in the final product according to several embodiments and may thus be referred to as impurities and a preparation containing one or more of these impurities may be referred to as an impure preparation. In some embodiments, the in vitro transcribing occurs in a single batch.

In some embodiments, for the preparation of mRNA according to the invention, a DNA template is transcribed in vitro. A suitable DNA template typically has a promoter, for example a T3, T7 or SP6 promoter, for in vitro transcription, followed by desired nucleotide sequence for desired mRNA and a termination signal.

Desired mRNA sequence(s) according to the invention may be determined and incorporated into a DNA template using standard methods. For example, starting from a desired amino acid sequence (e.g., an enzyme sequence), a virtual reverse translation is carried out based on the degenerated genetic code. Optimization algorithms may then be used for selection of suitable codons. Typically, the G/C content can be optimized to achieve the highest possible G/C content on one hand, taking into the best possible account the frequency of the tRNAs according to codon usage on the other hand. The optimized RNA sequence can be established and displayed, for example, with the aid of an appropriate display device and compared with the original (wild-type) sequence. A secondary structure can also be analyzed to calculate stabilizing and destabilizing properties or, respectively, regions of the RNA.

Modified mRNA

In some embodiments, mRNA according to the present invention may be synthesized as unmodified or modified mRNA. In some embodiments, an mRNA according to the invention comprises or consists of naturally-occurring nucleosides (or unmodified nucleosides; i.e., adenosine, guanosine, cytidine, and uridine). In other embodiments, an mRNA according to the present invention comprises nucleotide modifications in the RNA. A modified mRNA according to the invention can include nucleotide modification that are, for example, backbone modifications, sugar modifications or base modifications. In some embodiments, mRNAs may be synthesized from naturally occurring nucleotides and/or nucleotide analogs (modified nucleotides) including, but not limited to, purines (adenine (A), guanine (G)) or pyrimidines (thymine (T), cytosine (C), uracil (U)), and as modified nucleotides analogues or derivatives of purines and pyrimidines. In some embodiments, an mRNA according to the invention comprises one or more nucleoside analogs (e.g. adenosine analog, guanosine analog, cytidine analog, or uridine analog). In some embodiments, an mRNA comprises both unmodified and modified nucleosides. In some embodiments, the one or more nucleoside analogues include 1-methyl-adenine, 2-methyl-adenine, 2-methylthio-N-6-isopentenyl-adenine, N6-methyl-adenine, N6-isopentenyl-adenine, 2-thio-cytosine, 3-methyl-cytosine, 4-acetyl-cytosine, 5-methyl-cytosine, 2,6-diaminopurine, 1-methyl-guanine, 2-methyl-guanine, 2,2-dimethyl-guanine, 7-methyl-guanine, inosine, 1-methyl-inosine, pseudouracil (5-uracil), dihydro-uracil, 2-thio-uracil, 4-thio-uracil, 5-carboxymethylaminomethyl-2-thio-uracil, 5-(carboxyhydroxymethyl)-uracil, 5-fluoro-uracil, 5-bromo-uracil, 5-carboxymethylaminomethyl-uracil, 5-methyl-2-thio-uracil, 5-methyl-uracil, N-uracil-5-oxyacetic acid methyl ester, 5-methylaminomethyl-uracil, 5-methoxyaminomethyl-2-thio-uracil, 5′-methoxycarbonylmethyl-uracil, 5-methoxy-uracil, uracil-5-oxyacetic acid methyl ester, uracil-5-oxyacetic acid (v), 1-methyl-pseudouracil, queosine, beta.-D-mannosyl-queosine, wybutoxosine, and phosphoramidates, phosphorothioates, peptide nucleotides, methylphosphonates, 7-deazaguanosine, 5-methylcytosine and inosine. The preparation of such analogues is known to a person skilled in the art e.g., from the U.S. Pat. Nos. 4,373,071, 4,401,796, 4,415,732, 4,458,066, 4,500,707, 4,668,777, 4,973,679, 5,047,524, 5,132,418, 5,153,319, 5,262,530 and 5,700,642, the disclosures of which are incorporated by reference in their entirety.

In some embodiments, mRNAs may contain RNA backbone modifications. Typically, a backbone modification is a modification in which the phosphates of the backbone of the nucleotides contained in the RNA are modified chemically. Exemplary backbone modifications typically include, but are not limited to, modifications from the group consisting of methylphosphonates, methylphosphoramidates, phosphoramidates, phosphorothioates (e.g. cytidine 5′-O-(1-thiophosphate)), boranophosphates, positively charged guanidinium groups etc., which means by replacing the phosphodiester linkage by other anionic, cationic or neutral groups.

In some embodiments, mRNAs may contain sugar modifications. A typical sugar modification is a chemical modification of the sugar of the nucleotides it contains including, but not limited to, sugar modifications chosen from the group consisting of 4′-thio-ribonucleotide (see, e.g., US Patent Application Publication No. US 2016/0031928, incorporated by reference herein), 2′-deoxy-2′-fluoro-oligoribonucleotide (2′-fluoro-2′-deoxycytidine 5′-triphosphate, 2′-fluoro-2′-deoxyuridine 5′-triphosphate), 2′-deoxy-2′-deamine-oligoribonucleotide (2′-amino-2′-deoxycytidine 5′-triphosphate, 2′-amino-2′-deoxyuridine 5′-triphosphate), 2′-O-alkyloligoribonucleotide, 2′-deoxy-2′-C-alkyloligoribonucleotide (2′-O-methylcytidine 5′-triphosphate, 2′-methyluridine 5′-triphosphate), 2′-C-alkyloligoribonucleotide, and isomers thereof (2′-aracytidine 5′-triphosphate, 2′-arauridine 5′-triphosphate), or azidotriphosphates (2′-azido-2′-deoxycytidine 5′-triphosphate, 2′-azido-2′-deoxyuridine 5′-triphosphate).

In some embodiments, mRNAs may contain modifications of the bases of the nucleotides (base modifications). A modified nucleotide which contains a base modification is also called a base-modified nucleotide. Examples of such base-modified nucleotides include, but are not limited to, 2-amino-6-chloropurine riboside 5′-triphosphate, 2-aminoadenosine 5′-triphosphate, 2-thiocytidine 5′-triphosphate, 2-thiouridine 5′-triphosphate, 4-thiouridine 5′-triphosphate, 5-aminoallylcytidine 5′-triphosphate, 5-aminoallyluridine 5′-triphosphate, 5-bromocytidine 5′-triphosphate, 5-bromouridine 5′-triphosphate, 5-iodocytidine 5′-triphosphate, 5-iodouridine 5′-triphosphate, 5-methylcytidine 5′-triphosphate, 5-methyluridine 5′-triphosphate, 6-azacytidine 5′-triphosphate, 6-azauridine 5′-triphosphate, 6-chloropurine riboside 5′-triphosphate, 7-deazaadenosine 5′-triphosphate, 7-deazaguanosine 5′-triphosphate, 8-azaadenosine 5′-triphosphate, 8-azidoadenosine 5′-triphosphate, benzimidazole riboside 5′-triphosphate, N1-methyladenosine 5′-triphosphate, N1-methylguanosine 5′-triphosphate, N6-methyladenosine 5′-triphosphate, 06-methylguanosine 5′-triphosphate, pseudouridine 5′-triphosphate, puromycin 5′-triphosphate or xanthosine 5′-triphosphate.

Typically, mRNA synthesis includes the addition of a “cap” on the N-terminal (5′) end, and a “tail” on the C-terminal (3′) end. The presence of the cap is important in providing resistance to nucleases found in most eukaryotic cells. The presence of a “tail” serves to protect the mRNA from exonuclease degradation.

Thus, in some embodiments, mRNAs include a 5′ cap structure. A 5′ cap is typically added as follows: first, an RNA terminal phosphatase removes one of the terminal phosphate groups from the 5′ nucleotide, leaving two terminal phosphates; guanosine triphosphate (GTP) is then added to the terminal phosphates via a guanylyl transferase, producing a 5′5′5 triphosphate linkage; and the 7-nitrogen of guanine is then methylated by a methyltransferase. Examples of cap structures include, but are not limited to, m7G(5′)ppp (5′(A,G(5′)ppp(5′)A and G(5′)ppp(5′)G.

In some embodiments, mRNAs include a 3′ poly(A) tail structure. A poly-A tail on the 3′ terminus of mRNA typically includes about 10 to 300 adenosine nucleotides (e.g., about 10 to 200 adenosine nucleotides, about 10 to 150 adenosine nucleotides, about 10 to 100 adenosine nucleotides, about 20 to 70 adenosine nucleotides, or about 20 to 60 adenosine nucleotides). In some embodiments, mRNAs include a 3′ poly(C) tail structure. A suitable poly-C tail on the 3′ terminus of mRNA typically include about 10 to 200 cytosine nucleotides (e.g., about 10 to 150 cytosine nucleotides, about 10 to 100 cytosine nucleotides, about 20 to 70 cytosine nucleotides, about 20 to 60 cytosine nucleotides, or about 10 to 40 cytosine nucleotides). The poly-C tail may be added to the poly-A tail or may substitute the poly-A tail.

In some embodiments, mRNAs include a 5′ and/or 3′ untranslated region. In some embodiments, a 5′ untranslated region includes one or more elements that affect an mRNA's stability or translation, for example, an iron responsive element. In some embodiments, a 5′ untranslated region may be between about 50 and 500 nucleotides in length.

In some embodiments, a 3′ untranslated region includes one or more of a polyadenylation signal, a binding site for proteins that affect an mRNA's stability of location in a cell, or one or more binding sites for miRNAs. In some embodiments, a 3′ untranslated region may be between 50 and 500 nucleotides in length or longer.

Cap Structure

In some embodiments, mRNAs include a 5′ cap structure. A 5′ cap is typically added as follows: first, an RNA terminal phosphatase removes one of the terminal phosphate groups from the 5′ nucleotide, leaving two terminal phosphates; guanosine triphosphate (GTP) is then added to the terminal phosphates via a guanylyl transferase, producing a 5′5′5 triphosphate linkage; and the 7-nitrogen of guanine is then methylated by a methyltransferase. Examples of cap structures include, but are not limited to, m7G(5′)ppp (5′(A,G(5′)ppp(5′)A and G(5′)ppp(5′)G.

Naturally occurring cap structures comprise a 7-methyl guanosine that is linked via a triphosphate bridge to the 5′-end of the first transcribed nucleotide, resulting in a dinucleotide cap of m⁷G(5′)ppp(5′)N, where N is any nucleoside. In vivo, the cap is added enzymatically. The cap is added in the nucleus and is catalyzed by the enzyme guanylyl transferase. The addition of the cap to the 5′ terminal end of RNA occurs immediately after initiation of transcription. The terminal nucleoside is typically a guanosine, and is in the reverse orientation to all the other nucleotides, i.e., G(5′)ppp(5′)GpNpNp.

A common cap for mRNA produced by in vitro transcription is m⁷G(5′)ppp(5′)G, which has been used as the dinucleotide cap in transcription with T7 or SP6 RNA polymerase in vitro to obtain RNAs having a cap structure in their 5′-termini. The prevailing method for the in vitro synthesis of caPPEd mRNA employs a pre-formed dinucleotide of the form m⁷G(5′)ppp(5′)G (“m⁷GpppG”) as an initiator of transcription.

To date, a usual form of a synthetic dinucleotide cap used in in vitro translation experiments is the Anti-Reverse Cap Analog (“ARCA”) or modified ARCA, which is generally a modified cap analog in which the 2′ or 3′ OH group is replaced with —OCH₃.

Additional cap analogs include, but are not limited to, a chemical structures selected from the group consisting of m⁷GpppG, m⁷GpppA, m⁷GpppC; unmethylated cap analogs (e.g., GpppG); dimethylated cap analog (e.g., m^{2,7}GpppG), trimethylated cap analog (e.g., m^{2,2,7}GpppG), dimethylated symmetrical cap analogs (e.g., m⁷Gpppm⁷G), or anti reverse cap analogs (e.g., ARCA; m^{7,2′Ome}GpppG, m^{72′d}GpppG, m^{7,3′Ome}GpppG, m^{7,3′d}GpppG and their tetraphosphate derivatives) (see, e.g., Jemielity, J. et al., “Novel ‘anti-reverse’ cap analogs with superior translational properties”, RNA, 9: 1108-1122 (2003)).

In some embodiments, a suitable cap is a 7-methyl guanylate (“m⁷G”) linked via a triphosphate bridge to the 5′-end of the first transcribed nucleotide, resulting in m⁷G(5′)ppp(5′)N, where N is any nucleoside. A preferred embodiment of a m⁷G cap utilized in embodiments of the invention is m⁷G(5′)ppp(5′)G.

In some embodiments, the cap is a Cap0 structure. Cap0 structures lack a 2′-O-methyl residue of the ribose attached to bases 1 and 2. In some embodiments, the cap is a Cap1 structure. Cap1 structures have a 2′-O-methyl residue at base 2. In some embodiments, the cap is a Cap2 structure. Cap2 structures have a 2′-O-methyl residue attached to both bases 2 and 3.

A variety of m⁷G cap analogs are known in the art, many of which are commercially available. These include the m⁷GpppG described above, as well as the ARCA 3′-OCH₃ and 2′-OCH₃ cap analogs (Jemielity, J. et al., RNA, 9: 1108-1122 (2003)). Additional cap analogs for use in embodiments of the invention include N7-benzylated dinucleoside tetraphosphate analogs (described in Grudzien, E. et al., RNA, 10: 1479-1487 (2004)), phosphorothioate cap analogs (described in Grudzien-Nogalska, E., et al., RNA, 13: 1745-1755 (2007)), and cap analogs (including biotinylated cap analogs) described in U.S. Pat. Nos. 8,093,367 and 8,304,529, incorporated by reference herein.

Tail Structure

Typically, the presence of a “tail” serves to protect the mRNA from exonuclease degradation. The poly A tail is thought to stabilize natural messengers and synthetic sense RNA. Therefore, in certain embodiments a long poly A tail can be added to an mRNA molecule thus rendering the RNA more stable. Poly A tails can be added using a variety of art-recognized techniques. For example, long poly A tails can be added to synthetic or in vitro transcribed RNA using poly A polymerase (Yokoe, et al. Nature Biotechnology. 1996; 14: 1252-1256). A transcription vector can also encode long poly A tails. In addition, poly A tails can be added by transcription directly from PCR products. Poly A may also be ligated to the 3′ end of a sense RNA with RNA ligase (see, e.g., Molecular Cloning A Laboratory Manual, 2nd Ed., ed. by Sambrook, Fritsch and Maniatis (Cold Spring Harbor Laboratory Press: 1991 edition)).

In some embodiments, mRNAs include a 3′ poly(A) tail structure. Typically, the length of the poly A tail can be at least about 10, 50, 100, 200, 300, 400 at least 500 nucleotides. In some embodiments, a poly-A tail on the 3′ terminus of mRNA typically includes about 10 to 300 adenosine nucleotides (e.g., about 10 to 200 adenosine nucleotides, about 10 to 150 adenosine nucleotides, about 10 to 100 adenosine nucleotides, about 20 to 70 adenosine nucleotides, or about 20 to 60 adenosine nucleotides). In some embodiments, mRNAs include a 3′ poly(C) tail structure. A suitable poly-C tail on the 3′ terminus of mRNA typically include about 10 to 200 cytosine nucleotides (e.g., about 10 to 150 cytosine nucleotides, about 10 to 100 cytosine nucleotides, about 20 to 70 cytosine nucleotides, about 20 to 60 cytosine nucleotides, or about 10 to 40 cytosine nucleotides). The poly-C tail may be added to the poly-A tail or may substitute the poly-A tail.

In some embodiments, the length of the poly A or poly C tail is adjusted to control the stability of a modified sense mRNA molecule of the invention and, thus, the transcription of protein. For example, since the length of the poly A tail can influence the half-life of a sense mRNA molecule, the length of the poly A tail can be adjusted to modify the level of resistance of the mRNA to nucleases and thereby control the time course of polynucleotide expression and/or polypeptide production in a target cell.

5′ and 3′ Untranslated Region

In some embodiments, mRNAs include a 5′ and/or 3′ untranslated region. In some embodiments, a 5′ untranslated region includes one or more elements that affect an mRNA's stability or translation, for example, an iron responsive element. In some embodiments, a 5′ untranslated region may be between about 50 and 500 nucleotides in length.

In some embodiments, a 3′ untranslated region includes one or more of a polyadenylation signal, a binding site for proteins that affect an mRNA's stability of location in a cell, or one or more binding sites for miRNAs. In some embodiments, a 3′ untranslated region may be between 50 and 500 nucleotides in length or longer.

Exemplary 3′ and/or 5′ UTR sequences can be derived from mRNA molecules which are stable (e.g., globin, actin, GAPDH, tubulin, histone, or citric acid cycle enzymes) to increase the stability of the sense mRNA molecule. For example, a 5′ UTR sequence may include a partial sequence of a CMV immediate-early 1 (IE1) gene, or a fragment thereof to improve the nuclease resistance and/or improve the half-life of the polynucleotide. Also contemplated is the inclusion of a sequence encoding human growth hormone (hGH), or a fragment thereof to the 3′ end or untranslated region of the polynucleotide (e.g., mRNA) to further stabilize the polynucleotide. Generally, these modifications improve the stability and/or pharmacokinetic properties (e.g., half-life) of the polynucleotide relative to their unmodified counterparts, and include, for example modifications made to improve such polynucleotides' resistance to in vivo nuclease digestion.

### Pharmaceutical Formulations of Cationic Lipids and Nucleic Acids

In certain embodiments, the compounds described herein (e.g., a compound of Formula (A′), (A), (I), (I-a), (I-a′), (I-b), (I-b′), (I-c), (I-c′), (I-c-1), (I-c′-1), (I-c-2), (I-c′-2), (I-d), (I-d′), (I-d-1), (I-d-2), (I-e), (I-e′), (I-e-1), (I-e-2), (I-f), (I-f′) (II), (II-a), (II-a′), (III), (III′), (III-a), (III-a′), (III-b), (III-b′), (III-c), (III-c-1), (III-c′-1), (III-c-2), (III-c′-2), (III-c′), (III-d), (III-d′), (III-d-1), (III-d-2), (III-e), (III-e′), (III-e-1), (III-e-2), (III-f), (III-f′), (IV), (IV-a), or (IV-a′) such as any of Compounds 1-552), as well as pharmaceutical and liposomal compositions comprising such lipids, can be used in formulations to facilitate the delivery of encapsulated materials (e.g., one or more polynucleotides such as mRNA) to, and subsequent transfection of one or more target cells. For example, in certain embodiments cationic lipids described herein (and compositions such as liposomal compositions comprising such lipids) are characterized as resulting in one or more of receptor-mediated endocytosis, clathrin-mediated and caveolae-mediated endocytosis, phagocytosis and macropinocytosis, fusogenicity, endosomal or lysosomal disruption and/or releasable properties that afford such compounds advantages relative other similarly classified lipids.

According to the present invention, a nucleic acid, e.g., mRNA encoding a protein (e.g., a full length, fragment or portion of a protein) as described herein may be delivered via a delivery vehicle comprising a compound as described herein (e.g., a compound of Formula (A′), (A), (I), (I-a), (I-a′), (I-b), (I-b′), (I-c), (I-c′), (I-c-1), (I-c′-1), (I-c-2), (I-c′-2), (I-d), (I-d′), (I-d-1), (I-d-2), (I-e), (I-e′), (I-e-1), (I-e-2), (I-f), (I-f′), (II), (II-a), (II-a′), (III), (III′), (III-a), (III-a′), (III-b), (III-b′), (III-c), (III-c-1), (III-c′-1), (III-c-2), (III-c′-2), (III-c′), (III-d), (III-d′), (III-d-1), (III-d-2), (III-e), (III-e′), (III-e-1), (III-e-2), (III-f), (III-f′), (IV), (IV-a), or (IV-a′) such as any of Compounds 1-552).

As used herein, the terms “delivery vehicle,” “transfer vehicle,” “nanoparticle” or grammatical equivalent, are used interchangeably.

For example, the present invention provides a composition (e.g., a pharmaceutical composition) comprising a compound described herein (e.g., a compound of Formula (A′), (A), (I), (I-a), (I-a′), (I-b), (I-b′), (I-c), (I-c′), (I-c-1), (I-c′-1), (I-c-2), (I-c′-2), (I-d), (I-d′), (I-d-1), (I-d-2), (I-e), (I-e′), (I-e-1), (I-e-2), (I-f), (I-f′), (II), (II-a), (II-a′), (III), (III′), (III-a), (III-a′), (III-b), (III-b′), (III-c), (III-c-1), (III-c′-1), (III-c-2), (III-c′-2), (III-c′), (III-d), (III-d′), (III-d-1), (III-d-2), (III-e), (III-e′), (III-e-1), (III-e-2), (III-f), (III-f′), (IV), (IV-a), or (IV-a′) such as any of Compounds 1-552) and one or more polynucleotides. A composition (e.g., a pharmaceutical composition) may further comprise one or more cationic lipids, one or more non-cationic lipids, one or more cholesterol-based lipids and/or one or more PEG-modified lipids.

In certain embodiments a composition exhibits an enhanced (e.g., increased) ability to transfect one or more target cells. Accordingly, also provided herein are methods of transfecting one or more target cells. Such methods generally comprise the step of contacting the one or more target cells with the cationic lipids and/or pharmaceutical compositions disclosed herein (e.g., a liposomal formulation comprising a compound described herein (e.g., a compound of Formula (A′), (A), (I), (I-a), (I-a′), (I-b), (I-b′), (I-c), (I-c′), (I-c-1), (I-c′-1), (I-c-2), (I-c′-2), (I-d), (I-d′), (I-d-1), (I-d-2), (I-e), (I-e′), (I-e-1), (I-e-2), (I-f), (I-f′), (II), (II-a), (II-a′), (III), (III′), (III-a), (III-a′), (III-b), (III-b′), (III-c), (III-c-1), (III-c′-1), (III-c-2), (III-c′-2), (III-c′), (III-d), (III-d′), (III-d-1), (III-d-2), (III-e), (III-e′), (III-e-1), (III-e-2), (III-f), (III-f′), (IV), (IV-a), or (IV-a′) such as any of Compounds 1-552) encapsulating one or more polynucleotides) such that the one or more target cells are transfected with the materials encapsulated therein (e.g., one or more polynucleotides). As used herein, the terms “transfect” or “transfection” refer to the intracellular introduction of one or more encapsulated materials (e.g., nucleic acids and/or polynucleotides) into a cell, or preferably into a target cell. The introduced polynucleotide may be stably or transiently maintained in the target cell. The term “transfection efficiency” refers to the relative amount of such encapsulated material (e.g., polynucleotides) up-taken by, introduced into and/or expressed by the target cell which is subject to transfection. In practice, transfection efficiency may be estimated by the amount of a reporter polynucleotide product produced by the target cells following transfection. In certain embodiments, the compounds and pharmaceutical compositions described herein demonstrate high transfection efficiencies thereby improving the likelihood that appropriate dosages of the encapsulated materials (e.g., one or more polynucleotides) will be delivered to the site of pathology and subsequently expressed, while at the same time minimizing potential systemic adverse effects or toxicity associated with the compound or their encapsulated contents.

Following transfection of one or more target cells by, for example, the polynucleotides encapsulated in the one or more lipid nanoparticles comprising the pharmaceutical or liposomal compositions disclosed herein, the production of the product (e.g., a polypeptide or protein) encoded by such polynucleotide may be preferably stimulated and the capability of such target cells to express the polynucleotide and produce, for example, a polypeptide or protein of interest is enhanced. For example, transfection of a target cell by one or more compounds or pharmaceutical compositions encapsulating mRNA will enhance (i.e., increase) the production of the protein or enzyme encoded by such mRNA.

Further, delivery vehicles described herein (e.g., liposomal delivery vehicles) may be prepared to preferentially distribute to other target tissues, cells or organs, such as the heart, lungs, kidneys, spleen. In embodiments, the lipid nanoparticles of the present invention may be prepared to achieve enhanced delivery to the target cells and tissues. For example, polynucleotides (e.g., mRNA) encapsulated in one or more of the compounds or pharmaceutical and liposomal compositions described herein can be delivered to and/or transfect targeted cells or tissues. In some embodiments, the encapsulated polynucleotides (e.g., mRNA) are capable of being expressed and functional polypeptide products produced (and in some instances excreted) by the target cell, thereby conferring a beneficial property to, for example the target cells or tissues. Such encapsulated polynucleotides (e.g., mRNA) may encode, for example, a hormone, enzyme, receptor, polypeptide, peptide or other protein of interest.

Liposomal Delivery Vehicles

In some embodiments, a composition is a suitable delivery vehicle. In embodiments, a composition is a liposomal delivery vehicle, e.g., a lipid nanoparticle.

Any embodiment (or any combination of any embodiments) described herein is suitable for use with any compound described herein (e.g., a compound of Formula (A′), (A), (I), (I-a), (I-a′), (I-b), (I-b′), (I-c), (I-c′), (I-c-1), (I-c′-1), (I-c-2), (I-c′-2), (I-d), (I-d′), (I-d-1), (I-d-2), (I-e), (I-e′), (I-e-1), (I-e-2), (I-f), (I-f′), (II), (II-a), (II-a′), (III), (III′), (III-a), (III-a′), (III-b), (III-b′), (III-c), (III-c-1), (III-c′-1), (III-c-2), (III-c′-2), (III-c′), (III-d), (III-d′), (III-d-1), (III-d-2), (III-e), (III-e′), (III-e-1), (III-e-2), (III-f), (III-f′), (IV), (IV-a), or (IV-a′) such as any of Compounds 1-552).

The terms “liposomal delivery vehicle” and “liposomal composition” are used interchangeably.

Enriching liposomal compositions with one or more of the cationic lipids disclosed herein may be used as a means of improving (e.g., reducing) the toxicity or otherwise conferring one or more desired properties to such enriched liposomal composition (e.g., improved delivery of the encapsulated polynucleotides to one or more target cells and/or reduced in vivo toxicity of a liposomal composition). Accordingly, also contemplated are pharmaceutical compositions, and in particular liposomal compositions, that comprise one or more of the cationic lipids disclosed herein.

Thus, in certain embodiments, the compounds described herein (e.g., a compound of Formula (A′), (A), (I), (I-a), (I-a′), (I-b), (I-b′), (I-c), (I-c′), (I-c-1), (I-c′-1), (I-c-2), (I-c′-2), (I-d), (I-d′), (I-d-1), (I-d-2), (I-e), (I-e′), (I-e-1), (I-e-2), (I-f), (I-f′), (II), (II-a), (II-a′), (III), (III′), (III-a), (III-a′), (III-b), (III-b′), (III-c), (III-c-1), (III-c′-1), (III-c-2), (III-c′-2), (III-c′), (III-d), (III-d′), (III-d-1), (III-d-2), (III-e), (III-e′), (III-e-1), (III-e-2), (III-f), (III-f′), (IV), (IV-a), or (IV-a′) such as any of Compounds 1-552) may be used as a component of a liposomal composition to facilitate or enhance the delivery and release of encapsulated materials (e.g., one or more therapeutic agents) to one or more target cells (e.g., by permeating or fusing with the lipid membranes of such target cells).

As used herein, liposomal delivery vehicles, e.g., lipid nanoparticles, are usually characterized as microscopic vesicles having an interior aqua space sequestered from an outer medium by a membrane of one or more bilayers. Bilayer membranes of liposomes are typically formed by amphiphilic molecules, such as lipids of synthetic or natural origin that comprise spatially separated hydrophilic and hydrophobic domains (Lasic, Trends Biotechnol., 16: 307-321, 1998). Bilayer membranes of the liposomes can also be formed by amphiphilic polymers and surfactants (e.g., polymerosomes, niosomes, etc.). In the context of the present invention, a liposomal delivery vehicle typically serves to transport a desired nucleic acid (e.g., mRNA or MCNA) to a target cell or tissue.

In certain embodiments, such compositions (e.g., liposomal compositions) are loaded with or otherwise encapsulate materials, such as for example, one or more biologically-active polynucleotides (e.g., mRNA).

In some embodiments, a nanoparticle delivery vehicle is a liposome. In some embodiments, a liposome comprises one or more cationic lipids, one or more non-cationic lipids, one or more cholesterol-based lipids, or one or more PEG-modified lipids. A typical liposome for use with the invention is composed of four lipid components: a cationic lipid, a non-cationic lipid (e.g., DOPE or DEPE), a cholesterol-based lipid (e.g., cholesterol) and a PEG-modified lipid (e.g., DMG-PEG2K).

In embodiments, a composition (e.g., a pharmaceutical composition) comprises an mRNA encoding a protein, encapsulated within a liposome. In embodiments, a liposome comprises one or more cationic lipids, one or more non-cationic lipids, one or more cholesterol-based lipids and one or more PEG-modified lipids, and wherein at least one cationic lipid is a compound as described herein (e.g., a compound of Formula (A′), (A), (I), (I-a), (I-a′), (I-b), (I-b′), (I-c), (I-c′), (I-c-1), (I-c′-1), (I-c-2), (I-c′-2), (I-d), (I-d′), (I-d-1), (I-d-2), (I-e), (I-e′), (I-e-1), (I-e-2), (I-f), (I-f′), (II), (II-a), (II-a′), (III), (III′), (III-a), (III-a′), (III-b), (III-b′), (III-c), (III-c-1), (III-c′-1), (III-c-2), (III-c′-2), (III-c′), (III-d), (III-d′), (III-d-1), (III-d-2), (III-e), (III-e′), (III-e-1), (III-e-2), (III-f), (III-f′), (IV), (IV-a), or (IV-a′) such as any of Compounds 1-552). In embodiments, a composition comprises an mRNA encoding for a protein (e.g., any protein described herein). In embodiments, a composition comprises an mRNA encoding for cystic fibrosis transmembrane conductance regulator (CFTR) protein. In embodiments, a composition comprises an mRNA encoding for ornithine transcarbamylase (OTC) protein.

In embodiments, a composition (e.g., a pharmaceutical composition) comprises a nucleic acid encapsulated within a liposome, wherein the liposome comprises any compound described herein (e.g., a compound of Formula (A′), (A), (I), (I-a), (I-a′), (I-b), (I-b′), (I-c), (I-c′), (I-c-1), (I-c′-1), (I-c-2), (I-c′-2), (I-d), (I-d′), (I-d-1), (I-d-2), (I-e), (I-e′), (I-e-1), (I-e-2), (I-f), (I-f′), (II), (II-a), (II-a′), (III), (III′), (III-a), (III-a′), (III-b), (III-b′), (III-c), (III-c-1), (III-c′-1), (III-c-2), (III-c′-2), (III-c′), (III-d), (III-d′), (III-d-1), (III-d-2), (III-e), (III-e′), (III-e-1), (III-e-2), (III-f), (III-f′), (IV), (IV-a), or (IV-a′) such as any of Compounds 1-552) as described herein.

In embodiments, a nucleic acid is an mRNA encoding a peptide or protein. In embodiments, an mRNA encodes a peptide or protein for use in the delivery to or treatment of the lung of a subject or a lung cell (e.g., an mRNA encodes cystic fibrosis transmembrane conductance regulator (CFTR) protein). In embodiments, an mRNA encodes a peptide or protein for use in the delivery to or treatment of the liver of a subject or a liver cell (e.g., an mRNA encodes ornithine transcarbamylase (OTC) protein). Still other exemplary mRNAs are described herein.

In embodiments, a liposomal delivery vehicle (e.g., a lipid nanoparticle) can have a net positive charge.

In embodiments, a liposomal delivery vehicle (e.g., a lipid nanoparticle) can have a net negative charge.

In embodiments, a liposomal delivery vehicle (e.g., a lipid nanoparticle) can have a net neutral charge.

In embodiments, a lipid nanoparticle that encapsulates a nucleic acid (e.g., mRNA encoding a peptide or protein) comprises one or more compounds described herein ((e.g., a compound of Formula (A′), (A), (I), (I-a), (I-a′), (I-b), (I-b′), (I-c), (I-c′), (I-c-1), (I-c′-1), (I-c-2), (I-c′-2), (I-d), (I-d′), (I-d-1), (I-d-2), (I-e), (I-e′), (I-e-1), (I-e-2), (I-f), (I-f′), (II), (II-a), (II-a′), (III), (III′), (III-a), (III-a′), (III-b), (III-b′), (III-c), (III-c-1), (III-c′-1), (III-c-2), (III-c′-2), (III-c′), (III-d), (III-d′), (III-d-1), (III-d-2), (III-e), (III-e′), (III-e-1), (III-e-2), (III-f), (III-f′), (IV), (IV-a), or (IV-a′) such as any of Compounds 1-552).

For example, the amount of a compound as described herein (e.g a compound of Formula (A′), (A), (I), (I-a), (I-a′), (I-b), (I-b′), (I-c), (I-c′), (I-c-1), (I-c′-1), (I-c-2), (I-c′-2), (I-d), (I-d′), (I-d-1), (I-d-2), (I-e), (I-e′), (I-e-1), (I-e-2), (I-f), (I-f′), (II), (II-a), (II-a′), (III), (III′), (III-a), (III-a′), (III-b), (III-b′), (III-c), (III-c-1), (III-c′-1), (III-c-2), (III-c′-2), (III-c′), (III-d), (III-d′), (III-d-1), (III-d-2), (III-e), (III-e′), (III-e-1), (III-e-2), (III-f), (III-f′), (IV), (IV-a), or (IV-a′) such as any of Compounds 1-552) in a composition can be described as a percentage (“wt %”) of the combined dry weight of all lipids of a composition (e.g., the combined dry weight of all lipids present in a liposomal composition).

In embodiments of the pharmaceutical compositions described herein, a compound as described herein (e.g., a compound of Formula (A′), (A), (I), (I-a), (I-a′), (I-b), (I-b′), (I-c), (I-c′), (I-c-1), (I-c′-1), (I-c-2), (I-c′-2), (I-d), (I-d′), (I-d-1), (I-d-2), (I-e), (I-e′), (I-e-1), (I-e-2), (I-f), (I-f′), (II), (II-a), (II-a′), (III), (III′), (III-a), (III-a′), (III-b), (III-b′), (III-c), (III-c-1), (III-c′-1), (III-c-2), (III-c′-2), (III-c′), (III-d), (III-d′), (III-d-1), (III-d-2), (III-e), (III-e′), (III-e-1), (III-e-2), (III-f), (III-f′), (IV), (IV-a), or (IV-a′) such as any of Compounds 1-552) is present in an amount that is about 0.5 wt % to about 30 wt % (e.g., about 0.5 wt % to about 50 wt % (e.g., about 0.5 wt % to about 20 wt %) of the combined dry weight of all lipids present in a composition (e.g., a liposomal composition).

In embodiments, a compound as described herein (e.g., a compound of Formula (A′), (A), (I), (I-a), (I-a′), (I-b), (I-b′), (I-c), (I-c′), (I-c-1), (I-c′-1), (I-c-2), (I-c′-2), (I-d), (I-d′), (I-d-1), (I-d-2), (I-e), (I-e′), (I-e-1), (I-e-2), (I-f), (I-f), (II), (II-a), (II-a′), (III), (III′), (III-a), (III-a′), (III-b), (III-b′), (III-c), (III-c-1), (III-c′-1), (III-c-2), (III-c′-2), (III-c′), (III-d), (III-d′), (III-d-1), (III-d-2), (III-e), (III-e′), (III-e-1), (III-e-2), (III-f), (III-f′), (IV), (IV-a), or (IV-a′) such as any of Compounds 1-552) is present in an amount that is about 1 wt % to about 50 wt %, about 1 wt % to about 40 wt %, about 1 wt % to about 30 wt %, about 1 wt % to about 20 wt %, about 1 wt % to about 15 wt %, about 1 wt % to about 10 wt %, about 5 wt % to about 25 wt %, about 10 wt % to about 30 wt %, or about 20 wt % to about 40 wt % of the combined dry weight of all lipids present in a composition (e.g., a liposomal composition). In embodiments, a compound as described herein (e.g., a compound of Formula (A′), (A), (I), (I-a), (I-a′), (I-b), (I-b′), (I-c), (I-c′), (I-c-1), (I-c′-1), (I-c-2), (I-c′-2), (I-d), (I-d′), (I-d-1), (I-d-2), (I-e), (I-e′), (I-e-1), (I-e-2), (I-f), (I-f′), (II), (II-a), (II-a′), (III), (III′), (III-a), (III-a′), (III-b), (III-b′), (III-c), (III-c-1), (III-c′-1), (III-c-2), (III-c′-2), (III-c′), (III-d), (III-d′), (III-d-1), (III-d-2), (III-e), (III-e′), (III-e-1), (III-e-2), (III-f), (III-f′), (IV), (IV-a), or (IV-a′) such as any of Compounds 1-552) is present in an amount that is about 0.5 wt % to about 5 wt %, about 1 wt % to about 10 wt %, about 5 wt % to about 20 wt %, or about 10 wt % to about 20 wt % of the combined molar amounts of all lipids present in a composition such as a liposomal delivery vehicle.

In embodiments, the amount of a compound as described herein (e.g., a compound of Formula (A′), (A), (I), (I-a), (I-a′), (I-b), (I-b′), (I-c), (I-c′), (I-c-1), (I-c′-1), (I-c-2), (I-c′-2), (I-d), (I-d′), (I-d-1), (I-d-2), (I-e), (I-e′), (I-e-1), (I-e-2), (I-f), (I-f′), (II), (II-a), (II-a′), (III), (III′), (III-a), (III-a′), (III-b), (III-b′), (III-c), (III-c-1), (III-c′-1), (III-c-2), (III-c′-2), (III-c′), (III-d), (III-d′), (III-d-1), (III-d-2), (III-e), (III-e′), (III-e-1), (III-e-2), (III-f), (III-f′), (IV), (IV-a), or (IV-a′) such as any of Compounds 1-552) is present in an amount that is at least about 5 wt %, about 10 wt %, about 15 wt %, about 20 wt %, about 25 wt %, about 30 wt %, about 35 wt %, about 40 wt %, about 45 wt %, about 50 wt %, about 55 wt %, about 60 wt %, about 65 wt %, about 70 wt %, about 75 wt %, about 80 wt %, about 85 wt %, about 90 wt %, about 95 wt %, about 96 wt %, about 97 wt %, about 98 wt %, or about 99 wt % of the combined dry weight of total lipids in a composition (e.g., a liposomal composition).

In embodiments, the amount of a compound as described herein ((e.g., a compound of Formula (A′), (A), (I), (I-a), (I-a′), (I-b), (I-b′), (I-c), (I-c′), (I-c-1), (I-c′-1), (I-c-2), (I-c′-2), (I-d), (I-d′), (I-d-1), (I-d-2), (I-e), (I-e′), (I-e-1), (I-e-2), (I-f), (I-f′), (II), (II-a), (II-a′), (III), (III′), (III-a), (III-a′), (III-b), (III-b′), (III-c), (III-c-1), (III-c′-1), (III-c-2), (III-c′-2), (III-c′), (III-d), (III-d′), (III-d-1), (III-d-2), (III-e), (III-e′), (III-e-1), (III-e-2), (III-f), (III-f′), (IV), (IV-a), or (IV-a′) such as any of Compounds 1-552) is present in an amount that is no more than about 5 wt %, about 10 wt %, about 15 wt %, about 20 wt %, about 25 wt %, about 30 wt %, about 35 wt %, about 40 wt %, about 45 wt %, about 50 wt %, about 55 wt %, about 60 wt %, about 65 wt %, about 70 wt %, about 75 wt %, about 80 wt %, about 85 wt %, about 90 wt %, about 95 wt %, about 96 wt %, about 97 wt %, about 98 wt %, or about 99 wt % of the combined dry weight of total lipids in a composition (e.g., a liposomal composition).

In embodiments, a composition (e.g., a liposomal delivery vehicle such as a lipid nanoparticle) comprises about 0.1 wt % to about 20 wt % (e.g., about 0.1 wt % to about 15 wt %) of a compound described herein (e.g., a compound of Formula (A′), (A), (I), (I-a), (I-a′), (I-b), (I-b′), (I-c), (I-c′), (I-c-1), (I-c′-1), (I-c-2), (I-c′-2), (I-d), (I-d′), (I-d-1), (I-d-2), (I-e), (I-e′), (I-e-1), (I-e-2), (I-f), (I-f′), (II), (II-a), (II-a′), (III), (III′), (III-a), (III-a′), (III-b), (III-b′), (III-c), (III-c-1), (III-c′-1), (III-c-2), (III-c′-2), (III-c′), (III-d), (III-d′), (III-d-1), (III-d-2), (III-e), (III-e′), (III-e-1), (III-e-2), (III-f), (III-f′), (IV), (IV-a), or (IV-a′) such as any of Compounds 1-552). In embodiments, a delivery vehicle (e.g., a liposomal delivery vehicle such as a lipid nanoparticle) comprises about 0.5 wt %, about 1 wt %, about 3 wt %, about 5 wt %, or about 10 wt % a compound described herein (e.g., a compound of Formula (A′), (A), (1), (I-a), (I-a′), (I-b), (I-b′), (I-c), (I-c′), (I-c-1), (I-c′-1), (I-c-2), (I-c′-2), (I-d), (I-d′), (I-d-1), (I-d-2), (I-e), (I-e′), (I-e-1), (I-e-2), (I-f), (I-f′), (II), (II-a), (II-a′), (III), (III′), (III-a), (III-a′), (III-b), (III-b′), (III-c), (III-c-1), (III-c′-1), (III-c-2), (III-c′-2), (III-c′), (III-d), (III-d′), (III-d-1), (III-d-2), (III-e), (III-e′), (III-e-1), (III-e-2), (III-f), (III-f), (IV), (IV-a), or (IV-a′) such as any of Compounds 1-552). In embodiments, a delivery vehicle (e.g., a liposomal delivery vehicle such as a lipid nanoparticle) comprises up to about 0.5 wt %, about 1 wt %, about 3 wt %, about 5 wt %, about 10 wt %, about 15 wt %, or about 20 wt % of a compound described herein (e.g., a compound of Formula (A′), (A), (I), (I-a), (I-a′), (I-b), (I-b′), (I-c), (I-c′), (I-c-1), (I-c′-1), (I-c-2), (I-c′-2), (I-d), (I-d′), (I-d-1), (I-d-2), (I-e), (I-e′), (I-e-1), (I-e-2), (I-f), (I-f′) (II), (II-a), (II-a′), (III), (III′), (III-a), (III-a′), (III-b), (III-b′), (III-c), (III-c-1), (III-c′-1), (III-c-2), (III-c′-2), (III-c′), (III-d), (III-d′), (III-d-1), (III-d-2), (III-e), (III-e′), (III-e-1), (III-e-2), (III-f), (III-f′), (IV), (IV-a), or (IV-a′) such as any of Compounds 1-552). In embodiments, the percentage results in an improved beneficial effect (e.g., improved delivery to targeted tissues such as the liver or the lung).

The amount of a compound as described herein (e.g., a compound of Formula (A′), (A), (I), (I-a), (I-a′), (I-b), (I-b′), (I-c), (I-c′), (I-c-1), (I-c′-1), (I-c-2), (I-c′-2), (I-d), (I-d′), (I-d-1), (I-d-2), (I-e), (I-e′), (I-e-1), (I-e-2), (I-f), (I-f′), (II), (II-a), (II-a′), (III), (III′), (III-a), (III-a′), (III-b), (III-b′), (III-c), (III-c-1), (III-c′-1), (III-c-2), (III-c′-2), (III-c′), (III-d), (III-d′), (III-d-1), (III-d-2), (III-e), (III-e′), (III-e-1), (III-e-2), (III-f), (III-f′), (IV), (IV-a), or (IV-a′) such as any of Compounds 1-552) in a composition also can be described as a percentage (“mol %”) of the combined molar amounts of total lipids of a composition (e.g., the combined molar amounts of all lipids present in a liposomal delivery vehicle).

In embodiments of pharmaceutical compositions described herein, a compound as described herein (e.g., a compound of Formula (A′), (A), (I), (I-a), (I-a′), (I-b), (I-b′), (I-c), (I-c′), (I-c-1), (I-c′-1), (I-c-2), (I-c′-2), (I-d), (I-d′), (I-d-1), (I-d-2), (I-e), (I-e′), (I-e-1), (I-e-2), (I-f), (I-f′), (II), (II-a), (II-a′), (III), (III′), (III-a), (III-a′), (III-b), (III-b′), (III-c), (III-c-1), (III-c′-1), (III-c-2), (III-c′-2), (III-c′), (III-d), (III-d′), (III-d-1), (III-d-2), (III-e), (III-e′), (III-e-1), (III-e-2), (III-f), (III-f′), (IV), (IV-a), or (IV-a′) such as any of Compounds 1-552) is present in an amount that is about 0.5 mol % to about 50 mol % (e.g., about 0.5 mol % to about 30 mol %) of the combined molar amounts of all lipids present in a composition such as a liposomal delivery vehicle.

In embodiments, a compound of Formula (A′), (A), (I), (I-a), (I-a′), (I-b), (I-b′), (I-c), (I-c′), (I-c-1), (I-c′-1), (I-c-2), (I-c′-2), (I-d), (I-d′), (I-d-1), (I-d-2), (I-e), (I-e′), (I-e-1), (I-e-2), (I-f), (I-f′), (II), (II-a), (II-a′), (III), (III′), (III-a), (III-a′), (III-b), (III-b′), (III-c), (III-c-1), (III-c′-1), (III-c-2), (III-c′-2), (III-c′), (III-d), (III-d′), (III-d-1), (III-d-2), (III-e), (III-e′), (III-e-1), (III-e-2), (III-f), (III-f′), (IV), (IV-a), or (IV-a′) such as any of Compounds 1-552) is present in an amount that is about 0.5 mol % to about 5 mol %, about 1 mol % to about 10 mol %, about 5 mol % to about 20 mol %, about 10 mol % to about 20 mol %, about 20 mol % to about 30 mol %, about 30 mol % to about 40 mol %, about 40 mol % to about 50 mol %, or about 50 mol % to about 60 mol % of the combined molar amounts of all lipids present in a composition such as a liposomal delivery vehicle. In embodiments, a compound as described herein (e.g., a compound of Formula (A′), (A), (I), (I-a), (I-a′), (I-b), (I-b′), (I-c), (I-c′), (I-c-1), (I-c′-1), (I-c-2), (I-c′-2), (I-d), (I-d′), (I-d-1), (I-d-2), (I-e), (I-e′), (I-e-1), (I-e-2), (I-f), (I-f′), (II), (II-a), (II-a′), (III), (III′), (III-a), (III-a′), (III-b), (III-b′), (III-c), (III-c-1), (III-c′-1), (III-c-2), (III-c′-2), (III-c′), (III-d), (III-d′), (III-d-1), (III-d-2), (III-e), (III-e′), (III-e-1), (III-e-2), (III-f), (III-f′), (IV), (IV-a), or (IV-a′) such as any of Compounds 1-552) is present in an amount that is about about 1 mol % to about 50 mol %, about 1 mol % to about 40 mol %, 1 mol % to about 30 mol %, about 1 mol % to about 20 mol %, about 1 mol % to about 15 mol %, about 1 mol % to about 10 mol %, or about 5 mol % to about 25 mol % of the combined dry weight of all lipids present in a composition such as a liposomal delivery vehicle

In certain embodiments, a compound as described herein (e.g., a compound of Formula (A′), (A), (1), (I-a), (I-a′), (I-b), (I-b′), (I-c), (I-c′), (I-c-1), (I-c′-1), (I-c-2), (I-c′-2), (I-d), (I-d′), (I-d-1), (I-d-2), (I-e), (I-e′), (I-e-1), (I-e-2), (I-f), (I-f′), (II), (II-a), (II-a′), (III), (III′), (III-a), (III-a′), (III-b), (III-b′), (III-c), (III-c-1), (III-c′-1), (III-c-2), (III-c′-2), (III-c′), (III-d), (III-d′), (III-d-1), (III-d-2), (III-e), (III-e′), (III-e-1), (III-e-2), (III-f), (III-f), (IV), (IV-a), or (IV-a′) such as any of Compounds 1-552) can comprise from about 0.1 mol % to about 50 mol %, or from 0.5 mol % to about 50 mol %, or from about 1 mol % to about 25 mol %, or from about 1 mol % to about 10 mol % of the total amount of lipids in a composition (e.g., a liposomal delivery vehicle).

In certain embodiments, a compound as described herein (e.g., a compound of Formula (A′), (A), (1), (I-a), (I-a′), (I-b), (I-b′), (I-c), (I-c′), (I-c-1), (I-c′-1), (I-c-2), (I-c′-2), (I-d), (I-d′), (I-d-1), (I-d-2), (I-e), (I-e′), (I-e-1), (I-e-2), (I-f), (I-f′) (II), (II-a), (II-a′), (III), (III′), (III-a), (III-a′), (III-b), (III-b′), (III-c), (III-c-1), (III-c′-1), (III-c-2), (III-c′-2), (III-c′), (III-d), (III-d′), (III-d-1), (III-d-2), (III-e), (III-e′), (III-e-1), (III-e-2), (III-f), (III-f′), (IV), (IV-a), or (IV-a′) such as any of Compounds 1-552) can comprise greater than about 0.1 mol %, or greater than about 0.5 mol %, or greater than about 1 mol %, or greater than about 5 mol %, or greater than about 10 mol %, or greater than about 15 mol %, or greater than about 20 mol %, or greater than 25 mol %, or greater than 30 mol %, or greater than 35 mol %, or greater than 40 mol %, or greater than 45 mol %, or greater than 50 mol % of the total amount of lipids in the lipid nanoparticle.

In certain embodiments, a compound as described (e.g., a compound of Formula (A′), (A), (I), (I-a), (I-a′), (I-b), (I-b′), (I-c), (I-c′), (I-c-1), (I-c′-1), (I-c-2), (I-c′-2), (I-d), (I-d′), (I-d-1), (I-d-2), (I-e), (I-e′), (I-e-1), (I-e-2), (I-f), (I-f), (II), (II-a), (II-a′), (III), (III′), (III-a), (III-a′), (III-b), (III-b′), (III-c), (III-c-1), (III-c′-1), (III-c-2), (III-c′-2), (III-c′), (III-d), (III-d′), (III-d-1), (III-d-2), (III-e), (III-e′), (III-e-1), (III-e-2), (III-f), (III-f′), (IV), (IV-a), or (IV-a′) such as any of Compounds 1-552) can comprise less than about 50 mol %, or less than about 45 mol %, or less than about 40 mol % or less than about 30%, less than about 25 mol %, or less than about 20 mol %, or less than about 10 mol %, or less than about 5 mol %, or less than about 1 mol % of the total amount of lipids in a composition (e.g., a liposomal delivery vehicle).

In embodiments, the amount of a compound as described herein (e.g., a compound of Formula (A′), (A), (I), (I-a), (I-a′), (I-b), (I-b′), (I-c), (I-c′), (I-c-1), (I-c′-1), (I-c-2), (I-c′-2), (I-d), (I-d′), (I-d-1), (I-d-2), (I-e), (I-e′), (I-e-1), (I-e-2), (I-f), (I-f), (II), (II-a), (II-a′), (III), (III′), (III-a), (III-a′), (III-b), (III-b′), (III-c), (III-c-1), (III-c′-1), (III-c-2), (III-c′-2), (III-c′), (III-d), (III-d′), (III-d-1), (III-d-2), (III-e), (III-e′), (III-e-1), (III-e-2), (III-f), (III-f′), (IV), (IV-a), or (IV-a′) such as any of Compounds 1-552) is present in an amount that is at least about 5 mol %, about 10 mol %, about 15 mol %, about 20 mol %, about 25 mol %, about 30 mol %, about 35 mol %, about 40 mol %, about 45 mol %, about 50 mol %, about 55 mol %, about 60 mol %, about 65 mol %, about 70 mol %, about 75 mol %, about 80 mol %, about 85 mol %, about 90 mol %, about 95 mol %, about 96 mol %, about 97 mol %, about 98 mol %, or about 99 mol % of the combined dry weight of total lipids in a composition (e.g., a liposomal composition).

In embodiments, the amount of a compound as described herein (e.g., a compound of Formula (A′), (A), (1), (I-a), (I-a′), (I-b), (I-b′), (I-c), (I-c′), (I-c-1), (I-c′-1), (I-c-2), (I-c′-2), (I-d), (I-d′), (I-d-1), (I-d-2), (I-e), (I-e′), (I-e-1), (I-e-2), (I-f), (I-f), (II), (II-a), (II-a′), (III), (III′), (III-a), (III-a′), (III-b), (III-b′), (III-c), (III-c-1), (III-c′-1), (III-c-2), (III-c′-2), (III-c′), (III-d), (III-d′), (III-d-1), (III-d-2), (III-e), (III-e′), (III-e-1), (III-e-2), (III-f), (III-f′), (IV), (IV-a), or (IV-a′) such as any of Compounds 1-552) is present in an amount that is no more than about 5 mol %, about 10 mol %, about 15 mol %, about 20 mol %, about 25 mol %, about 30 mol %, about 35 mol %, about 40 mol %, about 45 mol %, about 50 mol %, about 55 mol %, about 60 mol %, about 65 mol %, about 70 mol %, about 75 mol %, about 80 mol %, about 85 mol %, about 90 mol %, about 95 mol %, about 96 mol %, about 97 mol %, about 98 mol %, or about 99 mol % of the combined dry weight of total lipids in a composition (e.g., a liposomal composition).

In embodiments, the percentage results in an improved beneficial effect (e.g., improved delivery to targeted tissues such as the liver or the lung).

In embodiments, a composition further comprises one more lipids (e.g., one more lipids selected from the group consisting of one or more cationic lipids, one or more non-cationic lipids, one or more cholesterol-based lipids, and one or more PEG-modified lipids).

In certain embodiments, such pharmaceutical (e.g., liposomal) compositions comprise one or more of a PEG-modified lipid, a non-cationic lipid and a cholesterol lipid. In embodiments, such pharmaceutical (e.g., liposomal) compositions comprise: one or more PEG-modified lipids; one or more non-cationic lipids; and one or more cholesterol lipids. In embodiments, such pharmaceutical (e.g., liposomal) compositions comprise: one or more PEG-modified lipids and one or more cholesterol lipids.

In embodiments, a composition (e.g., lipid nanoparticle) that encapsulates a nucleic acid (e.g., mRNA encoding a peptide or protein) comprises one or more compounds as described herein (e.g., a compound of Formula (A′), (A), (I), (I-a), (I-a′), (I-b), (I-b′), (I-c), (I-c′), (I-c-1), (I-c′-1), (I-c-2), (I-c′-2), (I-d), (I-d′), (I-d-1), (I-d-2), (I-e), (I-e′), (I-e-1), (I-e-2), (I-f), (I-f′), (II), (II-a), (II-a′), (III), (III′), (III-a), (III-a′), (III-b), (III-b′), (III-c), (III-c-1), (III-c′-1), (III-c-2), (III-c′-2), (III-c′), (III-d), (III-d′), (III-d-1), (III-d-2), (III-e), (III-e′), (III-e-1), (III-e-2), (III-f), (III-f′), (IV), (IV-a), or (IV-a′) such as any of Compounds 1-552) and one or more lipids selected from the group consisting of a cationic lipid, a non-cationic lipid, and a PEGylated lipid.

In embodiments, a composition (e.g., lipid nanoparticle) that encapsulates a nucleic acid (e.g., mRNA encoding a peptide or protein) comprises one or more compound as described herein (e.g., a compound of Formula (A′), (A), (I), (I-a), (I-a′), (I-b), (I-b′), (I-c), (I-c′), (I-c-1), (I-c′-1), (I-c-2), (I-c′-2), (I-d), (I-d′), (I-d-1), (I-d-2), (I-e), (I-e′), (I-e-1), (I-e-2), (I-f), (I-f′), (II), (II-a), (II-a′), (III), (III′), (III-a), (III-a′), (III-b), (III-b′), (III-c), (III-c-1), (III-c′-1), (III-c-2), (III-c′-2), (III-c′), (III-d), (III-d′), (III-d-1), (III-d-2), (III-e), (III-e′), (III-e-1), (III-e-2), (III-f), (III-f′), (IV), (IV-a), or (IV-a′) such as any of Compounds 1-552); one or more lipids selected from the group consisting of a cationic lipid, a non-cationic lipid, and a PEGylated lipid; and further comprises a cholesterol-based lipid.

In embodiments, a lipid nanoparticle that encapsulates a nucleic acid (e.g., mRNA encoding a peptide or protein) comprises one or more compound as described herein ((e.g., a compound of Formula (A′), (A), (1), (I-a), (I-a′), (I-b), (I-b′), (I-c), (I-c′), (I-c-1), (I-c′-1), (I-c-2), (I-c′-2), (I-d), (I-d′), (I-d-1), (I-d-2), (I-e), (I-e′), (I-e-1), (I-e-2), (I-f), (I-f′), (II), (II-a), (II-a′), (III), (III′), (III-a), (III-a′), (III-b), (III-b′), (III-c), (III-c-1), (III-c′-1), (III-c-2), (III-c′-2), (III-c′), (III-d), (III-d′), (III-d-1), (III-d-2), (III-e), (III-e′), (III-e-1), (III-e-2), (III-f), (III-f′), (IV), (IV-a), or (IV-a′) such as any of Compounds 1-552), as well as one or more lipids selected from the group consisting of a cationic lipid, a non-cationic lipid, a PEGylated lipid, and a cholesterol-based lipid.

According to various embodiments, the selection of cationic lipids, non-cationic lipids and/or PEG-modified lipids which comprise the lipid nanoparticle, as well as the relative molar ratio of such lipids to each other, is based upon the characteristics of the selected lipid(s), the nature of the intended target cells, the characteristics of the mRNA to be delivered. Additional considerations include, for example, the saturation of the alkyl chain, as well as the size, charge, pH, pKa, fusogenicity and toxicity of the selected lipid(s). Thus, the molar ratios may be adjusted accordingly.

Cationic Lipids

In addition to any of the compounds as described herein ((e.g., a compound of Formula (A′), (A), (I), (I-a), (I-a′), (I-b), (I-b′), (I-c), (I-c′), (I-c-1), (I-c′-1), (I-c-2), (I-c′-2), (I-d), (I-d′), (I-d-1), (I-d-2), (I-e), (I-e′), (I-e-1), (I-e-2), (I-f), (I-f′), (II), (II-a), (II-a′), (III), (III′), (III-a), (III-a′), (III-b), (III-b′), (III-c), (III-c-1), (III-c′-1), (III-c-2), (III-c′-2), (III-c′), (III-d), (III-d′), (III-d-1), (III-d-2), (III-e), (III-e′), (III-e-1), (III-e-2), (III-f), (III-f′), (IV), (IV-a), or (IV-a′) such as any of Compounds 1-552), a composition may comprise one or more additional cationic lipids.

In some embodiments, liposomes may comprise one or more additional cationic lipids. As used herein, the phrase “cationic lipid” refers to any of a number of lipid species that have a net positive charge at a selected pH, such as physiological pH. Several cationic lipids have been described in the literature, many of which are commercially available.

Suitable cationic lipids for use in the compositions and methods of the invention include the cationic lipids as described in International Patent Publication WO 2010/144740, which is incorporated herein by reference. In certain embodiments, the compositions and methods of the present invention include a cationic lipid, (6Z,9Z,28Z,31Z)-heptatriaconta-6,9,28,31-tetraen-19-yl 4-(dimethylamino) butanoate, having a compound structure of:

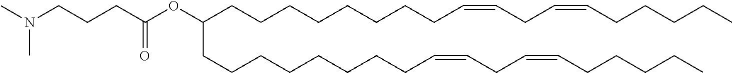 [SMILES: CCCCC/C=C\C/C=C\CCCCCCCCC(CCCCCCCC/C=C\C/C=C\CCCCC)OC(=O)CCCN(C)C]

and pharmaceutically acceptable salts thereof.

Other suitable cationic lipids for use in the compositions and methods of the present invention include ionizable cationic lipids as described in International Patent Publication WO 2013/149140, which is incorporated herein by reference. In some embodiments, the compositions and methods of the present invention include a cationic lipid of one of the following formulas:

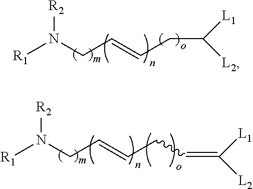

or a pharmaceutically acceptable salt thereof, wherein R₁ and R₂ are each independently selected from the group consisting of hydrogen, an optionally substituted, variably saturated or unsaturated C₁-C₂₀ alkyl and an optionally substituted, variably saturated or unsaturated C₆-C₂₀ acyl; wherein L₁ and L₂ are each independently selected from the group consisting of hydrogen, an optionally substituted C₁-C₃₀ alkyl, an optionally substituted variably unsaturated C₁-C₃₀ alkenyl, and an optionally substituted C₁-C₃₀ alkynyl; wherein m and o are each independently selected from the group consisting of zero and any positive integer (e.g., where m is three); and wherein n is zero or any positive integer (e.g., where n is one). In certain embodiments, the compositions and methods of the present invention include the cationic lipid (15Z, 18Z)—N,N-dimethyl-6-(9Z,12Z)-octadeca-9,12-dien-1-yl) tetracosa-15,18-dien-1-amine (“HGT5000”), having a compound structure of:

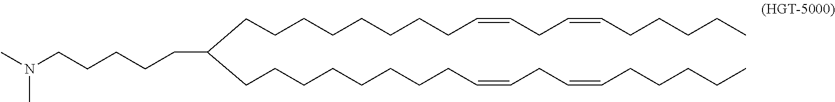 [SMILES: CCCCC/C=C\C/C=C\CCCCCCCCC(CCCCCCCC/C=C\C/C=C\CCCCC)CCCCCN(C)C]

and pharmaceutically acceptable salts thereof. In certain embodiments, the compositions and methods of the present invention include the cationic lipid (15Z, 18Z)—N,N-dimethyl-6-((9Z,12Z)-octadeca-9,12-dien-1-yl) tetracosa-4,15,18-trien-I-amine (“HGT5001”), having a compound structure of:

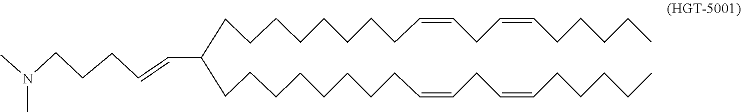 [SMILES: CCCCC/C=C\C/C=C\CCCCCCCCC(/C=C/CCCN(C)C)CCCCCCCC/C=C\C/C=C\CCCCC]

and pharmaceutically acceptable salts thereof. In certain embodiments, the compositions and methods of the present invention include the cationic lipid and (15Z,18Z)—N,N-dimethyl-6-((9Z,12Z)-octadeca-9,12-dien-1-yl) tetracosa-5,15,18-trien-1-amine (“HGT5002”), having a compound structure of:

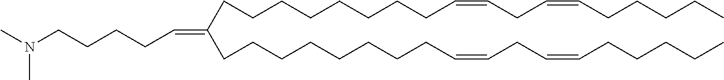 [SMILES: CCCCC/C=C\C/C=C\CCCCCCCCC(=CCCCCN(C)C)CCCCCCCC/C=C\C/C=C\CCCCC]

and pharmaceutically acceptable salts thereof.
Other suitable cationic lipids for use in the compositions and methods of the invention include cationic lipids described as aminoalcohol lipidoids in International Patent Publication WO 2010/053572, which is incorporated herein by reference. In certain embodiments, the compositions and methods of the present invention include a cationic lipid having a compound structure of:

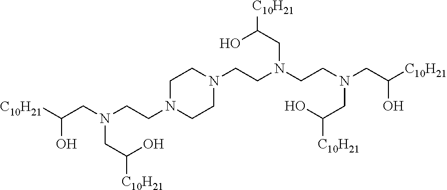

and pharmaceutically acceptable salts thereof.

Other suitable cationic lipids for use in the compositions and methods of the invention include the cationic lipids as described in International Patent Publication WO 2016/118725, which is incorporated herein by reference. In certain embodiments, the compositions and methods of the present invention include a cationic lipid having a compound structure of:

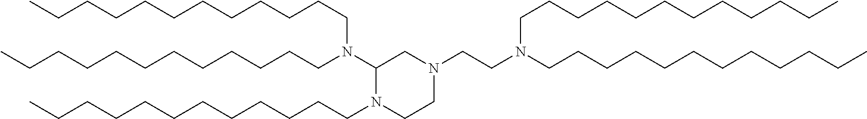 [SMILES: CCCCCCCCCCCCN(CCCCCCCCCCCC)CCN1CCN(CCCCCCCCCCCC)C(N(CCCCCCCCCCCC)CCCCCCCCCCCC)C1]

and pharmaceutically acceptable salts thereof.

Other suitable cationic lipids for use in the compositions and methods of the invention include the cationic lipids as described in International Patent Publication WO 2016/118724, which is incorporated herein by reference. In certain embodiments, the compositions and methods of the present invention include a cationic lipid having a compound structure of:

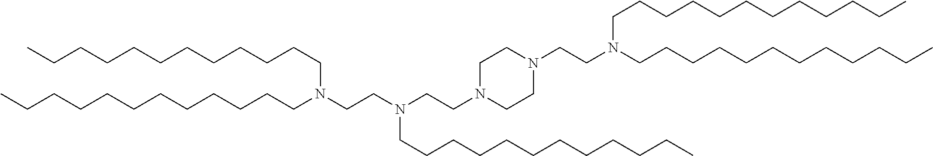 [SMILES: CCCCCCCCCCCCN(CCCCCCCCCCCC)CCN(CCCCCCCCCCCC)CCN1CCN(CCN(CCCCCCCCCCCC)CCCCCCCCCCCC)CC1]

and pharmaceutically acceptable salts thereof.

Other suitable cationic lipids for use in the compositions and methods of the invention include a cationic lipid having the formula of 14,25-ditridecyl 15,18,21,24-tetraaza-octatriacontane, and pharmaceutically acceptable salts thereof.

Other suitable cationic lipids for use in the compositions and methods of the invention include the cationic lipids as described in International Patent Publications WO 2013/063468 and WO 2016/205691, each of which are incorporated herein by reference. In some embodiments, the compositions and methods of the present invention include a cationic lipid of the following formula:

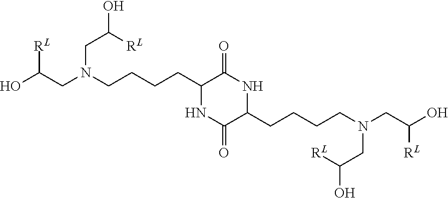

or pharmaceutically acceptable salts thereof, wherein each instance of R^{L} is independently optionally substituted C₆-C₄₀ alkenyl. In certain embodiments, the compositions and methods of the present invention include a cationic lipid having a compound structure of:

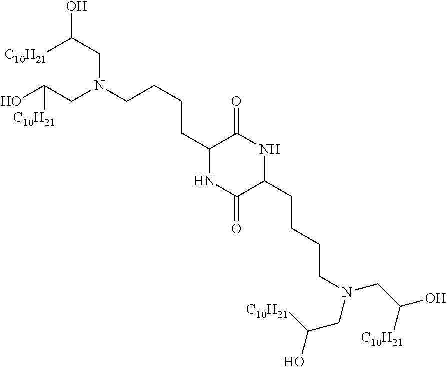

and pharmaceutically acceptable salts thereof. In certain embodiments, the compositions and methods of the present invention include a cationic lipid having a compound structure of:

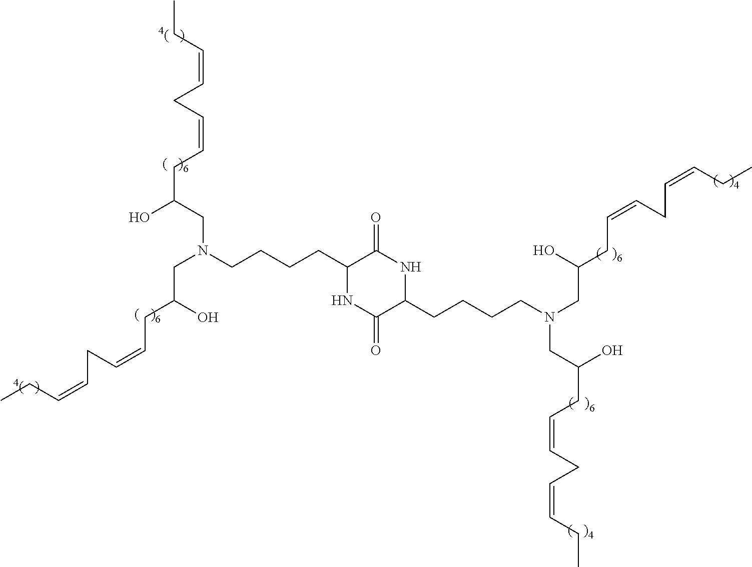

and pharmaceutically acceptable salts thereof. In certain embodiments, the compositions and methods of the present invention include a cationic lipid having a compound structure of:

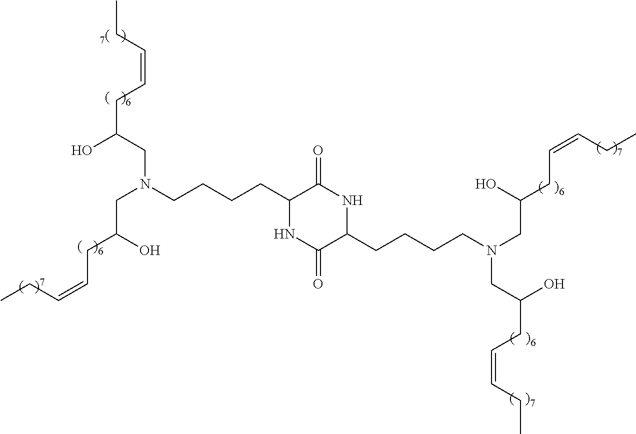

and pharmaceutically acceptable salts thereof. In certain embodiments, the compositions and methods of the present invention include a cationic lipid having a compound structure of:

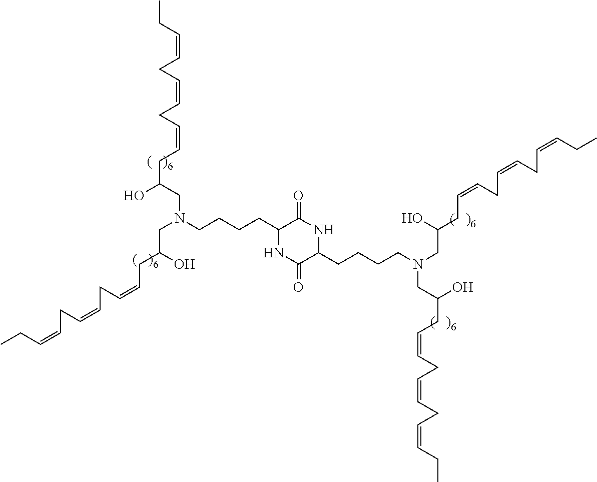

and pharmaceutically acceptable salts thereof.

Other suitable cationic lipids for use in the compositions and methods of the invention include the cationic lipids as described in International Patent Publication WO 2015/184256, which is incorporated herein by reference. In some embodiments, the compositions and methods of the present invention include a cationic lipid of the following formula:

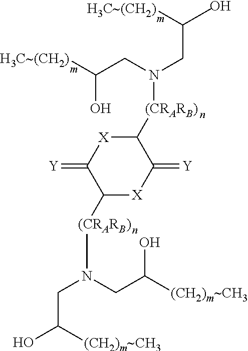

or a pharmaceutically acceptable salt thereof, wherein each X independently is O or S; each Y independently is O or S; each m independently is 0 to 20; each n independently is 1 to 6; each R_{A} is independently hydrogen, optionally substituted C1-50 alkyl, optionally substituted C2-50 alkenyl, optionally substituted C2-50 alkynyl, optionally substituted C3-10 carbocyclyl, optionally substituted 3-14 membered heterocyclyl, optionally substituted C6-14 aryl, optionally substituted 5-14 membered heteroaryl or halogen; and each R_{B} is independently hydrogen, optionally substituted C1-50 alkyl, optionally substituted C2-50 alkenyl, optionally substituted C2-50 alkynyl, optionally substituted C3-10 carbocyclyl, optionally substituted 3-14 membered heterocyclyl, optionally substituted C6-14 aryl, optionally substituted 5-14 membered heteroaryl or halogen. In certain embodiments, the compositions and methods of the present invention include a cationic lipid, “Target 23”, having a compound structure of:

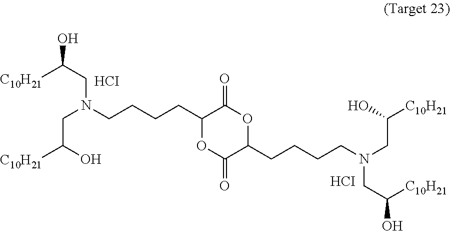

and pharmaceutically acceptable salts thereof.

Other suitable cationic lipids for use in the compositions and methods of the invention include the cationic lipids as described in International Patent Publication WO 2016/004202, which is incorporated herein by reference. In some embodiments, the compositions and methods of the present invention include a cationic lipid having the compound structure:

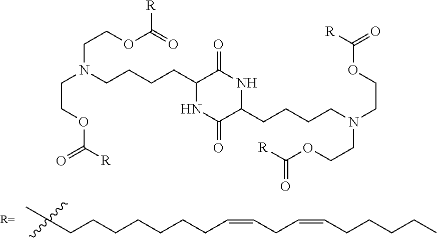

or a pharmaceutically acceptable salt thereof. In some embodiments, the compositions and methods of the present invention include a cationic lipid having the compound structure:

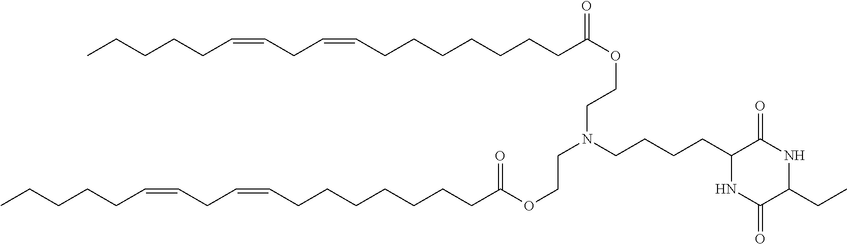 [SMILES: CCCCC/C=C\C/C=C\CCCCCCCC(=O)OCCN(CCCCC1NC(=O)C(CC)NC1=O)CCOC(=O)CCCCCCC/C=C\C/C=C\CCCCC]

or a pharmaceutically acceptable salt thereof. In some embodiments, the compositions and methods of the present invention include a cationic lipid having the compound structure:

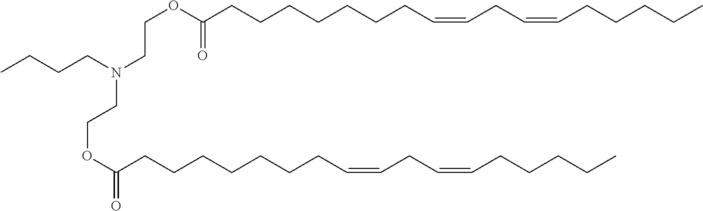 [SMILES: CCCCC/C=C\C/C=C\CCCCCCCC(=O)OCCN(CCCC)CCOC(=O)CCCCCCC/C=C\C/C=C\CCCCC]

or a pharmaceutically acceptable salt thereof.

Other suitable cationic lipids for use in the compositions and methods of the present invention include the cationic lipids as described in J. McClellan, M. C. King, Cell 2010, 141, 210-217 and in Whitehead et al., Nature Communications (2014) 5:4277, which is incorporated herein by reference. In certain embodiments, the cationic lipids of the compositions and methods of the present invention include a cationic lipid having a compound structure of:

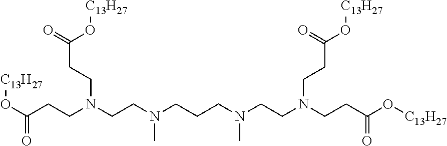

and pharmaceutically acceptable salts thereof.

Other suitable cationic lipids for use in the compositions and methods of the invention include the cationic lipids as described in International Patent Publication WO 2015/199952, which is incorporated herein by reference. In some embodiments, the compositions and methods of the present invention include a cationic lipid having the compound structure:

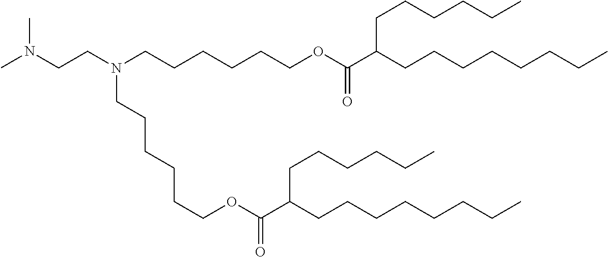 [SMILES: CCCCCCCCC(CCCCCC)C(=O)OCCCCCCN(CCCCCCOC(=O)C(CCCCCC)CCCCCCCC)CCN(C)C]

and pharmaceutically acceptable salts thereof. In some embodiments, the compositions and methods of the present invention include a cationic lipid having the compound structure:

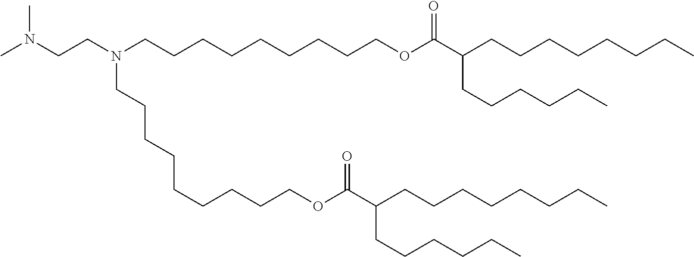 [SMILES: CCCCCCCCC(CCCCCC)C(=O)OCCCCCCCCCN(CCCCCCCCCOC(=O)C(CCCCCC)CCCCCCCC)CCN(C)C]

and pharmaceutically acceptable salts thereof. In some embodiments, the compositions and methods of the present invention include a cationic lipid having the compound structure:

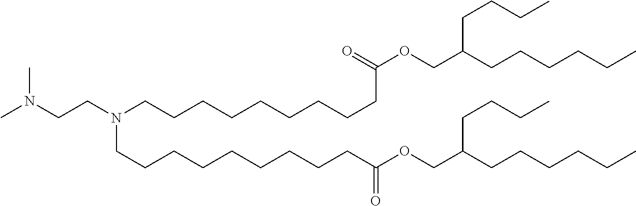 [SMILES: CCCCCCC(CCCC)COC(=O)CCCCCCCCCN(CCCCCCCCCC(=O)OCC(CCCC)CCCCCC)CCN(C)C]

and pharmaceutically acceptable salts thereof. In some embodiments, the compositions and methods of the present invention include a cationic lipid having the compound structure:

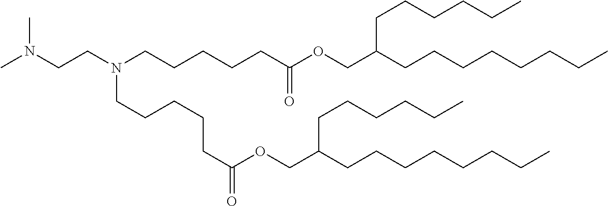 [SMILES: CCCCCCCCC(CCCCCC)COC(=O)CCCCCN(CCCCCC(=O)OCC(CCCCCC)CCCCCCCC)CCN(C)C]

and pharmaceutically acceptable salts thereof. In some embodiments, the compositions and methods of the present invention include a cationic lipid having the compound structure:

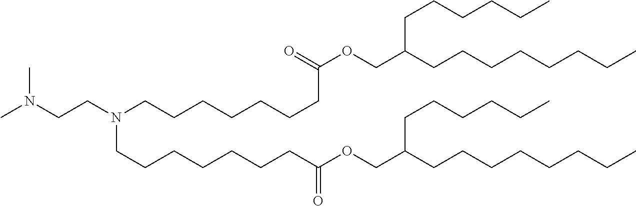 [SMILES: CCCCCCCCC(CCCCCC)COC(=O)CCCCCCCN(CCCCCCCC(=O)OCC(CCCCCC)CCCCCCCC)CCN(C)C]

and pharmaceutically acceptable salts thereof. In some embodiments, the compositions and methods of the present invention include a cationic lipid having the compound structure:

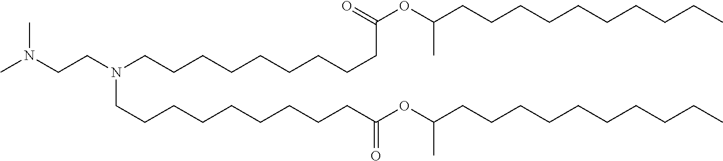 [SMILES: CCCCCCCCCCC(C)OC(=O)CCCCCCCCCN(CCCCCCCCCC(=O)OC(C)CCCCCCCCCC)CCN(C)C]

and pharmaceutically acceptable salts thereof. In some embodiments, the compositions and methods of the present invention include a cationic lipid having the compound structure:

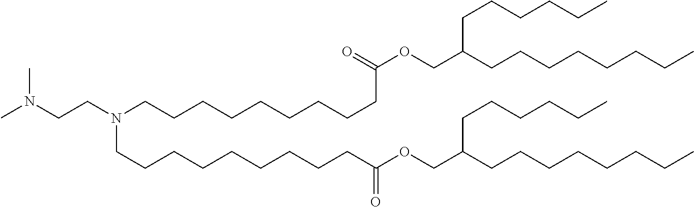 [SMILES: CCCCCCCCC(CCCCCC)COC(=O)CCCCCCCCCN(CCCCCCCCCC(=O)OCC(CCCCCC)CCCCCCCC)CCN(C)C]

and pharmaceutically acceptable salts thereof. In some embodiments, the compositions and methods of the present invention include a cationic lipid having the compound structure:

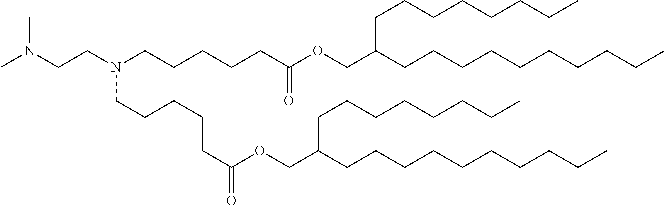

and pharmaceutically acceptable salts thereof. In some embodiments, the compositions and methods of the present invention include a cationic lipid having the compound structure:

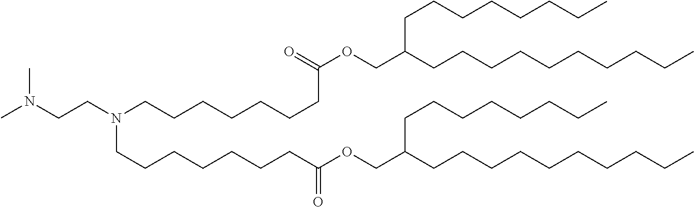 [SMILES: CCCCCCCCCCC(CCCCCCCC)COC(=O)CCCCCCCN(CCCCCCCC(=O)OCC(CCCCCCCC)CCCCCCCCCC)CCN(C)C]

and pharmaceutically acceptable salts thereof. In some embodiments, the compositions and methods of the present invention include a cationic lipid having the compound structure:

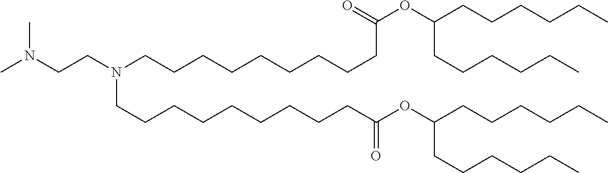 [SMILES: CCCCCCC(CCCCCC)OC(=O)CCCCCCCCCN(CCCCCCCCCC(=O)OC(CCCCCC)CCCCCC)CCN(C)C]

and pharmaceutically acceptable salts thereof. In some embodiments, the compositions and methods of the present invention include a cationic lipid having the compound structure:

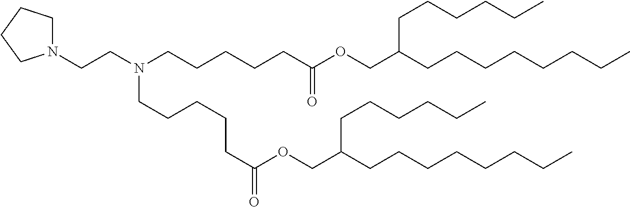 [SMILES: CCCCCCCCC(CCCCCC)COC(=O)CCCCCN(CCCCCC(=O)OCC(CCCCCC)CCCCCCCC)CCN1CCCC1]

and pharmaceutically acceptable salts thereof. In some embodiments, the compositions and methods of the present invention include a cationic lipid having the compound structure:

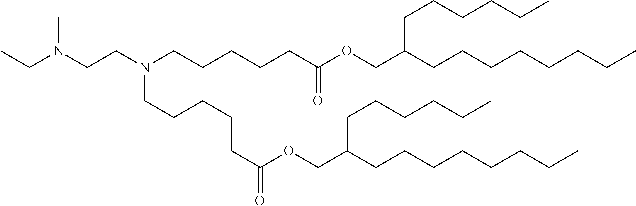 [SMILES: CCCCCCCCC(CCCCCC)COC(=O)CCCCCN(CCCCCC(=O)OCC(CCCCCC)CCCCCCCC)CCN(C)CC]

and pharmaceutically acceptable salts thereof. In some embodiments, the compositions and methods of the present invention include a cationic lipid having the compound structure:

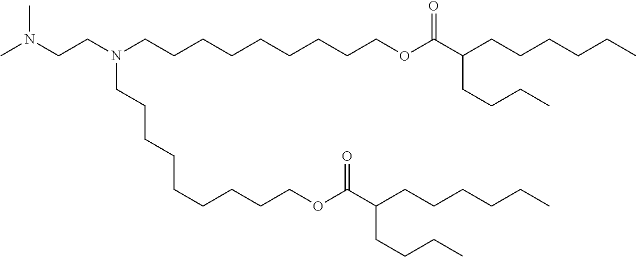 [SMILES: CCCCCCC(CCCC)C(=O)OCCCCCCCCCN(CCCCCCCCCOC(=O)C(CCCC)CCCCCC)CCN(C)C]

and pharmaceutically acceptable salts thereof.

Other suitable cationic lipids for use in the compositions and methods of the invention include the cationic lipids as described in International Patent Publication WO 2017/004143, which is incorporated herein by reference. In some embodiments, the compositions and methods of the present invention include a cationic lipid having the compound structure:

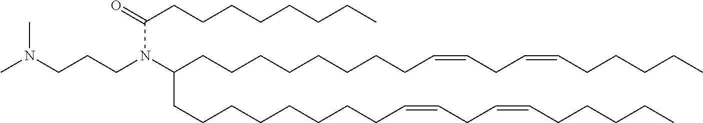

and pharmaceutically acceptable salts thereof. In some embodiments, the compositions and methods of the present invention include a cationic lipid having the compound structure:

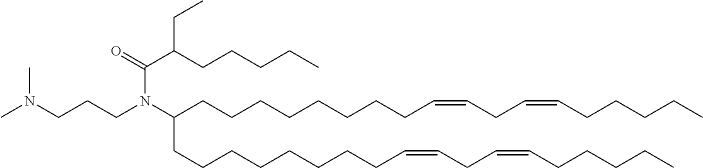 [SMILES: CCCCC/C=C\C/C=C\CCCCCCCCC(CCCCCCCC/C=C\C/C=C\CCCCC)N(CCCN(C)C)C(=O)C(CC)CCCCC]

and pharmaceutically acceptable salts thereof. In some embodiments, the compositions and methods of the present invention include a cationic lipid having the compound structure:

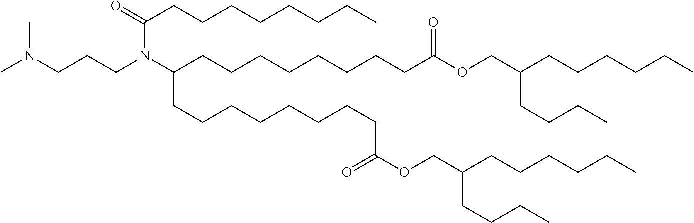 [SMILES: CCCCCCCCC(=O)N(CCCN(C)C)C(CCCCCCCCC(=O)OCC(CCCC)CCCCCC)CCCCCCCCC(=O)OCC(CCCC)CCCCCC]

and pharmaceutically acceptable salts thereof. In some embodiments, the compositions and methods of the present invention include a cationic lipid having the compound structure:

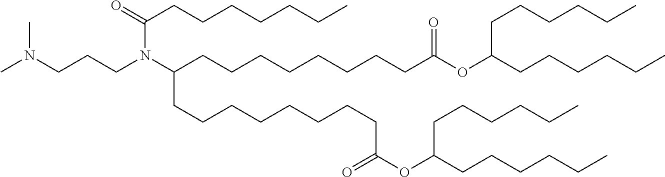 [SMILES: CCCCCCCC(=O)N(CCCN(C)C)C(CCCCCCCCC(=O)OC(CCCCCC)CCCCCC)CCCCCCCCC(=O)OC(CCCCCC)CCCCCC]

and pharmaceutically acceptable salts thereof. In some embodiments, the compositions and methods of the present invention include a cationic lipid having the compound structure:

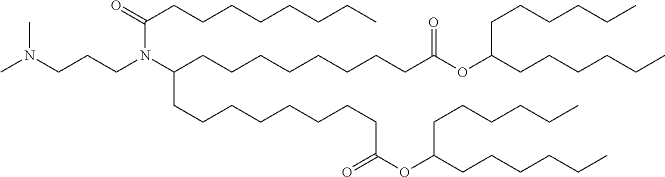 [SMILES: CCCCCCCCC(=O)N(CCCN(C)C)C(CCCCCCCCC(=O)OC(CCCCCC)CCCCCC)CCCCCCCCC(=O)OC(CCCCCC)CCCCCC]

and pharmaceutically acceptable salts thereof. In some embodiments, the compositions and methods of the present invention include a cationic lipid having the compound structure:

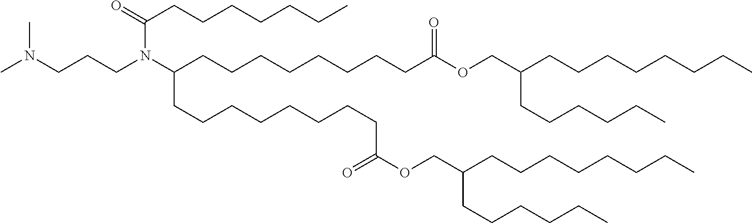 [SMILES: CCCCCCCCC(CCCCCC)COC(=O)CCCCCCCCC(CCCCCCCCC(=O)OCC(CCCCCC)CCCCCCCC)N(CCCN(C)C)C(=O)CCCCCCC]

and pharmaceutically acceptable salts thereof. In some embodiments, the compositions and methods of the present invention include a cationic lipid having the compound structure:

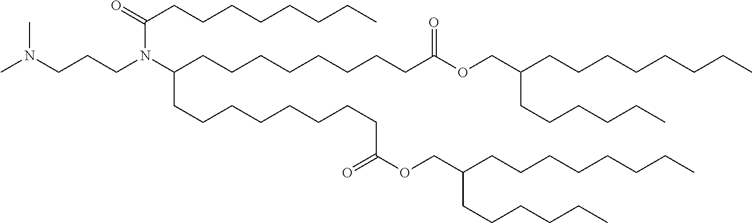 [SMILES: CCCCCCCCC(=O)N(CCCN(C)C)C(CCCCCCCCC(=O)OCC(CCCCCC)CCCCCCCC)CCCCCCCCC(=O)OCC(CCCCCC)CCCCCCCC]

and pharmaceutically acceptable salts thereof. In some embodiments, the compositions and methods of the present invention include a cationic lipid having the compound structure:

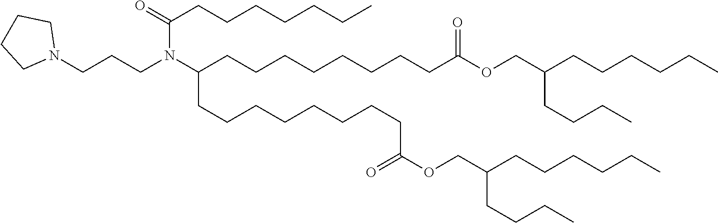 [SMILES: CCCCCCCC(=O)N(CCCN1CCCC1)C(CCCCCCCCC(=O)OCC(CCCC)CCCCCC)CCCCCCCCC(=O)OCC(CCCC)CCCCCC]

and pharmaceutically acceptable salts thereof. In some embodiments, the compositions and methods of the present invention include a cationic lipid having the compound structure:

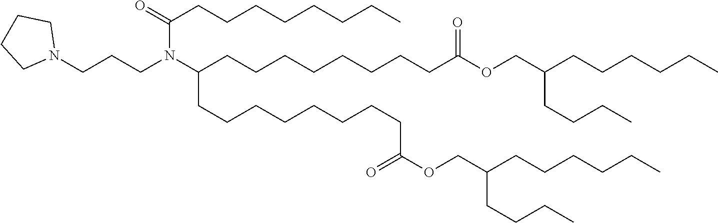 [SMILES: CCCCCCCCC(=O)N(CCCN1CCCC1)C(CCCCCCCCC(=O)OCC(CCCC)CCCCCC)CCCCCCCCC(=O)OCC(CCCC)CCCCCC]

and pharmaceutically acceptable salts thereof. In some embodiments, the compositions and methods of the present invention include a cationic lipid having the compound structure:

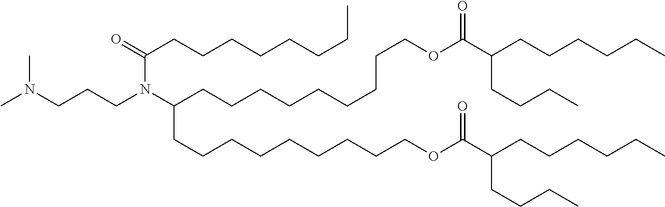 [SMILES: CCCCCCCCC(=O)N(CCCN(C)C)C(CCCCCCCCCOC(=O)C(CCCC)CCCCCC)CCCCCCCCCOC(=O)C(CCCC)CCCCCC]

and pharmaceutically acceptable salts thereof. In some embodiments, the compositions and methods of the present invention include a cationic lipid having the compound structure:

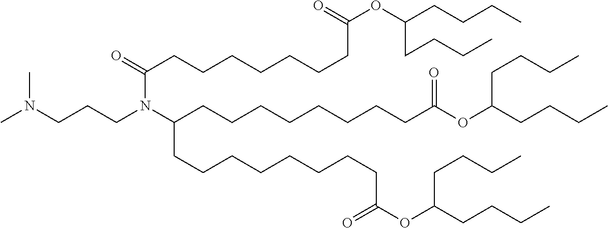 [SMILES: CCCCC(CCCC)OC(=O)CCCCCCCCC(CCCCCCCCC(=O)OC(CCCC)CCCC)N(CCCN(C)C)C(=O)CCCCCCCC(=O)OC(CCCC)CCCC]

and pharmaceutically acceptable salts thereof. In some embodiments, the compositions and methods of the present invention include a cationic lipid having the compound structure:

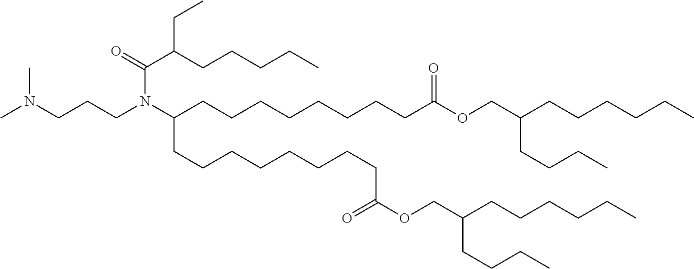 [SMILES: CCCCCCC(CCCC)COC(=O)CCCCCCCCC(CCCCCCCCC(=O)OCC(CCCC)CCCCCC)N(CCCN(C)C)C(=O)C(CC)CCCCC]

and pharmaceutically acceptable salts thereof. In some embodiments, the compositions and methods of the present invention include a cationic lipid having the compound structure:

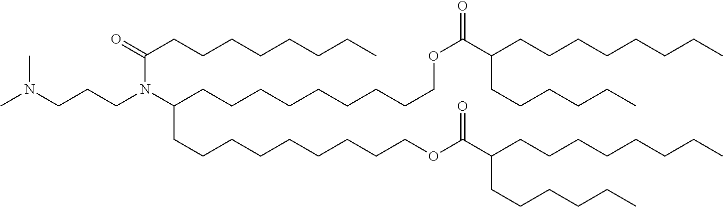 [SMILES: CCCCCCCCC(=O)N(CCCN(C)C)C(CCCCCCCCCOC(=O)C(CCCCCC)CCCCCCCC)CCCCCCCCCOC(=O)C(CCCCCC)CCCCCCCC]

and pharmaceutically acceptable salts thereof. In some embodiments, the compositions and methods of the present invention include a cationic lipid having the compound structure:

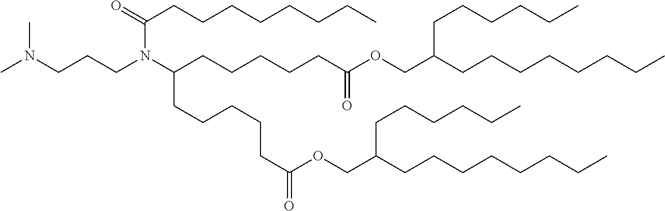 [SMILES: CCCCCCCCC(=O)N(CCCN(C)C)C(CCCCCC(=O)OCC(CCCCCC)CCCCCCCC)CCCCCC(=O)OCC(CCCCCC)CCCCCCCC]

and pharmaceutically acceptable salts thereof. In some embodiments, the compositions and methods of the present invention include a cationic lipid having the compound structure:

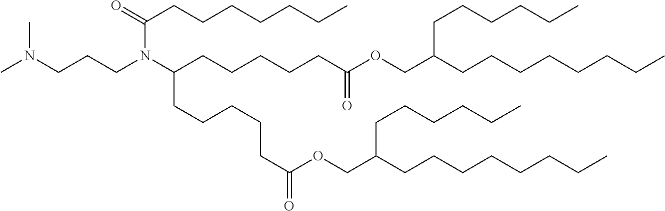 [SMILES: CCCCCCCCC(CCCCCC)COC(=O)CCCCCC(CCCCCC(=O)OCC(CCCCCC)CCCCCCCC)N(CCCN(C)C)C(=O)CCCCCCC]

and pharmaceutically acceptable salts thereof. In some embodiments, the compositions and methods of the present invention include a cationic lipid having the compound structure:

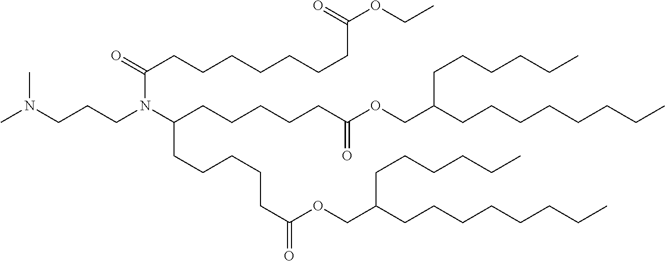 [SMILES: CCCCCCCCC(CCCCCC)COC(=O)CCCCCC(CCCCCC(=O)OCC(CCCCCC)CCCCCCCC)N(CCCN(C)C)C(=O)CCCCCCCC(=O)OCC]

and pharmaceutically acceptable salts thereof. In some embodiments, the compositions and methods of the present invention include a cationic lipid having the compound structure:

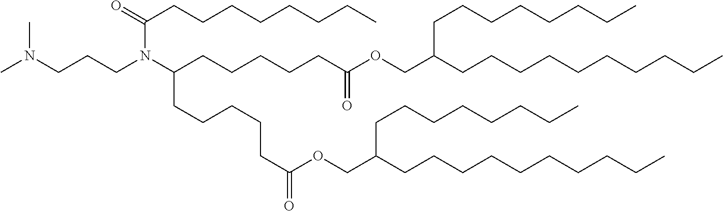 [SMILES: CCCCCCCCCCC(CCCCCCCC)COC(=O)CCCCCC(CCCCCC(=O)OCC(CCCCCCCC)CCCCCCCCCC)N(CCCN(C)C)C(=O)CCCCCCCC]

and pharmaceutically acceptable salts thereof.

Other suitable cationic lipids for use in the compositions and methods of the invention include the cationic lipids as described in International Patent Publication WO 2017/075531, which is incorporated herein by reference. In some embodiments, the compositions and methods of the present invention include a cationic lipid of the following formula:

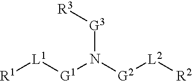

or a pharmaceutically acceptable salt thereof, wherein one of L¹ or L² is —O(C═O)—, —(C═O)O—, —C(═O)—, —O—, —S(O)ₓ, —S—S—, —C(═O)S—, —SC(═O)—, —NR^{a}C(═O)—, —C(═O)NR^{a}—, NR^{a}C(═O)NR^{a}—, —OC(═O)NR^{a}—, or —NR^{a}C(═O)O—; and the other of L¹ or L² is —O(C═O)—, —(C═O)O—, —C(═O)—, —O—, —S(O)ₓ, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O— or a direct bond; G¹ and G² are each independently unsubstituted C₁-C₁₂ alkylene or C₁-C₁₂ alkenylene; G³ is C₁-C₂₄ alkylene, C₁-C₂₄ alkenylene, C₃-C₈ cycloalkylene, C₃-C₈ cycloalkenylene; R^{a} is H or C₁-C₁₂ alkyl; R¹ and R² are each independently C₆-C₂₄ alkyl or C₆-C₂₄ alkenyl; R³ is H, OR⁵, CN, —C(═O)OR⁴, —OC(═O)R⁴ or —NR⁵C(═O)R⁴; R⁴ is C₁-C₁₂ alkyl; R⁵ is H or C₁-C₆ alkyl; and x is 0, 1 or 2.

Other suitable cationic lipids for use in the compositions and methods of the invention include the cationic lipids as described in International Patent Publication WO 2017/117528, which is incorporated herein by reference. In some embodiments, the compositions and methods of the present invention include a cationic lipid having the compound structure:

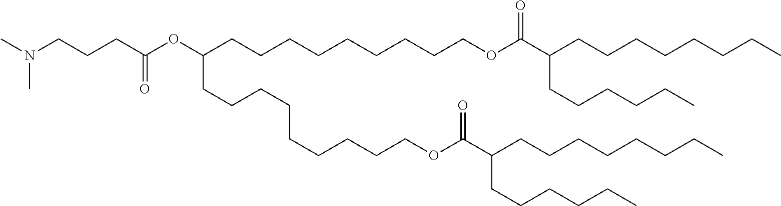 [SMILES: CCCCCCCCC(CCCCCC)C(=O)OCCCCCCCCCC(CCCCCCCCCOC(=O)C(CCCCCC)CCCCCCCC)OC(=O)CCCN(C)C]

and pharmaceutically acceptable salts thereof. In some embodiments, the compositions and methods of the present invention include a cationic lipid having the compound structure:

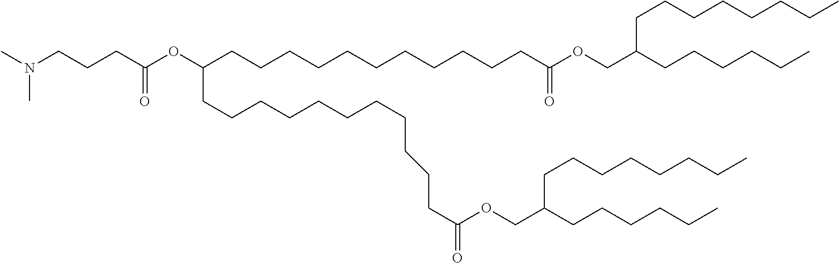 [SMILES: CCCCCCCCC(CCCCCC)COC(=O)CCCCCCCCCCCC(CCCCCCCCCCCC(=O)OCC(CCCCCC)CCCCCCCC)OC(=O)CCCN(C)C]

and pharmaceutically acceptable salts thereof. In some embodiments, the compositions and methods of the present invention include a cationic lipid having the compound structure:

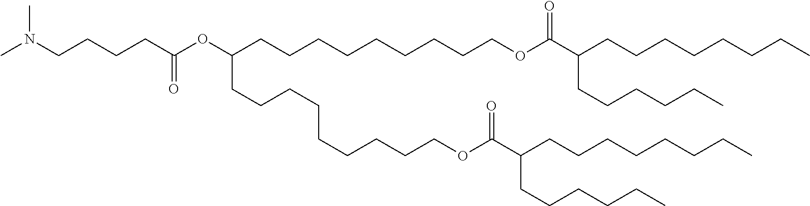 [SMILES: CCCCCCCCC(CCCCCC)C(=O)OCCCCCCCCCC(CCCCCCCCCOC(=O)C(CCCCCC)CCCCCCCC)OC(=O)CCCCN(C)C]

and pharmaceutically acceptable salts thereof.

Other suitable cationic lipids for use in the compositions and methods of the invention include the cationic lipids as described in International Patent Publication WO 2017/049245, which is incorporated herein by reference. In some embodiments, the cationic lipids of the compositions and methods of the present invention include a compound of one of the following formulas:

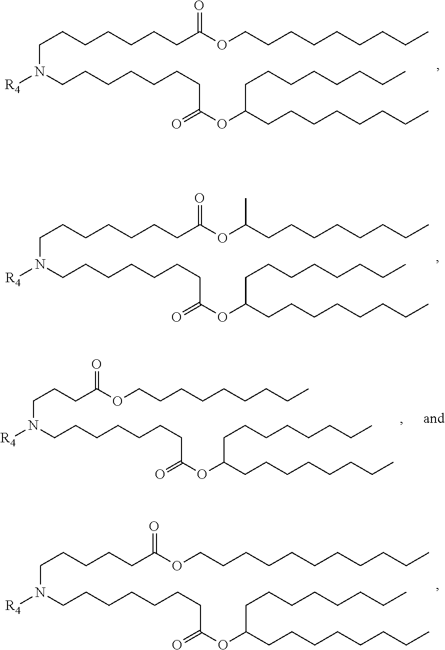

and pharmaceutically acceptable salts thereof. For any one of these four formulas, R₄ is independently selected from —(CH₂)ₙQ and —(CH₂)ₙCHQR; Q is selected from the group consisting of —OR, —OH, —O(CH₂)ₙN(R)₂, —OC(O)R, —CX₃, —CN, —N(R)C(O)R, —N(H)C(O)R, —N(R)S(O)₂R, —N(H)S(O)₂R, —N(R)C(O)N(R)₂, —N(H)C(O)N(R)₂, —N(H)C(O)N(H)(R), —N(R)C(S)N(R)₂, —N(H)C(S)N(R)₂, —N(H)C(S)N(H)(R), and a heterocycle; and n is 1, 2, or 3. In certain embodiments, the compositions and methods of the present invention include a cationic lipid having a compound structure of:

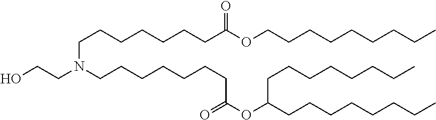 [SMILES: CCCCCCCCCOC(=O)CCCCCCCN(CCO)CCCCCCCC(=O)OC(CCCCCCCC)CCCCCCCC]

and pharmaceutically acceptable salts thereof. In certain embodiments, the compositions and methods of the present invention include a cationic lipid having a compound structure of:

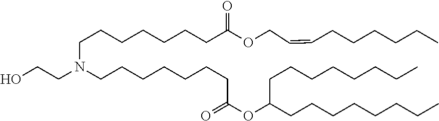 [SMILES: CCCCCC/C=C\COC(=O)CCCCCCCN(CCO)CCCCCCCC(=O)OC(CCCCCCCC)CCCCCCCC]

and pharmaceutically acceptable salts thereof. In certain embodiments, the compositions and methods of the present invention include a cationic lipid having a compound structure of:

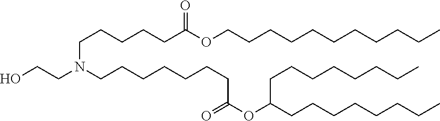 [SMILES: CCCCCCCCCCCOC(=O)CCCCCN(CCO)CCCCCCCC(=O)OC(CCCCCCCC)CCCCCCCC]

and pharmaceutically acceptable salts thereof. In certain embodiments, the compositions and methods of the present invention include a cationic lipid having a compound structure of:

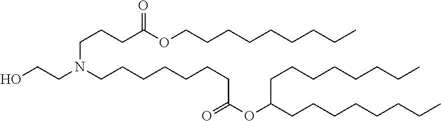 [SMILES: CCCCCCCCCOC(=O)CCCN(CCO)CCCCCCCC(=O)OC(CCCCCCCC)CCCCCCCC]

and pharmaceutically acceptable salts thereof.

Other suitable cationic lipids for use in the compositions and methods of the invention include the cationic lipids as described in International Patent Publication WO 2017/173054 and WO 2015/095340, each of which is incorporated herein by reference. In certain embodiments, the compositions and methods of the present invention include a cationic lipid having a compound structure of:

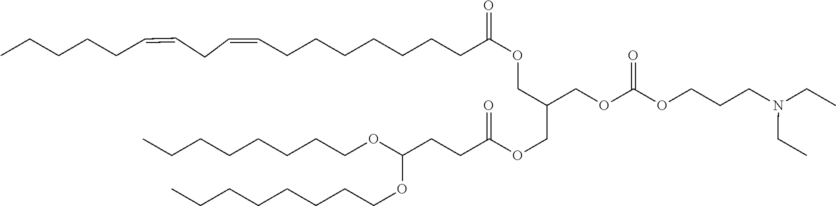 [SMILES: CCCCC/C=C\C/C=C\CCCCCCCC(=O)OCC(COC(=O)CCC(OCCCCCCCC)OCCCCCCCC)COC(=O)OCCCN(CC)CC]

and pharmaceutically acceptable salts thereof. In certain embodiments, the compositions and methods of the present invention include a cationic lipid having a compound structure of:

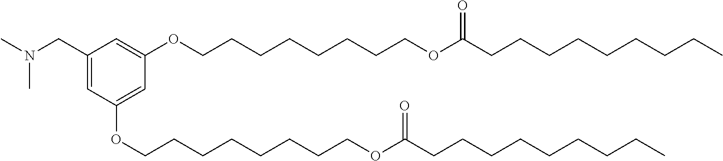 [SMILES: CCCCCCCCCC(=O)OCCCCCCCCOc1cc(CN(C)C)cc(OCCCCCCCCOC(=O)CCCCCCCCC)c1]

and pharmaceutically acceptable salts thereof. In certain embodiments, the compositions and methods of the present invention include a cationic lipid having a compound structure of:

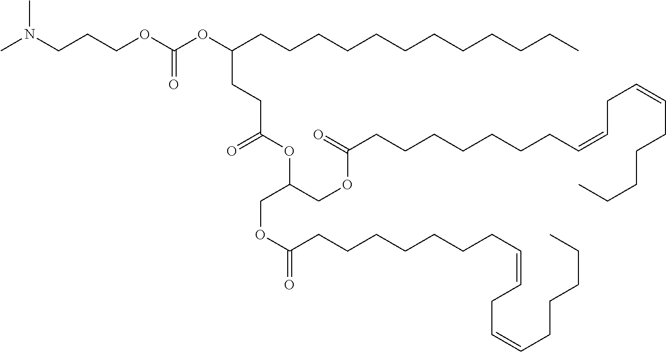 [SMILES: CCCCC/C=C\C/C=C\CCCCCCCC(=O)OCC(COC(=O)CCCCCCC/C=C\C/C=C\CCCCC)OC(=O)CCC(CCCCCCCCCCCC)OC(=O)OCCCN(C)C]

and pharmaceutically acceptable salts thereof. In certain embodiments, the compositions and methods of the present invention include a cationic lipid having a compound structure of:

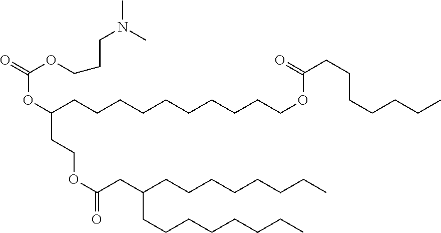 [SMILES: CCCCCCCCC(CCCCCCCC)CC(=O)OCCC(CCCCCCCCCCOC(=O)CCCCCCC)OC(=O)OCCCN(C)C]

and pharmaceutically acceptable salts thereof.

Other suitable cationic lipids for use in the compositions and methods of the present invention include cleavable cationic lipids as described in International Patent Publication WO 2012/170889, which is incorporated herein by reference. In some embodiments, the compositions and methods of the present invention include a cationic lipid of the following formula:

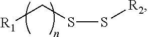

wherein R₁ is selected from the group consisting of imidazole, guanidinium, amino, imine, enamine, an optionally-substituted alkyl amino (e.g., an alkyl amino such as dimethylamino) and pyridyl; wherein R₂ is selected from the group consisting of one of the following two formulas:

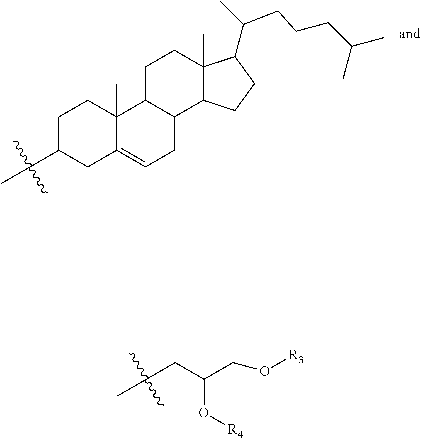

and wherein R₃ and R₄ are each independently selected from the group consisting of an optionally substituted, variably saturated or unsaturated C₆-C₂₀ alkyl and an optionally substituted, variably saturated or unsaturated C₆-C₂₀ acyl; and wherein n is zero or any positive integer (e.g., one, two, three, four, five, six, seven, eight, nine, ten, eleven, twelve, thirteen, fourteen, fifteen, sixteen, seventeen, eighteen, nineteen, twenty or more). In certain embodiments, the compositions and methods of the present invention include a cationic lipid, “HGT4001”, having a compound structure of:

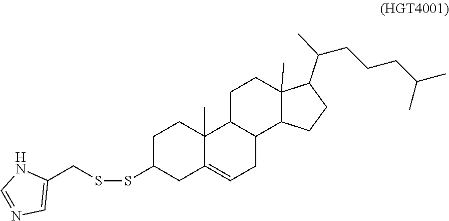 [SMILES: CC(C)CCCC(C)C1CCC2C3CC=C4CC(SSCc5cnc[nH]5)CCC4(C)C3CCC12C]

and pharmaceutically acceptable salts thereof. In certain embodiments, the compositions and methods of the present invention include a cationic lipid, “HGT4002,” having a compound structure of:

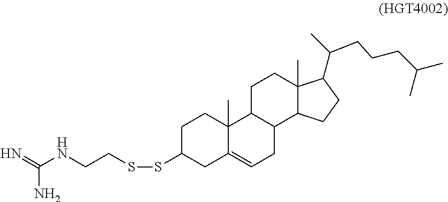 [SMILES: CC(C)CCCC(C)C1CCC2C3CC=C4CC(SSCCNC(=N)N)CCC4(C)C3CCC12C]

and pharmaceutically acceptable salts thereof. In certain embodiments, the compositions and methods of the present invention include a cationic lipid, “HGT4003,” having a compound structure of:

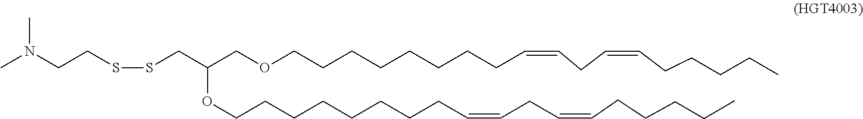 [SMILES: CCCCC/C=C\C/C=C\CCCCCCCCOCC(CSSCCN(C)C)OCCCCCCCC/C=C\C/C=C\CCCCC]

and pharmaceutically acceptable salts thereof. In certain embodiments, the compositions and methods of the present invention include a cationic lipid, “HGT4004,” having a compound structure of:

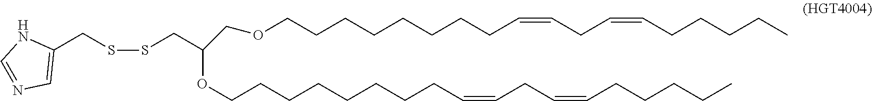 [SMILES: CCCCC/C=C\C/C=C\CCCCCCCCOCC(CSSCc1cnc[nH]1)OCCCCCCCC/C=C\C/C=C\CCCCC]

and pharmaceutically acceptable salts thereof. In certain embodiments, the compositions and methods of the present invention include a cationic lipid “HGT4005,” having a compound structure of:

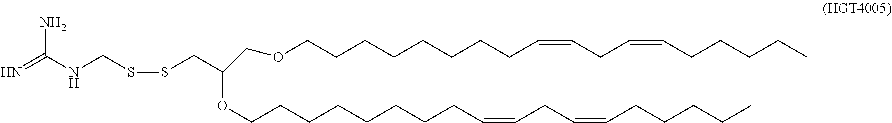 [SMILES: CCCCC/C=C\C/C=C\CCCCCCCCOCC(CSSCNC(=N)N)OCCCCCCCC/C=C\C/C=C\CCCCC]

and pharmaceutically acceptable salts thereof.

In some embodiments, the compositions and methods of the present invention include the cationic lipid, N-[1-(2,3-dioleyloxy)propyl]-N,N,N-trimethylammonium chloride (“DOTMA”). (Feigner et al. (Proc. Nat'l Acad. Sci. 84, 7413 (1987); U.S. Pat. No. 4,897,355, which is incorporated herein by reference). Other cationic lipids suitable for the compositions and methods of the present invention include, for example, 5-carboxyspermylglycinedioctadecylamide (“DOGS”); 2,3-dioleyloxy-N-[2(spermine-carboxamido)ethyl]-N,N-dimethyl-l-propanaminium (“DOSPA”) (Behr et al. Proc. Nat.'l Acad. Sci. 86, 6982 (1989), U.S. Pat. Nos. 5,171,678; 5,334,761); 1,2-Dioleoyl-3-Dimethylammonium-Propane (“DODAP”); 1,2-Dioleoyl-3-Trimethylammonium-Propane (“DOTAP”).

Additional exemplary cationic lipids suitable for the compositions and methods of the present invention also include: 1,2-distearyloxy-N,N-dimethyl-3-aminopropane (“DSDMA”); 1,2-dioleyloxy-N,N-dimethyl-3-aminopropane (“DODMA”); 1,2-dilinoleyloxy-N,N-dimethyl-3-aminopropane (“DLinDMA”); 1,2-dilinolenyloxy-N,N-dimethyl-3-aminopropane (“DLenDMA”); N-dioleyl-N,N-dimethylammonium chloride (“DODAC”); N,N-distearyl-N,N-dimethylammonium bromide (“DDAB”); N-(1,2-dimyristyloxyprop-3-yl)-N,N-dimethyl-N-hydroxyethyl ammonium bromide (“DMRIE”); 3-dimethylamino-2-(cholest-5-en-3-beta-oxybutan-4-oxy)-1-(cis,cis-9,12-octadecadienoxy)propane (“CLinDMA”); 2-[5′-(cholest-5-en-3-beta-oxy)-3′-oxapentoxy)-3-dimethyl-I-(cis,cis-9′,I-2′-octadecadienoxy)propane (“CpLinDMA”); N,N-dimethyl-3,4-dioleyloxybenzylamine (“DMOBA”); 1,2-N,N′-dioleylcarbamyl-3-dimethylaminopropane (“DOcarbDAP”); 2,3-Dilinoleoyloxy-N,N-dimethylpropylamine (“DLinDAP”); 1,2-N,N′-Dilinoleylcarbamyl-3-dimethylaminopropane (“DLincarbDAP”); I,2-Dilinoleoylcarbamyl-3-dimethylaminopropane (“DLinCDAP”); 2,2-dilinoleyl-4-dimethylaminomethyl-[1,3]-dioxolane (“DLin-K-DMA”); 2-((8-[(3β)-cholest-5-en-3-yloxy]octyl)oxy)-N, N-dimethyl-3-[(9Z, 12Z)-octadeca-9, 12-dien-1-yloxy]propane-1-amine (“Octyl-CLinDMA”); (2R)-2-((8-[(3beta)-cholest-5-en-3-yloxy]octyl)oxy)-N, N-dimethyl-3-[(9Z, 12Z)-octadeca-9, 12-dien-1-yloxy]propan-1-amine (“Octyl-CLinDMA (2R)”); (2S)-2-((8-[(3β)-cholest-5-en-3-yloxy]octyl)oxy)-N, N-dimethyl-[(9Z, 12Z)-octadeca-9, 12-dien-1-yloxy]propan-1-amine (“Octyl-CLinDMA (2S)”); 2,2-dilinoleyl-4-dimethylaminoethyl-[1,3]-dioxolane (“DLin-K-XTC2-DMA”); and 2-(2,2-di((9Z,12Z)-octadeca-9,12-dien-1-yl)-1,3-dioxolan-4-yl)-N,N-dimethylethanamine (“DLin-KC2-DMA”) (see, WO 2010/042877, which is incorporated herein by reference; Semple et al., Nature Biotech. 28: 172-176 (2010)). (Heyes, J., et al., J Controlled Release 107: 276-287 (2005); Morrissey, D V., et al; Nat. Biotechnol. 23(8): 1003-1007 (2005); International Patent Publication WO 2005/121348). In some embodiments, one or more of the cationic lipids comprise at least one of an imidazole, dialkylamino, or guanidinium moiety.

In some embodiments, one or more cationic lipids suitable for the compositions and methods of the present invention include 2,2-Dilinoleyl-4-dimethylaminoethyl-[1,3]-dioxolane (“XTC”); (3aR,5s,6aS)—N,N-dimethyl-2,2-di((9Z,12Z)-octadeca-9,12-dienyl)tetrahydro-3aH-cyclopenta[d] [1,3]dioxol-5-amine (“ALNY-100”) and/or 4,7,13-tris(3-oxo-3-(undecylamino)propyl)-N1,N16-diundecyl-4,7,10,13-tetraazahexadecane-1,16-diamide (“NC98-5”).

In some embodiments, the compositions of the present invention include one or more cationic lipids that constitute at least about 5%, 10%, 20%, 30%, 35%, 40%, 45%, 50%, 55%, 60%, 65%, or 70%, measured by weight, of the total lipid content in the composition, e.g., a lipid nanoparticle. In some embodiments, the compositions of the present invention include one or more cationic lipids that constitute at least about 5%, 10%, 20%, 30%, 35%, 40%, 45%, 50%, 55%, 60%, 65%, or 70%, measured as a mol %, of the total lipid content in the composition, e.g., a lipid nanoparticle. In some embodiments, the compositions of the present invention include one or more cationic lipids that constitute about 30-70% (e.g., about 30-65%, about 30-60%, about 30-55%, about 30-50%, about 30-45%, about 30-40%, about 35-50%, about 35-45%, or about 35-40%), measured by weight, of the total lipid content in the composition, e.g., a lipid nanoparticle. In some embodiments, the compositions of the present invention include one or more cationic lipids that constitute about 30-70% (e.g., about 30-65%, about 30-60%, about 30-55%, about 30-50%, about 30-45%, about 30-40%, about 35-50%, about 35-45%, or about 35-40%), measured as mol %, of the total lipid content in the composition, e.g., a lipid nanoparticle.

Non-Cationic/Helper Lipids

In some embodiments, the liposomes contain one or more non-cationic (“helper”) lipids. As used herein, the phrase “non-cationic lipid” refers to any neutral, zwitterionic or anionic lipid. As used herein, the phrase “anionic lipid” refers to any of a number of lipid species that carry a net negative charge at a selected pH, such as physiological pH. Non-cationic lipids include, but are not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 1,2-dierucoyl-sn-glycero-3-phosphoethanolamine (DEPE), phosphatidylserine, sphingolipids, cerebrosides, gangliosides, 16-O-monomethyl PE, 16-O-dimethyl PE, 18-1-trans PE, 1-stearoyl-2-oleoyl-phosphatidyethanolamine (SOPE), or a mixture thereof. In some embodiments, liposomes suitable for use with the invention include DOPE as the non-cationic lipid component. In other embodiments, liposomes suitable for use with the invention include DEPE as the non-cationic lipid component.

In some embodiments, a non-cationic lipid is a neutral lipid, i.e., a lipid that does not carry a net charge in the conditions under which the composition is formulated and/or administered.

In some embodiments, such non-cationic lipids may be used alone, but are preferably used in combination with other lipids, for example, cationic lipids.

In some embodiments, a non-cationic lipid may be present in a molar ratio (mol %) of about 5% to about 90%, about 5% to about 70%, about 5% to about 50%, about 5% to about 40%, about 5% to about 30%, about 10% to about 70%, about 10% to about 50%, or about 10% to about 40% of the total lipids present in a composition. In some embodiments, total non-cationic lipids may be present in a molar ratio (mol %) of about 5% to about 90%, about 5% to about 70%, about 5% to about 50%, about 5% to about 40%, about 5% to about 30%, about 10% to about 70%, about 10% to about 50%, or about 10% to about 40% of the total lipids present in a composition. In some embodiments, the percentage of non-cationic lipid in a liposome may be greater than about 5 mol %, greater than about 10 mol %, greater than about 20 mol %, greater than about 30 mol %, or greater than about 40 mol %. In some embodiments, the percentage total non-cationic lipids in a liposome may be greater than about 5 mol %, greater than about 10 mol %, greater than about 20 mol %, greater than about 30 mol %, or greater than about 40 mol %. In some embodiments, the percentage of non-cationic lipid in a liposome is no more than about 5 mol %, no more than about 10 mol %, no more than about 20 mol %, no more than about 30 mol %, or no more than about 40 mol %. In some embodiments, the percentage total non-cationic lipids in a liposome may be no more than about 5 mol %, no more than about 10 mol %, no more than about 20 mol %, no more than about 30 mol %, or no more than about 40 mol %.

In some embodiments, a non-cationic lipid may be present in a weight ratio (wt %) of about 5% to about 90%, about 5% to about 70%, about 5% to about 50%, about 5% to about 40%, about 5% to about 30%, about 10% to about 70%, about 10% to about 50%, or about 10% to about 40% of the total lipids present in a composition. In some embodiments, total non-cationic lipids may be present in a weight ratio (wt %) of about 5% to about 90%, about 5% to about 70%, about 5% to about 50%, about 5% to about 40%, about 5% to about 30%, about 10% to about 70%, about 10% to about 50%, or about 10% to about 40% of the total lipids present in a composition. In some embodiments, the percentage of non-cationic lipid in a liposome may be greater than about 5 wt %, greater than about 10 wt %, greater than about 20 wt %, greater than about 30 wt %, or greater than about 40 wt %. In some embodiments, the percentage total non-cationic lipids in a liposome may be greater than about 5 wt %, greater than about 10 wt %, greater than about 20 wt %, greater than about 30 wt %, or greater than about 40 wt %. In some embodiments, the percentage of non-cationic lipid in a liposome is no more than about 5 wt %, no more than about 10 wt %, no more than about 20 wt %, no more than about 30 wt %, or no more than about 40 wt %. In some embodiments, the percentage total non-cationic lipids in a liposome may be no more than about 5 wt %, no more than about 10 wt %, no more than about 20 wt %, no more than about 30 wt %, or no more than about 40 wt %.

Cholesterol-Based Lipids

In some embodiments, the liposomes comprise one or more cholesterol-based lipids. For example, suitable cholesterol-based cationic lipids include, for example, DC-Choi (N,N-dimethyl-N-ethylcarboxamidocholesterol), I,4-bis(3-N-oleylamino-propyl)piperazine (Gao, et al. Biochem. Biophys. Res. Comm. 179, 280 (1991); Wolf et al. BioTechniques 23, 139 (1997); U.S. Pat. No. 5,744,335), or imidazole cholesterol ester (ICE), which has the following structure,

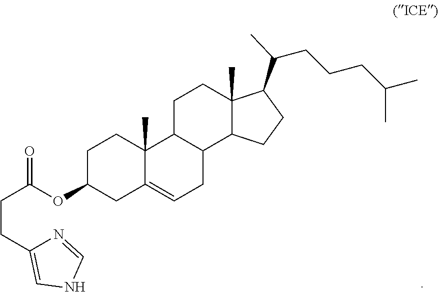 [SMILES: CC(C)CCCC(C)[C@H]1CCC2C3CC=C4C[C@@H](OC(=O)CCc5c[nH]cn5)CC[C@]4(C)C3CC[C@@]21C]

In embodiments, a cholesterol-based lipid is cholesterol.

In some embodiments, the cholesterol-based lipid may comprise a molar ratio (mol %) of about 1% to about 30%, or about 5% to about 20% of the total lipids present in a liposome. In some embodiments, the percentage of cholesterol-based lipid in the lipid nanoparticle may be greater than about 5 mol %, greater than about 10 mol %, greater than about 20 mol %, greater than about 30 mol %, or greater than about 40 mol %. In some embodiments, the percentage of cholesterol-based lipid in the lipid nanoparticle may be no more than about 5 mol %, no more than about 10 mol %, no more than about 20 mol %, no more than about 30 mol %, or no more than about 40 mol %.

In some embodiments, a cholesterol-based lipid may be present in a weight ratio (wt %) of about 1% to about 30%, or about 5% to about 20% of the total lipids present in a liposome. In some embodiments, the percentage of cholesterol-based lipid in the lipid nanoparticle may be greater than about 5 wt %, greater than about 10 wt %, greater than about 20 wt %, greater than about 30 wt %, or greater than about 40 wt %. In some embodiments, the percentage of cholesterol-based lipid in the lipid nanoparticle may be no more than about 5 wt %, no more than about 10 wt %, no more than about 20 wt %, no more than about 30 wt %, or no more than about 40 wt %.

PEGylated Lipids

In some embodiments, the liposome comprises one or more PEGylated lipids.

For example, the use of polyethylene glycol (PEG)-modified phospholipids and derivatized lipids such as derivatized ceramides (PEG-CER), including N-Octanoyl-Sphingosine-1-[Succinyl(Methoxy Polyethylene Glycol)-2000] (C₈ PEG-2000 ceramide) is also contemplated by the present invention, either alone or preferably in combination with other lipid formulations together which comprise the transfer vehicle (e.g., a lipid nanoparticle).

Contemplated PEG-modified lipids include, but are not limited to, a polyethylene glycol chain of up to 5 kDa in length covalently attached to a lipid with alkyl chain(s) of C₆-C₂₀ length. In some embodiments, a PEG-modified or PEGylated lipid is PEGylated cholesterol or PEG-2K. The addition of such components may prevent complex aggregation and may also provide a means for increasing circulation lifetime and increasing the delivery of the lipid-nucleic acid composition to the target tissues, (Klibanov et al. (1990) FEBS Letters, 268 (1): 235-237), or they may be selected to rapidly exchange out of the formulation in vivo (see U.S. Pat. No. 5,885,613). Particularly useful exchangeable lipids are PEG-ceramides having shorter acyl chains (e.g., C₁₄ or C₁₈). Liposomes suitable for use with the invention typically include a PEG-modified lipid such as 1,2-dimyristoyl-rac-glycero-3-methoxypolyethylene glycol-2000 (DMG-PEG2K).

The PEG-modified phospholipid and derivatized lipids of the present invention may comprise a molar ratio from about 0% to about 20%, about 0.5% to about 20%, about 1% to about 15%, about 4% to about 10%, or about 2% of the total lipid present in the liposomal transfer vehicle. In some embodiments, one or more PEG-modified lipids constitute about 4% of the total lipids by molar ratio. In some embodiments, one or more PEG-modified lipids constitute about 5% of the total lipids by molar ratio. In some embodiments, one or more PEG-modified lipids constitute about 6% of the total lipids by molar ratio. In a typical embodiment of the invention, the PEG-modified lipid (e.g., DMG-PEG2K) is present at a molar ratio of about 2% to about 6% of the total lipid present in the liposomal transfer vehicle. In specific embodiments, the PEG-modified lipid (e.g., DMG-PEG2K) is present at a molar ratio of about 3% to about 5% of the total lipid present in the liposomal transfer vehicle. For certain applications, such as pulmonary delivery, liposomes in which the PEG-modified lipid component constitutes about 5% of the total lipids by molar ratio have been found to be particularly suitable. For other applications, such as intravenous delivery, liposomes in which the PEG-modified lipid component constitutes less than about 5% of the total lipids by molar ratio, e.g., 3% of the total lipids by molar ratio, may be particularly suitable.

Amphiphilic Block Copolymers

In some embodiments, a suitable delivery vehicle contains amphiphilic block copolymers (e.g., poloxamers).

Various amphiphilic block copolymers may be used to practice the present invention. In some embodiments, an amphiphilic block copolymer is also referred to as a surfactant or a non-ionic surfactant.

In some embodiments, an amphiphilic polymer suitable for the invention is selected from poloxamers (Pluronic®), poloxamines (Tetronic®), polyoxyethylene glycol sorbitan alkyl esters (polysorbates) and polyvinyl pyrrolidones (PVPs).

Poloxamers

In some embodiments, a suitable amphiphilic polymer is a poloxamer. For example, a suitable poloxamer is of the following structure:

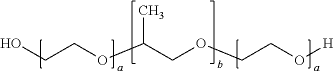

wherein a is an integer between 10 and 150 and b is an integer between 20 and 60. For example, a is about 12 and b is about 20, or a is about 80 and b is about 27, or a is about 64 and b is about 37, or a is about 141 and b is about 44, or a is about 101 and b is about 56.

In some embodiments, a poloxamer suitable for the invention has ethylene oxide units from about 10 to about 150. In some embodiments, a poloxamer has ethylene oxide units from about 10 to about 100.

In some embodiments, a suitable poloxamer is poloxamer 84. In some embodiments, a suitable poloxamer is poloxamer 101. In some embodiments, a suitable poloxamer is poloxamer 105. In some embodiments, a suitable poloxamer is poloxamer 108. In some embodiments, a suitable poloxamer is poloxamer 122. In some embodiments, t a suitable poloxamer is poloxamer 123. In some embodiments, a suitable poloxamer is poloxamer 124. In some embodiments, a suitable poloxamer is poloxamer 181. In some embodiments, a suitable poloxamer is poloxamer 182. In some embodiments, a suitable poloxamer is poloxamer 183. In some embodiments, a suitable poloxamer is poloxamer 184. In some embodiments, a suitable poloxamer is poloxamer 185. In some embodiments, a suitable poloxamer is poloxamer 188. In some embodiments, a suitable poloxamer is poloxamer 212. In some embodiments, a suitable poloxamer is poloxamer 215. In some embodiments, a suitable poloxamer is poloxamer 217. In some embodiments, a suitable poloxamer is poloxamer 231. In some embodiments, a suitable poloxamer is poloxamer 234. In some embodiments, a suitable poloxamer is poloxamer 235. In some embodiments, a suitable poloxamer is poloxamer 237. In some embodiments, a suitable poloxamer is poloxamer 238. In some embodiments, a suitable poloxamer is poloxamer 282. In some embodiments, a suitable poloxamer is poloxamer 284. In some embodiments, a suitable poloxamer is poloxamer 288. In some embodiments, a suitable poloxamer is poloxamer 304. In some embodiments, a suitable poloxamer is poloxamer 331. In some embodiments, a suitable poloxamer is poloxamer 333. In some embodiments, a suitable poloxamer is poloxamer 334. In some embodiments, a suitable poloxamer is poloxamer 335. In some embodiments, a suitable poloxamer is poloxamer 338. In some embodiments, a suitable poloxamer is poloxamer 401. In some embodiments, a suitable poloxamer is poloxamer 402. In some embodiments, a suitable poloxamer is poloxamer 403. In some embodiments, a suitable poloxamer is poloxamer 407. In some embodiments, a suitable poloxamer is a combination thereof.

In some embodiments, a suitable poloxamer has an average molecular weight of about 4,000 g/mol to about 20,000 g/mol. In some embodiments, a suitable poloxamer has an average molecular weight of about 1,000 g/mol to about 50,000 g/mol. In some embodiments, a suitable poloxamer has an average molecular weight of about 1,000 g/mol. In some embodiments, a suitable poloxamer has an average molecular weight of about 2,000 g/mol. In some embodiments, a suitable poloxamer has an average molecular weight of about 3,000 g/mol. In some embodiments, a suitable poloxamer has an average molecular weight of about 4,000 g/mol. In some embodiments, a suitable poloxamer has an average molecular weight of about 5,000 g/mol. In some embodiments, a suitable poloxamer has an average molecular weight of about 6,000 g/mol. In some embodiments, a suitable poloxamer has an average molecular weight of about 7,000 g/mol. In some embodiments, a suitable poloxamer has an average molecular weight of about 8,000 g/mol. In some embodiments, a suitable poloxamer has an average molecular weight of about 9,000 g/mol. In some embodiments, a suitable poloxamer has an average molecular weight of about 10,000 g/mol. In some embodiments, a suitable poloxamer has an average molecular weight of about 20,000 g/mol. In some embodiments, a suitable poloxamer has an average molecular weight of about 25,000 g/mol. In some embodiments, a suitable poloxamer has an average molecular weight of about 30,000 g/mol. In some embodiments, a suitable poloxamer has an average molecular weight of about 40,000 g/mol. In some embodiments, a suitable poloxamer has an average molecular weight of about 50,000 g/mol.

Other Amphiphilic Polymers

In some embodiments, an amphiphilic polymer is a poloxamine, e.g., tetronic 304 or tetronic 904.

In some embodiments, an amphiphilic polymer is a polyvinylpyrrolidone (PVP), such as PVP with molecular weight of 3 kDa, 10 kDa, or 29 kDa.

In some embodiments, an amphiphilic polymer is a polyethylene glycol ether (Brij), polysorbate, sorbitan, and derivatives thereof. In some embodiments, an amphiphilic polymer is a polysorbate, such as PS 20.

In some embodiments, an amphiphilic polymer is polyethylene glycol ether (Brij), poloxamer, polysorbate, sorbitan, or derivatives thereof.

In some embodiments, an amphiphilic polymer is a polyethylene glycol ether. In some embodiments, a suitable polyethylene glycol ether is a compound of Formula (S-I):

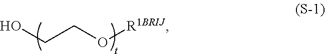

or a salt or isomer thereof, wherein:
t is an integer between 1 and 100;
R^{1BRU} independently is C₁₀₋₄₀ alkyl, C₁₀₋₄₀ alkenyl, or C₁₀₋₄₀ alkynyl; and optionally one or more methylene groups of R^{5PEG} are independently replaced with C₃₋₁₀ carbocyclylene, 4 to 10 membered heterocyclylene, C₆₋₁₀ arylene, 4 to 10 membered heteroarylene, —N(R^{N})—, —O—, —S—, —C(O)—, —C(O)N(R^{N})—, —NR^{N}C(O)—, —NRC(O)N(R)—, —C(O)O— —OC(O)—, —OC(O)O— —OC(O)N(R^{N})—, —NRNC(O)O— —C(O)S— —SC(O)—, —C(═NR^{N})—, —C(═NR)N(R)—, —NRNC(═NR^{N})— —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O— —OS(O)O— —OS(O)₂— —S(O)₂O— —OS(O)₂O— —N(R^{N})S(O)—, —S(O)N(R^{N})——N(R^{N})S(O)N(R^{N})— —OS(O)N(R^{N})— —N(R^{N})S(O)O— —S(O)₂— —N(R^{N})S(O)₂— —S(O)₂N(R^{N})—, —N(R^{N})S(O)₂N(R^{N})— —OS(O)₂N(R^{N})— or —N(R^{N})S(O)₂O—; and
each instance of R^{N} is independently hydrogen, C₁₋₆ alkyl, or a nitrogen protecting group.

In some embodiments, R^{1BRU} is C is alkyl. For example, the polyethylene glycol ether is a compound of Formula (S-Ia):

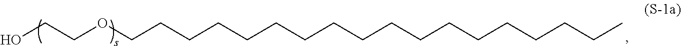

or a salt or isomer thereof, wherein s is an integer between 1 and 100.

In some embodiments, R^{1BRU} is C is alkenyl. For example, a suitable polyethylene glycol ether is a compound of Formula (S-Ib):

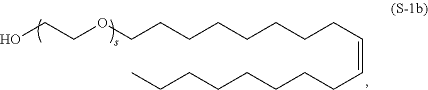

or a salt or isomer thereof, wherein s is an integer between 1 and 100.

Typically, an amphiphilic polymer (e.g., a poloxamer) is present in a formulation at an amount lower than its critical micelle concentration (CMC). In some embodiments, an amphiphilic polymer (e.g., a poloxamer) is present in the mixture at an amount about 1%, about 2%, about 3%, about 4%, about 5%, about 6%, about 7%, about 8%, about 9%, about 10%, about 15%, about 20%, about 25%, about 30%, about 35%, about 40%, about 45%, or about 50% lower than its CMC. In some embodiments, an amphiphilic polymer (e.g., a poloxamer) is present in the mixture at an amount about 0.9%, 0.8%, 0.7%, 0.6%, 0.5%, 0.4%, 0.3%, 0.2%, 0.1% lower than its CMC. In some embodiments, an amphiphilic polymer (e.g., a poloxamer) is present in the mixture at an amount about 55%, 60%, 65%, 70%, 75%, 80%, 90%, or 95% lower than its CMC.

In some embodiments, less than about 0.1%, 0.09%, 0.08%, 0.07%, 0.06%, 0.05%, 0.04%, 0.03%, 0.02%, or 0.01% of the original amount of the amphiphilic polymer (e.g., the poloxamer) present in the formulation remains upon removal. In some embodiments, a residual amount of the amphiphilic polymer (e.g., the poloxamer) remains in a formulation upon removal. As used herein, a residual amount means a remaining amount after substantially all of the substance (an amphiphilic polymer described herein such as a poloxamer) in a composition is removed. A residual amount may be detectable using a known technique qualitatively or quantitatively. A residual amount may not be detectable using a known technique.

In some embodiments, a suitable delivery vehicle comprises less than 5% amphiphilic block copolymers (e.g., poloxamers). In some embodiments, a suitable delivery vehicle comprises less than 3% amphiphilic block copolymers (e.g., poloxamers). In some embodiments, a suitable delivery vehicle comprises less than 2.5% amphiphilic block copolymers (e.g., poloxamers). In some embodiments, suitable delivery vehicle comprises less than 2% amphiphilic block copolymers (e.g., poloxamers). In some embodiments, a suitable delivery vehicle comprises less than 1.5% amphiphilic block copolymers (e.g., poloxamers). In some embodiments, a suitable delivery vehicle comprises less than 1% amphiphilic block copolymers (e.g., poloxamers). In some embodiments, a suitable delivery vehicle comprises less than 0.5% (e.g., less than 0.4%, 0.3%, 0.2%, 0.1%) amphiphilic block copolymers (e.g., poloxamers). In some embodiments, a suitable delivery vehicle comprises less than 0.09%, 0.08%, 0.07%, 0.06%, 0.05%, 0.04%, 0.03%, 0.02%, or 0.01% amphiphilic block copolymers (e.g., poloxamers). In some embodiments, a suitable delivery vehicle comprises less than 0.01% amphiphilic block copolymers (e.g., poloxamers). In some embodiments, a suitable delivery vehicle contains a residual amount of amphiphilic polymers (e.g., poloxamers). As used herein, a residual amount means a remaining amount after substantially all of the substance (an amphiphilic polymer described herein such as a poloxamer) in a composition is removed. A residual amount may be detectable using a known technique qualitatively or quantitatively. A residual amount may not be detectable using a known technique.

Polymers

In some embodiments, a suitable delivery vehicle is formulated using a polymer as a carrier, alone or in combination with other carriers including various lipids described herein. Thus, in some embodiments, liposomal delivery vehicles, as used herein, also encompass nanoparticles comprising polymers. Suitable polymers may include, for example, polyacrylates, polyalkycyanoacrylates, polylactide, polylactide-polyglycolide copolymers, polycaprolactones, dextran, albumin, gelatin, alginate, collagen, chitosan, cyclodextrins, protamine, PEGylated protamine, PLL, PEGylated PLL and polyethylenimine (PEI). When PEI is present, it may be branched PEI of a molecular weight ranging from 10 to 40 kDa, e.g., 25 kDa branched PEI (Sigma #408727).

According to various embodiments, the selection of cationic lipids, non-cationic lipids, PEG-modified lipids, cholesterol-based lipids, and/or amphiphilic block copolymers which comprise the lipid nanoparticle, as well as the relative molar ratio of such components (lipids) to each other, is based upon the characteristics of the selected lipid(s), the nature of the intended target cells, the characteristics of the nucleic acid to be delivered. Additional considerations include, for example, the saturation of the alkyl chain, as well as the size, charge, pH, pKa, fusogenicity and toxicity of the selected lipid(s). Thus, the molar ratios may be adjusted accordingly.

Liposomal Compositions

Liposomal compositions that are suitable for the delivery of mRNA to target cells in vivo may include the compounds of the invention as a cationic lipid component. In some embodiments, the ratio of cationic lipid(s) to non-cationic lipid(s) to cholesterol-based lipid(s) to PEG-modified lipid(s) may be between about 30-60:25-35:20-30:1-15, respectively. In some embodiments, the ratio of cationic lipid(s) to non-cationic lipid(s) to cholesterol-based lipid(s) to PEG-modified lipid(s) is approximately 40:30:20:10, respectively. In some embodiments, the ratio of cationic lipid(s) to non-cationic lipid(s) to cholesterol-based lipid(s) to PEG-modified lipid(s) is approximately 40:30:25:5, respectively. In some embodiments, the ratio of cationic lipid(s) to non-cationic lipid(s) to cholesterol-based lipid(s) to PEG-modified lipid(s) is approximately 40:32:25:3, respectively. In some embodiments, the ratio of cationic lipid(s) to non-cationic lipid(s) to cholesterol-based lipid(s) to PEG-modified lipid(s) is approximately 50:25:20:5. In some embodiments, the ratio of sterol lipid(s) to non-cationic lipid(s) to PEG-modified lipid(s) is 50:45:5. In some embodiments, the ratio of sterol lipid(s) to non-cationic lipid(s) to PEG-modified lipid(s) is 50:40:10. In some embodiments, the ratio of sterol lipid(s) to non-cationic lipid(s) to PEG-modified lipid(s) is 55:40:5. In some embodiments, the ratio of sterol lipid(s) to non-cationic lipid(s) to PEG-modified lipid(s) is 55:35:10. In some embodiments, the ratio of sterol lipid(s) to non-cationic lipid(s) to PEG-modified lipid(s) is 60:35:5. In some embodiments, the ratio of sterol lipid(s) to non-cationic lipid(s) to PEG-modified lipid(s) is 60:30:10.

Exemplary liposomal compositions include a compound of the invention as the sole cationic lipid component. A suitable liposomal composition may further comprise cholesterol, a non-cationic lipid such as DOPE, and a PEG-modified lipid such as DMG-PEG2K.

Ratio of Distinct Lipid Components

A suitable liposome for the present invention may include one or more of any of the cationic lipids, non-cationic lipids, cholesterol lipids, PEG-modified lipids, amphiphilic block copolymers and/or polymers described herein at various ratios. In some embodiments, a lipid nanoparticle comprises five and no more than five distinct components of nanoparticle. In some embodiments, a lipid nanoparticle comprises four and no more than four distinct components of nanoparticle. In some embodiments, a lipid nanoparticle comprises three and no more than three distinct components of nanoparticle. As non-limiting example, a suitable liposome formulation may include a combination of the following lipid components: a compound of Formula (A′), (A), (I), (I-a), (I-a), (I-b), (I-b′), (I-c), (I-c′), (I-c-1), (I-c′-1), (I-c-2), (I-c′-2), (I-d), (I-d′), (I-d-1), (I-d-2), (I-e), (I-e′), (I-e-1), (I-e-2), (I-f), (I-f), (II), (II-a), (II-a′), (III), (III′), (III-a), (III-a′), (III-b), (III-b′), (III-c), (III-c-1), (III-c′-1), (III-c-2), (III-c′-2), (III-c′), (III-d), (III-d′), (III-d-1), (III-d-2), (III-e), (III-e′), (III-e-1), (III-e-2), (III-f), (III-f′), (IV), (IV-a), or (IV-a′), such as any of Compounds 1-552, as the cationic lipid component, DOPE or DEPE as the non-cationic lipid component, cholesterol as the cholesterol-based lipid, and DMG-PEG2K as the PEG-modified lipid.

In various embodiments, cationic lipids (e.g., a compound of Formula (A′), (A), (I), (I-a), (I-a′), (I-b), (I-b′), (I-c), (I-c′), (I-c-1), (I-c′-1), (I-c-2), (I-c′-2), (I-d), (I-d′), (I-d-1), (I-d-2), (I-e), (I-e′), (I-e-1), (I-e-2), (I-f), (I-f′), (II), (II-a), (II-a′), (III), (III′), (III-a), (III-a′), (III-b), (III-b′), (III-c), (III-c-1), (III-c′-1), (III-c-2), (III-c′-2), (III-c′), (III-d), (III-d′), (III-d-1), (III-d-2), (III-e), (III-e′), (III-e-1), (III-e-2), (III-f), (III-f′), (IV), (IV-a), or (IV-a′), such as any of Compounds 1-552) constitute about 30-60% (e.g., about 30-55%, about 30-50%, about 30-45%, about 30-40%, about 35-50%, about 35-45%, or about 35-40%) of the liposome by molar ratio. In some embodiments, the percentage of cationic lipids (e.g., a compound of Formula (A′), (A), (I), (I-a), (I-a′), (I-b), (I-b′), (I-c), (I-c′), (I-c-1), (I-c′-1), (I-c-2), (I-c′-2), (I-d), (I-d′), (I-d-1), (I-d-2), (I-e), (I-e′), (I-e-1), (I-e-2), (I-f), (I-f), (II), (II-a), (II-a′), (III), (III′), (III-a), (III-a′), (III-b), (III-b′), (III-c), (III-c-1), (III-c′-1), (III-c-2), (III-c′-2), (III-c′), (III-d), (III-d′), (III-d-1), (III-d-2), (III-e), (III-e′), (III-e-1), (III-e-2), (III-f), (III-f′), (IV), (IV-a), or (IV-a′), such as any of Compounds 1-552) is or greater than about 30%, about 35%, about 40%, about 45%, about 50%, about 55%, or about 60% of the liposome by molar ratio.

In some embodiments, the molar ratio of cationic lipid(s) to non-cationic lipid(s) to cholesterol-based lipid(s) to PEG-modified lipid(s) may be between about 30-60:25-35:20-30:1-15, respectively. In some embodiments, the ratio of cationic lipid(s) to non-cationic lipid(s) to cholesterol-based lipid(s) to PEG-modified lipid(s) is approximately 40:30:20:10, respectively. In some embodiments, the ratio of cationic lipid(s) to non-cationic lipid(s) to cholesterol-based lipid(s) to PEG-modified lipid(s) is approximately 40:30:25:5, respectively. In some embodiments, the ratio of cationic lipid(s) to non-cationic lipid(s) to cholesterol-based lipid(s) to PEG-modified lipid(s) is approximately 40:32:25:3, respectively. In some embodiments, the ratio of cationic lipid(s) to non-cationic lipid(s) to cholesterol-based lipid(s) to PEG-modified lipid(s) is approximately 50:25:20:5.

Formation of Liposomes Encapsulating mRNA

The liposomal transfer vehicles for use in the compositions of the invention can be prepared by various techniques which are presently known in the art. For example, multilamellar vesicles (MLV) may be prepared according to conventional techniques, such as by depositing a selected lipid on the inside wall of a suitable container or vessel by dissolving the lipid in an appropriate solvent, and then evaporating the solvent to leave a thin film on the inside of the vessel or by spray drying. An aqueous phase may then be added to the vessel with a vortexing motion which results in the formation of MLVs. Unilamellar vesicles (ULV) can then be formed by homogenization, sonication or extrusion of the multilamellar vesicles. In addition, unilamellar vesicles can be formed by detergent removal techniques.

Various methods are described in published U.S. Application No. US 2011/0244026, published U.S. Application No. US 2016/0038432, published U.S. Application No. US 2018/0153822, published U.S. Application No. US 2018/0125989 and U.S. Provisional Application No. 62/877,597, filed Jul. 23, 2019 and can be used to practice the present invention, all of which are incorporated herein by reference. As used herein, Process A refers to a conventional method of encapsulating mRNA by mixing mRNA with a mixture of lipids, without first pre-forming the lipids into lipid nanoparticles, as described in US 2016/0038432. As used herein, Process B refers to a process of encapsulating messenger RNA (mRNA) by mixing pre-formed lipid nanoparticles with mRNA, as described in US 2018/0153822.

Briefly, the process of preparing mRNA-loaded lipid liposomes includes a step of heating one or more of the solutions (i.e., applying heat from a heat source to the solution) to a temperature (or to maintain at a temperature) greater than ambient temperature, the one more solutions being the solution comprising the pre-formed lipid nanoparticles, the solution comprising the mRNA and the mixed solution comprising the lipid nanoparticle encapsulated mRNA. In some embodiments, the process includes the step of heating one or both of the mRNA solution and the pre-formed lipid nanoparticle solution, prior to the mixing step. In some embodiments, the process includes heating one or more one or more of the solution comprising the pre-formed lipid nanoparticles, the solution comprising the mRNA and the solution comprising the lipid nanoparticle encapsulated mRNA, during the mixing step. In some embodiments, the process includes the step of heating the lipid nanoparticle encapsulated mRNA, after the mixing step. In some embodiments, the temperature to which one or more of the solutions is heated (or at which one or more of the solutions is maintained) is or is greater than about 30° C., 37° C., 40° C., 45° C., 50° C., 55° C., 60° C., 65° C., or 70° C. In some embodiments, the temperature to which one or more of the solutions is heated ranges from about 25-70° C., about 30-70° C., about 35-70° C., about 40-70° C., about 45-70° C., about 50-70° C., or about 60-70° C. In some embodiments, the temperature greater than ambient temperature to which one or more of the solutions is heated is about 65° C.

Various methods may be used to prepare an mRNA solution suitable for the present invention. In some embodiments, mRNA may be directly dissolved in a buffer solution described herein. In some embodiments, an mRNA solution may be generated by mixing an mRNA stock solution with a buffer solution prior to mixing with a lipid solution for encapsulation. In some embodiments, an mRNA solution may be generated by mixing an mRNA stock solution with a buffer solution immediately before mixing with a lipid solution for encapsulation. In some embodiments, a suitable mRNA stock solution may contain mRNA in water at a concentration at or greater than about 0.2 mg/ml, 0.4 mg/ml, 0.5 mg/ml, 0.6 mg/ml, 0.8 mg/ml, 1.0 mg/ml, 1.2 mg/ml, 1.4 mg/ml, 1.5 mg/ml, or 1.6 mg/ml, 2.0 mg/ml, 2.5 mg/ml, 3.0 mg/ml, 3.5 mg/ml, 4.0 mg/ml, 4.5 mg/ml, or 5.0 mg/ml.

In some embodiments, an mRNA stock solution is mixed with a buffer solution using a pump. Exemplary pumps include but are not limited to gear pumps, peristaltic pumps and centrifugal pumps.

Typically, the buffer solution is mixed at a rate greater than that of the mRNA stock solution. For example, the buffer solution may be mixed at a rate at least 1×, 2×, 3×, 4×, 5×, 6×, 7×, 8×, 9×, 10×, 15×, or 20× greater than the rate of the mRNA stock solution. In some embodiments, a buffer solution is mixed at a flow rate ranging between about 100-6000 ml/minute (e.g., about 100-300 ml/minute, 300-600 ml/minute, 600-1200 ml/minute, 1200-2400 ml/minute, 2400-3600 ml/minute, 3600-4800 ml/minute, 4800-6000 ml/minute, or 60-420 ml/minute). In some embodiments, a buffer solution is mixed at a flow rate of or greater than about 60 ml/minute, 100 ml/minute, 140 ml/minute, 180 ml/minute, 220 ml/minute, 260 ml/minute, 300 ml/minute, 340 ml/minute, 380 ml/minute, 420 ml/minute, 480 ml/minute, 540 ml/minute, 600 ml/minute, 1200 ml/minute, 2400 ml/minute, 3600 ml/minute, 4800 ml/minute, or 6000 ml/minute.

In some embodiments, an mRNA stock solution is mixed at a flow rate ranging between about 10-600 ml/minute (e.g., about 5-50 ml/minute, about 10-30 ml/minute, about 30-60 ml/minute, about 60-120 ml/minute, about 120-240 ml/minute, about 240-360 ml/minute, about 360-480 ml/minute, or about 480-600 ml/minute). In some embodiments, an mRNA stock solution is mixed at a flow rate of or greater than about 5 ml/minute, 10 ml/minute, 15 ml/minute, 20 ml/minute, 25 ml/minute, 30 ml/minute, 35 ml/minute, 40 ml/minute, 45 ml/minute, 50 ml/minute, 60 ml/minute, 80 ml/minute, 100 ml/minute, 200 ml/minute, 300 ml/minute, 400 ml/minute, 500 ml/minute, or 600 ml/minute.

According to the present invention, a lipid solution contains a mixture of lipids suitable to form lipid nanoparticles for encapsulation of mRNA. In some embodiments, a suitable lipid solution is ethanol based. For example, a suitable lipid solution may contain a mixture of desired lipids dissolved in pure ethanol (i.e., 100% ethanol). In another embodiment, a suitable lipid solution is isopropyl alcohol based. In another embodiment, a suitable lipid solution is dimethylsulfoxide-based. In another embodiment, a suitable lipid solution is a mixture of suitable solvents including, but not limited to, ethanol, isopropyl alcohol and dimethylsulfoxide.

A suitable lipid solution may contain a mixture of desired lipids at various concentrations. For example, a suitable lipid solution may contain a mixture of desired lipids at a total concentration of or greater than about 0.1 mg/ml, 0.5 mg/ml, 1.0 mg/ml, 2.0 mg/ml, 3.0 mg/ml, 4.0 mg/ml, 5.0 mg/ml, 6.0 mg/ml, 7.0 mg/ml, 8.0 mg/ml, 9.0 mg/ml, 10 mg/ml, 15 mg/ml, 20 mg/ml, 30 mg/ml, 40 mg/ml, 50 mg/ml, or 100 mg/ml. In some embodiments, a suitable lipid solution may contain a mixture of desired lipids at a total concentration ranging from about 0.1-100 mg/ml, 0.5-90 mg/ml, 1.0-80 mg/ml, 1.0-70 mg/ml, 1.0-60 mg/ml, 1.0-50 mg/ml, 1.0-40 mg/ml, 1.0-30 mg/ml, 1.0-20 mg/ml, 1.0-15 mg/ml, 1.0-10 mg/ml, 1.0-9 mg/ml, 1.0-8 mg/ml, 1.0-7 mg/ml, 1.0-6 mg/ml, or 1.0-5 mg/ml. In some embodiments, a suitable lipid solution may contain a mixture of desired lipids at a total concentration up to about 100 mg/ml, 90 mg/ml, 80 mg/ml, 70 mg/ml, 60 mg/ml, 50 mg/ml, 40 mg/ml, 30 mg/ml, 20 mg/ml, or 10 mg/ml.

Any desired lipids may be mixed at any ratios suitable for encapsulating mRNAs. In some embodiments, a suitable lipid solution contains a mixture of desired lipids including cationic lipids, helper lipids (e.g. non cationic lipids and/or cholesterol lipids), amphiphilic block copolymers (e.g. poloxamers) and/or PEGylated lipids. In some embodiments, a suitable lipid solution contains a mixture of desired lipids including one or more cationic lipids, one or more helper lipids (e.g. non cationic lipids and/or cholesterol lipids) and one or more PEGylated lipids.

In certain embodiments, provided compositions comprise a liposome wherein the mRNA is associated on both the surface of the liposome and encapsulated within the same liposome. For example, during preparation of the compositions of the present invention, cationic liposomes may associate with the mRNA through electrostatic interactions.

In some embodiments, the compositions and methods of the invention comprise mRNA encapsulated in a liposome. In some embodiments, the one or more mRNA species may be encapsulated in the same liposome. In some embodiments, the one or more mRNA species may be encapsulated in different liposomes. In some embodiments, the mRNA is encapsulated in one or more liposomes, which differ in their lipid composition, molar ratio of lipid components, size, charge (zeta potential), targeting ligands and/or combinations thereof. In some embodiments, the one or more liposome may have a different composition of sterol-based cationic lipids, neutral lipid, PEG-modified lipid and/or combinations thereof. In some embodiments the one or more liposomes may have a different molar ratio of cholesterol-based cationic lipid, neutral lipid, and PEG-modified lipid used to create the liposome.

The process of incorporation of a desired nucleic acid (e.g., mRNA) into a liposome is often referred to as “loading”. Exemplary methods are described in Lasic, et al. FEBS Lett., 312: 255-258, 1992, which is incorporated herein by reference. The liposome-incorporated nucleic acids may be completely or partially located in the interior space of the liposome, within the bilayer membrane of the liposome, or associated with the exterior surface of the liposome membrane. The incorporation of a nucleic acid into liposomes is also referred to herein as “encapsulation” wherein the nucleic acid is entirely contained within the interior space of the liposome. The purpose of incorporating an mRNA into a transfer vehicle, such as a liposome, is often to protect the nucleic acid from an environment which may contain enzymes or chemicals that degrade nucleic acids and/or systems or receptors that cause the rapid excretion of the nucleic acids. Accordingly, in some embodiments, a suitable delivery vehicle is capable of enhancing the stability of the mRNA contained therein and/or facilitate the delivery of therapeutic agent (e.g., mRNA) to the target cell or tissue.

Suitable liposomes in accordance with the present invention may be made in various sizes. In some embodiments, provided liposomes may be made smaller than previously known liposomes. In some embodiments, decreased size of liposomes is associated with more efficient delivery of therapeutic agent (e.g., mRNA). Selection of an appropriate liposome size may take into consideration the site of the target cell or tissue and to some extent the application for which the liposome is being made.

In some embodiments, an appropriate size of liposome is selected to facilitate systemic distribution of antibody encoded by the mRNA. In some embodiments, it may be desirable to limit transfection of the mRNA to certain cells or tissues. For example, to target hepatocytes a liposome may be sized such that its dimensions are smaller than the fenestrations of the endothelial layer lining hepatic sinusoids in the liver; in such cases the liposome could readily penetrate such endothelial fenestrations to reach the target hepatocytes.

Alternatively or additionally, a liposome may be sized such that the dimensions of the liposome are of a sufficient diameter to limit or expressly avoid distribution into certain cells or tissues.

A variety of alternative methods known in the art are available for sizing of a population of liposomes. One such sizing method is described in U.S. Pat. No. 4,737,323, incorporated herein by reference. Sonicating a liposome suspension either by bath or probe sonication produces a progressive size reduction down to small ULV less than about 0.05 microns in diameter. Homogenization is another method that relies on shearing energy to fragment large liposomes into smaller ones. In a typical homogenization procedure, MLV are recirculated through a standard emulsion homogenizer until selected liposome sizes, typically between about 0.1 and 0.5 microns, are observed. The size of the liposomes may be determined by quasi-electric light scattering (QELS) as described in Bloomfield, Ann. Rev. Biophys. Bioeng., 10:421-450 (1981), incorporated herein by reference. Average liposome diameter may be reduced by sonication of formed liposomes. Intermittent sonication cycles may be alternated with QELS assessment to guide efficient liposome synthesis.

Provided Nanoparticles Encapsulating mRNA

In some embodiments, majority of purified nanoparticles in a composition, i.e., greater than about 50%, 55%, 60%, 65%, 70%, 75%, 80%, 85%, 90%, 95%, 96%, 97%, 98%, or 99% of the nanoparticles, have a size of about 150 nm (e.g., about 145 nm, about 140 nm, about 135 nm, about 130 nm, about 125 nm, about 120 nm, about 115 nm, about 110 nm, about 105 nm, about 100 nm, about 95 nm, about 90 nm, about 85 nm, or about 80 nm). In some embodiments, substantially all of the purified nanoparticles have a size of about 150 nm (e.g., about 145 nm, about 140 nm, about 135 nm, about 130 nm, about 125 nm, about 120 nm, about 115 nm, about 110 nm, about 105 nm, about 100 nm, about 95 nm, about 90 nm, about 85 nm, or about 80 nm).

In some embodiments, a lipid nanoparticle has an average size of less than 150 nm. In some embodiments, a lipid nanoparticle has an average size of less than 120 nm. In some embodiments, a lipid nanoparticle has an average size of less than 100 nm. In some embodiments, a lipid nanoparticle has an average size of less than 90 nm. In some embodiments, a lipid nanoparticle has an average size of less than 80 nm. In some embodiments, a lipid nanoparticle has an average size of less than 70 nm. In some embodiments, a lipid nanoparticle has an average size of less than 60 nm. In some embodiments, a lipid nanoparticle has an average size of less than 50 nm. In some embodiments, a lipid nanoparticle has an average size of less than 30 nm. In some embodiments, a lipid nanoparticle has an average size of less than 20 nm.

In some embodiments, greater than about 70%, 75%, 80%, 85%, 90%, 95%, 96%, 97%, 98%, 99% of the lipid nanoparticles (e.g., liposomes) in a composition provided by the present invention have a size ranging from about 70-120 nm (e.g., about 75-115 nm, about 80-110 nm, or about 85-105 nm). In some embodiments, substantially all of the lipid nanoparticles (e.g., liposomes) have a size ranging from about 70-150 nm (e.g., about 80-130 nm or about 90-120 nm). Compositions with lipid nanoparticles (e.g., liposomes) having an average size of about 90-130 nm are particular suitable for liver delivery via intravenous administration as well as pulmonary delivery via aerosol administration (e.g., via nebulization).

In some embodiments, the dispersity, or measure of heterogeneity in size of molecules (PDI), of nanoparticles in a composition provided by the present invention is less than about 0.5. In some embodiments, a lipid nanoparticle has a PDI of less than about 0.5. In some embodiments, a lipid nanoparticle has a PDI of less than about 0.4. In some embodiments, a lipid nanoparticle has a PDI of less than about 0.3. In some embodiments, a lipid nanoparticle has a PDI of less than about 0.28. In some embodiments, a lipid nanoparticle has a PDI of less than about 0.25. In some embodiments, a lipid nanoparticle has a PDI of less than about 0.23. In some embodiments, a lipid nanoparticle has a PDI of less than about 0.20. In some embodiments, a lipid nanoparticle has a PDI of less than about 0.18. In some embodiments, a lipid nanoparticle has a PDI of less than about 0.16. In some embodiments, a lipid nanoparticle has a PDI of less than about 0.14. In some embodiments, a lipid nanoparticle has a PDI of less than about 0.12. In some embodiments, a lipid nanoparticle has a PDI of less than about 0.10. In some embodiments, a lipid nanoparticle has a PDI of less than about 0.08. Typical lipid nanoparticles for use with the present invention have a PDI of less than about 0.20.

In some embodiments, greater than about 75%, 80%, 85%, 90%, 95%, 96%, 97%, 98%, or 99% of the purified lipid nanoparticles in a composition provided by the present invention encapsulate an mRNA within each individual particle. In some embodiments, substantially all of the purified lipid nanoparticles in a composition encapsulate an mRNA within each individual particle. In some embodiments, a lipid nanoparticle has an encapsulation efficiency of between 50% and 99%. In some embodiments, a lipid nanoparticle has an encapsulation efficiency of greater than about 60%. In some embodiments, a lipid nanoparticle has an encapsulation efficiency of greater than about 65%. In some embodiments, a lipid nanoparticle has an encapsulation efficiency of greater than about 70%. In some embodiments, a lipid nanoparticle has an encapsulation efficiency of greater than about 75%. In some embodiments, a lipid nanoparticle has an encapsulation efficiency of greater than about 80%. In some embodiments, a lipid nanoparticle has an encapsulation efficiency of greater than about 85%. In some embodiments, a lipid nanoparticle has an encapsulation efficiency of greater than about 90%. In some embodiments, a lipid nanoparticle has an encapsulation efficiency of greater than about 92%. In some embodiments, a lipid nanoparticle has an encapsulation efficiency of greater than about 95%. In some embodiments, a lipid nanoparticle has an encapsulation efficiency of greater than about 98%. In some embodiments, a lipid nanoparticle has an encapsulation efficiency of greater than about 99%. Typically, lipid nanoparticles for use with the invention have an encapsulation efficiency of at least 65%-97%. Lipid nanoparticles with an encapsulation efficiency of greater than 80%, e.g. greater than 85% or greater than 90% are particularly suitable fro therapeutic applications.

In some embodiments, a lipid nanoparticle has a N/P ratio of between 1 and 10. As used herein, the term “N/P ratio” refers to a molar ratio of positively charged molecular units in the cationic lipids in a lipid nanoparticle relative to negatively charged molecular units in the mRNA encapsulated within that lipid nanoparticle. As such, N/P ratio is typically calculated as the ratio of moles of amine groups in cationic lipids in a lipid nanoparticle relative to moles of phosphate groups in mRNA encapsulated within that lipid nanoparticle. In some embodiments, a lipid nanoparticle has a N/P ratio above 1. In some embodiments, a lipid nanoparticle has a N/P ratio of about 1. In some embodiments, a lipid nanoparticle has a N/P ratio of about 2. In some embodiments, a lipid nanoparticle has a N/P ratio of about 3. In some embodiments, a lipid nanoparticle has a N/P ratio of about 4. In some embodiments, a lipid nanoparticle has a N/P ratio of about 5. In some embodiments, a lipid nanoparticle has a N/P ratio of about 6. In some embodiments, a lipid nanoparticle has a N/P ratio of about 7. In some embodiments, a lipid nanoparticle has a N/P ratio of about 8. A typical lipid nanoparticle for use with the invention has an N/P ratio of about 4.

In some embodiments, a composition according to the present invention contains at least about 0.5 mg, 1 mg, 5 mg, 10 mg, 100 mg, 500 mg, or 1000 mg of encapsulated mRNA. In some embodiments, a composition contains about 0.1 mg to 1000 mg of encapsulated mRNA. In some embodiments, a composition contains at least about 0.5 mg of encapsulated mRNA. In some embodiments, a composition contains at least about 0.8 mg of encapsulated mRNA. In some embodiments, a composition contains at least about 1 mg of encapsulated mRNA. In some embodiments, a composition contains at least about 5 mg of encapsulated mRNA. In some embodiments, a composition contains at least about 8 mg of encapsulated mRNA. In some embodiments, a composition contains at least about 10 mg of encapsulated mRNA. In some embodiments, a composition contains at least about 50 mg of encapsulated mRNA. In some embodiments, a composition contains at least about 100 mg of encapsulated mRNA. In some embodiments, a composition contains at least about 500 mg of encapsulated mRNA. In some embodiments, a composition contains at least about 1000 mg of encapsulated mRNA.

### Pharmaceutical Formulations and Therapeutic Uses

Compounds described herein (e.g., a compound of Formula (A′), (A), (I), (I-a), (I-a′), (I-b), (I-b′), (I-c), (I-c′), (I-c-1), (I-c′-1), (I-c-2), (I-c′-2), (I-d), (I-d′), (I-d-1), (I-d-2), (I-e), (I-e′), (I-e-1), (I-e-2), (I-f), (I-f′), (II), (II-a), (II-a′), (III), (III′), (III-a), (III-a′), (III-b), (III-b′), (III-c), (III-c-1), (III-c′-1), (III-c-2), (III-c′-2), (III-c′), (III-d), (III-d′), (III-d-1), (III-d-2), (III-e), (III-e′), (III-e-1), (III-e-2), (III-f), (III-f′), (IV), (IV-a), or (IV-a′) such as any of Compounds 1-552) may be used in the preparation of compositions (e.g., to construct liposomal compositions) that facilitate or enhance the delivery and release of encapsulated materials (e.g., one or more therapeutic polynucleotides) to one or more target cells (e.g., by permeating or fusing with the lipid membranes of such target cells).

For example, when a liposomal composition (e.g., a lipid nanoparticle) comprises or is otherwise enriched with one or more of the compounds disclosed herein, the phase transition in the lipid bilayer of the one or more target cells may facilitate the delivery of the encapsulated materials (e.g., one or more therapeutic polynucleotides encapsulated in a lipid nanoparticle) into the one or more target cells.

Similarly, in certain embodiments compounds described herein (e.g., a compound of Formula (A′), (A), (1), (I-a), (I-a′), (I-b), (I-b′), (I-c), (I-c′), (I-c-1), (I-c′-1), (I-c-2), (I-c′-2), (I-d), (I-d′), (I-d-1), (I-d-2), (I-e), (I-e′), (I-e-1), (I-e-2), (I-f), (I-f′), (II), (II-a), (II-a′), (III), (III′), (III-a), (III-a′), (III-b), (III-b′), (III-c), (III-c-1), (III-c′-1), (III-c-2), (III-c′-2), (III-c′), (III-d), (III-d′), (III-d-1), (III-d-2), (III-e), (III-e′), (III-e-1), (III-e-2), (III-f), (III-f′), (IV), (IV-a), or (IV-a′) such as any of Compounds 1-552) may be used to prepare liposomal vehicles that are characterized by their reduced toxicity in vivo. In certain embodiments, the reduced toxicity is a function of the high transfection efficiencies associated with the compositions disclosed herein, such that a reduced quantity of such composition may administered to the subject to achieve a desired therapeutic response or outcome.

Thus, pharmaceutical formulations comprising a compound described (e.g., a compound of Formula (A′), (A), (I), (I-a), (I-a′), (I-b), (I-b′), (I-c), (I-c′), (I-c-1), (I-c′-1), (I-c-2), (I-c′-2), (I-d), (I-d′), (I-d-1), (I-d-2), (I-e), (I-e′), (I-e-1), (I-e-2), (I-f), (I-f′), (II), (II-a), (II-a′), (III), (III′), (III-a), (III-a′), (III-b), (III-b′), (III-c), (III-c-1), (III-c′-1), (III-c-2), (III-c′-2), (III-c′), (III-d), (III-d′), (III-d-1), (III-d-2), (III-e), (III-e′), (III-e-1), (III-e-2), (III-f), (III-f′), (IV), (IV-a), or (IV-a′) such as any of Compounds 1-552) and nucleic acids provided by the present invention may be used for various therapeutic purposes. To facilitate delivery of nucleic acids in vivo, a compound described herein (e.g., a compound of Formula (A′), (A), (I), (I-a), (I-a′), (I-b), (I-b′), (I-c), (I-c′), (I-c-1), (I-c′-1), (I-c-2), (I-c′-2), (d), (I-d′), (I-d-1), (I-d-2), (I-e), (I-e′), (I-e-1), (I-e-2), (I-f), (I-f′), (II), (II-a), (II-a′), (III), (III′), (III-a), (III-a′), (III-b), (III-b′), (III-c), (III-c-1), (III-c′-1), (III-c-2), (III-c′-2), (III-c′), (III-d), (III-d′), (III-d-1), (III-d-2), (III-e), (III-e′), (III-e-1), (III-e-2), (III-f), (III-f′), (IV), (IV-a), or (IV-a′) such as any of Compounds 1-552) and nucleic acids can be formulated in combination with one or more additional pharmaceutical carriers, targeting ligands or stabilizing reagents. In some embodiments, a compound described herein (e.g., a compound of Formula (A′), (A), (I), (I-a), (I-a′), (I-b), (I-b′), (I-c), (I-c′), (I-c-1), (I-c′-1), (I-c-2), (I-c′-2), (I-d), (I-d′), (I-d-1), (I-d-2), (I-e), (I-e′), (I-e-1), (I-e-2), (I-f), (I-f′), (II), (II-a), (II-a′), (III), (III′), (III-a), (III-a′), (III-b), (III-b′), (III-c), (III-c-1), (III-c′-1), (III-c-2), (III-c′-2), (III-c′), (III-d), (III-d′), (III-d-1), (III-d-2), (III-e), (III-e′), (III-e-1), (III-e-2), (III-f), (III-f′), (IV), (IV-a), or (IV-a′) such as any of Compounds 1-552) can be formulated via pre-mixed lipid solution. In other embodiments, a composition comprising a compound described herein (e.g., a compound of Formula (A′), (A), (I), (I-a), (I-a′), (I-b), (I-b′), (I-c), (I-c′), (I-c-1), (I-c′-1), (I-c-2), (I-c′-2), (I-d), (I-d′), (I-d-1), (I-d-2), (I-e), (I-e′), (I-e-1), (I-e-2), (I-f), (I-f′), (II), (II-a), (II-a′), (III), (III′), (III-a), (III-a′), (III-b), (III-b′), (III-c), (III-c-1), (III-c′-1), (III-c-2), (III-c′-2), (III-c′), (III-d), (III-d′), (III-d-1), (III-d-2), (III-e), (III-e′), (III-e-1), (III-e-2), (III-f), (III-f′), (IV), (IV-a), or (IV-a′) such as any of Compounds 1-552) can be formulated using post-insertion techniques into the lipid membrane of the nanoparticles. Techniques for formulation and administration of drugs may be found in “Remington's Pharmaceutical Sciences,” Mack Publishing Co., Easton, Pa., latest edition.

Suitable routes of administration include, for example, oral, rectal, vaginal, transmucosal, pulmonary including intratracheal or inhaled, or intestinal administration; parenteral delivery, including intradermal, transdermal (topical), intramuscular, subcutaneous, intramedullary injections, as well as intrathecal, direct intraventricular, intravenous, intraperitoneal, or intranasal. In particular embodiments, the intramuscular administration is to a muscle selected from the group consisting of skeletal muscle, smooth muscle and cardiac muscle. In some embodiments the administration results in delivery of the nucleic acids to a muscle cell. In some embodiments the administration results in delivery of the nucleic acids to a hepatocyte (i.e., liver cell).

The choice of administration route depends on the target cell or tissues. Systemic delivery of the mRNA-encoded protein or peptide may be achieved, e.g., by intravenous, intramuscular or pulmonary administration of the mRNA, typically encapsulated in a lipid nanoparticle (e.g., a liposome). Intravenous delivery can be used to efficiently target hepatocytes. Intramuscular administration is typically the method of choice for delivering mRNA encoding an immunogenic protein or peptide (e.g., as an antigen for use as a vaccine). Pulmonary delivery is commonly used to target the lung epithelium. In some embodiments, mRNA-loaded lipid nanoparticles are administered by pulmonary delivery via nebulization, typically involving a suitable nebulizing apparatus (e.g., a mesh nebulizer).

Alternatively or additionally, pharmaceutical formulations of the invention may be administered in a local rather than systemic manner, for example, via injection of the pharmaceutical formulation directly into a targeted tissue, preferably in a sustained release formulation. Local delivery can be affected in various ways, depending on the tissue to be targeted. Exemplary tissues in which delivered mRNA may be delivered and/or expressed include, but are not limited to the liver, kidney, heart, spleen, serum, brain, skeletal muscle, lymph nodes, skin, and/or cerebrospinal fluid. In embodiments, the tissue to be targeted in the liver. For example, aerosols containing compositions of the present invention can be inhaled (for nasal, tracheal, or bronchial delivery); compositions of the present invention can be injected into the site of injury, disease manifestation, or pain, for example; compositions can be provided in lozenges for oral, tracheal, or esophageal application; can be supplied in liquid, tablet or capsule form for administration to the stomach or intestines, can be supplied in suppository form for rectal or vaginal application; or can even be delivered to the eye by use of creams, drops, or even injection.

Compositions described herein can comprise mRNA encoding peptides including those described herein (e.g., a polypeptide such as a protein).

In embodiments, the mRNA encodes a polypeptide.

In embodiments, the mRNA encodes a protein.

Exemplary peptides encoded by mRNA (e.g., exemplary proteins encoded by mRNA) are described herein.

The present invention provides methods for delivering a composition having full-length mRNA molecules encoding a peptide or protein of interest for use in the treatment of a subject, e.g., a human subject or a cell of a human subject or a cell that is treated and delivered to a human subject.

Accordingly, in certain embodiments the present invention provides a method for producing a therapeutic composition comprising full-length mRNA that encodes a peptide or protein for use in the delivery to or treatment of the lung of a subject or a lung cell. In certain embodiments the present invention provides a method for producing a therapeutic composition having full-length mRNA that encodes for cystic fibrosis transmembrane conductance regulator (CFTR) protein. In certain embodiments the present invention provides a method for producing a therapeutic composition having full-length mRNA that encodes for ATP-binding cassette sub-family A member 3 protein. In certain embodiments the present invention provides a method for producing a therapeutic composition having full-length mRNA that encodes for dynein axonemal intermediate chain 1 protein. In certain embodiments the present invention provides a method for producing a therapeutic composition having full-length mRNA that encodes for dynein axonemal heavy chain 5 (DNAH5) protein. In certain embodiments the present invention provides a method for producing a therapeutic composition having full-length mRNA that encodes for alpha-1-antitrypsin protein. In certain embodiments the present invention provides a method for producing a therapeutic composition having full-length mRNA that encodes for forkhead box P3 (FOXP3) protein. In certain embodiments the present invention provides a method for producing a therapeutic composition having full-length mRNA that encodes one or more surfactant protein, e.g., one or more of surfactant A protein, surfactant B protein, surfactant C protein, and surfactant D protein.

In certain embodiments the present invention provides a method for producing a therapeutic composition having full-length mRNA that encodes a peptide or protein for use in the delivery to or treatment of the liver of a subject or a liver cell. Such peptides and polypeptides can include those associated with a urea cycle disorder, associated with a lysosomal storage disorder, with a glycogen storage disorder, associated with an amino acid metabolism disorder, associated with a lipid metabolism or fibrotic disorder, associated with methylmalonic acidemia, or associated with any other metabolic disorder for which delivery to or treatment of the liver or a liver cell with enriched full-length mRNA provides therapeutic benefit.

In certain embodiments the present invention provides a method for producing a therapeutic composition having full-length mRNA that encodes for a protein associated with a urea cycle disorder. In certain embodiments the present invention provides a method for producing a therapeutic composition having full-length mRNA that encodes for ornithine transcarbamylase (OTC) protein. In certain embodiments the present invention provides a method for producing a therapeutic composition having full-length mRNA that encodes for arginosuccinate synthetase 1 protein. In certain embodiments the present invention provides a method for producing a therapeutic composition having full-length mRNA that encodes for carbamoyl phosphate synthetase I protein. In certain embodiments the present invention provides a method for producing a therapeutic composition having full-length mRNA that encodes for arginosuccinate lyase protein. In certain embodiments the present invention provides a method for producing a therapeutic composition having full-length mRNA that encodes for arginase protein.

In certain embodiments the present invention provides a method for producing a therapeutic composition having full-length mRNA that encodes for a protein associated with a lysosomal storage disorder. In certain embodiments the present invention provides a method for producing a therapeutic composition having full-length mRNA that encodes for alpha galactosidase protein. In certain embodiments the present invention provides a method for producing a therapeutic composition having full-length mRNA that encodes for glucocerebrosidase protein. In certain embodiments the present invention provides a method for producing a therapeutic composition having full-length mRNA that encodes for iduronate-2-sulfatase protein. In certain embodiments the present invention provides a method for producing a therapeutic composition having full-length mRNA that encodes for iduronidase protein. In certain embodiments the present invention provides a method for producing a therapeutic composition having full-length mRNA that encodes for N-acetyl-alpha-D-glucosaminidase protein. In certain embodiments the present invention provides a method for producing a therapeutic composition having full-length mRNA that encodes for heparan N-sulfatase protein. In certain embodiments the present invention provides a method for producing a therapeutic composition having full-length mRNA that encodes for galactosamine-6 sulfatase protein. In certain embodiments the present invention provides a method for producing a therapeutic composition having full-length mRNA that encodes for beta-galactosidase protein. In certain embodiments the present invention provides a method for producing a therapeutic composition having full-length mRNA that encodes for lysosomal lipase protein. In certain embodiments the present invention provides a method for producing a therapeutic composition having full-length mRNA that encodes for arylsulfatase B (N-acetylgalactosamine-4-sulfatase) protein. In certain embodiments the present invention provides a method for producing a therapeutic composition having full-length mRNA that encodes for transcription factor EB (TFEB).

In certain embodiments the present invention provides a method for producing a therapeutic composition having full-length mRNA that encodes for a protein associated with a glycogen storage disorder. In certain embodiments the present invention provides a method for producing a therapeutic composition having full-length mRNA that encodes for acid alpha-glucosidase protein. In certain embodiments the present invention provides a method for producing a therapeutic composition having full-length mRNA that encodes for glucose-6-phosphatase (G6PC) protein. In certain embodiments the present invention provides a method for producing a therapeutic composition having full-length mRNA that encodes for liver glycogen phosphorylase protein. In certain embodiments the present invention provides a method for producing a therapeutic composition having full-length mRNA that encodes for muscle phosphoglycerate mutase protein. In certain embodiments the present invention provides a method for producing a therapeutic composition having full-length mRNA that encodes for glycogen debranching enzyme.

In certain embodiments the present invention provides a method for producing a therapeutic composition having full-length mRNA that encodes for a protein associated with amino acid metabolism. In certain embodiments the present invention provides a method for producing a therapeutic composition having full-length mRNA that encodes for phenylalanine hydroxylase enzyme. In certain embodiments the present invention provides a method for producing a therapeutic composition having full-length mRNA that encodes for glutaryl-CoA dehydrogenase enzyme. In certain embodiments the present invention provides a method for producing a therapeutic composition having full-length mRNA that encodes for propionyl-CoA caboxylase enzyme. In certain embodiments the present invention provides a method for producing a therapeutic composition having full-length mRNA that encodes for oxalase alanine-glyoxylate aminotransferase enzyme.

In certain embodiments the present invention provides a method for producing a therapeutic composition having full-length mRNA that encodes for a protein associated with a lipid metabolism or fibrotic disorder. In certain embodiments the present invention provides a method for producing a therapeutic composition having full-length mRNA that encodes for a mTOR inhibitor. In certain embodiments the present invention provides a method for producing a therapeutic composition having full-length mRNA that encodes for ATPase phospholipid transporting 8B1 (ATP8B1) protein. In certain embodiments the present invention provides a method for producing a therapeutic composition having full-length mRNA that encodes for one or more NF-kappa B inhibitors, such as one or more of I-kappa B alpha, interferon-related development regulator 1 (IFRD1), and Sirtuin 1 (SIRT1). In certain embodiments the present invention provides a method for producing a therapeutic composition having full-length mRNA that encodes for PPAR-gamma protein or an active variant.

In certain embodiments the present invention provides a method for producing a therapeutic composition having full-length mRNA that encodes for a protein associated with methylmalonic acidemia. For example, in certain embodiments the present invention provides a method for producing a therapeutic composition having full-length mRNA that encodes for methylmalonyl CoA mutase protein. In certain embodiments the present invention provides a method for producing a therapeutic composition having full-length mRNA that encodes for methylmalonyl CoA epimerase protein.

In certain embodiments the present invention provides a method for producing a therapeutic composition having full-length mRNA for which delivery to or treatment of the liver can provide therapeutic benefit. In certain embodiments the present invention provides a method for producing a therapeutic composition having full-length mRNA that encodes for ATP7B protein, also known as Wilson disease protein. In certain embodiments the present invention provides a method for producing a therapeutic composition having full-length mRNA that encodes for porphobilinogen deaminase enzyme. In certain embodiments the present invention provides a method for producing a therapeutic composition having full-length mRNA that encodes for one or clotting enzymes, such as Factor VIII, Factor IX, Factor VII, and Factor X. In certain embodiments the present invention provides a method for producing a therapeutic composition having full-length mRNA that encodes for human hemochromatosis (HFE) protein.

In certain embodiments the present invention provides a method for producing a therapeutic composition having full-length mRNA that encodes a peptide or protein for use in the delivery to or treatment of the cardiovasculature of a subject or a cardiovascular cell. In certain embodiments the present invention provides a method for producing a therapeutic composition having full-length mRNA that encodes for vascular endothelial growth factor A protein. In certain embodiments the present invention provides a method for producing a therapeutic composition having full-length mRNA that encodes for relaxin protein. In certain embodiments the present invention provides a method for producing a therapeutic composition having full-length mRNA that encodes for bone morphogenetic protein-9 protein. In certain embodiments the present invention provides a method for producing a therapeutic composition having full-length mRNA that encodes for bone morphogenetic protein-2 receptor protein.

In certain embodiments the present invention provides a method for producing a therapeutic composition having full-length mRNA that encodes a peptide or protein for use in the delivery to or treatment of the muscle of a subject or a muscle cell. In certain embodiments the present invention provides a method for producing a therapeutic composition having full-length mRNA that encodes for dystrophin protein. In certain embodiments the present invention provides a method for producing a therapeutic composition having full-length mRNA that encodes for frataxin protein. In certain embodiments the present invention provides a method for producing a therapeutic composition having full-length mRNA that encodes a peptide or protein for use in the delivery to or treatment of the cardiac muscle of a subject or a cardiac muscle cell. In certain embodiments the present invention provides a method for producing a therapeutic composition having full-length mRNA that encodes for a protein that modulates one or both of a potassium channel and a sodium channel in muscle tissue or in a muscle cell. In certain embodiments the present invention provides a method for producing a therapeutic composition having full-length mRNA that encodes for a protein that modulates a Kv7.1 channel in muscle tissue or in a muscle cell. In certain embodiments the present invention provides a method for producing a therapeutic composition having full-length mRNA that encodes for a protein that modulates a Nav1.5 channel in muscle tissue or in a muscle cell.

In certain embodiments the present invention provides a method for producing a therapeutic composition having full-length mRNA that encodes a peptide or protein for use in the delivery to or treatment of the nervous system of a subject or a nervous system cell. For example, in certain embodiments the present invention provides a method for producing a therapeutic composition having full-length mRNA that encodes for survival motor neuron 1 protein. For example, in certain embodiments the present invention provides a method for producing a therapeutic composition having full-length mRNA that encodes for survival motor neuron 2 protein. In certain embodiments the present invention provides a method for producing a therapeutic composition having full-length mRNA that encodes for frataxin protein. In certain embodiments the present invention provides a method for producing a therapeutic composition having full-length mRNA that encodes for ATP binding cassette subfamily D member 1 (ABCD1) protein. In certain embodiments the present invention provides a method for producing a therapeutic composition having full-length mRNA that encodes for CLN3 protein.

In certain embodiments the present invention provides a method for producing a therapeutic composition having full-length mRNA that encodes a peptide or protein for use in the delivery to or treatment of the blood or bone marrow of a subject or a blood or bone marrow cell. In certain embodiments the present invention provides a method for producing a therapeutic composition having full-length mRNA that encodes for beta globin protein. In certain embodiments the present invention provides a method for producing a therapeutic composition having full-length mRNA that encodes for Bruton's tyrosine kinase protein. In certain embodiments the present invention provides a method for producing a therapeutic composition having full-length mRNA that encodes for one or clotting enzymes, such as Factor VIII, Factor IX, Factor VII, and Factor X.

In certain embodiments the present invention provides a method for producing a therapeutic composition having full-length mRNA that encodes a peptide or protein for use in the delivery to or treatment of the kidney of a subject or a kidney cell. In certain embodiments the present invention provides a method for producing a therapeutic composition having full-length mRNA that encodes for collagen type IV alpha 5 chain (COL4A5) protein.

In certain embodiments the present invention provides a method for producing a therapeutic composition having full-length mRNA that encodes a peptide or protein for use in the delivery to or treatment of the eye of a subject or an eye cell. In certain embodiments the present invention provides a method for producing a therapeutic composition having full-length mRNA that encodes for ATP-binding cassette sub-family A member 4 (ABCA4) protein. In certain embodiments the present invention provides a method for producing a therapeutic composition having full-length mRNA that encodes for retinoschisin protein. In certain embodiments the present invention provides a method for producing a therapeutic composition having full-length mRNA that encodes for retinal pigment epithelium-specific 65 kDa (RPE65) protein. In certain embodiments the present invention provides a method for producing a therapeutic composition having full-length mRNA that encodes for centrosomal protein of 290 kDa (CEP290).

In certain embodiments the present invention provides a method for producing a therapeutic composition having full-length mRNA that encodes a peptide or protein for use in the delivery of or treatment with a vaccine for a subject or a cell of a subject. For example, in certain embodiments the present invention provides a method for producing a therapeutic composition having full-length mRNA that encodes for an antigen from an infectious agent, such as a virus. In certain embodiments the present invention provides a method for producing a therapeutic composition having full-length mRNA that encodes for an antigen from influenza virus. In certain embodiments the present invention provides a method for producing a therapeutic composition having full-length mRNA that encodes for an antigen from respiratory syncytial virus. In certain embodiments the present invention provides a method for producing a therapeutic composition having full-length mRNA that encodes for an antigen from rabies virus. In certain embodiments the present invention provides a method for producing a therapeutic composition having full-length mRNA that encodes for an antigen from cytomegalovirus. In certain embodiments the present invention provides a method for producing a therapeutic composition having full-length mRNA that encodes for an antigen from rotavirus. In certain embodiments the present invention provides a method for producing a therapeutic composition having full-length mRNA that encodes for an antigen from a hepatitis virus, such as hepatitis A virus, hepatitis B virus, or hepatis C virus. In certain embodiments the present invention provides a method for producing a therapeutic composition having full-length mRNA that encodes for an antigen from human papillomavirus. In certain embodiments the present invention provides a method for producing a therapeutic composition having full-length mRNA that encodes for an antigen from a herpes simplex virus, such as herpes simplex virus 1 or herpes simplex virus 2. In certain embodiments the present invention provides a method for producing a therapeutic composition having full-length mRNA that encodes for an antigen from a human immunodeficiency virus, such as human immunodeficiency virus type 1 or human immunodeficiency virus type 2. In certain embodiments the present invention provides a method for producing a therapeutic composition having full-length mRNA that encodes for an antigen from a human metapneumovirus. In certain embodiments the present invention provides a method for producing a therapeutic composition having full-length mRNA that encodes for an antigen from a human parainfluenza virus, such as human parainfluenza virus type 1, human parainfluenza virus type 2, or human parainfluenza virus type 3. In certain embodiments the present invention provides a method for producing a therapeutic composition having full-length mRNA that encodes for an antigen from malaria virus. In certain embodiments the present invention provides a method for producing a therapeutic composition having full-length mRNA that encodes for an antigen from zika virus. In certain embodiments the present invention provides a method for producing a therapeutic composition having full-length mRNA that encodes for an antigen from chikungunya virus.

In certain embodiments the present invention provides a method for producing a therapeutic composition having full-length mRNA that encodes for an antigen associated with a cancer of a subject or identified from a cancer cell of a subject. In certain embodiments the present invention provides a method for producing a therapeutic composition having full-length mRNA that encodes for an antigen determined from a subject's own cancer cell, i.e., to provide a personalized cancer vaccine. In certain embodiments the present invention provides a method for producing a therapeutic composition having full-length mRNA that encodes for an antigen expressed from a mutant KRAS gene.

In certain embodiments the present invention provides a method for producing a therapeutic composition having full-length mRNA that encodes for an antibody. In certain embodiments, the antibody can be a bi-specific antibody. In certain embodiments, the antibody can be part of a fusion protein. In certain embodiments the present invention provides a method for producing a therapeutic composition having full-length mRNA that encodes for an antibody to OX40. In certain embodiments the present invention provides a method for producing a therapeutic composition having full-length mRNA that encodes for an antibody to VEGF. In certain embodiments the present invention provides a method for producing a therapeutic composition having full-length mRNA that encodes for an antibody to tissue necrosis factor alpha. In certain embodiments the present invention provides a method for producing a therapeutic composition having full-length mRNA that encodes for an antibody to CD3. In certain embodiments the present invention provides a method for producing a therapeutic composition having full-length mRNA that encodes for an antibody to CD19.

In certain embodiments the present invention provides a method for producing a therapeutic composition having full-length mRNA that encodes for an immunomodulator. In certain embodiments the present invention provides a method for producing a therapeutic composition having full-length mRNA that encodes for Interleukin 12. In certain embodiments the present invention provides a method for producing a therapeutic composition having full-length mRNA that encodes for Interleukin 23. In certain embodiments the present invention provides a method for producing a therapeutic composition having full-length mRNA that encodes for Interleukin 36 gamma. In certain embodiments the present invention provides a method for producing a therapeutic composition having full-length mRNA that encodes for a constitutively active variant of one or more stimulator of interferon genes (STING) proteins.

In certain embodiments the present invention provides a method for producing a therapeutic composition having full-length mRNA that encodes for an endonuclease. In certain embodiments the present invention provides a method for producing a therapeutic composition having full-length mRNA that encodes for an RNA-guided DNA endonuclease protein, such as Cas 9 protein. In certain embodiments the present invention provides a method for producing a therapeutic composition having full-length mRNA that encodes for a meganuclease protein. In certain embodiments the present invention provides a method for producing a therapeutic composition having full-length mRNA that encodes for a transcription activator-like effector nuclease protein. In certain embodiments the present invention provides a method for producing a therapeutic composition having full-length mRNA that encodes for a zinc finger nuclease protein.

In embodiments, exemplary therapeutic uses result from the delivery of mRNA encoding a secreted protein. Accordingly, in embodiments, the compositions and methods of the invention provide for delivery of mRNA encoding a secreted protein. In some embodiments, the compositions and methods of the invention provide for delivery of mRNA encoding one or more secreted proteins listed in Table 1; thus, compositions of the invention may comprise an mRNA encoding a protein listed in Table 1 (or a homolog thereof) along with other components set out herein, and methods of the invention may comprise preparing and/or administering a composition comprising an mRNA encoding a protein listed in Table 1 (or a homolog thereof) along with other components set out herein

TABLE 1
Secreted Proteins

| Uniprot ID | Protein Name | Gene Name |
|---|---|---|
| A1E959 | Odontogenic ameloblast-associated protein | ODAM |
| A1KZ92 | Peroxidasin-like protein | PXDNL |
| A1L453 | Serine protease 38 | PRSS38 |
| A1L4H1 | Soluble scavenger receptor cysteine-rich domain- | SSC5D |
| | containing protein SSC5D | |
| A2RUU4 | Colipase-like protein 1 | CLPSL1 |
| A2VDF0 | Fucose mutarotase | FUOM |
| A2VEC9 | SCO-spondin | SSPO |
| A3KMH1 | von Willebrand factor A domain-containing | VWA8 |
| | protein 8 | |
| A4D0S4 | Laminin subunit beta-4 | LAMB4 |
| A4D1T9 | Probable inactive serine protease 37 | PRSS37 |
| A5D8T8 | C-type lectin domain family 18 member A | CLEC18A |
| A6NC86 | phospholipase A2 inhibitor and Ly6/PLAUR | PINLYP |
| | domain-containing protein | |
| A6NCI4 | von Willebrand factor A domain-containing | VWA3A |
| | protein 3A | |
| A6ND01 | Probable folate receptor delta | FOLR4 |
| A6NDD2 | Beta-defensin 108B-like | |
| A6NE02 | BTB/POZ domain-containing protein 17 | BTBD17 |
| A6NEF6 | Growth hormone 1 | GH1 |
| A6NF02 | NPIP-like protein LOC730153 | |
| A6NFB4 | HCG1749481, isoform CRA_k | CSH1 |
| A6NFZ4 | Protein FAM24A | FAM24A |
| A6NG13 | Glycosyltransferase 54 domain-containing protein | |
| A6NGN9 | IgLON family member 5 | IGLON5 |
| A6NHN0 | Otolin-1 | OTOL1 |
| A6NHN6 | Nuclear pore complex-interacting protein-like 2 | NPIPL2 |
| A6NI73 | Leukocyte immunoglobulin-like receptor | LILRA5 |
| | subfamily A member 5 | |
| A6NIT4 | Chorionic somatomammotropin hormone 2 | CSH2 |
| | isoform 2 | |
| A6NJ69 | IgA-inducing protein homolog | IGIP |
| A6NKQ9 | Choriogonadotropin subunit beta variant 1 | CGB1 |
| A6NMZ7 | Collagen alpha-6(VI) chain | COL6A6 |
| A6NNS2 | Dehydrogenase/reductase SDR family member 7C | DHRS7C |
| A6XGL2 | Insulin A chain | INS |
| A8K0G1 | Protein Wnt | WNT7B |
| A8K2U0 | Alpha-2-macroglobulin-like protein 1 | A2ML1 |
| A8K7I4 | Calcium-activated chloride channel regulator 1 | CLCA1 |
| A8MTL9 | Serpin-like protein HMSD | HMSD |
| A8MV23 | Serpin E3 | SERPINE3 |
| A8MZH6 | Oocyte-secreted protein 1 homolog | OOSP1 |
| A8TX70 | Collagen alpha-5(VI) chain | COL6A5 |
| B0ZBE8 | Natriuretic peptide | NPPA |
| B1A4G9 | Somatotropin | GH1 |
| B1A4H2 | HCG1749481, isoform CRA_d | CSH1 |
| B1A4H9 | Chorionic somatomammotropin hormone | CSH2 |
| B1AJZ6 | Protein Wnt | WNT4 |
| B1AKI9 | Isthmin-1 | ISM1 |
| B2RNN3 | Complement C1q and tumor necrosis factor- | C1QTNF9B |
| | related protein 9B | |
| B2RUY7 | von Willebrand factor C domain-containing | VWC2L |
| | protein 2-like | |
| B3GLJ2 | Prostate and testis expressed protein 3 | PATE3 |
| B4DI03 | SEC11-like 3 (S. cerevisiae), isoform CRA_a | SEC11L3 |
| B4DJF9 | Protein Wnt | WNT4 |
| B4DUL4 | SEC11-like 1 (S. cerevisiae), isoform CRA_d | SEC11L1 |
| B5MCC8 | Protein Wnt | WNT10B |
| B8A595 | Protein Wnt | WNT7B |
| B8A597 | Protein Wnt | WNT7B |
| B8A598 | Protein Wnt | WNT7B |
| B9A064 | Immunoglobulin lambda-like polypeptide 5 | IGLL5 |
| C9J3H3 | Protein Wnt | WNT10B |
| C9J8I8 | Protein Wnt | WNT5A |
| C9JAF2 | Insulin-like growth factor II Ala-25 Del | IGF2 |
| C9JCI2 | Protein Wnt | WNT10B |
| C9JL84 | HERV-H LTR-associating protein 1 | HHLA1 |
| C9JNR5 | Insulin A chain | INS |
| C9JUI2 | Protein Wnt | WNT2 |
| D6RF47 | Protein Wnt | WNT8A |
| D6RF94 | Protein Wnt | WNT8A |
| E2RYF7 | Protein PBMUCL2 | HCG22 |
| E5RFR1 | PENK(114-133) | PENK |
| E7EML9 | Serine protease 44 | PRSS44 |
| E7EPC3 | Protein Wnt | WNT9B |
| E7EVP0 | Nociceptin | PNOC |
| E9PD02 | Insulin-like growth factor I | IGF1 |
| E9PH60 | Protein Wnt | WNT16 |
| E9PJL6 | Protein Wnt | WNT11 |
| F5GYM2 | Protein Wnt | WNT5B |
| F5H034 | Protein Wnt | WNT5B |
| F5H364 | Protein Wnt | WNT5B |
| F5H7Q6 | Protein Wnt | WNT5B |
| F8WCM5 | Protein INS-IGF2 | INS-IGF2 |
| F8WDR1 | Protein Wnt | WNT2 |
| H0Y663 | Protein Wnt | WNT4 |
| H0YK72 | Signal peptidase complex catalytic subunit | SEC11A |
| | SEC11A | |
| H0YK83 | Signal peptidase complex catalytic subunit | SEC11A |
| | SEC11A | |
| H0YM39 | Chorionic somatomammotropin hormone | CSH2 |
| H0YMT7 | Chorionic somatomammotropin hormone | CSH1 |
| H0YN17 | Chorionic somatomammotropin hormone | CSH2 |
| H0YNA5 | Signal peptidase complex catalytic subunit | SEC11A |
| | SEC11A | |
| H0YNG3 | Signal peptidase complex catalytic subunit | SEC11A |
| | SEC11A | |
| H0YNX5 | Signal peptidase complex catalytic subunit | SEC11A |
| | SEC11A | |
| H7BZB8 | Protein Wnt | WNT10A |
| H9KV56 | Choriogonadotropin subunit beta variant 2 | CGB2 |
| I3L0L8 | Protein Wnt | WNT9B |
| J3KNZ1 | Choriogonadotropin subunit beta variant 1 | CGB1 |
| J3KP00 | Choriogonadotropin subunit beta | CGB7 |
| J3QT02 | Choriogonadotropin subunit beta variant 1 | CGB1 |
| O00175 | C-C motif chemokine 24 | CCL24 |
| O00182 | Galectin-9 | LGALS9 |
| O00187 | Mannan-binding lectin serine protease 2 | MASP2 |
| O00230 | Cortistatin | CORT |
| O00253 | Agouti-related protein | AGRP |
| O00270 | 12-(S)-hydroxy-5,8,10,14-eicosatetraenoic acid | GPR31 |
| | receptor | |
| O00292 | Left-right determination factor 2 | LEFTY2 |
| O00294 | Tubby-related protein 1 | TULP1 |
| O00295 | Tubby-related protein 2 | TULP2 |
| O00300 | Tumor necrosis factor receptor superfamily | TNFRSF11B |
| | member 11B | |
| O00339 | Matrilin-2 | MATN2 |
| O00391 | Sulfhydryl oxidase 1 | QSOX1 |
| O00468 | Agrin | AGRN |
| O00515 | Ladinin-1 | LAD1 |
| O00533 | Processed neural cell adhesion molecule L1-like | CHL1 |
| | protein | |
| O00584 | Ribonuclease T2 | RNASET2 |
| O00585 | C-C motif chemokine 21 | CCL21 |
| O00602 | Ficolin-1 | FCN1 |
| O00622 | Protein CYR61 | CYR61 |
| O00626 | MDC(5-69) | CCL22 |
| O00634 | Netrin-3 | NTN3 |
| O00744 | Protein Wnt-10b | WNT10B |
| O00755 | Protein Wnt-7a | WNT7A |
| O14498 | Immunoglobulin superfamily containing leucine- | ISLR |
| | rich repeat protein | |
| O14511 | Pro-neuregulin-2, membrane-bound isoform | NRG2 |
| O14594 | Neurocan core protein | NCAN |
| O14625 | C-X-C motif chemokine 11 | CXCL11 |
| O14638 | Ectonucleotide pyrophosphatase/phosphodiesterase | ENPP3 |
| | family member 3 | |
| O14656 | Torsin-1A | TOR1A |
| O14657 | Torsin-1B | TOR1B |
| O14786 | Neuropilin-1 | NRP1 |
| O14788 | Tumor necrosis factor ligand superfamily member | TNFSF11 |
| | 11, membrane form | |
| O14791 | Apolipoprotein L1 | APOL1 |
| O14793 | Growth/differentiation factor 8 | MSTN |
| O14904 | Protein Wnt-9a | WNT9A |
| O14905 | Protein Wnt-9b | WNT9B |
| O14944 | Proepiregulin | EREG |
| O14960 | Leukocyte cell-derived chemotaxin-2 | LECT2 |
| O15018 | Processed PDZ domain-containing protein 2 | PDZD2 |
| O15041 | Semaphorin-3E | SEMA3E |
| O15072 | A disintegrin and metalloproteinase with | ADAMTS3 |
| | thrombospondin motifs 3 | |
| O15123 | Angiopoietin-2 | ANGPT2 |
| O15130 | Neuropeptide FF | NPFF |
| O15197 | Ephrin type-B receptor 6 | EPHB6 |
| O15204 | ADAM DEC1 | ADAMDEC1 |
| O15230 | Laminin subunit alpha-5 | LAMA5 |
| O15232 | Matrilin-3 | MATN3 |
| O15240 | Neuroendocrine regulatory peptide-1 | VGF |
| O15263 | Beta-defensin 4A | DEFB4A |
| O15335 | Chondroadherin | CHAD |
| O15393 | Transmembrane protease serine 2 catalytic chain | TMPRSS2 |
| O15444 | C-C motif chemokine 25 | CCL25 |
| O15467 | C-C motif chemokine 16 | CCL16 |
| O15496 | Group 10 secretory phospholipase A2 | PLA2G10 |
| O15520 | Fibroblast growth factor 10 | FGF10 |
| O15537 | Retinoschisin | RS1 |
| O43157 | Plexin-B1 | PLXNB1 |
| O43184 | Disintegrin and metalloproteinase domain- | ADAM12 |
| | containing protein 12 | |
| O43240 | Kallikrein-10 | KLK10 |
| O43278 | Kunitz-type protease inhibitor 1 | SPINT1 |
| O43320 | Fibroblast growth factor 16 | FGF16 |
| O43323 | Desert hedgehog protein C-product | DHH |
| O43405 | Cochlin | COCH |
| O43508 | Tumor necrosis factor ligand superfamily member | TNFSF12 |
| | 12, membrane form | |
| O43555 | Progonadoliberin-2 | GNRH2 |
| O43557 | Tumor necrosis factor ligand superfamily member | TNFSF14 |
| | 14, soluble form | |
| O43692 | Peptidase inhibitor 15 | PI15 |
| O43699 | Sialic acid-binding Ig-like lectin 6 | SIGLEC6 |
| O43820 | Hyaluronidase-3 | HYAL3 |
| O43827 | Angiopoietin-related protein 7 | ANGPTL7 |
| O43852 | Calumenin | CALU |
| O43854 | EGF-like repeat and discoidin I-like domain- | EDIL3 |
| | containing protein 3 | |
| O43866 | CD5 antigen-like | CD5L |
| O43897 | Tolloid-like protein 1 | TLL1 |
| O43915 | Vascular endothelial growth factor D | FIGF |
| O43927 | C-X-C motif chemokine 13 | CXCL13 |
| O60218 | Aldo-keto reductase family 1 member B10 | AKR1B10 |
| O60235 | Transmembrane protease serine 11D | TMPRSS11D |
| O60258 | Fibroblast growth factor 17 | FGF17 |
| O60259 | Kallikrein-8 | KLK8 |
| O60383 | Growth/differentiation factor 9 | GDF9 |
| O60469 | Down syndrome cell adhesion molecule | DSCAM |
| O60542 | Persephin | PSPN |
| O60565 | Gremlin-1 | GREM1 |
| O60575 | Serine protease inhibitor Kazal-type 4 | SPINK4 |
| O60676 | Cystatin-8 | CST8 |
| O60687 | Sushi repeat-containing protein SRPX2 | SRPX2 |
| O60844 | Zymogen granule membrane protein 16 | ZG16 |
| O60882 | Matrix metalloproteinase-20 | MMP20 |
| O60938 | Keratocan | KERA |
| O75015 | Low affinity immunoglobulin gamma Fc region | FCGR3B |
| | receptor III-B | |
| O75077 | Disintegrin and metalloproteinase domain- | ADAM23 |
| | containing protein 23 | |
| O75093 | Slit homolog 1 protein | SLIT1 |
| O75094 | Slit homolog 3 protein | SLIT3 |
| O75095 | Multiple epidermal growth factor-like domains | MEGF6 |
| | protein 6 | |
| O75173 | A disintegrin and metalloproteinase with | ADAMTS4 |
| | thrombospondin motifs 4 | |
| O75200 | Nuclear pore complex-interacting protein-like 1 | NPIPL1 |
| O75339 | Cartilage intermediate layer protein 1 C1 | CILP |
| O75354 | Ectonucleoside triphosphate diphosphohydrolase 6 | ENTPD6 |
| O75386 | Tubby-related protein 3 | TULP3 |
| O75398 | Deformed epidermal autoregulatory factor 1 | DEAF1 |
| | homolog | |
| O75443 | Alpha-tectorin | TECTA |
| O75445 | Usherin | USH2A |
| O75462 | Cytokine receptor-like factor 1 | CRLF1 |
| O75487 | Glypican-4 | GPC4 |
| O75493 | Carbonic anhydrase-related protein 11 | CA11 |
| O75594 | Peptidoglycan recognition protein 1 | PGLYRP1 |
| O75596 | C-type lectin domain family 3 member A | CLEC3A |
| O75610 | Left-right determination factor 1 | LEFTY1 |
| O75629 | Protein CREG1 | CREG1 |
| O75636 | Ficolin-3 | FCN3 |
| O75711 | Scrapie-responsive protein 1 | SCRG1 |
| O75715 | Epididymal secretory glutathione peroxidase | GPX5 |
| O75718 | Cartilage-associated protein | CRTAP |
| O75829 | Chondrosurfactant protein | LECT1 |
| O75830 | Serpin I2 | SERPINI2 |
| O75882 | Attractin | ATRN |
| O75888 | Tumor necrosis factor ligand superfamily | TNFSF13 |
| | member 13 | |
| O75900 | Matrix metalloproteinase-23 | MMP23A |
| O75951 | Lysozyme-like protein 6 | LYZL6 |
| O75973 | C1q-related factor | C1QL1 |
| O76038 | Secretagogin | SCGN |
| O76061 | Stanniocalcin-2 | STC2 |
| O76076 | WNT1-inducible-signaling pathway protein 2 | WISP2 |
| O76093 | Fibroblast growth factor 18 | FGF18 |
| O76096 | Cystatin-F | CST7 |
| O94769 | Extracellular matrix protein 2 | ECM2 |
| O94813 | Slit homolog 2 protein C-product | SLIT2 |
| O94907 | Dickkopf-related protein 1 | DKK1 |
| O94919 | Endonuclease domain-containing 1 protein | ENDOD1 |
| O94964 | N-terminal form | SOGA1 |
| O95025 | Semaphorin-3D | SEMA3D |
| O95084 | Serine protease 23 | PRSS23 |
| O95150 | Tumor necrosis factor ligand superfamily | TNFSF15 |
| | member 15 | |
| O95156 | Neurexophilin-2 | NXPH2 |
| O95157 | Neurexophilin-3 | NXPH3 |
| O95158 | Neurexophilin-4 | NXPH4 |
| O95388 | WNT1-inducible-signaling pathway protein 1 | WISP1 |
| O95389 | WNT1-inducible-signaling pathway protein 3 | WISP3 |
| O95390 | Growth/differentiation factor 11 | GDF11 |
| O95393 | Bone morphogenetic protein 10 | BMP10 |
| O95399 | Urotensin-2 | UTS2 |
| O95407 | Tumor necrosis factor receptor superfamily | TNFRSF6B |
| | member 6B | |
| O95428 | Papilin | PAPLN |
| O95445 | Apolipoprotein M | APOM |
| O95450 | A disintegrin and metalloproteinase with | ADAMTS2 |
| | thrombospondin motifs 2 | |
| O95460 | Matrilin-4 | MATN4 |
| O95467 | LHAL tetrapeptide | GNAS |
| O95631 | Netrin-1 | NTN1 |
| O95633 | Follistatin-related protein 3 | FSTL3 |
| O95711 | Lymphocyte antigen 86 | LY86 |
| O95715 | C-X-C motif chemokine 14 | CXCL14 |
| O95750 | Fibroblast growth factor 19 | FGF19 |
| O95760 | Interleukin-33 | IL33 |
| O95813 | Cerberus | CER1 |
| O95841 | Angiopoietin-related protein 1 | ANGPTL1 |
| O95897 | Noelin-2 | OLFM2 |
| O95925 | Eppin | EPPIN |
| O95965 | Integrin beta-like protein 1 | ITGBL1 |
| O95967 | EGF-containing fibulin-like extracellular matrix | EFEMP2 |
| | protein 2 | |
| O95968 | Secretoglobin family 1D member 1 | SCGB1D1 |
| O95969 | Secretoglobin family 1D member 2 | SCGB1D2 |
| O95970 | Leucine-rich glioma-inactivated protein 1 | LGI1 |
| O95972 | Bone morphogenetic protein 15 | BMP15 |
| O95994 | Anterior gradient protein 2 homolog | AGR2 |
| O95998 | Interleukin-18-binding protein | IL18BP |
| O96009 | Napsin-A | NAPSA |
| O96014 | Protein Wnt-11 | WNT11 |
| P00450 | Ceruloplasmin | CP |
| P00451 | Factor VIIIa light chain | F8 |
| P00488 | Coagulation factor XIII A chain | F13A1 |
| P00533 | Epidermal growth factor receptor | EGFR |
| P00709 | Alpha-lactalbumin | LALBA |
| P00734 | Prothrombin | F2 |
| P00738 | Haptoglobin beta chain | HP |
| P00739 | Haptoglobin-related protein | HPR |
| P00740 | Coagulation factor IXa heavy chain | F9 |
| P00742 | Factor X heavy chain | F10 |
| P00746 | Complement factor D | CFD |
| P00747 | Plasmin light chain B | PLG |
| P00748 | Coagulation factor XIIa light chain | F12 |
| P00749 | Urokinase-type plasminogen activator long | PLAU |
| | chain A | |
| P00750 | Tissue-type plasminogen activator | PLAT |
| P00751 | Complement factor B Ba fragment | CFB |
| P00797 | Renin | REN |
| P00973 | 2′-5′-oligoadenylate synthase 1 | OAS1 |
| P00995 | Pancreatic secretory trypsin inhibitor | SPINK1 |
| P01008 | Antithrombin-III | SERPINC1 |
| P01009 | Alpha-1-antitrypsin | SERPINA1 |
| P01011 | Alpha-1-antichymotrypsin His-Pro-less | SERPINA3 |
| P01019 | Angiotensin-1 | AGT |
| P01023 | Alpha-2-macroglobulin | A2M |
| P01024 | Acylation stimulating protein | C3 |
| P01031 | Complement C5 beta chain | C5 |
| P01033 | Metalloproteinase inhibitor 1 | TIMP1 |
| P01034 | Cystatin-C | CST3 |
| P01036 | Cystatin-S | CST4 |
| P01037 | Cystatin-SN | CST1 |
| P01042 | Kininogen-1 light chain | KNG1 |
| P01127 | Platelet-derived growth factor subunit B | PDGFB |
| P01135 | Transforming growth factor alpha | TGFA |
| P01137 | Transforming growth factor beta-1 | TGFB1 |
| P01138 | Beta-nerve growth factor | NGF |
| P01148 | Gonadoliberin-1 | GNRH1 |
| P01160 | Atrial natriuretic factor | NPPA |
| P01178 | Oxytocin | OXT |
| P01185 | Vasopressin-neurophysin 2-copeptin | AVP |
| P01189 | Corticotropin | POMC |
| P01210 | PENK(237-258) | PENK |
| P01213 | Alpha-neoendorphin | PDYN |
| P01215 | Glycoprotein hormones alpha chain | CGA |
| P01222 | Thyrotropin subunit beta | TSHB |
| P01225 | Follitropin subunit beta | FSHB |
| P01229 | Lutropin subunit beta | LHB |
| P01233 | Choriogonadotropin subunit beta | CGB8 |
| P01236 | Prolactin | PRL |
| P01241 | Somatotropin | GH1 |
| P01242 | Growth hormone variant | GH2 |
| P01243 | Chorionic somatomammotropin hormone | CSH2 |
| P01258 | Katacalcin | CALCA |
| P01266 | Thyroglobulin | TG |
| P01270 | Parathyroid hormone | PTH |
| P01275 | Glucagon | GCG |
| P01282 | Intestinal peptide PHM-27 | VIP |
| P01286 | Somatoliberin | GHRH |
| P01298 | Pancreatic prohormone | PPY |
| P01303 | C-flanking peptide of NPY | NPY |
| P01308 | Insulin | INS |
| P01344 | Insulin-like growth factor II | IGF2 |
| P01350 | Big gastrin | GAST |
| P01374 | Lymphotoxin-alpha | LTA |
| P01375 | C-domain 1 | TNF |
| P01562 | Interferon alpha-1/13 | IFNA1 |
| P01563 | Interferon alpha-2 | IFNA2 |
| P01566 | Interferon alpha-10 | IFNA10 |
| P01567 | Interferon alpha-7 | IFNA7 |
| P01568 | Interferon alpha-21 | IFNA21 |
| P01569 | Interferon alpha-5 | IFNA5 |
| P01570 | Interferon alpha-14 | IFNA14 |
| P01571 | Interferon alpha-17 | IFNA17 |
| P01574 | Interferon beta | IFNB1 |
| P01579 | Interferon gamma | IFNG |
| P01583 | Interleukin-1 alpha | IL1A |
| P01584 | Interleukin-1 beta | IL1B |
| P01588 | Erythropoietin | EPO |
| P01591 | Immunoglobulin J chain | IGJ |
| P01732 | T-cell surface glycoprotein CD8 alpha chain | CD8A |
| P01833 | Polymeric immunoglobulin receptor | PIGR |
| P01857 | Ig gamma-1 chain C region | IGHG1 |
| P01859 | Ig gamma-2 chain C region | IGHG2 |
| P01860 | Ig gamma-3 chain C region | IGHG3 |
| P01861 | Ig gamma-4 chain C region | IGHG4 |
| P01871 | Ig mu chain C region | IGHM |
| P01880 | Ig delta chain C region | IGHD |
| P02452 | Collagen alpha-1(I) chain | COL1A1 |
| P02458 | Chondrocalcin | COL2A1 |
| P02461 | Collagen alpha-1(III) chain | COL3A1 |
| P02462 | Collagen alpha-1(IV) chain | COL4A1 |
| P02647 | Apolipoprotein A-I | APOA1 |
| P02649 | Apolipoprotein E | APOE |
| P02652 | Apolipoprotein A-II | APOA2 |
| P02654 | Apolipoprotein C-I | APOC1 |
| P02655 | Apolipoprotein C-II | APOC2 |
| P02656 | Apolipoprotein C-III | APOC3 |
| P02671 | Fibrinogen alpha chain | FGA |
| P02675 | Fibrinopeptide B | FGB |
| P02679 | Fibrinogen gamma chain | FGG |
| P02741 | C-reactive protein | CRP |
| P02743 | Serum amyloid P-component(1-203) | APCS |
| P02745 | Complement C1q subcomponent subunit A | C1QA |
| P02746 | Complement C1q subcomponent subunit B | C1QB |
| P02747 | Complement C1q subcomponent subunit C | C1QC |
| P02748 | Complement component C9b | C9 |
| P02749 | Beta-2-glycoprotein 1 | APOH |
| P02750 | Leucine-rich alpha-2-glycoprotein | LRG1 |
| P02751 | Ugl-Y2 | FN1 |
| P02753 | Retinol-binding protein 4 | RBP4 |
| P02760 | Trypstatin | AMBP |
| P02763 | Alpha-1-acid glycoprotein 1 | ORM1 |
| P02765 | Alpha-2-HS-glycoprotein chain A | AHSG |
| P02766 | Transthyretin | TTR |
| P02768 | Serum albumin | ALB |
| P02771 | Alpha-fetoprotein | AFP |
| P02774 | Vitamin D-binding protein | GC |
| P02775 | Connective tissue-activating peptide III | PPBP |
| P02776 | Platelet factor 4 | PF4 |
| P02778 | CXCL10(1-73) | CXCL10 |
| P02786 | Transferrin receptor protein 1 | TFRC |
| P02787 | Serotransferrin | TF |
| P02788 | Lactoferroxin-C | LTF |
| P02790 | Hemopexin | HPX |
| P02808 | Statherin | STATH |
| P02810 | Salivary acidic proline-rich phosphoprotein 1/2 | PRH2 |
| P02812 | Basic salivary proline-rich protein 2 | PRB2 |
| P02814 | Peptide D1A | SMR3B |
| P02818 | Osteocalcin | BGLAP |
| P03950 | Angiogenin | ANG |
| P03951 | Coagulation factor XIa heavy chain | F11 |
| P03952 | Plasma kallikrein | KLKB1 |
| P03956 | 27 kDa interstitial collagenase | MMP1 |
| P03971 | Muellerian-inhibiting factor | AMH |
| P03973 | Antileukoproteinase | SLPI |
| P04003 | C4b-binding protein alpha chain | C4BPA |
| P04004 | Somatomedin-B | VTN |
| P04054 | Phospholipase A2 | PLA2G1B |
| P04085 | Platelet-derived growth factor subunit A | PDGFA |
| P04090 | Relaxin A chain | RLN2 |
| P04114 | Apolipoprotein B-100 | APOB |
| P04118 | Colipase | CLPS |
| P04141 | Granulocyte-macrophage colony-stimulating | CSF2 |
| | factor | |
| P04155 | Trefoil factor 1 | TFF1 |
| P04180 | Phosphatidylcholine-sterol acyltransferase | LCAT |
| P04196 | Histidine-rich glycoprotein | HRG |
| P04217 | Alpha-1B-glycoprotein | A1BG |
| P04275 | von Willebrand antigen 2 | VWF |
| P04278 | Sex hormone-binding globulin | SHBG |
| P04279 | Alpha-inhibin-31 | SEMG1 |
| P04280 | Basic salivary proline-rich protein 1 | PRB1 |
| P04628 | Proto-oncogene Wnt-1 | WNT1 |
| P04745 | Alpha-amylase 1 | AMY1A |
| P04746 | Pancreatic alpha-amylase | AMY2A |
| P04808 | Prorelaxin H1 | RLN1 |
| P05000 | Interferon omega-1 | IFNW1 |
| P05013 | Interferon alpha-6 | IFNA6 |
| P05014 | Interferon alpha-4 | IFNA4 |
| P05015 | Interferon alpha-16 | IFNA16 |
| P05019 | Insulin-like growth factor I | IGF1 |
| P05060 | GAWK peptide | CHGB |
| P05090 | Apolipoprotein D | APOD |
| P05109 | Protein S100-A8 | S100A8 |
| P05111 | Inhibin alpha chain | INHA |
| P05112 | Interleukin-4 | IL4 |
| P05113 | Interleukin-5 | IL5 |
| P05120 | Plasminogen activator inhibitor 2 | SERPINB2 |
| P05121 | Plasminogen activator inhibitor 1 | SERPINE1 |
| P05154 | Plasma serine protease inhibitor | SERPINA5 |
| P05155 | Plasma protease C1 inhibitor | SERPING1 |
| P05156 | Complement factor I heavy chain | CFI |
| P05160 | Coagulation factor XIII B chain | F13B |
| P05161 | Ubiquitin-like protein ISG15 | ISG15 |
| P05230 | Fibroblast growth factor 1 | FGF1 |
| P05231 | Interleukin-6 | IL6 |
| P05305 | Big endothelin-1 | EDN1 |
| P05408 | C-terminal peptide | SCG5 |
| P05451 | Lithostathine-1-alpha | REG1A |
| P05452 | Tetranectin | CLEC3B |
| P05543 | Thyroxine-binding globulin | SERPINA7 |
| P05814 | Beta-casein | CSN2 |
| P05997 | Collagen alpha-2(V) chain | COL5A2 |
| P06276 | Cholinesterase | BCHE |
| P06307 | Cholecystokinin-12 | CCK |
| P06396 | Gelsolin | GSN |
| P06681 | Complement C2 | C2 |
| P06702 | Protein S100-A9 | S100A9 |
| P06727 | Apolipoprotein A-IV | APOA4 |
| P06734 | Low affinity immunoglobulin epsilon Fc receptor | FCER2 |
| | soluble form | |
| P06744 | Glucose-6-phosphate isomerase | GPI |
| P06850 | Corticoliberin | CRH |
| P06858 | Lipoprotein lipase | LPL |
| P06881 | Calcitonin gene-related peptide 1 | CALCA |
| P07093 | Glia-derived nexin | SERPINE2 |
| P07098 | Gastric triacylglycerol lipase | LIPF |
| P07225 | Vitamin K-dependent protein S | PROS1 |
| P07237 | Protein disulfide-isomerase | P4HB |
| P07288 | Prostate-specific antigen | KLK3 |
| P07306 | Asialoglycoprotein receptor 1 | ASGR1 |
| P07355 | Annexin A2 | ANXA2 |
| P07357 | Complement component C8 alpha chain | C8A |
| P07358 | Complement component C8 beta chain | C8B |
| P07360 | Complement component C8 gamma chain | C8G |
| P07477 | Alpha-trypsin chain 2 | PRSS1 |
| P07478 | Trypsin-2 | PRSS2 |
| P07492 | Neuromedin-C | GRP |
| P07498 | Kappa-casein | CSN3 |
| P07585 | Decorin | DCN |
| P07911 | Uromodulin | UMOD |
| P07942 | Laminin subunit beta-1 | LAMB1 |
| P07988 | Pulmonary surfactant-associated protein B | SFTPB |
| P07998 | Ribonuclease pancreatic | RNASE1 |
| P08118 | Beta-microseminoprotein | MSMB |
| P08123 | Collagen alpha-2(I) chain | COL1A2 |
| P08185 | Corticosteroid-binding globulin | SERPINA6 |
| P08217 | Chymotrypsin-like elastase family member 2A | CELA2A |
| P08218 | Chymotrypsin-like elastase family member 2B | CELA2B |
| P08253 | 72 kDa type IV collagenase | MMP2 |
| P08254 | Stromelysin-1 | MMP3 |
| P08294 | Extracellular superoxide dismutase [Cu—Zn] | SOD3 |
| P08476 | Inhibin beta A chain | INHBA |
| P08493 | Matrix Gla protein | MGP |
| P08572 | Collagen alpha-2(IV) chain | COL4A2 |
| P08581 | Hepatocyte growth factor receptor | MET |
| P08603 | Complement factor H | CFH |
| P08620 | Fibroblast growth factor 4 | FGF4 |
| P08637 | Low affinity immunoglobulin gamma Fc region | FCGR3A |
| | receptor III-A | |
| P08697 | Alpha-2-antiplasmin | SERPINF2 |
| P08700 | Interleukin-3 | IL3 |
| P08709 | Coagulation factor VII | F7 |
| P08833 | Insulin-like growth factor-binding protein 1 | IGFBP1 |
| P08887 | Interleukin-6 receptor subunit alpha | IL6R |
| P08949 | Neuromedin-B-32 | NMB |
| P08F94 | Fibrocystin | PKHD1 |
| P09038 | Fibroblast growth factor 2 | FGF2 |
| P09228 | Cystatin-SA | CST2 |
| P09237 | Matrilysin | MMP7 |
| P09238 | Stromelysin-2 | MMP10 |
| P09341 | Growth-regulated alpha protein | CXCL1 |
| P09382 | Galectin-1 | LGALS1 |
| P09466 | Glycodelin | PAEP |
| P09486 | SPARC | SPARC |
| P09529 | Inhibin beta B chain | INHBB |
| P09544 | Protein Wnt-2 | WNT2 |
| P09603 | Processed macrophage colony-stimulating factor 1 | CSF1 |
| P09681 | Gastric inhibitory polypeptide | GIP |
| P09683 | Secretin | SCT |
| P09919 | Granulocyte colony-stimulating factor | CSF3 |
| P0C091 | FRAS1-related extracellular matrix protein 3 | FREM3 |
| P0C0L4 | C4d-A | C4A |
| P0C0L5 | Complement C4-B alpha chain | C4B |
| P0C0P6 | Neuropeptide S | NPS |
| P0C7L1 | Serine protease inhibitor Kazal-type 8 | SPINK8 |
| P0C862 | Complement C1q and tumor necrosis factor- | C1QTNF9 |
| | related protein 9A | |
| P0C8F1 | Prostate and testis expressed protein 4 | PATE4 |
| P0CG01 | Gastrokine-3 | GKN3P |
| P0CG36 | Cryptic family protein 1B | CFC1B |
| P0CG37 | Cryptic protein | CFC1 |
| P0CJ68 | Humanin-like protein 1 | MTRNR2L1 |
| P0CJ69 | Humanin-like protein 2 | MTRNR2L2 |
| P0CJ70 | Humanin-like protein 3 | MTRNR2L3 |
| P0CJ71 | Humanin-like protein 4 | MTRNR2L4 |
| P0CJ72 | Humanin-like protein 5 | MTRNR2L5 |
| P0CJ73 | Humanin-like protein 6 | MTRNR2L6 |
| P0CJ74 | Humanin-like protein 7 | MTRNR2L7 |
| P0CJ75 | Humanin-like protein 8 | MTRNR2L8 |
| P0CJ76 | Humanin-like protein 9 | MTRNR2L9 |
| P0CJ77 | Humanin-like protein 10 | MTRNR2L10 |
| P0DJD7 | Pepsin A-4 | PGA4 |
| P0DJD8 | Pepsin A-3 | PGA3 |
| P0DJD9 | Pepsin A-5 | PGA5 |
| P0DJI8 | Amyloid protein A | SAA1 |
| P0DJI9 | Serum amyloid A-2 protein | SAA2 |
| P10082 | Peptide YY(3-36) | PYY |
| P10092 | Calcitonin gene-related peptide 2 | CALCB |
| P10124 | Serglycin | SRGN |
| P10145 | MDNCF-a | IL8 |
| P10147 | MIP-1-alpha(4-69) | CCL3 |
| P10163 | Peptide P-D | PRB4 |
| P10451 | Osteopontin | SPP1 |
| P10599 | Thioredoxin | TXN |
| P10600 | Transforming growth factor beta-3 | TGFB3 |
| P10643 | Complement component C7 | C7 |
| P10645 | Vasostatin-2 | CHGA |
| P10646 | Tissue factor pathway inhibitor | TFPI |
| P10720 | Platelet factor 4 variant(4-74) | PF4V1 |
| P10745 | Retinol-binding protein 3 | RBP3 |
| P10767 | Fibroblast growth factor 6 | FGF6 |
| P10909 | Clusterin alpha chain | CLU |
| P10912 | Growth hormone receptor | GHR |
| P10915 | Hyaluronan and proteoglycan link protein 1 | HAPLN1 |
| P10966 | T-cell surface glycoprotein CD8 beta chain | CD8B |
| P10997 | Islet amyloid polypeptide | IAPP |
| P11047 | Laminin subunit gamma-1 | LAMC1 |
| P11150 | Hepatic triacylglycerol lipase | LIPC |
| P11226 | Mannose-binding protein C | MBL2 |
| P11464 | Pregnancy-specific beta-1-glycoprotein 1 | PSG1 |
| P11465 | Pregnancy-specific beta-1-glycoprotein 2 | PSG2 |
| P11487 | Fibroblast growth factor 3 | FGF3 |
| P11597 | Cholesteryl ester transfer protein | CETP |
| P11684 | Uteroglobin | SCGB1A1 |
| P11686 | Pulmonary surfactant-associated protein C | SFTPC |
| P12034 | Fibroblast growth factor 5 | FGF5 |
| P12107 | Collagen alpha-1(XI) chain | COL11A1 |
| P12109 | Collagen alpha-1(VI) chain | COL6A1 |
| P12110 | Collagen alpha-2(VI) chain | COL6A2 |
| P12111 | Collagen alpha-3(VI) chain | COL6A3 |
| P12259 | Coagulation factor V | F5 |
| P12272 | PTHrP[1-36] | PTHLH |
| P12273 | Prolactin-inducible protein | PIP |
| P12544 | Granzyme A | GZMA |
| P12643 | Bone morphogenetic protein 2 | BMP2 |
| P12644 | Bone morphogenetic protein 4 | BMP4 |
| P12645 | Bone morphogenetic protein 3 | BMP3 |
| P12724 | Eosinophil cationic protein | RNASE3 |
| P12821 | Angiotensin-converting enzyme, soluble form | ACE |
| P12838 | Neutrophil defensin 4 | DEFA4 |
| P12872 | Motilin | MLN |
| P13232 | Interleukin-7 | IL7 |
| P13236 | C-C motif chemokine 4 | CCL4 |
| P13284 | Gamma-interferon-inducible lysosomal thiol | IFI30 |
| | reductase | |
| P13500 | C-C motif chemokine 2 | CCL2 |
| P13501 | C-C motif chemokine 5 | CCL5 |
| P13521 | Secretogranin-2 | SCG2 |
| P13591 | Neural cell adhesion molecule 1 | NCAM1 |
| P13611 | Versican core protein | VCAN |
| P13671 | Complement component C6 | C6 |
| P13688 | Carcinoembryonic antigen-related cell adhesion | CEACAM1 |
| | molecule 1 | |
| P13725 | Oncostatin-M | OSM |
| P13726 | Tissue factor | F3 |
| P13727 | Eosinophil granule major basic protein | PRG2 |
| P13942 | Collagen alpha-2(XI) chain | COL11A2 |
| P13987 | CD59 glycoprotein | CD59 |
| P14138 | Endothelin-3 | EDN3 |
| P14174 | Macrophage migration inhibitory factor | MIF |
| P14207 | Folate receptor beta | FOLR2 |
| P14222 | Perforin-1 | PRF1 |
| P14543 | Nidogen-1 | NID1 |
| P14555 | Phospholipase A2, membrane associated | PLA2G2A |
| P14625 | Endoplasmin | HSP90B1 |
| P14735 | Insulin-degrading enzyme | IDE |
| P14778 | Interleukin-1 receptor type 1, soluble form | IL1R1 |
| P14780 | 82 kDa matrix metalloproteinase-9 | MMP9 |
| P15018 | Leukemia inhibitory factor | LIF |
| P15085 | Carboxypeptidase A1 | CPA1 |
| P15086 | Carboxypeptidase B | CPB1 |
| P15151 | Poliovirus receptor | PVR |
| P15169 | Carboxypeptidase N catalytic chain | CPN1 |
| P15248 | Interleukin-9 | IL9 |
| P15291 | N-acetyllactosamine synthase | B4GALT1 |
| P15309 | PAPf39 | ACPP |
| P15328 | Folate receptor alpha | FOLR1 |
| P15374 | Ubiquitin carboxyl-terminal hydrolase isozyme L3 | UCHL3 |
| P15502 | Elastin | ELN |
| P15509 | Granulocyte-macrophage colony-stimulating | CSF2RA |
| | factor receptor subunit alpha | |
| P15515 | Histatin-1 | HTN1 |
| P15516 | His3-(31-51)-peptide | HTN3 |
| P15692 | Vascular endothelial growth factor A | VEGFA |
| P15814 | Immunoglobulin lambda-like polypeptide 1 | IGLL1 |
| P15907 | Beta-galactoside alpha-2,6-sialyltransferase 1 | ST6GAL1 |
| P15941 | Mucin-1 subunit beta | MUC1 |
| P16035 | Metalloproteinase inhibitor 2 | TIMP2 |
| P16112 | Aggrecan core protein 2 | ACAN |
| P16233 | Pancreatic triacylglycerol lipase | PNLIP |
| P16442 | Histo-blood group ABO system transferase | ABO |
| P16471 | Prolactin receptor | PRLR |
| P16562 | Cysteine-rich secretory protein 2 | CRISP2 |
| P16619 | C-C motif chemokine 3-like 1 | CCL3L1 |
| P16860 | BNP(3-29) | NPPB |
| P16870 | Carboxypeptidase E | CPE |
| P16871 | Interleukin-7 receptor subunit alpha | IL7R |
| P17213 | Bactericidal permeability-increasing protein | BPI |
| P17538 | Chymotrypsinogen B | CTRB1 |
| P17931 | Galectin-3 | LGALS3 |
| P17936 | Insulin-like growth factor-binding protein 3 | IGFBP3 |
| P17948 | Vascular endothelial growth factor receptor 1 | FLT1 |
| P18065 | Insulin-like growth factor-binding protein 2 | IGFBP2 |
| P18075 | Bone morphogenetic protein 7 | BMP7 |
| P18428 | Lipopolysaccharide-binding protein | LBP |
| P18509 | PACAP-related peptide | ADCYAP1 |
| P18510 | Interleukin-1 receptor antagonist protein | IL1RN |
| P18827 | Syndecan-1 | SDC1 |
| P19021 | Peptidylglycine alpha-hydroxylating | PAM |
| | monooxygenase | |
| P19235 | Erythropoietin receptor | EPOR |
| P19438 | Tumor necrosis factor-binding protein 1 | TNFRSF1A |
| P19652 | Alpha-1-acid glycoprotein 2 | ORM2 |
| P19801 | Amiloride-sensitive amine oxidase [copper- | ABP1 |
| | containing] | |
| P19823 | Inter-alpha-trypsin inhibitor heavy chain H2 | ITIH2 |
| P19827 | Inter-alpha-trypsin inhibitor heavy chain H1 | ITIH1 |
| P19835 | Bile salt-activated lipase | CEL |
| P19875 | C-X-C motif chemokine 2 | CXCL2 |
| P19876 | C-X-C motif chemokine 3 | CXCL3 |
| P19883 | Follistatin | FST |
| P19957 | Elafin | PI3 |
| P19961 | Alpha-amylase 2B | AMY2B |
| P20061 | Transcobalamin-1 | TCN1 |
| P20062 | Transcobalamin-2 | TCN2 |
| P20142 | Gastricsin | PGC |
| P20155 | Serine protease inhibitor Kazal-type 2 | SPINK2 |
| P20231 | Tryptase beta-2 | TPSB2 |
| P20333 | Tumor necrosis factor receptor superfamily | TNFRSF1B |
| | member 1B | |
| P20366 | Substance P | TAC1 |
| P20382 | Melanin-concentrating hormone | PMCH |
| P20396 | Thyroliberin | TRH |
| P20742 | Pregnancy zone protein | PZP |
| P20774 | Mimecan | OGN |
| P20783 | Neurotrophin-3 | NTF3 |
| P20800 | Endothelin-2 | EDN2 |
| P20809 | Interleukin-11 | IL11 |
| P20827 | Ephrin-A1 | EFNA1 |
| P20849 | Collagen alpha-1(IX) chain | COL9A1 |
| P20851 | C4b-binding protein beta chain | C4BPB |
| P20908 | Collagen alpha-1(V) chain | COL5A1 |
| P21128 | Poly(U)-specific endoribonuclease | ENDOU |
| P21246 | Pleiotrophin | PTN |
| P21583 | Kit ligand | KITLG |
| P21741 | Midkine | MDK |
| P21754 | Zona pellucida sperm-binding protein 3 | ZP3 |
| P21781 | Fibroblast growth factor 7 | FGF7 |
| P21802 | Fibroblast growth factor receptor 2 | FGFR2 |
| P21810 | Biglycan | BGN |
| P21815 | Bone sialoprotein 2 | IBSP |
| P21860 | Receptor tyrosine-protein kinase erbB-3 | ERBB3 |
| P21941 | Cartilage matrix protein | MATN1 |
| P22003 | Bone morphogenetic protein 5 | BMP5 |
| P22004 | Bone morphogenetic protein 6 | BMP6 |
| P22079 | Lactoperoxidase | LPO |
| P22105 | Tenascin-X | TNXB |
| P22301 | Interleukin-10 | IL10 |
| P22303 | Acetylcholinesterase | ACHE |
| P22352 | Glutathione peroxidase 3 | GPX3 |
| P22362 | C-C motif chemokine 1 | CCL1 |
| P22455 | Fibroblast growth factor receptor 4 | FGFR4 |
| P22466 | Galanin message-associated peptide | GAL |
| P22692 | Insulin-like growth factor-binding protein 4 | IGFBP4 |
| P22749 | Granulysin | GNLY |
| P22792 | Carboxypeptidase N subunit 2 | CPN2 |
| P22891 | Vitamin K-dependent protein Z | PROZ |
| P22894 | Neutrophil collagenase | MMP8 |
| P23142 | Fibulin-1 | FBLN1 |
| P23280 | Carbonic anhydrase 6 | CA6 |
| P23352 | Anosmin-1 | KAL1 |
| P23435 | Cerebellin-1 | CBLN1 |
| P23560 | Brain-derived neurotrophic factor | BDNF |
| P23582 | C-type natriuretic peptide | NPPC |
| P23946 | Chymase | CMA1 |
| P24043 | Laminin subunit alpha-2 | LAMA2 |
| P24071 | Immunoglobulin alpha Fc receptor | FCAR |
| P24347 | Stromelysin-3 | MMP11 |
| P24387 | Corticotropin-releasing factor-binding protein | CRHBP |
| P24592 | Insulin-like growth factor-binding protein 6 | IGFBP6 |
| P24593 | Insulin-like growth factor-binding protein 5 | IGFBP5 |
| P24821 | Tenascin | TNC |
| P24855 | Deoxyribonuclease-1 | DNASE1 |
| P25067 | Collagen alpha-2(VIII) chain | COL8A2 |
| P25311 | Zinc-alpha-2-glycoprotein | AZGP1 |
| P25391 | Laminin subunit alpha-1 | LAMA1 |
| P25445 | Tumor necrosis factor receptor superfamily | FAS |
| | member 6 | |
| P25940 | Collagen alpha-3(V) chain | COL5A3 |
| P25942 | Tumor necrosis factor receptor superfamily | CD40 |
| | member 5 | |
| P26022 | Pentraxin-related protein PTX3 | PTX3 |
| P26927 | Hepatocyte growth factor-like protein beta chain | MST1 |
| P27169 | Serum paraoxonase/arylesterase 1 | PON1 |
| P27352 | Gastric intrinsic factor | GIF |
| P27487 | Dipeptidyl peptidase 4 membrane form | DPP4 |
| P27539 | Embryonic growth/differentiation factor 1 | GDF1 |
| P27658 | Vastatin | COL8A1 |
| P27797 | Calreticulin | CALR |
| P27918 | Properdin | CFP |
| P28039 | Acyloxyacyl hydrolase | AOAH |
| P28300 | Protein-lysine 6-oxidase | LOX |
| P28325 | Cystatin-D | CST5 |
| P28799 | Granulin-1 | GRN |
| P29122 | Proprotein convertase subtilisin/kexin type 6 | PCSK6 |
| P29279 | Connective tissue growth factor | CTGF |
| P29320 | Ephrin type-A receptor 3 | EPHA3 |
| P29400 | Collagen alpha-5(IV) chain | COL4A5 |
| P29459 | Interleukin-12 subunit alpha | IL12A |
| P29460 | Interleukin-12 subunit beta | IL12B |
| P29508 | Serpin B3 | SERPINB3 |
| P29622 | Kallistatin | SERPINA4 |
| P29965 | CD40 ligand, soluble form | CD40LG |
| P30990 | Neurotensin/neuromedin N | NTS |
| P31025 | Lipocalin-1 | LCN1 |
| P31151 | Protein S100-A7 | S100A7 |
| P31371 | Fibroblast growth factor 9 | FGF9 |
| P31431 | Syndecan-4 | SDC4 |
| P31947 | 14-3-3 protein sigma | SFN |
| P32455 | Interferon-induced guanylate-binding protein 1 | GBP1 |
| P32881 | Interferon alpha-8 | IFNA8 |
| P34096 | Ribonuclease 4 | RNASE4 |
| P34130 | Neurotrophin-4 | NTF4 |
| P34820 | Bone morphogenetic protein 8B | BMP8B |
| P35030 | Trypsin-3 | PRSS3 |
| P35052 | Secreted glypican-1 | GPC1 |
| P35070 | Betacellulin | BTC |
| P35225 | Interleukin-13 | IL13 |
| P35247 | Pulmonary surfactant-associated protein D | SFTPD |
| P35318 | ADM | ADM |
| P35542 | Serum amyloid A-4 protein | SAA4 |
| P35555 | Fibrillin-1 | FBN1 |
| P35556 | Fibrillin-2 | FBN2 |
| P35625 | Metalloproteinase inhibitor 3 | TIMP3 |
| P35858 | Insulin-like growth factor-binding protein complex | IGFALS |
| | acid labile subunit | |
| P35916 | Vascular endothelial growth factor receptor 3 | FLT4 |
| P35968 | Vascular endothelial growth factor receptor 2 | KDR |
| P36222 | Chitinase-3-like protein 1 | CHI3L1 |
| P36952 | Serpin B5 | SERPINB5 |
| P36955 | Pigment epithelium-derived factor | SERPINF1 |
| P36980 | Complement factor H-related protein 2 | CFHR2 |
| P39059 | Collagen alpha-1(XV) chain | COL15A1 |
| P39060 | Collagen alpha-1(XVIII) chain | COL18A1 |
| P39877 | Calcium-dependent phospholipase A2 | PLA2G5 |
| P39900 | Macrophage metalloelastase | MMP12 |
| P39905 | Glial cell line-derived neurotrophic factor | GDNF |
| P40225 | Thrombopoietin | THPO |
| P40967 | M-alpha | PMEL |
| P41159 | Leptin | LEP |
| P41221 | Protein Wnt-5a | WNT5A |
| P41222 | Prostaglandin-H2 D-isomerase | PTGDS |
| P41271 | Neuroblastoma suppressor of tumorigenicity 1 | NBL1 |
| P41439 | Folate receptor gamma | FOLR3 |
| P42127 | Agouti-signaling protein | ASIP |
| P42702 | Leukemia inhibitory factor receptor | LIFR |
| P42830 | ENA-78(9-78) | CXCL5 |
| P43026 | Growth/differentiation factor 5 | GDF5 |
| P43251 | Biotinidase | BTD |
| P43652 | Afamin | AFM |
| P45452 | Collagenase 3 | MMP13 |
| P47710 | Casoxin-D | CSN1S1 |
| P47929 | Galectin-7 | LGALS7B |
| P47972 | Neuronal pentraxin-2 | NPTX2 |
| P47989 | Xanthine oxidase | XDH |
| P47992 | Lymphotactin | XCL1 |
| P48023 | Tumor necrosis factor ligand superfamily | FASLG |
| | member 6, membrane form | |
| P48052 | Carboxypeptidase A2 | CPA2 |
| P48061 | Stromal cell-derived factor 1 | CXCL12 |
| P48304 | Lithostathine-1-beta | REG1B |
| P48307 | Tissue factor pathway inhibitor 2 | TFPI2 |
| P48357 | Leptin receptor | LEPR |
| P48594 | Serpin B4 | SERPINB4 |
| P48645 | Neuromedin-U-25 | NMU |
| P48740 | Mannan-binding lectin serine protease 1 | MASP1 |
| P48745 | Protein NOV homolog | NOV |
| P48960 | CD97 antigen subunit beta | CD97 |
| P49223 | Kunitz-type protease inhibitor 3 | SPINT3 |
| P49747 | Cartilage oligomeric matrix protein | COMP |
| P49763 | Placenta growth factor | PGF |
| P49765 | Vascular endothelial growth factor B | VEGFB |
| P49767 | Vascular endothelial growth factor C | VEGFC |
| P49771 | Fms-related tyrosine kinase 3 ligand | FLT3LG |
| P49862 | Kallikrein-7 | KLK7 |
| P49863 | Granzyme K | GZMK |
| P49908 | Selenoprotein P | SEPP1 |
| P49913 | Antibacterial protein FALL-39 | CAMP |
| P50607 | Tubby protein homolog | TUB |
| P51124 | Granzyme M | GZMM |
| P51512 | Matrix metalloproteinase-16 | MMP16 |
| P51654 | Glypican-3 | GPC3 |
| P51671 | Eotaxin | CCL11 |
| P51884 | Lumican | LUM |
| P51888 | Prolargin | PRELP |
| P52798 | Ephrin-A4 | EFNA4 |
| P52823 | Stanniocalcin-1 | STC1 |
| P53420 | Collagen alpha-4(IV) chain | COL4A4 |
| P53621 | Coatomer subunit alpha | COPA |
| P54108 | Cysteine-rich secretory protein 3 | CRISP3 |
| P54315 | Pancreatic lipase-related protein 1 | PNLIPRP1 |
| P54317 | Pancreatic lipase-related protein 2 | PNLIPRP2 |
| P54793 | Arylsulfatase F | ARSF |
| P55000 | Secreted Ly-6/uPAR-related protein 1 | SLURP1 |
| P55001 | Microfibrillar-associated protein 2 | MFAP2 |
| P55056 | Apolipoprotein C-IV | APOC4 |
| P55058 | Phospholipid transfer protein | PLTP |
| P55075 | Fibroblast growth factor 8 | FGF8 |
| P55081 | Microfibrillar-associated protein 1 | MFAP1 |
| P55083 | Microfibril-associated glycoprotein 4 | MFAP4 |
| P55107 | Bone morphogenetic protein 3B | GDF10 |
| P55145 | Mesencephalic astrocyte-derived neurotrophic | MANF |
| | factor | |
| P55259 | Pancreatic secretory granule membrane major | GP2 |
| | glycoprotein GP2 | |
| P55268 | Laminin subunit beta-2 | LAMB2 |
| P55773 | CCL23(30-99) | CCL23 |
| P55774 | C-C motif chemokine 18 | CCL18 |
| P55789 | FAD-linked sulfhydryl oxidase ALR | GFER |
| P56703 | Proto-oncogene Wnt-3 | WNT3 |
| P56704 | Protein Wnt-3a | WNT3A |
| P56705 | Protein Wnt-4 | WNT4 |
| P56706 | Protein Wnt-7b | WNT7B |
| P56730 | Neurotrypsin | PRSS12 |
| P56851 | Epididymal secretory protein E3-beta | EDDM3B |
| P56975 | Neuregulin-3 | NRG3 |
| P58062 | Serine protease inhibitor Kazal-type 7 | SPINK7 |
| P58215 | Lysyl oxidase homolog 3 | LOXL3 |
| P58294 | Prokineticin-1 | PROK1 |
| P58335 | Anthrax toxin receptor 2 | ANTXR2 |
| P58397 | A disintegrin and metalloproteinase with | ADAMTS12 |
| | thrombospondin motifs 12 | |
| P58417 | Neurexophilin-1 | NXPH1 |
| P58499 | Protein FAM3B | FAM3B |
| P59510 | A disintegrin and metalloproteinase with | ADAMTS20 |
| | thrombospondin motifs 20 | |
| P59665 | Neutrophil defensin 1 | DEFA1B |
| P59666 | Neutrophil defensin 3 | DEFA3 |
| P59796 | Glutathione peroxidase 6 | GPX6 |
| P59826 | BPI fold-containing family B member 3 | BPIFB3 |
| P59827 | BPI fold-containing family B member 4 | BPIFB4 |
| P59861 | Beta-defensin 131 | DEFB131 |
| P60022 | Beta-defensin 1 | DEFB1 |
| P60153 | Inactive ribonuclease-like protein 9 | RNASE9 |
| P60827 | Complement C1q tumor necrosis factor-related | C1QTNF8 |
| | protein 8 | |
| P60852 | Zona pellucida sperm-binding protein 1 | ZP1 |
| P60985 | Keratinocyte differentiation-associated protein | KRTDAP |
| P61109 | Kidney androgen-regulated protein | KAP |
| P61278 | Somatostatin-14 | SST |
| P61366 | Osteocrin | OSTN |
| P61626 | Lysozyme C | LYZ |
| P61769 | Beta-2-microglobulin | B2M |
| P61812 | Transforming growth factor beta-2 | TGFB2 |
| P61916 | Epididymal secretory protein E1 | NPC2 |
| P62502 | Epididymal-specific lipocalin-6 | LCN6 |
| P62937 | Peptidyl-prolyl cis-trans isomerase A | PPIA |
| P67809 | Nuclease-sensitive element-binding protein 1 | YBX1 |
| P67812 | Signal peptidase complex catalytic subunit | SEC11A |
| | SEC11A | |
| P78310 | Coxsackievirus and adenovirus receptor | CXADR |
| P78333 | Secreted glypican-5 | GPC5 |
| P78380 | Oxidized low-density lipoprotein receptor 1 | OLR1 |
| P78423 | Processed fractalkine | CX3CL1 |
| P78509 | Reelin | RELN |
| P78556 | CCL20(2-70) | CCL20 |
| P80075 | MCP-2(6-76) | CCL8 |
| P80098 | C-C motif chemokine 7 | CCL7 |
| P80108 | Phosphatidylinositol-glycan-specific | GPLD1 |
| | phospholipase D | |
| P80162 | C-X-C motif chemokine 6 | CXCL6 |
| P80188 | Neutrophil gelatinase-associated lipocalin | LCN2 |
| P80303 | Nucleobindin-2 | NUCB2 |
| P80511 | Calcitermin | S100A12 |
| P81172 | Hepcidin-25 | HAMP |
| P81277 | Prolactin-releasing peptide | PRLH |
| P81534 | Beta-defensin 103 | DEFB103A |
| P81605 | Dermcidin | DCD |
| P82279 | Protein crumbs homolog 1 | CRB1 |
| P82987 | ADAMTS-like protein 3 | ADAMTSL3 |
| P83105 | Serine protease HTRA4 | HTRA4 |
| P83110 | Serine protease HTRA3 | HTRA3 |
| P83859 | Orexigenic neuropeptide QRFP | QRFP |
| P98088 | Mucin-5AC | MUC5AC |
| P98095 | Fibulin-2 | FBLN2 |
| P98160 | Basement membrane-specific heparan sulfate | HSPG2 |
| | proteoglycan core protein | |
| P98173 | Protein FAM3A | FAM3A |
| Q00604 | Norrin | NDP |
| Q00796 | Sorbitol dehydrogenase | SORD |
| Q00887 | Pregnancy-specific beta-1-glycoprotein 9 | PSG9 |
| Q00888 | Pregnancy-specific beta-1-glycoprotein 4 | PSG4 |
| Q00889 | Pregnancy-specific beta-1-glycoprotein 6 | PSG6 |
| Q01523 | HD5(56-94) | DEFA5 |
| Q01524 | Defensin-6 | DEFA6 |
| Q01955 | Collagen alpha-3(IV) chain | COL4A3 |
| Q02297 | Pro-neuregulin-1, membrane-bound isoform | NRG1 |
| Q02325 | Plasminogen-like protein B | PLGLB1 |
| Q02383 | Semenogelin-2 | SEMG2 |
| Q02388 | Collagen alpha-1(VII) chain | COL7A1 |
| Q02505 | Mucin-3A | MUC3A |
| Q02509 | Otoconin-90 | OC90 |
| Q02747 | Guanylin | GUCA2A |
| Q02763 | Angiopoietin-1 receptor | TEK |
| Q02817 | Mucin-2 | MUC2 |
| Q02985 | Complement factor H-related protein 3 | CFHR3 |
| Q03167 | Transforming growth factor beta receptor type 3 | TGFBR3 |
| Q03403 | Trefoil factor 2 | TFF2 |
| Q03405 | Urokinase plasminogen activator surface receptor | PLAUR |
| Q03591 | Complement factor H-related protein 1 | CFHR1 |
| Q03692 | Collagen alpha-1(X) chain | COL10A1 |
| Q04118 | Basic salivary proline-rich protein 3 | PRB3 |
| Q04756 | Hepatocyte growth factor activator short chain | HGFAC |
| Q04900 | Sialomucin core protein 24 | CD164 |
| Q05315 | Eosinophil lysophospholipase | CLC |
| Q05707 | Collagen alpha-1(XIV) chain | COL14A1 |
| Q05996 | Processed zona pellucida sperm-binding protein 2 | ZP2 |
| Q06033 | Inter-alpha-trypsin inhibitor heavy chain H3 | ITIH3 |
| Q06141 | Regenerating islet-derived protein 3-alpha | REG3A |
| Q06828 | Fibromodulin | FMOD |
| Q07092 | Collagen alpha-1(XVI) chain | COL16A1 |
| Q07325 | C-X-C motif chemokine 9 | CXCL9 |
| Q07507 | Dermatopontin | DPT |
| Q075Z2 | Binder of sperm protein homolog 1 | BSPH1 |
| Q07654 | Trefoil factor 3 | TFF3 |
| Q07699 | Sodium channel subunit beta-1 | SCN1B |
| Q08345 | Epithelial discoidin domain-containing receptor 1 | DDR1 |
| Q08380 | Galectin-3-binding protein | LGALS3BP |
| Q08397 | Lysyl oxidase homolog 1 | LOXL1 |
| Q08431 | Lactadherin | MFGE8 |
| Q08629 | Testican-1 | SPOCK1 |
| Q08648 | Sperm-associated antigen 11B | SPAG11B |
| Q08830 | Fibrinogen-like protein 1 | FGL1 |
| Q10471 | Polypeptide N-acetylgalactosaminyltransferase 2 | GALNT2 |
| Q10472 | Polypeptide N-acetylgalactosaminyltransferase 1 | GALNT1 |
| Q11201 | CMP-N-acetylneuraminate-beta-galactosamide- | ST3GAL1 |
| | alpha-2,3-sialyltransferase 1 | |
| Q11203 | CMP-N-acetylneuraminate-beta-1,4-galactoside | ST3GAL3 |
| | alpha-2,3-sialyltransferase | |
| Q11206 | CMP-N-acetylneuraminate-beta-galactosamide- | ST3GAL4 |
| | alpha-2,3-sialyltransferase 4 | |
| Q12794 | Hyaluronidase-1 | HYAL1 |
| Q12805 | EGF-containing fibulin-like extracellular matrix | EFEMP1 |
| | protein 1 | |
| Q12836 | Zona pellucida sperm-binding protein 4 | ZP4 |
| Q12841 | Follistatin-related protein 1 | FSTL1 |
| Q12904 | Aminoacyl tRNA synthase complex-interacting | AIMP1 |
| | multifunctional protein 1 | |
| Q13018 | Soluble secretory phospholipase A2 receptor | PLA2R1 |
| Q13072 | B melanoma antigen 1 | BAGE |
| Q13093 | Platelet-activating factor acetylhydrolase | PLA2G7 |
| Q13103 | Secreted phosphoprotein 24 | SPP2 |
| Q13162 | Peroxiredoxin-4 | PRDX4 |
| Q13201 | Platelet glycoprotein Ia* | MMRN1 |
| Q13214 | Semaphorin-3B | SEMA3B |
| Q13219 | Pappalysin-1 | PAPPA |
| Q13231 | Chitotriosidase-1 | CHIT1 |
| Q13253 | Noggin | NOG |
| Q13261 | Interleukin-15 receptor subunit alpha | IL15RA |
| Q13275 | Semaphorin-3F | SEMA3F |
| Q13291 | Signaling lymphocytic activation molecule | SLAMF1 |
| Q13316 | Dentin matrix acidic phosphoprotein 1 | DMP1 |
| Q13361 | Microfibrillar-associated protein 5 | MFAP5 |
| Q13410 | Butyrophilin subfamily 1 member A1 | BTN1A1 |
| Q13421 | Mesothelin, cleaved form | MSLN |
| Q13429 | Insulin-like growth factor I | IGF-I |
| Q13443 | Disintegrin and metalloproteinase domain- | ADAM9 |
| | containing protein 9 | |
| Q13519 | Neuropeptide 1 | PNOC |
| Q13751 | Laminin subunit beta-3 | LAMB3 |
| Q13753 | Laminin subunit gamma-2 | LAMC2 |
| Q13790 | Apolipoprotein F | APOF |
| Q13822 | Ectonucleotide pyrophosphatase/phosphodiesterase | ENPP2 |
| | family member 2 | |
| Q14031 | Collagen alpha-6(IV) chain | COL4A6 |
| Q14050 | Collagen alpha-3(IX) chain | COL9A3 |
| Q14055 | Collagen alpha-2(IX) chain | COL9A2 |
| Q14112 | Nidogen-2 | NID2 |
| Q14114 | Low-density lipoprotein receptor-related protein 8 | LRP8 |
| Q14118 | Dystroglycan | DAG1 |
| Q14314 | Fibroleukin | FGL2 |
| Q14393 | Growth arrest-specific protein 6 | GAS6 |
| Q14406 | Chorionic somatomammotropin hormone-like 1 | CSHL1 |
| Q14507 | Epididymal secretory protein E3-alpha | EDDM3A |
| Q14508 | WAP four-disulfide core domain protein 2 | WFDC2 |
| Q14512 | Fibroblast growth factor-binding protein 1 | FGFBP1 |
| Q14515 | SPARC-like protein 1 | SPARCL1 |
| Q14520 | Hyaluronan-binding protein 2 27 kDa light chain | HABP2 |
| Q14563 | Semaphorin-3A | SEMA3A |
| Q14623 | Indian hedgehog protein | IHH |
| Q14624 | Inter-alpha-trypsin inhibitor heavy chain H4 | ITIH4 |
| Q14667 | UPF0378 protein KIAA0100 | KIAA0100 |
| Q14703 | Membrane-bound transcription factor site-1 | MBTPS1 |
| | protease | |
| Q14766 | Latent-transforming growth factor beta-binding | LTBP1 |
| | protein 1 | |
| Q14767 | Latent-transforming growth factor beta-binding | LTBP2 |
| | protein 2 | |
| Q14773 | Intercellular adhesion molecule 4 | ICAM4 |
| Q14993 | Collagen alpha-1(XIX) chain | COL19A1 |
| Q14CN2 | Calcium-activated chloride channel regulator 4, | CLCA4 |
| | 110 kDa form | |
| Q15046 | Lysine--tRNA ligase | KARS |
| Q15063 | Periostin | POSTN |
| Q15109 | Advanced glycosylation end product-specific | AGER |
| | receptor | |
| Q15113 | Procollagen C-endopeptidase enhancer 1 | PCOLCE |
| Q15166 | Serum paraoxonase/lactonase 3 | PON3 |
| Q15195 | Plasminogen-like protein A | PLGLA |
| Q15198 | Platelet-derived growth factor receptor-like protein | PDGFRL |
| Q15223 | Poliovirus receptor-related protein 1 | PVRL1 |
| Q15238 | Pregnancy-specific beta-1-glycoprotein 5 | PSG5 |
| Q15363 | Transmembrane emp24 domain-containing protein 2 | TMED2 |
| Q15375 | Ephrin type-A receptor 7 | EPHA7 |
| Q15389 | Angiopoietin-1 | ANGPT1 |
| Q15465 | Sonic hedgehog protein | SHH |
| Q15485 | Ficolin-2 | FCN2 |
| Q15517 | Corneodesmosin | CDSN |
| Q15582 | Transforming growth factor-beta-induced protein ig-h3 | TGFBI |
| Q15661 | Tryptase alpha/beta-1 | TPSAB1 |
| Q15726 | Metastin | KISS1 |
| Q15782 | Chitinase-3-like protein 2 | CHI3L2 |
| Q15828 | Cystatin-M | CST6 |
| Q15846 | Clusterin-like protein 1 | CLUL1 |
| Q15848 | Adiponectin | ADIPOQ |
| Q16206 | Protein disulfide-thiol oxidoreductase | ENOX2 |
| Q16270 | Insulin-like growth factor-binding protein 7 | IGFBP7 |
| Q16363 | Laminin subunit alpha-4 | LAMA4 |
| Q16378 | Proline-rich protein 4 | PRR4 |
| Q16557 | Pregnancy-specific beta-1-glycoprotein 3 | PSG3 |
| Q16568 | CART(42-89) | CARTPT |
| Q16610 | Extracellular matrix protein 1 | ECM1 |
| Q16619 | Cardiotrophin-1 | CTF1 |
| Q16623 | Syntaxin-1A | STX1A |
| Q16627 | HCC-1(9-74) | CCL14 |
| Q16651 | Prostasin light chain | PRSS8 |
| Q16661 | Guanylate cyclase C-activating peptide 2 | GUCA2B |
| Q16663 | CCL15(29-92) | CCL15 |
| Q16674 | Melanoma-derived growth regulatory protein | MIA |
| Q16769 | Glutaminyl-peptide cyclotransferase | QPCT |
| Q16787 | Laminin subunit alpha-3 | LAMA3 |
| Q16842 | CMP-N-acetylneuraminate-beta-galactosamide- | ST3GAL2 |
| | alpha-2,3-sialyltransferase 2 | |
| Q17RR3 | Pancreatic lipase-related protein 3 | PNLIPRP3 |
| Q17RW2 | Collagen alpha-1(XXIV) chain | COL24A1 |
| Q17RY6 | Lymphocyte antigen 6K | LY6K |
| Q1L6U9 | Prostate-associated microseminoprotein | MSMP |
| Q1W4C9 | Serine protease inhibitor Kazal-type 13 | SPINK13 |
| Q1ZYL8 | Izumo sperm-egg fusion protein 4 | IZUMO4 |
| Q29960 | HLA class I histocompatibility antigen, Cw-16 | HLA-C |
| | alpha chain | |
| Q2I0M5 | R-spondin-4 | RSPO4 |
| Q2L4Q9 | Serine protease 53 | PRSS53 |
| Q2MKA7 | R-spondin-1 | RSPO1 |
| Q2MV58 | Tectonic-1 | TCTN1 |
| Q2TAL6 | Brorin | VWC2 |
| Q2UY09 | Collagen alpha-1(XXVIII) chain | COL28A1 |
| Q2VPA4 | Complement component receptor 1-like protein | CR1L |
| Q2WEN9 | Carcinoembryonic antigen-related cell adhesion | CEACAM16 |
| | molecule 16 | |
| Q30KP8 | Beta-defensin 136 | DEFB136 |
| Q30KP9 | Beta-defensin 135 | DEFB135 |
| Q30KQ1 | Beta-defensin 133 | DEFB133 |
| Q30KQ2 | Beta-defensin 130 | DEFB130 |
| Q30KQ4 | Beta-defensin 116 | DEFB116 |
| Q30KQ5 | Beta-defensin 115 | DEFB115 |
| Q30KQ6 | Beta-defensin 114 | DEFB114 |
| Q30KQ7 | Beta-defensin 113 | DEFB113 |
| Q30KQ8 | Beta-defensin 112 | DEFB112 |
| Q30KQ9 | Beta-defensin 110 | DEFB110 |
| Q30KR1 | Beta-defensin 109 | DEFB109P1 |
| Q32P28 | Prolyl 3-hydroxylase 1 | LEPRE1 |
| Q3B7J2 | Glucose-fructose oxidoreductase domain- | GFOD2 |
| | containing protein 2 | |
| Q3SY79 | Protein Wnt | WNT3A |
| Q3T906 | N-acetylglucosamine-1-phosphotransferase | GNPTAB |
| | subunits alpha/beta | |
| Q495T6 | Membrane metallo-endopeptidase-like 1 | MMEL1 |
| Q49AH0 | Cerebral dopamine neurotrophic factor | CDNF |
| Q4G0G5 | Secretoglobin family 2B member 2 | SCGB2B2 |
| Q4G0M1 | Protein FAM132B | FAM132B |
| Q4LDE5 | Sushi, von Willebrand factor type A, EGF and | SVEP1 |
| | pentraxin domain-containing protein 1 | |
| Q4QY38 | Beta-defensin 134 | DEFB134 |
| Q4VAJ4 | Protein Wnt | WNT10B |
| Q4W5P6 | Protein TMEM155 | TMEM155 |
| Q4ZHG4 | Fibronectin type III domain-containing protein 1 | FNDC1 |
| Q53H76 | Phospholipase A1 member A | PLA1A |
| Q53RD9 | Fibulin-7 | FBLN7 |
| Q53S33 | BolA-like protein 3 | BOLA3 |
| Q5BLP8 | Neuropeptide-like protein C4orf48 | C4orf48 |
| Q5DT21 | Serine protease inhibitor Kazal-type 9 | SPINK9 |
| Q5EBL8 | PDZ domain-containing protein 11 | PDZD11 |
| Q5FYB0 | Arylsulfatase J | ARSJ |
| Q5FYB1 | Arylsulfatase I | ARSI |
| Q5GAN3 | Ribonuclease-like protein 13 | RNASE13 |
| Q5GAN4 | Ribonuclease-like protein 12 | RNASE12 |
| Q5GAN6 | Ribonuclease-like protein 10 | RNASE10 |
| Q5GFL6 | von Willebrand factor A domain-containing | VWA2 |
| | protein 2 | |
| Q5H8A3 | Neuromedin-S | NMS |
| Q5H8C1 | FRAS1-related extracellular matrix protein 1 | FREM1 |
| Q5IJ48 | Protein crumbs homolog 2 | CRB2 |
| Q5J5C9 | Beta-defensin 121 | DEFB121 |
| Q5JS37 | NHL repeat-containing protein 3 | NHLRC3 |
| Q5JTB6 | Placenta-specific protein 9 | PLAC9 |
| Q5JU69 | Torsin-2A | TOR2A |
| Q5JXM2 | Methyltransferase-like protein 24 | METTL24 |
| Q5JZY3 | Ephrin type-A receptor 10 | EPHA10 |
| Q5K4E3 | Polyserase-2 | PRSS36 |
| Q5SRR4 | Lymphocyte antigen 6 complex locus protein G5c | LY6G5C |
| Q5T1H1 | Protein eyes shut homolog | EYS |
| Q5T4F7 | Secreted frizzled-related protein 5 | SFRP5 |
| Q5T4W7 | Artemin | ARTN |
| Q5T7M4 | Protein FAM132A | FAM132A |
| Q5TEH8 | Protein Wnt | WNT2B |
| Q5TIE3 | von Willebrand factor A domain-containing | VWA5B1 |
| | protein 5B1 | |
| Q5UCC4 | ER membrane protein complex subunit 10 | EMC10 |
| Q5VST6 | Abhydrolase domain-containing protein | FAM108B1 |
| | FAM108B1 | |
| Q5VTL7 | Fibronectin type III domain-containing protein 7 | FNDC7 |
| Q5VUM1 | UPF0369 protein C6orf57 | C6orf57 |
| Q5VV43 | Dyslexia-associated protein KIAA0319 | KIAA0319 |
| Q5VWW1 | Complement C1q-like protein 3 | C1QL3 |
| Q5VXI9 | Lipase member N | LIPN |
| Q5VXJ0 | Lipase member K | LIPK |
| Q5VXM1 | CUB domain-containing protein 2 | CDCP2 |
| Q5VYX0 | Renalase | RNLS |
| Q5VYY2 | Lipase member M | LIPM |
| Q5W186 | Cystatin-9 | CST9 |
| Q5W5W9 | Regulated endocrine-specific protein 18 | RESP18 |
| Q5XG92 | Carboxylesterase 4A | CES4A |
| Q63HQ2 | Pikachurin | EGFLAM |
| Q641Q3 | Meteorin-like protein | METRNL |
| Q66K79 | Carboxypeptidase Z | CPZ |
| Q685J3 | Mucin-17 | MUC17 |
| Q68BL7 | Olfactomedin-like protein 2A | OLFML2A |
| Q68BL8 | Olfactomedin-like protein 2B | OLFML2B |
| Q68DV7 | E3 ubiquitin-protein ligase RNF43 | RNF43 |
| Q6B9Z1 | Insulin growth factor-like family member 4 | IGFL4 |
| Q6BAA4 | Fc receptor-like B | FCRLB |
| Q6E0U4 | Dermokine | DMKN |
| Q6EMK4 | Vasorin | VASN |
| Q6FHJ7 | Secreted frizzled-related protein 4 | SFRP4 |
| Q6GPI1 | Chymotrypsin B2 chain B | CTRB2 |
| Q6GTS8 | Probable carboxypeptidase PM20D1 | PM20D1 |
| Q6H9L7 | Isthmin-2 | ISM2 |
| Q6IE36 | Ovostatin homolog 2 | OVOS2 |
| Q6IE37 | Ovostatin homolog 1 | OVOS1 |
| Q6IE38 | Serine protease inhibitor Kazal-type 14 | SPINK14 |
| Q6ISS4 | Leukocyte-associated immunoglobulin-like | LAIR2 |
| | receptor 2 | |
| Q6JVE5 | Epididymal-specific lipocalin-12 | LCN12 |
| Q6JVE6 | Epididymal-specific lipocalin-10 | LCN10 |
| Q6JVE9 | Epididymal-specific lipocalin-8 | LCN8 |
| Q6KF10 | Growth/differentiation factor 6 | GDF6 |
| Q6MZW2 | Follistatin-related protein 4 | FSTL4 |
| Q6NSX1 | Coiled-coil domain-containing protein 70 | CCDC70 |
| Q6NT32 | Carboxylesterase 5A | CES5A |
| Q6NT52 | Choriogonadotropin subunit beta variant 2 | CGB2 |
| Q6NUI6 | Chondroadherin-like protein | CHADL |
| Q6NUJ1 | Saposin A-like | PSAPL1 |
| Q6P093 | Arylacetamide deacetylase-like 2 | AADACL2 |
| Q6P4A8 | Phospholipase B-like 1 | PLBD1 |
| Q6P5S2 | UPF0762 protein C6orf58 | C6orf58 |
| Q6P988 | Protein notum homolog | NOTUM |
| Q6PCB0 | von Willebrand factor A domain-containing | VWA1 |
| | protein 1 | |
| Q6PDA7 | Sperm-associated antigen 11A | SPAG11A |
| Q6PEW0 | Inactive serine protease 54 | PRSS54 |
| Q6PEZ8 | Podocan-like protein 1 | PODNL1 |
| Q6PKH6 | Dehydrogenase/reductase SDR family member 4- | DHRS4L2 |
| | like 2 | |
| Q6Q788 | Apolipoprotein A-V | APOA5 |
| Q6SPF0 | Atherin | SAMD1 |
| Q6UDR6 | Kunitz-type protease inhibitor 4 | SPINT4 |
| Q6URK8 | Testis, prostate and placenta-expressed protein | TEPP |
| Q6UW01 | Cerebellin-3 | CBLN3 |
| Q6UW10 | Surfactant-associated protein 2 | SFTA2 |
| Q6UW15 | Regenerating islet-derived protein 3-gamma | REG3G |
| Q6UW32 | Insulin growth factor-like family member 1 | IGFL1 |
| Q6UW78 | UPF0723 protein C11orf83 | C11orf83 |
| Q6UW88 | Epigen | EPGN |
| Q6UWE3 | Colipase-like protein 2 | CLPSL2 |
| Q6UWF7 | NXPE family member 4 | NXPE4 |
| Q6UWF9 | Protein FAM180A | FAM180A |
| Q6UWM5 | GLIPR1-like protein 1 | GLIPR1L1 |
| Q6UWN8 | Serine protease inhibitor Kazal-type 6 | SPINK6 |
| Q6UWP2 | Dehydrogenase/reductase SDR family member 11 | DHRS11 |
| Q6UWP8 | Suprabasin | SBSN |
| Q6UWQ5 | Lysozyme-like protein 1 | LYZL1 |
| Q6UWQ7 | Insulin growth factor-like family member 2 | IGFL2 |
| Q6UWR7 | Ectonucleotide pyrophosphatase/phosphodiesterase | ENPP6 |
| | family member 6 soluble form | |
| Q6UWT2 | Adropin | ENHO |
| Q6UWU2 | Beta-galactosidase-1-like protein | GLB1L |
| Q6UWW0 | Lipocalin-15 | LCN15 |
| Q6UWX4 | HHIP-like protein 2 | HHIPL2 |
| Q6UWY0 | Arylsulfatase K | ARSK |
| Q6UWY2 | Serine protease 57 | PRSS57 |
| Q6UWY5 | Olfactomedin-like protein 1 | OLFML1 |
| Q6UX06 | Olfactomedin-4 | OLFM4 |
| Q6UX07 | Dehydrogenase/reductase SDR family member 13 | DHRS13 |
| Q6UX39 | Amelotin | AMTN |
| Q6UX46 | Protein FAM150B | FAM150B |
| Q6UX73 | UPF0764 protein C16orf89 | C16orf89 |
| Q6UXB0 | Protein FAM131A | FAM131A |
| Q6UXB1 | Insulin growth factor-like family member 3 | IGFL3 |
| Q6UXB2 | VEGF co-regulated chemokine 1 | CXCL17 |
| Q6UXF7 | C-type lectin domain family 18 member B | CLEC18B |
| Q6UXH0 | Hepatocellular carcinoma-associated protein TD26 | C19orf80 |
| Q6UXH1 | Cysteine-rich with EGF-like domain protein 2 | CRELD2 |
| Q6UXH8 | Collagen and calcium-binding EGF domain- | CCBE1 |
| | containing protein 1 | |
| Q6UXH9 | Inactive serine protease PAMR1 | PAMR1 |
| Q6UXI7 | Vitrin | VIT |
| Q6UXI9 | Nephronectin | NPNT |
| Q6UXN2 | Trem-like transcript 4 protein | TREML4 |
| Q6UXS0 | C-type lectin domain family 19 member A | CLEC19A |
| Q6UXT8 | Protein FAM150A | FAM150A |
| Q6UXT9 | Abhydrolase domain-containing protein 15 | ABHD15 |
| Q6UXV4 | Apolipoprotein O-like | APOOL |
| Q6UXX5 | Inter-alpha-trypsin inhibitor heavy chain H6 | ITIH6 |
| Q6UXX9 | R-spondin-2 | RSPO2 |
| Q6UY14 | ADAMTS-like protein 4 | ADAMTSL4 |
| Q6UY27 | Prostate and testis expressed protein 2 | PATE2 |
| Q6W4X9 | Mucin-6 | MUC6 |
| Q6WN34 | Chordin-like protein 2 | CHRDL2 |
| Q6WRI0 | Immunoglobulin superfamily member 10 | IGSF10 |
| Q6X4U4 | Sclerostin domain-containing protein 1 | SOSTDC1 |
| Q6X784 | Zona pellucida-binding protein 2 | ZPBP2 |
| Q6XE38 | Secretoglobin family 1D member 4 | SCGB1D4 |
| Q6XPR3 | Repetin | RPTN |
| Q6XZB0 | Lipase member I | LIPI |
| Q6ZMM2 | ADAMTS-like protein 5 | ADAMTSL5 |
| Q6ZMP0 | Thrombospondin type-1 domain-containing | THSD4 |
| | protein 4 | |
| Q6ZNF0 | Iron/zinc purple acid phosphatase-like protein | PAPL |
| Q6ZRI0 | Otogelin | OTOG |
| Q6ZRP7 | Sulfhydryl oxidase 2 | QSOX2 |
| Q6ZWJ8 | Kielin/chordin-like protein | KCP |
| Q75N90 | Fibrillin-3 | FBN3 |
| Q765I0 | Urotensin-2B | UTS2D |
| Q76B58 | Protein FAM5C | FAM5C |
| Q76LX8 | A disintegrin and metalloproteinase with | ADAMTS13 |
| | thrombospondin motifs 13 | |
| Q76M96 | Coiled-coil domain-containing protein 80 | CCDC80 |
| Q7L1S5 | Carbohydrate sulfotransferase 9 | CHST9 |
| Q7L513 | Fc receptor-like A | FCRLA |
| Q7L8A9 | Vasohibin-1 | VASH1 |
| Q7RTM1 | Otopetrin-1 | OTOP1 |
| Q7RTW8 | Otoancorin | OTOA |
| Q7RTY5 | Serine protease 48 | PRSS48 |
| Q7RTY7 | Ovochymase-1 | OVCH1 |
| Q7RTZ1 | Ovochymase-2 | OVCH2 |
| Q7Z304 | MAM domain-containing protein 2 | MAMDC2 |
| Q7Z3S9 | Notch homolog 2 N-terminal-like protein | NOTCH2NL |
| Q7Z4H4 | Intermedin-short | ADM2 |
| Q7Z4P5 | Growth/differentiation factor 7 | GDF7 |
| Q7Z4R8 | UPF0669 protein C6orf120 | C6orf120 |
| Q7Z4W2 | Lysozyme-like protein 2 | LYZL2 |
| Q7Z5A4 | Serine protease 42 | PRSS42 |
| Q7Z5A7 | Protein FAM19A5 | FAM19A5 |
| Q7Z5A8 | Protein FAM19A3 | FAM19A3 |
| Q7Z5A9 | Protein FAM19A1 | FAM19A1 |
| Q7Z5J1 | Hydroxysteroid 11-beta-dehydrogenase 1-like | HSD11B1L |
| | protein | |
| Q7Z5L0 | Vitelline membrane outer layer protein 1 homolog | VMO1 |
| Q7Z5L3 | Complement C1q-like protein 2 | C1QL2 |
| Q7Z5L7 | Podocan | PODN |
| Q7Z5P4 | 17-beta-hydroxysteroid dehydrogenase 13 | HSD17B13 |
| Q7Z5P9 | Mucin-19 | MUC19 |
| Q7Z5Y6 | Bone morphogenetic protein 8A | BMP8A |
| Q7Z7B7 | Beta-defensin 132 | DEFB132 |
| Q7Z7B8 | Beta-defensin 128 | DEFB128 |
| Q7Z7C8 | Transcription initiation factor TFIID subunit 8 | TAF8 |
| Q7Z7H5 | Transmembrane emp24 domain-containing protein 4 | TMED4 |
| Q86SG7 | Lysozyme g-like protein 2 | LYG2 |
| Q86SI9 | Protein CEI | C5orf38 |
| Q86TE4 | Leucine zipper protein 2 | LUZP2 |
| Q86TH1 | ADAMTS-like protein 2 | ADAMTSL2 |
| Q86U17 | Serpin A11 | SERPINA11 |
| Q86UU9 | Endokinin-A | TAC4 |
| Q86UW8 | Hyaluronan and proteoglycan link protein 4 | HAPLN4 |
| Q86UX2 | Inter-alpha-trypsin inhibitor heavy chain H5 | ITIH5 |
| Q86V24 | Adiponectin receptor protein 2 | ADIPOR2 |
| Q86VB7 | Soluble CD163 | CD163 |
| Q86VR8 | Four-jointed box protein 1 | FJX1 |
| Q86WD7 | Serpin A9 | SERPINA9 |
| Q86WN2 | Interferon epsilon | IFNE |
| Q86WS3 | Placenta-specific 1-like protein | PLAC1L |
| Q86X52 | Chondroitin sulfate synthase 1 | CHSY1 |
| Q86XP6 | Gastrokine-2 | GKN2 |
| Q86XS5 | Angiopoietin-related protein 5 | ANGPTL5 |
| Q86Y27 | B melanoma antigen 5 | BAGE5 |
| Q86Y28 | B melanoma antigen 4 | BAGE4 |
| Q86Y29 | B melanoma antigen 3 | BAGE3 |
| Q86Y30 | B melanoma antigen 2 | BAGE2 |
| Q86Y38 | Xylosyltransferase 1 | XYLT1 |
| Q86Y78 | Ly6/PLAUR domain-containing protein 6 | LYPD6 |
| Q86YD3 | Transmembrane protein 25 | TMEM25 |
| Q86YJ6 | Threonine synthase-like 2 | THNSL2 |
| Q86YW7 | Glycoprotein hormone beta-5 | GPHB5 |
| Q86Z23 | Complement C1q-like protein 4 | C1QL4 |
| Q8IU57 | Interleukin-28 receptor subunit alpha | IL28RA |
| Q8IUA0 | WAP four-disulfide core domain protein 8 | WFDC8 |
| Q8IUB2 | WAP four-disulfide core domain protein 3 | WFDC3 |
| Q8IUB3 | Protein WFDC10B | WFDC10B |
| Q8IUB5 | WAP four-disulfide core domain protein 13 | WFDC13 |
| Q8IUH2 | Protein CREG2 | CREG2 |
| Q8IUK5 | Plexin domain-containing protein 1 | PLXDC1 |
| Q8IUL8 | Cartilage intermediate layer protein 2 C2 | CILP2 |
| Q8IUX7 | Adipocyte enhancer-binding protein 1 | AEBP1 |
| Q8IUX8 | Epidermal growth factor-like protein 6 | EGFL6 |
| Q8IVL8 | Carboxypeptidase O | CPO |
| Q8IVN8 | Somatomedin-B and thrombospondin type-1 | SBSPON |
| | domain-containing protein | |
| Q8IVW8 | Protein spinster homolog 2 | SPNS2 |
| Q8IW75 | Serpin A12 | SERPINA12 |
| Q8IW92 | Beta-galactosidase-1-like protein 2 | GLB1L2 |
| Q8IWL1 | Pulmonary surfactant-associated protein A2 | SFTPA2 |
| Q8IWL2 | Pulmonary surfactant-associated protein A1 | SFTPA1 |
| Q8IWV2 | Contactin-4 | CNTN4 |
| Q8IWY4 | Signal peptide, CUB and EGF-like domain- | SCUBE1 |
| | containing protein 1 | |
| Q8IX30 | Signal peptide, CUB and EGF-like domain- | SCUBE3 |
| | containing protein 3 | |
| Q8IXA5 | Sperm acrosome membrane-associated protein 3, | SPACA3 |
| | membrane form | |
| Q8IXB1 | DnaJ homolog subfamily C member 10 | DNAJC10 |
| Q8IXL6 | Extracellular serine/threonine protein kinase | FAM20C |
| | Fam20C | |
| Q8IYD9 | Lung adenoma susceptibility protein 2 | LAS2 |
| Q8IYP2 | Serine protease 58 | PRSS58 |
| Q8IYS5 | Osteoclast-associated immunoglobulin-like | OSCAR |
| | receptor | |
| Q8IZC6 | Collagen alpha-1(XXVII) chain | COL27A1 |
| Q8IZJ3 | C3 and PZP-like alpha-2-macroglobulin domain- | CPAMD8 |
| | containing protein 8 | |
| Q8IZN7 | Beta-defensin 107 | DEFB107B |
| Q8N0V4 | Leucine-rich repeat LGI family member 2 | LGI2 |
| Q8N104 | Beta-defensin 106 | DEFB106B |
| Q8N119 | Matrix metalloproteinase-21 | MMP21 |
| Q8N129 | Protein canopy homolog 4 | CNPY4 |
| Q8N135 | Leucine-rich repeat LGI family member 4 | LGI4 |
| Q8N145 | Leucine-rich repeat LGI family member 3 | LGI3 |
| Q8N158 | Glypican-2 | GPC2 |
| Q8N1E2 | Lysozyme g-like protein 1 | LYG1 |
| Q8N2E2 | von Willebrand factor D and EGF domain- | VWDE |
| | containing protein | |
| Q8N2E6 | Prosalusin | TOR2A |
| Q8N2S1 | Latent-transforming growth factor beta-binding | LTBP4 |
| | protein 4 | |
| Q8N302 | Angiogenic factor with G patch and FHA domains 1 | AGGF1 |
| Q8N307 | Mucin-20 | MUC20 |
| Q8N323 | NXPE family member 1 | NXPE1 |
| Q8N387 | Mucin-15 | MUC15 |
| Q8N3Z0 | Inactive serine protease 35 | PRSS35 |
| Q8N436 | Inactive carboxypeptidase-like protein X2 | CPXM2 |
| Q8N474 | Secreted frizzled-related protein 1 | SFRP1 |
| Q8N475 | Follistatin-related protein 5 | FSTL5 |
| Q8N4F0 | BPI fold-containing family B member 2 | BPIFB2 |
| Q8N4T0 | Carboxypeptidase A6 | CPA6 |
| Q8N5W8 | Protein FAM24B | FAM24B |
| Q8N687 | Beta-defensin 125 | DEFB125 |
| Q8N688 | Beta-defensin 123 | DEFB123 |
| Q8N690 | Beta-defensin 119 | DEFB119 |
| Q8N6C5 | Immunoglobulin superfamily member 1 | IGSF1 |
| Q8N6C8 | Leukocyte immunoglobulin-like receptor | LILRA3 |
| | subfamily A member 3 | |
| Q8N6G6 | ADAMTS-like protein 1 | ADAMTSL1 |
| Q8N6Y2 | Leucine-rich repeat-containing protein 17 | LRRC17 |
| Q8N729 | Neuropeptide W-23 | NPW |
| Q8N8U9 | BMP-binding endothelial regulator protein | BMPER |
| Q8N907 | DAN domain family member 5 | DAND5 |
| Q8NAT1 | Glycosyltransferase-like domain-containing | GTDC2 |
| | protein 2 | |
| Q8NAU1 | Fibronectin type III domain-containing protein 5 | FNDC5 |
| Q8NB37 | Parkinson disease 7 domain-containing protein 1 | PDDC1 |
| Q8NBI3 | Draxin | DRAXIN |
| Q8NBM8 | Prenylcysteine oxidase-like | PCYOX1L |
| Q8NBP7 | Proprotein convertase subtilisin/kexin type 9 | PCSK9 |
| Q8NBQ5 | Estradiol 17-beta-dehydrogenase 11 | HSD17B11 |
| Q8NBV8 | Synaptotagmin-8 | SYT8 |
| Q8NCC3 | Group XV phospholipase A2 | PLA2G15 |
| Q8NCF0 | C-type lectin domain family 18 member C | CLEC18C |
| Q8NCW5 | NAD(P)H-hydrate epimerase | APOA1BP |
| Q8NDA2 | Hemicentin-2 | HMCN2 |
| Q8NDX9 | Lymphocyte antigen 6 complex locus protein G5b | LY6G5B |
| Q8NDZ4 | Deleted in autism protein 1 | C3orf58 |
| Q8NEB7 | Acrosin-binding protein | ACRBP |
| Q8NES8 | Beta-defensin 124 | DEFB124 |
| Q8NET1 | Beta-defensin 108B | DEFB108B |
| Q8NEX5 | Protein WFDC9 | WFDC9 |
| Q8NEX6 | Protein WFDC11 | WFDC11 |
| Q8NF86 | Serine protease 33 | PRSS33 |
| Q8NFM7 | Interleukin-17 receptor D | IL17RD |
| Q8NFQ5 | BPI fold-containing family B member 6 | BPIFB6 |
| Q8NFQ6 | BPI fold-containing family C protein | BPIFC |
| Q8NFU4 | Follicular dendritic cell secreted peptide | FDCSP |
| Q8NFW1 | Collagen alpha-1(XXII) chain | COL22A1 |
| Q8NG35 | Beta-defensin 105 | DEFB105B |
| Q8NG41 | Neuropeptide B-23 | NPB |
| Q8NHW6 | Otospiralin | OTOS |
| Q8NI99 | Angiopoietin-related protein 6 | ANGPTL6 |
| Q8TAA1 | Probable ribonuclease 11 | RNASE11 |
| Q8TAG5 | V-set and transmembrane domain-containing | VSTM2A |
| | protein 2A | |
| Q8TAL6 | Fin bud initiation factor homolog | FIBIN |
| Q8TAT2 | Fibroblast growth factor-binding protein 3 | FGFBP3 |
| Q8TAX7 | Mucin-7 | MUC7 |
| Q8TB22 | Spermatogenesis-associated protein 20 | SPATA20 |
| Q8TB73 | Protein NDNF | NDNF |
| Q8TB96 | T-cell immunomodulatory protein | ITFG1 |
| Q8TC92 | Protein disulfide-thiol oxidoreductase | ENOX1 |
| Q8TCV5 | WAP four-disulfide core domain protein 5 | WFDC5 |
| Q8TD06 | Anterior gradient protein 3 homolog | AGR3 |
| Q8TD33 | Secretoglobin family 1C member 1 | SCGB1C1 |
| Q8TD46 | Cell surface glycoprotein CD200 receptor 1 | CD200R1 |
| Q8TDE3 | Ribonuclease 8 | RNASE8 |
| Q8TDF5 | Neuropilin and tolloid-like protein 1 | NETO1 |
| Q8TDL5 | BPI fold-containing family B member 1 | BPIFB1 |
| Q8TE56 | A disintegrin and metalloproteinase with | ADAMTS17 |
| | thrombospondin motifs 17 | |
| Q8TE57 | A disintegrin and metalloproteinase with | ADAMTS16 |
| | thrombospondin motifs 16 | |
| Q8TE58 | A disintegrin and metalloproteinase with | ADAMTS15 |
| | thrombospondin motifs 15 | |
| Q8TE59 | A disintegrin and metalloproteinase with | ADAMTS19 |
| | thrombospondin motifs 19 | |
| Q8TE60 | A disintegrin and metalloproteinase with | ADAMTS18 |
| | thrombospondin motifs 18 | |
| Q8TE99 | Acid phosphatase-like protein 2 | ACPL2 |
| Q8TER0 | Sushi, nidogen and EGF-like domain-containing | SNED1 |
| | protein 1 | |
| Q8TEU8 | WAP, kazal, immunoglobulin, kunitz and NTR | WFIKKN2 |
| | domain-containing protein 2 | |
| Q8WTQ1 | Beta-defensin 104 | DEFB104B |
| Q8WTR8 | Netrin-5 | NTN5 |
| Q8WTU2 | Scavenger receptor cysteine-rich domain- | SRCRB4D |
| | containing group B protein | |
| Q8WU66 | Protein TSPEAR | TSPEAR |
| Q8WUA8 | Tsukushin | TSKU |
| Q8WUF8 | Protein FAM172A | FAM172A |
| Q8WUJ1 | Neuferricin | CYB5D2 |
| Q8WUY1 | UPF0670 protein THEM6 | THEM6 |
| Q8WVN6 | Secreted and transmembrane protein 1 | SECTM1 |
| Q8WVQ1 | Soluble calcium-activated nucleotidase 1 | CANT1 |
| Q8WWA0 | Intelectin-1 | ITLN1 |
| Q8WWG1 | Neuregulin-4 | NRG4 |
| Q8WWQ2 | Inactive heparanase-2 | HPSE2 |
| Q8WWU7 | Intelectin-2 | ITLN2 |
| Q8WWY7 | WAP four-disulfide core domain protein 12 | WFDC12 |
| Q8WWY8 | Lipase member H | LIPH |
| Q8WWZ8 | Oncoprotein-induced transcript 3 protein | OIT3 |
| Q8WX39 | Epididymal-specific lipocalin-9 | LCN9 |
| Q8WXA2 | Prostate and testis expressed protein 1 | PATE1 |
| Q8WXD2 | Secretogranin-3 | SCG3 |
| Q8WXF3 | Relaxin-3 A chain | RLN3 |
| Q8WXI7 | Mucin-16 | MUC16 |
| Q8WXQ8 | Carboxypeptidase A5 | CPA5 |
| Q8WXS8 | A disintegrin and metalloproteinase with | ADAMTS14 |
| | thrombospondin motifs 14 | |
| Q92484 | Acid sphingomyelinase-like phosphodiesterase 3a | SMPDL3A |
| Q92485 | Acid sphingomyelinase-like phosphodiesterase 3b | SMPDL3B |
| Q92496 | Complement factor H-related protein 4 | CFHR4 |
| Q92520 | Protein FAM3C | FAM3C |
| Q92563 | Testican-2 | SPOCK2 |
| Q92583 | C-C motif chemokine 17 | CCL17 |
| Q92626 | Peroxidasin homolog | PXDN |
| Q92743 | Serine protease HTRA1 | HTRA1 |
| Q92752 | Tenascin-R | TNR |
| Q92765 | Secreted frizzled-related protein 3 | FRZB |
| Q92819 | Hyaluronan synthase 2 | HAS2 |
| Q92820 | Gamma-glutamyl hydrolase | GGH |
| Q92824 | Proprotein convertase subtilisin/kexin type 5 | PCSK5 |
| Q92832 | Protein kinase C-binding protein NELL1 | NELL1 |
| Q92838 | Ectodysplasin-A, membrane form | EDA |
| Q92874 | Deoxyribonuclease-1-like 2 | DNASE1L2 |
| Q92876 | Kallikrein-6 | KLK6 |
| Q92913 | Fibroblast growth factor 13 | FGF13 |
| Q92954 | Proteoglycan 4 C-terminal part | PRG4 |
| Q93038 | Tumor necrosis factor receptor superfamily | TNFRSF25 |
| | member 25 | |
| Q93091 | Ribonuclease K6 | RNASE6 |
| Q93097 | Protein Wnt-2b | WNT2B |
| Q93098 | Protein Wnt-8b | WNT8B |
| Q95460 | Major histocompatibility complex class I-related | MR1 |
| | gene protein | |
| Q969D9 | Thymic stromal lymphopoietin | TSLP |
| Q969E1 | Liver-expressed antimicrobial peptide 2 | LEAP2 |
| Q969H8 | UPF0556 protein C19orf10 | C19orf10 |
| Q969Y0 | NXPE family member 3 | NXPE3 |
| Q96A54 | Adiponectin receptor protein 1 | ADIPOR1 |
| Q96A83 | Collagen alpha-1(XXVI) chain | EMID2 |
| Q96A84 | EMI domain-containing protein 1 | EMID1 |
| Q96A98 | Tuberoinfundibular peptide of 39 residues | PTH2 |
| Q96A99 | Pentraxin-4 | PTX4 |
| Q96BH3 | Epididymal sperm-binding protein 1 | ELSPBP1 |
| Q96BQ1 | Protein FAM3D | FAM3D |
| Q96CG8 | Collagen triple helix repeat-containing protein 1 | CTHRC1 |
| Q96DA0 | Zymogen granule protein 16 homolog B | ZG16B |
| Q96DN2 | von Willebrand factor C and EGF domain- | VWCE |
| | containing protein | |
| Q96DR5 | BPI fold-containing family A member 2 | BPIFA2 |
| Q96DR8 | Mucin-like protein 1 | MUCL1 |
| Q96DX4 | RING finger and SPRY domain-containing protein 1 | RSPRY1 |
| Q96EE4 | Coiled-coil domain-containing protein 126 | CCDC126 |
| Q96GS6 | Abhydrolase domain-containing protein | FAM108A1 |
| | FAM108A1 | |
| Q96GW7 | Brevican core protein | BCAN |
| Q96HF1 | Secreted frizzled-related protein 2 | SFRP2 |
| Q96I82 | Kazal-type serine protease inhibitor domain- | KAZALD1 |
| | containing protein 1 | |
| Q96ID5 | Immunoglobulin superfamily member 21 | IGSF21 |
| Q96II8 | Leucine-rich repeat and calponin homology | LRCH3 |
| | domain-containing protein 3 | |
| Q96IY4 | Carboxypeptidase B2 | CPB2 |
| Q96JB6 | Lysyl oxidase homolog 4 | LOXL4 |
| Q96JK4 | HHIP-like protein 1 | HHIPL1 |
| Q96KN2 | Beta-Ala-His dipeptidase | CNDP1 |
| Q96KW9 | Protein SPACA7 | SPACA7 |
| Q96KX0 | Lysozyme-like protein 4 | LYZL4 |
| Q96L15 | Ecto-ADP-ribosyltransferase 5 | ART5 |
| Q96LB8 | Peptidoglycan recognition protein 4 | PGLYRP4 |
| Q96LB9 | Peptidoglycan recognition protein 3 | PGLYRP3 |
| Q96LC7 | Sialic acid-binding Ig-like lectin 10 | SIGLEC10 |
| Q96LR4 | Protein FAM19A4 | FAM19A4 |
| Q96MK3 | Protein FAM20A | FAM20A |
| Q96MS3 | Glycosyltransferase 1 domain-containing protein 1 | GLT1D1 |
| Q96NY8 | Processed poliovirus receptor-related protein 4 | PVRL4 |
| Q96NZ8 | WAP, kazal, immunoglobulin, kunitz and NTR | WFIKKN1 |
| | domain-containing protein 1 | |
| Q96NZ9 | Proline-rich acidic protein 1 | PRAP1 |
| Q96P44 | Collagen alpha-1(XXI) chain | COL21A1 |
| Q96PB7 | Noelin-3 | OLFM3 |
| Q96PC5 | Melanoma inhibitory activity protein 2 | MIA2 |
| Q96PD5 | N-acetylmuramoyl-L-alanine amidase | PGLYRP2 |
| Q96PH6 | Beta-defensin 118 | DEFB118 |
| Q96PL1 | Secretoglobin family 3A member 2 | SCGB3A2 |
| Q96PL2 | Beta-tectorin | TECTB |
| Q96QH8 | Sperm acrosome-associated protein 5 | SPACA5 |
| Q96QR1 | Secretoglobin family 3A member 1 | SCGB3A1 |
| Q96QU1 | Protocadherin-15 | PCDH15 |
| Q96QV1 | Hedgehog-interacting protein | HHIP |
| Q96RW7 | Hemicentin-1 | HMCN1 |
| Q96S42 | Nodal homolog | NODAL |
| Q96S86 | Hyaluronan and proteoglycan link protein 3 | HAPLN3 |
| Q96SL4 | Glutathione peroxidase 7 | GPX7 |
| Q96SM3 | Probable carboxypeptidase X1 | CPXM1 |
| Q96T91 | Glycoprotein hormone alpha-2 | GPHA2 |
| Q99062 | Granulocyte colony-stimulating factor receptor | CSF3R |
| Q99102 | Mucin-4 alpha chain | MUC4 |
| Q99217 | Amelogenin, X isoform | AMELX |
| Q99218 | Amelogenin, Y isoform | AMELY |
| Q99435 | Protein kinase C-binding protein NELL2 | NELL2 |
| Q99470 | Stromal cell-derived factor 2 | SDF2 |
| Q99542 | Matrix metalloproteinase-19 | MMP19 |
| Q99574 | Neuroserpin | SERPINI1 |
| Q99584 | Protein S100-A13 | S100A13 |
| Q99616 | C-C motif chemokine 13 | CCL13 |
| Q99645 | Epiphycan | EPYC |
| Q99674 | Cell growth regulator with EF hand domain | CGREF1 |
| | protein 1 | |
| Q99715 | Collagen alpha-1(XII) chain | COL12A1 |
| Q99727 | Metalloproteinase inhibitor 4 | TIMP4 |
| Q99731 | C-C motif chemokine 19 | CCL19 |
| Q99748 | Neurturin | NRTN |
| Q99935 | Proline-rich protein 1 | PROL1 |
| Q99942 | E3 ubiquitin-protein ligase RNF5 | RNF5 |
| Q99944 | Epidermal growth factor-like protein 8 | EGFL8 |
| Q99954 | Submaxillary gland androgen-regulated protein 3A | SMR3A |
| Q99969 | Retinoic acid receptor responder protein 2 | RARRES2 |
| Q99972 | Myocilin | MYOC |
| Q99983 | Osteomodulin | OMD |
| Q99985 | Semaphorin-3C | SEMA3C |
| Q99988 | Growth/differentiation factor 15 | GDF15 |
| Q9BPW4 | Apolipoprotein L4 | APOL4 |
| Q9BQ08 | Resistin-like beta | RETNLB |
| Q9BQ16 | Testican-3 | SPOCK3 |
| Q9BQ51 | Programmed cell death 1 ligand 2 | PDCD1LG2 |
| Q9BQB4 | Sclerostin | SOST |
| Q9BQI4 | Coiled-coil domain-containing protein 3 | CCDC3 |
| Q9BQP9 | BPI fold-containing family A member 3 | BPIFA3 |
| Q9BQR3 | Serine protease 27 | PRSS27 |
| Q9BQY6 | WAP four-disulfide core domain protein 6 | WFDC6 |
| Q9BRR6 | ADP-dependent glucokinase | ADPGK |
| Q9BS86 | Zona pellucida-binding protein 1 | ZPBP |
| Q9BSG0 | Protease-associated domain-containing protein 1 | PRADC1 |
| Q9BSG5 | Retbindin | RTBDN |
| Q9BT30 | Probable alpha-ketoglutarate-dependent | ALKBH7 |
| | dioxygenase ABH7 | |
| Q9BT56 | Spexin | C12orf39 |
| Q9BT67 | NEDD4 family-interacting protein 1 | NDFIP1 |
| Q9BTY2 | Plasma alpha-L-fucosidase | FUCA2 |
| Q9BU40 | Chordin-like protein 1 | CHRDL1 |
| Q9BUD6 | Spondin-2 | SPON2 |
| Q9BUN1 | Protein MENT | MENT |
| Q9BUR5 | Apolipoprotein O | APOO |
| Q9BV94 | ER degradation-enhancing alpha-mannosidase-like 2 | EDEM2 |
| Q9BWP8 | Collectin-11 | COLEC11 |
| Q9BWS9 | Chitinase domain-containing protein 1 | CHID1 |
| Q9BX67 | Junctional adhesion molecule C | JAM3 |
| Q9BX93 | Group XIIB secretory phospholipase A2-like | PLA2G12B |
| | protein | |
| Q9BXI9 | Complement C1q tumor necrosis factor-related | C1QTNF6 |
| | protein 6 | |
| Q9BXJ0 | Complement C1q tumor necrosis factor-related | C1QTNF5 |
| | protein 5 | |
| Q9BXJ1 | Complement C1q tumor necrosis factor-related | C1QTNF1 |
| | protein 1 | |
| Q9BXJ2 | Complement C1q tumor necrosis factor-related | C1QTNF7 |
| | protein 7 | |
| Q9BXJ3 | Complement C1q tumor necrosis factor-related | C1QTNF4 |
| | protein 4 | |
| Q9BXJ4 | Complement C1q tumor necrosis factor-related | C1QTNF3 |
| | protein 3 | |
| Q9BXJ5 | Complement C1q tumor necrosis factor-related | C1QTNF2 |
| | protein 2 | |
| Q9BXN1 | Asporin | ASPN |
| Q9BXP8 | Pappalysin-2 | PAPPA2 |
| Q9BXR6 | Complement factor H-related protein 5 | CFHR5 |
| Q9BXS0 | Collagen alpha-1(XXV) chain | COL25A1 |
| Q9BXX0 | EMILIN-2 | EMILIN2 |
| Q9BXY4 | R-spondin-3 | RSPO3 |
| Q9BY15 | EGF-like module-containing mucin-like hormone | EMR3 |
| | receptor-like 3 subunit beta | |
| Q9BY50 | Signal peptidase complex catalytic subunit | SEC11C |
| | SEC11C | |
| Q9BY76 | Angiopoietin-related protein 4 | ANGPTL4 |
| Q9BYF1 | Processed angiotensin-converting enzyme 2 | ACE2 |
| Q9BYJ0 | Fibroblast growth factor-binding protein 2 | FGFBP2 |
| Q9BYW3 | Beta-defensin 126 | DEFB126 |
| Q9BYX4 | Interferon-induced helicase C domain-containing | IFIH1 |
| | protein 1 | |
| Q9BYZ8 | Regenerating islet-derived protein 4 | REG4 |
| Q9BZ76 | Contactin-associated protein-like 3 | CNTNAP3 |
| Q9BZG9 | Ly-6/neurotoxin-like protein 1 | LYNX1 |
| Q9BZJ3 | Tryptase delta | TPSD1 |
| Q9BZM1 | Group XIIA secretory phospholipase A2 | PLA2G12A |
| Q9BZM2 | Group IIF secretory phospholipase A2 | PLA2G2F |
| Q9BZM5 | NKG2D ligand 2 | ULBP2 |
| Q9BZP6 | Acidic mammalian chitinase | CHIA |
| Q9BZZ2 | Sialoadhesin | SIGLEC1 |
| Q9C0B6 | Protein FAM5B | FAM5B |
| Q9GZM7 | Tubulointerstitial nephritis antigen-like | TINAGL1 |
| Q9GZN4 | Brain-specific serine protease 4 | PRSS22 |
| Q9GZP0 | Platelet-derived growth factor D, receptor- | PDGFD |
| | binding form | |
| Q9GZT5 | Protein Wnt-10a | WNT10A |
| Q9GZU5 | Nyctalopin | NYX |
| Q9GZV7 | Hyaluronan and proteoglycan link protein 2 | HAPLN2 |
| Q9GZV9 | Fibroblast growth factor 23 | FGF23 |
| Q9GZX9 | Twisted gastrulation protein homolog 1 | TWSG1 |
| Q9GZZ7 | GDNF family receptor alpha-4 | GFRA4 |
| Q9GZZ8 | Extracellular glycoprotein lacritin | LACRT |
| Q9H0B8 | Cysteine-rich secretory protein LCCL domain- | CRISPLD2 |
| | containing 2 | |
| Q9H106 | Signal-regulatory protein delta | SIRPD |
| Q9H114 | Cystatin-like 1 | CSTL1 |
| Q9H173 | Nucleotide exchange factor SIL1 | SIL1 |
| Q9H1E1 | Ribonuclease 7 | RNASE7 |
| Q9H1F0 | WAP four-disulfide core domain protein 10A | WFDC10A |
| Q9H1J5 | Protein Wnt-8a | WNT8A |
| Q9H1J7 | Protein Wnt-5b | WNT5B |
| Q9H1M3 | Beta-defensin 129 | DEFB129 |
| Q9H1M4 | Beta-defensin 127 | DEFB127 |
| Q9H1Z8 | Augurin | C2orf40 |
| Q9H239 | Matrix metalloproteinase-28 | MMP28 |
| Q9H2A7 | C-X-C motif chemokine 16 | CXCL16 |
| Q9H2A9 | Carbohydrate sulfotransferase 8 | CHST8 |
| Q9H2R5 | Kallikrein-15 | KLK15 |
| Q9H2X0 | Chordin | CHRD |
| Q9H2X3 | C-type lectin domain family 4 member M | CLEC4M |
| Q9H306 | Matrix metalloproteinase-27 | MMP27 |
| Q9H324 | A disintegrin and metalloproteinase with | ADAMTS10 |
| | thrombospondin motifs 10 | |
| Q9H336 | Cysteine-rich secretory protein LCCL domain- | CRISPLD1 |
| | containing 1 | |
| Q9H3E2 | Sorting nexin-25 | SNX25 |
| Q9H3R2 | Mucin-13 | MUC13 |
| Q9H3U7 | SPARC-related modular calcium-binding protein 2 | SMOC2 |
| Q9H3Y0 | Peptidase inhibitor R3HDML | R3HDML |
| Q9H4A4 | Aminopeptidase B | RNPEP |
| Q9H4F8 | SPARC-related modular calcium-binding protein 1 | SMOC1 |
| Q9H4G1 | Cystatin-9-like | CST9L |
| Q9H5V8 | CUB domain-containing protein 1 | CDCP1 |
| Q9H6B9 | Epoxide hydrolase 3 | EPHX3 |
| Q9H6E4 | Coiled-coil domain-containing protein 134 | CCDC134 |
| Q9H741 | UPF0454 protein C12orf49 | C12orf49 |
| Q9H772 | Gremlin-2 | GREM2 |
| Q9H7Y0 | Deleted in autism-related protein 1 | CXorf36 |
| Q9H8L6 | Multimerin-2 | MMRN2 |
| Q9H9S5 | Fukutin-related protein | FKRP |
| Q9HAT2 | Sialate O-acetylesterase | SIAE |
| Q9HB40 | Retinoid-inducible serine carboxypeptidase | SCPEP1 |
| Q9HB63 | Netrin-4 | NTN4 |
| Q9HBJ0 | Placenta-specific protein 1 | PLAC1 |
| Q9HC23 | Prokineticin-2 | PROK2 |
| Q9HC57 | WAP four-disulfide core domain protein 1 | WFDC1 |
| Q9HC73 | Cytokine receptor-like factor 2 | CRLF2 |
| Q9HC84 | Mucin-5B | MUC5B |
| Q9HCB6 | Spondin-1 | SPON1 |
| Q9HCQ7 | Neuropeptide NPSF | NPVF |
| Q9HCT0 | Fibroblast growth factor 22 | FGF22 |
| Q9HD89 | Resistin | RETN |
| Q9NNX1 | Tuftelin | TUFT1 |
| Q9NNX6 | CD209 antigen | CD209 |
| Q9NP55 | BPI fold-containing family A member 1 | BPIFA1 |
| Q9NP70 | Ameloblastin | AMBN |
| Q9NP95 | Fibroblast growth factor 20 | FGF20 |
| Q9NP99 | Triggering receptor expressed on myeloid cells 1 | TREM1 |
| Q9NPA2 | Matrix metalloproteinase-25 | MMP25 |
| Q9NPE2 | Neugrin | NGRN |
| Q9NPH0 | Lysophosphatidic acid phosphatase type 6 | ACP6 |
| Q9NPH6 | Odorant-binding protein 2b | OBP2B |
| Q9NQ30 | Endothelial cell-specific molecule 1 | ESM1 |
| Q9NQ36 | Signal peptide, CUB and EGF-like domain- | SCUBE2 |
| | containing protein 2 | |
| Q9NQ38 | Serine protease inhibitor Kazal-type 5 | SPINK5 |
| Q9NQ76 | Matrix extracellular phosphoglycoprotein | MEPE |
| Q9NQ79 | Cartilage acidic protein 1 | CRTAC1 |
| Q9NR16 | Scavenger receptor cysteine-rich type 1 | CD163L1 |
| | protein M160 | |
| Q9NR23 | Growth/differentiation factor 3 | GDF3 |
| Q9NR71 | Neutral ceramidase | ASAH2 |
| Q9NR99 | Matrix-remodeling-associated protein 5 | MXRA5 |
| Q9NRA1 | Platelet-derived growth factor C | PDGFC |
| Q9NRC9 | Otoraplin | OTOR |
| Q9NRE1 | Matrix metalloproteinase-26 | MMP26 |
| Q9NRJ3 | C-C motif chemokine 28 | CCL28 |
| Q9NRM1 | Enamelin | ENAM |
| Q9NRN5 | Olfactomedin-like protein 3 | OLFML3 |
| Q9NRR1 | Cytokine-like protein 1 | CYTL1 |
| Q9NS15 | Latent-transforming growth factor beta-binding | LTBP3 |
| | protein 3 | |
| Q9NS62 | Thrombospondin type-1 domain-containing | THSD1 |
| | protein 1 | |
| Q9NS71 | Gastrokine-1 | GKN1 |
| Q9NS98 | Semaphorin-3G | SEMA3G |
| Q9NSA1 | Fibroblast growth factor 21 | FGF21 |
| Q9NT22 | EMILIN-3 | EMILIN3 |
| Q9NTU7 | Cerebellin-4 | CBLN4 |
| Q9NVR0 | Kelch-like protein 11 | KLHL11 |
| Q9NWH7 | Spermatogenesis-associated protein 6 | SPATA6 |
| Q9NXC2 | Glucose-fructose oxidoreductase domain- | GFOD1 |
| | containing protein 1 | |
| Q9NY56 | Odorant-binding protein 2a | OBP2A |
| Q9NY84 | Vascular non-inflammatory molecule 3 | VNN3 |
| Q9NZ20 | Group 3 secretory phospholipase A2 | PLA2G3 |
| Q9NZC2 | Triggering receptor expressed on myeloid cells 2 | TREM2 |
| Q9NZK5 | Adenosine deaminase CECR1 | CECR1 |
| Q9NZK7 | Group IIE secretory phospholipase A2 | PLA2G2E |
| Q9NZP8 | Complement C1r subcomponent-like protein | C1RL |
| Q9NZV1 | Cysteine-rich motor neuron 1 protein | CRIM1 |
| Q9NZW4 | Dentin sialoprotein | DSPP |
| Q9P0G3 | Kallikrein-14 | KLK14 |
| Q9P0W0 | Interferon kappa | IFNK |
| Q9P218 | Collagen alpha-1(XX) chain | COL20A1 |
| Q9P2C4 | Transmembrane protein 181 | TMEM181 |
| Q9P2K2 | Thioredoxin domain-containing protein 16 | TXNDC16 |
| Q9P2N4 | A disintegrin and metalloproteinase with | ADAMTS9 |
| | thrombospondin motifs 9 | |
| Q9UBC7 | Galanin-like peptide | GALP |
| Q9UBD3 | Cytokine SCM-1 beta | XCL2 |
| Q9UBD9 | Cardiotrophin-like cytokine factor 1 | CLCF1 |
| Q9UBM4 | Opticin | OPTC |
| Q9UBP4 | Dickkopf-related protein 3 | DKK3 |
| Q9UBQ6 | Exostosin-like 2 | EXTL2 |
| Q9UBR5 | Chemokine-like factor | CKLF |
| Q9UBS5 | Gamma-aminobutyric acid type B receptor subunit 1 | GABBR1 |
| Q9UBT3 | Dickkopf-related protein 4 short form | DKK4 |
| Q9UBU2 | Dickkopf-related protein 2 | DKK2 |
| Q9UBU3 | Ghrelin-28 | GHRL |
| Q9UBV4 | Protein Wnt-16 | WNT16 |
| Q9UBX5 | Fibulin-5 | FBLN5 |
| Q9UBX7 | Kallikrein-11 | KLK11 |
| Q9UEF7 | Klotho | KL |
| Q9UFP1 | Protein FAM198A | FAM198A |
| Q9UGM3 | Deleted in malignant brain tumors 1 protein | DMBT1 |
| Q9UGM5 | Fetuin-B | FETUB |
| Q9UGP8 | Translocation protein SEC63 homolog | SEC63 |
| Q9UHF0 | Neurokinin-B | TAC3 |
| Q9UHF1 | Epidermal growth factor-like protein 7 | EGFL7 |
| Q9UHG2 | ProSAAS | PCSK1N |
| Q9UHI8 | A disintegrin and metalloproteinase with | ADAMTS1 |
| | thrombospondin motifs 1 | |
| Q9UHL4 | Dipeptidyl peptidase 2 | DPP7 |
| Q9UI42 | Carboxypeptidase A4 | CPA4 |
| Q9UIG4 | Psoriasis susceptibility 1 candidate gene 2 protein | PSORS1C2 |
| Q9UIK5 | Tomoregulin-2 | TMEFF2 |
| Q9UIQ6 | Leucyl-cystinyl aminopeptidase, pregnancy serum | LNPEP |
| | form | |
| Q9UJA9 | Ectonucleotide pyrophosphatase/phosphodiesterase | ENPP5 |
| | family member 5 | |
| Q9UJH8 | Meteorin | METRN |
| Q9UJJ9 | N-acetylglucosamine-1-phosphotransferase | GNPTG |
| | subunit gamma | |
| Q9UJW2 | Tubulointerstitial nephritis antigen | TINAG |
| Q9UK05 | Growth/differentiation factor 2 | GDF2 |
| Q9UK55 | Protein Z-dependent protease inhibitor | SERPINA10 |
| Q9UK85 | Dickkopf-like protein 1 | DKKL1 |
| Q9UKJ1 | Paired immunoglobulin-like type 2 receptor alpha | PILRA |
| Q9UKP4 | A disintegrin and metalloproteinase with | ADAMTS7 |
| | thrombospondin motifs 7 | |
| Q9UKP5 | A disintegrin and metalloproteinase with | ADAMTS6 |
| | thrombospondin motifs 6 | |
| Q9UKQ2 | Disintegrin and metalloproteinase domain- | ADAM28 |
| | containing protein 28 | |
| Q9UKQ9 | Kallikrein-9 | KLK9 |
| Q9UKR0 | Kallikrein-12 | KLK12 |
| Q9UKR3 | Kallikrein-13 | KLK13 |
| Q9UKU9 | Angiopoietin-related protein 2 | ANGPTL2 |
| Q9UKZ9 | Procollagen C-endopeptidase enhancer 2 | PCOLCE2 |
| Q9UL52 | Transmembrane protease serine 11E non- | TMPRSS11E |
| | catalytic chain | |
| Q9ULC0 | Endomucin | EMCN |
| Q9ULI3 | Protein HEG homolog 1 | HEG1 |
| Q9ULZ1 | Apelin-13 | APLN |
| Q9ULZ9 | Matrix metalloproteinase-17 | MMP17 |
| Q9UM21 | Alpha-1,3-mannosyl-glycoprotein 4-beta-N- | MGAT4A |
| | acetylglucosaminyltransferase A soluble form | |
| Q9UM22 | Mammalian ependymin-related protein 1 | EPDR1 |
| Q9UM73 | ALK tyrosine kinase receptor | ALK |
| Q9UMD9 | 97 kDa linear IgA disease antigen | COL17A1 |
| Q9UMX5 | Neudesin | NENF |
| Q9UN73 | Protocadherin alpha-6 | PCDHA6 |
| Q9UNA0 | A disintegrin and metalloproteinase with | ADAMTS5 |
| | thrombospondin motifs 5 | |
| Q9UNI1 | Chymotrypsin-like elastase family member 1 | CELA1 |
| Q9UNK4 | Group IID secretory phospholipase A2 | PLA2G2D |
| Q9UP79 | A disintegrin and metalloproteinase with | ADAMTS8 |
| | thrombospondin motifs 8 | |
| Q9UPZ6 | Thrombospondin type-1 domain-containing | THSD7A |
| | protein 7A | |
| Q9UQ72 | Pregnancy-specific beta-1-glycoprotein 11 | PSG11 |
| Q9UQ74 | Pregnancy-specific beta-1-glycoprotein 8 | PSG8 |
| Q9UQC9 | Calcium-activated chloride channel regulator 2 | CLCA2 |
| Q9UQE7 | Structural maintenance of chromosomes protein 3 | SMC3 |
| Q9UQP3 | Tenascin-N | TNN |
| Q9Y223 | UDP-N-acetylglucosamine 2-epimerase | GNE |
| Q9Y240 | C-type lectin domain family 11 member A | CLEC11A |
| Q9Y251 | Heparanase 8 kDa subunit | HPSE |
| Q9Y258 | C-C motif chemokine 26 | CCL26 |
| Q9Y264 | Angiopoietin-4 | ANGPT4 |
| Q9Y275 | Tumor necrosis factor ligand superfamily member | TNFSF13B |
| | 13b, membrane form | |
| Q9Y287 | BRI2 intracellular domain | ITM2B |
| Q9Y2E5 | Epididymis-specific alpha-mannosidase | MAN2B2 |
| Q9Y334 | von Willebrand factor A domain-containing | VWA7 |
| | protein 7 | |
| Q9Y337 | Kallikrein-5 | KLK5 |
| Q9Y3B3 | Transmembrane emp24 domain-containing protein 7 | TMED7 |
| Q9Y3E2 | BolA-like protein 1 | BOLA1 |
| Q9Y426 | C2 domain-containing protein 2 | C2CD2 |
| Q9Y4K0 | Lysyl oxidase homolog 2 | LOXL2 |
| Q9Y4X3 | C-C motif chemokine 27 | CCL27 |
| Q9Y5C1 | Angiopoietin-related protein 3 | ANGPTL3 |
| Q9Y5I2 | Protocadherin alpha-10 | PCDHA10 |
| Q9Y5I3 | Protocadherin alpha-1 | PCDHA1 |
| Q9Y5K2 | Kallikrein-4 | KLK4 |
| Q9Y5L2 | Hypoxia-inducible lipid droplet-associated protein | HILPDA |
| Q9Y5Q5 | Atrial natriuretic peptide-converting enzyme | CORIN |
| Q9Y5R2 | Matrix metalloproteinase-24 | MMP24 |
| Q9Y5U5 | Tumor necrosis factor receptor superfamily | TNFRSF18 |
| | member 18 | |
| Q9Y5W5 | Wnt inhibitory factor 1 | WIF1 |
| Q9Y5X9 | Endothelial lipase | LIPG |
| Q9Y625 | Secreted glypican-6 | GPC6 |
| Q9Y646 | Carboxypeptidase Q | CPQ |
| Q9Y6C2 | EMILIN-1 | EMILIN1 |
| Q9Y6F9 | Protein Wnt-6 | WNT6 |
| Q9Y6I9 | Testis-expressed sequence 264 protein | TEX264 |
| Q9Y6L7 | Tolloid-like protein 2 | TLL2 |
| Q9Y6N3 | Calcium-activated chloride channel regulator | CLCA3P |
| | family member 3 | |
| Q9Y6N6 | Laminin subunit gamma-3 | LAMC3 |
| Q9Y6R7 | IgGFc-binding protein | FCGBP |
| Q9Y6Y9 | Lymphocyte antigen 96 | LY96 |
| Q9Y6Z7 | Collectin-10 | COLEC10 |

In some embodiments, the compositions and methods of the invention provide for the delivery of one or more mRNAs encoding one or more additional exemplary proteins listed in Table 2; thus, compositions of the invention may comprise an mRNA encoding a protein listed in Table 2 (or a homolog thereof) along with other components set out herein, and methods of the invention may comprise preparing and/or administering a composition comprising an mRNA encoding a protein chosen from the proteins listed in Table 2 (or a homolog thereof) along with other components set out herein.

TABLE 2
Additional Exemplary Proteins

| Uniprot ID | Protein Name | Gene Name |
|---|---|---|
| A6NGW2 | Putative stereocilin-like protein | STRCP1 |
| A6NIE9 | Putative serine protease 29 | PRSS29P |
| A6NJ16 | Putative V-set and immunoglobulin | IGHV4OR15-8 |
| | domain-containing-like protein | |
| | IGHV4OR15-8 | |
| A6NJS3 | Putative V-set and immunoglobulin | IGHV1OR21-1 |
| | domain-containing-like protein | |
| | IGHV1OR21-1 | |
| A6NMY6 | Putative annexin A2-like protein | ANXA2P2 |
| A8MT79 | Putative zinc-alpha-2-glycoprotein-like 1 | |
| A8MWS1 | Putative killer cell immunoglobulin-like | KIR3DP1 |
| | receptor like protein KIR3DP1 | |
| A8MXU0 | Putative beta-defensin 108A | DEFB108P1 |
| C9JUS6 | Putative adrenomedullin-5-like protein | ADM5 |
| P0C7V7 | Putative signal peptidase complex | SEC11B |
| | catalytic subunit SEC11B | |
| P0C854 | Putative cat eye syndrome critical region | CECR9 |
| | protein 9 | |
| Q13046 | Putative pregnancy-specific beta-1- | PSG7 |
| | glycoprotein 7 | |
| Q16609 | Putative apolipoprotein(a)-like protein 2 | LPAL2 |
| Q2TV78 | Putative macrophage-stimulating protein | MST1P9 |
| | MSTP9 | |
| Q5JQD4 | Putative peptide YY-3 | PYY3 |
| Q5R387 | Putative inactive group IIC secretory | PLA2G2C |
| | phospholipase A2 | |
| Q5VSP4 | Putative lipocalin 1-like protein 1 | LCN1P1 |
| Q5W188 | Putative cystatin-9-like protein CST9LP1 | CST9LP1 |
| Q6UXR4 | Putative serpin A13 | SERPINA13P |
| Q86SH4 | Putative testis-specific prion protein | PRNT |
| Q86YQ2 | Putative latherin | LATH |
| Q8IVG9 | Putative humanin peptide | MT-RNR2 |
| Q8NHM4 | Putative trypsin-6 | TRY6 |
| Q8NHW4 | C-C motif chemokine 4-like | CCL4L2 |
| Q9H7L2 | Putative killer cell immunoglobulin-like | KIR3DX1 |
| | receptor-like protein KIR3DX1 | |
| Q9NRI6 | Putative peptide YY-2 | PYY2 |
| Q9UF72 | Putative TP73 antisense gene protein 1 | TP73-AS1 |
| Q9UKY3 | Putative inactive carboxylesterase 4 | CES1P1 |

The Uniprot IDs set forth in Table 1 and Table 2 refer to the human versions the listed proteins and the sequences of each are available from the Uniprot database. Sequences of the listed proteins are also generally available for various animals, including various mammals and animals of veterinary or industrial interest. Accordingly, in some embodiments, compositions and methods of the invention provide for the delivery of one or more mRNAs encoding one or more proteins chosen from mammalian homologs or homologs from an animal of veterinary or industrial interest of the secreted proteins listed in Table 1 and Table 2; thus, compositions of the invention may comprise an mRNA encoding a protein chosen from mammalian homologs or homologs from an animal of veterinary or industrial interest of a protein listed in Table 1 and Table 2 along with other components set out herein, and methods of the invention may comprise preparing and/or administering a composition comprising an mRNA encoding a protein chosen from mammalian homologs or homologs from an animal of veterinary or industrial interest of a protein listed in Table 1 and Table 2 along with other components set out herein. In some embodiments, mammalian homologs are chosen from mouse, rat, hamster, gerbil, horse, pig, cow, llama, alpaca, mink, dog, cat, ferret, sheep, goat, or camel homologs. In some embodiments, the animal of veterinary or industrial interest is chosen from the mammals listed above and/or chicken, duck, turkey, salmon, catfish, or tilapia.

In embodiments, the compositions and methods of the invention provide for the delivery of mRNA encoding a lysosomal protein chosen from Table 3. In some embodiments, the compositions and methods of the invention provide for the delivery of one or more mRNAs encoding one or more lysosomal and/or related proteins listed in Table 3; thus, compositions of the invention may comprise an mRNA encoding a protein listed in Table 3 (or a homolog thereof) along with other components set out herein, and methods of the invention may comprise preparing and/or administering a composition comprising an mRNA encoding a protein chosen from the proteins listed in Table 3 (or a homolog thereof) along with other components set out herein.

TABLE 3
Lysosomal and Related Proteins

α-fucosidase
α-galactosidase
α-glucosidase
α-Iduronidase
α-mannosidase
α-N-acetylgalactosaminidase (α-galactosidase B)
β-galactosidase
β-glucuronidase
β-hexosaminidase
β-mannosidase
3-hydroxy-3-methylglutaryl-CoA (HMG-CoA) lyase
3-methylcrotonyl-CoA carboxylase
3-O-sulfogalactosyl cerebroside sulfatase (arylsulfatase A)
acetyl-CoA transferase
acid alpha-glucosidase
acid ceramidase
acid lipase
acid phosphatase
acid sphingomyelinase
alpha-galactosidase A
arylsulfatase A
beta-galactosidase
beta-glucocerebrosidase
beta-hexosaminidase
biotinidase
cathepsin A
cathepsin K
CLN3
CLN5
CLN6
CLN8
CLN9
cystine transporter (cystinosin)
cytosolic protein beta3A subunit of the adaptor protein-3 complex, AP3
formyl-Glycine generating enzyme (FGE)
Galactocerebrosidase
galactose-1-phosphate uridyltransferase (GALT)
galactose 6-sulfate sulfatase
(also known as N-acetylgalactosamine-6-sulfatase)
Glucocerebrosidase
glucuronate sulfatase
glucuronidase
glycoprotein cleaving enzymes
glycosaminoglycan cleaving enzymes
glycosylasparaginase (aspartylglucosaminidase)
GM2-AP
Heparan-alpha-glucosaminide N-acetyltransferase (HGSNAT, TMEM76)
Heparan sulfatase
hexosaminidase A lysosomal proteases methylmalonyl-CoA mutase
Hyaluronidase
Iduronate sulfatase
LAMP-2
lysosomal α-mannosidase
Lysosomal p40 (C2orf18)
Major facilitator superfamily domain containing 8 protein
(MFSD8 or CLN7)
N-acetylgalactosamine 4-sulfatase
N-acetyl glucosamine 6-sulfatase
N-acetyl glucosaminidase
N-acetylglucosamine-1-phosphate transferase
NPC1
NPC2
palmitoyl-protein thioesterase
palmitoyl-protein thioesterase (CLN1)
Saposin A (Sphingolipid activator protein A)
Saposin B (Sphingolipid activator protein B)
Saposin C (Sphingolipid activator protein C)
Saposin D (Sphingolipid activator protein D)
sialic acid transporter (sialin)
Sialidase
Sialin
Sulfatase
Transmembrane protein 74 (TMEM74)
tripeptidyl-peptidase
tripeptidyl-peptidase I (CLN2)
UDP-N-acetylglucosamine- phosphotransferase

Information regarding lysosomal proteins is available from Lubke et al., “Proteomics of the Lysosome,” Biochim Biophys Acta. (2009) 1793: 625-635. In some embodiments, the protein listed in Table 3 and encoded by mRNA in the compositions and methods of the invention is a human protein. Sequences of the listed proteins are also available for various animals, including various mammals and animals of veterinary or industrial interest as described above.

In some embodiments, the compositions and methods of the invention provide for the delivery of mRNA encoding a therapeutic peptide, polypeptide or protein to a subject, wherein the subject suffers from disease or disorder that is due to a deficiency in the peptide, polypeptide or protein encoded by the mRNA in the subject. The deficiency may be due to non-expression of the peptide, polypeptide or protein; expression of a non-functional peptide, polypeptide or protein, a dysfunctional peptide, polypeptide or protein, or peptide, polypeptide or protein with reduced function; or other functional impediment to the peptide, polypeptide or proteins. Diseases or disorders of this nature are commonly referred to as “protein deficiencies”. Typically, these diseases or disorders are caused by one or more mutations in the gene encoding said peptide, polypeptide or protein in the subject. The replacement peptide, polypeptide or protein encoded by the mRNA does not include the one or more mutations that are the underlying cause of the protein deficiency. Diseases or disorders that are due to a protein deficiency include cystic fibrosis, lysosomal storage diseases, metabolic disorders (e.g., urea cycle disorders), etc.

In other embodiments, the compositions and methods of the invention provide for the delivery of mRNA encoding a therapeutic peptide, polypeptide or protein. Such therapeutic peptides, polypeptides or proteins include antibodies, immunogens, cytokines, allergens, etc.

In some embodiments, the compositions and methods of the invention provide for the delivery of mRNA encoding a therapeutic protein (e.g., cytosolic, transmembrane or secreted) such as those listed in Table 4. In some embodiments, the compositions and methods of the invention provide for the delivery of an mRNA encoding a therapeutic protein useful in treating a disease or disorder (i.e., indication) listed in Table 4; thus, compositions of the invention may comprise an mRNA encoding a therapeutic protein listed or not listed in Table 4 (or a homolog thereof, as discussed below) along with other components set out herein for treating a disease or disorder (i.e., indication) listed in Table 4, and methods of the invention may comprise preparing and/or administering a composition comprising an mRNA encoding a such a protein (or a homolog thereof, as discussed below) along with other components set out herein for treatment of a disease or disorder listed in Table 4.

TABLE 4
Exemplary Indications and Related Proteins

| Indication | Therapeutic Protein |
|---|---|
| 3-Methylcrotonyl-CoA carboxylase deficiency | Methylcrotonoyl-CoA carboxylase |
| 3-Methylglutaconic aciduria | Methylglutaconyl-CoA hydratase |
| Actinic keratosis | |
| Acute intermittent porphyria | Porphobilinogen deaminase |
| Acute lymphocytic leukemia | |
| Acute myeloid leukemia | |
| Addison's disease | |
| Adenosine deaminase deficiency | Adenosine deaminase |
| Adrenoleukodystrophy | ABCD1 |
| Adrenomyeloneuropathy | |
| AIDS/HIV | |
| Alcohol use disorders | |
| Alkaptonuria | Homogentisate 1,2-dioxygenase |
| Allergic asthma | Anti-IgE mAb |
| Allergies (dermatitis, rhinitis) | |
| Alopecia areata | |
| Alpers' disease | POLG |
| Alpers-Huttenlocher syndrome | |
| Alpha 1-antitrypsin deficiency | Alpha 1 protease inhibitor |
| Alpha-mannosidosis | Alpha-D-mannosidase |
| Alport syndrome | |
| Alzheimer's disease | |
| Amyloid light-chain amyloidosis | |
| Amyotrophic lateral sclerosis (ALS) | |
| Anemia | Erythropoietin |
| Aortic valve stenosis | |
| Argininemia | Arginase |
| Argininosuccinic acidemia | Argininosuccinate lyase |
| Arrhythmogenic right ventricular dysplasia | |
| Autism | |
| Autosomal dominant and recessive progressive | |
| external ophthalmoplegia with mitochondrial DNA | |
| deletions | |
| Autosomal recessive polycystic kidney disease | ARPKD |
| Bacterial infections | |
| Basal cell carcinoma | |
| Batten disease | Battenin + others |
| B-cell chronic lymphocytic leukemia | |
| Becker muscular dystrophy | Dystrophin |
| Beta-thalassemia | Beta globin |
| Binge eating disorder | |
| Bipolar disorder | |
| Bladder cancer | |
| Blepharospasm, Cervical dystonia, Chronic migraine, | Botulinum toxin |
| more | |
| Bronchiolitis obliterans | |
| Brugada syndrome | |
| Buerger's disease | |
| CACNA1A | |
| CACNB4-related Episodic Ataxia Type 2 | |
| Cancer and depression | |
| Cancer and sexual dysfunction | |
| Cancer in pregnancy | |
| Carbamylphosphate synthetase deficiency | Carbamylphosphate synthetase |
| Carcinoma of the gallbladder | |
| Cardiomyopathy (diabetic) | |
| Cardiomyopathy (hypertrophic) | |
| Carnitine uptake defect | SLC22A5 |
| Catecholaminergic polymorphic ventricular | |
| tachycardia | |
| CDKL5-related Atypical Rett Syndrome | |
| Celiac disease | |
| Cellulitis | |
| Cerebrovascular disease | |
| Cervix uteri cancer | |
| Chronic fatigue syndrome | |
| Chronic graft versus host disease | |
| Chronic idiopathic urticaria | |
| Chronic immune thrombocytopenia | Thrombopoietin |
| Chronic kidney kisease | |
| Chronic liver disease | |
| Chronic lymphocytic leukemia | |
| Chronic myeloid leukemia | |
| Chronic pancreatitis | |
| Cirrhosis of the liver | |
| Citrullinemia, type I | Argininosuccinate synthase |
| Classic Rett Syndrome | |
| Classical galactosemia | Galactose-1-phosphate uridylyltransferase |
| Clostridium difficile associated diarrhea | |
| Clotting disorders | |
| COAD/COPD | |
| Cocaine addiction | |
| COL4A5-related disorders | |
| Cold contact urticaria | |
| Contraception, female | |
| Coronary artery diseases | |
| Corpus uteri cancer | |
| Corticobasal degeneration | |
| Crigler-Najjar syndrome | UDP-glucuronosyltransferase |
| Critical limb ischemia | |
| CTNS-related cystinosis | |
| Cutaneous lupus erythematosus | |
| Cutaneous neuroendocrine carcinoma (Merkel Cell) | |
| Cystic fibrosis | CFTR |
| Cystic fibrosis | Deoxyribonuclease I |
| Cystinosis | Cystinosin |
| Cystinuria | SLC7A9 |
| Dementia (Lewy body) | |
| Depression | |
| Diabetic foot infections | |
| Diabetic foot ulcer | |
| Diabetic peripheral neuropathy | |
| Diabetic ulcers | |
| Diarrhoeal diseases | |
| Diffuse large B-cell lymphoma | |
| DiGeorge syndrome | |
| Diverticulitis | |
| Drug use disorders | |
| Duchenne muscular dystrophy | Dystrophin |
| Dysarthria | |
| Dyskinesia (levodopa-induced) | |
| Early-onset autosomal dominant Alzheimer's disease | |
| Eczema | |
| Ehlers-Danlos syndrome, type 1 | |
| EIF2B1 | |
| EIF2B2 | |
| EIF2B3 | |
| EIF2B4 | |
| EIF2B5-related childhood ataxia with central nervous | |
| system hypomyelination/vanishing white matter | |
| Eosinophilic esophagitis | |
| Epilepsy | |
| Erectile dysfunction | |
| Erythropoietic protoporphyria | Ferrochelatase |
| Esophageal carcinoma | |
| Essential tremor | |
| Fabry disease | Alpha galactosidase |
| Familial adenomatous polyposis | APC |
| Familial chylomicronemia | Lipoprotein lipase |
| Familial dysbetalipoproteinemia | Apolipoprotein E |
| Familial isolated dilated cardiomyopathy | |
| Familial mediterranean fever | Pyrin (MEFV) |
| Familial melanoma | |
| Female infertility | Follicle stimulating hormone |
| Female sexual dysfunction | |
| Fibromyalgia | |
| FMR1-related disorders | |
| Fracture healing | |
| Fragile X Premature Ovarian Failure Syndrome | |
| Fragile X syndrome | FMRP |
| Fragile X-Associated Tremor/Ataxia Syndrome | |
| Friedreich's ataxia | |
| Frontotemporal dementia | |
| Fryns syndrome | |
| Galactocerebrosidase deficiencies | |
| GALE deficiency | Galactose epimerase |
| GALK deficiency | Galactokinase |
| GALT-related galactosemia | |
| Gastric cancer | |
| Gastroesophageal reflux disease | |
| Gaucher disease | Glucocerebrosidase |
| Gilbert syndrome | UDP-glucuronosyltransferase |
| Glioblastoma multiforme | |
| Glomerulonephritis | |
| Glutaric acidemia, type I | Glutaryl-CoA dehydrogenase |
| GM2 gangliosidosis | HEXA, HEXB |
| Gout | Urate oxidase |
| Graft versus host disease | |
| Growth hormone deficiency | Growth hormone 1/Growth hormone 2 |
| Head and neck cancer, Metastatic colorectal cancer | Anti-EGFr mAb |
| Hearing loss, adult onset | |
| Heart failure | |
| Hemachromatosis | HFE protein |
| Hemifacial spasm | |
| Hemolytic uremic syndrome | Anti-complement factor C5 mAb |
| Hemophilia A | Factor VIII |
| Hemophilia A, Hemophilia B | Factor VII |
| Hemophilia B | Factor IX |
| Hepatitis B, Hepatitis C | Interferon alpha |
| HER2+ breast cancer, gastric cancer | Anti-HER2 mAb |
| Hereditary angioedema | C1 esterase inhibitor |
| Hereditary hemorrhagic telangiectasia | |
| Hereditary hemorrhagic telangiectasia (AT) | |
| Hereditary spherocytosis | |
| Hidradenitis suppurativa | |
| Homocystinuria | Cystathionine beta-synthase |
| Homozygous familial hypercholesterolemia | LDL receptor |
| Hunter syndrome (MPS II) | Iduronate-2-sulfatase |
| Huntington disease | Huntingtin |
| Hurler syndrome (MPS I) | Alpha-L iduronidase |
| Hydrolethalus | |
| Hyperalgesia | |
| Hyperbilirubinemia | |
| Hyperhidrosis | |
| Hyperlipidemia | |
| Hypermethioninemia | Methionine adenosyltransferase |
| Hyperoxaluria, type I | Serine-pyruvate aminotransferase |
| Hypertension | |
| Hyperuricemia | |
| Hyponatremia | |
| Hypoparathyroidism | Parathyroid hormone |
| Hypophosphatasia | TNSALP |
| Idiopathic pulmonary fibrosis | |
| Iminoglycinuria | |
| Immunoglobulin deficiency | Immunoglobulin |
| Infection (adenovirus) | |
| Infection (anthrax prophylaxis) | |
| Infection (BK virus) | |
| Infection (Clostridium difficile prophylaxis) | |
| Infection (Dengue fever prophylaxis) | |
| Infection (Epstein-Barr virus) | |
| Infection (Hepatitis-D) | |
| Infection (Lyme disease prophylaxis) | |
| Infection (Smallpox virus) | |
| Infectious diseases vaccines | Infectious antigen |
| Inflammatory heart diseases | |
| Insomnia | |
| Interstitial cystitis | |
| Iron-deficiency anaemia | |
| Irritable bowel disease | |
| Ischaemic heart disease | |
| Isovaleric aciduria | Isovaleric acid CoA dehydrogenase deficiency |
| Jansky-Bielschowsky disease | |
| Juvenile Batten disease | |
| Juvenile Neuronal Ceroid Lipofuscinosis (JNCL) | |
| Juvenile rheumatoid arthritis | TNF-alpha inhibitors |
| Kennedy's disease (SBMA) | |
| Keratoconus | |
| Krabbe disease | Galactocerebrosidase |
| Leber's hereditary optic neuropathy | NADH dehydrogenase |
| Leiomyosarcoma | |
| Lennox-Gastaut syndrome | |
| Lesch-Nyhan syndrome | Hypoxanthine phosphoribosyltransferase 1 |
| Leukaemia | |
| Li-Fraumeni syndrome | TP53 |
| Lipoma | |
| Liposarcoma | |
| Liver cancer | |
| Long-chain 3-OH acyl-CoA dehydrogenase deficiency | Long-chain-3-hydroxyacyl-CoA dehydrogenase |
| Lower respiratory infections | |
| Lysosomal acid lipase deficiency | Lysosomal acid lipase |
| Macular degeneration | |
| Major depressive disorder | |
| Malignant fibrous histiocytoma | |
| Mantle cell lymphoma | |
| Maple syrup urine disease | 3-methyl-2-oxobutanoate dehydrogenase |
| Marfan syndrome | FBN1 |
| Maroteaux-Lamy syndrome (MPS VI) | N-acetylgalactosamine 4-sulfatase |
| Mastocytosis | |
| McArdle disease | Muscle glycogen phosphorylase |
| MECP2-related disorders | |
| MECP2-related Severe Neonatal Encephalopathy | |
| Medium-chain acyl-CoA dehydrogenase deficiency | Acyl-CoA dehydrogenase |
| Melanoma | Anti-CTLA4 mAb |
| Metachromatic leukodystrophy | Arylsulfatase A |
| Metastatic colorectal cancer, NSCLC, others | Anti-VEGF mAb |
| Methylmalonyl-CoA mutase deficiency | Methylmalonyl-CoA mutase |
| Migraine | |
| Mitochondrial oxidative phosphorylation disorders | |
| Morquio syndrome, type A (MPS IVA) | Galactose 6-sulfate sulfatase |
| Morquio syndrome, type B (MPS IVB) | Beta-galactosidase |
| Mouth and oropharynx cancers | |
| Multiple carboxylase deficiency | Biotin-methylcrotonoyl-CoA-carboxylase ligase |
| Multiple myeloma | |
| Multiple sclerosis | Anti-VLA-4 mAb |
| Multiple sclerosis | Interferon beta |
| Multiple system atrophy | |
| Myasthenia gravis | |
| Myelofibrosis | |
| Narcolepsy | |
| Neonatal bronchopulmonary dysplasia | |
| Neonatal infections | |
| Nephritis and nephrosis | |
| Neurofibromatosis, type 1 | NF-1 |
| Neuronal ceroid lipofuscinoses-related diseases | |
| Neutropenia | G-CSF |
| Niemann Pick disease, type A/B | SMPD1 |
| Niemann Pick disease, type C | NPC1 |
| Niemann-Pick disease Type C1 | |
| Nocturia | |
| Non-alcoholic fatty liver disease | |
| Non-Hodgkin lymphoma | Anti-CD20 mAb |
| Non-small cell lung cancer | |
| Notch-3 related cerebral autosomal dominant | |
| arteriopathy with subcortical infarcts and | |
| leukoencephalopathy (CADASIL) | |
| Obesity | |
| Ophthalmoparesis | |
| Opioid induced constipation | |
| Ornithine transcarbamylase deficiency | Ornithine transcarbamylase |
| Osteoarthritis | |
| Osteopetrosis | |
| Osteoporosis | Anti-RANKL mAb |
| Ovarian cancer | |
| Paget disease of bone | Sequestosome 1 |
| Pain | |
| Pancreatic carcinoma | |
| Panic disorder | |
| Parkinson disease | |
| Paroxysmal nocturnal hemoglobinuria | Anti-complement factor C5 Mab |
| Pediculosis capitis (head lice) | |
| Pelizaeus-Merzbacher disease | |
| Pemphigus vulgaris | |
| Peptic ulcer disease | |
| Peripheral neuropathy | |
| Peyronie's disease | |
| Phenylketonuria | Phenylalanine hydroxylase |
| Pneumococcal infection prophylaxis | |
| POLG-related sensory ataxic neuropathy | |
| Polycystic kidney disease | |
| Polycystic ovary syndrome | |
| Polycythaemia vera | |
| Polymerase G-related disorders | |
| Polymorphous light eruption | |
| Pompe disease | Alpha glucosidase |
| Porphyria cutanea tarda | Uroporphyrinogen decarboxylase |
| Post herpetic neuralgia | |
| Post-organ transplant | |
| Pouchitis | |
| PPM-X Syndrome | |
| Prader-Willi syndrome | |
| Preeclampsia | |
| Premature ejaculation | |
| Prematurity and low birth weight | |
| Primary ciliary dyskinesia | |
| Primary glomerular diseases | |
| Primary humoral immune deficiencies (e.g., CVID) | Immunoglobulin |
| Proctitis | |
| Progressive multifocal leukoencephalopathy | |
| Progressive supranuclear palsy | |
| Propionic acidemia | Propionyl-CoA carboxylase |
| Prostate cancer | |
| Psoriasis | Anti-IL-12 & IL-23 mAb |
| Psoriatic arthritis | TNF-alpha inhibitors |
| PTT-1 | |
| Pulmonary arterial hypertension | |
| Pulmonary arterial hypertension | |
| Raynaud's phenomenon | |
| Refractive errors | |
| Renal cell carcinoma | |
| Restless leg syndrome | |
| Retinitis pigmentosa | |
| Rheumatic heart disease | |
| Rheumatoid arthritis | Anti-interleukin-6 (IL-6) mAb |
| Rheumatoid arthritis | T-cell costimulation blocker |
| Rheumatoid arthritis | TNF-alpha inhibitor |
| Romano-Ward syndrome | |
| Rosacea | |
| Sanfilippo syndrome, type A (MPS IIIA) | Heparan N-sulfatase |
| Sanfilippo syndrome, type B (MPS IIIB) | N-acetyl-alpha-D-glucosaminidase |
| Santavuori-Haltia disease | |
| Schizophrenia | |
| Schnitzler syndrome | |
| Scleroderma | |
| SCN1A | |
| SCN1B-related seizure disorders | |
| Short-chain acyl-CoA dehydrogenase deficiency | Butyryl-CoA dehydrogenase |
| Sickle cell disease | Hemoglobin |
| SLC3A1-related disorders | |
| Small cell lung cancer | |
| SMN-1-related spinal muscular atrophy (SMA) | |
| Spinal muscular atrophy | Survival motor neuron protein |
| Squamous cell carcinoma of head and neck | |
| Stickler syndrome | |
| Stomach cancer | |
| Stroke prophylaxis | |
| Synovial sarcoma | |
| Systemic lupus erythematosus | Anti-BAFF |
| Systemic sclerosis | |
| Tetrahydrobiopterin-deficient hyperphenylalaninemia | Tetrahydrobiopterin |
| Thromboangiitis obliterans | |
| Thrombotic disorders | |
| Thyroid cancer | |
| TPP1 deficiencies | |
| Trachea, bronchus, lung cancers | |
| Tricuspid atresia | |
| TSC1 | |
| TSC2-related tuberous sclerosis | |
| Type 2 diabetes mellitus | Glucagon-like peptide 1 (GLP-1) agonist |
| Type 2 diabetes mellitus | Insulin |
| Tyrosinemia, type I | Fumarylacetoacetase |
| Ulcerative colitis | |
| Uterine fibroids | |
| Varicose veins | |
| Venous thromboembolism | |
| Very long-chain acyl-CoA dehydrogenase deficiency | Long-chain-acyl-CoA dehydrogenase |
| von Gierke's disease | Glucose-6-phosphatase |
| Von Hippel-Lindau disease | pVHL |
| Wegener granulomatosis | |
| Wilson disease | Wilson disease protein |
| X-Linked adrenal hypoplasia | |
| X-linked adrenoleukodystrophy | |
| X-linked agammaglobulinemia | Bruton's tyrosine kinase |

In some embodiments, the present invention is used to prevent, treat and/or cure a subject affected with a disease or disorder listed or associated with the proteins listed in Tables 1, 2, 3, or 4. In some embodiments, an mRNA encodes one or more of Cystic Fibrosis Transmembrane Conductance Regulator (CFTR), argininosuccinate synthetase (ASS1), Factor IX, survival motor neuron 1 (SMN1), or phenylalanine hydroxylase (PAH). In some embodiments, the present invention is used to prevent, treat and/or cure a subject affected with any one of cystic fibrosis, citrullinemia, hemophilia B, spinal muscular atrophy and phenylketonuria.

### EXAMPLES

While certain compounds, compositions and methods of the present invention have been described with specificity in accordance with certain embodiments, the following examples serve only to illustrate the compounds of the invention and are not intended to limit the same.

### ABBREVIATIONS

DCM Dichloromethane
DMAP 4-(Dimethylamino)pyridine
DMF N,N-Dimethylformamide
EDCl N-(3-Dimethylaminopropyl)-N′-ethylcarbodiimide
NHS N-Hydroxysuccinimide
RT Room temperature
TBS tert-Butyldimethylsilyl
THF Tetrahydrofuran

### Example 1. General Synthesis of Compounds of Formula A

Compounds described herein can be prepared according to the exemplary syntheses described herein, including as shown in Scheme 1 of Compound 6.

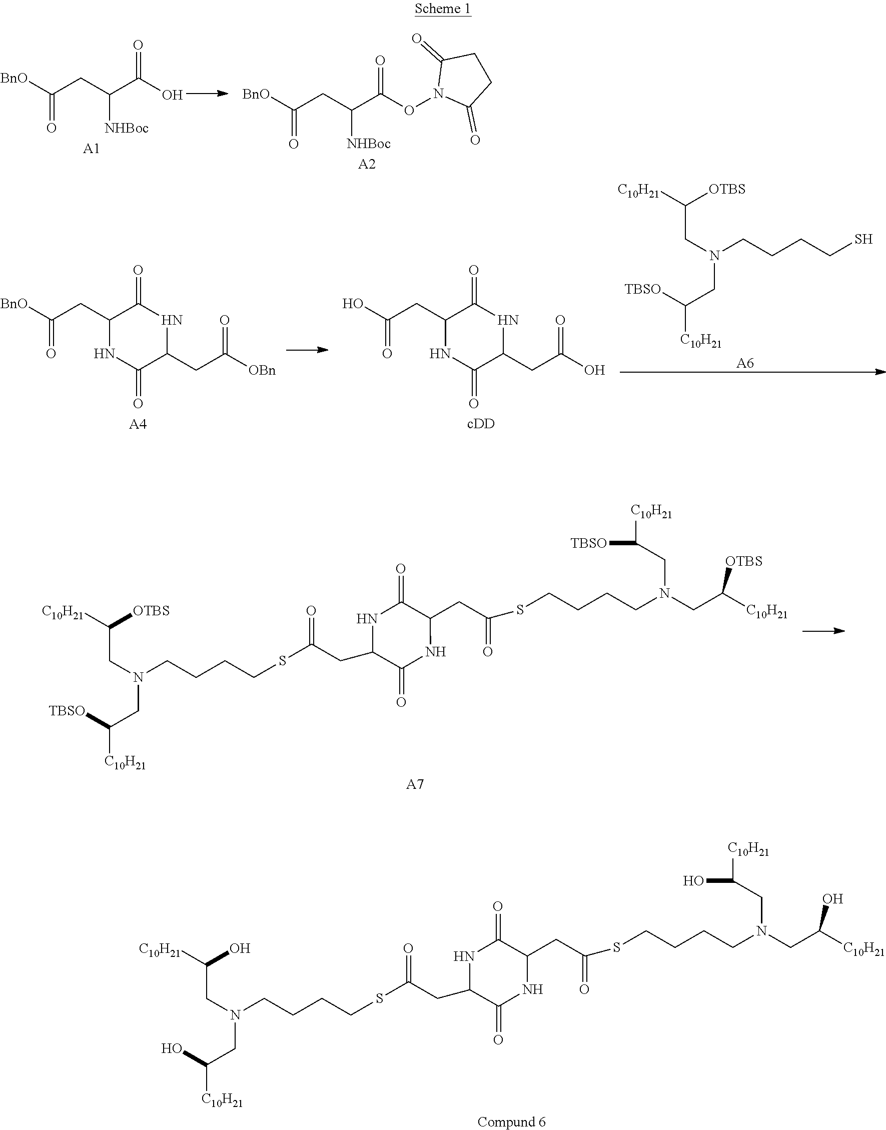

### Example 2. Exemplary Synthesis of cDD Thioester Lipid Compounds

cDD thioester lipids have been prepared, including Compounds 1, 3, 5, 6, 8, 9, 11, 12, 14, 15, 20, and 21.

### Compound 1

The exemplary synthesis of Scheme C can be used to prepare thioesters such as Compound 1.

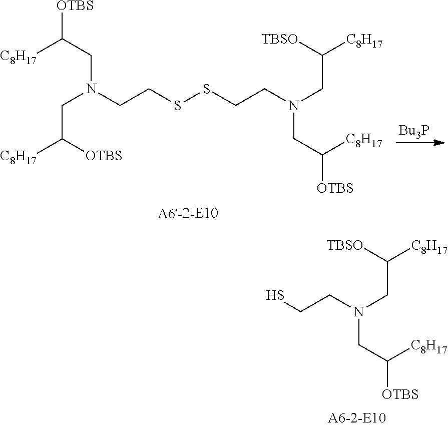

A precursor dimeric thiol (A6′-2-E10; 200 mg) was treated with reducing agent PBu₃ (2 hours at room temperature) to yield the monomeric thiol A6-2-E10, which was used in the next step without purification.

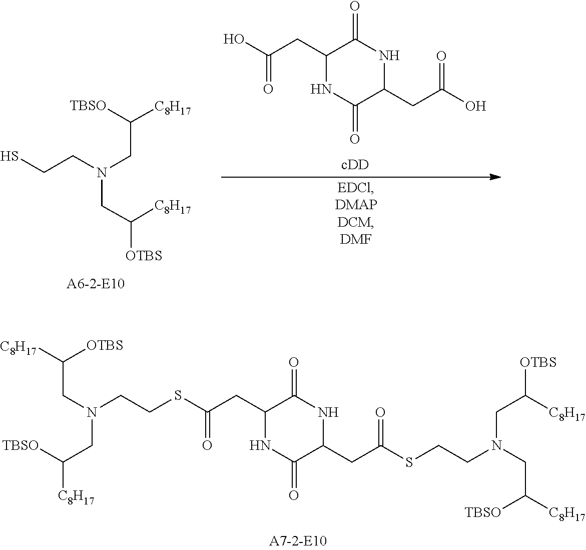

Crude thiol A6-2-E10 was then treated with cDD (37 mg) using EDCl/DMAP in DCM/DMF to provide the protected lipid A7-2-E10. Deprotection of lipid A7-2-E10 using HF in pyridine can afford Compound 1 (20 mg; 9% yield).

### Example 3. Exemplary Deprotection for Synthesis of cDD Ester Lipid Compound 39

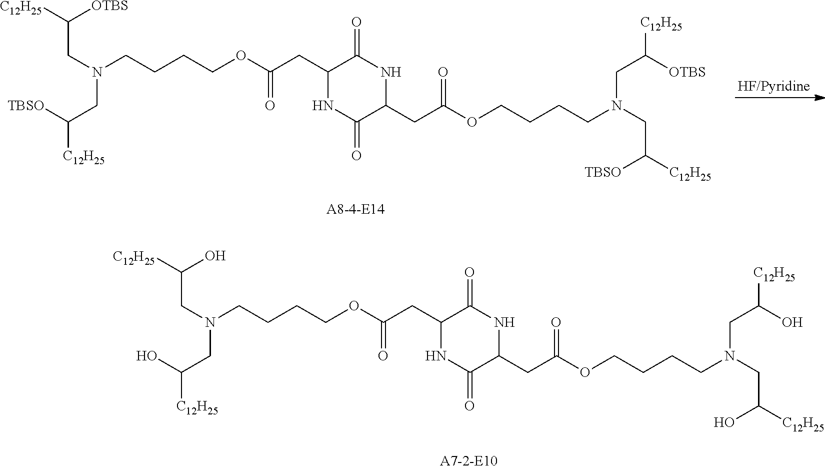

An exemplary deprotection of a protected cDD ester cationic lipid A8-4-E14 was accomplished using HF in pyridine (RT; 1 day) to afford the desired cDD ester cationic lipid Compound 39 (100 mg; 68% yield).

### Example 4. Exemplary Synthesis of cDD Ester Lipids

In addition to Compound 39, cDD ester lipid Compound 33 was prepared.

The exemplary synthesis of Scheme C was used to prepare Compound 33.

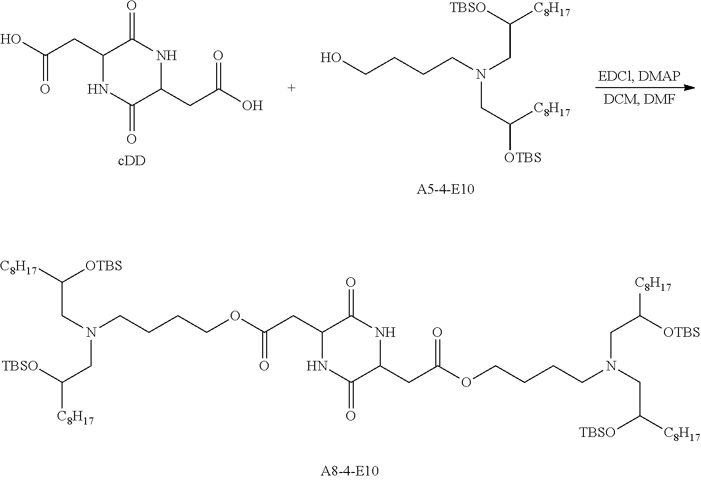

Diacid cDD (35 mg) was combined with protected alcohol A5-4-E10 using EDCl/DMAP in DCM/DMF (RT; 1 day) to yield the protected lipid A8-4-E10 (185 mg; 84% yield).

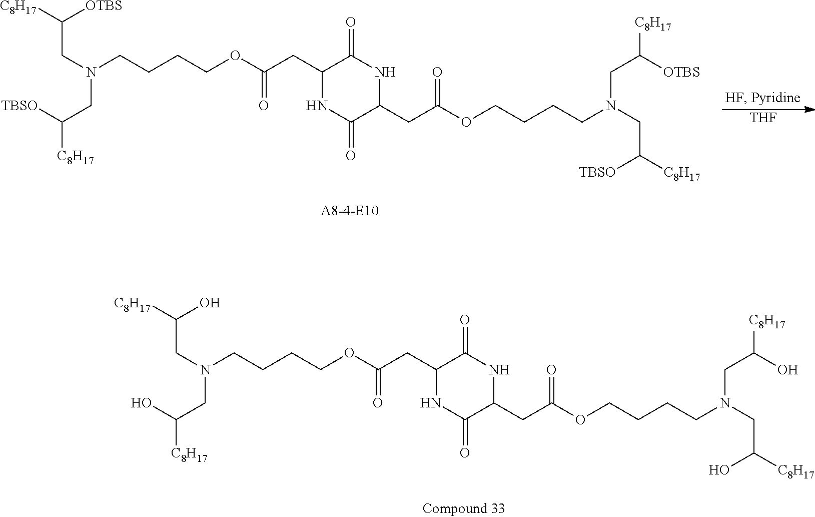

Protected lipid A8-4-E10 (185 mg) was treated with HF in pyridine/THF (RT; 1 day) to afford the desired cDD ester lipid Compound 33 (120 mg; 94%).

### Example 5. Exemplary Synthesis of cEE Thioester Lipids

cEE thioester lipids also have been prepared, including Compounds 63, 66, 69, 72, and 75.

Compound 72

The exemplary synthesis of Scheme B was used to prepare thioesters such as Compound 72.

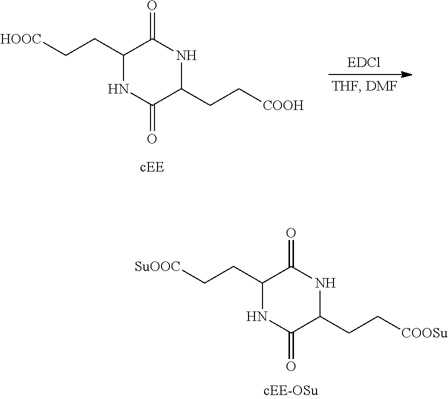

Diacid cEE was treated with NHS and EDCl in THF/DMF (RT; 1 day) to provide produce cEE-OSu in 85% yield.

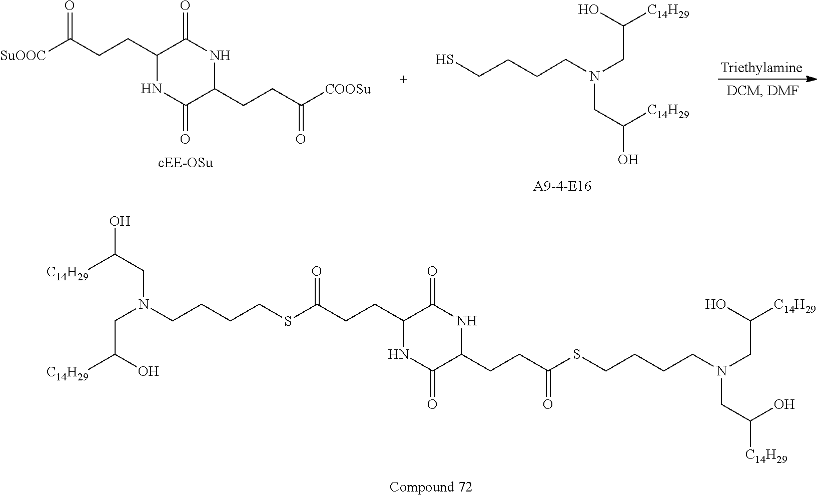

Activated intermediate cEE-OSu (500 mg) was treated with 1.3 g thiol A9-4-E16 (trimethylamine in DCM/DMF; 0° C. to RT overnight) to afford the desired cEE lipid Compound 72 (84 mg).

### Compound 75

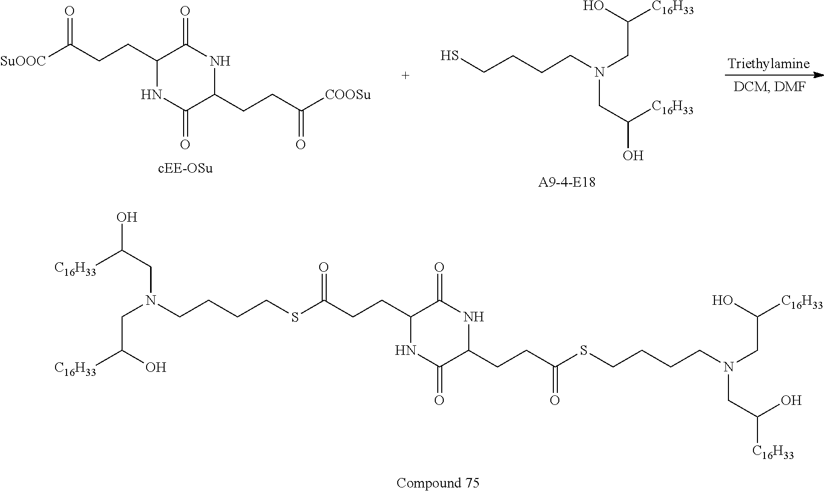

The procedure used to prepare Compound 72 was adapted to prepare cEE lipid Compound 75 (70 mg) by using thiol A9-4-E16.

This procedure also can be used to prepare other thioester lipids as shown in Table Q.

TABLE Q
Exemplary Lipids

| Target Product | Thiol | Conditions | Scale |
|---|---|---|---|
| Compound 74 | 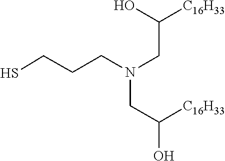 | DCM, DMF 0° C. to RT, overnight | cEE-OSu: 600 mg Thiol: 1.7 g |
| | A9-3-E18 | | |

### Example 6. Exemplary Synthesis of Homoserine (cHse) Lipids

Homoserine (cHse) lipids also have been prepared, including Compounds 121-129, 131-132, 134-135, and 140.

### Compound 122

The exemplary synthesis of Scheme D was used to prepare cHse lipids such as Compound 122.

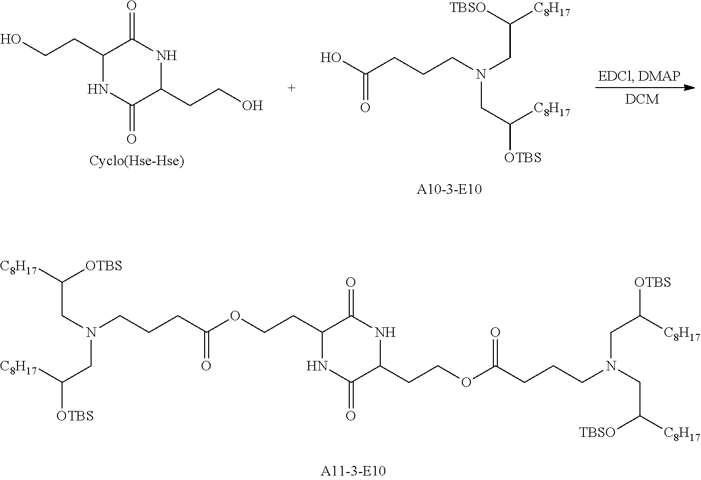

100 mg of dialcohol starting material cyclo(Hse-Hse) is treated with protected carboxylic acid A10-3-E10 (EDCl/DMAP in DCM; RT; overnight) to afford the protected cHse lipid A11-3-E10 (631 mg; 73% yield).

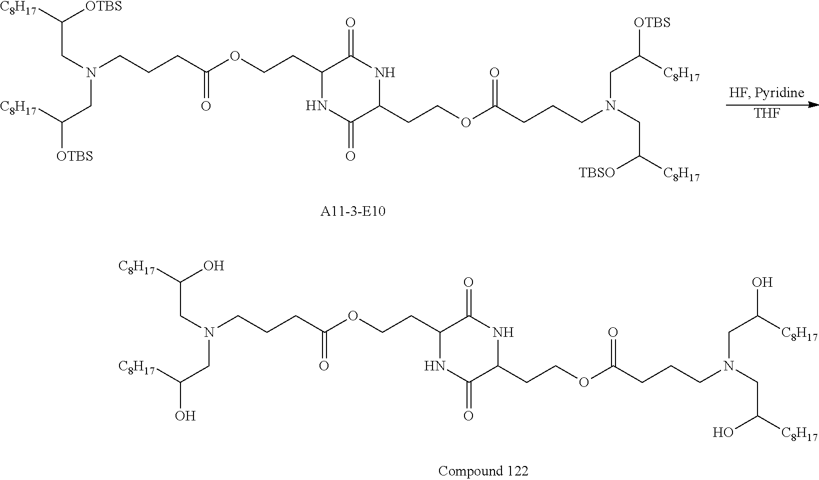

Intermediate A11-3-E10 (621 mg) was treated with HF in pyridine (RT; overnight) to provide the desired Compound 122 (326 mg; 77% yield).

### Compound 125

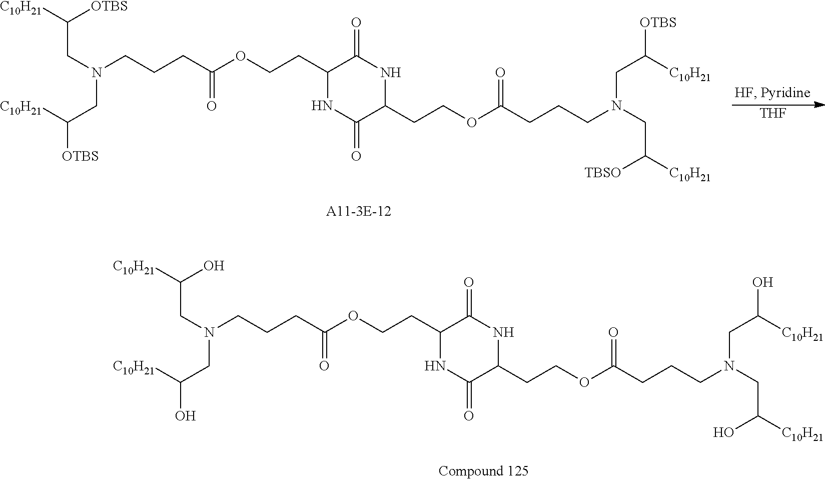

Deprotection of protected lipid A11-3-E12 (1.40 g) using HF/pyridine (RT; overnight) yielded Compound 125 (353 mg; 36% yield).

### Compound 135

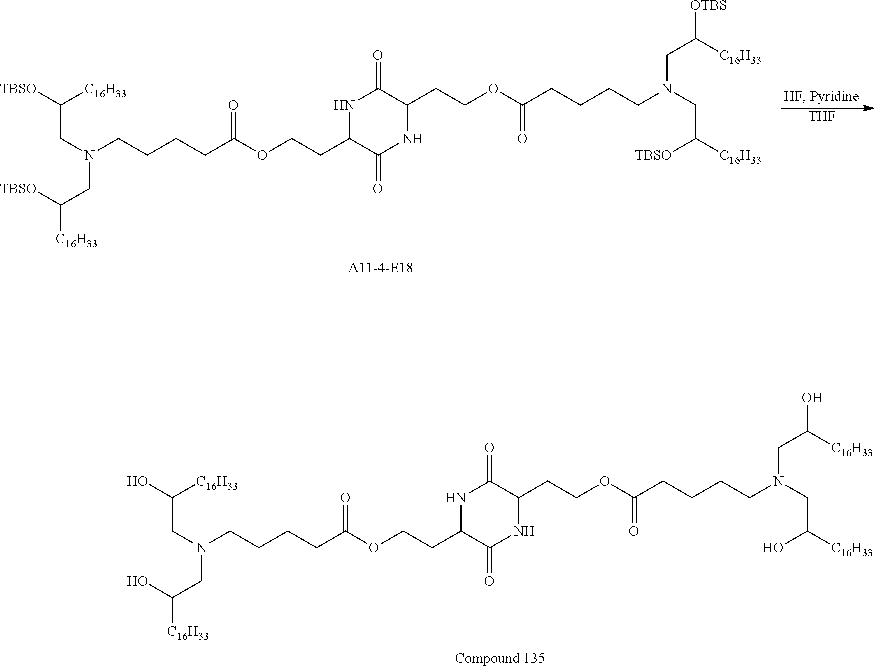

Deprotection of protected lipid A11-4-E18 (106 mg) using HF/pyridine (RT; overnight) yielded Compound 135 (106 mg; 41% yield).

### Example 7. Exemplary Synthesis of Serine (cSS) Lipids

### Synthesis of cSS-E-2-E12 [214]

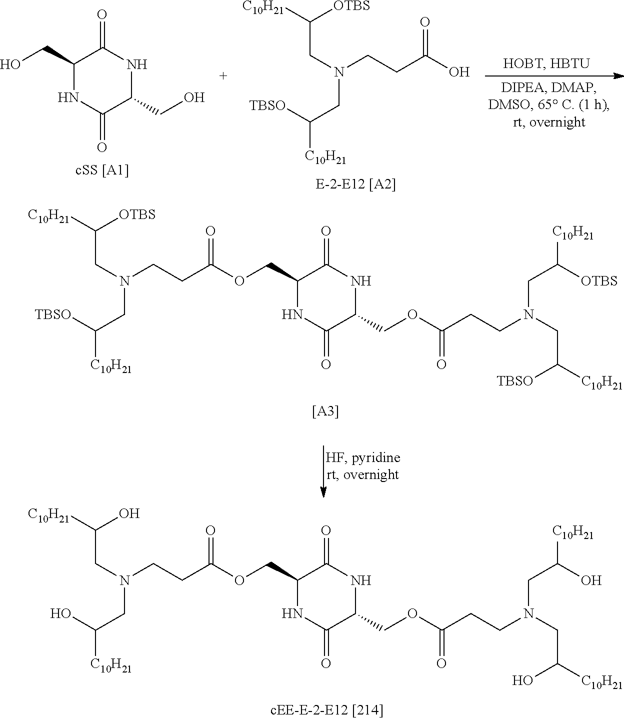

cSS [A1] (0.1 g, 0.57 mmol) and E-2-E12 [A2] (0.98 g, 1.44 mmol) in DMSO (10 mL) were added HOBt (0.23 g, 1.72 mmol), HBTU (0.65 g, 1.72 mmol), and DMAP (0.02 g, 0.172 mmol) followed by slow addition of DIPEA (1.0 mL, 5.75 mmol). The reaction was heated at 65° C. for 1 hour and continued stirring overnight at room temperature. Reaction mixture was then diluted with ethyl acetate (100 mL) and washed with brine solution (3×50 mL). After drying over anhydrous Na₂SO₄, the organic layer was evaporated under reduced pressure, and the residue was purified by silica gel chromatography (eluent: 0.2-0.5% MeOH in DCM) to obtain the compound [A3] as a light-yellow oil (0.60 g, 69%). Isolation of compound [A3] was confirmed based on MS analysis.

Compound A3 (0.2 g, 0.132 mmol) was then dissolved in to 4 mL dry THF in a 20 mL plastic scintillation vial, equipped with a Teflon stir-bar. The solution was then cooled to 0° C. using an ice bath. HF/pyridine (70 w/w %, 0.55 ml) was added dropwise into the reaction mixture and continued at room temperature overnight. The reaction mixture was then cooled to 5° C. and quenched with saturated sodium bicarbonate solution until the pH reached ^{˜}8-9. The mixtures were transferred to a separatory funnel and extracted with ethyl acetate (3×15 mL). The organic layers were combined, washed with brine solution (1×10 mL), dried with sodium sulfate, filtered, and concentrated on a to yield an off-yellow oil. This crude oil was subjected to Combi-flash purification using a 12 gram, 50 μm sized silica gel column chromatography (eluent: 2.0-5.0% MeOH in DCM). Purified product cSS-E-2-E12 [214] was obtained as colorless oil (80 mg, 57%).

ESI-MS analysis: Calculated C₆₀H₁₁₇N₄O₁₀, [M+H]=1053.88, Observed=1053.80

### Synthesis of cSS-E-2-E14 [217]

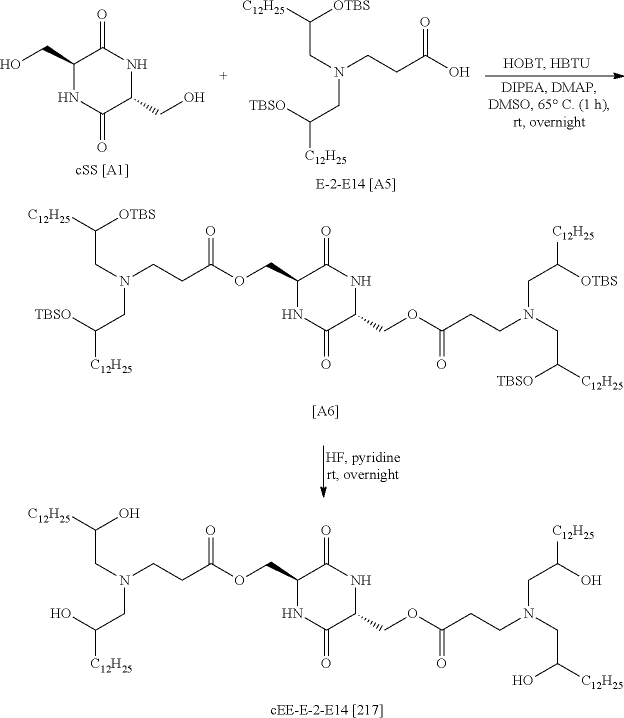

To a solution of cSS [A1] (0.1 g, 0.57 mmol) and E-2-E14 [A5] (0.94 g, 1.26 mmol) in DMSO (10 mL) were added HOBt (0.23 g, 1.72 mmol), HBTU (0.65 g, 1.72 mmol), and DMAP (0.02 g, 0.172 mmol) followed by slow addition of DIPEA (1.0 mL, 5.75 mmol). The reaction was heated at 65° C. for 1 hour and continued stirring overnight at room temperature. Reaction mixture was then diluted with ethyl acetate (100 mL) and washed with brine solution (3×50 mL). After drying over anhydrous Na₂SO₄, the organic layer was evaporated under reduced pressure, and the residue was purified by silica gel chromatography (eluent: 0.2-0.5% MeOH in DCM) to obtain the compound [A6] as a pale-yellow oil (0.56 g, 60%). Isolation of compound [A6] was confirmed based on MS analysis.

Compound A6 (0.175 g, 0.108 mmol) was then dissolved in to 4 mL dry THF in a 20 mL plastic scintillation vial, equipped with a Teflon stir-bar. The solution was then cooled to 0° C. using an ice bath. HF/pyridine (70 w/w %, 0.55 ml) was added dropwise into the reaction mixture and continued at room temperature overnight. The reaction mixture was then cooled to 5° C. and quenched with saturated sodium bicarbonate solution until the pH reached ^{˜}8-9. The mixtures were transferred to a separatory funnel and extracted with ethyl acetate (3×15 mL). The organic layers were combined, washed with brine solution (1×10 mL), dried with sodium sulfate, filtered, and concentrated on a to yield an off-yellow oil. This crude oil was subjected to Combi-flash purification using a 12 gram, 50 μm sized silica gel column chromatography (eluent: 2.0-5.0% MeOH in DCM). Purified product cSS-E-2-E14 [217] was obtained as colorless oil (50 mg, 40%).

ESI-MS analysis: Calculated C₆₈H₁₃₃N₄O₁₀, [M+H]=1166.0, Observed=1166.0

### Synthesis of cSS-E-2-Oi10 [550]

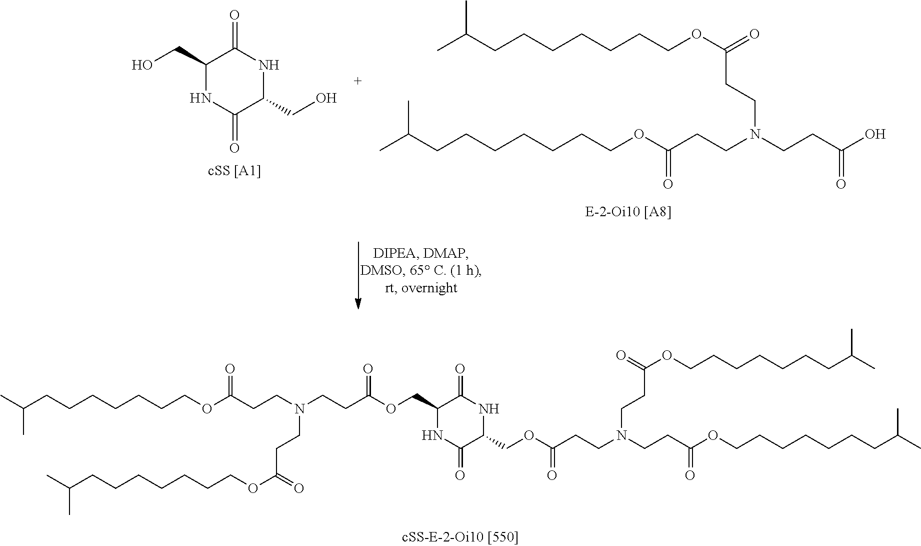 [SMILES: O=C1N[C@H](CO)C(=O)N[C@H]1CO ; CC(C)CCCCCCCOC(=O)CCN(CCC(=O)O)CCC(=O)OCCCCCCCC(C)C ; CC(C)CCCCCCCOC(=O)CCN(CCC(=O)OCCCCCCCC(C)C)CCC(=O)OC[C@@H]1NC(=O)[C@@H](COC(=O)CCN(CCC(=O)OCCCCCCCC(C)C)CCC(=O)OCCCCCCCC(C)C)NC1=O]

To a solution of cSS [A1] (0.1 g, 0.575 mmol) and E-2-Oi10 [A8] (0.65 g, 1.26 mmol) in DMSO (8 mL) were added HOBt (0.23 g, 1.72 mmol), HBTU (0.65 g, 1.72 mmol), and DMAP (0.02 g, 0.172 mmol) followed by slow addition of DIPEA (1.0 mL, 5.75 mmol). The reaction was heated at 65° C. for 1 hour and continued stirring overnight at room temperature. Reaction mixture was then diluted with ethyl acetate (100 mL) and washed with brine solution (3×50 mL). After drying over anhydrous Na₂SO₄, the organic layer was evaporated under reduced pressure, and the residue was purified by silica gel chromatography (eluent: 1.0-2.0% MeOH in DCM) to obtain the compound cSS-E-2-Oi10 [9] as a pale-yellow oil (0.25 g, 37%). Isolation of compound 550 was confirmed based on MS analysis.

ESI-MS analysis: Calculated C₆₄H₁₁₇N₄O₁₄, [M+H]=1165.8, Observed=1167.9

### Synthesis of cCC-SS-2-E10 [154]

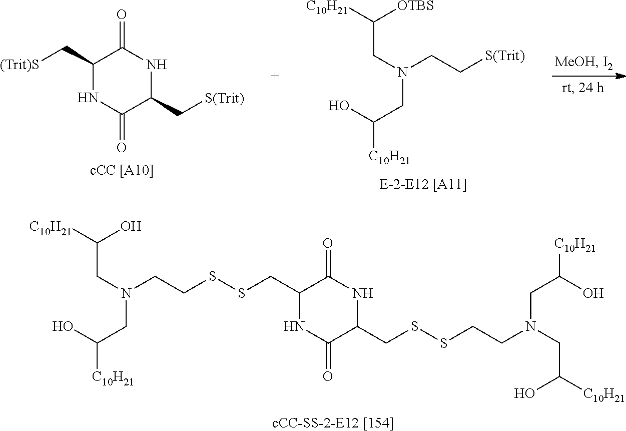

Trityl protected-cyclic Cystine, cCC [A10] (60 mg, 0.087 mmol) and S-2-E12 [A11] (180 mg, 0.261 mmol) in dry methanol (10 mL) was added dropwise to a rapidly stirred solution of Iodine (221 mg, 0.87 mmol) in dry methanol. At 0° C. for 1 h and then continued stirring at room temperature for 24 h. The reaction was quenched with 1N Na₂S₂O₃ solution (5 mL) unless a nearly colorless solution was obtained. The reaction mixture was evaporated off to get rid of methanol and then extracted with ethyl acetate (2×25 mL). The combined EtOAc layer was further washed with 0.1N Na₂S₂O₃ (10 mL). After drying over anhydrous Na₂SO₄, the organic layer was evaporated under reduced pressure, and the residue was purified by silica gel chromatography (eluent: 1.0-3.0% MeOH in DCM) to obtain the compound 154 as a light brown oil (69.7 mg, 72%). Isolation of compound cCC-SS-2-E12 [154] was confirmed based on MS analysis.

ESI-MS analysis: Calculated C₅₈H₁₁₆N₄O₆S₄. Na⁺, [M+Na⁺]=1115.77, Observed=1115.50

### Example 8. Exemplary Methods for Preparation of Lipid Nanoparticles

Cationic lipids described herein can be used in the preparation of lipid nanoparticles according to methods known in the art. For example, suitable methods include methods described in International Publication No. WO 2018/089801, which is hereby incorporated by reference in its entirety.

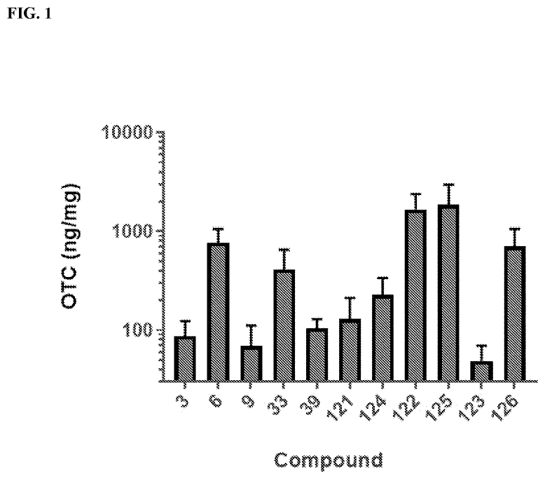

One exemplary process for lipid nanoparticle formulation is Process A of WO 2018/089801 (see, e.g., Example 1 and FIG. 1 of WO 2018/089801). Process A (“A”) relates to a conventional method of encapsulating mRNA by mixing mRNA with a mixture of lipids, without first pre-forming the lipids into lipid nanoparticles. In an exemplary process, an ethanol lipid solution and an aqueous buffered solution of mRNA were prepared separately. A solution of mixture of lipids (cationic lipid, helper lipids, zwitterionic lipids, PEG lipids etc.) was prepared by dissolving lipids in ethanol. The mRNA solution was prepared by dissolving the mRNA in citrate buffer. The mixtures were then both heated to 65° C. prior to mixing. Then, these two solutions were mixed using a pump system. In some instances, the two solutions were mixed using a gear pump system. In certain embodiments, the two solutions were mixing using a ‘T’ junction (or “Y” junction). The mixture was then purified by diafiltration with a TFF process. The resultant formulation concentrated and stored at 2-8° C. until further use.

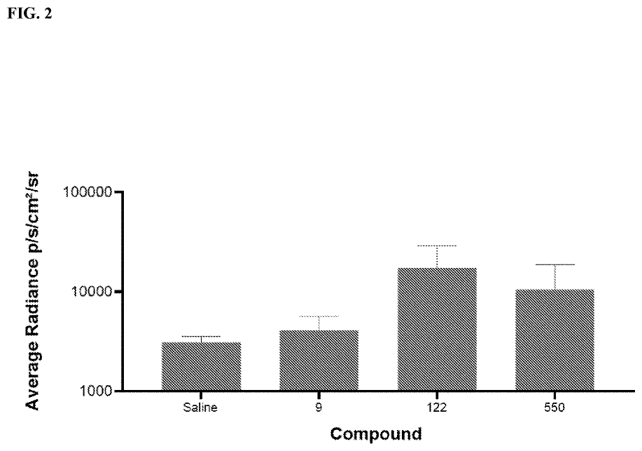

A second exemplary process for lipid nanoparticle formulation is Process B of WO 2018/089801 (see, e.g., Example 2 and FIG. 2 of WO 2018/089801). Process B (“B”) refers to a process of encapsulating messenger RNA (mRNA) by mixing pre-formed lipid nanoparticles with mRNA. A range of different conditions, such as varying temperatures (i.e., heating or not heating the mixture), buffers, and concentrations, may be employed in Process B. In an exemplary process, lipids dissolved in ethanol and citrate buffer were mixed using a pump system. The instantaneous mixing of the two streams resulted in the formation of empty lipid nanoparticles, which was a self-assembly process. The resultant formulation mixture was empty lipid nanoparticles in citrate buffer containing alcohol. The formulation was then subjected to a TFF purification process wherein buffer exchange occurred. The resulting suspension of pre-formed empty lipid nanoparticles was then mixed with mRNA using a pump system. For certain cationic lipids, heating the solution post-mixing resulted in a higher percentage of lipid nanoparticles containing mRNA and a higher total yield of mRNA.

Lipid nanoparticle formulations of Table R were prepared by Process B. All of the lipid nanoparticle formulations for comprised mRNA encoding ornithine transcarbamylase protein (hOTC mRNA) and lipids (Cationic Lipid: DMG-PEG2000; Cholesterol: DOPE or DEPE) in the mol % ratios set forth in Table R.

TABLE R
Exemplary lipid nanoparticle formulations for intravenous administration

| | Composition (mol %) | | | | | |
|---|---|---|---|---|---|---|
| | (Cationic Lipid: DMG- | | | | | |
| Cationic Lipid | PEG2000; Cholesterol: | Helper | | Size | | Encapsulation |
| Compound | Helper Lipid) | Lipid | N/P | (nm) | PDI | % |
| 3 | 40:3:25:32 | DOPE | 4 | 114 | 0.15 | 87 |
| 6 | 40:3:25:32 | DEPE | 4 | 120 | 0.17 | 92 |
| 9 | 40:3:27:30 | DEPE | 4 | 166 | 0.21 | 71 |
| 33 | 40:3:25:32 | DOPE | 4 | 103 | 0.16 | 93 |
| 39 | 40:3:25:32 | DEPE | 4 | 155 | 0.17 | 93 |
| 121 | 40:3:25:32 | DOPE | 4 | 103 | 0.19 | 82 |
| 124 | 40:3:25:32 | DOPE | 4 | 112 | 0.21 | 81 |
| 122 | 40:3:27:30 | DOPE | 4 | 119 | 0.16 | 83 |
| 125 | 40:3:25:32 | DEPE | 4 | 116 | 0.11 | 98 |
| 123 | 40:3:27:30 | DOPE | 4 | 109 | 0.19 | 67 |
| 126 | 40:3:27:30 | DOPE | 4 | 122 | 0.17 | 81 |

The lipid nanoparticle formulations of Table S were prepared by either Process A or B. Each formulation comprised mRNA encoding firefly luciferase protein (FFL mRNA) and lipids (Cationic Lipid: DMG-PEG2000; Cholesterol: DOPE) in the mol % ratios set forth in Table S.

TABLE S
Exemplary lipid nanoparticle formulations for intratracheal administration

| | | Composition (mol %) | | | | |
|---|---|---|---|---|---|---|
| | | (Cationic Lipid: DMG- | | | | |
| Cationic Lipid | | PEG2000; Cholesterol: | | Size | | Encapsulation |
| Compound | Process | DOPE) | N/P | (nm) | PDI | % |
| 9 | B | 40:5:25:30 | 4 | 116 | 0.16 | 80 |
| 122 | B | 40:5:25:30 | 4 | 131 | 0.20 | 65 |
| 550 | A | 40:5:25:30 | 4 | 118 | 0.10 | 81 |

### Example 9. In Vivo Expression of hOTC in CD1 Mice

Intravenous (IV) administration of lipid nanoparticle formulations comprising a cationic lipid and hOTC mRNA (Table R) was undertaken in order to study mRNA delivery and resultant hOTC protein expression. Male CD1 mice at 6-8 weeks old were given a single bolus tail-vein injection of the LNP formulations at a dose of 1 mg/kg. The mice were sacrificed and perfused with saline 24 hours post-administration. Liver tissue was collected, and hOTC protein expression levels were measured in liver homogenate by ELISA. As shown in FIG. 1, the cationic lipids described herein were effective in delivering mRNA in vivo and resulted in expression of protein encoded by the delivered mRNA.

### Example 10. Delivery of FFL mRNA by Intratracheal Administration

Lipid nanoparticle formulations comprising FFL mRNA in Table S were administered to male CD1 mice (6-8 weeks old) by a single intratracheal aerosol administration via a Microsprayer® (50 ul/animal) while under anesthesia. Intratracheal aerosol administration via a Microsprayer® is a suitable model for pulmonary delivery via nebulization. At approximately 24 hours post-dose, the animals were dosed with luciferin at 150 mg/kg (60 mg/ml) by intraperitoneal injection at 2.5 ml/kg. After 5-15 minutes, all animals were imaged using an IVIS imaging system to measure luciferase production in the lung. FIG. 2 shows that lipid nanoparticles comprising the cationic lipids descried herein are also effective in delivering mRNA to the lung based on positive luciferase activity.

While certain compounds, compositions and methods of the present invention have been described with specificity in accordance with certain embodiments, the disclosed examples serve only to illustrate the compounds of the invention and are not intended to limit the same.

What is claimed is:

1. A cationic lipid having the following structure: 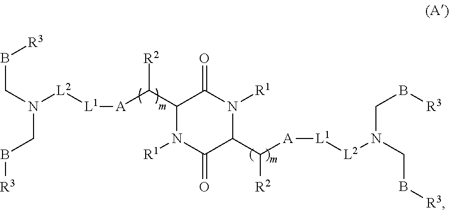
or a pharmaceutically acceptable salt thereof, wherein
each R¹ and R² is independently H or C₁-C₆ aliphatic;
each m is independently an integer having a value of 1 to 4;
each A is independently a covalent bond or arylene;
each L¹ is independently an ester, thioester, disulfide, or anhydride group;
each L² is independently C₂-C₁₀ aliphatic;
each B is independently —CHX¹— or —CH₂CO₂—;
each X¹ is independently H or OH; and
each R³ is independently C₆-C₃₀ aliphatic.

2. The cationic lipid of claim 1, having the following structure: 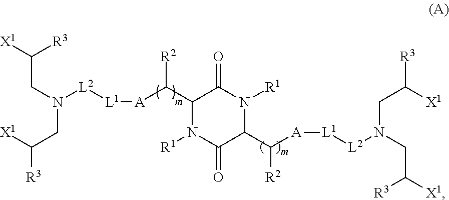
or a pharmaceutically acceptable salt thereof, wherein
each R¹ and R² is independently H or C₁-C₆ aliphatic;
each m is independently an integer having a value of 1 to 4;
each A is independently a covalent bond or arylene;
each L¹ is independently an ester, thioester, disulfide, or anhydride group;
each L² is independently C₂-C₁₀ aliphatic;
each X¹ is independently H or OH; and
each R³ is independently C₆-C₃₀ aliphatic.

3. The cationic lipid of claim 1 or 2, wherein each A is independently a covalent bond or phenylene.

4. The cationic lipid of any one of claims 1-3, having the following structure, 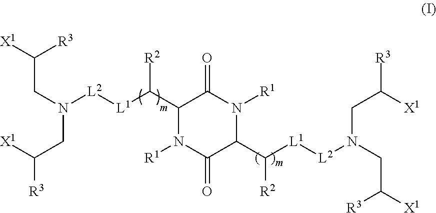
or a pharmaceutically acceptable salt thereof.

5. The cationic lipid of any one of claims 1-4, wherein each R¹ is H.

6. The cationic lipid of any one of claims 1-5, wherein each R² is independently H or C₁-C₆ alkyl.

7. The cationic lipid of any one of claims 1-6, wherein each L² is independently C₂-C₁₀ alkylene.

8. The cationic lipid of any one of claims 1-7, wherein each R³ is independently C₆-C₂₀ alkyl, C₆-C₂₀ alkenyl, or C₆-C₂₀ alkynyl.

9. The cationic lipid of claim 8, wherein said R³ comprises a substituent that is —O—C(O)R′ or —C(O)—OR′, wherein R′ is C₁-C₁₆ alkyl.

10. The cationic lipid of any one of claims 1-9, wherein each X¹ is OH.

11. The cationic lipid of any one of claims 1-10, wherein each m is 1.

12. The cationic lipid of any one of claims 1-10, wherein each m is 2.

13. The cationic lipid of any one of claims 1-10, wherein each m is 3.

14. The cationic lipid of any one of claims 1-10, wherein each m is 4.

15. The cationic lipid of claim 11, having the following structure: 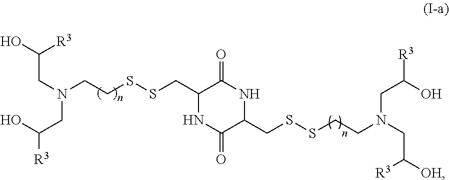
or a pharmaceutically acceptable salt thereof, wherein each n is independently an integer having a value from 1 to 9.

16. The cationic lipid of claim 15, having the following structure: 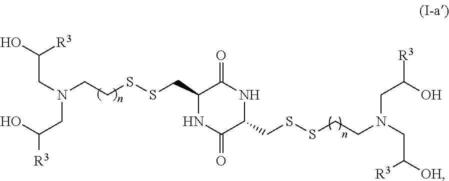
or a pharmaceutically acceptable salt thereof.

17. The cationic lipid of claim 15 or 16, wherein (i) each n is 1; (ii) each n is 2; or (iii) each n is 3.

18. The cationic lipid of claim 11, having the following structure: 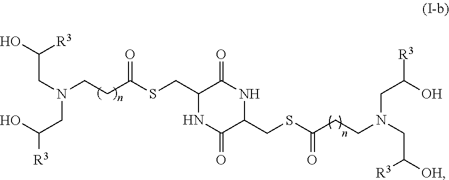
or a pharmaceutically acceptable salt thereof, wherein each n is an integer having a value of 1 to 9.

19. The cationic lipid of claim 18, having the following structure: 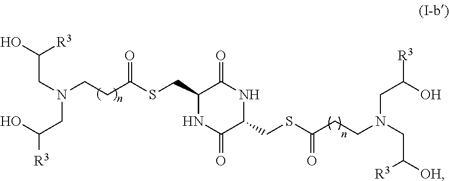
or a pharmaceutically acceptable salt thereof.

20. The cationic lipid of claim 18 or 19, wherein (i) each n is 1; (ii) each n is 2; or (iii) each n is 3.

21. The cationic lipid of claim 11, having the following structure: 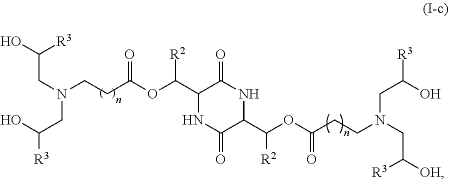
or a pharmaceutically acceptable salt thereof, wherein
each n is an integer having a value of 1 to 9; and
each R² is independently H or CH₃.

22. The cationic lipid of claim 21, having the following structure: 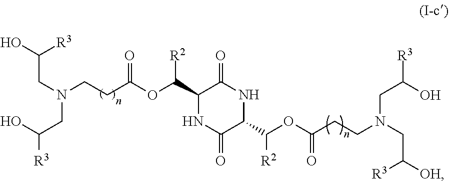
or a pharmaceutically acceptable salt thereof.

23. The cationic lipid of claim 21 or 22, wherein each R² is H.

24. The cationic lipid of claim 23, having the following structure: 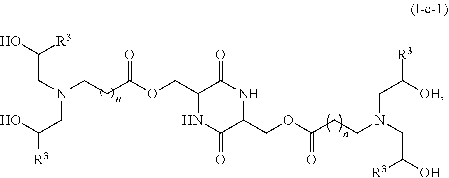
or a pharmaceutically acceptable salt thereof.

25. The cationic lipid of claim 23 or 24, having the following structure: 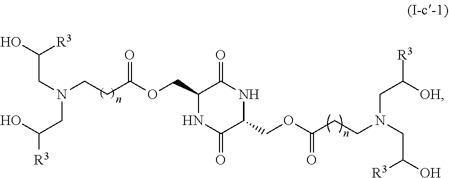
or a pharmaceutically acceptable salt thereof.

26. The cationic lipid of claim 21 or 22, wherein each R² is CH₃.

27. The cationic lipid of claim 26, having the following structure: 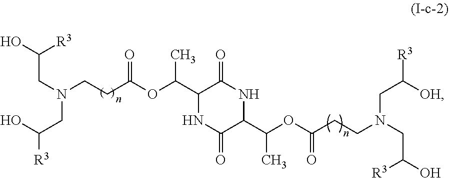
or a pharmaceutically acceptable salt thereof, optionally wherein the cationic lipid has the following structure: 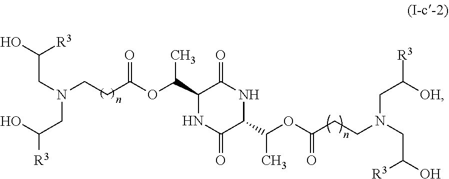
or a pharmaceutically acceptable salt thereof.

28. The cationic lipid of any one of claims 21-27, wherein (i) each n is 1; (ii) each n is 2; or (iii) each n is 3.

29. The cationic lipid of claim 11, having the following structure: 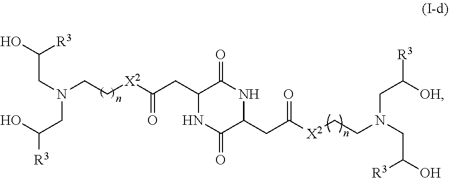
or a pharmaceutically acceptable salt thereof, wherein
each n is independently an integer having a value of 1 to 9; and
each X² is independently O or S.

30. The cationic lipid of claim 29, having the following structure: 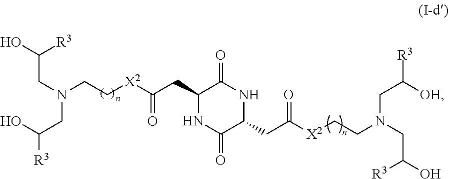
or a pharmaceutically acceptable salt thereof.

31. The cationic lipid of claim 29 or 30, wherein each n is 1.

32. The cationic lipid of claim 29 or 30, wherein each n is 2.

33. The cationic lipid of claim 29 or 30, wherein each n is 3.

34. The cationic lipid of any one of claims 29-33, wherein each X² is S.

35. The cationic lipid of claim 34, having the following structure, 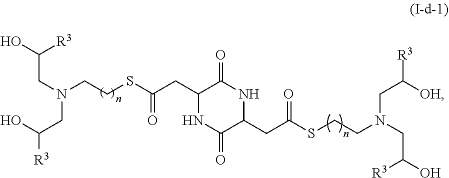
or a pharmaceutically acceptable salt thereof.

36. The cationic lipid of any one of claims 29-33, wherein each X² is O.

37. The cationic lipid of claim 36, having the following structure, 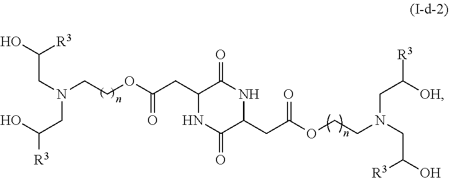
or a pharmaceutically acceptable salt thereof.

38. The cationic lipid of claim 12, having the following structure: 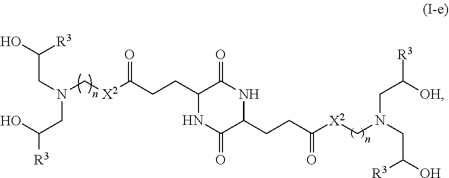
or a pharmaceutically acceptable salt thereof, wherein
each n is independently an integer of having a value of 2 to 10; and
each X² is independently 0 or S.

39. The cationic lipid of claim 38, having the following structure: 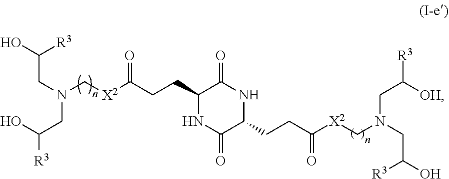
or a pharmaceutically acceptable salt thereof.

40. The cationic lipid of claim 38 or 39, wherein each n is 2.

41. The cationic lipid of claim 38 or 39, wherein each n is 3.

42. The cationic lipid of claim 38 or 39, wherein each n is 4.

43. The cationic lipid of any one of claims 38-42, wherein each X² is S.

44. The cationic lipid of claim 43, having the following structure, 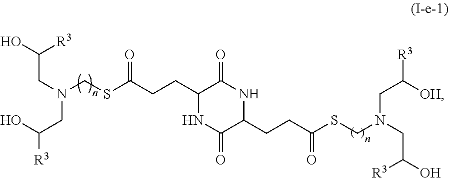
or a pharmaceutically acceptable salt thereof.

45. The cationic lipid of any one of claims 38-42, wherein each X² is O.

46. The cationic lipid of claim 45, having the following structure, 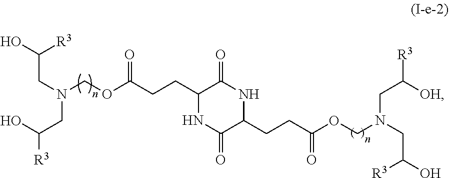
or a pharmaceutically acceptable salt thereof.

47. The cationic lipid of claim 12, having the following structure: 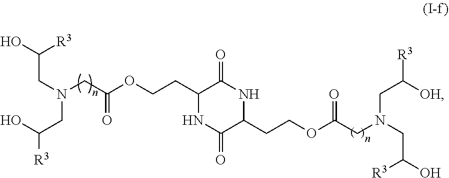
or a pharmaceutically acceptable salt thereof, wherein
each n is independently an integer of having a value of 2 to 10.

48. The cationic lipid of claim 47, having the following structure: 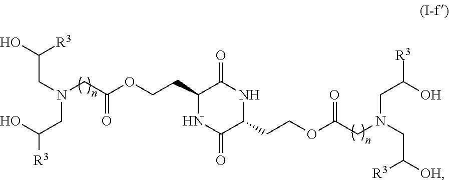
or a pharmaceutically acceptable salt thereof.

49. The cationic lipid of claim 47 or 48, wherein each n is 2.

50. The cationic lipid of claim 47 or 48, wherein each n is 3.

51. The cationic lipid of claim 47 or 48, wherein each n is 4.

52. The cationic lipid of any one of claims 1 to 51, wherein each R³ is independently C₆-C₂₀ aliphatic.

53. The cationic lipid of any one of claims 1-3, having the following structure: 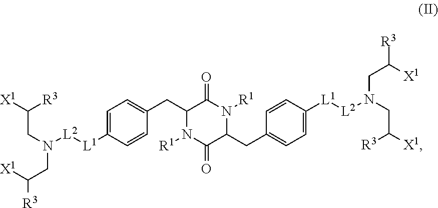
or a pharmaceutically acceptable salt thereof, wherein
each R¹ is independently H or C₁-C₆ aliphatic;
each L¹ is independently an ester, thioester, disulfide, or anhydride group;
each L² is independently C₂-C₁₀ aliphatic;
each X¹ is independently H or OH; and
each R³ is independently C₆-C₃₀ aliphatic.

54. The cationic lipid of claim 53, wherein each R¹ is independently H or C₁-C₆ alkyl.

55. The cationic lipid of claim 53 or 54, wherein each R¹ is H.

56. The cationic lipid of any one of claims 53-55, wherein each X¹ is OH.

57. The cationic lipid of any one of claims 53-56, having the following structure: 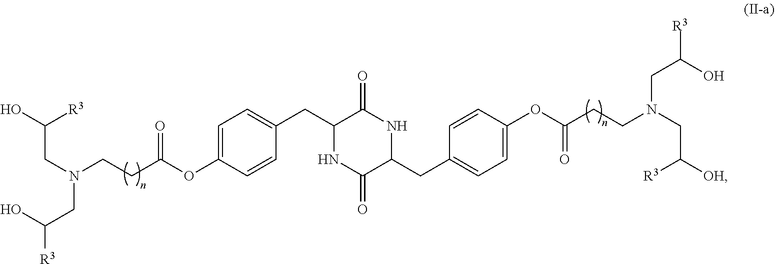
or a pharmaceutically acceptable salt thereof, wherein each n is an integer having a value of 1 to 9.

58. The cationic lipid of claim 57, having the following structure: 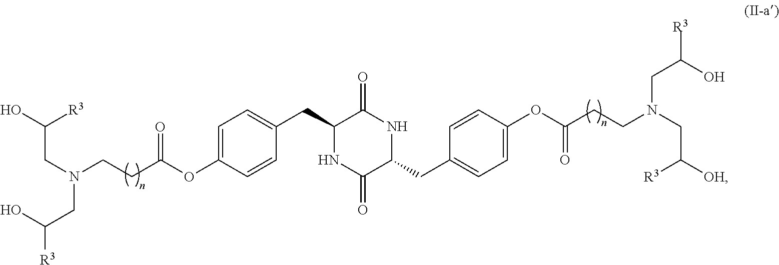
or a pharmaceutically acceptable salt thereof.

59. The cationic lipid of claim 57 or 58, wherein each n is 2.

60. The cationic lipid of any one of claims 53-59, wherein each R³ is independently C₈-C₂₀ aliphatic.

61. The cationic lipid of claim 1, having the following structure: 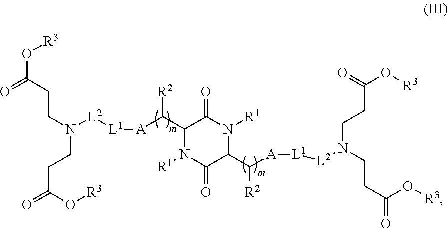
or a pharmaceutically acceptable salt thereof, wherein
each R¹ and R² is independently H or C₁-C₆ aliphatic;
each m is independently an integer having a value of 1 to 4;
each A is independently a covalent bond or arylene;
each L¹ is independently an ester, thioester, disulfide, or anhydride group;
each L² is independently C₂-C₁₀ aliphatic;
each R³ is independently C₆-C₃₀ aliphatic.

62. The cationic lipid of claim 60, wherein each A is independently a covalent bond or phenylene.

63. The cationic lipid of claim 61 or 62, having the following structure, 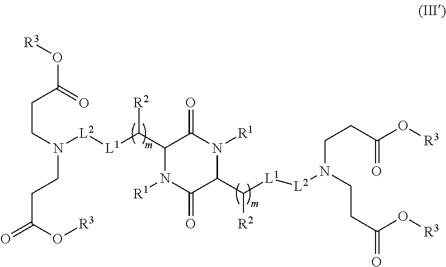
or a pharmaceutically acceptable salt thereof.

64. The cationic lipid of any one of claims 61-63, wherein each R¹ is H.

65. The cationic lipid of any one of claims 61-64, wherein each R² is independently H or C₁-C₆ alkyl.

66. The cationic lipid of any one of claims 61-65, wherein each L² is independently C₂-C₁₀ alkylene.

67. The cationic lipid of any one of claims 61-66, wherein each R³ is independently C₆-C₂₀ alkyl, C₆-C₂₀ alkenyl, or C₆-C₂₀ alkynyl.

68. The cationic lipid of claim 67, wherein said R³ comprises a substituent that is —O—C(O)R′ or —C(O)—OR′, wherein R′ is C₁-C₁₆ alkyl.

69. The cationic lipid of any one of claims 61-68, wherein each m is 1.

70. The cationic lipid of any one of claims 61-68, wherein each m is 2.

71. The cationic lipid of any one of claims 61-68, wherein each m is 3.

72. The cationic lipid of any one of claims 61-68, wherein each m is 4.

73. The cationic lipid of any one of claims 61-63, having the following structure: 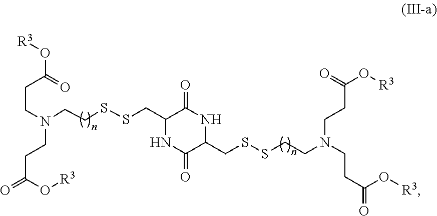
or a pharmaceutically acceptable salt thereof, wherein each n is independently an integer having a value from 1 to 9.

74. The cationic lipid of claim 73, having the following structure: 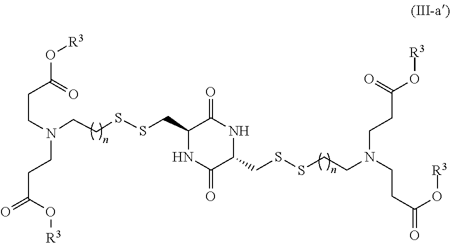
or a pharmaceutically acceptable salt thereof.

75. The cationic lipid of claim 73 or 74, wherein (i) each n is 1; (ii) each n is 2; or (iii) each n is 3.

76. The cationic lipid of any one of claims 61-63, having the following structure: 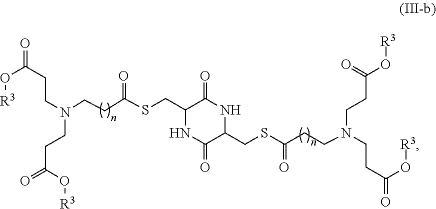
or a pharmaceutically acceptable salt thereof, wherein each n is an integer having a value of 1 to 9.

77. The cationic lipid of claim 76, having the following structure: 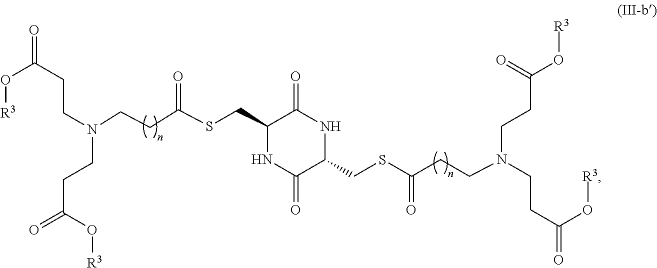
or a pharmaceutically acceptable salt thereof.

78. The cationic lipid of claim 76 or 77, wherein (i) each n is 1; (ii) each n is 2; or (iii) each n is 3.

79. The cationic lipid of any one of claims 61-63, having the following structure: 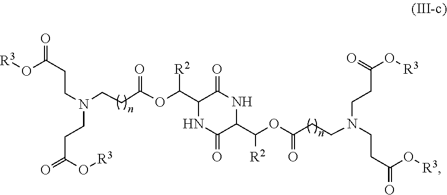
or a pharmaceutically acceptable salt thereof, wherein
each n is an integer having a value of 1 to 9; and
each R² is independently H or CH₃.

80. The cationic lipid of claim 79, having the following structure: 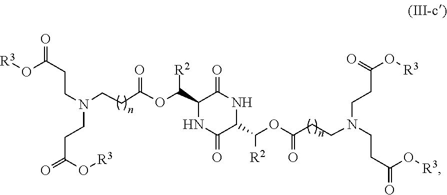
or a pharmaceutically acceptable salt thereof.

81. The cationic lipid of claim 79 or 80, wherein each R² is H.

82. The cationic lipid of claim 81, having the following structure: 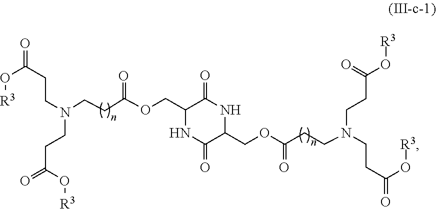
or a pharmaceutically acceptable salt thereof.

83. The cationic lipid of claim 82, having the following structure: 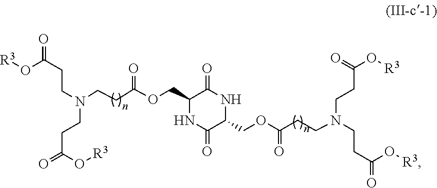
or a pharmaceutically acceptable salt thereof.

84. The cationic lipid of claim 79 or 80, wherein each R² is CH₃.

85. The cationic lipid of claim 84, having the following structure: 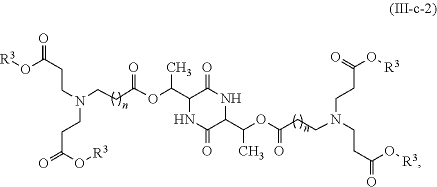
or a pharmaceutically acceptable salt thereof, optionally wherein the cationic lipid has the following structure: 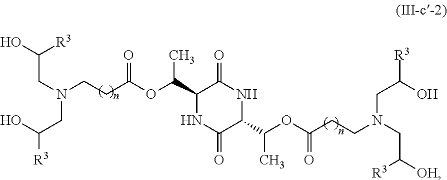
or a pharmaceutically acceptable salt thereof.

86. The cationic lipid of any one of claims 79-85, wherein (i) each n is 1; (ii) each n is 2; or (iii) each n is 3.

87. The cationic lipid of any one of claims 61-63, having the following structure: 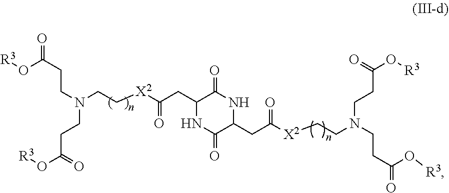
or a pharmaceutically acceptable salt thereof, wherein
each n is independently an integer having a value of 1 to 9; and
each X² is independently 0 or S.

88. The cationic lipid of claim 87, having the following structure: 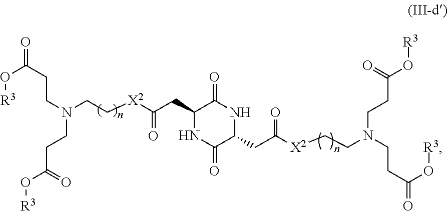
or a pharmaceutically acceptable salt thereof.

89. The cationic lipid of claim 87 or 88, wherein each n is 1.

90. The cationic lipid of claim 87 or 88, wherein each n is 2.

91. The cationic lipid of claim 87 or 88, wherein each n is 3.

92. The cationic lipid of any one of claims 87-91, wherein each X² is S.

93. The cationic lipid of claim 92, having the following structure, 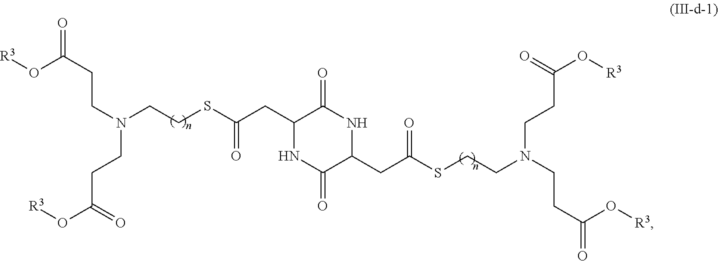
or a pharmaceutically acceptable salt thereof.

94. The cationic lipid of any one of claims 87-91, wherein each X² is O.

95. The cationic lipid of claim 94, having the following structure, 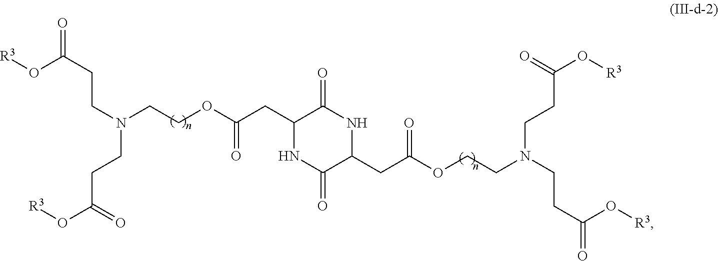
or a pharmaceutically acceptable salt thereof.

96. The cationic lipid of any one of claims 61-63, having the following structure: 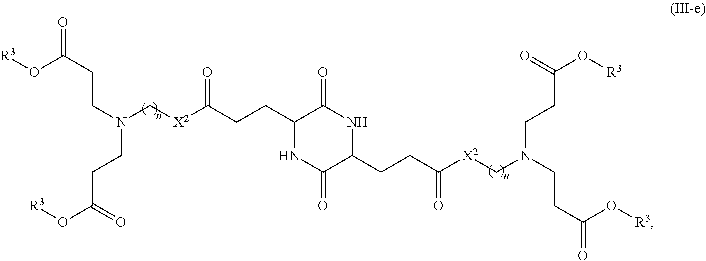
or a pharmaceutically acceptable salt thereof, wherein
each n is independently an integer having a value of 2 to 10; and
each X² is independently 0 or S.

97. The cationic lipid of claim 96, having the following structure: 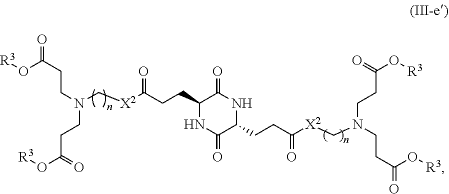
or a pharmaceutically acceptable salt thereof.

98. The cationic lipid of claim 96 or 97, wherein each n is 2.

99. The cationic lipid of claim 96 or 97, wherein each n is 3.

100. The cationic lipid of claim 96 or 97, wherein each n is 4.

101. The cationic lipid of any one of claims 96-100, wherein each X² is S.

102. The cationic lipid of claim 101, having the following structure, 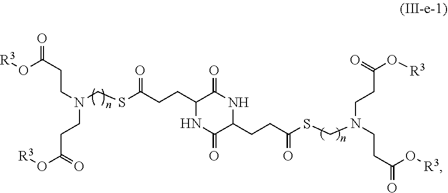
or a pharmaceutically acceptable salt thereof.

103. The cationic lipid of any one of claims 96-100, wherein each X² is O.

104. The cationic lipid of claim 103, having the following structure, 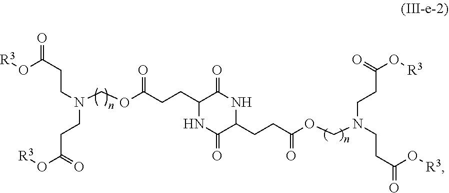
or a pharmaceutically acceptable salt thereof.

105. The cationic lipid of any one of claims 61-63, having the following structure: 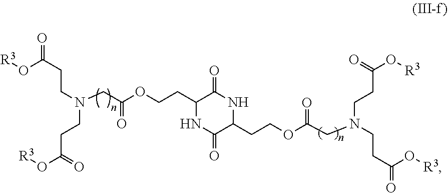
or a pharmaceutically acceptable salt thereof, wherein
each n is independently an integer of having a value of 2 to 10.

106. The cationic lipid of claim 105, having the following structure: 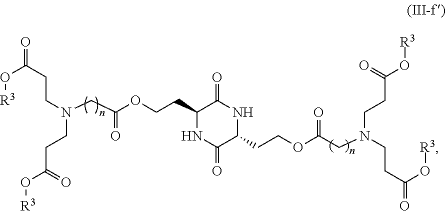
or a pharmaceutically acceptable salt thereof.

107. The cationic lipid of claim 105 or 106, wherein each n is 2.

108. The cationic lipid of claim 105 or 106, wherein each n is 3.

109. The cationic lipid of claim 105 or 106, wherein each n is 4.

110. The cationic lipid of any one of claims 61 to 109, wherein each R³ is independently selected from C₆-C₂₀ aliphatic.

111. The cationic lipid of any one of claims 61-63, having the following structure: 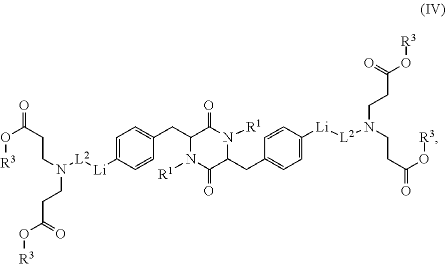
or a pharmaceutically acceptable salt thereof, wherein
each R¹ is independently H or C₁-C₆ aliphatic;
each L¹ is independently an ester, thioester, disulfide, or anhydride group;
each L² is independently C₂-C₁₀ aliphatic;
each R³ is independently C₆-C₃₀ aliphatic.

112. The cationic lipid of claim 111, wherein each R¹ is independently H or C₁-C₆ alkyl.

113. The cationic lipid of claim 111 or 112, wherein each R¹ is H.

114. The cationic lipid of any one of claims 111-113, having the following structure: 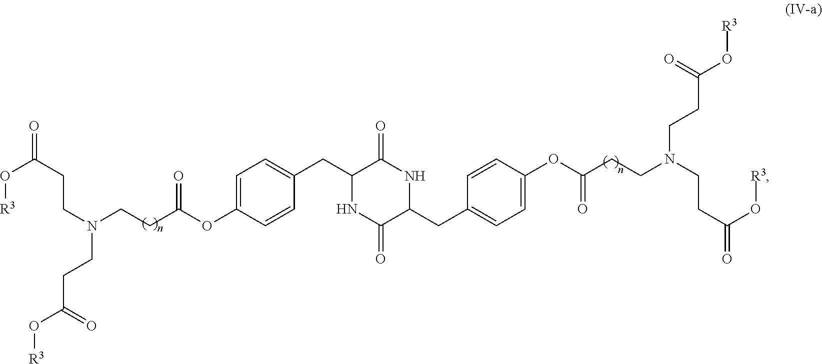
or a pharmaceutically acceptable salt thereof, wherein each n is an integer having a value of 1 to 9.

115. The cationic lipid of claim 114, having the following structure: 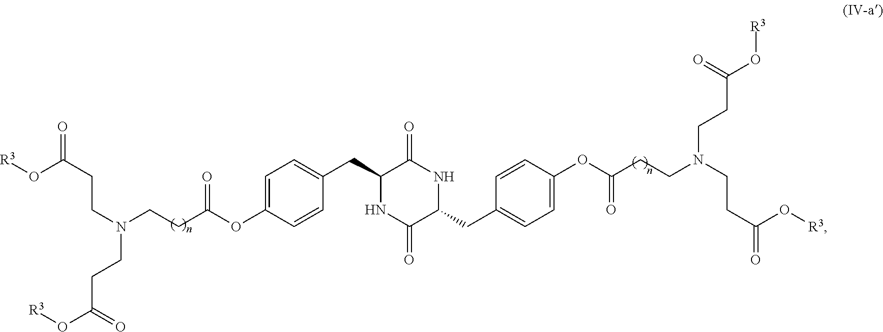
or a pharmaceutically acceptable salt thereof.

116. The cationic lipid of claim 114 or 115, wherein (i) each n is 1; (ii) each n is 2; or (iii) each n is 3.

117. The cationic lipid of any one of claims 111 to 116, wherein each R³ is independently selected from C₈ to C₂₀ aliphatic.

118. The cationic lipid of any one of claims 1-116, wherein each R³ is unsubstituted C₆-C₂₀ alkyl.

119. The cationic lipid of claim 117, wherein each R³ is C₆H₁₃, C₈H₁₇, C₁₀H₂₁, C₁₂H₂₅, C₁₄H₂₉, C₁₆H₃₃, or C₁₈H₃₇.

120. The cationic lipid of claim 118, wherein each R³ is C₁₀H₂₁.

121. The cationic lipid of any one of claims 1-116, wherein each R³ is substituted C₆-C₂₀ alkyl.

122. The cationic lipid of claim 121, wherein R³ comprises a substituent that is —O—C(O)R′ or —C(O)—OR′, wherein R′ is C₁-C₁₆ alkyl.

123. The cationic lipid of claim 122, wherein R³ is C₆-C₁₀ alkyl substituted by —O—C(O)C₇H₁₅ or —C(O)—O—(CH₂)₂CH(C₅H₁₁)₂.

124. The cationic lipid of claim 122, wherein each R³ is —(CH₂)₉—O—C(O)C₇H₁₅ or —(CH₂)₈C(O)—O—(CH₂)₂CH(C₅H₁₁)₂.

125. The cationic lipid of any one of claims 1-116, wherein each R³ is unsubstituted C₆-C₂₀ alkenyl.

126. The cationic lipid of claim 125, wherein each R³ is unsubstituted monoalkenyl, unsubstituted dienyl, or unsubstituted trienyl.

127. The cationic lipid of claim 125 or 126, wherein each R³ is —(CH₂)ₒR′, wherein o is 6, 7, 8, 9, or 10, and R′ is

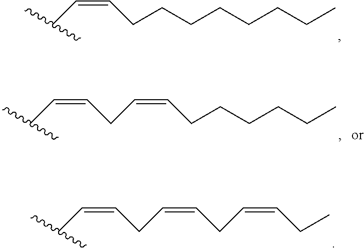 [SMILES: */C=C\CCCCCCCC ; */C=C\C/C=C\CCCCCC ; */C=C\C/C=C\C/C=C\CC]

128. The cationic lipid of claim 125, wherein each R³ is C₁₆H₃₁ or C₁₆H₂₉.

129. The cationic lipid of any one of claims 1-116, wherein each R³ is unsubstituted C₆-C₂₀ alkynyl.

130. A cationic lipid that is any one of Compounds 1-552, or a pharmaceutically acceptable salt thereof.

131. A composition comprising an mRNA encoding a protein, encapsulated within a liposome, wherein the liposome comprises a cationic lipid according to any one of claims 1-130.

132. The composition of claim 131, comprising an mRNA encoding for cystic fibrosis transmembrane conductance regulator (CFTR) protein.

133. The composition of claim 131, comprising an mRNA encoding for ornithine transcarbamylase (OTC) protein.

134. A composition comprising a nucleic acid encapsulated within a liposome, wherein the liposome comprises a cationic lipid according to any one of claims 1-130.

135. The composition of claim 134, wherein the nucleic acid is an mRNA encoding a peptide or protein.

136. The composition of claim 135, wherein the mRNA encodes a peptide or protein for use in the delivery to or treatment of the lung of a subject or a lung cell.

137. The composition of claim 136, wherein the mRNA encodes cystic fibrosis transmembrane conductance regulator (CFTR) protein.

138. The composition of claim 135, wherein the mRNA encodes a peptide or protein for use in the delivery to or treatment of the liver of a subject or a liver cell.

139. The composition of claim 138, wherein the mRNA encodes ornithine transcarbamylase (OTC) protein.

140. The composition of claim 131 or 134, wherein the mRNA encodes a peptide or protein for use in vaccine.

141. The composition of claim 140, wherein the mRNA encodes an antigen.

142. The composition of any one of claims 131-141, comprising one or more cationic lipids, one or more PEG-modified lipids, and/or one or more helper lipids.

143. The composition of claim 142, wherein the one or more helper lipids is 1,2-dierucoyl-sn-glycero-3-phosphoethanolamine (DEPE).

144. The composition of claim 143, wherein the one or more helper lipids is dioleoylphosphatidylethanolamine (DOPE).
